(19) United States
(12) Patent Application Publication
(10) Pub. No.: US 2021/0130473 A1
(43) Pub. Date: May 6, 2021

(54) TGFßR1 INHIBITOR-ASGR ANTIBODY CONJUGATES AND USES THEREOF

(71) Applicant: Silverback Therapeutics, Inc., Seattle, WA (US)

(72) Inventors: Peter R. BAUM, Seattle, WA (US); Robert DUBOSE, Seattle, WA (US); Valerie ODEGARD, Seattle, WA (US); Philip TAN, Seattle, WA (US); Peter A. THOMPSON, Seattle, WA (US); Sean W. SMITH, Seattle, WA (US); Brenda STEVENS, Seattle, WA (US)

(21) Appl. No.: 17/066,897

(22) Filed: Oct. 9, 2020

Related U.S. Application Data
(60) Provisional application No. 62/912,856, filed on Oct. 9, 2019.
(60) Provisional application No. 63/047,150, filed on Jul. 1, 2020.

(51) Int. Cl. C07K 16/28; A61K 31/444; A61K 31/4709; A61K 47/68

(52) U.S. Cl. CPC C07K 16/2851; A61K 31/444; A61K 31/4709; A61K 47/6859; C07K 2317/24; C07K 2317/565; C07K 2317/92; C07K 2317/622; C07K 2317/33; A61K 47/6803

(57) ABSTRACT

Various conjugates and compositions thereof are disclosed for use in the treatment of a liver disease, such as liver cancer and liver fibrosis. The compositions comprise conjugates, wherein the conjugates are comprised of an antibody or antibody construct specific for ASGR1 or ASGR2 attached to a TGFβR1 inhibitor via a linker. Additionally provided are the methods of preparation of the conjugates and compositions thereof.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 860234_407_SEQUENCE_LISTING.txt. The text file is 367 KB, was created on Oct. 6, 2020, and is being submitted electronically via EFS-Web.

### BACKGROUND

Fibrosis is the formation of excess fibrous connective tissue or scar tissue in an organ or tissue in a reparative or reactive process. Fibrosis can occur in many tissues within the body, typically as a result of inflammation or damage, which include the lungs, liver, heart, and brain. Scar tissue blocks arteries, immobilizes joints and damages internal organs, wreaking havoc on the body's ability to maintain vital functions. Every year, millions of people are hospitalized due to the damaging effects of fibrosis. However, current therapeutics for treating fibrotic diseases are lacking or have drawbacks. Thus, there remains a considerable need for alternative or improved treatments for fibrotic diseases.

### SUMMARY

In various aspects, a TGFβR1 inhibitor conjugate comprises:

a) an antibody construct comprising an antigen binding domain that specifically binds to a first antigen on a liver cell, wherein the first antigen is ASGR1 (such as human ASGR1);

b) a TGFβR1 inhibitor, e.g., a compound of Formula (A-I) or Formula (B-I); and

c) a linker covalently attached to the TGFβR1 inhibitor and to the antibody construct. In some embodiments, the antibody construct comprises an Fc domain; wherein the antibody construct or the antigen binding domain specific for ASGR1 comprises a heavy chain variable region (VH) and a light chain variable region (VL), wherein:
(i) the VH comprises a CDR1 (VH-CDR1) comprising the amino acid sequence of SEQ ID NO:1, a VH-CDR2 comprising the amino acid sequence selected from SEQ ID NO:8, a VH-CDR3 comprising the amino acid sequence of SEQ ID NO:13; and the VL comprises a CDR1 (VL-CDR1) comprising the amino acid sequence of SEQ ID NO:18, a VL-CDR2 comprising the amino acid sequence selected from any one of SEQ ID NO:23, and a VL-CDR3 comprising the amino acid sequence of SEQ ID NO:33;
(ii) the VH comprises a CDR1 (VH-CDR1) comprising the amino acid sequence of SEQ ID NO:1, a VH-CDR2 comprising the amino acid sequence selected from SEQ ID NO:6, a VH-CDR3 comprising the amino acid sequence of SEQ ID NO:13; and the VL comprises a CDR1 (VL-CDR1) comprising the amino acid sequence of SEQ ID NO:18, a VL-CDR2 comprising the amino acid sequence selected from any one of SEQ ID NO:23, and a VL-CDR3 comprising the amino acid sequence of SEQ ID NO:33;
(iii) the VH comprises a CDR1 (VH-CDR1) comprising the amino acid sequence of SEQ ID NO:2, a VH-CDR2 comprising the amino acid sequence selected from SEQ ID NO:9, a VH-CDR3 comprising the amino acid sequence of SEQ ID NO:14; and the VL comprises a CDR1 (VL-CDR1) comprising the amino acid sequence of SEQ ID NO:19, a VL-CDR2 comprising the amino acid sequence selected from any one of SEQ ID NO:26, and a VL-CDR3 comprising the amino acid sequence of SEQ ID NO:34;
(iv) the VH comprises a CDR1 (VH-CDR1) comprising the amino acid sequence of SEQ ID NO:1, a VH-CDR2 comprising the amino acid sequence selected from any one of SEQ ID NOS:6-8, a VH-CDR3 comprising the amino acid sequence of SEQ ID NO:13; and the VL comprises a CDR1 (VL-CDR1) comprising the amino acid sequence of SEQ ID NO:18, a VL-CDR2 comprising the amino acid sequence selected from any one of SEQ ID NOS:23-25, and a VL-CDR3 comprising the amino acid sequence of SEQ ID NO:33;
(v) the VH comprises a CDR1 (VH-CDR1) comprising the amino acid sequence of SEQ ID NO:2, a VH-CDR2 comprising the amino acid sequence of SEQ ID NO:9, a VH-CDR3 comprising the amino acid sequence of SEQ ID NO:14; and the VL comprises a CDR1 (VL-CDR1) comprising the amino acid sequence of SEQ ID NO:19, a VL-CDR2 comprising the amino acid sequence of SEQ ID NO:26 or SEQ ID NO:27, and a VL-CDR3 comprising the amino acid sequence of SEQ ID NO:34;
(vi) the VH comprises a CDR1 (VH-CDR1) comprising the amino acid sequence of SEQ ID NO:5, a VH-CDR2 comprising the amino acid sequence of SEQ ID NO:12, a VH-CDR3 comprising the amino acid sequence of SEQ ID NO:17; and the VL comprises a CDR1 (VL-CDR1) comprising the amino acid sequence of SEQ ID NO:22, a VL-CDR2 comprising the amino acid sequence of SEQ ID NO:32, and a VL-CDR3 comprising the amino acid sequence of SEQ ID NO:37;
(vii) the VH comprises a CDR1 (VH-CDR1) comprising the amino acid sequence of SEQ ID NO:3, a VH-CDR2 comprising the amino acid sequence of SEQ ID NO:10, a VH-CDR3 comprising the amino acid sequence of SEQ ID NO:15; and the VL comprises a CDR1 (VL-CDR1) comprising the amino acid sequence of SEQ ID NO:20, a VL-CDR2 comprising the amino acid sequence selected from any one of SEQ ID NOS:28-30, and a VL-CDR3 comprising the amino acid sequence of SEQ ID NO:35; or
(viii) the VH comprises a CDR1 (VH-CDR1) comprising the amino acid sequence of SEQ ID NO:4, a VH-CDR2 comprising the amino acid sequence of SEQ ID NO:11, a VH-CDR3 comprising the amino acid sequence of SEQ ID NO:16; and the VL comprises a CDR1 (VL-CDR1) comprising the amino acid sequence of SEQ ID NO:21, a VL-CDR2 comprising the amino acid sequence of SEQ ID NO:31, and a VL-((CDR3 comprising the amino acid sequence of SEQ ID NO:3.

In some aspects, the conjugate is represented by Formula (I):

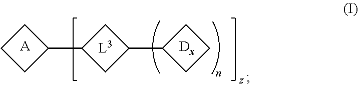

wherein: A is the antibody construct (e.g., anti-AGSR1 antibody or antigen binding domain thereof); L³ is the linker; Dₓ is the TGFβR1 inhibitor; n is selected from 1 to 20; and z is selected from 1 to 20.

In another aspect, the present disclosure relates to a pharmaceutical composition comprising a TGFβR1 inhibitor conjugate or a conjugate of Formula (I) as described herein and a pharmaceutically acceptable carrier.

In another aspect, the present disclosure relates to a method for treating a disease mediated by TGFβR1 activity in a subject in need thereof, such as liver cancer or liver fibrosis, comprising administering to the subject an effective amount of a TGFβR1 inhibitor or a conjugate thereof or a conjugate of Formula (I) or a pharmaceutical composition comprising a TGFβR1 inhibitor or a conjugate thereof or a conjugate of Formula (I) as described herein.

In another aspect, the present disclosure provides a method for enhancing an immune response (e.g., an anti-cancer immune response) in a subject in need thereof, comprising administering to the subject an effective amount of a TGFβR1 inhibitor or a conjugate thereof or a conjugate of Formula (I) or a pharmaceutical composition comprising a TGFβR1 inhibitor or a conjugate thereof or a conjugate of Formula (I) as described herein.

### BRIEF DESCRIPTION OF DRAWINGS

FIGS. 1A to 1D show the binding of various mouse and humanized anti-ASGR1 antibodies to human ASGR1-expressing cell line HepG2.

FIGS. 2A and 2B show binding of anti-human ASGR1 mAbs to (A) rat ASGR1 or (B) Cynomologous ASGR1, when transiently expressed by CHO-S cells.

### DETAILED DESCRIPTION

Additional aspects and advantages of the present disclosure will become apparent to those skilled in this art from the following detailed description, wherein illustrative aspects of the present disclosure are shown and described. As will be appreciated, the present disclosure is capable of other and different aspects, and its several details are capable of modifications in various respects, all without departing from the disclosure. Accordingly, the descriptions are to be regarded as illustrative in nature, and not as restrictive.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Activin receptor-like kinase 5 (ALK5), which is also commonly known as transforming growth factor beta receptor 1 (TGFβR1), is a serine/threonine kinase transmembrane receptor. It is a part of the TGFβ signaling pathway and is involved in signal transduction from the cell surface to the cytoplasm. The TGFβ signaling pathway regulates gene expression of genes involved in cellular processes such as differentiation, apoptosis, wound healing, and cell growth. In the absence of TGFβ ligands, ALK5 remains a homodimeric cell surface receptor. However, ligand binding to type II TGFβ receptor (TGFβR2) induces the formation of the TGFβR1/TGFβR2 complex, which leads to phosphorylation of Mothers Against Decapentaplegic homolog 2 (Smad2) and Mothers Against Decapentaplegic homolog 3 (Smad3) and subsequent modulation of a number of downstream signaling targets involved in the regulation of gene expression.

ALK5 inhibitors and conjugates thereof may be useful for treatment and/or prevention, e.g., vaccination, of cancer, autoimmune diseases, inflammation, fibrosis, sepsis, allergy, asthma, graft rejection, graft-versus-host disease, immunodeficiencies, and infectious diseases.

In some aspects, an ALK5 inhibitor has an IC₅₀ value of ranging from about 0.1 nM to about 1000 nM, from about 0.1 nm to about 100 nM, or from about 0.1 nM to about 80 nM in an ALK5 enzyme inhibition assay and/or in a TGFβR1 reporter assay. Exemplary ALK5 enzyme inhibition and TGFβR1 reporter assays are as set forth in the examples section.

### Definitions

Unless defined otherwise, all technical and scientific terms used herein have the same meaning as is commonly understood by one of skill in the art to which this disclosure belongs.

As used in the specification and claims, the singular form “a,” “an,” and “the” includes plural references unless the context clearly dictates otherwise.

The term “about” as used herein in the context of a number refers to a range centered on that number and spanning 10% less than that number and 10% more than that number. The term “about” used in the context of a range refers to an extended range spanning 10% less than that the lowest number listed in the range and 10% more than the greatest number listed in the range.

It should be understood that the terms “a” and “an” as used herein refer to “one or more” of the enumerated components. The use of the alternative (e.g., “or”) should be understood to mean either one, both, or any combination thereof of the alternatives. As used herein, the terms “include” and “comprise” are used synonymously.

The phrase “at least one of” when followed by a list of items or elements refers to an open ended set of one or more of the elements in the list, which may, but does not necessarily, include more than one of the elements.

As used herein, a “TGFβR1 inhibitor” refers to a compound that reduces, minimizes, or inactivates activin receptor-linked kinase 5 (ALK5) activity (e.g., directly inhibiting serine/threonine kinase activity or indirectly inhibiting downstream TGFβ-dependent signaling activity, such as SBE-mediated responsiveness to TGFβ and SMAD proteins) by about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% as compared to untreated ALK5. The terms ALK5 and TGF-βR1 can be used interchangeably.

As used herein, a “liver cell” refers to any cell type associated with normal liver tissue. For example, a liver cell can be a canalicular cell, a Kupffer cell, a hepatocyte, sinusoidal endothelial cell, or a stellate cell.

As used herein, an “immune cell” refers to a T cell, B cell, NK cell, NKT cell, or an antigen presenting cell. In some embodiments, an immune cell is a T cell, B cell, NK cell, or NKT cell. In some embodiments, an immune cell is an antigen presenting cell. In some embodiments, an immune cell is not an antigen presenting cell.

As used herein, “identical” or “identity” refer to the similarity between a DNA, RNA, nucleotide, amino acid, or protein sequence to another DNA, RNA, nucleotide, amino acid, or protein sequence. Identity can be expressed in terms of a percentage of sequence identity of a first sequence to a second sequence. Percent (%) sequence identity with respect to a reference DNA sequence can be the percentage of DNA nucleotides in a candidate sequence that are identical with the DNA nucleotides in the reference DNA sequence after aligning the sequences. Percent (%) sequence identity with respect to a reference amino acid sequence can be the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity.

As used herein, the term “antibody” refers to an immunoglobulin molecule that specifically binds to, or is immunologically reactive toward, a specific antigen. The portion of the antibody that binds a specific antigen may be referred to as an “antigen binding domain.” The term antibody can include, for example, polyclonal, monoclonal, genetically engineered, and antigen binding fragments thereof. An antibody can be, for example, murine, chimeric, humanized, a heteroconjugate, bispecific, diabody, triabody, or tetrabody. An antigen binding fragment can include, for example, a Fab′, F(ab′)₂, Fab, Fv, rIgG, scFv, hcAbs (heavy chain antibodies), a single domain antibody, V_{HH}, V_{NAR}, sdAbs, or nanobody.

As used herein, “recognize” refers to the specific association or specific binding between an antigen binding domain and an antigen. Specific association or specific binding does not require that the antigen binding domain does not associate with or bind to any other antigen, but rather that it preferentially associates with or binds to the antigen, as compared to association with or binding to an unrelated antigen.

As used herein, a “liver cell antigen” refers to an antigenic substance expressed on a liver cell that can be recognized by an antibody or binding domain, and is preferentially expressed on a non-cancerous liver cell as compared to cells from other tissues.

As used herein, a “viral antigen” refers to an antigenic substance associated a virus, a viral infection, or combination thereof. A “viral antigen from a virus infecting a liver cell” refers to antigenic substance associated with a virus that is infecting or has infected a liver cell and that can trigger an immune response in a host.

As used herein, an “antibody construct” refers to a construct that contains an antigen binding domain and an Fc domain.

As used herein, an “Fc domain” or “Fc region” refers to a Fc portion of an antibody that can bind to a Fc receptor, such as a Fcgamma receptor or a FcRn.

As used herein, an “antigen binding domain” refers to an antigen binding domain of a conjugate or construct that specifically binds an antigen.

As used herein, a “conjugate” refers to an antibody construct attached to at least one TGFβR1 inhibitor, optionally via a linker.

As used herein, a “bispecific antibody construct” refers to an antibody construct comprising a first antigen binding domain and a second antigen binding domain.

As used herein, a “bispecific antibody conjugate” refers to an antibody construct comprising a first antigen binding domain and a second antigen binding domain and that is attached to at least one TGFβR1 inhibitor via a linker.

As used herein, the abbreviations for the natural L-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid. In some aspects, X can be asparagine (N), glutamine (Q), histidine (H), lysine (K), or arginine (R).

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The term “C_{x-y}” when used in conjunction with a chemical moiety, such as alkyl, alkenyl, or alkynyl is meant to include groups that contain from x to y carbons in the chain. For example, the term “C₁₋₆ alkyl” refers to substituted or unsubstituted saturated hydrocarbon groups, including straight-chain alkyl and branched-chain alkyl groups that contain from 1 to 6 carbons. The term —C_{x-y} alkylene- refers to a substituted or unsubstituted alkylene chain with from x to y carbons in the alkylene chain. For example —C₁₋₆ alkylene- may be selected from methylene, ethylene, propylene, butylene, pentylene, and hexylene, any one of which is optionally substituted.

The terms “C_{x-y} alkenyl” and “C_{x-y} alkynyl” refer to substituted or unsubstituted unsaturated aliphatic groups analogous in length and possible substitution to the alkyls described above, but that contain at least one double or triple bond, respectively. The term —C_{x-y} alkenylene- refers to a substituted or unsubstituted alkenylene chain with from x to y carbons in the alkenylene chain. For example, —C₂₋₆ alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y} alkynylene-refers to a substituted or unsubstituted alkynylene chain with from x to y carbons in the alkenylene chain. For example, —C₂₋₆ alkenylene- may be selected from ethynylene, propynylene, butynylene, pentynylene, and hexynylene, any one of which is optionally substituted. An alkynylene chain may have one triple bond or more than one triple bond in the alkynylene chain.

“Alkyl” refers to a monovalent hydrocarbon consisting solely of carbon and hydrogen, containing no unsaturation, and preferably having from one to twelve carbon atoms, for example, methyl, ethyl, propyl, isopropyl, butyl, and the like. In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl or methyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). Unless stated otherwise specifically in the specification, an alkyl chain is optionally substituted by one or more substituents such as those substituents described herein.

“Alkylene” refers to a divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). Unless stated otherwise specifically in the specification, an alkylene chain is optionally substituted by one or more substituents such as those substituents described herein.

“Alkenyl” refers to a monovalent hydrocarbon chain consisting solely of carbon and hydrogen, containing at least one carbon-carbon double bond, and preferably having from two to twelve carbon atoms. The alkenyl chain is attached to the rest of the molecule through a single bond. In other embodiments, an alkenyl comprises two to five carbon atoms (i.e., C₂-C₅ alkenyl). In other embodiments, an alkenyl comprises two to four carbon atoms (i.e., C₂-C₄ alkenyl). In other embodiments, an alkenyl comprises two to three carbon atoms (i.e., C₂-C₃ alkenyl). In other embodiments, an alkenyl comprises two carbon atom (i.e., C₂ alkenyl). In other embodiments, an alkenyl comprises five to eight carbon atoms (i.e., C₅-C₈ alkenyl). In other embodiments, an alkenyl comprises three to five carbon atoms (i.e., C₃-C₅ alkenyl). Unless stated otherwise specifically in the specification, an alkenyl chain is optionally substituted by one or more substituents such as those substituents described herein.

“Alkenylene” refers to a divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene). Unless stated otherwise specifically in the specification, an alkenylene chain is optionally substituted by one or more substituents such as those substituents described herein.

“Alkynyl” refers to a monovalent hydrocarbon chain consisting solely of carbon and hydrogen, containing at least one carbon-carbon triple bond, and preferably having from two to twelve carbon atoms. The alkynyl chain is attached to the rest of the molecule through a single bond. In other embodiments, an alkynyl comprises two to five carbon atoms (i.e., C₂-C₅ alkynyl). In other embodiments, an alkynyl comprises two to four carbon atoms (i.e., C₂-C₄ alkynyl). In other embodiments, an alkynyl comprises two to three carbon atoms (i.e., C₂-C₃ alkynyl). In other embodiments, an alkynyl comprises two carbon atom (i.e., C₂ alkynyl). In other embodiments, an alkynyl comprises five to eight carbon atoms (i.e., C₅-C₈ alkynyl). In other embodiments, an alkynyl comprises three to five carbon atoms (i.e., C₃-C₅ alkynyl). Unless stated otherwise specifically in the specification, an alkynyl chain is optionally substituted by one or more substituents such as those substituents described herein.

“Alkynylene” refers to a divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene). Unless stated otherwise specifically in the specification, an alkynylene chain is optionally substituted by one or more substituents such as those substituents described herein.

“Heteroalkyl” refers to a monovalent hydrocarbon chain including at least one heteroatom in the chain, containing no unsaturation, and preferably having from one to twelve carbon atoms and from one to 6 heteroatoms, e.g., —O—, —NH—, —S—. The heteroalkyl is attached to the rest of the molecule through a single bond. In other embodiments, a heteroalkyl comprises one to five carbon atoms and from one to three heteroatoms. In other embodiments, a heteroalkyl comprises one to four carbon atoms and from one to three heteroatoms. In other embodiments, a heteroalkyl comprises one to three carbon atoms and from one to two heteroatoms. In other embodiments, a heteroalkyl comprises one to two carbon atoms and from one to two heteroatoms. In other embodiments, a heteroalkyl comprises one carbon atom and from one to two heteroatoms. In other embodiments, a heteroalkyl comprises five to eight carbon atoms and from one to four heteroatoms. In other embodiments, a heteroalkyl comprises two to five carbon atoms and from one to three heteroatoms. In other embodiments, a heteroalkyl comprises three to five carbon atoms and from one to three heteroatoms. Unless stated otherwise specifically in the specification, a heteroalkyl chain is optionally substituted by one or more substituents such as those substituents described herein.

“Heteroalkylene” refers to a divalent hydrocarbon chain including at least one heteroatom in the chain, containing no unsaturation, and preferably having from one to twelve carbon atoms and from one to 6 heteroatoms, e.g., —O—, —NH—, —S—. The heteroalkylene chain is attached to the rest of the molecule through a single bond and to the radical group through a single bond. The points of attachment of the heteroalkylene chain to the rest of the molecule and to the radical group are through the terminal atoms of the chain. In other embodiments, a heteroalkylene comprises one to five carbon atoms and from one to three heteroatoms. In other embodiments, a heteroalkylene comprises one to four carbon atoms and from one to three heteroatoms. In other embodiments, a heteroalkylene comprises one to three carbon atoms and from one to two heteroatoms. In other embodiments, a heteroalkylene comprises one to two carbon atoms and from one to two heteroatoms. In other embodiments, a heteroalkylene comprises one carbon atom and from one to two heteroatoms. In other embodiments, a heteroalkylene comprises five to eight carbon atoms and from one to four heteroatoms. In other embodiments, a heteroalkylene comprises two to five carbon atoms and from one to three heteroatoms. In other embodiments, a heteroalkylene comprises three to five carbon atoms and from one to three heteroatoms. Unless stated otherwise specifically in the specification, a heteroalkylene chain is optionally substituted by one or more substituents such as those substituents described herein.

The term “carbocycle” or “carbocyclyl” as used herein refers to a saturated, unsaturated or aromatic ring in which each atom of the ring is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, and naphthyl. The term “unsaturated carbocycle” refers to carbocycles with at least one degree of unsaturation and excluding aromatic carbocycles. Examples of unsaturated carbocycles include cyclohexadiene, cyclohexene, and cyclopentene.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

The term “cycloalkyl” refers to a saturated ring in which each atom of the ring is carbon. Cycloalkyl may include monocyclic and polycyclic rings such as 3- to 10-membered monocyclic rings, 6- to 12-membered bicyclic rings, and 6- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), decalinyl, 7,7 dimethyl bicyclo[2.2.1]heptanyl, and the like.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like.

The term “heterocycle” or “heterocyclyl” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. The term “unsaturated heterocycle” refers to heterocycles with at least one degree of unsaturation and excluding aromatic heterocycles. Examples of unsaturated heterocycles include dihydropyrrole, dihydrofuran, oxazoline, pyrazoline, and dihydropyridine.

The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also includes polycyclic ring systems having two or more rings in which two or more carbons are common to two adjoining rings wherein at least one of the rings is heteroaromatic, e.g., the other rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

The term “heterocycloalkyl” refers to a saturated ring with carbon atoms and at least one heteroatom. Exemplary heteroatoms include N, O, Si, P, B, and S atoms. Heterocycloalkyl may include monocyclic and polycyclic rings such as 3- to 10-membered monocyclic rings, 6- to 12-membered bicyclic rings, and 6- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

The term “substituted” refers to moieties having substituents replacing a hydrogen on one or more carbons or substitutable heteroatoms, e.g., —NH—, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂),

—R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)OR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro
(—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

“Protecting group” refers to a moiety, except alkyl groups, that when attached to a reactive group in a molecule masks, reduces or prevents that reactivity. Examples of protecting groups can be found in T. W. Greene and P. G. M. Wuts, Protective Groups in Organic Synthesis, 3.sup.rd edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative amino or amine protecting groups include, formyl, acyl groups (such as acetyl, trifluoroacetyl, and benzoyl), benzyl, alkoxycarbonyl (such as benzyloxycarbonyl (CBZ), and tert-butoxycarbonyl (Boc)), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), sulfonyl, and the like. Compounds described herein can include protecting groups (e.g., a hydrogen on a reactive nitrogen atom of a compound described herein can be replaced by an amino protecting group).

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

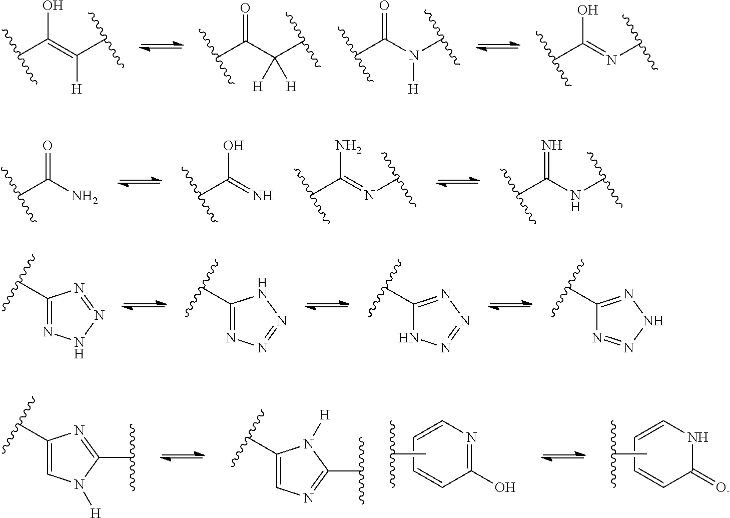

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley and Sons, Inc., 1981, herein incorporated by reference for this disclosure). Stereoisomers may also be obtained by stereoselective synthesis.

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds.

For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

### ASGR (Asialoglycoprotein Receptor) Antibody and Antibody Construct

A conjugate can comprise an antibody construct comprising an asialoglycoprotein receptor 1 (ASGR1) or an asialoglycoprotein receptor 2 (ASGR2) binding domain and an Fc domain. A conjugate as described herein comprises an antibody construct. An antibody construct comprises one or more ASGR1 or ASGR2 binding domains and an Fc domain. In some embodiments, an antibody construct comprises an antigen binding domain that specifically binds to ASGR1 and an Fc domain. In some embodiments, an antibody construct comprises an antigen binding domain that specifically binds to ASGR2 and an Fc domain.

An antibody construct can comprise a first antigen binding domain that specifically binds to ASGR1 or ASGR2, a second antigen binding domain that specifically binds to a second antigen, and an Fc domain. In some such embodiments, the antibody construct is a bispecific antibody construct.

A conjugate can comprise an antibody specific for an asialoglycoprotein receptor 1 (ASGR1) or an asialoglycoprotein receptor 2 (ASGR2), such as human ASGR1 or human ASGR2. A conjugate as described herein comprises an antibody. In some embodiments, an antibody construct comprises an antigen binding domain that specifically binds to ASGR1 and a wild-type or a mutated Fc domain. In some embodiments, an antibody construct comprises an antigen binding domain that specifically binds to ASGR2 and a wild-type or a mutated Fc domain.

An antibody or antibody construct of a conjugate of the instant disclosure comprises an antigen binding domain that binds ASGR1 or ASGR2. In a conjugate, an ASGR1 or ASGR2 antibody or antibody construct can be linked to a TGFβR1 inhibitor in such a way that the antibody or antibody construct can still bind to ASGR1 or ASGR2, respectively, and the Fc domain of the antibody or antibody construct can still bind to an FcR, resulting in FcR-mediated signaling. In a conjugate, an ASGR1 or ASGR2 antibody or antibody construct can be linked to a TGFβR1 inhibitor in such a way that the linking does not interfere with ability of the ASGR1 or ASGR2 binding domain of the antibody or antibody construct to bind to ASGR1 or ASGR2, respectively, the ability of the Fc domain of the antibody or antibody construct to bind to an FcR, or with FcR-mediated signaling resulting from the Fc domain of the antibody or antibody construct binding to an FcR. In a conjugate, a TGFβR1 inhibitor can be linked to an antibody or antibody construct in such a way the linking does not interfere with the ability of the TGFβR1 inhibitor to bind to its receptor. A conjugate can produce stronger immune stimulation and a greater therapeutic window than components of the conjugate alone.

Affinity can be the strength of the sum total of noncovalent interactions between a single binding site of a molecule, for example, an antibody, and the binding partner of the molecule, for example, an antigen. The affinity can also measure the strength of an interaction between an Fc domain of an antibody or antibody construct and the Fc receptor. Unless indicated otherwise, as used herein, “binding affinity” can refer to intrinsic binding affinity which reflects a 1:1 interaction between members of a binding pair (e.g., antibody and antigen or Fc domain and Fc receptor). The affinity of a molecule X for its partner Y can generally be represented by the dissociation constant (K_{d}). Affinity can be measured by common methods known in the art, including those described herein. K_{d} can be measured by any suitable assay. For example, K_{d} can be measured by a radiolabeled antigen binding assay (RIA). For example, K_{d} can be measured using surface plasmon resonance assays (e.g., using a BIACORE®-2000 or a BIACORE®-3000). Specific illustrative and exemplary embodiments for measuring binding affinity are described in the following.

In some embodiments, an ASGR1 or ASGR2 antibody or antibody construct provided herein can has a dissociation constant (K_{d}) of about 1 μM, about 100 nM, about 10 nM, about 5 nM, about 2 nM, about 1 nM, about 0.5 nM, about 0.1 nM, about 0.05 nM, about 0.01 nM, or about 0.001 nM or less (e.g., 10⁻⁸ M or less, e.g., from 10⁻⁸ M to 10⁻¹³ M, e.g., from 10⁻⁹ M to 10⁻¹³ M) for its target antigen. An affinity matured antigen-binding domain may have one or more alterations in one or more complementarity determining regions (CDRs), compared to a parent antigen binding domain, such alterations resulting in an improvement in the affinity of the antigen binding domain for antigen. Antibodies comprising such affinity-matured antigen-binding domains may bind to their antigen with a K_{d} of about 5×10⁻⁹ M, about 2×10⁻⁹ M, about 1×10⁻⁹ M, about 5×10⁻¹ M, about 2×10⁻⁹ M, about 1×10⁻¹⁰ M, about 5×10⁻¹¹ M, about 1×10⁻¹¹ M, about 5×10⁻¹² M, about 1×10⁻¹² M, or less. In some embodiments, the conjugate can have an increased affinity of at least 1.5-fold, 2-fold, 2.5-fold, 3-fold, 4-fold, 5-fold, 10-fold, 20-fold, or greater as compared to a conjugate without alterations in one or more complementarity determining regions of an antigen-binding domain.

The molar ratio of a conjugate refers to the average number of TGFβR1 inhibitors conjugated to the ASGR1 or ASGR2 antibody or antibody construct in a preparation of a conjugate. The molar ratio can be determined, for example, by Liquid Chromatography/Mass Spectrometry (LC/MS), in which the number of TGFβR1 inhibitors conjugated to the antibody or antibody construct can be directly determined. Additionally, as non-limiting examples, the molar ratio can be determined based on hydrophobic interaction chromatography (HIC) peak area, by liquid chromatography coupled to electrospray ionization mass spectrometry (LC-ESI-MS), by UV/Vis spectroscopy, by reversed-phase-HPLC (RP-HPLC), or by matrix-assisted laser desorption/ionization time of flight mass spectrometry (MALDI-TOF-MS).

In some embodiments, the molar ratio of TGFβR1 inhibitors to ASGR1 or ASGR2 antibody or antibody construct (also referred to as a Drug-to-Antibody Ratio or DAR) can be less than 8. In other embodiments, the DAR can be about 8, about 7, about 6.5, about 6, about 5.5, about 5, about 4.5, about 4, about 3.5, about 3, about 2.5, about 2, or 1. In certain embodiments, the DAR can range from 1 to about 8; from 2 to about 7, from about 3 to about 6, from about 4 to about 5

ASGR Binding Domain

An ASGR1 or ASGR2 binding domain can be an antigen-binding portion of an antibody or an antibody fragment that retains the ability to specifically bind to ASGR1 or ASGR2, respectively. An ASGR1 or ASGR2 binding domain typically recognizes a single antigen. An antibody construct typically comprises, for example, one or two ASGR1 or ASGR2 binding domains although more can be included in an antibody construct. An antibody construct can comprise two ASGR1 or ASGR2 binding domains in which each ASGR1 or ASGR2 binding domain recognizes the same antigen—ASGR1 or ASGR2, respectively. An antibody construct can comprise two ASGR1 or ASGR2 binding domains in which each ASGR1 or ASGR2 binding domain recognizes the same epitope on the antigen—ASGR1 or ASGR2, respectively. An antibody construct can comprise two ASGR1 or ASGR2 binding domains in which each ASGR1 or ASGR2 binding domain recognizes a different epitope on the same antigen—ASGR1 or ASGR2, respectively. In such embodiments, the antibody construct can comprise a first ASGR1 or ASGR2 binding domain and a second ASGR1 or ASGR2 binding domain. An antibody construct can comprise two ASGR1 or ASGR2 binding domains in which each ASGR1 or ASGR2 binding domain recognizes a different antigen—an antigen other than ASGR1 or ASGR2, respectively. An antibody construct can comprise three antigen binding domains in which each antigen binding domain recognizes a different antigen and wherein at least one of the three antigen binding domains is specific for ASGR1 or ASGR2. An antibody construct can comprise three antigen binding domains in which two of the antigen binding domains recognize the same antigen, wherein at least one of the three antigen binding domains is specific for ASGR1 or ASGR2. An ASGR1 or ASGR2 binding domain can be in a scaffold, in which a scaffold is a supporting framework for the ASGR1 or ASGR2 binding domain. An ASGR1 or ASGR2 binding domain can be in a non-antibody scaffold. An ASGR1 or ASGR2 binding domain can be in an antibody scaffold or antibody-like scaffold. An antibody construct can comprise an ASGR1 or ASGR2 binding domain in a scaffold. In any of the aforementioned binding domains specific for ASGR1 or ASGR2, the binding domains are specific for human ASGR1 or human ASGR2.

In some embodiments, an ASGR1 or ASGR2 binding domain comprises a heavy chain variable domain (VH) and a light chain variable domain (VL), a Fab′, F(ab′)₂, Fab, Fv, rIgG, scFv, hcAbs (heavy chain antibodies), a single domain antibody, V_{HH}, V_{NAR}, sdAbs, or nanobody. In some embodiments, an ASGR1 or ASGR2 binding domain is a domain of a non-antibody molecule that specifically binds to an ASGR1 or ASGR2, respectively, including, but not limited to, from a non-antibody scaffold, such as a DARPin, an affimer, an avimer, a knottin, a monobody, lipocalin, an anticalin, ‘T-body’, an affibody, a peptibody, an affinity clamp, an ectodomain, a receptor ectodomain, a receptor, a ligand, or a centryin.

An ASGR1 or ASGR2 binding domain of an antibody construct, for example an ASGR1 or ASGR2 binding domain from a monoclonal antibody, can comprise a light chain and a heavy chain. In one aspect, a monoclonal antibody specifically binds to an ASGR1 or ASGR2 present on the surface of a liver cell.

In certain embodiments, a conjugate of this disclosure can specifically bind to an ASGR1 or ASGR2 on a liver cell. In other embodiments, a conjugate can be capable of specifically binding to two or more antigens, wherein the antigens are the same or different, and at least one antigen is ASGR1 or ASGR2. The second antigen binding domain (not the ASGR1 or ASGR2 binding domain) can specifically bind to an antigen on a liver cell, wherein the antigen is a liver cell antigen. Each antigen can be expressed by a liver cell. A liver cell antigen can be a molecular marker, which is expressed on a liver cell when the liver cell is a cancer cell, and is preferentially expressed on a liver cell as compared to cells from other normal tissues. For example, a liver cell antigen can be expressed on a canalicular cell, Kupffer cell, hepatocyte, a stellate cell, or other liver cell type, or a combination thereof. The liver cell antigen can be a liver cell surface receptor. The liver cell antigen can be a hepatocyte antigen. The liver cell antigen can be expressed on a non-cancerous liver cell.

A liver cell antigen can include, but is not limited to, asialoglycoprotein receptor 1 (ASGR1), asialoglycoprotein receptor 2 (ASGR2), transferrin receptor 2 (TRF2), UDP glucuronosyltransferase 1 family, polypeptide A1 (UGT1A1), solute carrier family 22 member 7 (SLC22A7), solute carrier family 13 member 5 (SLC13A5), solute carrier family 22 member 1(SLC22A1), and complement component 9 (C9). In some embodiments, a liver cell antigen is selected from one or more of ASGR1, ASGR2, TRF2, UGT1A1, SLC22A7, SLC13A5, SLC22A1, and C9. In some embodiments, the liver cell antigen is selected from one or more of ASGR1, ASGR2, TRF2, UGT1A1, SLC22A7, SLC13A5, and SLC22A1. In some embodiments, the liver cell antigen is selected from one or more of ASGR1, ASGR2 and TRF2. The antigen binding domain can specifically bind to a liver cell antigen, wherein the liver cell antigen has an amino acid sequence that comprises at least 80% identity to an amino acid sequence of an antigen selected from one or more of human ASGR1, human ASGR2, and human TRF2. The antigen binding domain can specifically bind to a liver cell antigen, wherein the liver cell antigen has an amino acid sequence that comprises at least 85%, 90%, 95%, 98%, or 100% identity to an amino acid sequence of an antigen selected from human ASGR1, human ASGR2, and human TRF2. In some embodiments, the antigen binding domain can specifically bind to a liver cell antigen, wherein the liver cell antigen has an amino acid sequence that comprises at least 80% identity to an amino acid sequence of an antigen selected from ASGR1, ASGR2, TRF2, UGT1A1, SLC22A7, SLC13A5, SLC22A1, and C9. The antigen binding domain can specifically bind to a liver cell antigen, wherein the liver cell antigen has an amino acid sequence that comprises at least 85%, 90%, 95%, 98%, or 100% identity to an amino acid sequence of an antigen selected from ASGR1, ASGR2, TRF2, UGT1A1, SLC22A7, SLC13A5, SLC22A1, and C9. The antigen binding domain can specifically bind to a liver cell antigen, wherein the liver cell antigen has an amino acid sequence that comprises at least 80% identity to an amino acid sequence of an antigen selected from ASGR1, ASGR2, TRF2, UGT1A1, SLC22A7, SLC13A5, and SLC22A1. The antigen binding domain can specifically bind to a liver cell antigen, wherein the liver cell antigen has an amino acid sequence that comprises at least 85%, 90%, 95%, 98%, or 100% identity to an amino acid sequence of an antigen selected from ASGR1, ASGR2, TRF2, UGT1A1, SLC22A7, SLC13A5, and SLC22A1.

The specificity of an antigen binding domain to the antigen of a conjugate disclosed herein can be influenced by the presence of a TGFβR1 inhibitor. The antigen binding domain of a conjugate of the instant disclosure can bind to its antigen with at least about 30%, about 40%, about 50%, about 60%, about 70%, about 80%, about 85%, about 90%, about 95%, or about 100% of a specificity of the antigen binding domain to the antigen in the absence of the TGFβR1 inhibitor.

The affinity of an antigen binding domain to the antigen, such as ASGR1 or ASGR2, of a conjugate disclosed herein can be influenced by the presence of a TGFβR1 inhibitor. An antigen binding domain of the conjugate can bind to the antigen, such as ASGR1 or ASGR2, with at least about 30%, about 40%, about 50%, about 60%, about 70%, about 80%, about 85%, about 90%, about 95%, or about 100% of an affinity of the antigen binding domain to the antigen in the TGFβR1 inhibitor.

The K_{d} for binding of a first ASGR1 or ASGR2 binding domain of a conjugate to a first ASGR1 or ASGR2, respectively, in the presence of a TGFβR1 inhibitor can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the first ASGR1 or ASGR2 binding domain to the first antigen ASGR1 or ASGR2, respectively, of a conjugate in the absence of the TGFβR1 inhibitor conjugate. The K_{d} for binding of a first antigen ASGR1 or ASGR2 binding domain of a conjugate to the first antigen ASGR1 or ASGR2, respectively, in the presence of the TGFβR1 inhibitor conjugate can be less than 10 nM. The K_{d} for binding of a first antigen ASGR1 or ASGR2 binding domain of a conjugate to the first antigen ASGR1 or ASGR2, respectively, in the presence of the TGFβR1 inhibitor conjugate can be less than 100 nM, less than 50 nM, less than 20 nM, less than 5 nM, less than 1 nM, or less than 0.1 nM.

The K_{d} for binding of a second antigen binding domain of a conjugate to the second antigen in the presence of a TGFβR1 inhibitor conjugate can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the Kd for binding of the second antigen binding domain to the second antigen of a conjugate in the absence of the TGFβR1 inhibitor conjugate. The K_{d} for binding of a second antigen binding domain of a conjugate to the second antigen in the presence of the TGFβR1 inhibitor conjugate can be less than 10 nM. The K_{d} for binding of a second antigen binding domain of a conjugate to the second antigen in the presence of the TGFβR1 inhibitor conjugate can be less than 100 nM, less than 50 nM, less than 20 nM, less than 5 nM, less than 1 nM, or less than 0.1 nM. In contrast, the K_{d} for binding of a second antigen binding domain of conjugate to a second antigen in the presence of the TGFβR1 inhibitor conjugate when the first antigen binding domain is bound to the first antigen binding domain's antigen can be greater than 100 nM. The K_{d} for binding of a second antigen binding domain of a conjugate to a second antigen in the presence of the TGFβR1 inhibitor conjugate when the first binding domain is bound to the first antigen binding domain's antigen can be greater than 100 nM, greater than 200 nM, greater than 300 nM, greater than 400 nM, greater than 500 nM, or greater than 1000 nM.

Asialoglycoprotein receptor 1 (ASGR1) can have the amino acid sequence set forth in accession NP_001184145.1 or NP_001662.1. Asialoglycoprotein receptor 1 (ASGR1) can have an amino acid sequence that has at least about 90%, about 91%, about 92%, about 93%, about 94%, about 95%, about 96%, about 97%, about 98%, or about 99% identity to the amino acid sequence set forth in accession NP_001184145.1 or NP_001662.1. Asialoglycoprotein receptor 2 (ASGR2) can have the amino acid sequence set forth in accession NP_001172.1, NP_001188281.1, NP_550434.1, NP_550435.1, or NP_550436.1. Asialoglycoprotein receptor 2 (ASGR2) can have an amino acid sequence that has at least about 90%, about 91%, about 92%, about 93%, about 94%, about 95%, about 96%, about 97%, about 98%, or about 99% identity to the amino acid sequence set forth in accession NP_001172.1, NP_001188281.1, NP_550434.1, NP_550435.1, or NP_550436.1. Transferrin receptor 2 (TRF2) can have the amino acid sequence set forth in accession NP_001193784.1 or NP_003218.1. Transferrin receptor 2 (TRF2) can have an amino acid sequence that has at least about 90% identical to the amino acid sequence set forth in accession NP_001193784.1 or NP_003218.1. UDP glucuronosyltransferase 1 family, polypeptide A1 (UGT1A1) can have the amino acid sequence set forth in accession NP_000454.1. UDP glucuronosyltransferase 1 family, polypeptide A1 (UGT1A1) can have an amino acid sequence that has at least about 90% identical to the amino acid sequence set forth in accession NP_000454.1. Solute carrier family 22 member 7 (SLC22A7) can have the amino acid sequence set forth in accession NP_006663.2 or NP_696961.2. Solute carrier family 22 member 7 (SLC22A7) can have an amino acid sequence that has at least about 90% identical to the amino acid sequence set forth in accession NP_006663.2 or NP_696961.2. Solute carrier family 13 member 5 (SLC13A5) can have the amino acid sequence set forth in accession NP_001137310.1, NP_001271438.1, NP_001271439.1 or NP_808218.1. Solute carrier family 13 member 5 (SLC13A5) can have an amino acid sequence that has at least about 90% identical to the amino acid sequence set forth in accession NP_001137310.1, NP_001271438.1, NP_001271439.1 or NP_808218.1. Solute carrier family 22 member 1(SLC22A1) can have the amino acid sequence set forth in accession NP_003048.1 or NP_694857.1. Solute carrier family 22 member 1(SLC22A1) can have an amino acid sequence that has at least about 90% identical to the amino acid sequence set forth in accession NP_003048.1 or NP_694857.1. Complement component 9 (C9) can have the amino acid sequence set forth in accession NP_001728.1. Complement component 9 (C9) can have an amino acid sequence that has at least about 90% identical to the amino acid sequence set forth in accession NP_001728.1.

In some embodiments, an antibody construct is an antibody. In some embodiments, an antibody comprises an antigen binding domain and an Fc domain. In some embodiments, an antibody comprises two light chain polypeptides (light chains) and two heavy chain polypeptides (heavy chains), held together covalently by disulfide linkages. The heavy chain typically comprises a heavy chain variable region (VH) and a heavy chain constant region. The heavy chain constant region comprises three domains, CH1, CH2, and CH3. An Fc domain typically comprises heavy chain CH2 and CH3 domains. The light chain typically comprises a light chain variable region (VL) and a light chain constant region. The antigen-recognition regions of the antibody variable domains typically comprise six complementarity determining regions (CDRs), or hypervariable regions, that lie within the framework of the heavy chain variable region and light chain variable region at the N-terminal ends of the two heavy and two light chains. The constant domains provide the general framework of the antibody and may not be involved directly in binding the antibody to an antigen, but can be involved in various effector functions, such as participation of the antibody in antibody-dependent cellular cytotoxicity (ADCC).

An antibody can be any class, e.g., IgA, IgD, IgE, IgG, and IgM. Certain classes can be further divided into isotypes, e.g., IgG1, IgG2, IgG3, IgG4, IgA1, and IgA2. The heavy-chain constant regions that correspond to the different classes of immunoglobulins can be α, δ, ε, γ, and μ, respectively. The light chains can be either kappa (or κ) or lambda (or λ).

In some embodiments an antigen binding domain comprises a light chain complementary determining region 1 (LCDR1), a light chain complementary determining region 2 (LCDR2), a light chain complementary determining region 3 (LCDR3), a heavy chain complementary determining region 1 (HCDR1), a heavy chain complementary determining region 2 (HCDR2), and a heavy chain complementary determining region 3 (HCDR3). In some embodiments, an antibody may be a heavy-chain only antibody, in which case the antigen binding domain comprises HCDR1, HCDR2, and HCDR3, and the antibody lacks a light chain. Unless stated otherwise, the CDRs described herein can be defined according to the IMGT (the international ImMunoGeneTics information) system.

An antibody can be chimeric or humanized. Chimeric and humanized forms of non-human (e.g., murine) antibodies can be intact (full length) chimeric immunoglobulins, immunoglobulin chains or antigen binding fragments thereof (such as Fv, Fab, Fab′, F(ab′)₂ or other target-binding subdomains of antibodies), which can contain sequences derived from non-human immunoglobulin. In general, the humanized antibody can comprise substantially all of at least one, and typically two, variable domains, in which all or substantially all of the CDR regions correspond to those of a non-human immunoglobulin and all or substantially all of the framework (FR) regions are those of a human immunoglobulin sequence. A humanized antibody can also comprise at least a portion of an immunoglobulin constant region (Fc), an Fc domain, typically that of a human immunoglobulin sequence.

An antibody described herein can be a human antibody. As used herein, “human antibodies” can include antibodies having, for example, the amino acid sequence of a human immunoglobulin and include antibodies isolated from human immunoglobulin libraries or from animals transgenic for one or more human immunoglobulins and that typically do not express endogenous immunoglobulins. Human antibodies can be produced using transgenic mice which are incapable of expressing functional endogenous immunoglobulins, but which can express human immunoglobulin genes. Completely human antibodies that recognize a selected epitope can be generated using guided selection. In this approach, a selected non-human monoclonal antibody, e.g., a mouse antibody, is used to guide the selection of a completely human antibody recognizing the same epitope

An antibody described herein can be a bispecific antibody or a dual variable domain antibody (DVD). Bispecific and DVD antibodies are monoclonal, often human or humanized, antibodies that have binding specificities for at least two different antigens.

An antibody described herein can be derivatized or otherwise modified. For example, derivatized antibodies can be modified by glycosylation, acetylation, pegylation, phosphorylation, amidation, derivatization by known protecting/blocking groups, proteolytic cleavage, or the like.

An antibody described herein can specifically bind to an antigen that is expressed on a liver cell. For example, an antibody can specifically bind to a liver cell antigen. A liver cell antigen can be expressed on a canalicular cell, Kupffer cell, hepatocyte, a stellate cell, or other liver cell type, or a combination thereof. The liver cell antigen can be a liver cell surface receptor. The liver cell antigen can be a hepatocyte antigen. The liver cell antigen can be expressed on a non-cancerous liver cell. A liver cell antigen can include, but is not limited to, asialoglycoprotein receptor 1 (ASGR1), asialoglycoprotein receptor 2 (ASGR2), transferrin receptor 2 (TRF2), UDP glucuronosyltransferase 1 family, polypeptide A1 (UGT1A1), solute carrier family 22 member 7 (SLC22A7), solute carrier family 13 member 5 (SLC13A5), solute carrier family 22 member 1(SLC22A1), and complement component 9 (C9).

Exemplary Fc Domains

An antibody construct can comprise an Fc domain of an IgG1 isotype. An antibody construct can comprise an Fc domain of an IgG2 isotype. An antibody construct can comprise an Fc domain of an IgG3 isotype. An antibody construct can comprise an Fc domain of an IgG4 isotype. An antibody construct can have a hybrid isotype comprising constant regions from two or more isotypes. An Fc domain typically comprises CH2 and CH3 domains of a heavy chain constant region, but may comprise more or less of the heavy chain constant region as well.

The specificity of the Fc domain to an Fc receptor of a conjugate disclosed herein can be influenced by the presence of a TGFβR1 inhibitor. The Fc domain of the conjugate can bind to an Fc receptor with at least about 10%, about 20%, about 30%, about 40%, about 50%, about 60%, about 70%, about 80%, about 85%, about 90%, about 95%, or about 100% of a specificity of the Fc domain to the Fc receptor in the absence of the TGFβR1 inhibitor.

In some embodiments, an antibody construct includes an Fc domain. The conjugate can comprise an antibody construct comprising an Fc domain that can bind to an FcR when linked to a TGFβR1 inhibitor conjugate. An Fc domain is a structure that can bind to one or more Fc receptors (FcRs). FcRs can bind to an Fc domain of an antibody. FcRs can bind to an Fc domain of an antibody bound to an antigen. FcRs are organized into classes (e.g., gamma (γ), alpha (α) and epsilon (ε)) based on the class of antibody that the FcR recognizes. The FcαR class binds to IgA and includes several isoforms, FcαRI (CD89) and FcαμR. The FcγR class binds to IgG and includes several isoforms, FcγRI (CD64), FcγRIIA (CD32a), FcγRIIB (CD32b), FcγRIIIA (CD16a), and FcγRIIIB (CD16b). An FcγRIIIA (CD16a) can be an FcγRIIIA (CD16a) F158 variant or a V158 variant.

Each FcγR isoform can differ in binding affinity to the Fc domain of the IgG antibody. For example, FcγRI can bind to IgG with greater affinity than FcγRII or FcγRIII. The affinity of a particular FcγR isoform to IgG can be controlled, in part, by a glycan (e.g., oligosaccharide) at position CH2 84.4 of the IgG antibody. For example, fucose containing CH2 84.4 glycans can reduce IgG affinity for FcγRIIIA. In addition, G0 glucans can have increased affinity for FcγRIIIA due to the lack of galactose and terminal GlcNAc moiety.

Binding of an Fc domain to an FcR can enhance an immune response. FcR-mediated signaling that can result from an Fc domain binding to an FcR can lead to the maturation of immune cells. FcR-mediated signaling that can result from an Fc domain binding to an FcR can lead to the maturation of dendritic cells (DCs). FcR-mediated signaling that can result from an Fc domain binding to an FcR can lead to antibody dependent cellular cytotoxicity. FcR-mediated signaling that can result from an Fc domain binding to an FcR can lead to more efficient immune cell antigen uptake and processing. FcR-mediated signaling that can result from an Fc domain binding to an FcR can promote the expansion and activation of T cells. FcR-mediated signaling that can result from an Fc domain binding to an FcR can promote the expansion and activation of CD8+ T cells. FcR-mediated signaling that can result from an Fc domain binding to an FcR can influence immune cell regulation of T cell responses. FcR-mediated signaling that can result from an Fc domain binding to an FcR can influence immune cell regulation of T cell responses. FcR-mediated signaling that can result from an Fc domain binding to an FcR can influence dendritic cell regulation of T cell responses. FcR-mediated signaling that can result from an Fc domain binding to an FcR can influence functional polarization of T cells (e.g., polarization can be toward a TH1 cell response).

The profile of FcRs on a DC can impact the ability of the DC to respond upon stimulation. For example, most DC can express both CD32A and CD32B, which can have opposing effects on IgG-mediated maturation and function of DCs: binding of IgG to CD32A can mature and activate DCs in contrast with CD32B, which can mediate inhibition due to phosphorylation of immunoreceptor tyrosine-based inhibition motif (ITIM), after CD32B binding of IgG. Therefore, the activity of these two receptors can establish a threshold of DC activation. Furthermore, difference in functional avidity of these receptors for IgG can shift their functional balance. Hence, altering the Fc domain binding to FcRs can also shift their functional balance, allowing for manipulation (either enhanced activity or enhanced inhibition) of the DC immune response.

A modification in the amino acid sequence of an Fc domain can alter the recognition of an FcR for the Fc domain. However, such modifications can still allow for FcR-mediated signaling. A modification can be a substitution of an amino acid at a residue of an Fc domain (e.g., wildtype) for a different amino acid at that residue. A modification can permit binding of an FcR to a site on the Fc domain that the FcR may not otherwise bind to. A modification can increase binding affinity of an FcR to the Fc domain. A modification can decrease binding affinity of an FcR to a site on the Fc domain that the FcR may have increased binding affinity for. A modification can increase the subsequent FcR-mediated signaling after Fc domain binding to an FcR.

An Fc domain can be a naturally occurring or a variant of a naturally occurring Fc domain and can comprise at least one amino acid change as compared to the sequence of a wild-type Fc domain. An amino acid change in an Fc domain can allow the antibody or conjugate to bind to at least one Fc receptor with lessor affinity compared to a wild-type Fc domain.

In some embodiments, an Fc domain exhibits increased binding affinity to one or more Fc receptors. In some embodiments, an Fc domain exhibits increased binding affinity to one or more Fcgamma receptors. In some embodiments, an Fc domain exhibits increased binding affinity to FcRn receptors. In some embodiments, an Fc domain exhibits increased binding affinity to Fcgamma and FcRn receptors. In other embodiments, an Fc domain exhibits the same or substantially similar binding affinity to Fcgamma and/or FcRn receptors as compared to a wild-type Fc domain from an IgG antibody (e.g., IgG1 antibody).

In some embodiments, an Fc domain exhibits decreased binding affinity to one or more Fc receptors. In some embodiments, an Fc domain exhibits decreased binding affinity to one or more Fcgamma receptors. In some embodiments, an Fc domain exhibits decreased binding affinity to FcRn receptors. In some embodiments, an Fc domain exhibits decreased binding affinity to Fcgamma and FcRn receptors. In some embodiments, an Fc domain is an Fc null domain. In some embodiments, an Fc domain exhibits decreased binding affinity to FcRn receptors, but exhibits the same or increased binding affinity to one or more Fcgamma receptors as compared to a wildtype Fc domain. In some embodiments, an Fc domain exhibits increased binding affinity to FcRn receptors, but exhibits the same or decreased binding affinity to one or more Fcgamma receptors.

An Fc domain may have one or more, two or more, three or more, or four or more amino acid substitutions that decrease binding of the Fc domain to an Fc receptor. In certain embodiments, an Fc domain has decreased binding affinity for one or more of FcγRI (CD64), FcγRIIA (CD32), FcγRIIIA (CD16a), FcγRIIIB (CD16b), or any combination thereof. In order to decrease binding affinity of an Fc domain to an Fc receptor, the Fc domain may comprise one or more amino acid substitutions that reduces the binding affinity of the Fc domain to an Fc receptor. In other embodiments, an Fc domain exhibits the same or substantially similar binding affinity to one or more of FcγRI (CD64), FcγRIIA (CD32), FcγRIIIA (CD16a), FcγRIIIB (CD16b), or any combination thereof as compared to a wild-type Fc domain from an IgG antibody (e.g., IgG1 antibody). In some embodiments, an Fc domain can comprise a sequence of an IgG isoform that has been modified from the wild-type IgG sequence. In some embodiments, the Fc domain can comprise a sequence of the IgG1 isoform that has been modified from the wild-type IgG1 sequence. In some embodiments, the modification comprises substitution of one or more amino acids that reduce binding affinity of an IgG Fc domain to all Fc receptors.

A modification can be substitution of E233, L234 and L235, such as E233P/L234V/L235A or E233P/L234V/L235A/ΔG236, according to the EU index of Kabat. A modification can be a substitution of P238, such as P238A, according to the EU index of Kabat. A modification can be a substitution of D265, such as D265A, according to the EU index of Kabat. A modification can be a substitution of N297, such as N297A, according to the EU index of Kabat. A modification can be a substitution of A327, such as A327Q, according to the EU index of Kabat. A modification can be a substitution of P329, such as P239A, according to the EU index of Kabat.

In some embodiments, an IgG Fc domain comprises at least one amino acid substitution that reduces its binding affinity to FcγR1, as compared to a wild-type or reference IgG Fc domain. A modification can comprise a substitution at F241, such as F241A, according to the EU index of Kabat. A modification can comprise a substitution at F243, such as F243A, according to the EU index of Kabat. A modification can comprise a substitution at V264, such as V264A, according to the EU index of Kabat. A modification can comprise a substitution at D265, such as D265A according to the EU index of Kabat.

In some embodiments, an IgG Fc domain comprises at least one amino acid substitution that increases its binding affinity to FcγR1, as compared to a wild-type or reference IgG Fc domain. A modification can comprise a substitution at A327 and P329, such as A327Q/P329A, according to the EU index of Kabat.

In some embodiments, the modification comprises substitution of one or more amino acids that reduce binding affinity of an IgG Fc domain to FcγRII and FcγRIIIA receptors. A modification can be a substitution of D270, such as D270A, according to the EU index of Kabat. A modification can be a substitution of Q295, such as Q295A, according to the EU index of Kabat. A modification can be a substitution of A327, such as A237S, according to the EU index of Kabat.

In some embodiments, the modification comprises substitution of one or more amino acids that increases binding affinity of an IgG Fc domain to FcγRII and FcγRIIIA receptors. A modification can be a substitution of T256, such as T256A, according to the EU index of Kabat. A modification can be a substitution of K290, such as K290A, according to the EU index of Kabat.

In some embodiments, the modification comprises substitution of one or more amino acids that increases binding affinity of an IgG Fc domain to FcγRII receptor. A modification can be a substitution of R255, such as R255A, according to the EU index of Kabat. A modification can be a substitution of E258, such as E258A, according to the EU index of Kabat. A modification can be a substitution of S267, such as S267A, according to the EU index of Kabat. A modification can be a substitution of E272, such as E272A, according to the EU index of Kabat. A modification can be a substitution of N276, such as N276A, according to the EU index of Kabat. A modification can be a substitution of D280, such as D280A, according to the EU index of Kabat. A modification can be a substitution of H285, such as H285A, according to the EU index of Kabat. A modification can be a substitution of N286, such as N286A, according to the EU index of Kabat. A modification can be a substitution of T307, such as T307A, according to the EU index of Kabat. A modification can be a substitution of L309, such as L309A, according to the EU index of Kabat. A modification can be a substitution of N315, such as N315A, according to the EU index of Kabat. A modification can be a substitution of K326, such as K326A, according to the EU index of Kabat. A modification can be a substitution of P331, such as P331A, according to the EU index of Kabat. A modification can be a substitution of S337, such as S337A, according to the EU index of Kabat. A modification can be a substitution of A378, such as A378A, according to the EU index of Kabat. A modification can be a substitution of E430, such as E430, according to the EU index of Kabat.

An amino acid change in an Fc domain can allow the antibody construct or conjugate to bind to at least one Fc receptor with greater affinity compared to a wild-type Fe domain. An Fc domain variant can comprise an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. An Fc domain variant can comprise a sequence of the IgG1 isoform that has been modified from an wildtype IgG1 sequence to increase Fc receptor binding.

In some embodiments, the modification comprises substitution of one or more amino acids that increases binding affinity of an IgG Fc domain to FcγRII receptor and reduces the binding affinity to FcγRIIIA receptor. A modification can be a substitution of H268, such as H268A, according to the EU index of Kabat. A modification can be a substitution of R301, such as R301A, according to the EU index of Kabat. A modification can be a substitution of K322, such as K322A, according to the EU index of Kabat.

In some embodiments, the modification comprises substitution of one or more amino acids that decreases binding affinity of an IgG Fc domain to FcγRII receptor but does not affect the binding affinity to FcγRIIIA receptor. A modification can be a substitution of R292, such as R292A, according to the EU index of Kabat. A modification can be a substitution of K414, such as K414A, according to the EU index of Kabat.

In some embodiments, the modification comprises substitution of one or more amino acids that decreases binding affinity of an IgG Fc domain to FcγRII receptor and increases the binding affinity to FcγRIIIA receptor. A modification can be a substitution of S298, such as S298A, according to the EU index of Kabat. A modification can be substitution of S239, I332 and A330, such as S239D/I332E/A330L. A modification can be substitution of S239 and I332, such as S239D/I332E.

In some embodiments, the modification comprises substitution of one or more amino acids that decreases binding affinity of an IgG Fc domain to FcγRIIIA receptor. A modification can be substitution of F241 and F243, such as F241S/F243S or F241I/F243I, according to the EU index of Kabat.

In some embodiments, the modification comprises substitution of one or more amino acids that decreases binding affinity of an IgG Fc domain to FcγRIIIA receptor and does not affect the binding affinity to FcγRII receptor. A modification can be a substitution of S239, such as S239A, according to the EU index of Kabat. A modification can be a substitution of E269, such as E269A, according to the EU index of Kabat. A modification can be a substitution of E293, such as E293A, according to the EU index of Kabat. A modification can be a substitution of Y296, such as Y296F, according to the EU index of Kabat. A modification can be a substitution of V303, such as V303A, according to the EU index of Kabat. A modification can be a substitution of A327, such as A327G, according to the EU index of Kabat. A modification can be a substitution of K338, such as K338A, according to the EU index of Kabat. A modification can be a substitution of D376, such as D376A, according to the EU index of Kabat.

In some embodiments, the modification comprises substitution of one or more amino acids that increases binding affinity of an IgG Fc domain to FcγRIIIA receptor and does not affect the binding affinity to FcγRII receptor. A modification can be a substitution of E333, such as E333A, according to the EU index of Kabat. A modification can be a substitution of K334, such as K334A, according to the EU index of Kabat. A modification can be a substitution of A339, such as A339T, according to the EU index of Kabat. A modification can be substitution of S239 and I332, such as S239D/I332E.

In some embodiments, the modification comprises substitution of one or more amino acids that increases binding affinity of an IgG Fc domain to FcγRIIIA receptor. A modification can be substitution of L235, F243, R292, Y300 and P396, such as L235V/F243L/R292P/Y300L/P396L (IgG1VLPLL) according to the EU index of Kabat. A modification can be substitution of S298, E333 and K334, such as S298A/E333A/K334A, according to the EU index of Kabat. A modification can be substitution of K246, such as K246F, according to the EU index of Kabat.

Other substitutions in an IgG Fc domain that affect its interaction with one or more Fcγ receptors are disclosed in U.S. Pat. Nos. 7,317,091 and 8,969,526 (the disclosures of which are incorporated by reference herein).

In some embodiments, an IgG Fc domain comprises at least one amino acid substitution that reduces the binding affinity to FcRn, as compared to a wild-type or reference IgG Fc domain. A modification can comprise a substitution at H435, such as H435A according to the EU index of Kabat. A modification can comprise a substitution at I253, such as I253A according to the EU index of Kabat. A modification can comprise a substitution at H310, such as H310A according to the EU index of Kabat. A modification can comprise substitutions at I253, H310 and H435, such as I253A/H310A/H435A according to the EU index of Kabat.

A modification can comprise a substitution of one amino acid residue that increases the binding affinity of an IgG Fc domain for FcRn, relative to a wildtype or reference IgG Fc domain. A modification can comprise a substitution at V308, such as V308P according to the EU index of Kabat. A modification can comprise a substitution at M428, such as M428L according to the EU index of Kabat. A modification can comprise a substitution at N434, such as N434A according to the EU index of Kabat or N434H according to the EU index of Kabat. A modification can comprise substitutions at T250 and M428, such as T250Q and M428L according to the EU index of Kabat. A modification can comprise substitutions at M428 and N434, such as M428L and N434S, N434A or N434H according to the EU index of Kabat. A modification can comprise substitutions at M252, S254 and T256, such as M252Y/S254T/T256E according to the EU index of Kabat. A modification can be a substitution of one or more amino acids selected from P257L, P257N, P257I, V279E, V279Q, V279Y, A281S, E283F, V284E, L306Y, T307V, V308F, Q311V, D376V, and N434H. Other substitutions in an IgG Fc domain that affect its interaction with FcRn are disclosed in U.S. Pat. No. 9,803,023 (the disclosure of which is incorporated by reference herein).

In some embodiments, an antibody is a human IgG2 antibody, including an IgG2 Fc region. In some embodiments, the heavy chain of the human IgG2 antibody can be mutated at cysteines as positions 127, 232, or 233. In some embodiments, the light chain of a human IgG2 antibody can be mutated at a cysteine at position 214. The mutations in the heavy and light chains of the human IgG2 antibody can be from a cysteine residue to a serine residue.

A modification can comprise a substitution at one or more one amino acid residues of an Fc domain such as at 5 different amino acid residues including L235V/F243L/R292P/Y300L/P396L (IgG1VLPLL). The numbering of amino acids residues described herein is according to the EU index. This modification can be located in a portion of an antibody construct, which can include an Fc domain and, in particular, can be located in a portion of the Fc domain that can bind to Fc receptors. A modification can comprise a substitution at one or more amino acid residues such as at 2 different amino acid residues of an Fc domain, including S239D/I332E (IgG1DE). This modification can be located in a portion of an antibody sequence which includes an Fc domain of the antibody and in particular, are located in portions of the Fc domain that can bind to Fc receptors. A modification can comprise a substitution at one or more amino acid residues such as at 3 different amino acid residues of an Fc domain including S298A/E333A/K334A (IgG1AAA). The modification can be located in a portion of an antibody sequence, which includes an Fc domain of the antibody and, in particular, can be located in portions of the Fc domain that can bind Fc receptors.

Binding of Fc receptors to an Fc domain can be affected by amino acid substitutions. For example, binding of some Fc receptors to an Fc domain variant comprising the IgG1VLPLL modifications can be enhanced compared to wild-type by as result of the L235V/F243L/R292P/Y300L/P396L amino acid modifications. However, binding of other Fc receptors to the Fc domain variant comprising the IgG1VLPLL modifications can be reduced compared to wild-type by the L235V/F243L/R292P/Y300L/P396L amino acid modifications. For example, the binding affinities of the Fc domain variant comprising the IgG1VLPLL modifications to FcγRIIIA and to FcγRIIA can be enhanced compared to wild-type whereas the binding affinity of the Fc domain variant comprising the IgG1VLPLL modifications to FcγRIIB can be reduced compared to wild-type. Binding of Fc receptors to an Fc domain variant comprising the IgG1DE modifications can be enhanced compared to wild-type as a result of the S239D/I332E amino acid modification. However, binding of some Fc receptors to the Fc domain variant comprising the IgG1DE modifications can be reduced compared to wild-type by S239D/I332E amino acid modification. For example, the binding affinities of the Fc domain variant comprising the IgG1DE modifications to FcγRIIIA and to FcγRIIB can be enhanced compared to wild-type. Binding of Fc receptors to an Fc domain variant comprising the IgG1AAA modifications can be enhanced compared to wild-type as a result of the S298A/E333A/K334A amino acid modification. However, binding of some Fc receptors to Fc domain variant comprising the IgG1AAA modifications can be reduced compared to wild-type by S298A/E333A/K334A amino acid modification. Binding affinities of the Fc domain variant comprising the IgG1AAA modifications to FcγRIIIA can be enhanced compared to wild-type whereas the binding affinity of the Fc domain variant comprising the IgG1AAA modifications to FcγRIIB can be reduced compared to wildtype.

In some embodiments, the heavy chain of a human IgG2 antibody can be mutated at cysteines as positions 127, 232, or 233. In some embodiments, the light chain of a human IgG2 antibody can be mutated at a cysteine at position 214. The mutations in the heavy and light chains of the human IgG2 antibody can be from a cysteine residue to a serine residue.

While an antibody construct can comprise a first binding domain and a second binding domain with wild-type or modified amino acid sequences encoding the Fc domain, the modifications of the Fc domain from the wild-type sequence may not significantly alter binding and/or affinity of the Fc domain or the antigen binding domain(s). For example, binding and/or affinity of an antibody construct comprising a first binding domain and a second binding domain (or, in some cases, a third binding domain) and having the Fc domain modifications of IgG1VLPLL, IgG1DE, or IgG1AAA may not be significantly altered by modification of an Fc domain amino acid sequence compared to a wild-type sequence. Modifications of an Fc domain from a wild-type sequence may not alter binding and/or affinity of a first binding domain or target binding domain that binds, for example, to a liver cell antigen or a viral antigen from a virus infecting liver cell. Additionally, the binding and/or affinity of the binding domains described herein, for example a first binding domain, a second binding domain (or, in some cases, a third binding domain), and an Fc domain variant selected from IgG1VLPLL, IgG1DE, and IgG1AAA, may be comparable to the binding and/or affinity of wild-type antibodies.

An Fc domain can be from an antibody. An Fc domain can be from an IgG antibody. An Fc domain can be from an IgG1, IgG2, or IgG4 antibody. An Fc domain can be at least 80% identical to an Fc domain from an antibody. An Fc domain can be a portion of the Fc domain of an antibody.

An antibody construct can comprise an Fc domain in an antibody. An antibody construct can comprise an Fc domain in a scaffold. An antibody construct can comprise an Fc domain in an antibody scaffold. An antibody construct can comprise an Fc domain in a non-antibody scaffold. An antibody construct can comprise an Fc domain covalently attached to an antigen binding domain.

An antibody construct can comprise an antigen binding domain and an Fc domain, wherein the Fc domain is covalently attached to the antigen binding domain. An antibody construct can comprise an antigen binding domain and Fc domain, wherein the Fc domain is covalently attached to the antigen binding domain as an Fc domain-antigen binding domain fusion protein. An antibody construct can comprise an antigen binding domain and Fc domain, wherein the Fc domain is covalently attached to the antigen binding domain by a linker.

The conjugate can comprise an Fc domain that can bind to an FcR to initiate FcR-mediated signaling when linked to a TGFβR1 inhibitor conjugate. The conjugate can bind to ASGR1 or ASGR2 when linked to a TGFβR1 inhibitor. The conjugate can bind to ASGR1 or ASGR2 when linked to a TGFβR1 inhibitor conjugate and the Fc domain of the conjugate can bind to an FcR when linked to a TGFβR1 inhibitor. The conjugate can bind to ASGR1 or ASGR2 when linked to a TGFβR1 inhibitor conjugate and the Fc domain of the conjugate can bind to an FcR to initiate FcR-mediated signaling when linked to a TGFβR1 inhibitor conjugate. The Fc domain linked to a TGFβR1 inhibitor conjugate can be a Fc domain variant. The Fc domain variant can comprise a substitution at more than one amino acid residue, such as at 5 different amino acid residues including L235V/F243L/R292P/Y300L/P396L, as at 2 different amino acid residues including S239D/I332E, or as at 3 different amino acid residues including S298A/E333A/K334A.

The Kd for binding of an Fc domain to a Fc receptor when the Fc domain is linked to a TGFβR1 inhibitor conjugate can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the Kd for binding of the Fc domain to the Fc receptor in the absence of linking to the TGFβR1 inhibitor conjugate. The Kd for binding of an Fc domain to an Fc receptor when linked to a TGFβR1 inhibitor can be less than 10 nM. The Kd for binding of an Fc domain to an Fc receptor when linked to a TGFβR1 inhibitor conjugate can be less than 100 nM, less than 50 nM, less than 20 nM, less than 5 nM, less than 1 nM, or less than 0.1 nM. In contrast, the Kd for binding of an Fc domain to an Fc receptor when linked to a TGFβR1 inhibitor conjugate and when the first binding domain is bound to its antigen can be greater than 100 nM. The Kd for binding of an Fc domain to an Fc receptor when linked to a TGFβR1 inhibitor conjugate and when the first binding domain is bound to its antigen can be greater than 100 nM, greater than 200 nM, greater than 300 nM, greater than 400 nM, greater than 500 nM, or greater than 1000 nM.

The affinity of the Fc domain to an Fc receptor of a conjugate disclosed herein can be influenced by the presence of a TGFβR1 inhibitor. The Fc domain of the conjugate can bind to an Fc receptor with at least about 1%, about 5%, about 10%, about 20%, about 30%, about 40%, about 50%, about 60%, about 70%, about 80%, about 85%, about 90%, about 95%, or about 100% of an affinity of the Fc domain to the Fc receptor in the absence of the TGFβR1 inhibitor.

Anti-ASGR1 Antibodies and Binding Domains Thereof The present disclosure provides exemplary anti-ASGR1 antibodies (i.e., antibodies, such as isolated monoclonal antibodies, that specifically bind to ASGR1) and their ASGR1-binding domains thereof that may be used in forming TGFβR1 inhibitor-ASGR antibody conjugates. In certain embodiments, such an anti-ASGR1 antibody is linked with a TGFβR1 inhibitor to form an anti-ASGR1 antibody TGFβR1 inhibitor conjugate. In certain other embodiments, an exemplary ASGR1-binding domain provided herein and a second antigen binding domain form part of a bispecific antibody construct, which in turn is used in forming a TGFβR1 inhibitor-bispecific antibody conjugate. In any of the aforementioned antibodies specific for ASGR1 are specific for human ASGR1.

As demonstrated in the Examples provided herein, certain exemplary anti-ASGR1 antibodies (e.g., H8K and L4L) of the present disclosure were determined to belong to a unique epitope bin compared to reference anti-ASGR1 antibodies and block ASGR1 ligand (GalNac) binding to ASGR1. While certain exemplary anti-ASGR1 antibodies (e.g., G2D, J4F, and K2E) belonged to the same epitope bin as a reference anti-ASGR1 antibody, G2D and K2E antibodies unexpectedly differ from the reference anti-ASGR1 antibody in that: they do not exhibit off-target binding to rat CLEC10A whereas the reference antibody does; and the G2D antibody does not compete with ASGR1 ligand (GalNac) binding and K2E antibody only minimally blocks GalNac binding, whereas the reference antibody competes with GalNac binding. In addition, certain exemplary anti-ASGR1 antibodies exhibited calcium sensitivity in binding with ASGR1 (e.g., during association and/or disassociation with ASGR1). The Examples provided herein also show that addition of a G27Y mutation in the VH region of certain exemplary humanized anti-ASGR1 antibodies stabilizes the antibody structure, resulting in more homogenous recovery.

In various embodiments, an antibody or antigen binding fragment thereof comprises two light chain polypeptides (light chains) and two heavy chain polypeptides (heavy chains), held together covalently by disulfide linkages.

The heavy chain typically comprises a heavy chain variable region (VH) and a heavy chain constant region. The heavy chain constant region typically comprises three domains, CH1, CH2, and CH3. Non-limiting exemplary heavy chain constant regions include human IgG1, human IgG2, human IgG3, and human IgG4 constant regions. In some embodiments, an antibody provided herein comprises an IgG1 constant region. Exemplary heavy chain constant regions include human IgG1 heavy chain constant region (SEQ ID NO:230), human IgG1 null heavy chain constant region (SEQ ID NO:231), mouse IgG2a heavy chain constant region (SEQ ID NO:233), and rat IgG2b heavy chain constant region (SEQ ID NO:235).

The light chain typically comprises a light chain variable region (VL) and a light chain constant region. Non-limiting exemplary light chain constant regions include kappa and lambda constant regions. A non-limiting exemplary human kappa constant region is shown in SEQ ID NO:232. Another exemplary light chain constant region is mouse kappa constant region shown in SEQ ID NO:234. Another exemplary light chain constant region is rat kappa constant region shown in SEQ ID NO:236.

The exemplary anti-ASGR1 antibodies provided herein may comprise Fc domains. Exemplary Fc domains are described above. Similarly, the exemplary anti-ASGR1 binding domains provided herein may be linked to Fc domains as described above.

In certain embodiments, an Fc region or domain found in an exemplary anti-ASGR1 antibody provided herein will be capable of mediating one or more of these effector functions, or will lack one or more or all of these activities or have one or more of the effector activities increased by way of, for example, one or more mutations as compared to the unmodified Fc region or domain.

In some embodiments, an anti-ASGR1 antibody or antigen binding fragment thereof comprises:
a heavy chain CDR1 (VH-CDR1) comprising the amino acid sequence of SEQ ID NO:1, a VH-CDR2 comprising the amino acid sequence selected from any one of SEQ ID NOS:6-8, a VH-CDR3 comprising the amino acid sequence of SEQ ID NO:13; and alight chain CDR1 (VL-CDR1) comprising the amino acid sequence of SEQ ID NO:18, a VL-CDR2 comprising the amino acid sequence selected from any one of SEQ ID NOS:23-25, and a VL-CDR3 comprising the amino acid sequence of SEQ ID NO:33;
a VH-CDR1 comprising the amino acid sequence of SEQ ID NO:2, a VH-CDR2 comprising the amino acid sequence of SEQ ID NO:9, a VH-CDR3 comprising the amino acid sequence of SEQ ID NO:14; and a VL-CDR1 comprising the amino acid sequence of SEQ ID NO:19, a VL-CDR2 comprising the amino acid sequence of SEQ ID NO:26 or SEQ ID NO:27, and a VL-CDR3 comprising the amino acid sequence of SEQ ID NO:34;
a VH-CDR1 comprising the amino acid sequence of SEQ ID NO:5, a VH-CDR2 comprising the amino acid sequence of SEQ ID NO:12, a VH-CDR3 comprising the amino acid sequence of SEQ ID NO:17; and a VL-CDR1 comprising the amino acid sequence of SEQ ID NO:22, a VL-CDR2 comprising the amino acid sequence of SEQ ID NO:32, and a VL-CDR3 comprising the amino acid sequence of SEQ ID NO:37;
a VH-CDR1 comprising the amino acid sequence of SEQ ID NO:3, a VH-CDR2 comprising the amino acid sequence of SEQ ID NO:10, a VH-CDR3 comprising the amino acid sequence of SEQ ID NO:15; and a VL-CDR1 comprising the amino acid sequence of SEQ ID NO:20, a VL-CDR2 comprising the amino acid sequence selected from any one of SEQ ID NOS:28-30, and a VL-CDR3 comprising the amino acid sequence of SEQ ID NO:35; or
a VH-CDR1 comprising the amino acid sequence of SEQ ID NO:4, a VH-CDR2 comprising the amino acid sequence of SEQ ID NO:11, a VH-CDR3 comprising the amino acid sequence of SEQ ID NO:16; and a VL-CDR1 comprising the amino acid sequence of SEQ ID NO:21, a VL-CDR2 comprising the amino acid sequence of SEQ ID NO:31, and a VL-CDR3 comprising the amino acid sequence of SEQ ID NO:36.

In some such embodiments, the antibody or antigen binding fragment thereof comprises a CDR1 (VH-CDR1) comprising the amino acid sequence of SEQ ID NO:1, a VH-CDR2 comprising the amino acid sequence selected from any one of SEQ ID NO:8, a VH-CDR3 comprising the amino acid sequence of SEQ ID NO:13; and the VL comprises a CDR1 (VL-CDR1) comprising the amino acid sequence of SEQ ID NO:18, a VL-CDR2 comprising the amino acid sequence selected from any one of SEQ ID NO:23, and a VL-CDR3 comprising the amino acid sequence of SEQ ID NO:33.

In some such embodiments, the antibody or antigen binding fragment thereof comprises a CDR1 (VH-CDR1) comprising the amino acid sequence of SEQ ID NO:1, a VH-CDR2 comprising the amino acid sequence selected from any one of SEQ ID NO:6, a VH-CDR3 comprising the amino acid sequence of SEQ ID NO:13; and the VL comprises a CDR1 (VL-CDR1) comprising the amino acid sequence of SEQ ID NO:18, a VL-CDR2 comprising the amino acid sequence selected from any one of SEQ ID NO:23, and a VL-CDR3 comprising the amino acid sequence of SEQ ID NO:33.

In some such embodiments, the antibody or antigen binding fragment thereof comprises a CDR1 (VH-CDR1) comprising the amino acid sequence of SEQ ID NO:2, a VH-CDR2 comprising the amino acid sequence selected from any one of SEQ ID NO:9, a VH-CDR3 comprising the amino acid sequence of SEQ ID NO:14; and the VL comprises a CDR1 (VL-CDR1) comprising the amino acid sequence of SEQ ID NO:19, a VL-CDR2 comprising the amino acid sequence selected from any one of SEQ ID NO:26, and a VL-CDR3 comprising the amino acid sequence of SEQ ID NO:34.

In some such embodiments, the antibody or antigen binding fragment thereof comprises a heavy chain variable region (VH) comprising an amino acid sequence that is at least 90%, 91%, 92%, 93%, 94%, 95%, 96%, 97%, 98%, or 99% identical to the amino acid sequence of SEQ ID NO:38, and a light chain variable region (VL) comprising an amino acid sequence that is at least 90%, 91%, 92%, 93%, 94%, 95%, 96%, 97%, 98%, or 99% identical to the amino acid sequence of SEQ ID NO:126, provided that the amino acid sequences of the VH-CDRs (i.e., SEQ ID NOS:1, 6, and 13) and VL-CDRs (i.e., SEQ ID NOS:18, 23, and 33) are unchanged.

In some such embodiments, the antibody or antigen binding fragment thereof comprises a heavy chain variable region (VH) comprising an amino acid sequence that is at least 90%, 91%, 92%, 93%, 94%, 95%, 96%, 97%, 98%, or 99% identical to the amino acid sequence selected from any one of SEQ ID NOS:43-88 and 238-243, and a light chain variable region (VL) comprising an amino acid sequence that is at least 90%, 91%, 92%, 93%, 94%, 95%, 96%, 97%, 98%, or 99% identical to the amino acid sequence selected from any one of SEQ ID NOS:131-133 and 244-249, provided that the amino acid sequences of the VH-CDRs and VL-CDRs are unchanged.

In certain embodiments, the antibody or antigen binding fragment thereof comprises a heavy chain variable region (VH) comprising an amino acid sequence that is at least 90%, 91%, 92%, 93%, 94%, 95%, 96%, 97%, 98%, or 99% identical to the amino acid sequence of SEQ ID NO:240, and a light chain variable region (VL) comprising an amino acid sequence that is at least 90%, 91%, 92%, 93%, 94%, 95%, 96%, 97%, 98%, or 99% identical to the amino acid sequence of SEQ ID NO:247, provided that the amino acid sequences of the VH-CDRs and VL-CDRs are unchanged. In further embodiments, the antibody or antigen binding fragment thereof comprises a heavy chain variable region (VH) comprising an amino acid sequence that is at least 90%, 91%, 92%, 93%, 94%, 95%, 96%, 97%, 98%, or 99% identical to the amino acid sequence of SEQ ID NO:239, and a light chain variable region (VL) comprising an amino acid sequence that is at least 90%, 91%, 92%, 93%, 94%, 95%, 96%, 97%, 98%, or 99% identical to the amino acid sequence of SEQ ID NO:247, provided that the amino acid sequences of the VH-CDRs and VL-CDRs are unchanged.

In certain embodiments, the antibody or antigen binding fragment thereof comprises a heavy chain variable region (VH) comprising an amino acid sequence that is at least 90%, 91%, 92%, 93%, 94%, 95%, 96%, 97%, 98%, or 99% identical to the amino acid sequence of SEQ ID NO:240, and a light chain variable region (VL) comprising an amino acid sequence that is at least 90%, 91%, 92%, 93%, 94%, 95%, 96%, 97%, 98%, or 99% identical to the amino acid sequence of SEQ ID NO:248, provided that the amino acid sequences of the VH-CDRs and VL-CDRs are unchanged. In further embodiments, the antibody or antigen binding fragment thereof comprises a heavy chain variable region (VH) comprising an amino acid sequence that is at least 90%, 91%, 92%, 93%, 94%, 95%, 96%, 97%, 98%, or 99% identical to the amino acid sequence of SEQ ID NO:239, and a light chain variable region (VL) comprising an amino acid sequence that is at least 90%, 91%, 92%, 93%, 94%, 95%, 96%, 97%, 98%, or 99% identical to the amino acid sequence of SEQ ID NO:248, provided that the amino acid sequences of the VH-CDRs and VL-CDRs are unchanged.

In some embodiments, the antibody or antigen binding fragment thereof comprises a heavy chain comprising an amino acid sequence that is at least 90%, 91%, 92%, 93%, 94%, 95%, 96%, 97%, 98%, or 99% identical to the amino acid sequence selected from SEQ ID NO:39, and a light chain comprising an amino acid sequence that is at least 90%, 91%, 92%, 93%, 94%, 95%, 96%, 97%, 98%, or 99% identical to the amino acid sequence of SEQ ID NO:127, provided that the amino acid sequences of the VH-CDRs (i.e., SEQ ID NOS:2, 9, and 14) and VL-CDRs (i.e., SEQ ID NOS:19, 26, and 34) are unchanged.

In some such embodiments, the antibody or antigen binding fragment thereof comprises a heavy chain variable region (VH) comprising an amino acid sequence that is at least 90%, 91%, 92%, 93%, 94%, 95%, 96%, 97%, 98%, or 99% identical to the amino acid sequence selected from any one of SEQ ID NOS:89-93, and a light chain variable region (VL) comprising an amino acid sequence that is at least 90%, 91%, 92%, 93%, 94%, 95%, 96%, 97%, 98%, or 99% identical to the amino acid sequence selected from any one of SEQ ID NOS:134-137, provided that the amino acid sequences of the VH-CDRs and VL-CDRs are unchanged.

In certain embodiments, the antibody or antigen binding fragment thereof comprises a heavy chain variable region (VH) comprising an amino acid sequence that is at least 90%, 91%, 92%, 93%, 94%, 95%, 96%, 97%, 98%, or 99% identical to the amino acid sequence of SEQ ID NO:89, and a light chain variable region (VL) comprising an amino acid sequence that is at least 90%, 91%, 92%, 93%, 94%, 95%, 96%, 97%, 98%, or 99% identical to the amino acid sequence of SEQ ID NO:134, provided that the amino acid sequences of the VH-CDRs and VL-CDRs are unchanged. In further embodiments, the antibody or antigen binding fragment thereof comprises a heavy chain variable region (VH) comprising an amino acid sequence that is at least 90%, 91%, 92%, 93%, 94%, 95%, 96%, 97%, 98%, or 99% identical to the amino acid sequence of SEQ ID NO:89, and a light chain variable region (VL) comprising an amino acid sequence that is at least 90%, 91%, 92%, 93%, 94%, 95%, 96%, 97%, 98%, or 99% identical to the amino acid sequence of SEQ ID NO:136, provided that the amino acid sequences of the VH-CDRs and VL-CDRs are unchanged.

In some such embodiments, the antibody or antigen binding fragment thereof comprises a heavy chain variable region (VH) comprising an amino acid sequence that is at least 90%, 91%, 92%, 93%, 94%, 95%, 96%, 97%, 98%, or 99% identical to the amino acid sequence of SEQ ID NO:42, and a light chain variable region (VL) comprising an amino acid sequence that is at least 90%, 91%, 92%, 93%, 94%, 95%, 96%, 97%, 98%, or 99% identical to the amino acid sequence of SEQ ID NO:130, provided that the amino acid sequences of the VH-CDRs (i.e., SEQ ID NOS:5, 12, and 17) and VL-CDRs (i.e., SEQ ID NOS:22, 32, and 37) are unchanged.

In some such embodiments, the antibody or antigen binding fragment thereof comprises a heavy chain variable region (VH) comprising an amino acid sequence that is at least 90%, 91%, 92%, 93%, 94%, 95%, 96%, 97%, 98%, or 99% identical to the amino acid sequence selected from any one of SEQ ID NOS:113-125, and a light chain variable region (VL) comprising an amino acid sequence that is at least 90%, 91%, 92%, 93%, 94%, 95%, 96%, 97%, 98%, or 99% identical to the amino acid sequence selected from any one of SEQ ID NOS:143-149, provided that the amino acid sequences of the VH-CDRs and VL-CDRs are unchanged.

In some such embodiments, the antibody or antigen binding fragment thereof comprises a heavy chain variable region (VH) comprising an amino acid sequence that is at least 90%, 91%, 92%, 93%, 94%, 95%, 96%, 97%, 98%, or 99% identical to the amino acid sequence of SEQ ID NO:40, and a light chain variable region (VL) comprising an amino acid sequence that is at least 90%, 91%, 92%, 93%, 94%, 95%, 96%, 97%, 98%, or 99% identical to the amino acid sequence of SEQ ID NO:128, provided that the amino acid sequences of the VH-CDRs (i.e., SEQ ID NOS:3, 10, and 15) and VL-CDRs (i.e., SEQ ID NOS:20, 28, and 35) are unchanged.

In some such embodiments, the antibody or antigen binding fragment thereof comprises a heavy chain variable region (VH) comprising an amino acid sequence that is at least 90%, 91%, 92%, 93%, 94%, 95%, 96%, 97%, 98%, or 99% identical to the amino acid sequence selected from any one of SEQ ID NOS:94-102, and a light chain variable region (VL) comprising an amino acid sequence that is at least 90%, 91%, 92%, 93%, 94%, 95%, 96%, 97%, 98%, or 99% identical to the amino acid sequence selected from any one of SEQ ID NOS:138-141, provided that the amino acid sequences of the VH-CDRs and VL-CDRs are unchanged.

In some such embodiments, the antibody or antigen binding fragment thereof comprises a heavy chain variable region (VH) comprising an amino acid sequence that is at least 90%, 91%, 92%, 93%, 94%, 95%, 96%, 97%, 98%, or 99% identical to the amino acid sequence of SEQ ID NO:41, and a light chain variable region (VL) comprising an amino acid sequence that is at least 90%, 91%, 92%, 93%, 94%, 95%, 96%, 97%, 98%, or 99% identical to the amino acid sequence of SEQ ID NO:129, provided that the amino acid sequences of the VH-CDRs (i.e., SEQ ID NOS:4, 11, and 16) and VL-CDRs (i.e., SEQ ID NOS:21, 31, and 36) are unchanged.

In some such embodiments, the antibody or antigen binding fragment thereof comprises a heavy chain variable region (VH) comprising an amino acid sequence that is at least 90%, 91%, 92%, 93%, 94%, 95%, 96%, 97%, 98%, or 99% identical to the amino acid sequence selected from any one of SEQ ID NOS:103-112, and a light chain variable region (VL) comprising an amino acid sequence that is at least 90%, 91%, 92%, 93%, 94%, 95%, 96%, 97%, 98%, or 99% identical to the amino acid sequence of SEQ ID NO:142, provided that the amino acid sequences of the VH-CDRs and VL-CDRs are unchanged.

In some such embodiments, the antibody or antigen binding fragment thereof comprises a heavy chain variable region (VH) comprising an amino acid sequence of SEQ ID NO:38, and a light chain variable region (VL) comprising an amino acid sequence of SEQ ID NO:126.

In some such embodiments, the antibody or antigen binding fragment thereof comprises a VH comprising an amino acid sequence selected from any one of SEQ ID NOS:43-88 and 238-243, and a VL comprising an amino acid sequence selected from any one of SEQ ID NOS:131-133 and 244-249.

In some such embodiments, the antibody or antigen binding fragment thereof comprises a VH comprising the amino acid sequence of SEQ ID NO:43 and a VL comprising the amino acid sequence of SEQ ID NO:131.

In some such embodiments, the antibody or antigen binding fragment thereof comprises a VH comprising the amino acid sequence of SEQ ID NO:44 and a VL comprising the amino acid sequence of SEQ ID NO:131.

In some such embodiments, the antibody or antigen binding fragment thereof comprises a VH comprising the amino acid sequence of SEQ ID NO:45 and a VL comprising the amino acid sequence of SEQ ID NO:131.

In some such embodiments, the antibody or antigen binding fragment thereof comprises a VH comprising the amino acid sequence of SEQ ID NO:46 and a VL comprising the amino acid sequence of SEQ ID NO:131.

In some such embodiments, the antibody or antigen binding fragment thereof comprises a VH comprising the amino acid sequence of SEQ ID NO:47 and a VL comprising the amino acid sequence of SEQ ID NO:131.

In some such embodiments, the antibody or antigen binding fragment thereof comprises a VH comprising the amino acid sequence of SEQ ID NO:48 and a VL comprising the amino acid sequence of SEQ ID NO:131.

In some such embodiments, the antibody or antigen binding fragment thereof comprises a VH comprising the amino acid sequence of SEQ ID NO:49 and a VL comprising the amino acid sequence of SEQ ID NO:131.

In some such embodiments, the antibody or antigen binding fragment thereof comprises a VH comprising the amino acid sequence of SEQ ID NO:50 and a VL comprising the amino acid sequence of SEQ ID NO:131.

In some such embodiments, the antibody or antigen binding fragment thereof comprises a VH comprising the amino acid sequence of SEQ ID NO:51 and a VL comprising the amino acid sequence of SEQ ID NO:131.

In some such embodiments, the antibody or antigen binding fragment thereof comprises a VH comprising the amino acid sequence of SEQ ID NO:52 and a VL comprising the amino acid sequence of SEQ ID NO:131.

In some such embodiments, the antibody or antigen binding fragment thereof comprises a VH comprising the amino acid sequence of SEQ ID NO:53 and a VL comprising the amino acid sequence of SEQ ID NO:131.

In some such embodiments, the antibody or antigen binding fragment thereof comprises a VH comprising the amino acid sequence of SEQ ID NO:54 and a VL comprising the amino acid sequence of SEQ ID NO:131.

In some such embodiments, the antibody or antigen binding fragment thereof comprises a VH comprising the amino acid sequence of SEQ ID NO:55 and a VL comprising the amino acid sequence of SEQ ID NO:131.

In some such embodiments, the antibody or antigen binding fragment thereof comprises a VH comprising the amino acid sequence of SEQ ID NO:56 and a VL comprising the amino acid sequence of SEQ ID NO:131.

In some such embodiments, the antibody or antigen binding fragment thereof comprises a VH comprising the amino acid sequence of SEQ ID NO:57 and a VL comprising the amino acid sequence of SEQ ID NO:131.

In some such embodiments, the antibody or antigen binding fragment thereof comprises a VH comprising the amino acid sequence of SEQ ID NO:58 and a VL comprising the amino acid sequence of SEQ ID NO:131.

In some such embodiments, the antibody or antigen binding fragment thereof comprises a VH comprising the amino acid sequence of SEQ ID NO:59 and a VL comprising the amino acid sequence of SEQ ID NO:131.

In some such embodiments, the antibody or antigen binding fragment thereof comprises a VH comprising the amino acid sequence of SEQ ID NO:60 and a VL comprising the amino acid sequence of SEQ ID NO:131.

In some such embodiments, the antibody or antigen binding fragment thereof comprises a VH comprising the amino acid sequence of SEQ ID NO:61 and a VL comprising the amino acid sequence of SEQ ID NO:131.

In some such embodiments, the antibody or antigen binding fragment thereof comprises a VH comprising the amino acid sequence of SEQ ID NO:62 and a VL comprising the amino acid sequence of SEQ ID NO:131.

In some such embodiments, the antibody or antigen binding fragment thereof comprises a VH comprising the amino acid sequence of SEQ ID NO:63 and a VL comprising the amino acid sequence of SEQ ID NO:131.

In some such embodiments, the antibody or antigen binding fragment thereof comprises a VH comprising the amino acid sequence of SEQ ID NO:64 and a VL comprising the amino acid sequence of SEQ ID NO:131.

In some such embodiments, the antibody or antigen binding fragment thereof comprises a VH comprising the amino acid sequence of SEQ ID NO:65 and a VL comprising the amino acid sequence of SEQ ID NO:131.

In some such embodiments, the antibody or antigen binding fragment thereof comprises a VH comprising the amino acid sequence of SEQ ID NO:66 and a VL comprising the amino acid sequence of SEQ ID NO:131.

In some such embodiments, the antibody or antigen binding fragment thereof comprises a VH comprising the amino acid sequence of SEQ ID NO:67 and a VL comprising the amino acid sequence of SEQ ID NO:131.

In some such embodiments, the antibody or antigen binding fragment thereof comprises a VH comprising the amino acid sequence of SEQ ID NO:68 and a VL comprising the amino acid sequence of SEQ ID NO:131.

In some such embodiments, the antibody or antigen binding fragment thereof comprises a VH comprising the amino acid sequence of SEQ ID NO:69 and a VL comprising the amino acid sequence of SEQ ID NO:131.

In some such embodiments, the antibody or antigen binding fragment thereof comprises a VH comprising the amino acid sequence of SEQ ID NO:70 and a VL comprising the amino acid sequence of SEQ ID NO:131.

In some such embodiments, the antibody or antigen binding fragment thereof comprises a VH comprising the amino acid sequence of SEQ ID NO:71 and a VL comprising the amino acid sequence of SEQ ID NO:131.

In some such embodiments, the antibody or antigen binding fragment thereof comprises a VH comprising the amino acid sequence of SEQ ID NO:72 and a VL comprising the amino acid sequence of SEQ ID NO:131.

In some such embodiments, the antibody or antigen binding fragment thereof comprises a VH comprising the amino acid sequence of SEQ ID NO:73 and a VL comprising the amino acid sequence of SEQ ID NO:131.

In some such embodiments, the antibody or antigen binding fragment thereof comprises a VH comprising the amino acid sequence of SEQ ID NO:74 and a VL comprising the amino acid sequence of SEQ ID NO:131.

In some such embodiments, the antibody or antigen binding fragment thereof comprises a VH comprising the amino acid sequence of SEQ ID NO:75 and a VL comprising the amino acid sequence of SEQ ID NO:131.

In some such embodiments, the antibody or antigen binding fragment thereof comprises a VH comprising the amino acid sequence of SEQ ID NO:76 and a VL comprising the amino acid sequence of SEQ ID NO:131.

In some such embodiments, the antibody or antigen binding fragment thereof comprises a VH comprising the amino acid sequence of SEQ ID NO:77 and a VL comprising the amino acid sequence of SEQ ID NO:131.

In some such embodiments, the antibody or antigen binding fragment thereof comprises a VH comprising the amino acid sequence of SEQ ID NO:78 and a VL comprising the amino acid sequence of SEQ ID NO:131.

In some such embodiments, the antibody or antigen binding fragment thereof comprises a VH comprising the amino acid sequence of SEQ ID NO:79 and a VL comprising the amino acid sequence of SEQ ID NO:131.

In some such embodiments, the antibody or antigen binding fragment thereof comprises a VH comprising the amino acid sequence of SEQ ID NO:80 and a VL comprising the amino acid sequence of SEQ ID NO:131.

In some such embodiments, the antibody or antigen binding fragment thereof comprises a VH comprising the amino acid sequence of SEQ ID NO:81 and a VL comprising the amino acid sequence of SEQ ID NO:131.

In some such embodiments, the antibody or antigen binding fragment thereof comprises a VH comprising the amino acid sequence of SEQ ID NO:82 and a VL comprising the amino acid sequence of SEQ ID NO:131.

In some such embodiments, the antibody or antigen binding fragment thereof comprises a VH comprising the amino acid sequence of SEQ ID NO:83 and a VL comprising the amino acid sequence of SEQ ID NO:131.

In some such embodiments, the antibody or antigen binding fragment thereof comprises a VH comprising the amino acid sequence of SEQ ID NO:84 and a VL comprising the amino acid sequence of SEQ ID NO:131.

In some such embodiments, the antibody or antigen binding fragment thereof comprises a VH comprising the amino acid sequence of SEQ ID NO:85 and a VL comprising the amino acid sequence of SEQ ID NO:131.

In some such embodiments, the antibody or antigen binding fragment thereof comprises a VH comprising the amino acid sequence of SEQ ID NO:86 and a VL comprising the amino acid sequence of SEQ ID NO:131.

In some such embodiments, the antibody or antigen binding fragment thereof comprises a VH comprising the amino acid sequence of SEQ ID NO:87 and a VL comprising the amino acid sequence of SEQ ID NO:131.

In some such embodiments, the antibody or antigen binding fragment thereof comprises a VH comprising the amino acid sequence of SEQ ID NO:88 and a VL comprising the amino acid sequence of SEQ ID NO:131.

In some such embodiments, the antibody or antigen binding fragment thereof comprises a VH comprising the amino acid sequence of SEQ ID NO:238 and a VL comprising the amino acid sequence of SEQ ID NO:131.

In some such embodiments, the antibody or antigen binding fragment thereof comprises a VH comprising the amino acid sequence of SEQ ID NO:239 and a VL comprising the amino acid sequence of SEQ ID NO:131.

In some such embodiments, the antibody or antigen binding fragment thereof comprises a VH comprising the amino acid sequence of SEQ ID NO:240 and a VL comprising the amino acid sequence of SEQ ID NO:131.

In some such embodiments, the antibody or antigen binding fragment thereof comprises a VH comprising the amino acid sequence of SEQ ID NO:241 and a VL comprising the amino acid sequence of SEQ ID NO:131.

In some such embodiments, the antibody or antigen binding fragment thereof comprises a VH comprising the amino acid sequence of SEQ ID NO:242 and a VL comprising the amino acid sequence of SEQ ID NO:131.

In some such embodiments, the antibody or antigen binding fragment thereof comprises a VH comprising the amino acid sequence of SEQ ID NO:243 and a VL comprising the amino acid sequence of SEQ ID NO:131.

In some such embodiments, the antibody or antigen binding fragment thereof comprises a VH comprising the amino acid sequence of SEQ ID NO:43 and a VL comprising the amino acid sequence of SEQ ID NO:132.

In some such embodiments, the antibody or antigen binding fragment thereof comprises a VH comprising the amino acid sequence of SEQ ID NO:44 and a VL comprising the amino acid sequence of SEQ ID NO:132.

In some such embodiments, the antibody or antigen binding fragment thereof comprises a VH comprising the amino acid sequence of SEQ ID NO:45 and a VL comprising the amino acid sequence of SEQ ID NO:132.

In some such embodiments, the antibody or antigen binding fragment thereof comprises a VH comprising the amino acid sequence of SEQ ID NO:46 and a VL comprising the amino acid sequence of SEQ ID NO:132.

In some such embodiments, the antibody or antigen binding fragment thereof comprises a VH comprising the amino acid sequence of SEQ ID NO:47 and a VL comprising the amino acid sequence of SEQ ID NO:132.

In some such embodiments, the antibody or antigen binding fragment thereof comprises a VH comprising the amino acid sequence of SEQ ID NO:48 and a VL comprising the amino acid sequence of SEQ ID NO:132.

In some such embodiments, the antibody or antigen binding fragment thereof comprises a VH comprising the amino acid sequence of SEQ ID NO:49 and a VL comprising the amino acid sequence of SEQ ID NO:132.

In some such embodiments, the antibody or antigen binding fragment thereof comprises a VH comprising the amino acid sequence of SEQ ID NO:50 and a VL comprising the amino acid sequence of SEQ ID NO:132.

In some such embodiments, the antibody or antigen binding fragment thereof comprises a VH comprising the amino acid sequence of SEQ ID NO:51 and a VL comprising the amino acid sequence of SEQ ID NO:132.

In some such embodiments, the antibody or antigen binding fragment thereof comprises a VH comprising the amino acid sequence of SEQ ID NO:52 and a VL comprising the amino acid sequence of SEQ ID NO:132.

In some such embodiments, the antibody or antigen binding fragment thereof comprises a VH comprising the amino acid sequence of SEQ ID NO:53 and a VL comprising the amino acid sequence of SEQ ID NO:132.

In some such embodiments, the antibody or antigen binding fragment thereof comprises a VH comprising the amino acid sequence of SEQ ID NO:54 and a VL comprising the amino acid sequence of SEQ ID NO:132.

In some such embodiments, the antibody or antigen binding fragment thereof comprises a VH comprising the amino acid sequence of SEQ ID NO:55 and a VL comprising the amino acid sequence of SEQ ID NO:132.

In some such embodiments, the antibody or antigen binding fragment thereof comprises a VH comprising the amino acid sequence of SEQ ID NO:56 and a VL comprising the amino acid sequence of SEQ ID NO:132.

In some such embodiments, the antibody or antigen binding fragment thereof comprises a VH comprising the amino acid sequence of SEQ ID NO:57 and a VL comprising the amino acid sequence of SEQ ID NO:132.

In some such embodiments, the antibody or antigen binding fragment thereof comprises a VH comprising the amino acid sequence of SEQ ID NO:58 and a VL comprising the amino acid sequence of SEQ ID NO:132.

In some such embodiments, the antibody or antigen binding fragment thereof comprises a VH comprising the amino acid sequence of SEQ ID NO:59 and a VL comprising the amino acid sequence of SEQ ID NO:132.

In some such embodiments, the antibody or antigen binding fragment thereof comprises a VH comprising the amino acid sequence of SEQ ID NO:60 and a VL comprising the amino acid sequence of SEQ ID NO:132.

In some such embodiments, the antibody or antigen binding fragment thereof comprises a VH comprising the amino acid sequence of SEQ ID NO:61 and a VL comprising the amino acid sequence of SEQ ID NO:132.

In some such embodiments, the antibody or antigen binding fragment thereof comprises a VH comprising the amino acid sequence of SEQ ID NO:62 and a VL comprising the amino acid sequence of SEQ ID NO:132.

In some such embodiments, the antibody or antigen binding fragment thereof comprises a VH comprising the amino acid sequence of SEQ ID NO:63 and a VL comprising the amino acid sequence of SEQ ID NO:132.

In some such embodiments, the antibody or antigen binding fragment thereof comprises a VH comprising the amino acid sequence of SEQ ID NO:64 and a VL comprising the amino acid sequence of SEQ ID NO:132.

In some such embodiments, the antibody or antigen binding fragment thereof comprises a VH comprising the amino acid sequence of SEQ ID NO:65 and a VL comprising the amino acid sequence of SEQ ID NO:132.

In some such embodiments, the antibody or antigen binding fragment thereof comprises a VH comprising the amino acid sequence of SEQ ID NO:66 and a VL comprising the amino acid sequence of SEQ ID NO:132.

In some such embodiments, the antibody or antigen binding fragment thereof comprises a VH comprising the amino acid sequence of SEQ ID NO:67 and a VL comprising the amino acid sequence of SEQ ID NO:132.

In some such embodiments, the antibody or antigen binding fragment thereof comprises a VH comprising the amino acid sequence of SEQ ID NO:68 and a VL comprising the amino acid sequence of SEQ ID NO:132.

In some such embodiments, the antibody or antigen binding fragment thereof comprises a VH comprising the amino acid sequence of SEQ ID NO:69 and a VL comprising the amino acid sequence of SEQ ID NO:132.

In some such embodiments, the antibody or antigen binding fragment thereof comprises a VH comprising the amino acid sequence of SEQ ID NO:70 and a VL comprising the amino acid sequence of SEQ ID NO:132.

In some such embodiments, the antibody or antigen binding fragment thereof comprises a VH comprising the amino acid sequence of SEQ ID NO:71 and a VL comprising the amino acid sequence of SEQ ID NO:132.

In some such embodiments, the antibody or antigen binding fragment thereof comprises a VH comprising the amino acid sequence of SEQ ID NO:72 and a VL comprising the amino acid sequence of SEQ ID NO:132.

In some such embodiments, the antibody or antigen binding fragment thereof comprises a VH comprising the amino acid sequence of SEQ ID NO:73 and a VL comprising the amino acid sequence of SEQ ID NO:132.

In some such embodiments, the antibody or antigen binding fragment thereof comprises a VH comprising the amino acid sequence of SEQ ID NO:74 and a VL comprising the amino acid sequence of SEQ ID NO:132.

In some such embodiments, the antibody or antigen binding fragment thereof comprises a VH comprising the amino acid sequence of SEQ ID NO:75 and a VL comprising the amino acid sequence of SEQ ID NO:132.

In some such embodiments, the antibody or antigen binding fragment thereof comprises a VH comprising the amino acid sequence of SEQ ID NO:76 and a VL comprising the amino acid sequence of SEQ ID NO:132.

In some such embodiments, the antibody or antigen binding fragment thereof comprises a VH comprising the amino acid sequence of SEQ ID NO:77 and a VL comprising the amino acid sequence of SEQ ID NO:132.

In some such embodiments, the antibody or antigen binding fragment thereof comprises a VH comprising the amino acid sequence of SEQ ID NO:78 and a VL comprising the amino acid sequence of SEQ ID NO:132.

In some such embodiments, the antibody or antigen binding fragment thereof comprises a VH comprising the amino acid sequence of SEQ ID NO:79 and a VL comprising the amino acid sequence of SEQ ID NO:132.

In some such embodiments, the antibody or antigen binding fragment thereof comprises a VH comprising the amino acid sequence of SEQ ID NO:80 and a VL comprising the amino acid sequence of SEQ ID NO:132.

In some such embodiments, the antibody or antigen binding fragment thereof comprises a VH comprising the amino acid sequence of SEQ ID NO:81 and a VL comprising the amino acid sequence of SEQ ID NO:132.

In some such embodiments, the antibody or antigen binding fragment thereof comprises a VH comprising the amino acid sequence of SEQ ID NO:82 and a VL comprising the amino acid sequence of SEQ ID NO:132.

In some such embodiments, the antibody or antigen binding fragment thereof comprises a VH comprising the amino acid sequence of SEQ ID NO:83 and a VL comprising the amino acid sequence of SEQ ID NO:132.

In some such embodiments, the antibody or antigen binding fragment thereof comprises a VH comprising the amino acid sequence of SEQ ID NO:84 and a VL comprising the amino acid sequence of SEQ ID NO:132.

In some such embodiments, the antibody or antigen binding fragment thereof comprises a VH comprising the amino acid sequence of SEQ ID NO:85 and a VL comprising the amino acid sequence of SEQ ID NO:132.

In some such embodiments, the antibody or antigen binding fragment thereof comprises a VH comprising the amino acid sequence of SEQ ID NO:86 and a VL comprising the amino acid sequence of SEQ ID NO:132.

In some such embodiments, the antibody or antigen binding fragment thereof comprises a VH comprising the amino acid sequence of SEQ ID NO:87 and a VL comprising the amino acid sequence of SEQ ID NO:132.

In some such embodiments, the antibody or antigen binding fragment thereof comprises a VH comprising the amino acid sequence of SEQ ID NO:88 and a VL comprising the amino acid sequence of SEQ ID NO:132.

In some such embodiments, the antibody or antigen binding fragment thereof comprises a VH comprising the amino acid sequence of SEQ ID NO:238 and a VL comprising the amino acid sequence of SEQ ID NO:132.

In some such embodiments, the antibody or antigen binding fragment thereof comprises a VH comprising the amino acid sequence of SEQ ID NO:239 and a VL comprising the amino acid sequence of SEQ ID NO:132.

In some such embodiments, the antibody or antigen binding fragment thereof comprises a VH comprising the amino acid sequence of SEQ ID NO:240 and a VL comprising the amino acid sequence of SEQ ID NO:132.

In some such embodiments, the antibody or antigen binding fragment thereof comprises a VH comprising the amino acid sequence of SEQ ID NO:241 and a VL comprising the amino acid sequence of SEQ ID NO:132.

In some such embodiments, the antibody or antigen binding fragment thereof comprises a VH comprising the amino acid sequence of SEQ ID NO:242 and a VL comprising the amino acid sequence of SEQ ID NO:132.

In some such embodiments, the antibody or antigen binding fragment thereof comprises a VH comprising the amino acid sequence of SEQ ID NO:243 and a VL comprising the amino acid sequence of SEQ ID NO:132.

In some such embodiments, the antibody or antigen binding fragment thereof comprises a VH comprising the amino acid sequence of SEQ ID NO:43 and a VL comprising the amino acid sequence of SEQ ID NO:133.

In some such embodiments, the antibody or antigen binding fragment thereof comprises a VH comprising the amino acid sequence of SEQ ID NO:44 and a VL comprising the amino acid sequence of SEQ ID NO:133.

In some such embodiments, the antibody or antigen binding fragment thereof comprises a VH comprising the amino acid sequence of SEQ ID NO:45 and a VL comprising the amino acid sequence of SEQ ID NO:133.

In some such embodiments, the antibody or antigen binding fragment thereof comprises a VH comprising the amino acid sequence of SEQ ID NO:46 and a VL comprising the amino acid sequence of SEQ ID NO:133.

In some such embodiments, the antibody or antigen binding fragment thereof comprises a VH comprising the amino acid sequence of SEQ ID NO:47 and a VL comprising the amino acid sequence of SEQ ID NO:133.

In some such embodiments, the antibody or antigen binding fragment thereof comprises a VH comprising the amino acid sequence of SEQ ID NO:48 and a VL comprising the amino acid sequence of SEQ ID NO:133.

In some such embodiments, the antibody or antigen binding fragment thereof comprises a VH comprising the amino acid sequence of SEQ ID NO:49 and a VL comprising the amino acid sequence of SEQ ID NO:133.

In some such embodiments, the antibody or antigen binding fragment thereof comprises a VH comprising the amino acid sequence of SEQ ID NO:50 and a VL comprising the amino acid sequence of SEQ ID NO:133.

In some such embodiments, the antibody or antigen binding fragment thereof comprises a VH comprising the amino acid sequence of SEQ ID NO:51 and a VL comprising the amino acid sequence of SEQ ID NO:133.

In some such embodiments, the antibody or antigen binding fragment thereof comprises a VH comprising the amino acid sequence of SEQ ID NO:52 and a VL comprising the amino acid sequence of SEQ ID NO:133.

In some such embodiments, the antibody or antigen binding fragment thereof comprises a VH comprising the amino acid sequence of SEQ ID NO:53 and a VL comprising the amino acid sequence of SEQ ID NO:133.

In some such embodiments, the antibody or antigen binding fragment thereof comprises a VH comprising the amino acid sequence of SEQ ID NO:54 and a VL comprising the amino acid sequence of SEQ ID NO:133.

In some such embodiments, the antibody or antigen binding fragment thereof comprises a VH comprising the amino acid sequence of SEQ ID NO:55 and a VL comprising the amino acid sequence of SEQ ID NO:133.

In some such embodiments, the antibody or antigen binding fragment thereof comprises a VH comprising the amino acid sequence of SEQ ID NO:56 and a VL comprising the amino acid sequence of SEQ ID NO:133.

In some such embodiments, the antibody or antigen binding fragment thereof comprises a VH comprising the amino acid sequence of SEQ ID NO:57 and a VL comprising the amino acid sequence of SEQ ID NO:133.

In some such embodiments, the antibody or antigen binding fragment thereof comprises a VH comprising the amino acid sequence of SEQ ID NO:58 and a VL comprising the amino acid sequence of SEQ ID NO:133.

In some such embodiments, the antibody or antigen binding fragment thereof comprises a VH comprising the amino acid sequence of SEQ ID NO:59 and a VL comprising the amino acid sequence of SEQ ID NO:133.

In some such embodiments, the antibody or antigen binding fragment thereof comprises a VH comprising the amino acid sequence of SEQ ID NO:60 and a VL comprising the amino acid sequence of SEQ ID NO:133.

In some such embodiments, the antibody or antigen binding fragment thereof comprises a VH comprising the amino acid sequence of SEQ ID NO:61 and a VL comprising the amino acid sequence of SEQ ID NO:133.

In some such embodiments, the antibody or antigen binding fragment thereof comprises a VH comprising the amino acid sequence of SEQ ID NO:62 and a VL comprising the amino acid sequence of SEQ ID NO:133.

In some such embodiments, the antibody or antigen binding fragment thereof comprises a VH comprising the amino acid sequence of SEQ ID NO:63 and a VL comprising the amino acid sequence of SEQ ID NO:133.

In some such embodiments, the antibody or antigen binding fragment thereof comprises a VH comprising the amino acid sequence of SEQ ID NO:64 and a VL comprising the amino acid sequence of SEQ ID NO:133.

In some such embodiments, the antibody or antigen binding fragment thereof comprises a VH comprising the amino acid sequence of SEQ ID NO:65 and a VL comprising the amino acid sequence of SEQ ID NO:133.

In some such embodiments, the antibody or antigen binding fragment thereof comprises a VH comprising the amino acid sequence of SEQ ID NO:66 and a VL comprising the amino acid sequence of SEQ ID NO:133.

In some such embodiments, the antibody or antigen binding fragment thereof comprises a VH comprising the amino acid sequence of SEQ ID NO:67 and a VL comprising the amino acid sequence of SEQ ID NO:133.

In some such embodiments, the antibody or antigen binding fragment thereof comprises a VH comprising the amino acid sequence of SEQ ID NO:68 and a VL comprising the amino acid sequence of SEQ ID NO:133.

In some such embodiments, the antibody or antigen binding fragment thereof comprises a VH comprising the amino acid sequence of SEQ ID NO:69 and a VL comprising the amino acid sequence of SEQ ID NO:133.

In some such embodiments, the antibody or antigen binding fragment thereof comprises a VH comprising the amino acid sequence of SEQ ID NO:70 and a VL comprising the amino acid sequence of SEQ ID NO:133.

In some such embodiments, the antibody or antigen binding fragment thereof comprises a VH comprising the amino acid sequence of SEQ ID NO:71 and a VL comprising the amino acid sequence of SEQ ID NO:133.

In some such embodiments, the antibody or antigen binding fragment thereof comprises a VH comprising the amino acid sequence of SEQ ID NO:72 and a VL comprising the amino acid sequence of SEQ ID NO:133.

In some such embodiments, the antibody or antigen binding fragment thereof comprises a VH comprising the amino acid sequence of SEQ ID NO:73 and a VL comprising the amino acid sequence of SEQ ID NO:133.

In some such embodiments, the antibody or antigen binding fragment thereof comprises a VH comprising the amino acid sequence of SEQ ID NO:74 and a VL comprising the amino acid sequence of SEQ ID NO:133.

In some such embodiments, the antibody or antigen binding fragment thereof comprises a VH comprising the amino acid sequence of SEQ ID NO:75 and a VL comprising the amino acid sequence of SEQ ID NO:133.

In some such embodiments, the antibody or antigen binding fragment thereof comprises a VH comprising the amino acid sequence of SEQ ID NO:76 and a VL comprising the amino acid sequence of SEQ ID NO:133.

In some such embodiments, the antibody or antigen binding fragment thereof comprises a VH comprising the amino acid sequence of SEQ ID NO:77 and a VL comprising the amino acid sequence of SEQ ID NO:133.

In some such embodiments, the antibody or antigen binding fragment thereof comprises a VH comprising the amino acid sequence of SEQ ID NO:78 and a VL comprising the amino acid sequence of SEQ ID NO:133.

In some such embodiments, the antibody or antigen binding fragment thereof comprises a VH comprising the amino acid sequence of SEQ ID NO:79 and a VL comprising the amino acid sequence of SEQ ID NO:133.

In some such embodiments, the antibody or antigen binding fragment thereof comprises a VH comprising the amino acid sequence of SEQ ID NO:80 and a VL comprising the amino acid sequence of SEQ ID NO:133.

In some such embodiments, the antibody or antigen binding fragment thereof comprises a VH comprising the amino acid sequence of SEQ ID NO:81 and a VL comprising the amino acid sequence of SEQ ID NO:133.

In some such embodiments, the antibody or antigen binding fragment thereof comprises a VH comprising the amino acid sequence of SEQ ID NO:82 and a VL comprising the amino acid sequence of SEQ ID NO:133.

In some such embodiments, the antibody or antigen binding fragment thereof comprises a VH comprising the amino acid sequence of SEQ ID NO:83 and a VL comprising the amino acid sequence of SEQ ID NO:133.

In some such embodiments, the antibody or antigen binding fragment thereof comprises a VH comprising the amino acid sequence of SEQ ID NO:84 and a VL comprising the amino acid sequence of SEQ ID NO:133.

In some such embodiments, the antibody or antigen binding fragment thereof comprises a VH comprising the amino acid sequence of SEQ ID NO:85 and a VL comprising the amino acid sequence of SEQ ID NO:133.

In some such embodiments, the antibody or antigen binding fragment thereof comprises a VH comprising the amino acid sequence of SEQ ID NO:86 and a VL comprising the amino acid sequence of SEQ ID NO:133.

In some such embodiments, the antibody or antigen binding fragment thereof comprises a VH comprising the amino acid sequence of SEQ ID NO:87 and a VL comprising the amino acid sequence of SEQ ID NO:133.

In some such embodiments, the antibody or antigen binding fragment thereof comprises a VH comprising the amino acid sequence of SEQ ID NO:88 and a VL comprising the amino acid sequence of SEQ ID NO:133.

In some such embodiments, the antibody or antigen binding fragment thereof comprises a VH comprising the amino acid sequence of SEQ ID NO:238 and a VL comprising the amino acid sequence of SEQ ID NO:133.

In some such embodiments, the antibody or antigen binding fragment thereof comprises a VH comprising the amino acid sequence of SEQ ID NO:239 and a VL comprising the amino acid sequence of SEQ ID NO:133.

In some such embodiments, the antibody or antigen binding fragment thereof comprises a VH comprising the amino acid sequence of SEQ ID NO:240 and a VL comprising the amino acid sequence of SEQ ID NO:133.

In some such embodiments, the antibody or antigen binding fragment thereof comprises a VH comprising the amino acid sequence of SEQ ID NO:241 and a VL comprising the amino acid sequence of SEQ ID NO:133.

In some such embodiments, the antibody or antigen binding fragment thereof comprises a VH comprising the amino acid sequence of SEQ ID NO:242 and a VL comprising the amino acid sequence of SEQ ID NO:133.

In some such embodiments, the antibody or antigen binding fragment thereof comprises a VH comprising the amino acid sequence of SEQ ID NO:243 and a VL comprising the amino acid sequence of SEQ ID NO:133.

In some such embodiments, the antibody or antigen binding fragment thereof comprises a VH comprising the amino acid sequence of SEQ ID NO:43 and a VL comprising the amino acid sequence of SEQ ID NO:244.

In some such embodiments, the antibody or antigen binding fragment thereof comprises a VH comprising the amino acid sequence of SEQ ID NO:44 and a VL comprising the amino acid sequence of SEQ ID NO:244.

In some such embodiments, the antibody or antigen binding fragment thereof comprises a VH comprising the amino acid sequence of SEQ ID NO:45 and a VL comprising the amino acid sequence of SEQ ID NO:244.

In some such embodiments, the antibody or antigen binding fragment thereof comprises a VH comprising the amino acid sequence of SEQ ID NO:46 and a VL comprising the amino acid sequence of SEQ ID NO:244.

In some such embodiments, the antibody or antigen binding fragment thereof comprises a VH comprising the amino acid sequence of SEQ ID NO:47 and a VL comprising the amino acid sequence of SEQ ID NO:244.

In some such embodiments, the antibody or antigen binding fragment thereof comprises a VH comprising the amino acid sequence of SEQ ID NO:48 and a VL comprising the amino acid sequence of SEQ ID NO:244.

In some such embodiments, the antibody or antigen binding fragment thereof comprises a VH comprising the amino acid sequence of SEQ ID NO:49 and a VL comprising the amino acid sequence of SEQ ID NO:244.

In some such embodiments, the antibody or antigen binding fragment thereof comprises a VH comprising the amino acid sequence of SEQ ID NO:50 and a VL comprising the amino acid sequence of SEQ ID NO:244.

In some such embodiments, the antibody or antigen binding fragment thereof comprises a VH comprising the amino acid sequence of SEQ ID NO:51 and a VL comprising the amino acid sequence of SEQ ID NO:244.

In some such embodiments, the antibody or antigen binding fragment thereof comprises a VH comprising the amino acid sequence of SEQ ID NO:52 and a VL comprising the amino acid sequence of SEQ ID NO:244.

In some such embodiments, the antibody or antigen binding fragment thereof comprises a VH comprising the amino acid sequence of SEQ ID NO:53 and a VL comprising the amino acid sequence of SEQ ID NO:244.

In some such embodiments, the antibody or antigen binding fragment thereof comprises a VH comprising the amino acid sequence of SEQ ID NO:54 and a VL comprising the amino acid sequence of SEQ ID NO:244.

In some such embodiments, the antibody or antigen binding fragment thereof comprises a VH comprising the amino acid sequence of SEQ ID NO:55 and a VL comprising the amino acid sequence of SEQ ID NO:244.

In some such embodiments, the antibody or antigen binding fragment thereof comprises a VH comprising the amino acid sequence of SEQ ID NO:56 and a VL comprising the amino acid sequence of SEQ ID NO:244.

In some such embodiments, the antibody or antigen binding fragment thereof comprises a VH comprising the amino acid sequence of SEQ ID NO:57 and a VL comprising the amino acid sequence of SEQ ID NO:244.

In some such embodiments, the antibody or antigen binding fragment thereof comprises a VH comprising the amino acid sequence of SEQ ID NO:58 and a VL comprising the amino acid sequence of SEQ ID NO:244.

In some such embodiments, the antibody or antigen binding fragment thereof comprises a VH comprising the amino acid sequence of SEQ ID NO:59 and a VL comprising the amino acid sequence of SEQ ID NO:244.

In some such embodiments, the antibody or antigen binding fragment thereof comprises a VH comprising the amino acid sequence of SEQ ID NO:60 and a VL comprising the amino acid sequence of SEQ ID NO:244.

In some such embodiments, the antibody or antigen binding fragment thereof comprises a VH comprising the amino acid sequence of SEQ ID NO:61 and a VL comprising the amino acid sequence of SEQ ID NO:244.

In some such embodiments, the antibody or antigen binding fragment thereof comprises a VH comprising the amino acid sequence of SEQ ID NO:62 and a VL comprising the amino acid sequence of SEQ ID NO:244.

In some such embodiments, the antibody or antigen binding fragment thereof comprises a VH comprising the amino acid sequence of SEQ ID NO:63 and a VL comprising the amino acid sequence of SEQ ID NO:244.

In some such embodiments, the antibody or antigen binding fragment thereof comprises a VH comprising the amino acid sequence of SEQ ID NO:64 and a VL comprising the amino acid sequence of SEQ ID NO:244.

In some such embodiments, the antibody or antigen binding fragment thereof comprises a VH comprising the amino acid sequence of SEQ ID NO:65 and a VL comprising the amino acid sequence of SEQ ID NO:244.

In some such embodiments, the antibody or antigen binding fragment thereof comprises a VH comprising the amino acid sequence of SEQ ID NO:66 and a VL comprising the amino acid sequence of SEQ ID NO:244.

In some such embodiments, the antibody or antigen binding fragment thereof comprises a VH comprising the amino acid sequence of SEQ ID NO:67 and a VL comprising the amino acid sequence of SEQ ID NO:244.

In some such embodiments, the antibody or antigen binding fragment thereof comprises a VH comprising the amino acid sequence of SEQ ID NO:68 and a VL comprising the amino acid sequence of SEQ ID NO:244.

In some such embodiments, the antibody or antigen binding fragment thereof comprises a VH comprising the amino acid sequence of SEQ ID NO:69 and a VL comprising the amino acid sequence of SEQ ID NO:244.

In some such embodiments, the antibody or antigen binding fragment thereof comprises a VH comprising the amino acid sequence of SEQ ID NO:70 and a VL comprising the amino acid sequence of SEQ ID NO:244.

In some such embodiments, the antibody or antigen binding fragment thereof comprises a VH comprising the amino acid sequence of SEQ ID NO:71 and a VL comprising the amino acid sequence of SEQ ID NO:244.

In some such embodiments, the antibody or antigen binding fragment thereof comprises a VH comprising the amino acid sequence of SEQ ID NO:72 and a VL comprising the amino acid sequence of SEQ ID NO:244.

In some such embodiments, the antibody or antigen binding fragment thereof comprises a VH comprising the amino acid sequence of SEQ ID NO:73 and a VL comprising the amino acid sequence of SEQ ID NO:244.

In some such embodiments, the antibody or antigen binding fragment thereof comprises a VH comprising the amino acid sequence of SEQ ID NO:74 and a VL comprising the amino acid sequence of SEQ ID NO:244.

In some such embodiments, the antibody or antigen binding fragment thereof comprises a VH comprising the amino acid sequence of SEQ ID NO:75 and a VL comprising the amino acid sequence of SEQ ID NO:244.

In some such embodiments, the antibody or antigen binding fragment thereof comprises a VH comprising the amino acid sequence of SEQ ID NO:76 and a VL comprising the amino acid sequence of SEQ ID NO:244.

In some such embodiments, the antibody or antigen binding fragment thereof comprises a VH comprising the amino acid sequence of SEQ ID NO:77 and a VL comprising the amino acid sequence of SEQ ID NO:244.

In some such embodiments, the antibody or antigen binding fragment thereof comprises a VH comprising the amino acid sequence of SEQ ID NO:78 and a VL comprising the amino acid sequence of SEQ ID NO:244.

In some such embodiments, the antibody or antigen binding fragment thereof comprises a VH comprising the amino acid sequence of SEQ ID NO:79 and a VL comprising the amino acid sequence of SEQ ID NO:244.

In some such embodiments, the antibody or antigen binding fragment thereof comprises a VH comprising the amino acid sequence of SEQ ID NO:80 and a VL comprising the amino acid sequence of SEQ ID NO:244.

In some such embodiments, the antibody or antigen binding fragment thereof comprises a VH comprising the amino acid sequence of SEQ ID NO:81 and a VL comprising the amino acid sequence of SEQ ID NO:244.

In some such embodiments, the antibody or antigen binding fragment thereof comprises a VH comprising the amino acid sequence of SEQ ID NO:82 and a VL comprising the amino acid sequence of SEQ ID NO:244.

In some such embodiments, the antibody or antigen binding fragment thereof comprises a VH comprising the amino acid sequence of SEQ ID NO:83 and a VL comprising the amino acid sequence of SEQ ID NO:244.

In some such embodiments, the antibody or antigen binding fragment thereof comprises a VH comprising the amino acid sequence of SEQ ID NO:84 and a VL comprising the amino acid sequence of SEQ ID NO:244.

In some such embodiments, the antibody or antigen binding fragment thereof comprises a VH comprising the amino acid sequence of SEQ ID NO:85 and a VL comprising the amino acid sequence of SEQ ID NO:244.

In some such embodiments, the antibody or antigen binding fragment thereof comprises a VH comprising the amino acid sequence of SEQ ID NO:86 and a VL comprising the amino acid sequence of SEQ ID NO:244.

In some such embodiments, the antibody or antigen binding fragment thereof comprises a VH comprising the amino acid sequence of SEQ ID NO:87 and a VL comprising the amino acid sequence of SEQ ID NO:244.

In some such embodiments, the antibody or antigen binding fragment thereof comprises a VH comprising the amino acid sequence of SEQ ID NO:88 and a VL comprising the amino acid sequence of SEQ ID NO:244.

In some such embodiments, the antibody or antigen binding fragment thereof comprises a VH comprising the amino acid sequence of SEQ ID NO:238 and a VL comprising the amino acid sequence of SEQ ID NO:244.

In some such embodiments, the antibody or antigen binding fragment thereof comprises a VH comprising the amino acid sequence of SEQ ID NO:239 and a VL comprising the amino acid sequence of SEQ ID NO:244.

In some such embodiments, the antibody or antigen binding fragment thereof comprises a VH comprising the amino acid sequence of SEQ ID NO:240 and a VL comprising the amino acid sequence of SEQ ID NO:244.

In some such embodiments, the antibody or antigen binding fragment thereof comprises a VH comprising the amino acid sequence of SEQ ID NO:241 and a VL comprising the amino acid sequence of SEQ ID NO:244.

In some such embodiments, the antibody or antigen binding fragment thereof comprises a VH comprising the amino acid sequence of SEQ ID NO:242 and a VL comprising the amino acid sequence of SEQ ID NO:244.

In some such embodiments, the antibody or antigen binding fragment thereof comprises a VH comprising the amino acid sequence of SEQ ID NO:243 and a VL comprising the amino acid sequence of SEQ ID NO:244.

In some such embodiments, the antibody or antigen binding fragment thereof comprises a VH comprising the amino acid sequence of SEQ ID NO:43 and a VL comprising the amino acid sequence of SEQ ID NO:245.

In some such embodiments, the antibody or antigen binding fragment thereof comprises a VH comprising the amino acid sequence of SEQ ID NO:44 and a VL comprising the amino acid sequence of SEQ ID NO:245.

In some such embodiments, the antibody or antigen binding fragment thereof comprises a VH comprising the amino acid sequence of SEQ ID NO:45 and a VL comprising the amino acid sequence of SEQ ID NO:245.

In some such embodiments, the antibody or antigen binding fragment thereof comprises a VH comprising the amino acid sequence of SEQ ID NO:46 and a VL comprising the amino acid sequence of SEQ ID NO:245.

In some such embodiments, the antibody or antigen binding fragment thereof comprises a VH comprising the amino acid sequence of SEQ ID NO:47 and a VL comprising the amino acid sequence of SEQ ID NO:245.

In some such embodiments, the antibody or antigen binding fragment thereof comprises a VH comprising the amino acid sequence of SEQ ID NO:48 and a VL comprising the amino acid sequence of SEQ ID NO:245.

In some such embodiments, the antibody or antigen binding fragment thereof comprises a VH comprising the amino acid sequence of SEQ ID NO:49 and a VL comprising the amino acid sequence of SEQ ID NO:245.

In some such embodiments, the antibody or antigen binding fragment thereof comprises a VH comprising the amino acid sequence of SEQ ID NO:50 and a VL comprising the amino acid sequence of SEQ ID NO:245.

In some such embodiments, the antibody or antigen binding fragment thereof comprises a VH comprising the amino acid sequence of SEQ ID NO:51 and a VL comprising the amino acid sequence of SEQ ID NO:245.

In some such embodiments, the antibody or antigen binding fragment thereof comprises a VH comprising the amino acid sequence of SEQ ID NO:52 and a VL comprising the amino acid sequence of SEQ ID NO:245.

In some such embodiments, the antibody or antigen binding fragment thereof comprises a VH comprising the amino acid sequence of SEQ ID NO:53 and a VL comprising the amino acid sequence of SEQ ID NO:245.

In some such embodiments, the antibody or antigen binding fragment thereof comprises a VH comprising the amino acid sequence of SEQ ID NO:54 and a VL comprising the amino acid sequence of SEQ ID NO:245.

In some such embodiments, the antibody or antigen binding fragment thereof comprises a VH comprising the amino acid sequence of SEQ ID NO:55 and a VL comprising the amino acid sequence of SEQ ID NO:245.

In some such embodiments, the antibody or antigen binding fragment thereof comprises a VH comprising the amino acid sequence of SEQ ID NO:56 and a VL comprising the amino acid sequence of SEQ ID NO:245.

In some such embodiments, the antibody or antigen binding fragment thereof comprises a VH comprising the amino acid sequence of SEQ ID NO:57 and a VL comprising the amino acid sequence of SEQ ID NO:245.

In some such embodiments, the antibody or antigen binding fragment thereof comprises a VH comprising the amino acid sequence of SEQ ID NO:58 and a VL comprising the amino acid sequence of SEQ ID NO:245.

In some such embodiments, the antibody or antigen binding fragment thereof comprises a VH comprising the amino acid sequence of SEQ ID NO:59 and a VL comprising the amino acid sequence of SEQ ID NO:245.

In some such embodiments, the antibody or antigen binding fragment thereof comprises a VH comprising the amino acid sequence of SEQ ID NO:60 and a VL comprising the amino acid sequence of SEQ ID NO:245.

In some such embodiments, the antibody or antigen binding fragment thereof comprises a VH comprising the amino acid sequence of SEQ ID NO:61 and a VL comprising the amino acid sequence of SEQ ID NO:245.

In some such embodiments, the antibody or antigen binding fragment thereof comprises a VH comprising the amino acid sequence of SEQ ID NO:62 and a VL comprising the amino acid sequence of SEQ ID NO:245.

In some such embodiments, the antibody or antigen binding fragment thereof comprises a VH comprising the amino acid sequence of SEQ ID NO:63 and a VL comprising the amino acid sequence of SEQ ID NO:245.

In some such embodiments, the antibody or antigen binding fragment thereof comprises a VH comprising the amino acid sequence of SEQ ID NO:64 and a VL comprising the amino acid sequence of SEQ ID NO:245.

In some such embodiments, the antibody or antigen binding fragment thereof comprises a VH comprising the amino acid sequence of SEQ ID NO:65 and a VL comprising the amino acid sequence of SEQ ID NO:245.

In some such embodiments, the antibody or antigen binding fragment thereof comprises a VH comprising the amino acid sequence of SEQ ID NO:66 and a VL comprising the amino acid sequence of SEQ ID NO:245.

In some such embodiments, the antibody or antigen binding fragment thereof comprises a VH comprising the amino acid sequence of SEQ ID NO:67 and a VL comprising the amino acid sequence of SEQ ID NO:245.

In some such embodiments, the antibody or antigen binding fragment thereof comprises a VH comprising the amino acid sequence of SEQ ID NO:68 and a VL comprising the amino acid sequence of SEQ ID NO:245.

In some such embodiments, the antibody or antigen binding fragment thereof comprises a VH comprising the amino acid sequence of SEQ ID NO:69 and a VL comprising the amino acid sequence of SEQ ID NO:245.

In some such embodiments, the antibody or antigen binding fragment thereof comprises a VH comprising the amino acid sequence of SEQ ID NO:70 and a VL comprising the amino acid sequence of SEQ ID NO:245.

In some such embodiments, the antibody or antigen binding fragment thereof comprises a VH comprising the amino acid sequence of SEQ ID NO:71 and a VL comprising the amino acid sequence of SEQ ID NO:245.

In some such embodiments, the antibody or antigen binding fragment thereof comprises a VH comprising the amino acid sequence of SEQ ID NO:72 and a VL comprising the amino acid sequence of SEQ ID NO:245.

In some such embodiments, the antibody or antigen binding fragment thereof comprises a VH comprising the amino acid sequence of SEQ ID NO:73 and a VL comprising the amino acid sequence of SEQ ID NO:245.

In some such embodiments, the antibody or antigen binding fragment thereof comprises a VH comprising the amino acid sequence of SEQ ID NO:74 and a VL comprising the amino acid sequence of SEQ ID NO:245.

In some such embodiments, the antibody or antigen binding fragment thereof comprises a VH comprising the amino acid sequence of SEQ ID NO:75 and a VL comprising the amino acid sequence of SEQ ID NO:245.

In some such embodiments, the antibody or antigen binding fragment thereof comprises a VH comprising the amino acid sequence of SEQ ID NO:76 and a VL comprising the amino acid sequence of SEQ ID NO:245.

In some such embodiments, the antibody or antigen binding fragment thereof comprises a VH comprising the amino acid sequence of SEQ ID NO:77 and a VL comprising the amino acid sequence of SEQ ID NO:245.

In some such embodiments, the antibody or antigen binding fragment thereof comprises a VH comprising the amino acid sequence of SEQ ID NO:78 and a VL comprising the amino acid sequence of SEQ ID NO:245.

In some such embodiments, the antibody or antigen binding fragment thereof comprises a VH comprising the amino acid sequence of SEQ ID NO:79 and a VL comprising the amino acid sequence of SEQ ID NO:245.

In some such embodiments, the antibody or antigen binding fragment thereof comprises a VH comprising the amino acid sequence of SEQ ID NO:80 and a VL comprising the amino acid sequence of SEQ ID NO:245.

In some such embodiments, the antibody or antigen binding fragment thereof comprises a VH comprising the amino acid sequence of SEQ ID NO:81 and a VL comprising the amino acid sequence of SEQ ID NO:245.

In some such embodiments, the antibody or antigen binding fragment thereof comprises a VH comprising the amino acid sequence of SEQ ID NO:82 and a VL comprising the amino acid sequence of SEQ ID NO:245.

In some such embodiments, the antibody or antigen binding fragment thereof comprises a VH comprising the amino acid sequence of SEQ ID NO:83 and a VL comprising the amino acid sequence of SEQ ID NO:245.

In some such embodiments, the antibody or antigen binding fragment thereof comprises a VH comprising the amino acid sequence of SEQ ID NO:84 and a VL comprising the amino acid sequence of SEQ ID NO:245.

In some such embodiments, the antibody or antigen binding fragment thereof comprises a VH comprising the amino acid sequence of SEQ ID NO:85 and a VL comprising the amino acid sequence of SEQ ID NO:245.

In some such embodiments, the antibody or antigen binding fragment thereof comprises a VH comprising the amino acid sequence of SEQ ID NO:86 and a VL comprising the amino acid sequence of SEQ ID NO:245.

In some such embodiments, the antibody or antigen binding fragment thereof comprises a VH comprising the amino acid sequence of SEQ ID NO:87 and a VL comprising the amino acid sequence of SEQ ID NO:245.

In some such embodiments, the antibody or antigen binding fragment thereof comprises a VH comprising the amino acid sequence of SEQ ID NO:88 and a VL comprising the amino acid sequence of SEQ ID NO:245.

In some such embodiments, the antibody or antigen binding fragment thereof comprises a VH comprising the amino acid sequence of SEQ ID NO:238 and a VL comprising the amino acid sequence of SEQ ID NO:245.

In some such embodiments, the antibody or antigen binding fragment thereof comprises a VH comprising the amino acid sequence of SEQ ID NO:239 and a VL comprising the amino acid sequence of SEQ ID NO:245.

In some such embodiments, the antibody or antigen binding fragment thereof comprises a VH comprising the amino acid sequence of SEQ ID NO:240 and a VL comprising the amino acid sequence of SEQ ID NO:245.

In some such embodiments, the antibody or antigen binding fragment thereof comprises a VH comprising the amino acid sequence of SEQ ID NO:241 and a VL comprising the amino acid sequence of SEQ ID NO:245.

In some such embodiments, the antibody or antigen binding fragment thereof comprises a VH comprising the amino acid sequence of SEQ ID NO:242 and a VL comprising the amino acid sequence of SEQ ID NO:245.

In some such embodiments, the antibody or antigen binding fragment thereof comprises a VH comprising the amino acid sequence of SEQ ID NO:243 and a VL comprising the amino acid sequence of SEQ ID NO:245.

In some such embodiments, the antibody or antigen binding fragment thereof comprises a VH comprising the amino acid sequence of SEQ ID NO:43 and a VL comprising the amino acid sequence of SEQ ID NO:246.

In some such embodiments, the antibody or antigen binding fragment thereof comprises a VH comprising the amino acid sequence of SEQ ID NO:44 and a VL comprising the amino acid sequence of SEQ ID NO:246.

In some such embodiments, the antibody or antigen binding fragment thereof comprises a VH comprising the amino acid sequence of SEQ ID NO:45 and a VL comprising the amino acid sequence of SEQ ID NO:246.

In some such embodiments, the antibody or antigen binding fragment thereof comprises a VH comprising the amino acid sequence of SEQ ID NO:46 and a VL comprising the amino acid sequence of SEQ ID NO:246.

In some such embodiments, the antibody or antigen binding fragment thereof comprises a VH comprising the amino acid sequence of SEQ ID NO:47 and a VL comprising the amino acid sequence of SEQ ID NO:246.

In some such embodiments, the antibody or antigen binding fragment thereof comprises a VH comprising the amino acid sequence of SEQ ID NO:48 and a VL comprising the amino acid sequence of SEQ ID NO:246.

In some such embodiments, the antibody or antigen binding fragment thereof comprises a VH comprising the amino acid sequence of SEQ ID NO:49 and a VL comprising the amino acid sequence of SEQ ID NO:246.

In some such embodiments, the antibody or antigen binding fragment thereof comprises a VH comprising the amino acid sequence of SEQ ID NO:50 and a VL comprising the amino acid sequence of SEQ ID NO:246.

In some such embodiments, the antibody or antigen binding fragment thereof comprises a VH comprising the amino acid sequence of SEQ ID NO:51 and a VL comprising the amino acid sequence of SEQ ID NO:246.

In some such embodiments, the antibody or antigen binding fragment thereof comprises a VH comprising the amino acid sequence of SEQ ID NO:52 and a VL comprising the amino acid sequence of SEQ ID NO:246.

In some such embodiments, the antibody or antigen binding fragment thereof comprises a VH comprising the amino acid sequence of SEQ ID NO:53 and a VL comprising the amino acid sequence of SEQ ID NO:246.

In some such embodiments, the antibody or antigen binding fragment thereof comprises a VH comprising the amino acid sequence of SEQ ID NO:54 and a VL comprising the amino acid sequence of SEQ ID NO:246.

In some such embodiments, the antibody or antigen binding fragment thereof comprises a VH comprising the amino acid sequence of SEQ ID NO:55 and a VL comprising the amino acid sequence of SEQ ID NO:246.

In some such embodiments, the antibody or antigen binding fragment thereof comprises a VH comprising the amino acid sequence of SEQ ID NO:56 and a VL comprising the amino acid sequence of SEQ ID NO:246.

In some such embodiments, the antibody or antigen binding fragment thereof comprises a VH comprising the amino acid sequence of SEQ ID NO:57 and a VL comprising the amino acid sequence of SEQ ID NO:246.

In some such embodiments, the antibody or antigen binding fragment thereof comprises a VH comprising the amino acid sequence of SEQ ID NO:58 and a VL comprising the amino acid sequence of SEQ ID NO:246.

In some such embodiments, the antibody or antigen binding fragment thereof comprises a VH comprising the amino acid sequence of SEQ ID NO:59 and a VL comprising the amino acid sequence of SEQ ID NO:246.

In some such embodiments, the antibody or antigen binding fragment thereof comprises a VH comprising the amino acid sequence of SEQ ID NO:60 and a VL comprising the amino acid sequence of SEQ ID NO:246.

In some such embodiments, the antibody or antigen binding fragment thereof comprises a VH comprising the amino acid sequence of SEQ ID NO:61 and a VL comprising the amino acid sequence of SEQ ID NO:246.

In some such embodiments, the antibody or antigen binding fragment thereof comprises a VH comprising the amino acid sequence of SEQ ID NO:62 and a VL comprising the amino acid sequence of SEQ ID NO:246.

In some such embodiments, the antibody or antigen binding fragment thereof comprises a VH comprising the amino acid sequence of SEQ ID NO:63 and a VL comprising the amino acid sequence of SEQ ID NO:246.

In some such embodiments, the antibody or antigen binding fragment thereof comprises a VH comprising the amino acid sequence of SEQ ID NO:64 and a VL comprising the amino acid sequence of SEQ ID NO:246.

In some such embodiments, the antibody or antigen binding fragment thereof comprises a VH comprising the amino acid sequence of SEQ ID NO:65 and a VL comprising the amino acid sequence of SEQ ID NO:246.

In some such embodiments, the antibody or antigen binding fragment thereof comprises a VH comprising the amino acid sequence of SEQ ID NO:66 and a VL comprising the amino acid sequence of SEQ ID NO:246.

In some such embodiments, the antibody or antigen binding fragment thereof comprises a VH comprising the amino acid sequence of SEQ ID NO:67 and a VL comprising the amino acid sequence of SEQ ID NO:246.

In some such embodiments, the antibody or antigen binding fragment thereof comprises a VH comprising the amino acid sequence of SEQ ID NO:68 and a VL comprising the amino acid sequence of SEQ ID NO:246.

In some such embodiments, the antibody or antigen binding fragment thereof comprises a VH comprising the amino acid sequence of SEQ ID NO:69 and a VL comprising the amino acid sequence of SEQ ID NO:246.

In some such embodiments, the antibody or antigen binding fragment thereof comprises a VH comprising the amino acid sequence of SEQ ID NO:70 and a VL comprising the amino acid sequence of SEQ ID NO:246.

In some such embodiments, the antibody or antigen binding fragment thereof comprises a VH comprising the amino acid sequence of SEQ ID NO:71 and a VL comprising the amino acid sequence of SEQ ID NO:246.

In some such embodiments, the antibody or antigen binding fragment thereof comprises a VH comprising the amino acid sequence of SEQ ID NO:72 and a VL comprising the amino acid sequence of SEQ ID NO:246.

In some such embodiments, the antibody or antigen binding fragment thereof comprises a VH comprising the amino acid sequence of SEQ ID NO:73 and a VL comprising the amino acid sequence of SEQ ID NO:246.

In some such embodiments, the antibody or antigen binding fragment thereof comprises a VH comprising the amino acid sequence of SEQ ID NO:74 and a VL comprising the amino acid sequence of SEQ ID NO:246.

In some such embodiments, the antibody or antigen binding fragment thereof comprises a VH comprising the amino acid sequence of SEQ ID NO:75 and a VL comprising the amino acid sequence of SEQ ID NO:246.

In some such embodiments, the antibody or antigen binding fragment thereof comprises a VH comprising the amino acid sequence of SEQ ID NO:76 and a VL comprising the amino acid sequence of SEQ ID NO:246.

In some such embodiments, the antibody or antigen binding fragment thereof comprises a VH comprising the amino acid sequence of SEQ ID NO:77 and a VL comprising the amino acid sequence of SEQ ID NO:246.

In some such embodiments, the antibody or antigen binding fragment thereof comprises a VH comprising the amino acid sequence of SEQ ID NO:78 and a VL comprising the amino acid sequence of SEQ ID NO:246.

In some such embodiments, the antibody or antigen binding fragment thereof comprises a VH comprising the amino acid sequence of SEQ ID NO:79 and a VL comprising the amino acid sequence of SEQ ID NO:246.

In some such embodiments, the antibody or antigen binding fragment thereof comprises a VH comprising the amino acid sequence of SEQ ID NO:80 and a VL comprising the amino acid sequence of SEQ ID NO:246.

In some such embodiments, the antibody or antigen binding fragment thereof comprises a VH comprising the amino acid sequence of SEQ ID NO:81 and a VL comprising the amino acid sequence of SEQ ID NO:246.

In some such embodiments, the antibody or antigen binding fragment thereof comprises a VH comprising the amino acid sequence of SEQ ID NO:82 and a VL comprising the amino acid sequence of SEQ ID NO:246.

In some such embodiments, the antibody or antigen binding fragment thereof comprises a VH comprising the amino acid sequence of SEQ ID NO:83 and a VL comprising the amino acid sequence of SEQ ID NO:246.

In some such embodiments, the antibody or antigen binding fragment thereof comprises a VH comprising the amino acid sequence of SEQ ID NO:84 and a VL comprising the amino acid sequence of SEQ ID NO:246.

In some such embodiments, the antibody or antigen binding fragment thereof comprises a VH comprising the amino acid sequence of SEQ ID NO:85 and a VL comprising the amino acid sequence of SEQ ID NO:246.

In some such embodiments, the antibody or antigen binding fragment thereof comprises a VH comprising the amino acid sequence of SEQ ID NO:86 and a VL comprising the amino acid sequence of SEQ ID NO:246.

In some such embodiments, the antibody or antigen binding fragment thereof comprises a VH comprising the amino acid sequence of SEQ ID NO:87 and a VL comprising the amino acid sequence of SEQ ID NO:246.

In some such embodiments, the antibody or antigen binding fragment thereof comprises a VH comprising the amino acid sequence of SEQ ID NO:88 and a VL comprising the amino acid sequence of SEQ ID NO:246.

In some such embodiments, the antibody or antigen binding fragment thereof comprises a VH comprising the amino acid sequence of SEQ ID NO:238 and a VL comprising the amino acid sequence of SEQ ID NO:246.

In some such embodiments, the antibody or antigen binding fragment thereof comprises a VH comprising the amino acid sequence of SEQ ID NO:239 and a VL comprising the amino acid sequence of SEQ ID NO:246.

In some such embodiments, the antibody or antigen binding fragment thereof comprises a VH comprising the amino acid sequence of SEQ ID NO:240 and a VL comprising the amino acid sequence of SEQ ID NO:246.

In some such embodiments, the antibody or antigen binding fragment thereof comprises a VH comprising the amino acid sequence of SEQ ID NO:241 and a VL comprising the amino acid sequence of SEQ ID NO:246.

In some such embodiments, the antibody or antigen binding fragment thereof comprises a VH comprising the amino acid sequence of SEQ ID NO:242 and a VL comprising the amino acid sequence of SEQ ID NO:246.

In some such embodiments, the antibody or antigen binding fragment thereof comprises a VH comprising the amino acid sequence of SEQ ID NO:243 and a VL comprising the amino acid sequence of SEQ ID NO:246.

In some such embodiments, the antibody or antigen binding fragment thereof comprises a VH comprising the amino acid sequence of SEQ ID NO:43 and a VL comprising the amino acid sequence of SEQ ID NO:247.

In some such embodiments, the antibody or antigen binding fragment thereof comprises a VH comprising the amino acid sequence of SEQ ID NO:44 and a VL comprising the amino acid sequence of SEQ ID NO:247.

In some such embodiments, the antibody or antigen binding fragment thereof comprises a VH comprising the amino acid sequence of SEQ ID NO:45 and a VL comprising the amino acid sequence of SEQ ID NO:247.

In some such embodiments, the antibody or antigen binding fragment thereof comprises a VH comprising the amino acid sequence of SEQ ID NO:46 and a VL comprising the amino acid sequence of SEQ ID NO:247.

In some such embodiments, the antibody or antigen binding fragment thereof comprises a VH comprising the amino acid sequence of SEQ ID NO:47 and a VL comprising the amino acid sequence of SEQ ID NO:247.

In some such embodiments, the antibody or antigen binding fragment thereof comprises a VH comprising the amino acid sequence of SEQ ID NO:48 and a VL comprising the amino acid sequence of SEQ ID NO:247.

In some such embodiments, the antibody or antigen binding fragment thereof comprises a VH comprising the amino acid sequence of SEQ ID NO:49 and a VL comprising the amino acid sequence of SEQ ID NO:247.

In some such embodiments, the antibody or antigen binding fragment thereof comprises a VH comprising the amino acid sequence of SEQ ID NO:50 and a VL comprising the amino acid sequence of SEQ ID NO:247.

In some such embodiments, the antibody or antigen binding fragment thereof comprises a VH comprising the amino acid sequence of SEQ ID NO:51 and a VL comprising the amino acid sequence of SEQ ID NO:247.

In some such embodiments, the antibody or antigen binding fragment thereof comprises a VH comprising the amino acid sequence of SEQ ID NO:52 and a VL comprising the amino acid sequence of SEQ ID NO:247.

In some such embodiments, the antibody or antigen binding fragment thereof comprises a VH comprising the amino acid sequence of SEQ ID NO:53 and a VL comprising the amino acid sequence of SEQ ID NO:247.

In some such embodiments, the antibody or antigen binding fragment thereof comprises a VH comprising the amino acid sequence of SEQ ID NO:54 and a VL comprising the amino acid sequence of SEQ ID NO:247.

In some such embodiments, the antibody or antigen binding fragment thereof comprises a VH comprising the amino acid sequence of SEQ ID NO:55 and a VL comprising the amino acid sequence of SEQ ID NO:247.

In some such embodiments, the antibody or antigen binding fragment thereof comprises a VH comprising the amino acid sequence of SEQ ID NO:56 and a VL comprising the amino acid sequence of SEQ ID NO:247.

In some such embodiments, the antibody or antigen binding fragment thereof comprises a VH comprising the amino acid sequence of SEQ ID NO:57 and a VL comprising the amino acid sequence of SEQ ID NO:247.

In some such embodiments, the antibody or antigen binding fragment thereof comprises a VH comprising the amino acid sequence of SEQ ID NO:58 and a VL comprising the amino acid sequence of SEQ ID NO:247.

In some such embodiments, the antibody or antigen binding fragment thereof comprises a VH comprising the amino acid sequence of SEQ ID NO:59 and a VL comprising the amino acid sequence of SEQ ID NO:247.

In some such embodiments, the antibody or antigen binding fragment thereof comprises a VH comprising the amino acid sequence of SEQ ID NO:60 and a VL comprising the amino acid sequence of SEQ ID NO:247.

In some such embodiments, the antibody or antigen binding fragment thereof comprises a VH comprising the amino acid sequence of SEQ ID NO:61 and a VL comprising the amino acid sequence of SEQ ID NO:247.

In some such embodiments, the antibody or antigen binding fragment thereof comprises a VH comprising the amino acid sequence of SEQ ID NO:62 and a VL comprising the amino acid sequence of SEQ ID NO:247.

In some such embodiments, the antibody or antigen binding fragment thereof comprises a VH comprising the amino acid sequence of SEQ ID NO:63 and a VL comprising the amino acid sequence of SEQ ID NO:247.

In some such embodiments, the antibody or antigen binding fragment thereof comprises a VH comprising the amino acid sequence of SEQ ID NO:64 and a VL comprising the amino acid sequence of SEQ ID NO:247.

In some such embodiments, the antibody or antigen binding fragment thereof comprises a VH comprising the amino acid sequence of SEQ ID NO:65 and a VL comprising the amino acid sequence of SEQ ID NO:247.

In some such embodiments, the antibody or antigen binding fragment thereof comprises a VH comprising the amino acid sequence of SEQ ID NO:66 and a VL comprising the amino acid sequence of SEQ ID NO:247.

In some such embodiments, the antibody or antigen binding fragment thereof comprises a VH comprising the amino acid sequence of SEQ ID NO:67 and a VL comprising the amino acid sequence of SEQ ID NO:247.

In some such embodiments, the antibody or antigen binding fragment thereof comprises a VH comprising the amino acid sequence of SEQ ID NO:68 and a VL comprising the amino acid sequence of SEQ ID NO:247.

In some such embodiments, the antibody or antigen binding fragment thereof comprises a VH comprising the amino acid sequence of SEQ ID NO:69 and a VL comprising the amino acid sequence of SEQ ID NO:247.

In some such embodiments, the antibody or antigen binding fragment thereof comprises a VH comprising the amino acid sequence of SEQ ID NO:70 and a VL comprising the amino acid sequence of SEQ ID NO:247.

In some such embodiments, the antibody or antigen binding fragment thereof comprises a VH comprising the amino acid sequence of SEQ ID NO:71 and a VL comprising the amino acid sequence of SEQ ID NO:247.

In some such embodiments, the antibody or antigen binding fragment thereof comprises a VH comprising the amino acid sequence of SEQ ID NO:72 and a VL comprising the amino acid sequence of SEQ ID NO:247.

In some such embodiments, the antibody or antigen binding fragment thereof comprises a VH comprising the amino acid sequence of SEQ ID NO:73 and a VL comprising the amino acid sequence of SEQ ID NO:247.

In some such embodiments, the antibody or antigen binding fragment thereof comprises a VH comprising the amino acid sequence of SEQ ID NO:74 and a VL comprising the amino acid sequence of SEQ ID NO:247.

In some such embodiments, the antibody or antigen binding fragment thereof comprises a VH comprising the amino acid sequence of SEQ ID NO:75 and a VL comprising the amino acid sequence of SEQ ID NO:247.

In some such embodiments, the antibody or antigen binding fragment thereof comprises a VH comprising the amino acid sequence of SEQ ID NO:76 and a VL comprising the amino acid sequence of SEQ ID NO:247.

In some such embodiments, the antibody or antigen binding fragment thereof comprises a VH comprising the amino acid sequence of SEQ ID NO:77 and a VL comprising the amino acid sequence of SEQ ID NO:247.

In some such embodiments, the antibody or antigen binding fragment thereof comprises a VH comprising the amino acid sequence of SEQ ID NO:78 and a VL comprising the amino acid sequence of SEQ ID NO:247.

In some such embodiments, the antibody or antigen binding fragment thereof comprises a VH comprising the amino acid sequence of SEQ ID NO:79 and a VL comprising the amino acid sequence of SEQ ID NO:247.

In some such embodiments, the antibody or antigen binding fragment thereof comprises a VH comprising the amino acid sequence of SEQ ID NO:80 and a VL comprising the amino acid sequence of SEQ ID NO:247.

In some such embodiments, the antibody or antigen binding fragment thereof comprises a VH comprising the amino acid sequence of SEQ ID NO:81 and a VL comprising the amino acid sequence of SEQ ID NO:247.

In some such embodiments, the antibody or antigen binding fragment thereof comprises a VH comprising the amino acid sequence of SEQ ID NO:82 and a VL comprising the amino acid sequence of SEQ ID NO:247.

In some such embodiments, the antibody or antigen binding fragment thereof comprises a VH comprising the amino acid sequence of SEQ ID NO:83 and a VL comprising the amino acid sequence of SEQ ID NO:247.

In some such embodiments, the antibody or antigen binding fragment thereof comprises a VH comprising the amino acid sequence of SEQ ID NO:84 and a VL comprising the amino acid sequence of SEQ ID NO:247.

In some such embodiments, the antibody or antigen binding fragment thereof comprises a VH comprising the amino acid sequence of SEQ ID NO:85 and a VL comprising the amino acid sequence of SEQ ID NO:247.

In some such embodiments, the antibody or antigen binding fragment thereof comprises a VH comprising the amino acid sequence of SEQ ID NO:86 and a VL comprising the amino acid sequence of SEQ ID NO:247.

In some such embodiments, the antibody or antigen binding fragment thereof comprises a VH comprising the amino acid sequence of SEQ ID NO:87 and a VL comprising the amino acid sequence of SEQ ID NO:247.

In some such embodiments, the antibody or antigen binding fragment thereof comprises a VH comprising the amino acid sequence of SEQ ID NO:88 and a VL comprising the amino acid sequence of SEQ ID NO:247.

In some such embodiments, the antibody or antigen binding fragment thereof comprises a VH comprising the amino acid sequence of SEQ ID NO:238 and a VL comprising the amino acid sequence of SEQ ID NO:247.

In some such embodiments, the antibody or antigen binding fragment thereof comprises a VH comprising the amino acid sequence of SEQ ID NO:239 and a VL comprising the amino acid sequence of SEQ ID NO:247.

In some such embodiments, the antibody or antigen binding fragment thereof comprises a VH comprising the amino acid sequence of SEQ ID NO:240 and a VL comprising the amino acid sequence of SEQ ID NO:247.

In some such embodiments, the antibody or antigen binding fragment thereof comprises a VH comprising the amino acid sequence of SEQ ID NO:241 and a VL comprising the amino acid sequence of SEQ ID NO:247.

In some such embodiments, the antibody or antigen binding fragment thereof comprises a VH comprising the amino acid sequence of SEQ ID NO:242 and a VL comprising the amino acid sequence of SEQ ID NO:247.

In some such embodiments, the antibody or antigen binding fragment thereof comprises a VH comprising the amino acid sequence of SEQ ID NO:243 and a VL comprising the amino acid sequence of SEQ ID NO:247.

In some such embodiments, the antibody or antigen binding fragment thereof comprises a VH comprising the amino acid sequence of SEQ ID NO:43 and a VL comprising the amino acid sequence of SEQ ID NO:248.

In some such embodiments, the antibody or antigen binding fragment thereof comprises a VH comprising the amino acid sequence of SEQ ID NO:44 and a VL comprising the amino acid sequence of SEQ ID NO:248.

In some such embodiments, the antibody or antigen binding fragment thereof comprises a VH comprising the amino acid sequence of SEQ ID NO:45 and a VL comprising the amino acid sequence of SEQ ID NO:248.

In some such embodiments, the antibody or antigen binding fragment thereof comprises a VH comprising the amino acid sequence of SEQ ID NO:46 and a VL comprising the amino acid sequence of SEQ ID NO:248.

In some such embodiments, the antibody or antigen binding fragment thereof comprises a VH comprising the amino acid sequence of SEQ ID NO:47 and a VL comprising the amino acid sequence of SEQ ID NO:248.

In some such embodiments, the antibody or antigen binding fragment thereof comprises a VH comprising the amino acid sequence of SEQ ID NO:48 and a VL comprising the amino acid sequence of SEQ ID NO:248.

In some such embodiments, the antibody or antigen binding fragment thereof comprises a VH comprising the amino acid sequence of SEQ ID NO:49 and a VL comprising the amino acid sequence of SEQ ID NO:248.

In some such embodiments, the antibody or antigen binding fragment thereof comprises a VH comprising the amino acid sequence of SEQ ID NO:50 and a VL comprising the amino acid sequence of SEQ ID NO:248.

In some such embodiments, the antibody or antigen binding fragment thereof comprises a VH comprising the amino acid sequence of SEQ ID NO:51 and a VL comprising the amino acid sequence of SEQ ID NO:248.

In some such embodiments, the antibody or antigen binding fragment thereof comprises a VH comprising the amino acid sequence of SEQ ID NO:52 and a VL comprising the amino acid sequence of SEQ ID NO:248.

In some such embodiments, the antibody or antigen binding fragment thereof comprises a VH comprising the amino acid sequence of SEQ ID NO:53 and a VL comprising the amino acid sequence of SEQ ID NO:248.

In some such embodiments, the antibody or antigen binding fragment thereof comprises a VH comprising the amino acid sequence of SEQ ID NO:54 and a VL comprising the amino acid sequence of SEQ ID NO:248.

In some such embodiments, the antibody or antigen binding fragment thereof comprises a VH comprising the amino acid sequence of SEQ ID NO:55 and a VL comprising the amino acid sequence of SEQ ID NO:248.

In some such embodiments, the antibody or antigen binding fragment thereof comprises a VH comprising the amino acid sequence of SEQ ID NO:56 and a VL comprising the amino acid sequence of SEQ ID NO:248.

In some such embodiments, the antibody or antigen binding fragment thereof comprises a VH comprising the amino acid sequence of SEQ ID NO:57 and a VL comprising the amino acid sequence of SEQ ID NO:248.

In some such embodiments, the antibody or antigen binding fragment thereof comprises a VH comprising the amino acid sequence of SEQ ID NO:58 and a VL comprising the amino acid sequence of SEQ ID NO:248.

In some such embodiments, the antibody or antigen binding fragment thereof comprises a VH comprising the amino acid sequence of SEQ ID NO:59 and a VL comprising the amino acid sequence of SEQ ID NO:248.

In some such embodiments, the antibody or antigen binding fragment thereof comprises a VH comprising the amino acid sequence of SEQ ID NO:60 and a VL comprising the amino acid sequence of SEQ ID NO:248.

In some such embodiments, the antibody or antigen binding fragment thereof comprises a VH comprising the amino acid sequence of SEQ ID NO:61 and a VL comprising the amino acid sequence of SEQ ID NO:248.

In some such embodiments, the antibody or antigen binding fragment thereof comprises a VH comprising the amino acid sequence of SEQ ID NO:62 and a VL comprising the amino acid sequence of SEQ ID NO:248.

In some such embodiments, the antibody or antigen binding fragment thereof comprises a VH comprising the amino acid sequence of SEQ ID NO:63 and a VL comprising the amino acid sequence of SEQ ID NO:248.

In some such embodiments, the antibody or antigen binding fragment thereof comprises a VH comprising the amino acid sequence of SEQ ID NO:64 and a VL comprising the amino acid sequence of SEQ ID NO:248.

In some such embodiments, the antibody or antigen binding fragment thereof comprises a VH comprising the amino acid sequence of SEQ ID NO:65 and a VL comprising the amino acid sequence of SEQ ID NO:248.

In some such embodiments, the antibody or antigen binding fragment thereof comprises a VH comprising the amino acid sequence of SEQ ID NO:66 and a VL comprising the amino acid sequence of SEQ ID NO:248.

In some such embodiments, the antibody or antigen binding fragment thereof comprises a VH comprising the amino acid sequence of SEQ ID NO:67 and a VL comprising the amino acid sequence of SEQ ID NO:248.

In some such embodiments, the antibody or antigen binding fragment thereof comprises a VH comprising the amino acid sequence of SEQ ID NO:68 and a VL comprising the amino acid sequence of SEQ ID NO:248.

In some such embodiments, the antibody or antigen binding fragment thereof comprises a VH comprising the amino acid sequence of SEQ ID NO:69 and a VL comprising the amino acid sequence of SEQ ID NO:248.

In some such embodiments, the antibody or antigen binding fragment thereof comprises a VH comprising the amino acid sequence of SEQ ID NO:70 and a VL comprising the amino acid sequence of SEQ ID NO:248.

In some such embodiments, the antibody or antigen binding fragment thereof comprises a VH comprising the amino acid sequence of SEQ ID NO:71 and a VL comprising the amino acid sequence of SEQ ID NO:248.

In some such embodiments, the antibody or antigen binding fragment thereof comprises a VH comprising the amino acid sequence of SEQ ID NO:72 and a VL comprising the amino acid sequence of SEQ ID NO:248.

In some such embodiments, the antibody or antigen binding fragment thereof comprises a VH comprising the amino acid sequence of SEQ ID NO:73 and a VL comprising the amino acid sequence of SEQ ID NO:248.

In some such embodiments, the antibody or antigen binding fragment thereof comprises a VH comprising the amino acid sequence of SEQ ID NO:74 and a VL comprising the amino acid sequence of SEQ ID NO:248.

In some such embodiments, the antibody or antigen binding fragment thereof comprises a VH comprising the amino acid sequence of SEQ ID NO:75 and a VL comprising the amino acid sequence of SEQ ID NO:248.

In some such embodiments, the antibody or antigen binding fragment thereof comprises a VH comprising the amino acid sequence of SEQ ID NO:76 and a VL comprising the amino acid sequence of SEQ ID NO:248.

In some such embodiments, the antibody or antigen binding fragment thereof comprises a VH comprising the amino acid sequence of SEQ ID NO:77 and a VL comprising the amino acid sequence of SEQ ID NO:248.

In some such embodiments, the antibody or antigen binding fragment thereof comprises a VH comprising the amino acid sequence of SEQ ID NO:78 and a VL comprising the amino acid sequence of SEQ ID NO:248.

In some such embodiments, the antibody or antigen binding fragment thereof comprises a VH comprising the amino acid sequence of SEQ ID NO:79 and a VL comprising the amino acid sequence of SEQ ID NO:248.

In some such embodiments, the antibody or antigen binding fragment thereof comprises a VH comprising the amino acid sequence of SEQ ID NO:80 and a VL comprising the amino acid sequence of SEQ ID NO:248.

In some such embodiments, the antibody or antigen binding fragment thereof comprises a VH comprising the amino acid sequence of SEQ ID NO:81 and a VL comprising the amino acid sequence of SEQ ID NO:248.

In some such embodiments, the antibody or antigen binding fragment thereof comprises a VH comprising the amino acid sequence of SEQ ID NO:82 and a VL comprising the amino acid sequence of SEQ ID NO:248.

In some such embodiments, the antibody or antigen binding fragment thereof comprises a VH comprising the amino acid sequence of SEQ ID NO:83 and a VL comprising the amino acid sequence of SEQ ID NO:248.

In some such embodiments, the antibody or antigen binding fragment thereof comprises a VH comprising the amino acid sequence of SEQ ID NO:84 and a VL comprising the amino acid sequence of SEQ ID NO:248.

In some such embodiments, the antibody or antigen binding fragment thereof comprises a VH comprising the amino acid sequence of SEQ ID NO:85 and a VL comprising the amino acid sequence of SEQ ID NO:248.

In some such embodiments, the antibody or antigen binding fragment thereof comprises a VH comprising the amino acid sequence of SEQ ID NO:86 and a VL comprising the amino acid sequence of SEQ ID NO:248.

In some such embodiments, the antibody or antigen binding fragment thereof comprises a VH comprising the amino acid sequence of SEQ ID NO:87 and a VL comprising the amino acid sequence of SEQ ID NO:248.

In some such embodiments, the antibody or antigen binding fragment thereof comprises a VH comprising the amino acid sequence of SEQ ID NO:88 and a VL comprising the amino acid sequence of SEQ ID NO:248.

In some such embodiments, the antibody or antigen binding fragment thereof comprises a VH comprising the amino acid sequence of SEQ ID NO:238 and a VL comprising the amino acid sequence of SEQ ID NO:248.

In some such embodiments, the antibody or antigen binding fragment thereof comprises a VH comprising the amino acid sequence of SEQ ID NO:239 and a VL comprising the amino acid sequence of SEQ ID NO:248.

In some such embodiments, the antibody or antigen binding fragment thereof comprises a VH comprising the amino acid sequence of SEQ ID NO:240 and a VL comprising the amino acid sequence of SEQ ID NO:248.

In some such embodiments, the antibody or antigen binding fragment thereof comprises a VH comprising the amino acid sequence of SEQ ID NO:241 and a VL comprising the amino acid sequence of SEQ ID NO:248.

In some such embodiments, the antibody or antigen binding fragment thereof comprises a VH comprising the amino acid sequence of SEQ ID NO:242 and a VL comprising the amino acid sequence of SEQ ID NO:248.

In some such embodiments, the antibody or antigen binding fragment thereof comprises a VH comprising the amino acid sequence of SEQ ID NO:243 and a VL comprising the amino acid sequence of SEQ ID NO:248.

In some such embodiments, the antibody or antigen binding fragment thereof comprises a VH comprising the amino acid sequence of SEQ ID NO:43 and a VL comprising the amino acid sequence of SEQ ID NO:249.

In some such embodiments, the antibody or antigen binding fragment thereof comprises a VH comprising the amino acid sequence of SEQ ID NO:44 and a VL comprising the amino acid sequence of SEQ ID NO:249.

In some such embodiments, the antibody or antigen binding fragment thereof comprises a VH comprising the amino acid sequence of SEQ ID NO:45 and a VL comprising the amino acid sequence of SEQ ID NO:249.

In some such embodiments, the antibody or antigen binding fragment thereof comprises a VH comprising the amino acid sequence of SEQ ID NO:46 and a VL comprising the amino acid sequence of SEQ ID NO:249.

In some such embodiments, the antibody or antigen binding fragment thereof comprises a VH comprising the amino acid sequence of SEQ ID NO:47 and a VL comprising the amino acid sequence of SEQ ID NO:249.

In some such embodiments, the antibody or antigen binding fragment thereof comprises a VH comprising the amino acid sequence of SEQ ID NO:48 and a VL comprising the amino acid sequence of SEQ ID NO:249.

In some such embodiments, the antibody or antigen binding fragment thereof comprises a VH comprising the amino acid sequence of SEQ ID NO:49 and a VL comprising the amino acid sequence of SEQ ID NO:249.

In some such embodiments, the antibody or antigen binding fragment thereof comprises a VH comprising the amino acid sequence of SEQ ID NO:50 and a VL comprising the amino acid sequence of SEQ ID NO:249.

In some such embodiments, the antibody or antigen binding fragment thereof comprises a VH comprising the amino acid sequence of SEQ ID NO:51 and a VL comprising the amino acid sequence of SEQ ID NO:249.

In some such embodiments, the antibody or antigen binding fragment thereof comprises a VH comprising the amino acid sequence of SEQ ID NO:52 and a VL comprising the amino acid sequence of SEQ ID NO:249.

In some such embodiments, the antibody or antigen binding fragment thereof comprises a VH comprising the amino acid sequence of SEQ ID NO:53 and a VL comprising the amino acid sequence of SEQ ID NO:249.

In some such embodiments, the antibody or antigen binding fragment thereof comprises a VH comprising the amino acid sequence of SEQ ID NO:54 and a VL comprising the amino acid sequence of SEQ ID NO:249.

In some such embodiments, the antibody or antigen binding fragment thereof comprises a VH comprising the amino acid sequence of SEQ ID NO:55 and a VL comprising the amino acid sequence of SEQ ID NO:249.

In some such embodiments, the antibody or antigen binding fragment thereof comprises a VH comprising the amino acid sequence of SEQ ID NO:56 and a VL comprising the amino acid sequence of SEQ ID NO:249.

In some such embodiments, the antibody or antigen binding fragment thereof comprises a VH comprising the amino acid sequence of SEQ ID NO:57 and a VL comprising the amino acid sequence of SEQ ID NO:249.

In some such embodiments, the antibody or antigen binding fragment thereof comprises a VH comprising the amino acid sequence of SEQ ID NO:58 and a VL comprising the amino acid sequence of SEQ ID NO:249.

In some such embodiments, the antibody or antigen binding fragment thereof comprises a VH comprising the amino acid sequence of SEQ ID NO:59 and a VL comprising the amino acid sequence of SEQ ID NO:249.

In some such embodiments, the antibody or antigen binding fragment thereof comprises a VH comprising the amino acid sequence of SEQ ID NO:60 and a VL comprising the amino acid sequence of SEQ ID NO:249.

In some such embodiments, the antibody or antigen binding fragment thereof comprises a VH comprising the amino acid sequence of SEQ ID NO:61 and a VL comprising the amino acid sequence of SEQ ID NO:249.

In some such embodiments, the antibody or antigen binding fragment thereof comprises a VH comprising the amino acid sequence of SEQ ID NO:62 and a VL comprising the amino acid sequence of SEQ ID NO:249.

In some such embodiments, the antibody or antigen binding fragment thereof comprises a VH comprising the amino acid sequence of SEQ ID NO:63 and a VL comprising the amino acid sequence of SEQ ID NO:249.

In some such embodiments, the antibody or antigen binding fragment thereof comprises a VH comprising the amino acid sequence of SEQ ID NO:64 and a VL comprising the amino acid sequence of SEQ ID NO:249.

In some such embodiments, the antibody or antigen binding fragment thereof comprises a VH comprising the amino acid sequence of SEQ ID NO:65 and a VL comprising the amino acid sequence of SEQ ID NO:249.

In some such embodiments, the antibody or antigen binding fragment thereof comprises a VH comprising the amino acid sequence of SEQ ID NO:66 and a VL comprising the amino acid sequence of SEQ ID NO:249.

In some such embodiments, the antibody or antigen binding fragment thereof comprises a VH comprising the amino acid sequence of SEQ ID NO:67 and a VL comprising the amino acid sequence of SEQ ID NO:249.

In some such embodiments, the antibody or antigen binding fragment thereof comprises a VH comprising the amino acid sequence of SEQ ID NO:68 and a VL comprising the amino acid sequence of SEQ ID NO:249.

In some such embodiments, the antibody or antigen binding fragment thereof comprises a VH comprising the amino acid sequence of SEQ ID NO:69 and a VL comprising the amino acid sequence of SEQ ID NO:249.

In some such embodiments, the antibody or antigen binding fragment thereof comprises a VH comprising the amino acid sequence of SEQ ID NO:70 and a VL comprising the amino acid sequence of SEQ ID NO:249.

In some such embodiments, the antibody or antigen binding fragment thereof comprises a VH comprising the amino acid sequence of SEQ ID NO:71 and a VL comprising the amino acid sequence of SEQ ID NO:249.

In some such embodiments, the antibody or antigen binding fragment thereof comprises a VH comprising the amino acid sequence of SEQ ID NO:72 and a VL comprising the amino acid sequence of SEQ ID NO:249.

In some such embodiments, the antibody or antigen binding fragment thereof comprises a VH comprising the amino acid sequence of SEQ ID NO:73 and a VL comprising the amino acid sequence of SEQ ID NO:249.

In some such embodiments, the antibody or antigen binding fragment thereof comprises a VH comprising the amino acid sequence of SEQ ID NO:74 and a VL comprising the amino acid sequence of SEQ ID NO:249.

In some such embodiments, the antibody or antigen binding fragment thereof comprises a VH comprising the amino acid sequence of SEQ ID NO:75 and a VL comprising the amino acid sequence of SEQ ID NO:249.

In some such embodiments, the antibody or antigen binding fragment thereof comprises a VH comprising the amino acid sequence of SEQ ID NO:76 and a VL comprising the amino acid sequence of SEQ ID NO:249.

In some such embodiments, the antibody or antigen binding fragment thereof comprises a VH comprising the amino acid sequence of SEQ ID NO:77 and a VL comprising the amino acid sequence of SEQ ID NO:249.

In some such embodiments, the antibody or antigen binding fragment thereof comprises a VH comprising the amino acid sequence of SEQ ID NO:78 and a VL comprising the amino acid sequence of SEQ ID NO:249.

In some such embodiments, the antibody or antigen binding fragment thereof comprises a VH comprising the amino acid sequence of SEQ ID NO:79 and a VL comprising the amino acid sequence of SEQ ID NO:249.

In some such embodiments, the antibody or antigen binding fragment thereof comprises a VH comprising the amino acid sequence of SEQ ID NO:80 and a VL comprising the amino acid sequence of SEQ ID NO:249.

In some such embodiments, the antibody or antigen binding fragment thereof comprises a VH comprising the amino acid sequence of SEQ ID NO:81 and a VL comprising the amino acid sequence of SEQ ID NO:249.

In some such embodiments, the antibody or antigen binding fragment thereof comprises a VH comprising the amino acid sequence of SEQ ID NO:82 and a VL comprising the amino acid sequence of SEQ ID NO:249.

In some such embodiments, the antibody or antigen binding fragment thereof comprises a VH comprising the amino acid sequence of SEQ ID NO:83 and a VL comprising the amino acid sequence of SEQ ID NO:249.

In some such embodiments, the antibody or antigen binding fragment thereof comprises a VH comprising the amino acid sequence of SEQ ID NO:84 and a VL comprising the amino acid sequence of SEQ ID NO:249.

In some such embodiments, the antibody or antigen binding fragment thereof comprises a VH comprising the amino acid sequence of SEQ ID NO:85 and a VL comprising the amino acid sequence of SEQ ID NO:249.

In some such embodiments, the antibody or antigen binding fragment thereof comprises a VH comprising the amino acid sequence of SEQ ID NO:86 and a VL comprising the amino acid sequence of SEQ ID NO:249.

In some such embodiments, the antibody or antigen binding fragment thereof comprises a VH comprising the amino acid sequence of SEQ ID NO:87 and a VL comprising the amino acid sequence of SEQ ID NO:249.

In some such embodiments, the antibody or antigen binding fragment thereof comprises a VH comprising the amino acid sequence of SEQ ID NO:88 and a VL comprising the amino acid sequence of SEQ ID NO:249.

In some such embodiments, the antibody or antigen binding fragment thereof comprises a VH comprising the amino acid sequence of SEQ ID NO:238 and a VL comprising the amino acid sequence of SEQ ID NO:249.

In some such embodiments, the antibody or antigen binding fragment thereof comprises a VH comprising the amino acid sequence of SEQ ID NO:239 and a VL comprising the amino acid sequence of SEQ ID NO:249.

In some such embodiments, the antibody or antigen binding fragment thereof comprises a VH comprising the amino acid sequence of SEQ ID NO:240 and a VL comprising the amino acid sequence of SEQ ID NO:249.

In some such embodiments, the antibody or antigen binding fragment thereof comprises a VH comprising the amino acid sequence of SEQ ID NO:241 and a VL comprising the amino acid sequence of SEQ ID NO:249.

In some such embodiments, the antibody or antigen binding fragment thereof comprises a VH comprising the amino acid sequence of SEQ ID NO:242 and a VL comprising the amino acid sequence of SEQ ID NO:249.

In some such embodiments, the antibody or antigen binding fragment thereof comprises a VH comprising the amino acid sequence of SEQ ID NO:243 and a VL comprising the amino acid sequence of SEQ ID NO:249.

In some such embodiments, the antibody or antigen binding fragment thereof comprises a VH comprising the amino acid sequence of SEQ ID NO:39, and a VL comprising the amino acid sequence of SEQ ID NO:127.

In some such embodiments, the antibody or antigen binding fragment thereof comprises a VH comprising the amino acid sequence selected from any one of SEQ ID NOS:89-93, and a VL comprising the amino acid sequence selected from any one of SEQ ID NOS:134-137.

In some such embodiments, the antibody or antigen binding fragment thereof comprises a VH comprising the amino acid sequence of SEQ ID NO:89 and a VL comprising the amino acid sequence of SEQ ID NO:134.

In some such embodiments, the antibody or antigen binding fragment thereof comprises a VH comprising the amino acid sequence of SEQ ID NO:90 and a VL comprising the amino acid sequence of SEQ ID NO:134.

In some such embodiments, the antibody or antigen binding fragment thereof comprises a VH comprising the amino acid sequence of SEQ ID NO:91 and a VL comprising the amino acid sequence of SEQ ID NO:134.

In some such embodiments, the antibody or antigen binding fragment thereof comprises a VH comprising the amino acid sequence of SEQ ID NO:92 and a VL comprising the amino acid sequence of SEQ ID NO:134.

In some such embodiments, the antibody or antigen binding fragment thereof comprises a VH comprising the amino acid sequence of SEQ ID NO:93 and a VL comprising the amino acid sequence of SEQ ID NO:134.

In some such embodiments, the antibody or antigen binding fragment thereof comprises a VH comprising the amino acid sequence of SEQ ID NO:89 and a VL comprising the amino acid sequence of SEQ ID NO:135.

In some such embodiments, the antibody or antigen binding fragment thereof comprises a VH comprising the amino acid sequence of SEQ ID NO:90 and a VL comprising the amino acid sequence of SEQ ID NO:135.

In some such embodiments, the antibody or antigen binding fragment thereof comprises a VH comprising the amino acid sequence of SEQ ID NO:91 and a VL comprising the amino acid sequence of SEQ ID NO:135.

In some such embodiments, the antibody or antigen binding fragment thereof comprises a VH comprising the amino acid sequence of SEQ ID NO:92 and a VL comprising the amino acid sequence of SEQ ID NO:135.

In some such embodiments, the antibody or antigen binding fragment thereof comprises a VH comprising the amino acid sequence of SEQ ID NO:93 and a VL comprising the amino acid sequence of SEQ ID NO:135.

In some such embodiments, the antibody or antigen binding fragment thereof comprises a VH comprising the amino acid sequence of SEQ ID NO:89 and a VL comprising the amino acid sequence of SEQ ID NO:136.

In some such embodiments, the antibody or antigen binding fragment thereof comprises a VH comprising the amino acid sequence of SEQ ID NO:90 and a VL comprising the amino acid sequence of SEQ ID NO:136.

In some such embodiments, the antibody or antigen binding fragment thereof comprises a VH comprising the amino acid sequence of SEQ ID NO:91 and a VL comprising the amino acid sequence of SEQ ID NO:136.

In some such embodiments, the antibody or antigen binding fragment thereof comprises a VH comprising the amino acid sequence of SEQ ID NO:92 and a VL comprising the amino acid sequence of SEQ ID NO:136.

In some such embodiments, the antibody or antigen binding fragment thereof comprises a VH comprising the amino acid sequence of SEQ ID NO:93 and a VL comprising the amino acid sequence of SEQ ID NO:136.

In some such embodiments, the antibody or antigen binding fragment thereof comprises a VH comprising the amino acid sequence of SEQ ID NO:89 and a VL comprising the amino acid sequence of SEQ ID NO:137.

In some such embodiments, the antibody or antigen binding fragment thereof comprises a VH comprising the amino acid sequence of SEQ ID NO:90 and a VL comprising the amino acid sequence of SEQ ID NO:137.

In some such embodiments, the antibody or antigen binding fragment thereof comprises a VH comprising the amino acid sequence of SEQ ID NO:91 and a VL comprising the amino acid sequence of SEQ ID NO:137.

In some such embodiments, the antibody or antigen binding fragment thereof comprises a VH comprising the amino acid sequence of SEQ ID NO:92 and a VL comprising the amino acid sequence of SEQ ID NO:137.

In some such embodiments, the antibody or antigen binding fragment thereof comprises a VH comprising the amino acid sequence of SEQ ID NO:93 and a VL comprising the amino acid sequence of SEQ ID NO:137.

In some such embodiments, the antibody or antigen binding fragment thereof comprises a VH comprising the amino acid sequence of SEQ ID NO:42, and a VL comprising the amino acid sequence of SEQ ID NO:130.

In some such embodiments, the antibody or antigen binding fragment thereof comprises a VH comprising the amino acid sequence selected from any one of SEQ ID NOS:113-125, and a VL comprising the amino acid sequence selected from any one of SEQ ID NOS:143-149.

In some such embodiments, the antibody or antigen binding fragment thereof comprises a VH comprising the amino acid sequence of SEQ ID NO:113 and a VL comprising the amino acid sequence of SEQ ID NO:143.

In some such embodiments, the antibody or antigen binding fragment thereof comprises a VH comprises the amino acid sequence of SEQ ID NO:121 and a VL comprising the amino acid sequence of SEQ ID NO:143.

In some such embodiments, the antibody or antigen binding fragment thereof comprises a VH comprising the amino acid sequence of SEQ ID NO:118 and a VL comprising the amino acid sequence of SEQ ID NO:143.

In some such embodiments, the antibody or antigen binding fragment thereof comprises a VH comprising the amino acid sequence of SEQ ID NO:122 and a VL comprising the amino acid sequence of SEQ ID NO:143.

In some such embodiments, the antibody or antigen binding fragment thereof comprises a VH comprising the amino acid sequence of SEQ ID NO:123 and a VL comprising the amino acid sequence of SEQ ID NO:143.

In some such embodiments, the antibody or antigen binding fragment thereof comprises a VH comprising the amino acid sequence of SEQ ID NO:113 and a VL comprising the amino acid sequence of SEQ ID NO:144.

In some such embodiments, the antibody or antigen binding fragment thereof comprises a VH comprising the amino acid sequence of SEQ ID NO:121 and a VL comprising the amino acid sequence of SEQ ID NO:144.

In some such embodiments, the antibody or antigen binding fragment thereof comprises a VH comprising the amino acid sequence of SEQ ID NO:118 and a VL comprising the amino acid sequence of SEQ ID NO:144.

In some such embodiments, the antibody or antigen binding fragment thereof comprises a VH comprising the amino acid sequence of SEQ ID NO:122 and a VL comprising the amino acid sequence of SEQ ID NO:144.

In some such embodiments, the antibody or antigen binding fragment thereof comprises a VH comprising the amino acid sequence of SEQ ID NO:123 and a VL comprising the amino acid sequence of SEQ ID NO:144.

In some such embodiments, the antibody or antigen binding fragment thereof comprises a VH comprising the amino acid sequence of SEQ ID NO:113 and a VL comprising the amino acid sequence of SEQ ID NO:146.

In some such embodiments, the antibody or antigen binding fragment thereof comprises a VH comprising the amino acid sequence of SEQ ID NO:121 and a VL comprising the amino acid sequence of SEQ ID NO:146.

In some such embodiments, the antibody or antigen binding fragment thereof comprises a VH comprising the amino acid sequence of SEQ ID NO:118 and a VL comprising the amino acid sequence of SEQ ID NO:146.

In some such embodiments, the antibody or antigen binding fragment thereof comprises a VH comprising the amino acid sequence of SEQ ID NO:122 and a VL comprising the amino acid sequence of SEQ ID NO:146.

In some such embodiments, the antibody or antigen binding fragment thereof comprises a VH comprising the amino acid sequence of SEQ ID NO:123 and a VL comprising the amino acid sequence of SEQ ID NO:146.

In some such embodiments, the antibody or antigen binding fragment thereof comprises a VH comprising the amino acid sequence of SEQ ID NO:113 and a VL comprising the amino acid sequence of SEQ ID NO:145.

In some such embodiments, the antibody or antigen binding fragment thereof comprises a VH comprising the amino acid sequence of SEQ ID NO:121 and a VL comprising the amino acid sequence of SEQ ID NO:145.

In some such embodiments, the antibody or antigen binding fragment thereof comprises a VH comprising the amino acid sequence of SEQ ID NO:118 and a VL comprising the amino acid sequence of SEQ ID NO:145.

In some such embodiments, the antibody or antigen binding fragment thereof comprises a VH comprising the amino acid sequence of SEQ ID NO:122 and a VL comprising the amino acid sequence of SEQ ID NO:145.

In some such embodiments, the antibody or antigen binding fragment thereof comprises a VH comprising the amino acid sequence of SEQ ID NO:123 and a VL comprising the amino acid sequence of SEQ ID NO:145.

In some such embodiments, the antibody or antigen binding fragment thereof comprises a VH comprising the amino acid sequence of SEQ ID NO:114 and a VL comprising the amino acid sequence of SEQ ID NO:146.

In some such embodiments, the antibody or antigen binding fragment thereof comprises a VH comprising the amino acid sequence of SEQ ID NO:115 and a VL comprising the amino acid sequence of SEQ ID NO:146.

In some such embodiments, the antibody or antigen binding fragment thereof comprises a VH comprising the amino acid sequence of SEQ ID NO:116 and a VL comprising the amino acid sequence of SEQ ID NO:146.

In some such embodiments, the antibody or antigen binding fragment thereof comprises a VH comprising the amino acid sequence of SEQ ID NO:117 and a VL comprising the amino acid sequence of SEQ ID NO:146.

In some such embodiments, the antibody or antigen binding fragment thereof comprises a VH comprising the amino acid sequence of SEQ ID NO:124 and a VL comprising the amino acid sequence of SEQ ID NO:146.

In some such embodiments, the antibody or antigen binding fragment thereof comprises a VH comprising the amino acid sequence of SEQ ID NO:125 and a VL comprising the amino acid sequence of SEQ ID NO:146.

In some such embodiments, the antibody or antigen binding fragment thereof comprises a VH comprising the amino acid sequence of SEQ ID NO:119 and a VL comprising the amino acid sequence of SEQ ID NO:146.

In some such embodiments, the antibody or antigen binding fragment thereof comprises a VH comprising the amino acid sequence of SEQ ID NO:120 and a VL comprising the amino acid sequence of SEQ ID NO:146.

In some such embodiments, the antibody or antigen binding fragment thereof comprises a VH comprising the amino acid sequence of SEQ ID NO:113 and a VL comprising the amino acid sequence of SEQ ID NO:147.

In some such embodiments, the antibody or antigen binding fragment thereof comprises a VH comprising the amino acid sequence of SEQ ID NO:118 and a VL comprising the amino acid sequence of SEQ ID NO:147.

In some such embodiments, the antibody or antigen binding fragment thereof comprises a VH comprising the amino acid sequence of SEQ ID NO:114 and a VL comprising the amino acid sequence of SEQ ID NO:147.

In some such embodiments, the antibody or antigen binding fragment thereof comprises a VH comprising the amino acid sequence of SEQ ID NO:115 and a VL comprising the amino acid sequence of SEQ ID NO:147.

In some such embodiments, the antibody or antigen binding fragment thereof comprises a VH comprising the amino acid sequence of SEQ ID NO:116 and a VL comprising the amino acid sequence of SEQ ID NO:147.

In some such embodiments, the antibody or antigen binding fragment thereof comprises a VH comprising the amino acid sequence of SEQ ID NO:117 and a VL comprising the amino acid sequence of SEQ ID NO:147.

In some such embodiments, the antibody or antigen binding fragment thereof comprises a VH comprising the amino acid sequence of SEQ ID NO:124 and a VL comprising the amino acid sequence of SEQ ID NO:147.

In some such embodiments, the antibody or antigen binding fragment thereof comprises a VH comprising the amino acid sequence of SEQ ID NO:125 and a VL comprising the amino acid sequence of SEQ ID NO:147.

In some such embodiments, the antibody or antigen binding fragment thereof comprises a VH comprising the amino acid sequence of SEQ ID NO:119 and a VL comprising the amino acid sequence of SEQ ID NO:147.

In some such embodiments, the antibody or antigen binding fragment thereof comprises a VH comprising the amino acid sequence of SEQ ID NO:120 and a VL comprising the amino acid sequence of SEQ ID NO:147.

In some such embodiments, the antibody or antigen binding fragment thereof comprises a VH comprising the amino acid sequence of SEQ ID NO:113 and a VL comprising the amino acid sequence of SEQ ID NO:148.

In some such embodiments, the antibody or antigen binding fragment thereof comprises a VH comprising the amino acid sequence of SEQ ID NO:118 and a VL comprising the amino acid sequence of SEQ ID NO:148.

In some such embodiments, the antibody or antigen binding fragment thereof comprises a VH comprising the amino acid sequence of SEQ ID NO:114 and a VL comprising the amino acid sequence of SEQ ID NO:148.

In some such embodiments, the antibody or antigen binding fragment thereof comprises a VH comprising the amino acid sequence of SEQ ID NO:115 and a VL comprising the amino acid sequence of SEQ ID NO:148.

In some such embodiments, the antibody or antigen binding fragment thereof comprises a VH comprising the amino acid sequence of SEQ ID NO:116 and a VL comprising the amino acid sequence of SEQ ID NO:148.

In some such embodiments, the antibody or antigen binding fragment thereof comprises a VH comprising the amino acid sequence of SEQ ID NO:117 and a VL comprising the amino acid sequence of SEQ ID NO:148.

In some such embodiments, the antibody or antigen binding fragment thereof comprises a VH comprising the amino acid sequence of SEQ ID NO:124 and a VL comprising the amino acid sequence of SEQ ID NO:148.

In some such embodiments, the antibody or antigen binding fragment thereof comprises a VH comprising the amino acid sequence of SEQ ID NO:125 and a VL comprising the amino acid sequence of SEQ ID NO:148.

In some such embodiments, the antibody or antigen binding fragment thereof comprises a VH comprising the amino acid sequence of SEQ ID NO:119 and a VL comprising the amino acid sequence of SEQ ID NO:148.

In some such embodiments, the antibody or antigen binding fragment thereof comprises a VH comprising the amino acid sequence of SEQ ID NO:120 and a VL comprising the amino acid sequence of SEQ ID NO:148.

In some such embodiments, the antibody or antigen binding fragment thereof comprises a VH comprising the amino acid sequence of SEQ ID NO:113 and a VL comprising the amino acid sequence of SEQ ID NO:149.

In some such embodiments, the antibody or antigen binding fragment thereof comprises a VH comprising the amino acid sequence of SEQ ID NO:118 and a VL comprising the amino acid sequence of SEQ ID NO:149.

In some such embodiments, the antibody or antigen binding fragment thereof comprises a VH comprising the amino acid sequence of SEQ ID NO:114 and a VL comprising the amino acid sequence of SEQ ID NO:149.

In some such embodiments, the antibody or antigen binding fragment thereof comprises a VH comprising the amino acid sequence of SEQ ID NO:115 and a VL comprising the amino acid sequence of SEQ ID NO:149.

In some such embodiments, the antibody or antigen binding fragment thereof comprises a VH comprising the amino acid sequence of SEQ ID NO:116 and a VL comprising the amino acid sequence of SEQ ID NO:149.

In some such embodiments, the antibody or antigen binding fragment thereof comprises a VH comprising the amino acid sequence of SEQ ID NO:117 and a VL comprising the amino acid sequence of SEQ ID NO:149.

In some such embodiments, the antibody or antigen binding fragment thereof comprises a VH comprising the amino acid sequence of SEQ ID NO:124 and a VL comprising the amino acid sequence of SEQ ID NO:149.

In some such embodiments, the antibody or antigen binding fragment thereof comprises a VH comprising the amino acid sequence of SEQ ID NO:125 and a VL comprising the amino acid sequence of SEQ ID NO:149.

In some such embodiments, the antibody or antigen binding fragment thereof comprises a VH comprising the amino acid sequence of SEQ ID NO:119 and a VL comprising the amino acid sequence of SEQ ID NO:149.

In some such embodiments, the antibody or antigen binding fragment thereof comprises a VH comprising the amino acid sequence of SEQ ID NO:120 and a VL comprising the amino acid sequence of SEQ ID NO:149.

In some such embodiments, the antibody or antigen binding fragment thereof comprises a VH comprising the amino acid sequence of SEQ ID NO:114 and a VL comprising the amino acid sequence of SEQ ID NO:143.

In some such embodiments, the antibody or antigen binding fragment thereof comprises a VH comprising the amino acid sequence of SEQ ID NO:114 and a VL comprising the amino acid sequence of SEQ ID NO:144.

In some such embodiments, the antibody or antigen binding fragment thereof comprises a VH comprising the amino acid sequence of SEQ ID NO:114 and a VL comprising the amino acid sequence of SEQ ID NO:145.

In some such embodiments, the antibody or antigen binding fragment thereof comprises a VH comprising the amino acid sequence of SEQ ID NO:115 and a VL comprising the amino acid sequence of SEQ ID NO:143.

In some such embodiments, the antibody or antigen binding fragment thereof comprises a VH comprising the amino acid sequence of SEQ ID NO:115 and a VL comprising the amino acid sequence of SEQ ID NO:144.

In some such embodiments, the antibody or antigen binding fragment thereof comprises a VH comprising the amino acid sequence of SEQ ID NO:115 and a VL comprising the amino acid sequence of SEQ ID NO:145.

In some such embodiments, the antibody or antigen binding fragment thereof comprises a VH comprising the amino acid sequence of SEQ ID NO:116 and a VL comprising the amino acid sequence of SEQ ID NO:143.

In some such embodiments, the antibody or antigen binding fragment thereof comprises a VH comprising the amino acid sequence of SEQ ID NO:116 and a VL comprising the amino acid sequence of SEQ ID NO:144.

In some such embodiments, the antibody or antigen binding fragment thereof comprises a VH comprising the amino acid sequence of SEQ ID NO:116 and a VL comprising the amino acid sequence of SEQ ID NO:145.

In some such embodiments, the antibody or antigen binding fragment thereof comprises a VH comprising the amino acid sequence of SEQ ID NO:117 and a VL comprising the amino acid sequence of SEQ ID NO:143.

In some such embodiments, the antibody or antigen binding fragment thereof comprises a VH comprising the amino acid sequence of SEQ ID NO:117 and a VL comprising the amino acid sequence of SEQ ID NO:144.

In some such embodiments, the antibody or antigen binding fragment thereof comprises a VH comprising the amino acid sequence of SEQ ID NO:117 and a VL comprising the amino acid sequence of SEQ ID NO:145.

In some such embodiments, the antibody or antigen binding fragment thereof comprises a VH comprising the amino acid sequence of SEQ ID NO:119 and a VL comprising the amino acid sequence of SEQ ID NO:143.

In some such embodiments, the antibody or antigen binding fragment thereof comprises a VH comprising the amino acid sequence of SEQ ID NO:119 and a VL comprising the amino acid sequence of SEQ ID NO:144.

In some such embodiments, the antibody or antigen binding fragment thereof comprises a VH comprising the amino acid sequence of SEQ ID NO:119 and a VL comprising the amino acid sequence of SEQ ID NO:145.

In some such embodiments, the antibody or antigen binding fragment thereof comprises a VH comprising the amino acid sequence of SEQ ID NO:120 and a VL comprising the amino acid sequence of SEQ ID NO:143.

In some such embodiments, the antibody or antigen binding fragment thereof comprises a VH comprising the amino acid sequence of SEQ ID NO:120 and a VL comprising the amino acid sequence of SEQ ID NO:144.

In some such embodiments, the antibody or antigen binding fragment thereof comprises a VH comprising the amino acid sequence of SEQ ID NO:120 and a VL comprising the amino acid sequence of SEQ ID NO:145.

In some such embodiments, the antibody or antigen binding fragment thereof comprises a VH comprising the amino acid sequence of SEQ ID NO:121 and a VL comprising the amino acid sequence of SEQ ID NO:147.

In some such embodiments, the antibody or antigen binding fragment thereof comprises a VH comprising the amino acid sequence of SEQ ID NO:121 and a VL comprising the amino acid sequence of SEQ ID NO:148.

In some such embodiments, the antibody or antigen binding fragment thereof comprises a VH comprising the amino acid sequence of SEQ ID NO:121 and a VL comprising the amino acid sequence of SEQ ID NO:149.

In some such embodiments, the antibody or antigen binding fragment thereof comprises a VH comprising the amino acid sequence of SEQ ID NO:122 and a VL comprising the amino acid sequence of SEQ ID NO:147.

In some such embodiments, the antibody or antigen binding fragment thereof comprises a VH comprising the amino acid sequence of SEQ ID NO:122 and a VL comprising the amino acid sequence of SEQ ID NO:148.

In some such embodiments, the antibody or antigen binding fragment thereof comprises a VH comprising the amino acid sequence of SEQ ID NO:122 and a VL comprising the amino acid sequence of SEQ ID NO:149.

In some such embodiments, the antibody or antigen binding fragment thereof comprises a VH comprising the amino acid sequence of SEQ ID NO:123 and a VL comprising the amino acid sequence of SEQ ID NO:147.

In some such embodiments, the antibody or antigen binding fragment thereof comprises a VH comprising the amino acid sequence of SEQ ID NO:123 and a VL comprising the amino acid sequence of SEQ ID NO:148.

In some such embodiments, the antibody or antigen binding fragment thereof comprises a VH comprising the amino acid sequence of SEQ ID NO:123 and a VL comprising the amino acid sequence of SEQ ID NO:149.

In some such embodiments, the antibody or antigen binding fragment thereof comprises a VH comprising the amino acid sequence of SEQ ID NO:124 and a VL comprising the amino acid sequence of SEQ ID NO:143.

In some such embodiments, the antibody or antigen binding fragment thereof comprises a VH comprising the amino acid sequence of SEQ ID NO:124 and a VL comprising the amino acid sequence of SEQ ID NO:144.

In some such embodiments, the antibody or antigen binding fragment thereof comprises a VH comprising the amino acid sequence of SEQ ID NO:124 and a VL comprising the amino acid sequence of SEQ ID NO:145.

In some such embodiments, the antibody or antigen binding fragment thereof comprises a VH comprising the amino acid sequence of SEQ ID NO:125 and a VL comprising the amino acid sequence of SEQ ID NO:143.

In some such embodiments, the antibody or antigen binding fragment thereof comprises a VH comprising the amino acid sequence of SEQ ID NO:125 and a VL comprising the amino acid sequence of SEQ ID NO:144.

In some such embodiments, the antibody or antigen binding fragment thereof comprises a VH comprising the amino acid sequence of SEQ ID NO:125 and a VL comprising the amino acid sequence of SEQ ID NO:145.

In some such embodiments, the antibody or antigen binding fragment thereof comprises a VH comprising the amino acid sequence of SEQ ID NO:40, and a VL comprising the amino acid sequence of SEQ ID NO:128.

In some such embodiments, the antibody or antigen binding fragment thereof comprises a VH comprising the amino acid sequence selected from any one of SEQ ID NOS:94-102, and a VL comprising the amino acid sequence selected from any one of SEQ ID NOS:138-141.

In some such embodiments, the antibody or antigen binding fragment thereof comprises a VH comprising the amino acid sequence of SEQ ID NO:94 and a VL comprising the amino acid sequence of SEQ ID NO:138.

In some such embodiments, the antibody or antigen binding fragment thereof comprises a VH comprising the amino acid sequence of SEQ ID NO:95 and a VL comprising the amino acid sequence of SEQ ID NO:138.

In some such embodiments, the antibody or antigen binding fragment thereof comprises a VH comprising the amino acid sequence of SEQ ID NO:96 and a VL comprising the amino acid sequence of SEQ ID NO:138.

In some such embodiments, the antibody or antigen binding fragment thereof comprises a VH comprising the amino acid sequence of SEQ ID NO:97 and a VL comprising the amino acid sequence of SEQ ID NO:138.

In some such embodiments, the antibody or antigen binding fragment thereof comprises a VH comprising the amino acid sequence of SEQ ID NO:98 and a VL comprising the amino acid sequence of SEQ ID NO:138.

In some such embodiments, the antibody or antigen binding fragment thereof comprises a VH comprising the amino acid sequence of SEQ ID NO:99 and a VL comprising the amino acid sequence of SEQ ID NO:138.

In some such embodiments, the antibody or antigen binding fragment thereof comprises a VH comprising the amino acid sequence of SEQ ID NO:100 and a VL comprising the amino acid sequence of SEQ ID NO:138.

In some such embodiments, the antibody or antigen binding fragment thereof comprises a VH comprising the amino acid sequence of SEQ ID NO:101 and a VL comprising the amino acid sequence of SEQ ID NO:138.

In some such embodiments, the antibody or antigen binding fragment thereof comprises a VH comprising the amino acid sequence of SEQ ID NO:102 and a VL comprising the amino acid sequence of SEQ ID NO:138.

In some such embodiments, the antibody or antigen binding fragment thereof comprises a VH comprising the amino acid sequence of SEQ ID NO:96 and a VL comprising the amino acid sequence of SEQ ID NO:139.

In some such embodiments, the antibody or antigen binding fragment thereof comprises a VH comprising the amino acid sequence of SEQ ID NO:96 and a VL comprising the amino acid sequence of SEQ ID NO:140.

In some such embodiments, the antibody or antigen binding fragment thereof comprises a VH comprising the amino acid sequence of SEQ ID NO:96 and a VL comprising the amino acid sequence of SEQ ID NO:141.

In some such embodiments, the antibody or antigen binding fragment thereof comprises a VH comprising the amino acid sequence of SEQ ID NO:98 and a VL comprising the amino acid sequence of SEQ ID NO:140.

In some such embodiments, the antibody or antigen binding fragment thereof comprises a VH comprising the amino acid sequence of SEQ ID NO:100 and a VL comprising the amino acid sequence of SEQ ID NO:140.

In some such embodiments, the antibody or antigen binding fragment thereof comprises a VH comprising the amino acid sequence of SEQ ID NO:94 and a VL comprising the amino acid sequence of SEQ ID NO:139.

In some such embodiments, the antibody or antigen binding fragment thereof comprises a VH comprising the amino acid sequence of SEQ ID NO:94 and a VL comprising the amino acid sequence of SEQ ID NO:140.

In some such embodiments, the antibody or antigen binding fragment thereof comprises a VH comprising the amino acid sequence of SEQ ID NO:94 and a VL comprising the amino acid sequence of SEQ ID NO:141.

In some such embodiments, the antibody or antigen binding fragment thereof comprises a VH comprising the amino acid sequence of SEQ ID NO:95 and a VL comprising the amino acid sequence of SEQ ID NO:139.

In some such embodiments, the antibody or antigen binding fragment thereof comprises a VH comprising the amino acid sequence of SEQ ID NO:95 and a VL comprising the amino acid sequence of SEQ ID NO:140.

In some such embodiments, the antibody or antigen binding fragment thereof comprises a VH comprising the amino acid sequence of SEQ ID NO:95 and a VL comprising the amino acid sequence of SEQ ID NO:141.

In some such embodiments, the antibody or antigen binding fragment thereof comprises a VH comprising the amino acid sequence of SEQ ID NO:97 and a VL comprising the amino acid sequence of SEQ ID NO:138.

In some such embodiments, the antibody or antigen binding fragment thereof comprises a VH comprising the amino acid sequence of SEQ ID NO:97 and a VL comprising the amino acid sequence of SEQ ID NO:139.

In some such embodiments, the antibody or antigen binding fragment thereof comprises a VH comprising the amino acid sequence of SEQ ID NO:97 and a VL comprising the amino acid sequence of SEQ ID NO:140.

In some such embodiments, the antibody or antigen binding fragment thereof comprises a VH comprising the amino acid sequence of SEQ ID NO:97 and a VL comprising the amino acid sequence of SEQ ID NO:141.

In some such embodiments, the antibody or antigen binding fragment thereof comprises a VH comprising the amino acid sequence of SEQ ID NO:98 and a VL comprising the amino acid sequence of SEQ ID NO:138.

In some such embodiments, the antibody or antigen binding fragment thereof comprises a VH comprising the amino acid sequence of SEQ ID NO:98 and a VL comprising the amino acid sequence of SEQ ID NO:139.

In some such embodiments, the antibody or antigen binding fragment thereof comprises a VH comprising the amino acid sequence of SEQ ID NO:98 and a VL comprising the amino acid sequence of SEQ ID NO:141.

In some such embodiments, the antibody or antigen binding fragment thereof comprises a VH comprising the amino acid sequence of SEQ ID NO:99 and a VL comprising the amino acid sequence of SEQ ID NO:138.

In some such embodiments, the antibody or antigen binding fragment thereof comprises a VH comprising the amino acid sequence of SEQ ID NO:99 and a VL comprising the amino acid sequence of SEQ ID NO:139.

In some such embodiments, the antibody or antigen binding fragment thereof comprises a VH comprising the amino acid sequence of SEQ ID NO:99 and a VL comprising the amino acid sequence of SEQ ID NO:140.

In some such embodiments, the antibody or antigen binding fragment thereof comprises a VH comprising the amino acid sequence of SEQ ID NO:99 and a VL comprising the amino acid sequence of SEQ ID NO:141.

In some such embodiments, the antibody or antigen binding fragment thereof comprises a VH comprising the amino acid sequence of SEQ ID NO:100 and a VL comprising the amino acid sequence of SEQ ID NO:138.

In some such embodiments, the antibody or antigen binding fragment thereof comprises a VH comprising the amino acid sequence of SEQ ID NO:100 and a VL comprising the amino acid sequence of SEQ ID NO:139.

In some such embodiments, the antibody or antigen binding fragment thereof comprises a VH comprising the amino acid sequence of SEQ ID NO:100 and a VL comprising the amino acid sequence of SEQ ID NO:141.

In some such embodiments, the antibody or antigen binding fragment thereof comprises a VH comprising the amino acid sequence of SEQ ID NO:101 and a VL comprising the amino acid sequence of SEQ ID NO:138.

In some such embodiments, the antibody or antigen binding fragment thereof comprises a VH comprising the amino acid sequence of SEQ ID NO:101 and a VL comprising the amino acid sequence of SEQ ID NO:139.

In some such embodiments, the antibody or antigen binding fragment thereof comprises a VH comprising the amino acid sequence of SEQ ID NO:101 and a VL comprising the amino acid sequence of SEQ ID NO:140.

In some such embodiments, the antibody or antigen binding fragment thereof comprises a VH comprising the amino acid sequence of SEQ ID NO:101 and a VL comprising the amino acid sequence of SEQ ID NO:141.

In some such embodiments, the antibody or antigen binding fragment thereof comprises a VH comprising the amino acid sequence of SEQ ID NO:102 and a VL comprising the amino acid sequence of SEQ ID NO:138.

In some such embodiments, the antibody or antigen binding fragment thereof comprises a VH comprising the amino acid sequence of SEQ ID NO:102 and a VL comprising the amino acid sequence of SEQ ID NO:139.

In some such embodiments, the antibody or antigen binding fragment thereof comprises a VH comprising the amino acid sequence of SEQ ID NO:102 and a VL comprising the amino acid sequence of SEQ ID NO:140.

In some such embodiments, the antibody or antigen binding fragment thereof comprises a VH comprising the amino acid sequence of SEQ ID NO:102 and a VL comprising the amino acid sequence of SEQ ID NO:141.

In some such embodiments, the antibody or antigen binding fragment thereof comprises a VH comprising the amino acid sequence of SEQ ID NO:41, and a VL comprising the amino acid sequence of SEQ ID NO:129.

In some such embodiments, the antibody or antigen binding fragment thereof comprises a VH comprising the amino acid sequence selected from any one of SEQ ID NOS:103-112, and a VL comprising the amino acid sequence of SEQ ID NO:142.

In some such embodiments, the antibody or antigen binding fragment thereof comprises a VH comprising the amino acid sequence of SEQ ID NO:103 and a VL comprising the amino acid sequence of SEQ ID NO:142.

In some such embodiments, the antibody or antigen binding fragment thereof comprises a VH comprising the amino acid sequence of SEQ ID NO:104 and a VL comprising the amino acid sequence of SEQ ID NO:142.

In some such embodiments, the antibody or antigen binding fragment thereof comprises a VH comprising the amino acid sequence of SEQ ID NO:105 and a VL comprising the amino acid sequence of SEQ ID NO:142.

In some such embodiments, the antibody or antigen binding fragment thereof comprises a VH comprising the amino acid sequence of SEQ ID NO:106 and a VL comprising the amino acid sequence of SEQ ID NO:142.

In some such embodiments, the antibody or antigen binding fragment thereof comprises a VH comprising the amino acid sequence of SEQ ID NO:107 and a VL comprising the amino acid sequence of SEQ ID NO:142.

In some such embodiments, the antibody or antigen binding fragment thereof comprises a VH comprising the amino acid sequence of SEQ ID NO:108 and a VL comprising the amino acid sequence of SEQ ID NO:142.

In some such embodiments, the antibody or antigen binding fragment thereof comprises a VH comprising the amino acid sequence of SEQ ID NO:109 and a VL comprising the amino acid sequence of SEQ ID NO:142.

In some such embodiments, the antibody or antigen binding fragment thereof comprises a VH comprising the amino acid sequence of SEQ ID NO:110 and a VL comprising the amino acid sequence of SEQ ID NO:142.

In some such embodiments, the antibody or antigen binding fragment thereof comprises a VH comprising the amino acid sequence of SEQ ID NO:111 and a VL comprising the amino acid sequence of SEQ ID NO:142.

In some such embodiments, the antibody or antigen binding fragment thereof comprises a VH comprising the amino acid sequence of SEQ ID NO:112 and a VL comprising the amino acid sequence of SEQ ID NO:142.

In any of the aforementioned embodiments, the VH may be joined to a heavy chain constant region, such as a human IgG1 constant region of SEQ ID NO:230 or 231.

In any of the aforementioned embodiments, the VL may be joined to a light chain constant region, such as a human kappa constant region of SEQ ID NO:232.

In some embodiments, the antibody or antigen binding fragment thereof comprises a heavy chain comprising an amino acid sequence that is at least 90%, 91%, 92%, 93%, 94%, 95%, 96%, 97%, 98%, or 99% identical to the amino acid sequence of SEQ ID NO:150, and a light chain comprising an amino acid sequence that is at least 90%, 91%, 92%, 93%, 94%, 95%, 96%, 97%, 98%, or 99% identical to the amino acid sequence of SEQ ID NO:206, provided that the amino acid sequences of the VH-CDRs and VL-CDRs are unchanged.

In some embodiments, the antibody or antigen binding fragment thereof comprises a heavy chain comprising an amino acid sequence that is at least 90%, 91%, 92%, 93%, 94%, 95%, 96%, 97%, 98%, or 99% identical to the amino acid sequence selected from any one of SEQ ID NOS:155-200, and a light chain comprising an amino acid sequence that is at least 90%, 91%, 92%, 93%, 94%, 95%, 96%, 97%, 98%, or 99% identical to the amino acid sequence selected from any one of SEQ ID NOS:211-213, provided that the amino acid sequences of the VH-CDRs and VL-CDRs are unchanged.

In some embodiments, the antibody or antigen binding fragment thereof comprises a heavy chain comprising an amino acid sequence that is at least 90%, 91%, 92%, 93%, 94%, 95%, 96%, 97%, 98%, or 99% identical to the amino acid sequence of SEQ ID NO:151, and a light chain comprising an amino acid sequence that is at least 90%, 91%, 92%, 93%, 94%, 95%, 96%, 97%, 98%, or 99% identical to the amino acid sequence of SEQ ID NO:207, provided that the amino acid sequences of the VH-CDRs and VL-CDRs are unchanged.

In some embodiments, the antibody or antigen binding fragment thereof comprises a heavy chain comprising an amino acid sequence that is at least 90%, 91%, 92%, 93%, 94%, 95%, 96%, 97%, 98%, or 99% identical to the amino acid sequence selected from any one of SEQ ID NOS:201-205, and a light chain comprising an amino acid sequence that is at least 90%, 91%, 92%, 93%, 94%, 95%, 96%, 97%, 98%, or 99% identical to the amino acid sequence selected from any one of SEQ ID NOS:214-217, provided that the amino acid sequences of the VH-CDRs and VL-CDRs are unchanged.

In some embodiments, the antibody or antigen binding fragment thereof comprises a heavy chain comprising an amino acid sequence that is at least 90%, 91%, 92%, 93%, 94%, 95%, 96%, 97%, 98%, or 99% identical to the amino acid sequence of SEQ ID NO:154, and a light chain comprising an amino acid sequence that is at least 90%, 91%, 92%, 93%, 94%, 95%, 96%, 97%, 98%, or 99% identical to the amino acid sequence of SEQ ID NO:210, provided that the amino acid sequences of the VH-CDRs and VL-CDRs are unchanged.

In some embodiments, the antibody or antigen binding fragment thereof comprises a heavy chain comprising an amino acid sequence that is at least 90%, 91%, 92%, 93%, 94%, 95%, 96%, 97%, 98%, or 99% identical to the amino acid sequence of SEQ ID NO:152, and a light chain comprising an amino acid sequence that is at least 90%, 91%, 92%, 93%, 94%, 95%, 96%, 97%, 98%, or 99% identical to the amino acid sequence of SEQ ID NO:208, provided that the amino acid sequences of the VH-CDRs and VL-CDRs are unchanged.

In some embodiments, the antibody or antigen binding fragment thereof comprises a heavy chain comprising an amino acid sequence that is at least 90%, 91%, 92%, 93%, 94%, 95%, 96%, 97%, 98%, or 99% identical to the amino acid sequence of SEQ ID NO:153, and a light chain comprising an amino acid sequence that is at least 90%, 91%, 92%, 93%, 94%, 95%, 96%, 97%, 98%, or 99% identical to the amino acid sequence of SEQ ID NO:209, provided that the amino acid sequences of the VH-CDRs and VL-CDRs are unchanged.

In some embodiments, the antibody or antigen binding fragment thereof comprises a heavy chain comprising the amino acid sequence of SEQ ID NO:150, and a light chain comprising the amino acid sequence of SEQ ID NO:206.

In some embodiments, the antibody or antigen binding fragment thereof comprises a heavy chain comprising the amino acid sequence selected from any one of SEQ ID NOS:155-200, and a light chain comprising the amino acid sequence selected from any one of SEQ ID NOS:211-213.

In some embodiments, the antibody or antigen binding fragment thereof comprises a heavy chain comprising the amino acid sequence of SEQ ID NO:151, and a light chain comprising the amino acid sequence of SEQ ID NO:207.

In some embodiments, the antibody or antigen binding fragment thereof comprises a heavy chain comprising the amino acid sequence selected from any one of SEQ ID NOS:201-205, and a light chain comprising the amino acid sequence selected from any one of SEQ ID NOS:214-217.

In some embodiments, the antibody or antigen binding fragment thereof comprises a heavy chain comprising the amino acid sequence of SEQ ID NO:154, and a light chain comprising the amino acid sequence of SEQ ID NO:210.

In some embodiments, the antibody or antigen binding fragment thereof comprises a heavy chain comprising the amino acid sequence of SEQ ID NO:152, and a light chain comprising the amino acid sequence of SEQ ID NO:208.

In some embodiments, the antibody or antigen binding fragment thereof comprises a heavy chain comprising the amino acid sequence of SEQ ID NO:153, and a light chain comprising the amino acid sequence of SEQ ID NO:209.

### TGFβR1 Inhibitors

The antibody constructs specific for ASGR1 or ASGR2 as described herein are attached to a TGFβR1 inhibitor to form a conjugate. The TGFβR1 inhibitor can provide a direct or indirect effect. In certain embodiments, the TGFβR1 inhibitor can be coupled to the antibody construct, such as to the Fc domain of the antibody construct.

In some embodiments, a TGFβR1 inhibitor used in a conjugate of this disclosure is not a siRNA, antisense RNA, or other oligonucleotide. In some embodiments, a TGFβR1 inhibitor used in a conjugate of this disclosure is a non-naturally occurring small molecule. A “small molecule” is an organic compound with a molecular weight of less than 1500, or 1000, or 900, or 750, or 600, or 500 Daltons. In some embodiments, a small molecule TGFβR1 inhibitor has an octanol-water partition coefficient (log P) in the range of 3 to 6, or from 4 to 5, or from 2 to 4. In some embodiments, a small molecule TGFβR1 inhibitor has a polar surface area of less than 200, or less than 150 Å². In some embodiments, a small molecule TGFβR1 inhibitor has not more than five, or not more than three, hydrogen bond donors, and not more than 10, or not more than three hydrogen bond acceptors. A small molecule TGFβR1 inhibitor is not a protein, a polysaccharide, or a nucleic acid.

TGFβR1 Inhibitors of Formula (A-I)

In some aspects, the TGFβR1 inhibitor is a compound of Formula (A-I):

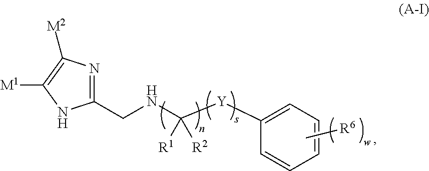

wherein
one of M¹ and M² is

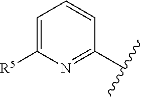

and the other of M¹ and M² is selected from:

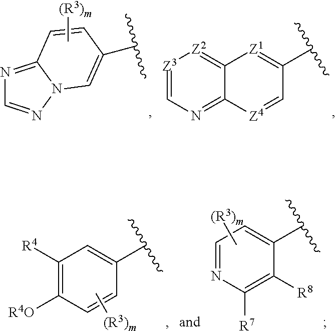

R¹ and R² are, at each occurrence, independently selected from hydrogen, halogen, —OR¹¹, —SR¹¹, —N(R¹¹)₂, —NO₂, —CN, phenyl, and —C₁-C₆ alkyl, wherein said —C₁-C₆ alkyl is optionally substituted with one or more substituents independently selected from halogen, —OR¹¹, —SR¹¹, —S(O)R¹⁰, —S(O)₂R¹¹, —S(O)₂N(R¹¹)₂—N(R¹¹)₂, —C(O)R¹⁰, —C(O)N(R¹¹)₂, —N(R¹¹)C(O)R¹⁰, —C(O)OR¹¹, —OC(O)R¹⁰, —NO₂, and —CN;

R³ is, at each occurrence, independently selected from halogen, —C₁-C₃ alkyl, —C₁-C₃ haloalkyl, —OH, —NO₂, —CN, —OC₁-C₃ alkyl, and —OC₁-C₃ haloalkyl;

each R⁴ is, at each occurrence, independently selected from hydrogen and C₁-C₃ alkyl or two R⁴ join together with atoms to which they are attached to form a 5- or 6-membered heterocycle optionally substituted with one or more substituents independently selected from halogen, C₁-C₃ alkyl, —OH, OC₁-C₃ alkyl, and —OC₁-C₃ haloalkyl;

R⁵ is hydrogen, halogen, —OR⁶¹, —SR⁶¹, —N(R⁶¹)₂, —NO₂, —CN, and —C₁-C₆ alkyl, wherein said —C₁-C₆ alkyl is optionally substituted with one or more substituents independently selected from halogen, —OR⁶¹, —SR⁶¹, —N(R⁶¹)₂, —NO₂, and —CN;

R⁶ is, at each occurrence, independently selected from:
halogen, —OR²¹, —SR²¹, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)₂, —NO₂, and —CN;
C₁-C₁₀ alkyl, C₂-C₁₀ alkenyl, and C₂-C₁₀ alkynyl, each of which is optionally substituted with one or more substituents independently selected from halogen,
   —OR²¹, —SR²¹, —N(R²¹)₂, —C(O)R²⁰, —C(O)N(R²¹)₂, —N(R²¹)C(O)R²⁰, —C(O)OR²¹, —OC(O)R²¹,
   —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)₂, —NO₂, ═O, ═S, ═N(R²¹), —CN, a C₃-C₁₀ carbocycle, and a 3- to 10-membered heterocycle wherein said C₃-C₁₀ carbocycle and said 3- to 10-membered heterocycle are optionally substituted with one or more substituents independently selected from R^{X}; and
a C₃-C₁₀ carbocycle and a 3- to 10-membered heterocycle, each of which is optionally substituted with one or more substituents independently selected from halogen, —OR²⁰, —OH, —SR²⁰, —SH, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)₂, —NO₂, ═O, ═S, ═N(R²¹), —CN, —C₂-C₆ alkenyl,
   —C₂-C₆ alkynyl and C₁-C₆ alkyl wherein said C₁-C₆ alkyl is optionally substituted with one or more substituents independently selected from R^{Y};

R⁷ and R⁸ are independently selected from hydrogen, halogen, C₁-C₃ alkyl, —OH, OC₁-C₃ alkyl, and —OC₁-C₃ haloalkyl, or R⁷ and R⁸ join together with the atoms to which they are attached to form a C₅-C₆ carbocycle or 5- or 6-membered heterocycle each of which is optionally substituted with one or more substituents independently selected from halogen, —OR³¹, —SR³¹, —N(R³¹)₂, —NO₂, —CN and —C₁-C₆ alkyl wherein said C₁-C₆ alkyl is optionally substituted with one or more substituents independently selected from halogen, —OR³¹, —SR³¹, —N(R³¹)₂, —NO₂, and —CN;

Y is selected from —O— and —N(R⁹)— and R⁹ is, at each occurrence, independently selected from: hydrogen; and —C₁-C₆ alkyl optionally substituted with one or more substituents independently selected from halogen, —OR⁴¹, —SR⁴¹, —S(O)R⁴⁰, —S(O)₂R⁴¹, —S(O)₂N(R⁴¹)₂, —N(R⁴¹)₂, —C(O)R⁴⁰, —C(O)N(R⁴¹)₂, —N(R⁴¹)C(O)R⁴⁰, —C(O)OR⁴¹, —OC(O)R⁴⁰, —NO₂, and —CN;

each R¹⁰, R²⁰, and R⁴⁰ is independently selected at each occurrence from:
—C₁-C₁₀ alkyl, —C₂-C₁₀ alkenyl, and —C₂-C₁₀ alkynyl, each of which is optionally substituted with one or more substituents independently selected from R^{Y}; and
a C₃-C₁₂ carbocycle and a 3- to 12-membered heterocycle, each of which is optionally substituted with one or more substituents independently selected from R^{X};

each R¹¹, R²¹, R³¹, R⁴¹, and R⁶¹ is independently selected at each occurrence from:
hydrogen;
—C₁-C₁₀ alkyl, —C₂-C₁₀ alkenyl, and —C₂-C₁₀ alkynyl, each of which is optionally substituted with one or more substituents independently selected from R^{Y}; and
a C₃-C₁₂ carbocycle and a 3- to 12-membered heterocycle, each of which is optionally substituted with one or more substituents independently selected from R^{X},
or two R¹¹, R²¹, R³¹, R⁴¹, or R⁶¹ on the same N atom are taken together with the N atom to which they are attached to form a N-containing heterocycle optionally substituted with R^{X};

each R^{X} is independently selected at each occurrence from: halogen, —OR⁵¹, —SR⁵¹, —N(R⁵¹)₂, —C(O)R⁵⁰, —C(O)N(R⁵¹)₂, —N(R⁵¹)C(O)R⁵⁰, —C(O)OR⁵¹, —OC(O)R⁵¹, —S(O)R⁵⁰, —S(O)₂R⁵¹, —S(O)₂N(R⁵¹)₂, —OC(O)OR⁵¹, —OC(O)N(R⁵¹)₂, —NR¹⁵C(═O)OR⁵¹, —N(R⁵¹)C(O)N(R⁵¹)₂, —NO₂, ═O, ═S, ═N(R⁵¹), —CN, —C₂-C₆ alkenyl, —C₂-C₆ alkynyl, and C₁-C₆ alkyl, wherein said C₁-C₆ alkyl is optionally substituted with one or more substituents independently selected from —OR⁵¹, —SR⁵¹, —N(R⁵¹)₂, —C(O)R⁵⁰—C(O)N(R⁵¹)₂,

—N(R⁵¹)C(O)R⁵⁰, —C(O)OR⁵¹, —OC(O)R⁵¹, —S(O)R⁵⁰, —S(O)₂R⁵¹, —S(O)₂N(R⁵¹)₂, —OC(O)OR⁵¹, —OC(O)N(R⁵¹)₂—NR⁵¹C(═O)OR⁵¹, —N(R⁵¹)C(O)N(R⁵¹)₂, and ═O;

each R^{Y} is independently selected at each occurrence from: halogen, —OR⁵¹, —SR⁵¹, —N(R⁵¹)₂, —C(O)R⁵⁰, —C(O)N(R⁵¹)₂, —N(R⁵¹)C(O)R⁵⁰, —C(O)OR⁵¹, —OC(O)R⁵¹, —S(O)R⁵⁰, —S(O)₂R⁵¹, —S(O)₂N(R⁵¹)₂, —OC(O)OR⁵¹, —OC(O)N(R⁵¹)₂, —NR⁵¹C(═O)OR⁵¹, —N(R⁵¹)C(O)N(R⁵¹)₂, —NO₂, ═O, ═S, ═N(R⁵¹), and —CN;
each R⁵⁰ is independently selected at each occurrence from:
—C₁-C₁₀ alkyl, —C₂-C₁₀ alkenyl, and —C₂-C₁₀ alkynyl, each of which is optionally substituted with one or more substituents independently selected from halogen,
   —OH, —CN, —NO₂, —NH₂, ═O, ═S, —O—C₁-C₁₀ alkyl, C₃-C₁₂ carbocycle, and a 3- to 12-membered heterocycle; and
a C₃-C₁₂ carbocycle and a 3- to 12-membered heterocycle, each of which is optionally substituted with one or more substituents independently selected from halogen, —OH, —CN, —NO₂, —NH₂, ═O, ═S, —C₁-C₁₀ alkyl, —O—C₁-C₁₀ alkyl, and —C₁-C₁₀ haloalkyl;

each R⁵¹ is independently selected at each occurrence from:
hydrogen;
—C₁-C₁₀ alkyl, —C₂-C₁₀ alkenyl, and —C₂-C₁₀ alkynyl, each of which is optionally substituted with one or more substituents independently selected from halogen,
   —OH, —CN, —NO₂, —NH₂, ═O, ═S, —O—C₁-C₁₀ alkyl, C₃-C₁₂ carbocycle, and a 3- to 12-membered heterocycle; and
a C₃-C₁₂ carbocycle and a 3- to 12-membered heterocycle, each of which is optionally substituted with one or more substituents independently selected from halogen, —OH, —CN, —NO₂, —NH₂, ═O, ═S, —C₁-C₁₀ alkyl, —O—C₁-C₁₀ alkyl, and —C₁-C₁₀ haloalkyl;

Z¹, Z², Z³, and Z⁴ are each independently selected from N or C(H);

n is selected from 1, 2, and 3;

m is 0, 1, or 2;

s is selected from 0 and 1; and

w is selected from 0, 1, 2, 3, 4, and 5,

or a salt thereof.

In a second aspect, disclosed herein is a compound represented by Formula (A-I) or a salt thereof, wherein one of M¹ and M² is

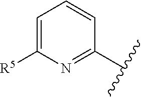

and the other of M¹ and M² is

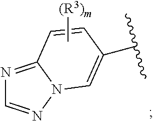

and the remaining variables (e.g., R¹-R⁸, R¹⁰, R²⁰, R⁴⁰, R¹¹, R²¹, R³¹, R⁴¹, R⁵⁰, R⁵¹, R⁶¹, Y, R^{X}, R^{Y}, Z¹, Z², Z³, Z⁴, n, m, s, and w) are as set forth in the first aspect.

In a third aspect, disclosed herein is a compound represented by Formula (A-I) or a salt thereof, wherein one of M¹ and M² is

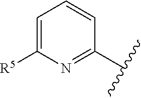

and the other of M¹ and M² is

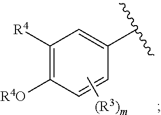

and the remaining variables are as set forth in the first aspect.

In a fourth aspect, disclosed herein is a compound represented by Formula (A-I) wherein one of one of M¹ and M² is

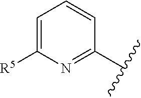

and the other of M¹ and M² is

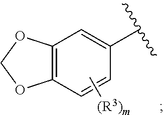

and the remaining variables are as set forth in the first aspect.

In a fifth aspect, disclosed herein is a compound represented by Formula (A-I) or a salt thereof, wherein one of one of M¹ and M² is

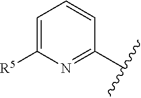

and the other of M¹ and M² is

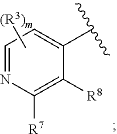

and the remaining variables are as set forth in the first aspect.

In a sixth aspect, disclosed herein is a compound represented by Formula (A-I) or a salt thereof, wherein M¹ and M² is as set forth in the fifth aspect and R⁷ and R are independently selected from hydrogen, halogen, C₁-C₃ alkyl, —OH, OC₁-C₃ alkyl, and —OC₁-C₃ haloalkyl, or R⁷ and R⁸ join together with the atoms to which they are attached to form a C₅-C₆ carbocycle or 5- or 6-membered heterocycle each of which is optionally substituted with one or more substituents independently selected from halogen, —OR³¹,

—SR³¹, —N(R³¹)₂, and —C₁-C₆ alkyl wherein said C₁-C₆ alkyl is optionally substituted with one or more substituents independently selected from halogen, —OR³¹, —SR³¹, and —N(R³¹)₂; and the remaining variables are as set forth in the first aspect.

In a seventh aspect, disclosed herein is a compound represented by Formula (A-I) or a salt thereof, wherein M¹ and M² is as set forth in the fifth aspect and R⁷ and R⁸ are independently selected from hydrogen, halogen, C₁-C₃ alkyl, —OH, OC₁-C₃ alkyl, and —OC₁-C₃ haloalkyl, or R⁷ and R⁸ join together with the atoms to which they are attached to form an unsubstituted C₅-C₆ carbocycle or an unsubstituted 5- or 6-membered heterocycle; and the remaining variables are as set forth in the first aspect.

In an eighth aspect, disclosed herein is a compound represented by Formula (A-I) or a salt thereof, wherein M¹ and M² is as set forth in any one of aspects 5-7 wherein the 5- or 6-membered heterocycle of R⁷ and R⁸ is a 5- or 6-membered heterocycle contains one ring heteroatom selected from nitrogen contains one ring heteroatom selected from nitrogen; and the remaining variables are as set forth in the first aspect.

In a ninth aspect, disclosed herein is a compound represented by Formula (A-I) or a salt thereof, wherein M¹ and M² is as set forth in any one of aspects 5-7 wherein R⁷ and R⁸ join together with the atoms to which they are attached to form a phenyl ring optionally substituted with one or more substituents independently selected from halogen,

—OR³¹, —SR³¹, —N(R³¹)₂, and —C₁-C₆ alkyl wherein said C₁-C₆ alkyl is optionally substituted with one or more substituents independently selected from halogen, —OR³¹, —SR³¹, and
—N(R³¹)₂; and the remaining variables are as set forth in the first aspect.

In a tenth aspect, disclosed herein is a compound represented by Formula (A-I) or a salt thereof, wherein M¹ and M² is as set forth in any one of aspects 5-7 wherein R⁷ and R⁸ join together with the atoms to which they are attached to form an unsubstituted phenyl ring; and the remaining variables are as set forth in the first aspect.

In an eleventh aspect, disclosed herein is a compound represented by Formula (A-I) or a salt thereof, wherein M¹ and M² is as set forth in any one of aspects 5-7 wherein R⁷ and R⁸ are each hydrogen; and the remaining variables are as set forth in the first aspect.

In a twelfth aspect, disclosed herein is a compound represented by Formula (A-I) or a salt thereof, wherein M¹, M², R⁷ and R⁸ are as set forth in any one of aspects 1-11 and wherein m is 1 or 2 and R³ is, at each occurrence, independently selected from halogen, —C₁-C₃ alkyl, —C₁-C₃ haloalkyl, —OH, OC₁-C₃ alkyl, and —OC₁-C₃ haloalkyl; and the remaining variables are as set forth in the first aspect.

In a thirteenth aspect, disclosed herein is a compound represented by Formula (A-I) or a salt thereof, wherein M¹, M², R⁷ and R⁸ are as set forth in any one of aspects 1-11 and wherein m is 1 and R³ is, at each occurrence, independently selected from halogen, —C₁-C₃ alkyl, —C₁-C₃ haloalkyl, —OH, and —OC₁-C₃ alkyl; and the remaining variables are as set forth in the first aspect.

In a fourteenth aspect, disclosed herein is a compound represented by Formula (A-I) or a salt thereof, wherein M¹, M², R⁷ and R⁸ are as set forth in any one of aspects 1-11 and wherein m is zero; and the remaining variables are as set forth in the first aspect.

In a fifteenth aspect, disclosed herein is a compound represented by Formula (A-I) or a salt thereof, wherein one of one of M¹ and M² is

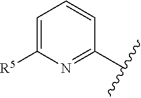

and the other of M¹ and M² is

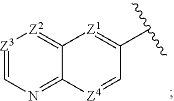

and the remaining variables are as set forth in the first aspect.

In a sixteenth aspect, disclosed herein is a compound represented by Formula (A-I) or a salt thereof, wherein M¹ and M² is as set forth in aspect 15 wherein Z¹, Z², Z³, and Z⁴ are —C(H); and the remaining variables are as set forth in the first aspect.

In a seventeenth aspect, disclosed herein is a compound represented by Formula (A-I) or a salt thereof, wherein M¹ and M² is as set forth in aspect 15 wherein Z² is N and Z¹, Z³, and Z⁴ are —C(H); and the remaining variables are as set forth in the first aspect.

In a eighteenth aspect, disclosed herein is a compound represented by Formula (A-I) or a salt thereof, wherein M¹ and M² are as set forth in aspect 15 wherein Z¹ is N and Z², Z³, and Z⁴ are —C(H); and the remaining variables are as set forth in the first aspect.

In a nineteenth aspect, disclosed herein is a compound represented by Formula (A-I) or a salt thereof, wherein M², R⁷, R⁸, m, R³, Z¹, Z², Z³, and Z⁴ are as set forth in any one of aspects 1-18 wherein M¹ is

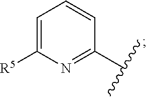

and the remaining variables are as set forth in the first aspect.

In a twentieth aspect, disclosed herein is a compound represented by Formula (A-I) or a salt thereof, wherein M¹, R⁷, R⁸, m, R³, Z¹, Z², Z³, and Z⁴ are as set forth in any one of aspects 1-18 wherein M² is

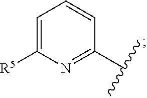

and the remaining variables are as set forth in the first aspect.

In a twenty-first aspect, disclosed herein is a compound represented by Formula (A-I) or a salt thereof, wherein M¹, M², R⁷, R⁸, m, R³, Z¹, Z², Z³, and Z⁴ are as set forth in any one of aspects 1-20 wherein R⁵ is hydrogen, halogen, or C₁-C₃ alkyl optionally substituted with halogen; and the remaining variables are as set forth in the first aspect.

In a twenty-second aspect, disclosed herein is a compound represented by Formula (A-I) or a salt thereof, wherein M¹, M², R⁷, R⁸, m, R³, Z¹, Z², Z³, and Z⁴ are as set forth in any one of aspects 1-20 wherein R⁵ is hydrogen or C₁-C₃ alkyl; and the remaining variables are as set forth in the first aspect.

In a twenty-third aspect, disclosed herein is a compound represented by Formula (A-I) or a salt thereof, wherein M¹, M², R⁷, R⁸, m, R³, Z¹, Z², Z³, and Z⁴ are as set forth in any one of aspects 1-20 and wherein R⁵ is methyl; and the remaining variables are as set forth in the first aspect.

In a twenty-fourth aspect, disclosed herein is a compound represented by Formula (A-I) or a salt thereof, wherein one of M¹ and M² is

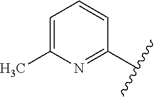 [SMILES: *c1cccc(C)n1]

and the other of M¹ and M² is selected from:

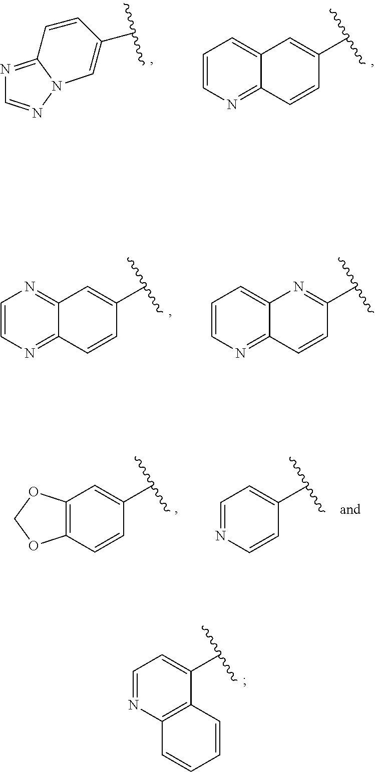

and the remaining variables are as set forth in the first aspect.

In a twenty-fifth aspect, disclosed herein is a compound represented by Formula (A-I) or a salt thereof, wherein M¹ is as set forth in aspect 24 and wherein M² is

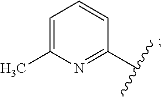 [SMILES: *c1cccc(C)n1]

and the remaining variables are as set forth in the first aspect.

In a twenty-sixth aspect, disclosed herein is a compound represented by Formula (A-I) or a salt thereof, wherein M¹, M², R⁷, R⁸, m, R³, R⁵, Z¹, Z², Z³, and Z⁴ are as set forth in any one of aspects 1-25 and wherein R¹ and R² are, at each occurrence, independently selected from hydrogen, halogen, —OR¹¹, —SR¹¹, —N(R¹¹)₂, phenyl, and —C₁-C₆ alkyl wherein said C₁-C₆ alkyl is optionally substituted with one or more substituents independently selected from halogen, —OR¹¹, —SR¹¹, —S(O)R¹⁰, —S(O)₂R¹¹, —S(O)₂N(R¹¹)₂,

—N(R¹¹)₂, —C(O)R¹⁰, —C(O)N(R¹¹)₂, —N(R¹¹)C(O)R¹⁰, —C(O)OR¹¹, and —OC(O)R¹⁰; and the remaining variables are as set forth in the first aspect.

In a twenty-seventh aspect, disclosed herein is a compound represented by Formula (A-I) or a salt thereof, wherein M¹, M², R⁷, R⁸, m, R³, R⁵, Z¹, Z², Z³, and Z⁴ are as set forth in any one of aspects 1-25 and wherein R¹ and R² are independently selected at each occurrence from hydrogen, phenyl, and —C₁-C₃ alkyl wherein said —C₁-C₃ alkyl is optionally substituted with one or more substituents independently selected from halogen,

—OR¹¹, and —C(O)OR¹¹; and the remaining variables are as set forth in the first aspect.

In a twenty-eighth aspect, disclosed herein is a compound represented by Formula (A-I) or a salt thereof, wherein M¹, M², R⁷, R⁸, m, R³, R⁵, Z¹, Z², Z³, and Z⁴ are as set forth in any one of aspects 1-25 and wherein R¹ and R² are independently selected at each occurrence from hydrogen, —CH₃, —CH₂OH, CH₂CO₂CH₃, and phenyl; and the remaining variables are as set forth in the first aspect.

In a twenty-ninth aspect, disclosed herein is a compound represented by Formula (A-I) or a salt thereof, wherein M¹, M², R⁷, R⁸, m, R³, R⁵, Z¹, Z², Z³, and Z⁴ are as set forth in any one of aspects 1-25 and wherein R and R² are each hydrogen; and the remaining variables are as set forth in the first aspect.

In a thirtieth aspect, disclosed herein is a compound represented by Formula (A-I) or a salt thereof, wherein M¹, M², R⁷, R⁸, m, R¹, R², R³, R⁵, Z¹, Z², Z³, and Z⁴ are as set forth in any one of aspects 1-29 and wherein s is zero; and the remaining variables are as set forth in the first aspect.

In a thirty-first aspect, disclosed herein is a compound represented by Formula (A-I) or a salt thereof, wherein M¹, M², R⁷, R⁸, m, R¹, R², R³, R⁵, Z¹, Z², Z³, and Z⁴ are as set forth in any one of aspects 1-29 and wherein s is one; and the remaining variables are as set forth in the first aspect.

In a thirty-second aspect, disclosed herein is a compound represented by Formula (A-I) or a salt thereof, wherein M¹, M², R⁷, R⁸, m, R¹, R², R³, R⁵, Z¹, Z², Z³, and Z⁴ are as set forth in any one of aspects 1-29 and wherein s is one and n is two or three; and the remaining variables are as set forth in the first aspect.

In a thirty-third aspect, disclosed herein is a compound represented by Formula (A-I) or a salt thereof, wherein M¹, M², R⁷, R⁸, m, R¹, R², R³, R⁵, Z¹, Z², Z³, and Z⁴ are as set forth in any one of aspects 1-29 and wherein s is zero or one and n is one; and the remaining variables are as set forth in the first aspect.

In a thirty-fourth aspect, disclosed herein is a compound represented by Formula (A-I) or a salt thereof, wherein M¹, M², R⁷, R⁸, m, R¹, R², R³, R⁵, Z¹, Z², Z³, and Z⁴ are as set forth in any one of aspects 1-29 and wherein s is zero or one and n is two; and the remaining variables are as set forth in the first aspect.

In a thirty-fifth aspect, disclosed herein is a compound represented by Formula (A-I) or a salt thereof, wherein M¹, M², R⁷, R⁸, m, R¹, R², R³, R⁵, Z¹, Z², Z³, and Z⁴ are as set forth in any one of aspects 1-29 and wherein s is zero or one and n is three; and the remaining variables are as set forth in the first aspect.

In a thirty-sixth aspect, disclosed herein is a compound represented by Formula (A-I) or a salt thereof, wherein M¹, M², R⁷, R⁸, m, s, n R¹, R², R³, R⁵, Z¹, Z², Z³, and Z⁴ are as set forth in any one of aspects 1-35 and wherein Y is selected from —O— and —N(R⁹)— and R⁹ is, at each occurrence, independently selected from: hydrogen; and —C₁-C₆ alkyl optionally substituted with one or more substituents independently selected from halogen, —OR⁴¹, —SR⁴¹, —S(O)R⁴⁰, —S(O)₂R⁴¹, —S(O)₂N(R⁴¹)₂, —N(R⁴¹)₂, —C(O)R⁴⁰, —C(O)N(R⁴¹)₂, —N(R⁴¹)C(O)R⁴⁰, —C(O)OR⁴¹, and —OC(O)R⁴⁰; and the remaining variables are as set forth in the first aspect.

In a thirty-seventh aspect, disclosed herein is a compound represented by Formula (A-I) or a salt thereof, wherein M¹, M², R⁷, R⁸, m, s, n R¹, R², R³, R⁵, Z¹, Z², Z³, and Z⁴ are as set forth in any one of aspects 1-35 and wherein Y is selected from —O— and

—N(R⁹)— and R⁹ is, at each occurrence, independently selected from: hydrogen; and unsubstituted-C₁-C₆ alkyl; and the remaining variables are as set forth in the first aspect.

In a thirty-eight aspect, disclosed herein is a compound represented by Formula (A-I) or a salt thereof, wherein M¹, M², R⁷, R⁸, m, s, n R¹, R², R³, R⁵, Z¹, Z², Z³, and Z⁴ are as set forth in any one of aspects 1-35 and wherein Y is selected from O, N(H), and N(Me); and unsubstituted —C₁-C₆ alkyl; and the remaining variables are as set forth in the first aspect.

In a thirty-ninth aspect, disclosed herein is a compound represented by Formula (A-I) or a salt thereof, wherein M¹, M², R⁷, R⁸, m, s, n R¹, R², R³, R⁵, R⁹, Y, Z¹, Z², Z³, and Z⁴ are as set forth in any one of aspects 1-38 and wherein w is zero; and the remaining variables are as set forth in the first aspect.

In a fortieth aspect, disclosed herein is a compound represented by Formula (A-I) or a salt thereof, wherein M¹, M², R⁷, R⁸, m, s, n R¹, R², R³, R⁵, R⁹, Y, Z¹, Z², Z³, and Z⁴ are as set forth in any one of aspects 1-38 and wherein w is 1, 2, 3, 4, or 5; and the remaining variables are as set forth in the first aspect.

In a forty-first aspect, disclosed herein is a compound represented by Formula (A-I) or a salt thereof, wherein M¹, M², R⁷, R⁸, m, s, n R¹, R², R³, R⁵, R⁹, Y, Z¹, Z², Z³, and Z⁴ are as set forth in any one of aspects 1-38 and wherein w is 1, 2, or 3; and the remaining variables are as set forth in the first aspect.

In a forty-second aspect, disclosed herein is a compound represented by Formula (A-I) or a salt thereof, wherein M¹, M², R⁷, R⁸, m, s, n R¹, R², R³, R⁵, R⁹, Y, Z¹, Z², Z³, and Z⁴ are as set forth in any one of aspects 1-38 and wherein w is 1 or 2; and the remaining variables are as set forth in the first aspect.

In a forty-third aspect, disclosed herein is a compound represented by Formula (A-I) or a salt thereof, wherein M¹, M², R⁷, R⁸, m, s, n R¹, R², R³, R⁵, R⁹, Y, Z¹, Z², Z³, and Z⁴ are as set forth in any one of aspects 1-38 having formula (A-IA), (A-IB), (A-IC), (A-ID) or (A-IE):

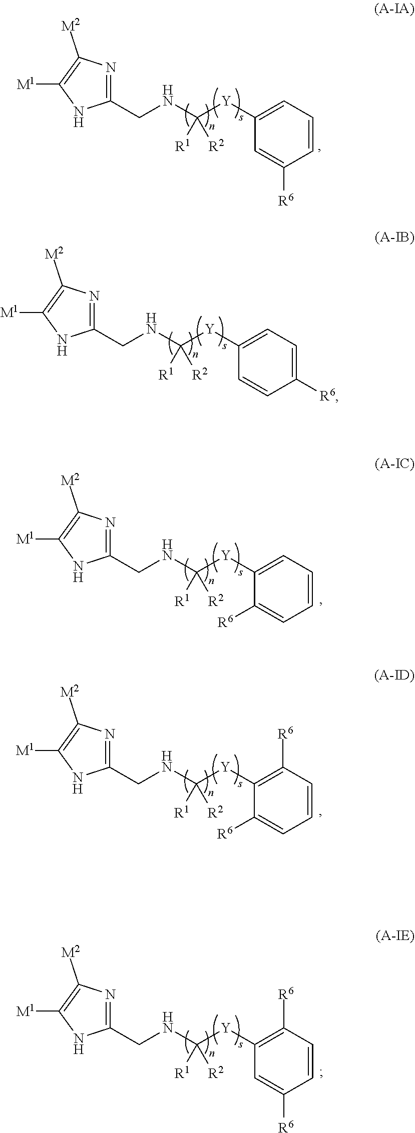

or a salt of any one of formula (A-IA), (A-IB), (A-IC), (A-ID) or (A-IE); and the remaining variables are as set forth in the first aspect.

In a forty-fourth aspect, disclosed herein is a compound represented by Formula (A-I) wherein M¹, M², R⁷, R⁸, m, s, n R¹, R², R³, R⁵, R⁹, Y, Z¹, Z², Z³, and Z⁴ are as set forth in any one of aspects 1-38 having formula (A-IC) or (A-ID)

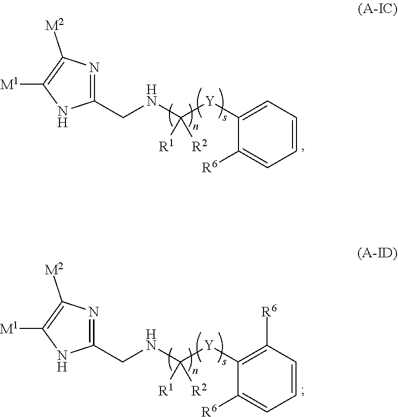

or a salt of any one of formula (A-IC), or (A-ID); and the remaining variables are as set forth in the first aspect.

In a forty-fifth aspect, disclosed herein is a compound represented by Formula (A-I) wherein M¹, M², R⁷, R⁸, m, s, n R¹, R², R³, R⁵, R⁹, Y, Z¹, Z², Z³, and Z⁴ are as set forth in any one of aspects 1-38 having formula (A-IF):

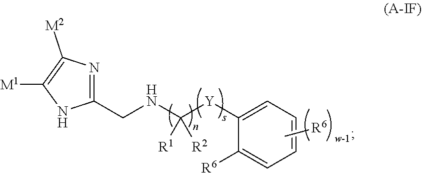

wherein w is 1, 2, 3, or 4; or a salt thereof); and the remaining variables are as set forth in the first aspect.

In a forty-sixth aspect, disclosed herein is a compound represented by Formula (A-IF) or a salt thereof, wherein M¹, M², R⁷, R⁸, m, s, n, R¹, R², R³, R⁵, R⁹, Y, Z¹, Z², Z³, and Z⁴ are as set forth in any one of aspects 1-38 and w is 2 or 3; and the remaining variables are as set forth in the first aspect.

In a forty-seventh aspect, disclosed herein is a compound represented by Formula (A-IF) or a salt thereof, wherein M¹, M², R⁷, R⁸, m, s, n, R¹, R², R³, R⁵, R⁹, Y, Z¹, Z², Z³, and Z⁴ are as set forth in any one of aspects 1-38 and w is 2; and the remaining variables are as set forth in the first aspect.

In a forty-eighth aspect, disclosed herein is a compound represented by Formula (A-I), (A-IA), (A-IB), (A-IC), (A-ID), (A-IE), or (A-IF) or a salt thereof, wherein M¹, M², R⁷, R⁸, m, s, n, w, R¹, R², R³, R⁵, R⁹, Y, Z¹, Z², Z³, and Z⁴ are as set forth in any one of aspects 1-47 and R⁶ is independently selected at each occurrence from:

halogen, —OR²¹, —N(R²¹)₂ and —CN; and

C₁-C₆ alkyl optionally substituted with one or more substituents independently selected from halogen, —OR²¹, —SR²¹, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)₂, —NO₂, ═O, ═S, ═N(R²¹), —CN, a C₃-C₁₀ carbocycle, and a 3- to 10-membered heterocycle wherein said C₃-C₁₀ carbocycle and said 3- to 10-membered heterocycle are optionally substituted with one or more substituents independently selected from R^{X}; and

a C₃-C₁₀ carbocycle and a 3- to 10-membered heterocycle, each of which is optionally substituted with one or more substituents independently selected from halogen,

—OR²⁰, —OH, —SR²⁰, —SH, —N(R²¹)₂, —C(O)R²⁰, —C(O)N(R²¹)₂, —N(R²¹)C(O)R²⁰, —C(O)OR²¹,
—OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)₂, —NO₂, ═O, ═S, ═N(R²¹), —CN, —C₂-C₆ alkenyl, —C₂-C₆ alkynyl and C₁-C₆ alkyl wherein said C₁-C₆ alkyl is optionally substituted with one or more substituents independently selected from R^{Y};

and the remaining variables are as set forth in the first aspect.

In a forty-ninth aspect, disclosed herein is a compound represented by Formula (A-I), (A-IA), (A-IB), (A-IC), (A-ID), (A-IE) or (A-IF) or a salt thereof, wherein M¹, M², R⁷, R⁸, m, s, n, w, R¹, R², R³, R⁵, R⁹, Y, Z¹, Z², Z³, and Z⁴ are as set forth in any one of aspects 1-47 and R⁶ is independently selected at each occurrence from:

halogen, —OR²¹, and —N(R²¹)₂;

C₁-C₆ alkyl optionally substituted with one or more substituents independently selected from halogen, —OR²¹, —SR²¹, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)₂, —NO₂, ═O, ═S, ═N(R²¹), —CN, a C₃-C₁₀ carbocycle, and a 3- to 10-membered heterocycle, wherein said C₃-C₁₀ carbocycle and said 3- to 10-membered heterocycle are optionally substituted with one or more substituents independently selected from R^{X}; and

phenyl and a 6-membered heterocycle comprising 1, 2, or 3 ring heteroatoms selected from nitrogen, each of which is optionally substituted with one or more substituents independently selected from halogen, —OR²⁰, —OH, —SR²⁰, —SH, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹,

—S(O)₂N(R²¹)₂, —OC(O)OR²¹, —OC(O)N(R²¹)₂, —NR²¹C(═O)OR²¹, —N(R²¹)C(O)N(R²¹)₂, —NO₂, ═O, ═S, ═N(R²¹), —CN, —C₂-C₆ alkenyl, —C₂-C₆ alkynyl and C₁-C₆ alkyl wherein said C₁-C₆ alkyl is optionally substituted with one or more substituents independently selected from R^{Y};

and the remaining variables are as set forth in the first aspect.

In some such aspects, at least one R⁶ is phenyl optionally substituted with one or more substituents independently selected from halogen, —OR²⁰, —OH, —SR²⁰, —SH, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²)C(O)N(R²¹)₂, —NO₂, ═O, ═S, ═N(R²¹), —CN, —C₂-C₆ alkenyl, —C₂-C₆ alkynyl and C₁-C₆ alkyl, wherein said C₁-C₆ alkyl is optionally substituted with one or more substituents independently selected from R^{Y}.

In a fiftieth aspect, disclosed herein is a compound represented by Formula (A-I), (A-IA), (A-IB), (A-IC), (A-ID), (A-IE) or (A-IF) or a salt thereof, wherein M¹, M², R⁷, R⁸, m, s, n, w, R¹, R², R³, R⁵, R⁹, Y, Z¹, Z², Z³, and Z⁴ are as set forth in any one of aspects 1-47 and R⁶ is independently selected at each occurrence from:
halogen, —OR²¹, and —N(R²¹)₂;
C₁-C₆ alkyl optionally substituted with halogen; and
a C₃-C₁₀ carbocycle and a 3- to 10-membered heterocycle, each of which is optionally substituted with C₁-C₆ alkyl, wherein said C₁-C₆ alkyl is optionally substituted with halogen;

and the remaining variables are as set forth in the first aspect.

In a fifty-first aspect, disclosed herein is a compound represented by Formula (A-I), (A-IA), (A-IB), (A-IC), (A-ID), (A-IE) or (A-IF) or a salt thereof, wherein M¹, M², R⁷, R⁸, m, s, n, w, R¹, R², R³, R⁵, R⁹, Y, Z¹, Z², Z³, and Z⁴ are as set forth in any one of aspects 1-47 and R⁶ is independently selected at each occurrence from:
halogen, and —OR²¹;
C₁-C₆ alkyl optionally substituted with halogen; and
phenyl and a 6-membered heterocycle comprising 1, 2, or 3 ring heteroatoms selected from nitrogen, each of which is optionally substituted with C₁-C₆ alkyl wherein said C₁-C₆ alkyl is optionally substituted with one or more substituents independently selected from R^{Y};

and the remaining variables are as set forth in the first aspect.

In a fifty-second aspect, disclosed herein is a compound represented by Formula (A-I), (A-IA), (A-IB), (A-IC), (A-ID), (A-IE) or (A-IF) or a salt thereof, wherein M¹, M², R⁶, R⁷, R⁸, m, s, n, w, R¹, R², R³, R⁵, R⁹, Y, Z¹, Z², Z³, and Z⁴ are as set forth in any one of aspects 1-51 and R²¹ is C₁-C₃alkyl, phenyl, or a 6-membered heterocycle comprising 1, 2, or 3 ring heteroatoms selected from nitrogen; and the remaining variables are as set forth in the first aspect.

In a fifty-third aspect, disclosed herein is a compound represented by Formula (A-I), (A-IA), (A-IB), (A-IC), (A-ID), (A-IE) or (A-IF) or a salt thereof, wherein M¹, M², R⁶, R⁷, R⁸, m, s, n, w, R¹, R², R³, R⁵, R⁹, R²¹, Y, Z¹, Z², Z³, and Z⁴ are as set forth in any one of aspects 1-52 and R on the phenyl or heterocycle of R⁶ is selected from halogen; and the remaining variables are as set forth in the first aspect.

In a fifty-fourth aspect, disclosed herein is a compound represented by Formula (A-I), (A-IA), (A-IB), (A-IC), (A-ID), (A-IE) or (A-IF) or a salt thereof, wherein M¹, M², R⁶, R⁷, R⁸, m, s, n, w, R¹, R², R³, R⁵, R⁹, R²¹, Y, Z¹, Z², Z³, and Z⁴ are as set forth in any one of aspects 1-52 and R on the phenyl or heterocycle of R⁶ is selected from fluorine or chlorine; and the remaining variables are as set forth in the first aspect.

In a fifty-fifth aspect, disclosed herein is a compound represented by Formula (A-I), (A-IA), (A-IB), (A-IC), (A-ID), (A-IE) or (A-IF) or a salt thereof, wherein M¹, M², R⁷, R⁸, m, s, n, w, R¹, R², R³, R⁵, R⁹, R²¹, Y, Z¹, Z², Z³, and Z⁴ are as set forth in any one of aspects 1-54 and R⁶ is independently selected at each occurrence from: F, Cl,

—OCH₃, —CF₃, —CN, —CH₃, —CH₂CH₃, —CH(CH₃)₂, —OCF₃, —CH₂CF₃, —CH(OH)(CF₃), N(CH₃)₂, pyridyl, cyclohexyl, cyclopentyl, —O-phenyl, —O-pyridyl, and phenyl optionally substituted with one or more substituents independently selected from F, and —CH₂NH₂; and the remaining variables are as set forth in the first aspect. In some such aspects, R⁶ does not comprise cyano.

In a fifty-sixth aspect, disclosed herein is a compound represented by Formula (A-I) or a salt thereof, wherein M¹, M², R⁷, R⁸, m, s, n, R¹, R², R³, R⁵, R⁹, R²¹, Y, Z¹, Z², Z³, and Z⁴ are as set forth in any one of aspects 1-55 and is:

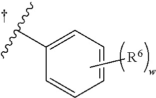

is:

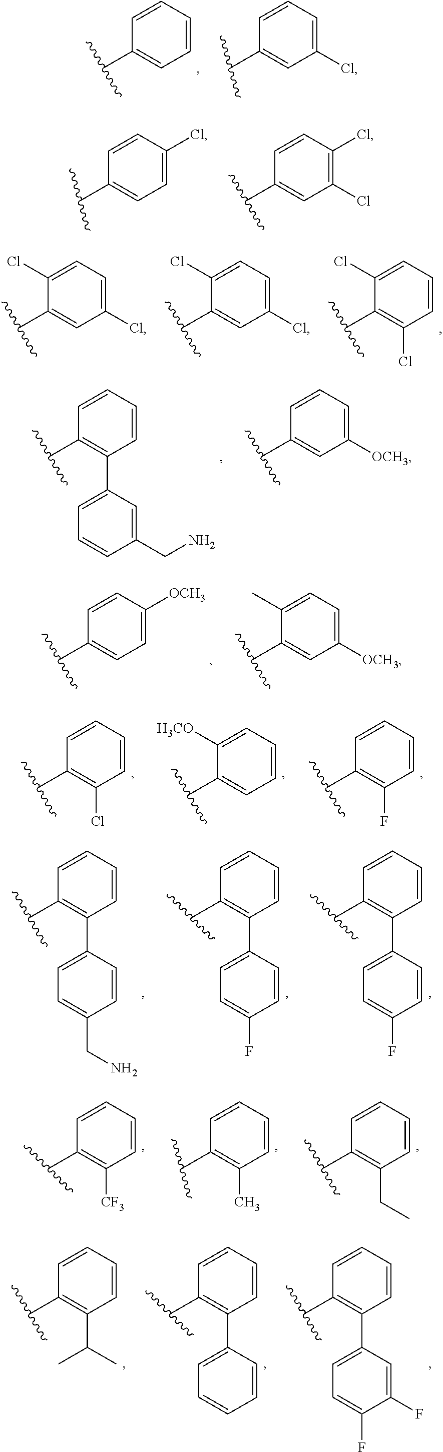

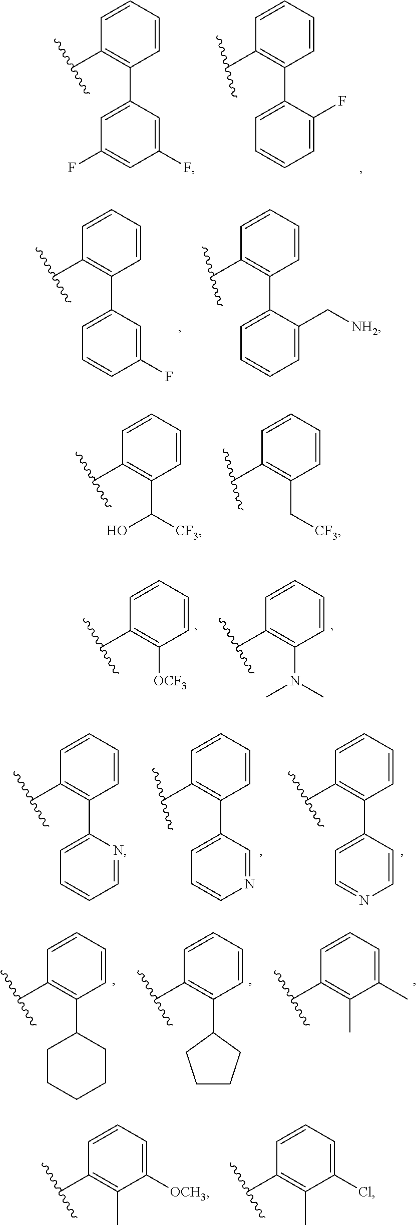

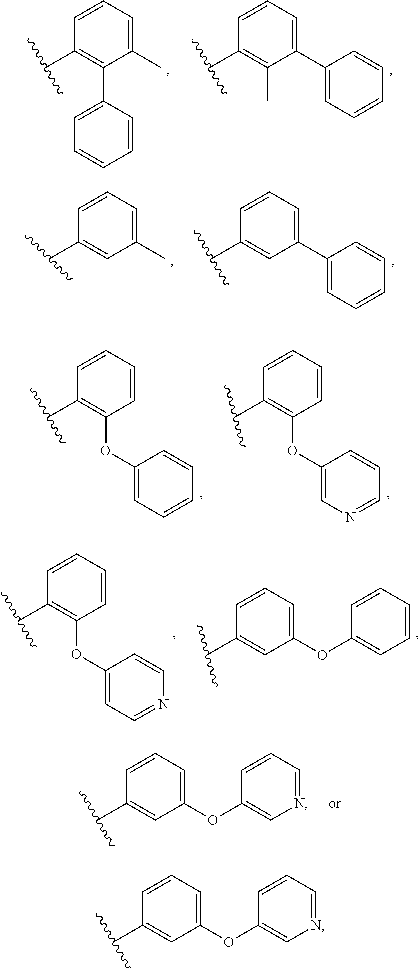

wherein [symbol] represents the point of attachment to

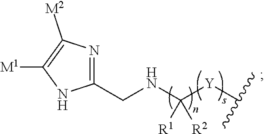

and the remaining variables are as set forth in the first aspect.

In a fifty-seventh aspect, disclosed herein is a compound represented by Formula (A-I) or a salt thereof, wherein M¹, M², R⁷, R⁸, m, R³, R⁵, R⁹, R²¹, Z¹, Z², Z³, and Z⁴ are as set forth in any one of aspects 1-55 and wherein

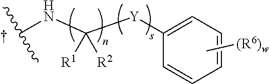

is:

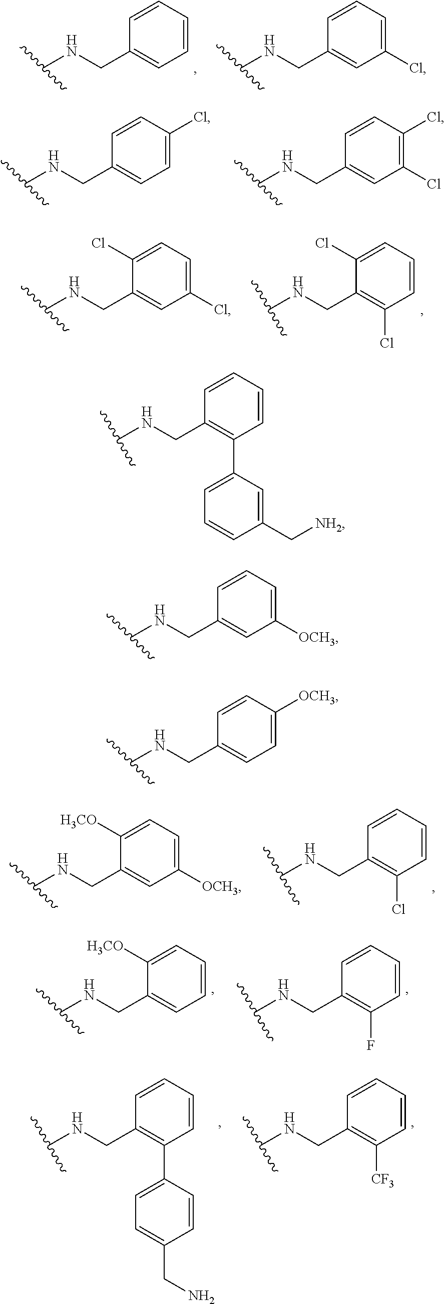

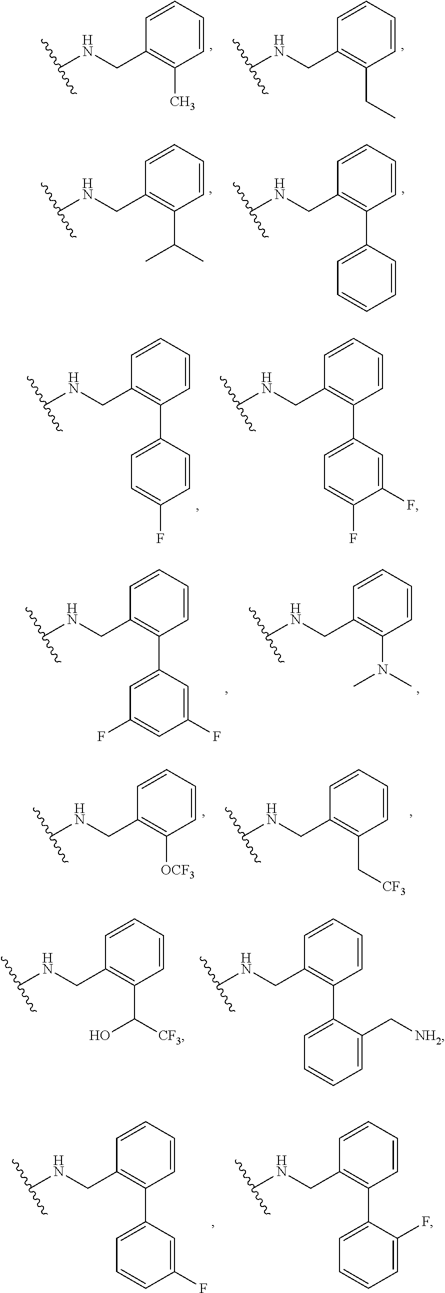

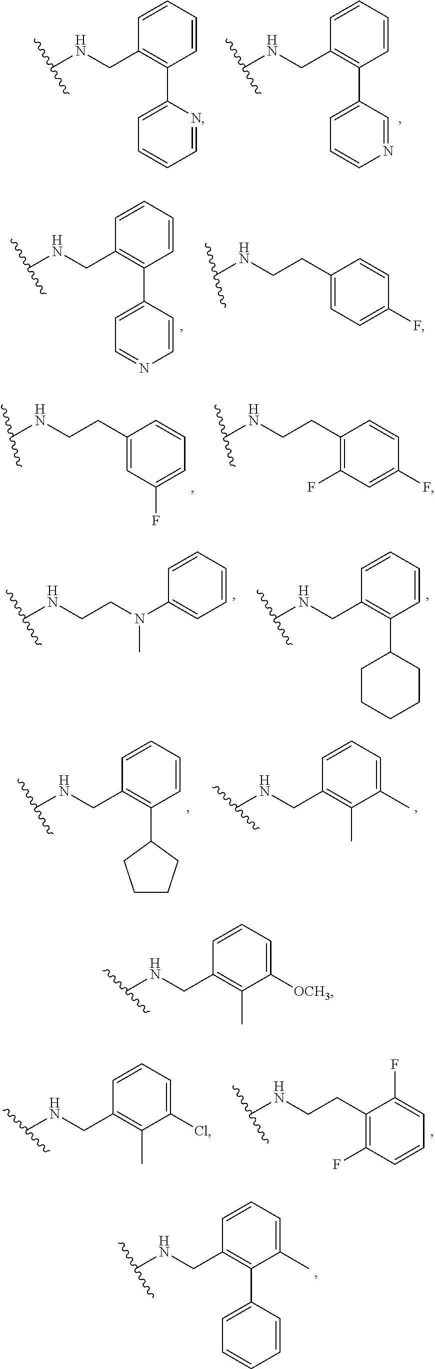

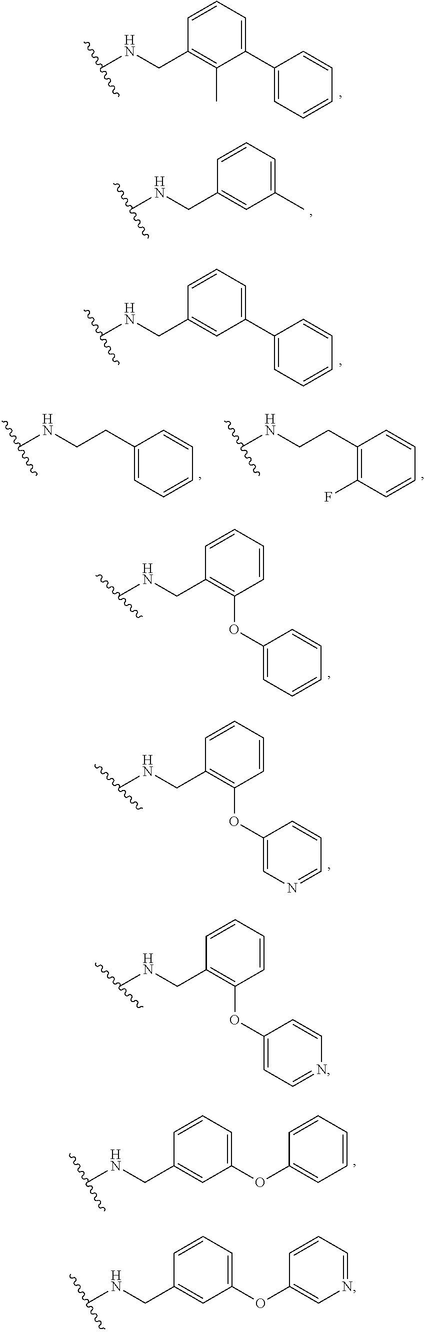

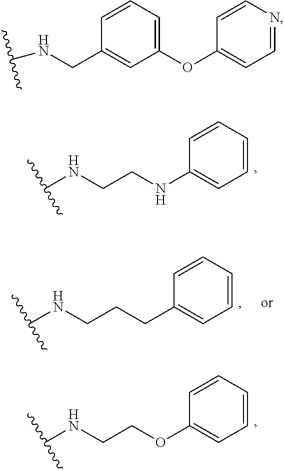

wherein [symbol] represents the point of attachment to

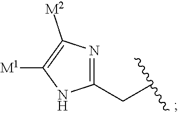

and the remaining variables are as set forth in the first aspect.

In another aspect, the TGFβR1 inhibitor is a compound from Table 3.

TGFβR1 Inhibitors of Formula (B-I)

In some aspects, the TGFβR1 inhibitor is a compound of Formula (B-I):

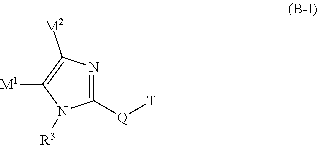

wherein:

M¹ and M² are independently selected from

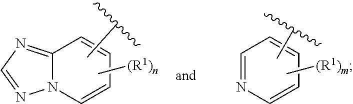

R¹ and R² are independently selected at each occurrence from:
a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, and —CN;
—C₁-C₁₀ alkyl, —C₂-C₁₀ alkenyl, and —C₂-C₁₀ alkynyl, each of which is optionally substituted at each occurrence with one or more substituents independently selected from a halogen, —OR¹⁰, —SR¹⁰, —N(R¹⁰)₂, —C(O)R¹⁰, —C(O)N(R¹⁰)₂—N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, a C₃-C₁₀ carbocycle, and a 3- to 10-membered heterocycle; and
a C₃-C₁₀ carbocycle and a 3- to 10-membered heterocycle, each of which is optionally substituted with one or more substituents independently selected from a halogen, —OR¹⁰, —SR¹⁰, —N(R¹⁰)₂, —C(O)R¹⁰, —C(O)N(R¹⁰)₂, —N(R^{c})C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, —C₁-C₆ alkyl, —C₂-C₆ alkenyl, and —C₂-C₆ alkynyl;

R³ is selected from hydrogen and —C₁-C₁₀ alkyl optionally substituted with one or more substituents independently selected from a halogen, —NO₂, ═O, ═S, ═N(R¹⁰), —CN, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, and —OC(O)R¹⁰;

n and m are independently selected from 0, 1, 2, 3, and 4;

Q is selected from a bond, —(CR¹⁰₂)ₚ—, —(CR¹⁰₂)_{q}C(═O)(CR¹⁰₂)_{q}—, —(CR¹⁰₂)_{q}C(═S)(CR¹⁰₂)_{q}—, —(CR¹⁰₂)_{q}C(═NR¹⁰)(CR¹⁰₂)_{q}—, —(CR¹⁰₂)_{q}O(CR¹⁰₂)_{q}—, —(CR¹⁰₂)_{q}S(CR¹⁰₂)_{q}—, —(CR¹⁰₂)_{q}N(R¹⁰)(CR¹⁰₂)_{q}—, —(CR¹⁰₂)_{q}OC(═O)O(CR¹⁰₂)_{q}—, —(CR¹⁰₂)_{q}C(═O)N(R¹⁰)(CR¹⁰₂)_{q}—, —(CR¹⁰₂)_{q}N(R¹⁰)C(═O)(CR¹⁰₂)_{q}—, and —(CR¹⁰₂)_{q}N(R¹⁰)SO₂(CR¹⁰₂)_{q}—;

p is selected from 1, 2, 3, 4, and 5;

q is independently selected at each occurrence from 0, 1, 2, 3, 4, and 5;

T is selected from an optionally substituted saturated C₃-C₇ carbocycle, an optionally substituted C₅₋₁₂ bicyclic carbocycle, and an optionally substituted 4- to 12-membered heterocycle, wherein T is optionally substituted with one or more substituents independently selected at each occurrence from R¹³;

R¹³ is independently selected at each occurrence from:
a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), and —CN;
—C₁-C₁₀ alkyl, —C₂-C₁₀ alkenyl, and —C₂-C₁₀ alkynyl, each of which is optionally substituted at each occurrence with one or more substituents independently selected from a halogen, —OR¹⁰, —SR¹⁰, —N(R¹⁰)₂, —C(O)R¹⁰, —C(O)N(R¹⁰), —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, a C₃-C₁₀ carbocycle, and a 3- to 10-membered heterocycle; and
a C₃-C₁₀ carbocycle and a 3- to 10-membered heterocycle, each of which is optionally substituted with one or more substituents independently selected from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, —C₁-C₆ alkyl, —C₂-C₆ alkenyl, and —C₂-C₆ alkynyl; and

R¹⁰ is independently selected at each occurrence from:
hydrogen;
—C₁-C₁₀ alkyl, —C₂-C₁₀ alkenyl, and —C₂-C₁₀ alkynyl, each of which is optionally substituted at each occurrence with one or more substituents independently selected from a halogen, —OH, —CN, —NO₂, —NH₂, ═O, ═S, —O—C₁-C₁₀ alkyl, C₃-C₁₂ carbocycle, and a 3- to 12-membered heterocycle; and
a C₃-C₁₂ carbocycle and a 3- to 12-membered heterocycle, each of which is optionally substituted at each occurrence with one or more substituents independently selected from a halogen, —OH, —CN, —NO₂, —NH₂, ═O, ═S, —C₁-C₁₀ alkyl, —O—C₁-C₁₀ alkyl, and —C₁-C₁₀ haloalkyl;

or a salt thereof.

In some embodiments one of M¹ and M² is

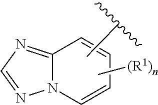

and the other of M¹ and M² is

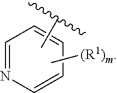

For example, M¹ may be

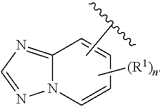

In some embodiments, the compound or salt is represented by Formula (B-Ia):

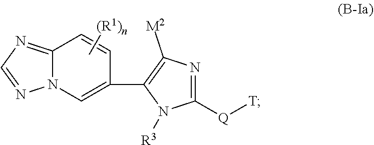

or a salt thereof.

In some embodiments, M² is

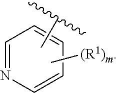

In some embodiments, the compound or salt is represented by Formula (B-Ib):

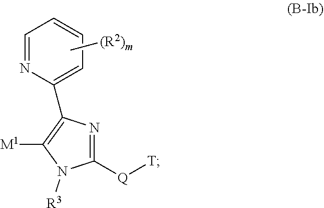

or a salt thereof.

In some embodiments, M¹ is

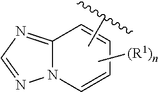

### and M² is

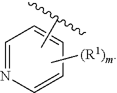

In other embodiments, M¹ is

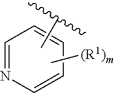

### and M² is

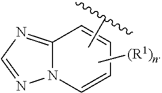

In some embodiments, the compound or salt is represented by Formula (B-Ic) or Formula (B-Id):

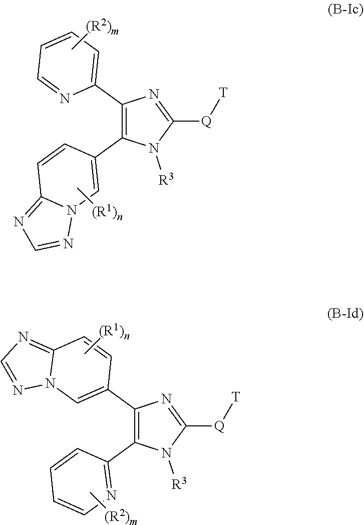

or a salt thereof.

In some embodiments, R³ for a compound or salt of any of Formulas (B-I), (B-Ia), (B-Ib), (B-Ic), and (B-Id) is selected from hydrogen and —C₁-C₁₀ alkyl optionally substituted with one or more substituents independently selected from a halogen, —NO₂, —CN, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, and —OC(O)R¹⁰. For example, for a compound or salt of any of Formulas (B-I), (B-Ia), (B-Ib), (B-Ic), and (B-Id), R³ may be selected from hydrogen and —C₁-C₁₀ alkyl optionally substituted with one or more substituents independently selected from a halogen, —NO₂, —CN, —OR¹⁰, —SR¹⁰, and —N(R¹⁰)₂. In some embodiments, for a compound or salt of any of Formulas (B-I), (B-Ia), (B-Ib), (B-Ic), and (B-Id), R³ is hydrogen.

In some embodiments, for a compound or salt of any of Formulas (B-I), (B-Ia), (B-Ib), (B-Ic), and (B-Id), n is 0. In other embodiments, for a compound or salt of any of Formulas (B-I), (B-Ia), (B-Ib), (B-Ic), and (B-Id), n is selected from 1, 2, 3, and 4. In some embodiments, for a compound or salt of any of Formulas (B-I), (B-Ia), (B-Ib), (B-Ic), and (B-Id), each R¹ is independently selected from a halogen, —OR¹⁰, —SR¹⁰, —N(R¹⁰)₂,

—C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, and —CN; and —C₁-C₁₀ alkyl, —C₂-C₁₀ alkenyl, and —C₂-C₁₀ alkynyl, each of which is optionally substituted at each occurrence with one or more substituents independently selected from a halogen, —OR¹⁰, —SR¹⁰,
—N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, a C₃-C₁₀ carbocycle, and a 3- to 10-membered heterocycle. In certain embodiments, for a compound or salt of any of Formulas (B-I), (B-Ia), (B-Ib), (B-Ic), and (B-Id), each R¹ is selected from a halogen, —OR¹⁰, —SR¹⁰, —N(R¹⁰)₂, —NO₂, and —CN; and —C₁-C₁₀ alkyl optionally substituted at each occurrence with one or more substituents independently selected from a halogen, —OR¹⁰, —SR¹⁰, —N(R¹⁰)₂, —C(O)R¹⁰, —NO₂, and —CN. In some embodiments, for a compound or salt of any of Formulas (B-I), (B-Ia), (B-Ib), (B-Ic), and (B-Id), each R¹ is independently selected from a —C₁-C₁₀ alkyl optionally substituted at each occurrence with one or more substituents independently selected from a halogen, —OR¹⁰,
—SR¹⁰, —N(R¹⁰)₂, —C(O)R¹⁰, —NO₂, and —CN.

In some embodiments, for a compound or salt of any of Formulas (B-I), (B-Ia), (B-Ib), (B-Ic), and (B-Id), m is 0. In other embodiments, for a compound or salt of any of Formulas (B-I), (B-Ia), (B-Ib), (B-Ic), and (B-Id), m is selected from 1, 2, 3, and 4. In some embodiments, for a compound or salt of any of Formulas (B-I), (B-Ia), (B-Ib), (B-Ic), and (B-Id), each R² is independently selected from a halogen, —OR¹⁰, —SR¹⁰, —N(R¹⁰)₂,

—C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, and —CN; and —C₁-C₁₀ alkyl, —C₂-C₁₀ alkenyl, and —C₂-C₁₀ alkynyl, each of which is optionally substituted at each occurrence with one or more substituents independently selected from a halogen, —OR¹⁰, —SR¹⁰,
—N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, a C₃-C₁₀ carbocycle, and a 3- to 10-membered heterocycle. In certain embodiments, for a compound or salt of any of Formulas (B-I), (B-Ia), (B-Ib), (B-Ic), and (B-Id), each R² is independently selected from a halogen, —OR¹⁰, —SR¹⁰, —N(R¹⁰)₂, —NO₂, and —CN; and —C₁-C₁₀ alkyl optionally substituted at each occurrence with one or more substituents independently selected from a halogen, —OR¹⁰, —SR¹⁰, —N(R¹⁰)₂, —C(O)R¹⁰, —NO₂, and —CN. In some embodiments, for a compound or salt of any of Formulas (B-I), (B-Ia), (B-Ib), (B-Ic), and (B-Id), each R² is independently selected from —OR¹⁰ and —C₁-C₁₀ alkyl optionally substituted with one or more substituents independently selected from a halogen, —OR¹⁰,
—SR¹⁰, —N(R¹⁰)₂, —C(O)R¹⁰, —NO₂, and —CN. In some embodiments, for a compound or salt of any of Formulas (B-I), (B-Ia), (B-Ib), (B-Ic), and (B-Id), m is 1 and R² is —CH₃. For example, for a compound or salt of any of Formulas (B-I), (B-Ia), (B-Ib), (B-Ic), and (B-Id), M² may be

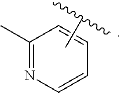

In a particular example, for a compound or salt of any of Formulas (B-I), (B-Ia), (B-Ib), (B-Ic), and (B-Id), M² is

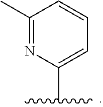 [SMILES: *c1cccc(C)n1]

In some embodiments, the compound of Formula (B-I) is represented by Formula (B-Ie).

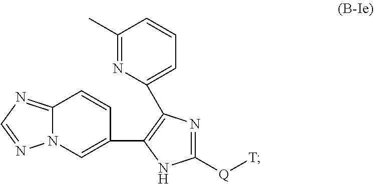

or a salt thereof.

In some embodiments, for a compound or salt of any of Formulas (B-I), (B-Ia), (B-Ib), (B-Ic), (B-Id), and (B-Ie), Q is selected from —(CR¹⁰₂)ₚ—, —(CR¹⁰₂)_{q}O(CR¹⁰₂)_{q}—, —(CR¹⁰₂)_{q}S(CR¹⁰₂)_{q}—, and —(CR¹⁰₂)_{q}NR¹⁰(CR¹⁰₂)_{q}—, where p is selected from 1, 2, 3, 4, and 5 and q is independently selected at each occurrence from 0, 1, 2, 3, 4, and 5. In some embodiments, for a compound or salt of any of Formulas (B-I), (B-Ia), (B-Ib), (B-Ic),

(B-Id), and (B-Ie), Q is selected from —(CR¹⁰₂)ₚ—. For example, for a compound or salt of any of Formulas (B-I), (B-Ia), (B-Ib), (B-Ic), (B-Id), and (B-Ie), p may be 1 such that Q is
—C(R¹⁰)₂—. Alternatively, for a compound or salt of any of Formulas (B-I), (B-Ia), (B-Ib), (B-Ic), (B-Id), and (B-Ie), Q may be selected from —(CR¹⁰₂)_{q}O(CR¹⁰₂)_{q}—, —(CR¹⁰₂)_{q}S(CR¹⁰₂)_{q}—, and —(CR¹⁰₂)_{q}NR¹⁰(CR¹⁰₂)_{q}—. In some embodiments, for a compound or salt of any of Formulas (B-I), (B-Ia), (B-Ib), (B-Ic), (B-Id), and (B-Ie), Q is —CH₂NH—. In some embodiments, for a compound or salt of any of Formulas (B-I), (B-Ia), (B-Ib),
(B-Ic), (B-Id), and (B-Ie), Q is selected from —(CR¹⁰₂)_{q}NR¹⁰(CR¹⁰₂)_{q}—. In some embodiments, for a compound or salt of any of Formulas (B-I), (B-Ia), (B-Ib), (B-Ic), (B-Id), and (B-Ie), Q is selected from —(CR¹⁰₂)_{q}NR¹⁰—. In some embodiments, for a compound or salt of any of Formulas (B-I), (B-Ia), (B-Ib), (B-Ic), (B-Id), and (B-Ie), Q is selected from —(CR¹⁰₂)_{q}NR¹⁰—. In some embodiments, for a compound or salt of any of Formulas (B-I), (B-Ia), (B-Ib), (B-Ic), (B-Id), and (B-Ie), Q is —CR¹⁰₂NR¹⁰(CR¹⁰₂)₁₋₂—. In some embodiments, for a compound or salt of any of Formulas (B-I), (B-Ia), (B-Ib), (B-Ic), (B-Id), and (B-Ie), Q is —CH₂NHCH₂— or —CH₂NHCH₂CH₂—. In another example, for a compound or salt of any of Formulas (B-I), (B-Ia), (B-Ib), (B-Ic), (B-Id), and (B-Ie), Q is —CH₂NHCH₂—.

In some embodiments, for a compound or salt of any of Formulas (B-I), (B-Ia), (B-Ib), (B-Ic), (B-Id), and (B-Ie), each R¹⁰ is independently selected from hydrogen; and —C₁-C₁₀ alkyl, —C₂-C₁₀ alkenyl, and —C₂-C₁₀ alkynyl, each of which is optionally substituted at each occurrence with one or more substituents independently selected from a halogen, —OH, —CN, —NO₂, and —NH₂. In certain embodiments, for a compound or salt of any of Formulas (B-I), (B-Ia), (B-Ib), (B-Ic), (B-Id), and (B-Ie), R¹⁰ is hydrogen at each occurrence.

In some embodiments, for a compound or salt of any one of Formulas (B-I), (B-Ia), (B-Ib), (B-Ic), (B-Id), and (B-Ie), each R¹⁰ of Q is hydrogen at each occurrence.

In some embodiments, for a compound or salt of any of Formulas (B-I), (B-Ia), (B-Ib), (B-Ic), (B-Id), and (B-Ie), T is selected from a saturated C₃-C₇ carbocycle optionally substituted with one or more substituents independently selected at each occurrence from R¹³. For example, for a compound or salt of any of Formulas (B-I), (B-Ia), (B-Ib), (B-Ic), (B-Id), and (B-Ie), T may be a saturated C₃ carbocycle optionally substituted with one or more substituents independently selected at each occurrence from R¹³. In some embodiments, for a compound or salt of any of Formulas (B-I), (B-Ia), (B-Ib), (B-Ic), (B-Id), and (B-Ie), T may be selected from:

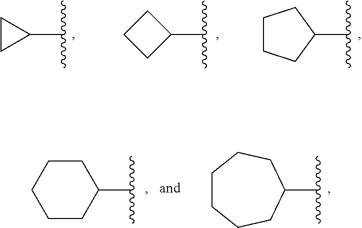

each of which is optionally substituted with one or more substituents independently selected at each occurrence from R¹³. In some embodiments, for a compound or salt of any of Formulas (B-I), (B-Ia), (B-Ib), (B-Ic), (B-Id), and (B-Ie), T is selected from

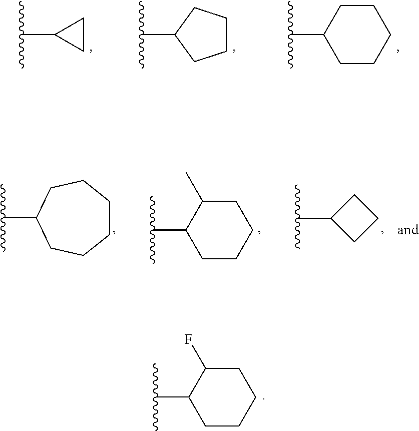

For example, for a compound or salt of any of Formulas (B-I), (B-Ia), (B-Ib), (B-Ic), (B-Id), and (B-Ie), T may be:

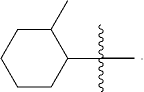 [SMILES: *C1CCCCC1C]

In some embodiments, for a compound or salt of any of Formulas (B-I), (B-Ia), (B-Ib), (B-Ic), (B-Id), and (B-Ie), T is selected from a C₅₋₁₂ bicyclic carbocycle optionally substituted with one or more substituents independently selected at each occurrence from R¹³. In certain embodiments, for a compound or salt of any of Formulas (B-I), (B-Ia), (B-Ib), (B-Ic), (B-Id), and (B-Ie), T is selected from a saturated C₅₋₁₂ bridged carbocycle optionally substituted with one or more substituents independently selected at each occurrence from R¹³, such as an optionally substituted C₅ bridged carbocycle. For example, for a compound or salt of any of Formulas (B-I), (B-Ia), (B-Ib), (B-Ic), (B-Id), and (B-Ie), T may be selected from:

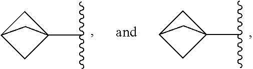 [SMILES: *C12CC(C1)C2 ; *C12CC(C1)C2]

each of which is optionally substituted with one or more substituents independently selected at each occurrence from R¹³.

In other embodiments, for a compound or salt of any of Formulas (B-I), (B-Ia), (B-Ib), (B-Ic), (B-Id), and (B-Ie), T is selected from C₈₋₁₁ bicyclic carbocycle optionally substituted with one or more substituents independently selected at each occurrence from R¹³. In some embodiments, for a compound or salt of any of Formulas (B-I), (B-Ia), (B-Ib), (B-Ic), (B-Id), and (B-Ie), T is a fused bicyclic ring. In certain embodiments, for a compound or salt of any of Formulas (B-I), (B-Ia), (B-Ib), (B-Ic), (B-Id), and (B-Ie), T is selected from a C₈₋₁₁ fused bicyclic carbocycle. In certain embodiments, for a compound or salt of any of Formulas (B-I), (B-Ia), (B-Ib), (B-Ic), (B-Id), and (B-Ie), T is selected from:

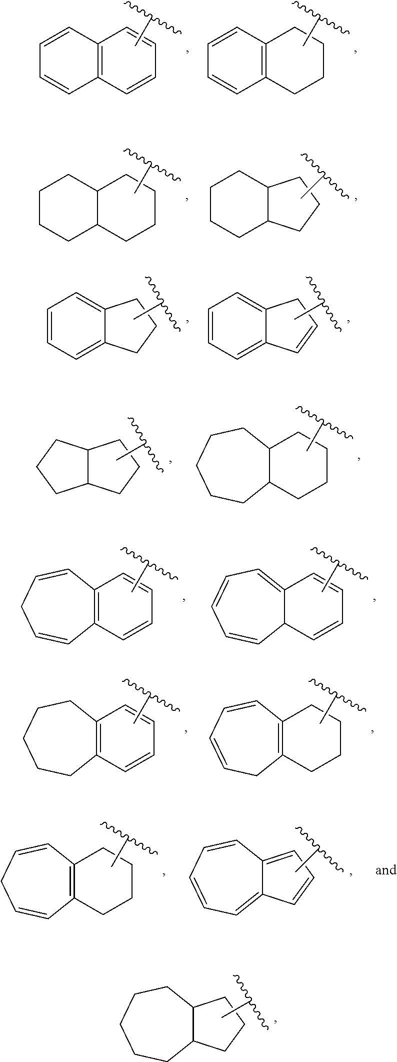

and wherein T is optionally substituted with one or more substituents independently selected at each occurrence from R¹³. For example, for a compound or salt of any of Formulas (B-I), (B-Ia), (B-Ib), (B-Ic), (B-Id), and (B-Ie), T may be selected from:

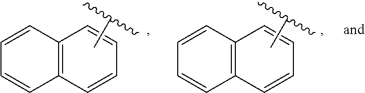

wherein T is optionally substituted with one or more substituents independently selected at each occurrence from R¹³. In certain embodiments, for a compound or salt of any of Formulas (B-I), (B-Ia), (B-Ib), (B-Ic), (B-Id), and (B-Ie), T is

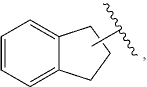

wherein T is optionally substituted with one or more substituents independently selected at each occurrence from R¹³. In some embodiments, for a compound or salt of any of Formulas (B-I), (B-Ia), (B-Ib), (B-Ic), (B-Id), and (B-Ie), T may be selected from:

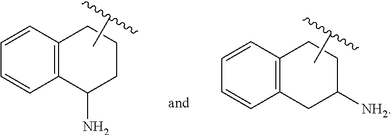

In some embodiments, for a compound or salt of any of Formulas (B-I), (B-Ia), (B-Ib), (B-Ic), (B-Id), and (B-Ie), T may be selected from:

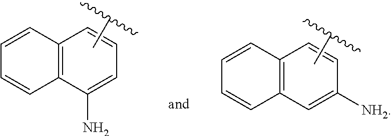

In certain embodiments, for a compound or salt of any of Formulas (B-I), (B-Ia), (B-Ib), (B-Ic), (B-Id), and (B-Ie), T is selected from:

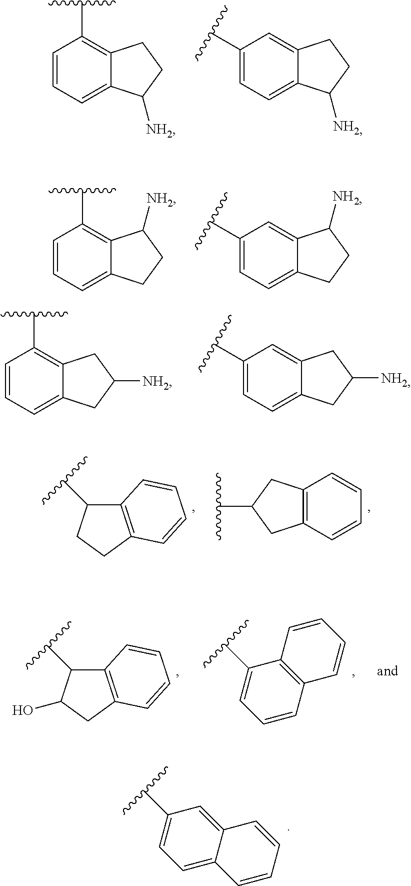

In some embodiments, for a compound or salt of any of Formulas (B-I), (B-Ia), (B-Ib), (B-Ic), (B-Id), and (B-Ie), T is selected from naphthalene, 1,2,3,4-tetrahydronaphthalene, decahydronaphthalene, octahydro-1H-indene, 2,3-dihydro-1H-indene, 1H-indene, octahydropentalene, decahydro-1H-benzo[7]annulene, 7H-benzo[7]annulene, 4aH-benzo[7]annulene, 6,7,8,9-tetrahydro-5H-benzo[7]annulene, 2,3,4,5-tetrahydro-1H-benzo[7]annulene, 2,3,4,7-tetrahydro-1H-benzo[7]annulene, azulene, and decahydroazulene, and wherein T is optionally substituted with one or more substituents independently selected at each occurrence from R¹³.

In some embodiments, for a compound or salt of any of Formulas (B-I), (B-Ia), (B-Ib), (B-Ic), (B-Id), and (B-Ie), T is selected from a 4- to 12-membered heterocycle optionally substituted with one or more substituents independently selected at each occurrence from R¹³. In certain embodiments, for a compound or salt of any of Formulas (B-I), (B-Ia), (B-Ib), (B-Ic), (B-Id), and (B-Ie), T is selected from:

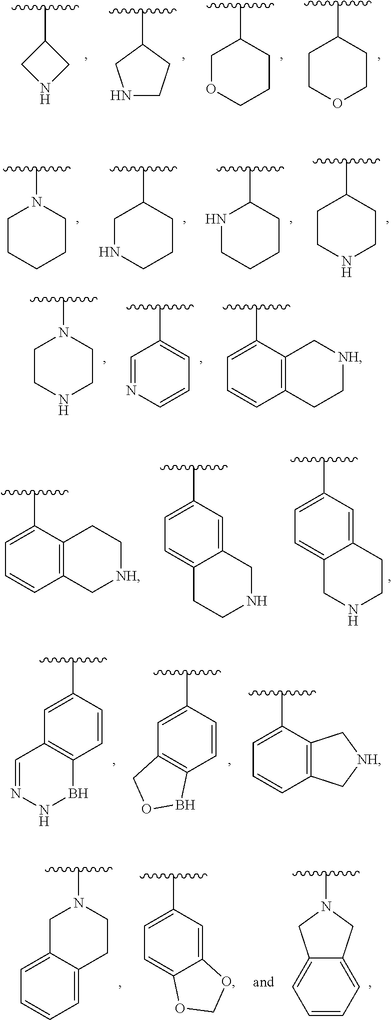

any one of which is optionally substituted with one or more substituents independently selected at each occurrence from R¹³. In certain embodiments, for a compound or salt of any of Formulas (B-I), (B-Ia), (B-Ib), (B-Ic), (B-Id), and (B-Ie), T is selected from:

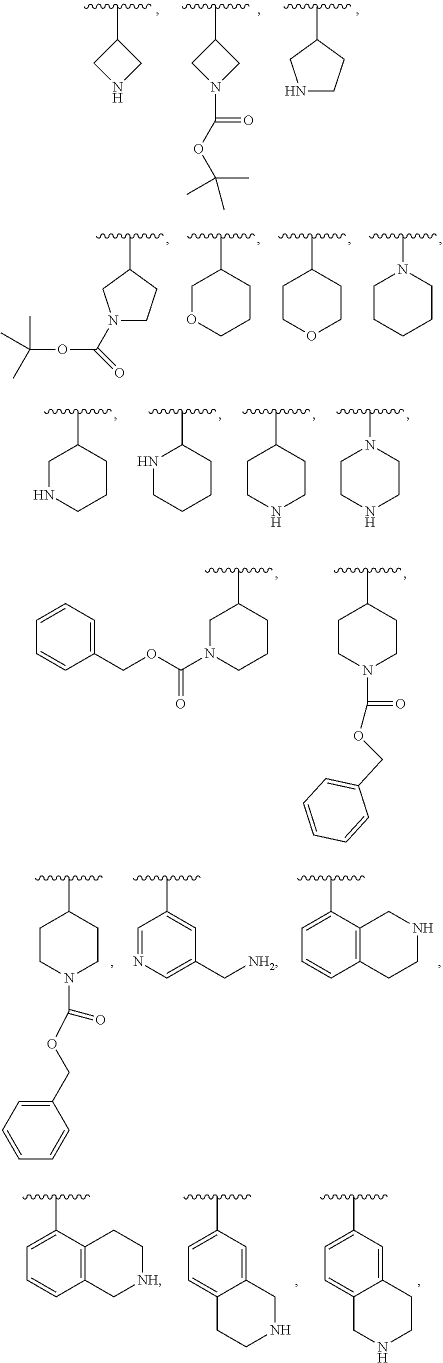

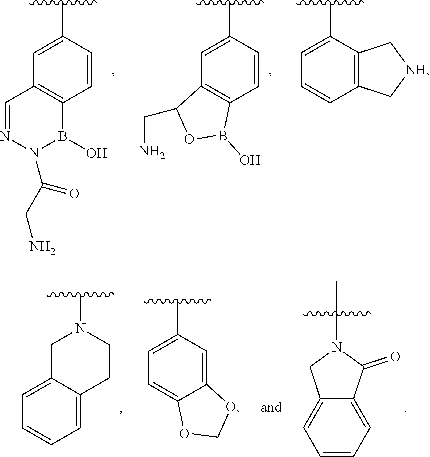

In certain embodiments, for a compound or salt of any of Formulas (B-I), (B-Ia), (B-Ib), (B-Ic), (B-Id), and (B-Ie), T is selected from a 7- to 12-membered bicyclic heterocycle optionally substituted with one or more substituents independently selected at each occurrence from R¹³. In some embodiments, for a compound or salt of any of Formulas (B-I), (B-Ia), (B-Ib), (B-Ic), (B-Id), and (B-Ie), T is selected from an 8- to 11-membered bicyclic heterocycle optionally substituted with one or more substituents independently selected at each occurrence from R¹³, such as an 8- to 11-membered bicyclic heteroaryl group.

In some embodiments, for a compound or salt of any of Formulas (B-I), (B-Ia), (B-Ib), (B-Ic), (B-Id), and (B-Ie), T is selected from:

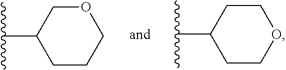

wherein T is optionally substituted with one or more substituents independently selected at each occurrence from R¹³.

In some embodiments, for a compound or salt of any one of Formulas (B-I), (B-Ia), (B-Ib), (B-Ic), (B-Id), and (B-Ie), -Q-T is selected from:

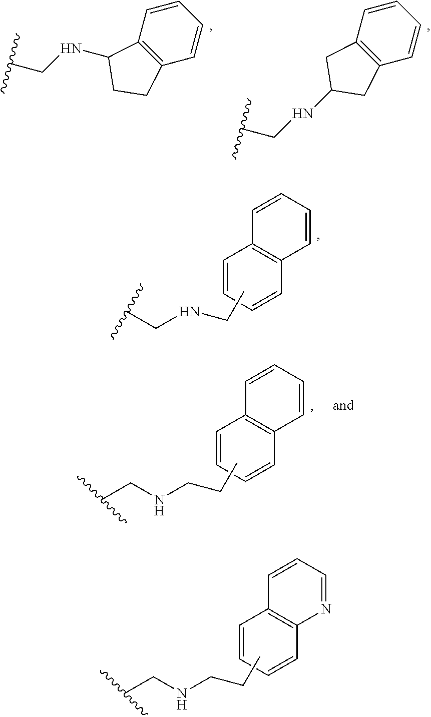

wherein T is optionally substituted with one or more substituents independently selected at each occurrence from R¹³.

In some embodiments, -Q-T is selected from

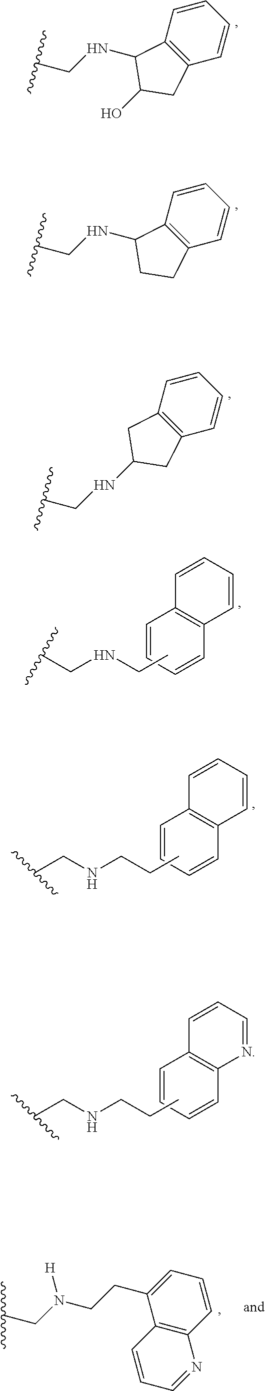

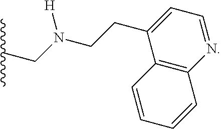 [SMILES: *CNCCc1ccnc2ccccc12]

In some embodiments, for a compound or salt of any of Formulas (B-I), (B-Ia), (B-Ib), (B-Ic), (B-Id), and (B-Ie), T has one to four ring heteroatoms independently selected from N, O, S, and B. In certain embodiments, for a compound or salt of any of Formulas (B-I), (B-Ia), (B-Ib), (B-Ic), (B-Id), and (B-Ie), T has at least one ring heteroatom that is boron. For example, for a compound or salt of any of Formulas (B-I), (B-Ia), (B-Ib), (B-Ic), (B-Id), and (B-Ie), T may be selected from:

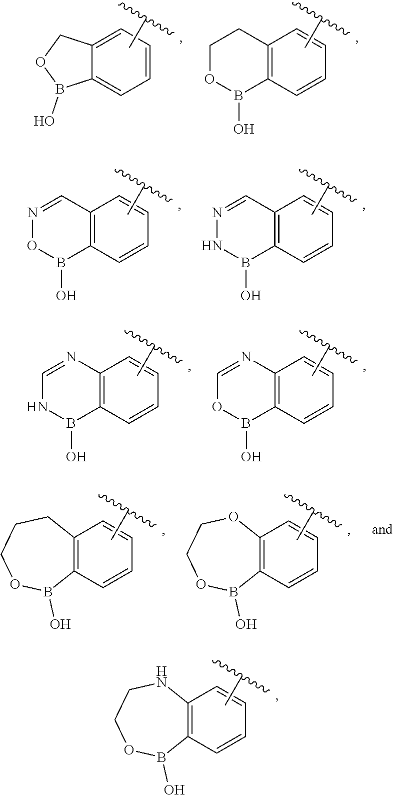

wherein T is optionally substituted with one or more substituents independently selected at each occurrence from R¹³. In certain embodiments, for a compound or salt of any of Formulas (B-I), (B-Ia), (B-Ib), (B-Ic), (B-Id), and (B-Ie), T may be selected from

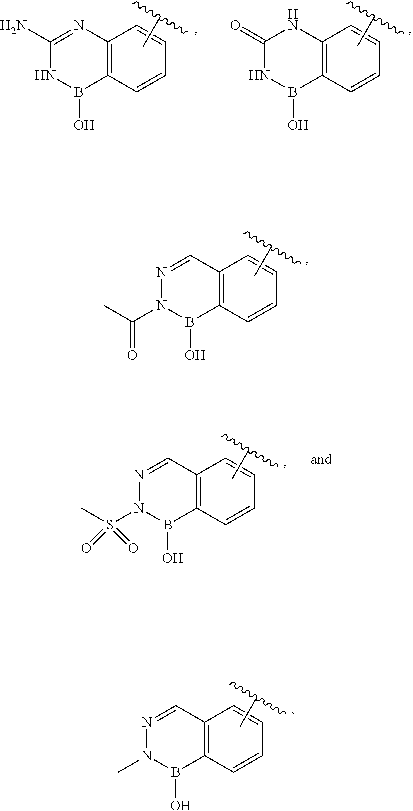

wherein T is optionally substituted with one or more substituents independently selected at each occurrence from R¹³.

In some embodiments, for a compound or salt of any of Formulas (B-I), (B-Ia), (B-Ib), (B-Ic), (B-Id), and (B-Ie), T is represented by:

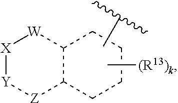

wherein dashed lines represent single or double bonds, valence permitting; k is selected from 0, 1, 2, and 3; and W, X, Y, and Z are independently selected from N(R¹⁰)_{g} and C(R¹⁰)ₕ, wherein g is selected from 0 and 1 and h is selected from 1 and 2, and wherein a straight line linked to a wavy line indicates connectivity to Q from any position, valence permitting, of the bicyclic heterocycle. In some embodiments, for a compound or salt of any of Formulas (B-I), (B-Ia), (B-Ib), (B-Ic), (B-Id), and (B-Ie), T is represented by:

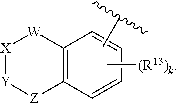

In other embodiments, for a compound or salt of any of Formulas (B-I), (B-Ia), (B-Ib), (B-Ic), (B-Id), and (B-Ie), T is represented by:

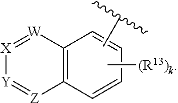

In some embodiments, for a compound or salt of any of Formulas (B-I), (B-Ia), (B-Ib), (B-Ic), (B-Id), and (B-Ie), T is selected from:

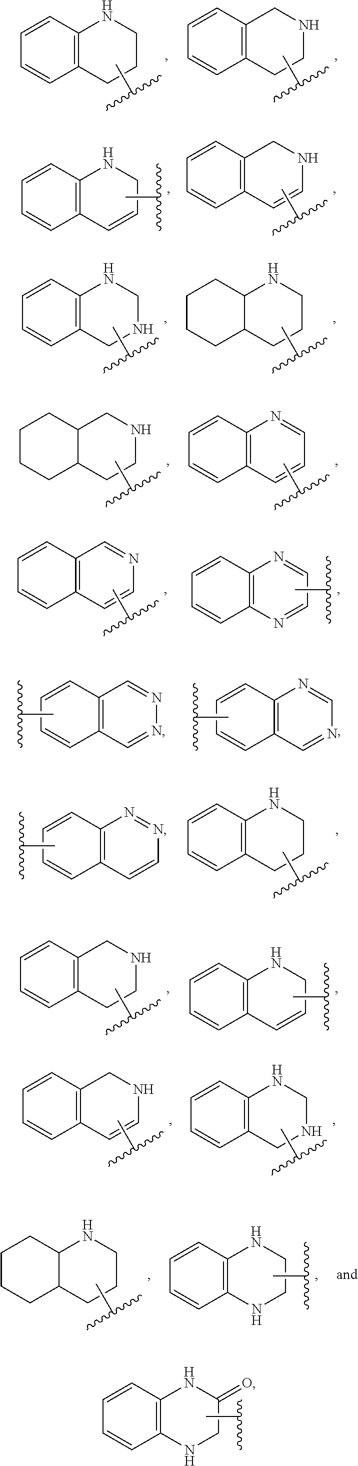

wherein T is optionally substituted with one or more substituents independently selected at each occurrence from R¹³. For example, for a compound or salt of any of Formulas (B-I), (B-Ia), (B-Ib), (B-Ic), (B-Id), and (B-Ie), T may be selected from:

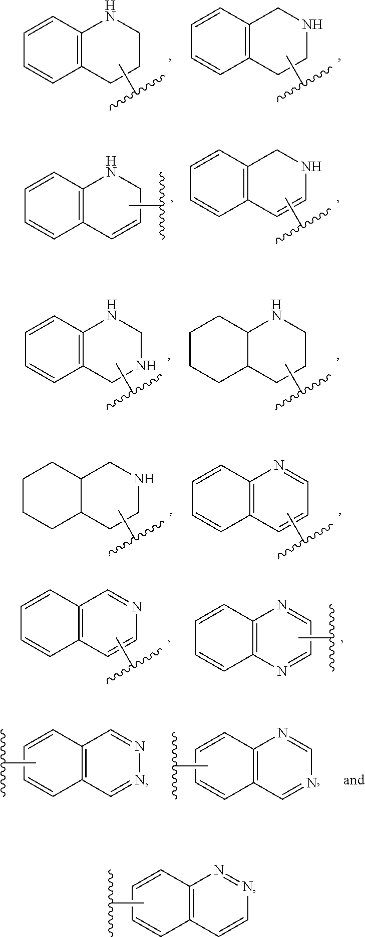

wherein T is optionally substituted with one or more substituents independently selected at each occurrence from R¹³. In certain embodiments, for a compound or salt of any of Formulas (B-I), (B-Ia), (B-Ib), (B-Ic), (B-Id), and (B-Ie), T is selected from:

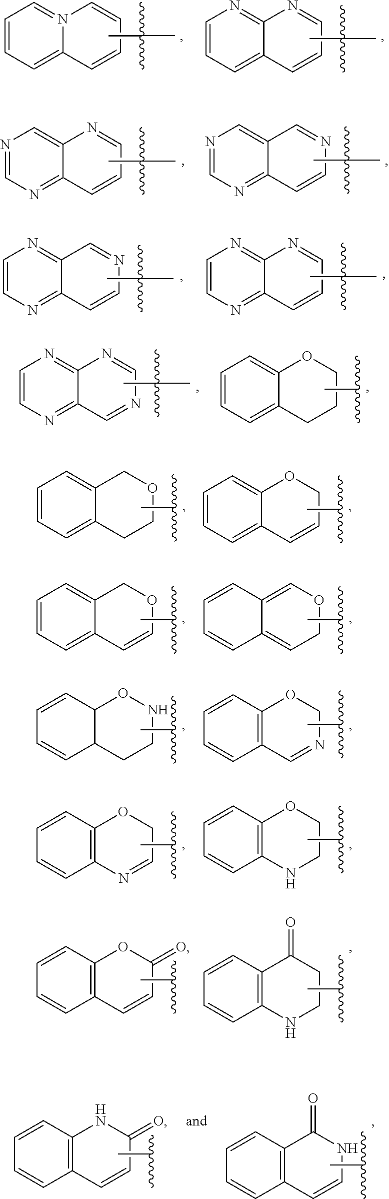

wherein Tis optionally substituted with one or more substituents independently selected at each occurrence from R¹³. In some embodiments, for a compound or salt of any of Formulas (B-I), (B-Ia), (B-Ib), (B-Ic), (B-Id), and (B-Ie), T may be selected from:

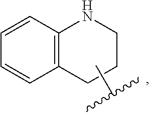

wherein T is optionally substituted with one or more substituents independently selected at each occurrence from R¹³. For example, for a compound or salt of any of Formulas (B-I), (B-Ia), (B-Ib), (B-Ic), (B-Id), and (B-Ie), T may be selected from:

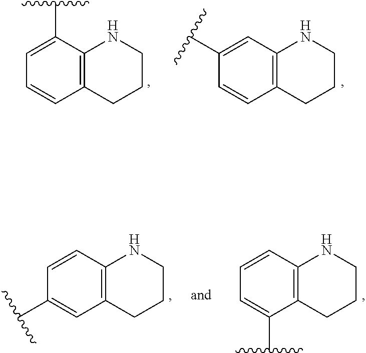

wherein T is optionally substituted with one or more substituents independently selected at each occurrence from R¹³. In some embodiments, for a compound or salt of any of Formulas (B-I), (B-Ia), (B-Ib), (B-Ic), (B-Id), and (B-Ie), T may be selected from:

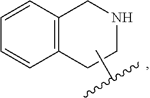

wherein T is optionally substituted with one or more substituents independently selected at each occurrence from R¹³. For example, for a compound or salt of any of Formulas (B-I), (B-Ia), (B-Ib), (B-Ic), (B-Id), and (B-Ie), T may be selected from:

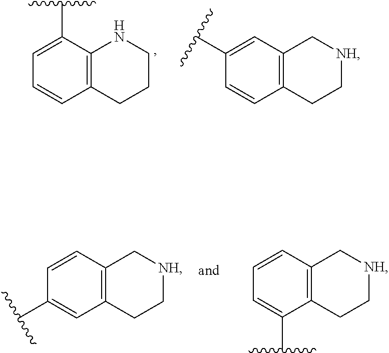

wherein T is optionally substituted with one or more substituents independently selected at each occurrence from R¹³. In some embodiments, for a compound or salt of any of Formulas (B-I), (B-Ia), (B-Ib), (B-Ic), (B-Id), and (B-Ie), T may be selected from:

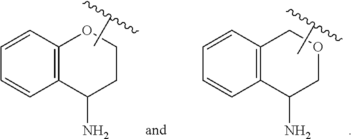

In some embodiments, for a compound or salt of any of Formulas (B-I), (B-Ia), (B-Ib), (B-Ic), (B-Id), and (B-Ie), T may be selected from:

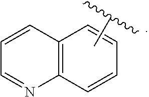

For example, for a compound or salt of any of Formulas (B-I), (B-Ia), (B-Ib), (B-Ic), (B-Id), and (B-Ie), T may be selected from:

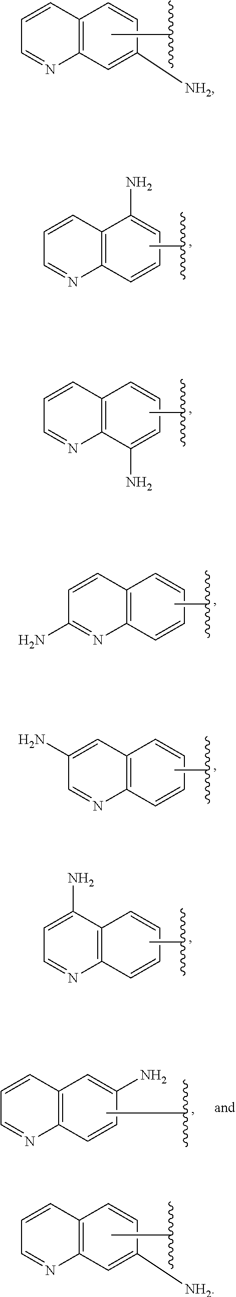

In some embodiments, for a compound or salt of any of Formulas (B-I), (B-Ia), (B-Ib), (B-Ic), (B-Id), and (B-Ie), T is selected from:

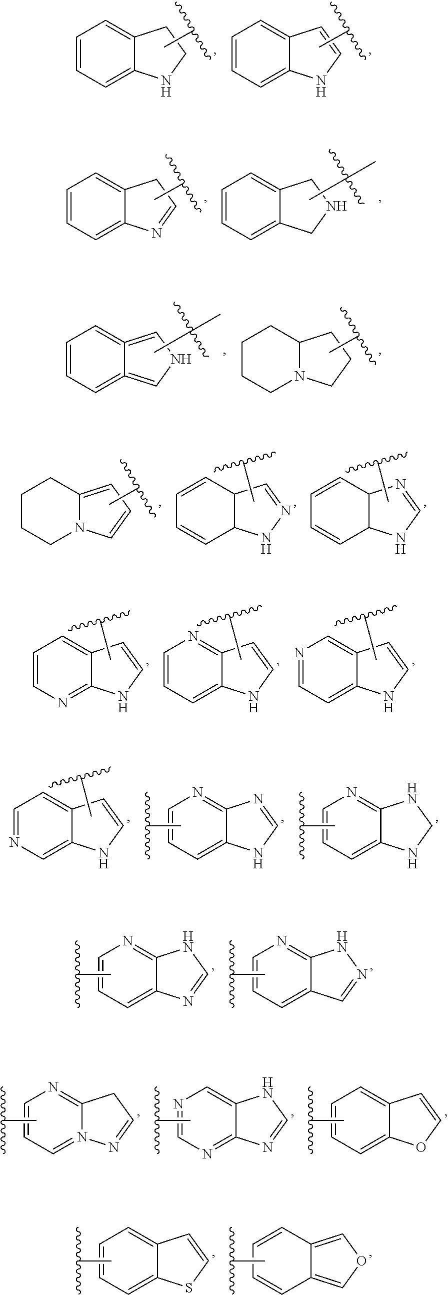

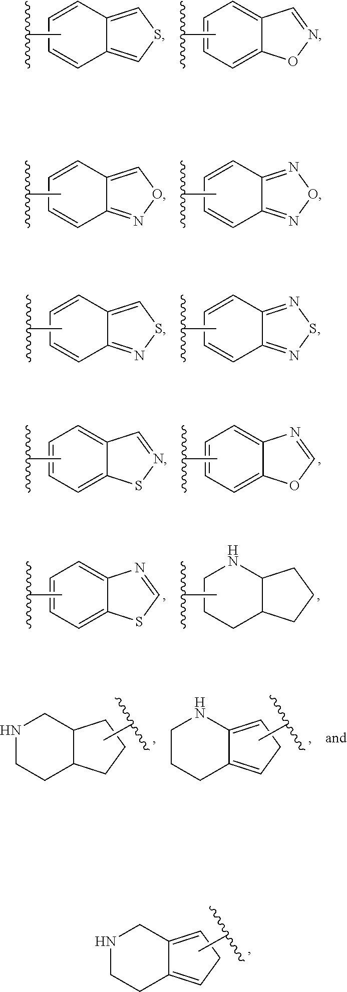

wherein T is optionally substituted with one or more substituents independently selected at each occurrence from R¹³.

In some embodiments, for a compound or salt of any of Formulas (B-I), (B-Ia), (B-Ib), (B-Ic), (B-Id), and (B-Ie), T is selected from:

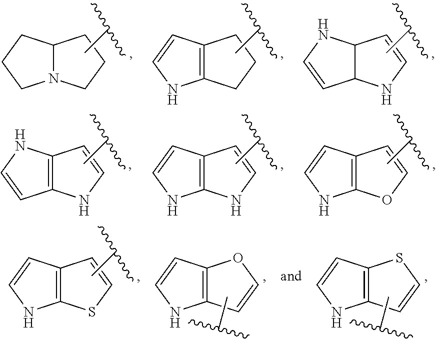

wherein T is optionally substituted with one or more substituents independently selected at each occurrence from R¹³.

In some embodiments, for a compound or salt of any of Formulas (B-I), (B-Ia), (B-Ib), (B-Ic), (B-Id), and (B-Ie), T is selected from:

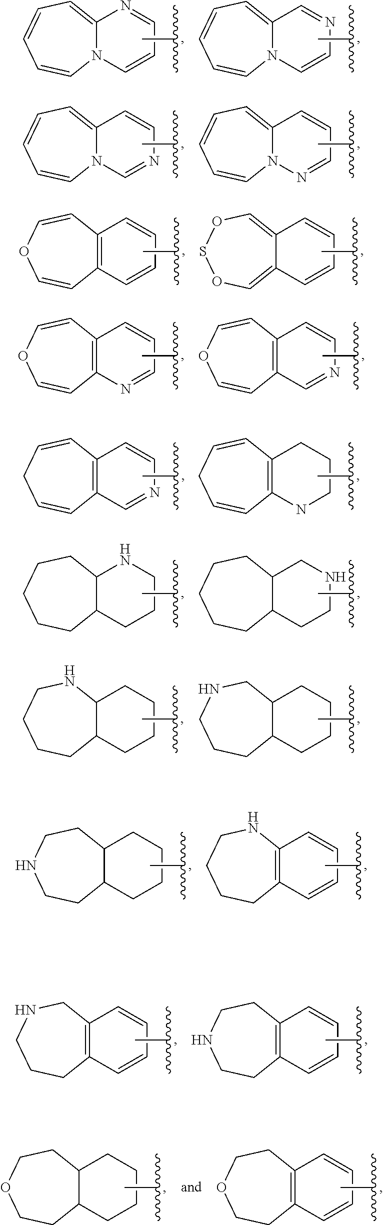

wherein T is optionally substituted with one or more substituents independently selected at each occurrence from R¹³.

In some embodiments, for a compound or salt of any of Formulas (B-I), (B-Ia), (B-Ib), (B-Ic), (B-Id), and (B-Ie), T is selected from:

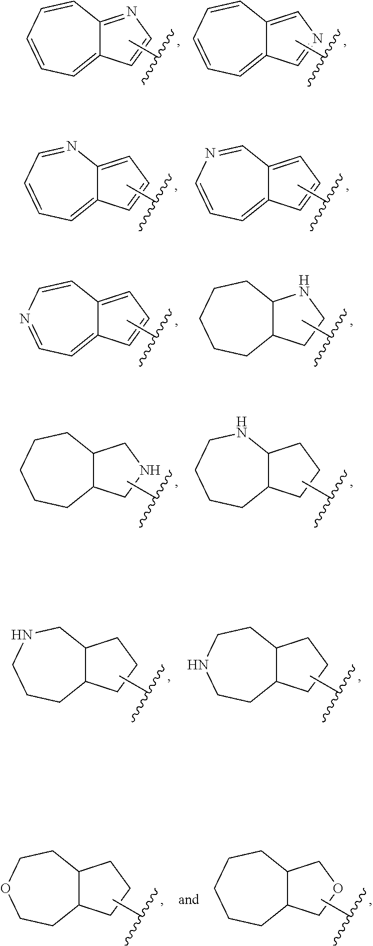

wherein T is optionally substituted with one or more substituents independently selected at each occurrence from R¹³.

In some embodiments, for a compound or salt of any of Formulas (B-I), (B-Ia), (B-Ib), (B-Ic), (B-Id), and (B-Ie), T is selected from 1,2,3,4-tetrahydroquinoline, 1,2,3,4-tetrahydroisoquinoline, 1,2-dihydroquinoline, 1,2-dihydroisoquinoline, 1,2,3,4-tetrahydroquinazoline, decahydroquinoline, decahydroisoquinoline, quinoline, isoquinoline, quinoxaline, phthalazine, quinazoline, and cinnoline, wherein T is optionally substituted with one or more substituents independently selected at each occurrence from R¹³.

In some embodiments, for a compound or salt of any of Formulas (B-I), (B-Ia), (B-Ib), (B-Ic), (B-Id), and (B-Ie), each R¹³ may be independently selected from:
a halogen, —OR¹⁰, —SR¹⁰, —N(R¹⁰)₂, —C(O)R¹⁰, —C(O)N(R¹⁰)₂—N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), and —CN; and
—C₁-C₁₀ alkyl, —C₂-C₁₀ alkenyl, and —C₂-C₁₀ alkynyl, each of which is optionally substituted at each occurrence with one or more substituents independently selected from a halogen, —OR¹⁰, —SR¹⁰, —N(R¹⁰)₂, —C(O)R¹⁰, —C(O)N(R¹⁰)₂—N(R¹⁰)C(O)R¹⁰,
   —C(O)OR¹⁰, —OC(O)R¹⁰, —S(O)R¹⁰, —S(O)₂R¹⁰, —
   S(O)₂N(R¹⁰)₂, —P(O)(OR¹⁰)₂, —OP(O)(OR¹⁰)₂, —NO₂, ═O, ═S, ═N(R¹⁰), —CN, a C₃-C₁₀ carbocycle, and a 3- to 10-membered heterocycle.

In certain embodiments, for a compound or salt of any of Formulas (B-I), (B-Ia), (B-Ib), (B-Ic), (B-Id), and (B-Ie), each R¹³ may be independently selected from a halogen, —OR¹⁰, —SR¹⁰, —N(R¹⁰)₂, —NO₂, and —CN; and —C₁-C₁₀ alkyl optionally substituted at each occurrence with one or more substituents independently selected from a halogen, —OR¹⁰, —SR¹⁰, —N(R¹⁰)₂, —C(O)R¹⁰, —NO₂, and —CN.

In certain embodiments, for a compound or salt of Formulas (B-I), (B-Ia), (B-Ib), (B-Ic), (B-Id), and (B-Ie):
Q is selected from a bond, —(CR¹⁰₂)ₚ—, and —(CR¹⁰₂)_{q}NR¹⁰(CR¹⁰₂)_{q}—;
T is selected from an optionally substituted saturated C₃-C₇ carbocycle, an optionally substituted C₅₋₁₂ bicyclic carbocycle, and an optionally substituted 4- to 12-membered heterocycle, wherein T is optionally substituted with one or more substituents independently selected at each occurrence from R¹³, wherein R¹³ is independently selected at each occurrence from halogen, —OR¹⁰, —N(R¹⁰)₂, —C(O)R¹⁰, —C(O)OR¹⁰, —N(R¹⁰)C(O)R¹⁰, and —C₁-C₃ alkyl optionally substituted with one or more substituents independently selected from a halogen, —OR¹⁰ and —N(R¹⁰)₂; and
R¹⁰ is as set forth herein (and in some aspects, R¹⁰ is H or C₁-C₃ alkyl).

In certain embodiments, for a compound or salt of Formulas (B-I), (B-Ia), (B-Ib), (B-Ic), (B-Id), and (B-Ie):
Q is selected from a bond, —(CR¹⁰₂)ₚ—, and —(CR¹⁰₂)_{q}NR¹⁰(CR¹⁰₂)_{q}—;
T is selected from an optionally substituted saturated C₃-C₇ carbocycle, an optionally substituted C₅₋₁₂ bicyclic carbocycle, and an optionally substituted 4- to 12-membered heterocycle, wherein T is optionally substituted with one or more substituents independently selected at each occurrence from R¹³, wherein R¹³ is independently selected at each occurrence from halogen, —OR¹⁰, —N(R¹⁰)₂, and —C₁-C₃ alkyl optionally substituted with one or more substituents independently selected from a halogen, —OR¹⁰ and —N(R¹⁰)₂; and
R¹⁰ is as set forth herein (and in some aspects, R¹⁰ is H or C₁-C₃ alkyl).

In certain embodiments, for a compound or salt of Formulas (B-I), (B-Ia), (B-Ib), (B-Ic), (B-Id), and (B-Ie):
Q is selected from a bond, —CH₂—, —CH₂NH—, —CH₂NHCH₂—, and —CH₂NHCH₂CH₂—; and
T is selected from:

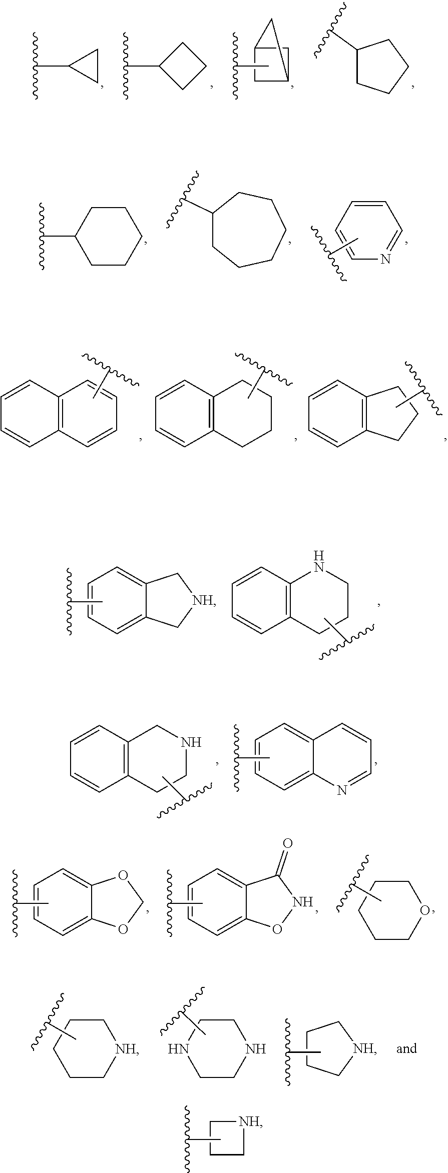

wherein T is optionally substituted with one or more substituents independently selected at each occurrence from R¹³ wherein R¹³ is independently selected at each occurrence from halogen, —OR¹⁰, —N(R¹⁰)₂, —C(O)R¹⁰, —C(O)OR¹⁰, —N(R¹⁰)C(O)R¹⁰, and —C₁-C₃ alkyl optionally substituted with one or more substituents independently selected from a halogen, —OR¹⁰ and —N(R¹⁰)₂; and
R¹⁰ is as set forth herein (and in some aspects, R¹⁰ is H or C₁-C₃ alkyl).

In certain embodiments, for a compound or salt of Formulas (B-I), (B-Ia), (B-Ib), (B-Ic), (B-Id), and (B-Ie):
Q is selected from —CH₂—, —CH₂NH—, —CH₂NHCH₂—, and —CH₂NHCH₂CH₂—; and
T is selected from:

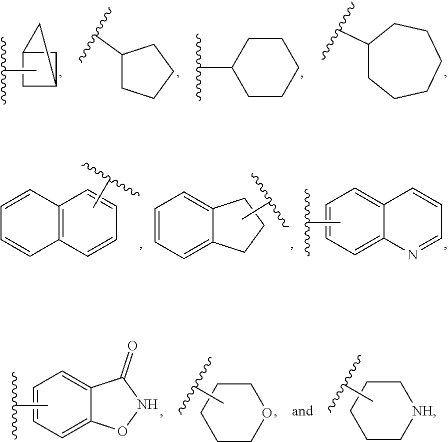

wherein T is optionally substituted with one or more substituents independently selected at each occurrence from R¹³, wherein R¹³ is independently selected at each occurrence from halogen, —OH, —NH₂, and —C₁-C₃ alkyl.

In some embodiments, the compound of Formula (B-I) is selected from:

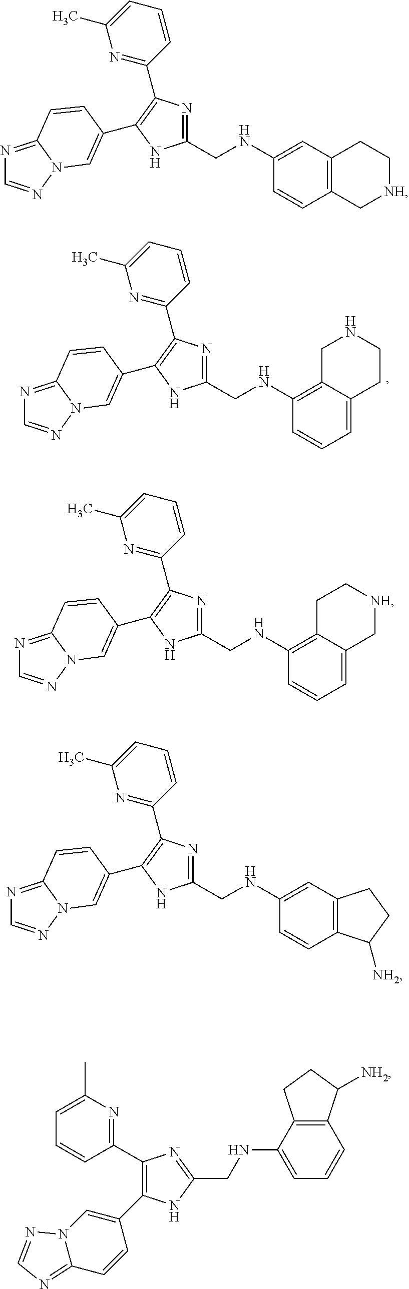 [SMILES: Cc1cccc(-c2nc(CNc3ccc4c(c3)CCNC4)[nH]c2-c2ccc3ncnn3c2)n1 ; Cc1cccc(-c2nc(CNc3cccc4c3CNCC4)[nH]c2-c2ccc3ncnn3c2)n1 ; Cc1cccc(-c2nc(CNc3cccc4c3CCNC4)[nH]c2-c2ccc3ncnn3c2)n1 ; Cc1cccc(-c2nc(CNc3ccc4c(c3)CCC4N)[nH]c2-c2ccc3ncnn3c2)n1 ; Cc1cccc(-c2nc(CNc3cccc4c3CCC4N)[nH]c2-c2ccc3ncnn3c2)n1]

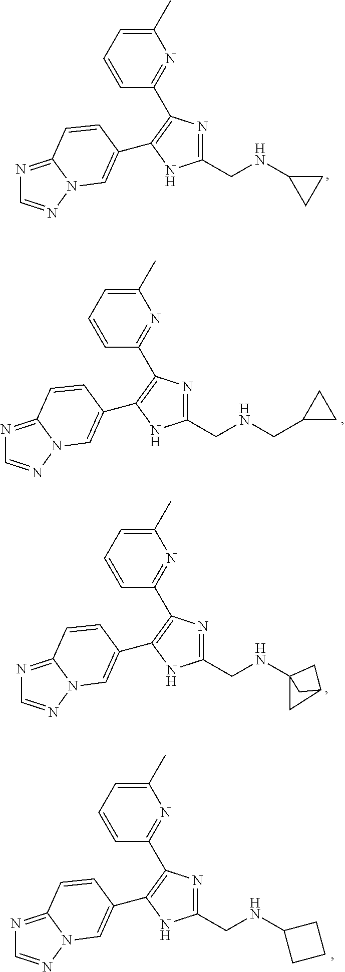 [SMILES: Cc1cccc(-c2nc(CNC3CC3)[nH]c2-c2ccc3ncnn3c2)n1 ; Cc1cccc(-c2nc(CNCC3CC3)[nH]c2-c2ccc3ncnn3c2)n1 ; Cc1cccc(-c2nc(CNC34CC(C3)C4)[nH]c2-c2ccc3ncnn3c2)n1 ; Cc1cccc(-c2nc(CNC3CCC3)[nH]c2-c2ccc3ncnn3c2)n1]

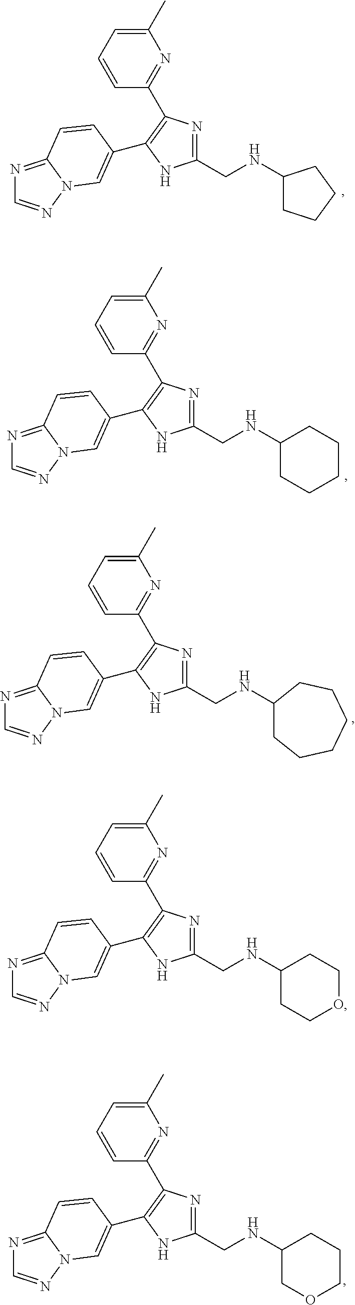 [SMILES: Cc1cccc(-c2nc(CNC3CCCC3)[nH]c2-c2ccc3ncnn3c2)n1 ; Cc1cccc(-c2nc(CNC3CCCCC3)[nH]c2-c2ccc3ncnn3c2)n1 ; Cc1cccc(-c2nc(CNC3CCCCCC3)[nH]c2-c2ccc3ncnn3c2)n1 ; Cc1cccc(-c2nc(CNC3CCOCC3)[nH]c2-c2ccc3ncnn3c2)n1 ; Cc1cccc(-c2nc(CNC3CCCOC3)[nH]c2-c2ccc3ncnn3c2)n1]

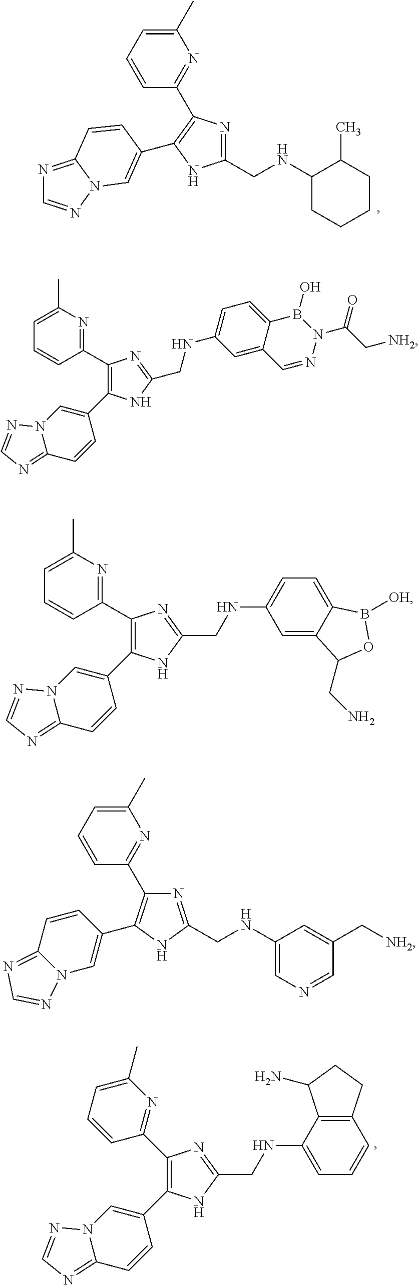 [SMILES: Cc1cccc(-c2nc(CNC3CCCCC3C)[nH]c2-c2ccc3ncnn3c2)n1 ; Cc1cccc(-c2nc(CNc3ccc4c(c3)C=NN(C(=O)CN)B4O)[nH]c2-c2ccc3ncnn3c2)n1 ; Cc1cccc(-c2nc(CNc3ccc4c(c3)C(CN)OB4O)[nH]c2-c2ccc3ncnn3c2)n1 ; Cc1cccc(-c2nc(CNc3cncc(CN)c3)[nH]c2-c2ccc3ncnn3c2)n1 ; Cc1cccc(-c2nc(CNc3cccc4c3C(N)CC4)[nH]c2-c2ccc3ncnn3c2)n1]

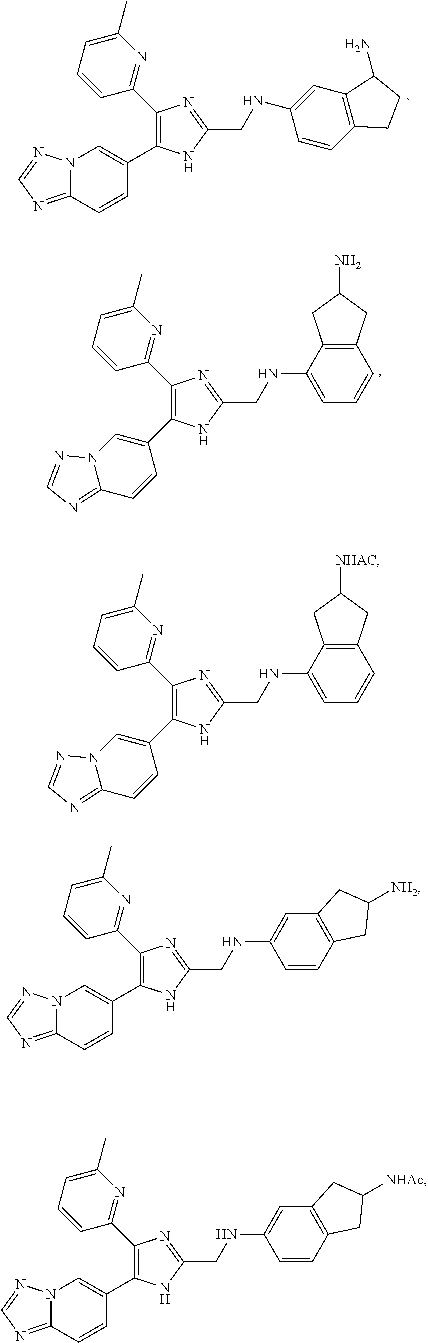

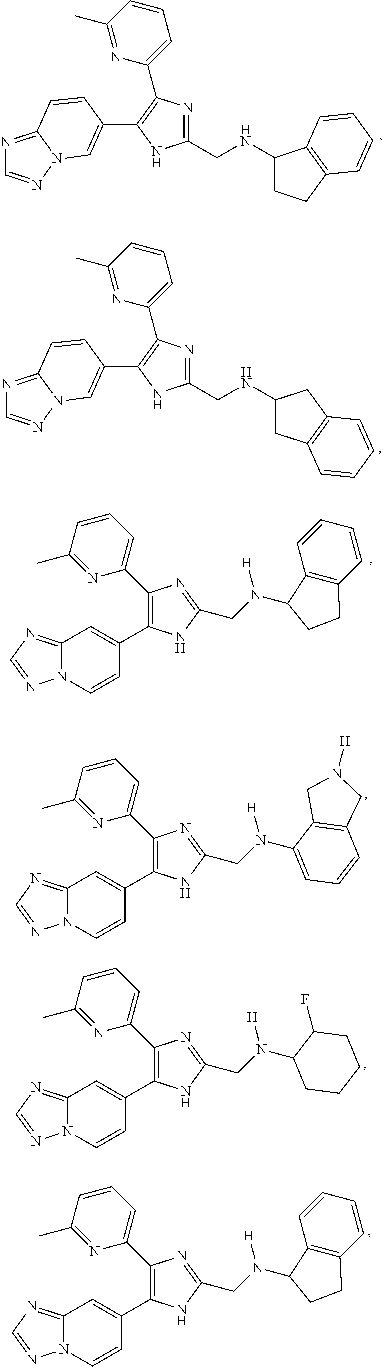

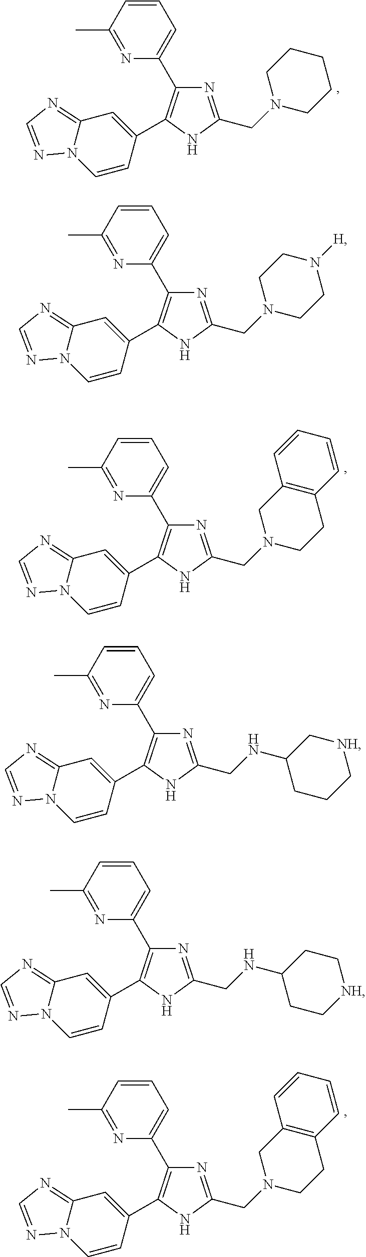

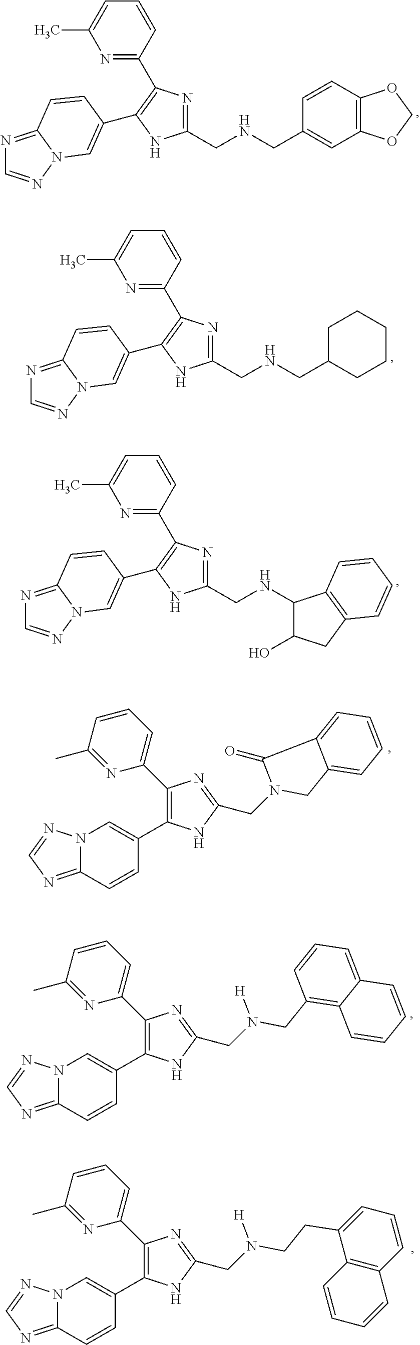 [SMILES: Cc1cccc(-c2nc(CNCc3ccc4c(c3)OCO4)[nH]c2-c2ccc3ncnn3c2)n1 ; Cc1cccc(-c2nc(CNCC3CCCCC3)[nH]c2-c2ccc3ncnn3c2)n1 ; Cc1cccc(-c2nc(CNC3c4ccccc4CC3O)[nH]c2-c2ccc3ncnn3c2)n1 ; Cc1cccc(-c2nc(CN3Cc4ccccc4C3=O)[nH]c2-c2ccc3ncnn3c2)n1 ; Cc1cccc(-c2nc(CNCc3cccc4ccccc34)[nH]c2-c2ccc3ncnn3c2)n1 ; Cc1cccc(-c2nc(CNCCc3cccc4ccccc34)[nH]c2-c2ccc3ncnn3c2)n1]

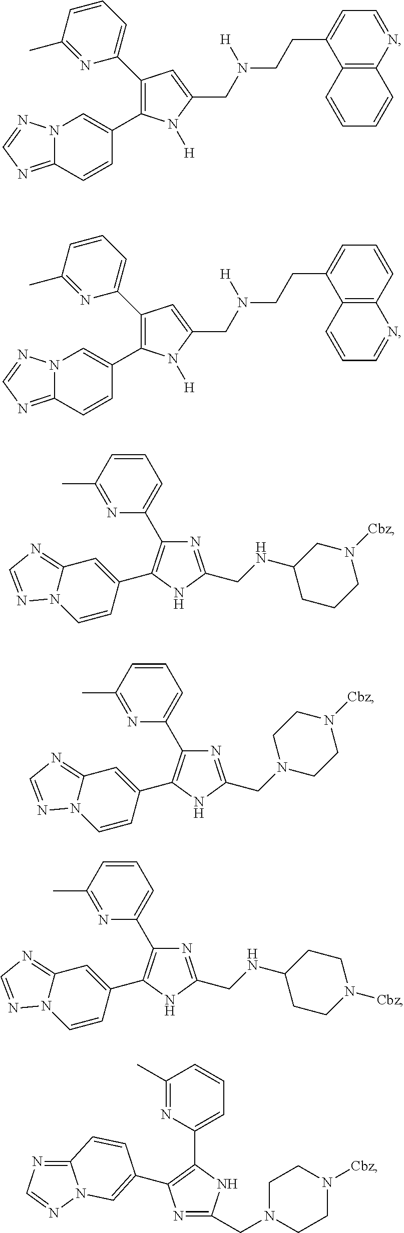

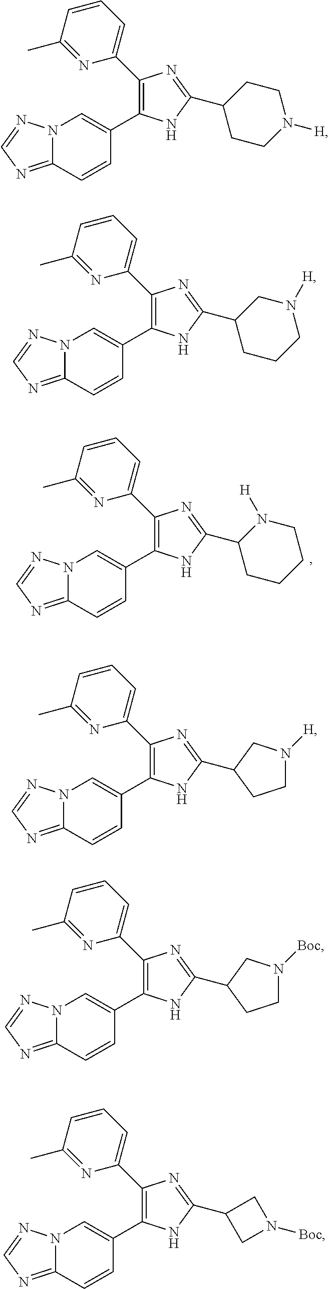

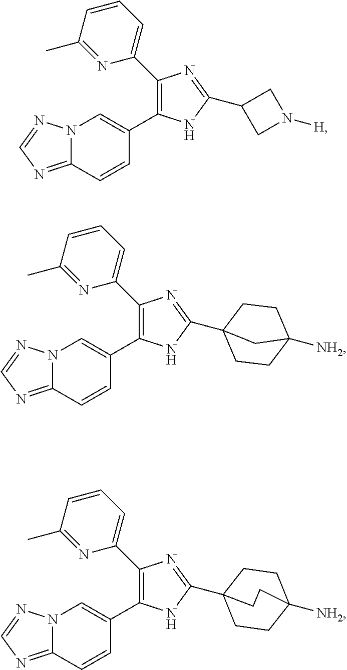

and a salt of any one thereof.

In some embodiments, the compound of Formula (B-I) is selected from:

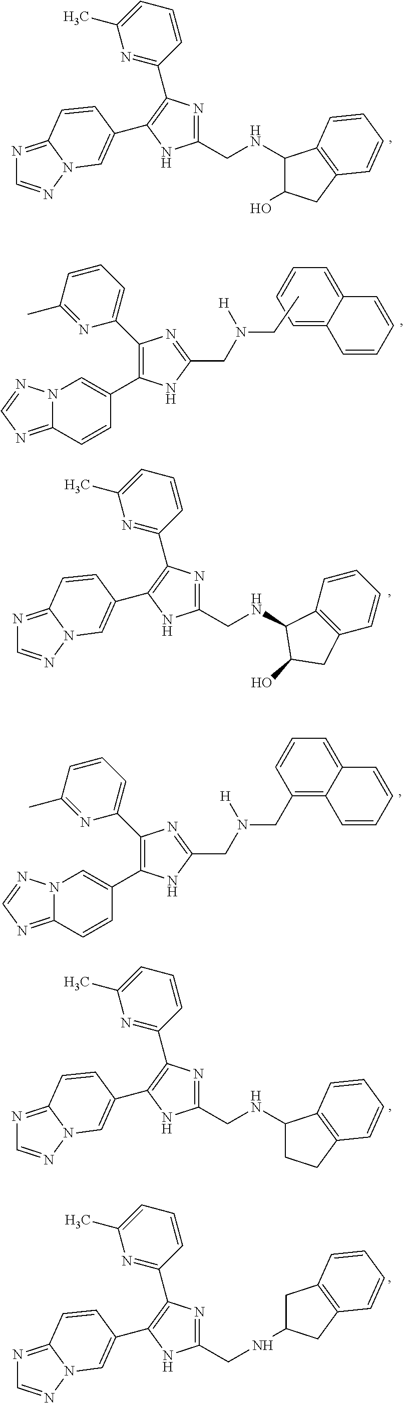

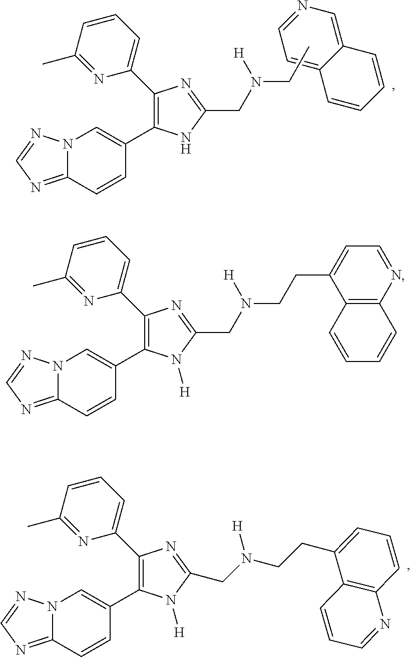

and a salt of any one thereof.

In some embodiments, the compound of Formula (B-I) is selected from the compounds in Table 4.

### Conjugates

In various aspects, a conjugate comprises:

(a) an antibody or antibody construct comprising:
(i) an antigen binding domain that specifically binds to a first antigen on a liver cell, wherein the first antigen is a liver cell antigen selected from ASGR1 or ASGR2; and
(ii) an Fc domain covalently attached to the antigen binding domain;

(b) a TGFβR1 inhibitor, e.g., a compound of Formula (A-I) or Formula (B-I) as disclosed herein; and

(c) a linker covalently attached to the TGFβR1 inhibitor and to the antibody construct.

In some aspects, the conjugate is represented by Formula (I):

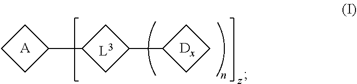

wherein: A is the ASGR1 or ASGR2 antibody or antibody construct; L³ is the linker; Dₓ is the TGFβR1 inhibitor; n is selected from 1 to 20; and z is selected from 1 to 20.

In a conjugate, the drug loading is represented by the variable z. The variable z represents the number of TGFβR1 inhibitor-linker molecules per antibody construct, or, when the variable n is equal to 1, the number of TGFβR1 inhibitors per antibody construct. Depending on the context, z can represent the average number of TGFβR1 inhibitor-linker molecules per antibody construct, also referred to the average drug loading. The variable z can range from 1 to 20, from 1-50 or from 1-100. In some conjugates, z is preferably from 1 to 8. In some preferred embodiments, when p represents the average drug loading, z ranges from about 2 to about 5. In some embodiments, z is about 2, about 3, about 4, or about 5. The average number of TGFβR1 inhibitors per antibody construct in a preparation may be characterized by conventional means such as mass spectroscopy, HIC, ELISA assay, and HPLC.

A conjugate can comprise an antibody construct with a wild-type Fc domain. A conjugate can comprise an antibody construct with a Fc domain variant. A conjugate can comprise an antibody construct with a Fc domain variant that increases the binding of the Fc domain to an Fc receptor. The Fc domain variant can comprise a substitution at more than one amino acid residue such as at 5 different amino acid residues including L235V/F243L/R292P/Y300L/P396L, as at 2 different amino acid residues including S239D/I332E, or as at 3 different amino acid residues including S298A/E333A/K334A as compared to a wild-type IgG1 Fc domain. The numbering of amino acids residues described herein is according to the EU index.

In some aspects, in addition to a first antigen binding domain specific for ASGR1 or ASGR2, the antibody construct can further comprise a second antigen binding domain that specifically binds to a second antigen on the liver cell, wherein the second antigen can be a second liver cell antigen. In some aspects, the second antigen binding domain is covalently attached to the antibody construct at a C-terminal end of the Fc domain. In some aspects, the second antigen binding domain is covalently attached to a C-terminal end of a light chain of the antibody construct. In some aspects, the second liver antigen is expressed on a canalicular cell, Kupffer cell, hepatocyte, or a combination thereof. In some aspects, the second liver antigen is a hepatocyte antigen. In some aspects, the second liver cell antigen is selected from ASGR1, ASGR2, TRF2, UGT1A1, SLC22A7, SLC13A5, SLC22A1, and C9. In some aspects, the second liver cell antigen is selected from ASGR1, ASGR2, and TRF2. In some aspects, the first antigen is different than the second antigen.

In some aspects, the Fc domain is an IgG region. In some aspects, the Fc domain is an IgG1 Fc region. In some aspects, the Fc domain is an Fc domain variant comprising one or more amino acid substitutions in an IgG region as compared to an amino acid sequence of a wild-type IgG region. In some aspects, the Fc domain variant has increased affinity to one or more Fcγ receptors as compared to the wild-type IgG region. In some aspects, the Fc domain is a non-antibody scaffold.

In some aspects, the ASGR1 or ASGR2 binding domain comprises an immunoglobulin heavy chain variable region or an antigen binding fragment thereof and an immunoglobulin light chain variable region or an antigen binding fragment thereof. In some aspects, the ASGR1 or ASGR2 binding domain comprises a single chain variable region fragment (scFv). In some aspects, a second antigen binding domain comprises an immunoglobulin heavy chain variable region or an antigen binding fragment thereof and an immunoglobulin light chain variable region or an antigen binding fragment thereof. In some aspects, a second antigen binding domain comprises a single chain variable region fragment (scFv). In some aspects, the Fc domain is covalently attached to the ASGR1 or ASGR2 binding domain: (a) as an Fc domain-antigen binding domain fusion protein; or (b) by conjugation via a second linker. In some aspects, an ASGR1 or ASGR2 antibody construct has a Kd for binding of the Fc domain to an Fc receptor in the presence of the TGFβR1 inhibitor and wherein the K_{d} for binding of the Fc domain to the Fc receptor in the presence of the TGFβR1 inhibitor is minimal or non-binding, or is no greater than about 100 times a K_{d} for binding of the Fc domain to the Fc receptor in the absence of the TGFβR1 inhibitor.

In any of the aforementioned conjugates, the antibody is specific for ASGR1 and the ASGR1 antibody is any of the antibodies selected from those described and listed in the above section entitled “Anti-ASGR1 Antibodies and Binding Domains Thereof.”

The disclosure provides a method of preparing an antibody conjugate of the formula:

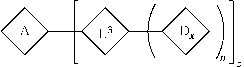

wherein:
A is an ASGR1 or ASGR2 antibody construct;
n is selected from 1 to 20;
L³ is a linker; and
Dₓ is a compound of Formula (A-I) or Formula (B-I);

comprising contacting D-L³ with an antibody construct (A).

Synthetic chemistry transformations and methodologies useful in synthesizing the compounds and conjugates described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Linker Region/Linker L³

The conjugates of the present disclosure include a linker region, such as L³, that attaches an antibody construct to at least one TGFβR1 inhibitor. In the conjugates, an inhibitor as described herein is covalently bound to an antibody construct by way of a linker, such as, for example, L³. A linker may be selected from any of the linker moieties discussed herein. The linker can be, for example, a cleavable or a non-cleavable linker. Linkers of the conjugates and methods described herein may not affect the binding of active portions of a conjugate (e.g., antigen binding domains and Fe domains) to a target or an Fc receptor. A conjugate can comprise multiple linkers. These linkers can be the same linkers or different linkers.

Linkers of the disclosure (e.g., L³) may have from about 10 to about 500 atoms in a linker, such as from about 10 to about 400 atoms, such as about 10 to about 300 atoms in a linker. In certain embodiments, linkers of the disclosure have from about 30 to about 400 atoms, such as from about 30 to about 300 atoms in the linker.

In certain embodiments, an inhibitor as described herein is covalently bound to a linker. The linker may be covalently bound to any position on the inhibitor, valence permitting. The linker may comprise a reactive moiety, e.g., a nucleophile or an electrophile that can react to form a covalent bond.

As will be appreciated by skilled artisans, a linker connects a TGFβR1 inhibitor to the antibody construct of the conjugate by forming a covalent linkage to the TGFβR1 inhibitor at one location and a covalent linkage to the antibody construct of the conjugate at another location. A conjugate may be prepared by contacting an antibody construct with a linker-compound described herein under conditions in which the linker-compound covalently links to the antibody construct. One embodiment pertains to a method of making a conjugate by contacting a linker-compound with an antibody construct under conditions in which the linker-compound covalently links to the antibody construct. The covalent linkages can be formed by reaction between functional groups in synthetic precursors for each region. Thus, the covalent linkages can be formed by reaction between functional groups on the linker and functional groups on the TGFβR1 inhibitor and antibody construct.

These conjugates can be made by various methods. It is understood that one skilled in the art may be able to make these compounds by similar methods or by combining other methods known to one skilled in the art. It is also understood that one skilled in the art would be able to make, in a similar manner as described herein by using the appropriate starting materials and modifying the synthetic route as needed. Starting materials and reagents can be obtained from commercial vendors or synthesized according to sources known to those skilled in the art or prepared as described herein.

One embodiment pertains to a conjugate formed by contacting an antibody construct that specifically binds to a liver antigen(s), a viral antigen(s) expressed on a liver cell, or both, with a linker described herein under conditions in which the linker covalently links to the antibody construct. One embodiment pertains to a method of making a conjugate formed by contacting a linker described herein under conditions in which the linker covalently links to an antibody construct.

Linkers can be short, long, flexible, rigid, cleavable, non-cleavable, hydrophilic, hydrophobic, unbranched (e.g., where z is 1), or branched (e.g., where z is greater than 1). A linker can contain connector segments that have different characteristics, such as segments of flexibility and segments of rigidity, or segments that are cleavable and segments that are cleavable. A linker can contain multiple segments, such as one or more non-cleavable segments and one or more cleavable segments. The linkers may be polyvalent such that they covalently link more than one inhibitor to a single site on an antibody construct, or monovalent such that covalently they link a single inhibitor to a single site on the antibody construct. A linker can comprise alkylene, alkenylene, alkynylene, polyether, polyester, polyamide, polyamino acid, polypeptide, cleavable peptide, and/or para-aminobenzylcarbamate groups. In some embodiments, the linker comprises a “non-cleavable” segment, such as a “non-cleavable linker,” that is chemically stable to extracellular environments, for example, chemically stable in the blood stream and in intracellular environments. In some embodiments, the linker comprises a “cleavable” segment, such as a “cleavable linker,” that includes one or more linkages that are not stable, such as linkages that are designed to cleave and/or immolate or otherwise breakdown specifically or non-specifically in the blood stream and/or inside cells (i.e., in an intracellular environment). Linkers comprise one or more cleavable segments, one or more non-cleavable segments, or a combination thereof.

Cleavable linkers can be cleavable in vitro and in vivo. Cleavable linkers can include chemically or enzymatically unstable or degradable linkages. Cleavable linkers can incorporate one or more chemical bonds that are either chemically or enzymatically cleavable while the remainder of the linker can be non-cleavable. Cleavable linkers can rely on processes inside the cell to liberate a TGFβR1 inhibitor, such as reduction in the cytoplasm, exposure to acidic conditions in the lysosome, or cleavage by specific proteases or other enzymes within the cell. A cleavable linker can be sensitive to (i.e., cleavable by) enzymes at a specific site. A cleavable linker can be cleaved by enzymes such as proteases.

A linker can contain a chemically labile group such as hydrazone and/or disulfide groups. Linkers comprising chemically labile groups can exploit differential properties between the plasma and some cytoplasmic compartments. The intracellular conditions that can facilitate TGFβR1 inhibitor release for hydrazone containing linkers can be the acidic environment of endosomes and lysosomes, while the disulfide containing linkers can be reduced in the cytosol, which can contain high thiol concentrations, e.g., glutathione. The plasma stability of a linker containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis once the conjugate is internalized into mildly acidic endosomal (pH 5.0-6.5) and lysosomal (pH 4.5-5.0) compartments of the cell. This pH dependent release mechanism can be associated with nonspecific release of the drug. To increase the stability of the hydrazone group of the linker, the linker can be varied by chemical modification, e.g., substitution, allowing tuning to achieve more efficient release in the lysosome with a minimized loss in circulation.

Hydrazone-containing linkers can contain additional cleavage sites, such as additional acid-labile cleavage sites and/or enzymatically labile cleavage sites. Conjugates including exemplary hydrazone-containing linkers can include, for example, the following structures:

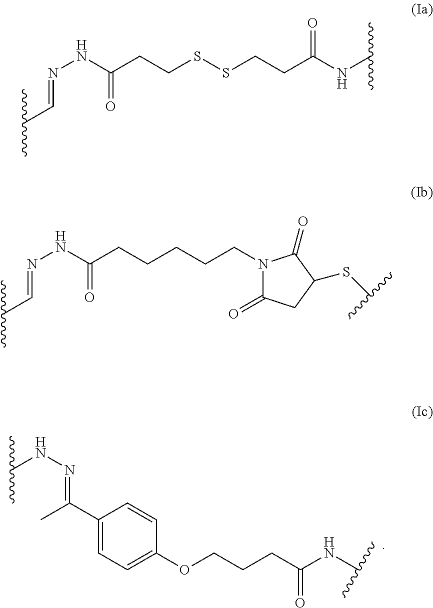 [SMILES: */C=N/NC(=O)CCSSCCC(=O)N* ; */C=N/NC(=O)CCCCCN1C(=O)CC(S*)C1=O ; *N/N=C(\C)c1ccc(OCCCC(=O)N*)cc1]

In certain linkers, such as linker (Ia), the linker can comprise two cleavable groups—a disulfide and a hydrazone moiety. For such linkers, effective cleavage can require acidic pH or disulfide reduction and acidic pH. Linkers such as (Ib) and (Ic) can be effective with a single hydrazone cleavage site.

Other acid-labile groups that can be included in linkers include cis-aconityl-containing linkers. cis-Aconityl chemistry can use a carboxylic acid juxtaposed to an amide bond to accelerate amide hydrolysis under acidic conditions.

Cleavable linkers can also include a disulfide group. Disulfides can be thermodynamically stable at physiological pH and can be designed to release upon internalization inside cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing linkers can be reasonably stable in circulation, selectively releasing the TGFβR1 inhibitor in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing linker can be enhanced by chemical modification of the linker, e.g., use of steric hindrance adjacent to the disulfide bond.

Exemplary cleavable linkers including disulfide moieties can include the following structures:

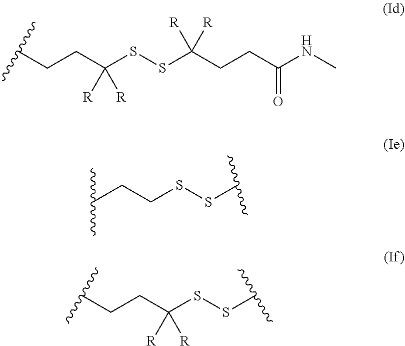

wherein R is independently selected at each occurrence from hydrogen or alkyl, for example. Increasing steric hindrance adjacent to the disulfide bond can increase the stability of the linker. Structures such as (Id) and (If) can show increased in vivo stability when one or more R groups is selected from a lower alkyl such as methyl.

Another type of cleavable linker is specifically cleaved by an enzyme. For example, the linker can be cleaved by a lysosomal enzyme. Such linkers can be peptide-based or can include peptidic regions that can act as substrates for enzymes. Peptide-based linkers can be more stable in plasma and extracellular milieu than chemically labile linkers.

Peptide bonds can have good serum stability, as lysosomal proteolytic enzymes can have very low activity in blood due to endogenous inhibitors and the unfavorably high pH value of blood compared to lysosomes. Release of a TGFβR1 inhibitor from a conjugate can occur due to the action of lysosomal proteases, e.g., cathepsin and/or plasmin. These proteases can be present at elevated levels in certain tumor tissues. The linker can be cleavable by a lysosomal enzyme. The lysosomal enzyme can be, for example, cathepsin B, β-glucuronidase, or β-galactosidase.

In a linker, a cleavable peptide can be selected from tetrapeptides or dipeptides such as Val-Cit, Val-Ala, and Phe-Lys. Dipeptides can have lower hydrophobicity compared to longer peptides, depending on the composition of the peptide. A variety of dipeptide-based cleavable linkers can be used in the conjugates described herein.

In some embodiments, the cleavable linker comprises a cleavable peptide. In certain embodiments, a peptide can be selected to contain natural amino acids, unnatural amino acids, or any combination thereof. In some embodiments, the cleavable peptide is a dipeptide, tripeptide, or tetrapeptide. In some embodiments, the cleavable peptide is Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; Trp-Cit; Ala-Ala-Asn; Gly-Phe-Leu-Gly; Gly-Gly-Phe-Gly; or Ala-Leu-Ala-Leu.

In some embodiments, the cleavable linker comprises a structure of formula:

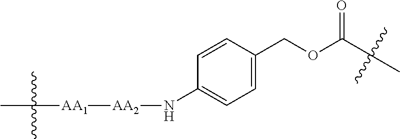

wherein -AA₁-AA₂- is the cleavable dipeptide and AA₁ and AA₂ are each an amino acid.

In some embodiments, the cleavable dipeptide is Val-Cit.

Enzymatically cleavable linkers can include a self-immolative spacer to spatially separate the TGFβR1 inhibitor from the site of enzymatic cleavage. The direct attachment of a TGFβR1 inhibitor to a peptide linker can result in proteolytic release of an amino acid adduct of the TGFβR1 inhibitor, thereby impairing its activity. The use of a self-immolative spacer can allow for the elimination of the fully active, chemically unmodified TGFβR1 inhibitor upon amide bond hydrolysis.

One self-immolative spacer can be a bifunctional para-aminobenzyl alcohol group, which can link to the peptide through the amino group, forming an amide bond, while amine containing TGFβR1 inhibitors can be attached through carbamate functionalities to the benzylic hydroxyl group of the linker to form a para-aminobenzyl carbamate (PMBC) group. The resulting pro-TGFβR1 inhibitor can be activated upon protease-mediated cleavage, leading to a 1,6-elimination reaction releasing the unmodified TGFβR1 inhibitor, carbon dioxide, and remnants of the linker group. The following scheme depicts the fragmentation of a substituted p-aminobenzyl carbamate and release of the compound:

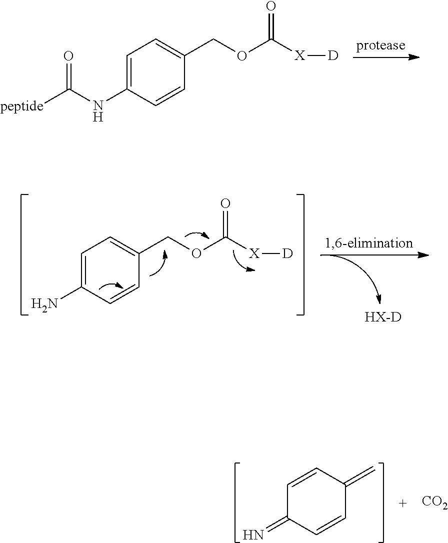

wherein HX-D represents the unmodified inhibitor.

In some embodiments, a cleavable linker comprises a valine-citrulline peptide or valine-alanine peptide. A cleavable linker can be a valine-citrulline peptide or a valine-alanine peptide. A valine-citrulline or valine-alanine-containing linker can contain a succimide group. A valine-citrulline- or valine-alanine-containing linker can contain a para-aminobenzyl (PAB) group. In some embodiments, the PAB group is a para-aminobenzyl alcohol (PABA) group or a para-aminobenzyl carbamate (PABC) group. In some embodiments, a valine-citrulline- or valine-alanine-containing linker also includes a PAB group, such as a PABC group. In some embodiments, a cleavable linker comprises a -(valine-citrulline)-(para-aminobenzyloxycarbonyl) group. In some embodiments, a cleavable linker comprises a lysine with an N-terminal amine acetylated, and a valine-citrulline cleavage site. In some embodiments, the cleavable linker comprises a succinimide group. In some embodiments, a valine-citrulline- or valine-alanine-containing linker comprises a PAB or PABC group and a succinimide group. In some embodiments, a cleavable linker is a (succinimidocaproyl)-(valine-citrulline)-(para-aminobenzyloxycarbonyl) group. In some embodiments, a cleavable linker comprises a lysine with an N-terminal amine acetylated, and a valine-citrulline cleavage site.

A linker can contain an enzymatically cleavable peptide, for example, a linker comprising structural formula (CIIIa), (CIIIb), (CIIIc), or (CIIId):

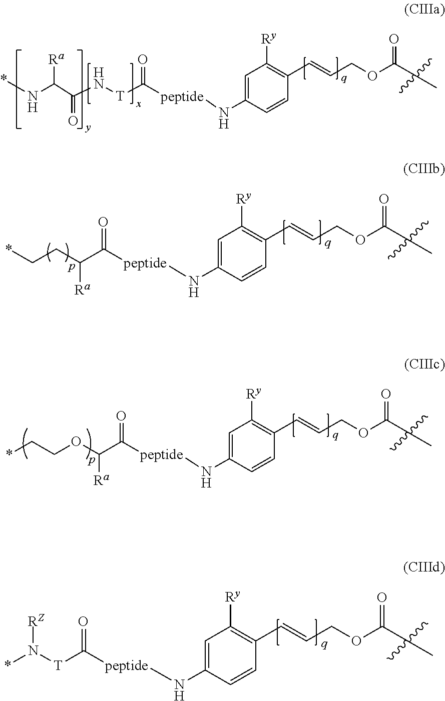

or a salt thereof, wherein: “peptide” represents a peptide (illustrated in N→C orientation, wherein peptide includes the amino and carboxy “termini”) that is cleavable by a lysosomal enzyme; T represents a polymer comprising one or more ethylene glycol units or an alkylene chain, or combinations thereof; R^{a} is selected from hydrogen, alkyl, sulfonate and methyl sulfonate; R^{y} is hydrogen or C₁₋₄ alkyl-(O)ᵣ—(C₁₋₄ alkylene)ₛ-G¹ or C₁₋₄ alkyl-(N)-[(C₁₋₄ alkylene)-G¹]₂; R^{z} is C₁₋₄ alkyl-(O)ᵣ—(C₁₋₄ alkylene)ₛ-G²; G¹ is —SO₃H, —CO₂H, PEG 4-32, or a sugar moiety; G² is —SO₃H, —CO₂H, or a PEG 4-32 moiety; r is 0 or 1; s is 0 or 1; p is an integer ranging from 0 to 5; q is 0 or 1; x is 0 or 1; y is 0 or 1; [symbol] represents the point of attachment of the linker to a compound of Formula (I), (II), (III), (IV), (V), or (VI), or pharmaceutically acceptable isomer, tautomer, racemate, hydrate, solvate, isotope, or salt thereof; and * represents the point of attachment to the remainder of the linker.

Exemplary embodiments of linkers according to structural formula (CIIIa) are illustrated below (as illustrated, the linkers include a reactive group suitable for covalently linking the linker to an antibody, an antibody construct, or a targeting moiety):

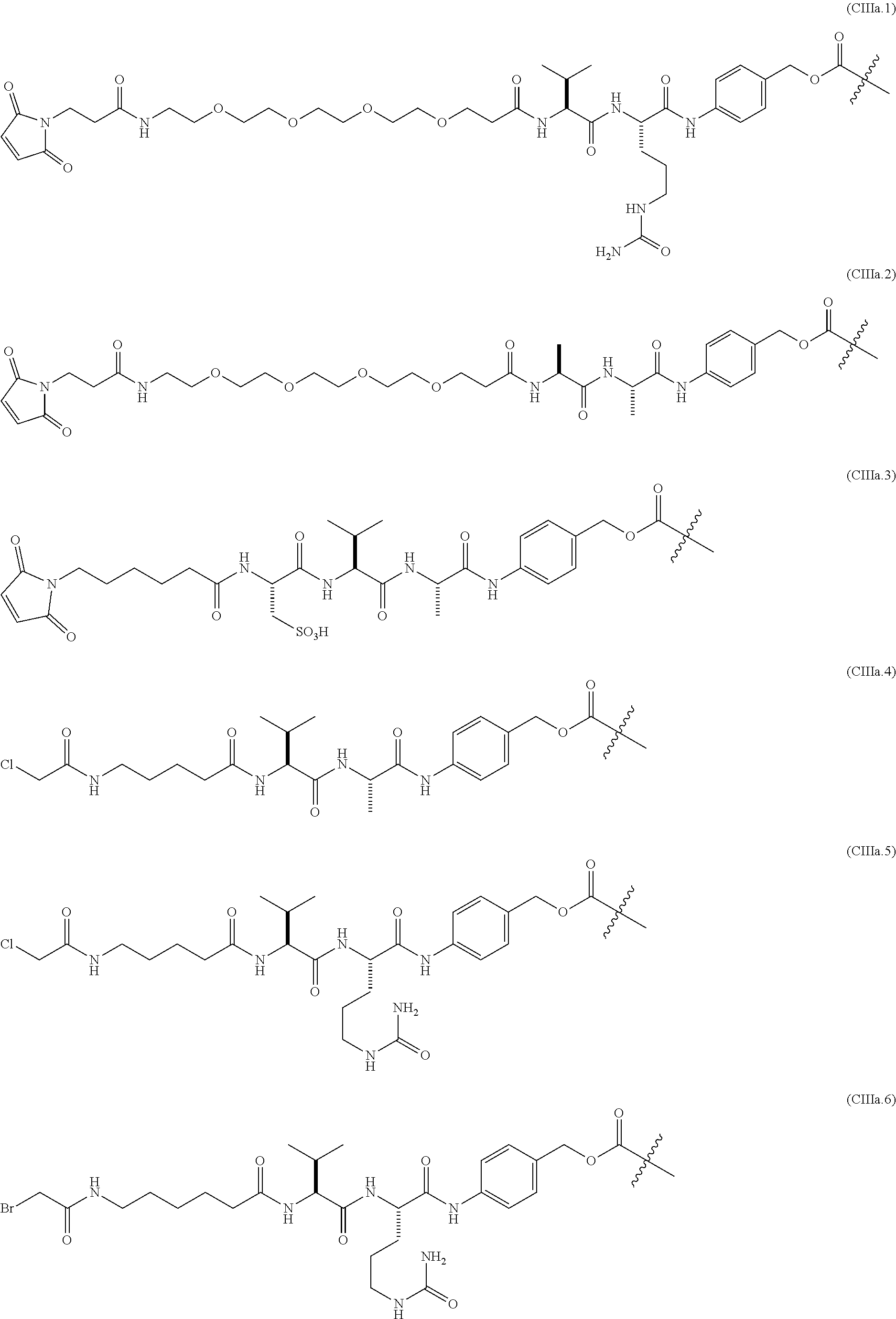

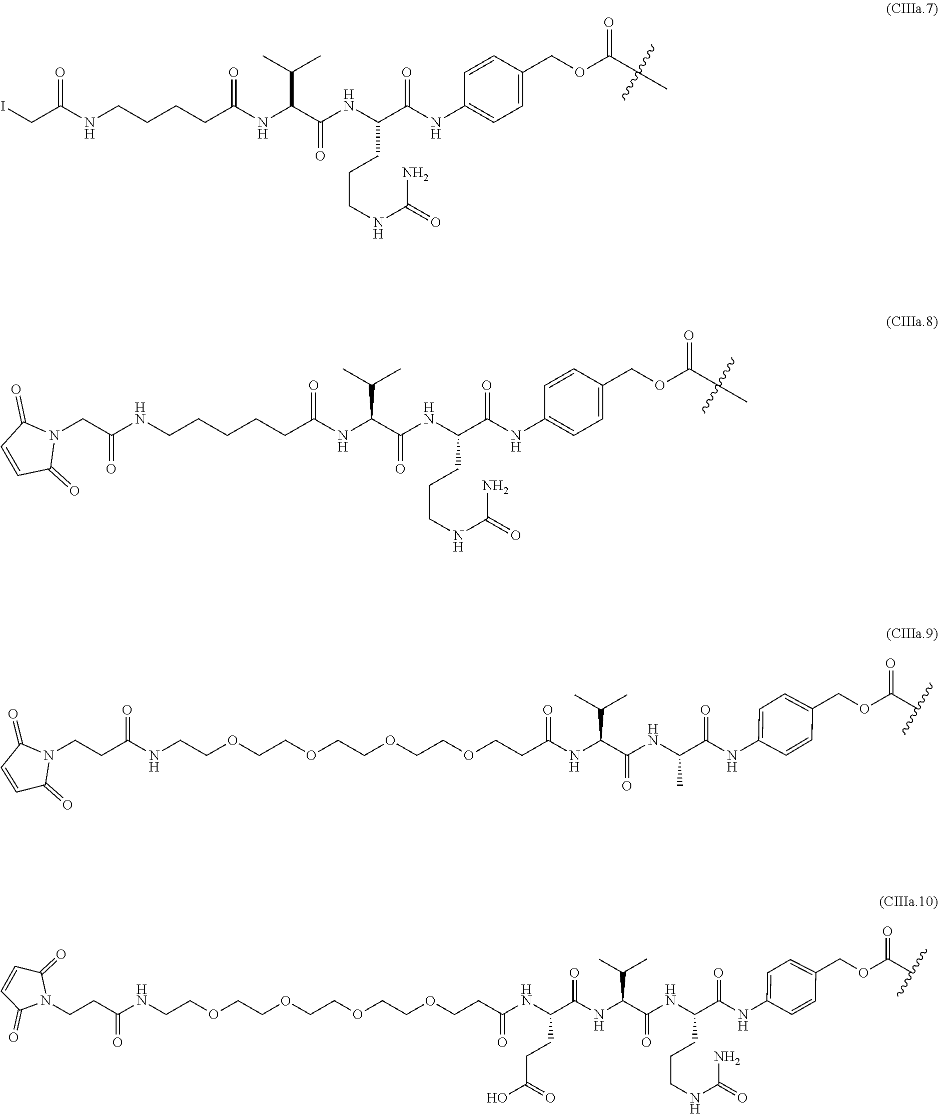 [SMILES: *C(=O)OCc1ccc(NC(=O)[C@H](CCCNC(N)=O)NC(=O)[C@@H](NC(=O)CCCCNC(=O)CI)C(C)C)cc1 ; *C(=O)OCc1ccc(NC(=O)[C@H](CCCNC(N)=O)NC(=O)[C@@H](NC(=O)CCCCCNC(=O)CN2C(=O)C=CC2=O)C(C)C)cc1 ; *C(=O)OCc1ccc(NC(=O)[C@H](C)NC(=O)[C@@H](NC(=O)CCOCCOCCOCCOCCNC(=O)CCN2C(=O)C=CC2=O)C(C)C)cc1 ; *C(=O)OCc1ccc(NC(=O)[C@H](CCCNC(N)=O)NC(=O)[C@@H](NC(=O)[C@H](CCC(=O)O)NC(=O)CCOCCOCCOCCOCCNC(=O)CCN2C(=O)C=CC2=O)C(C)C)cc1]

wherein [symbol] indicates an attachment site of a linker (L) to a compound of Formula (I), (II), (III), (IV), (V), or (VI), or pharmaceutically acceptable isomer, tautomer, racemate, hydrate, solvate, isotope, or salt thereof.

Exemplary embodiments of linkers according to structural formula (CIIIb), (CIIIc), or (CIIId) that can be included in the conjugates can include the linkers illustrated below (as illustrated, the linkers include a reactive group suitable for covalently linking the linker to an antibody construct):

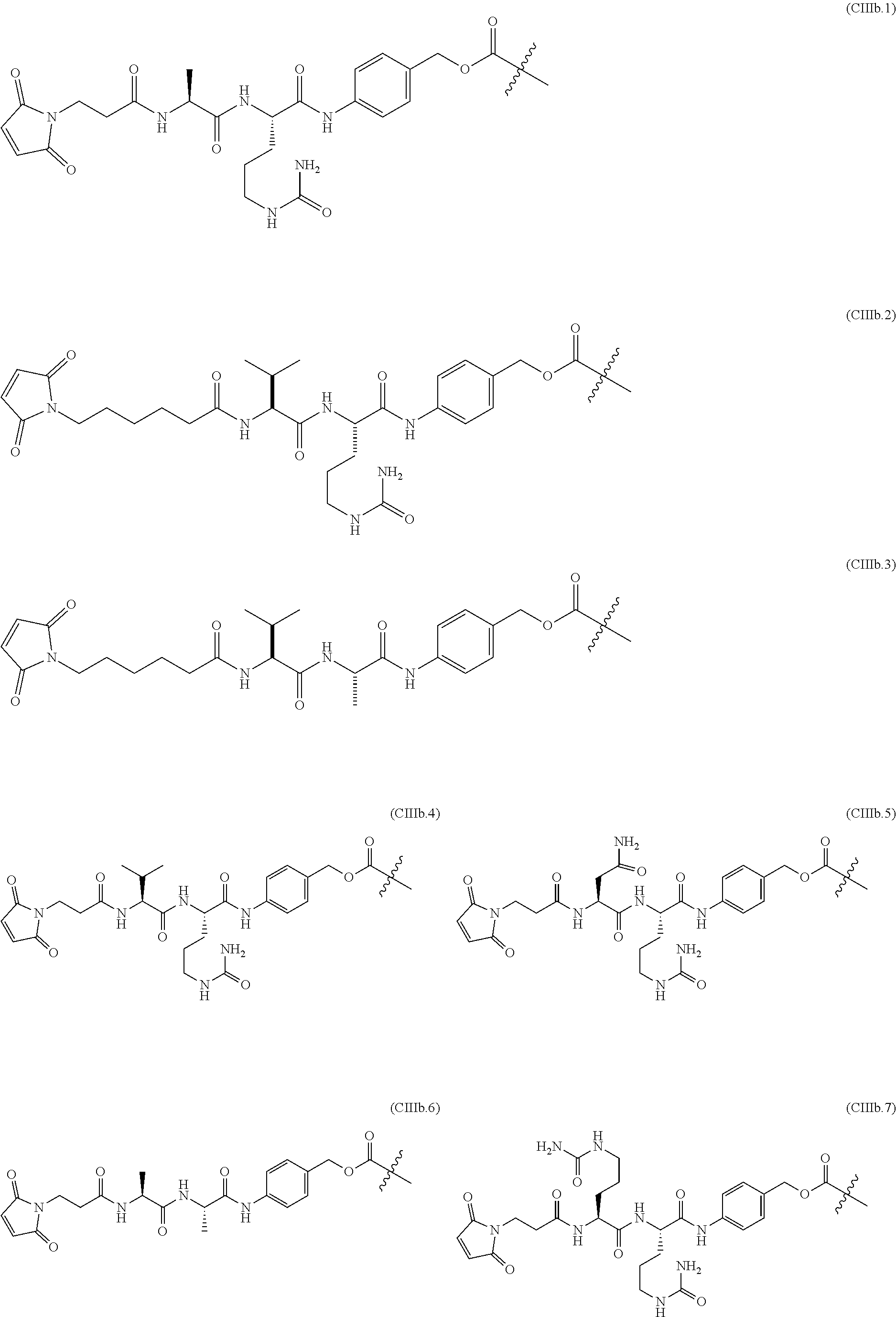

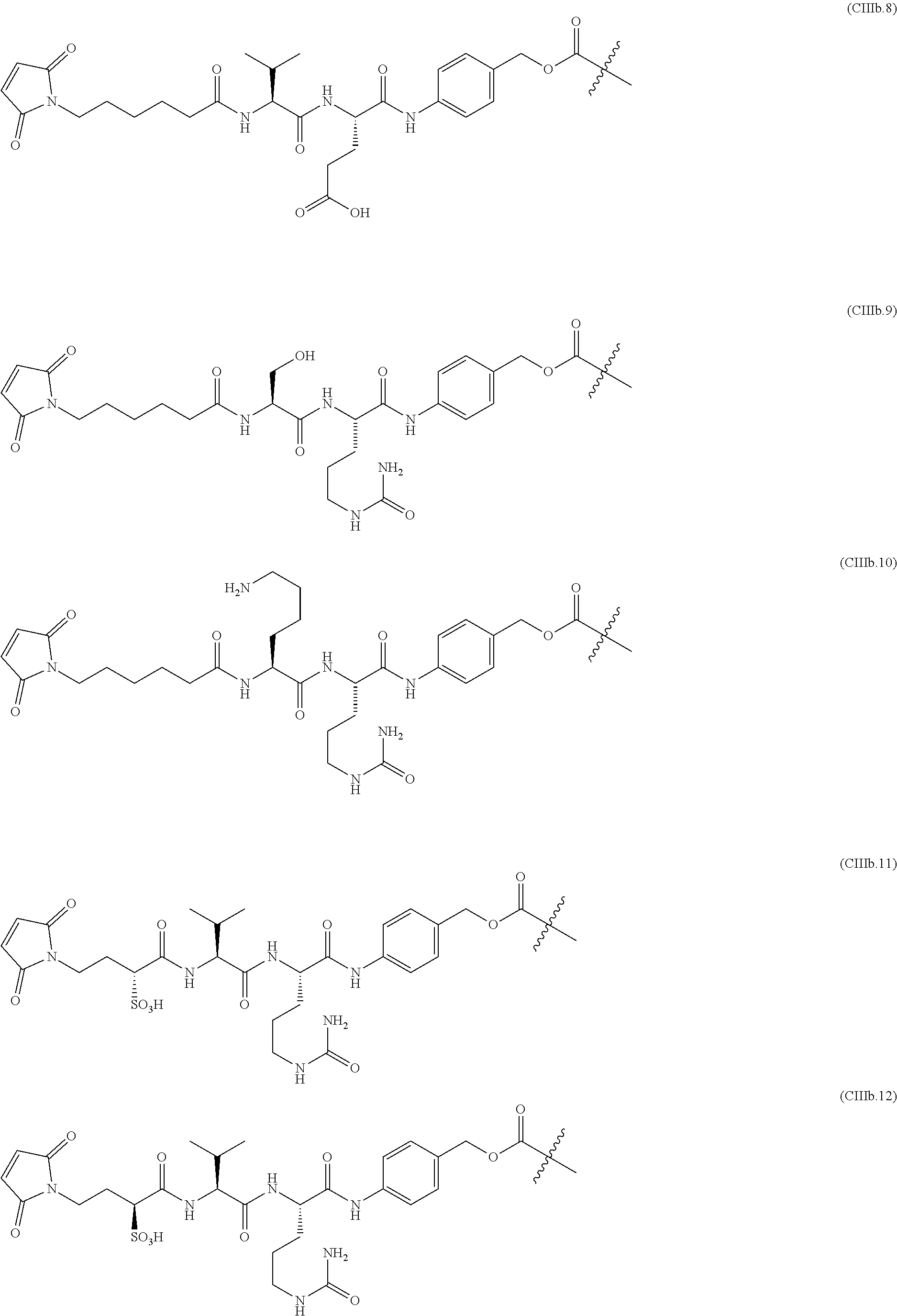 [SMILES: *C(=O)OCc1ccc(NC(=O)[C@H](CCC(=O)O)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@H](CO)NC(=O)CCCCCN2C(=O)C=CC2=O)cc1 ; *C(=O)OCc1ccc(NC(=O)[C@H](CCCNC(N)=O)NC(=O)[C@H](CCCCN)NC(=O)CCCCCN2C(=O)C=CC2=O)cc1 ; *C(=O)OCc1ccc(NC(=O)[C@H](CCCNC(N)=O)NC(=O)[C@@H](NC(=O)[C@@H](CCN2C(=O)C=CC2=O)S(=O)(=O)O)C(C)C)cc1 ; *C(=O)OCc1ccc(NC(=O)[C@H](CCCNC(N)=O)NC(=O)[C@@H](NC(=O)[C@H](CCN2C(=O)C=CC2=O)S(=O)(=O)O)C(C)C)cc1]

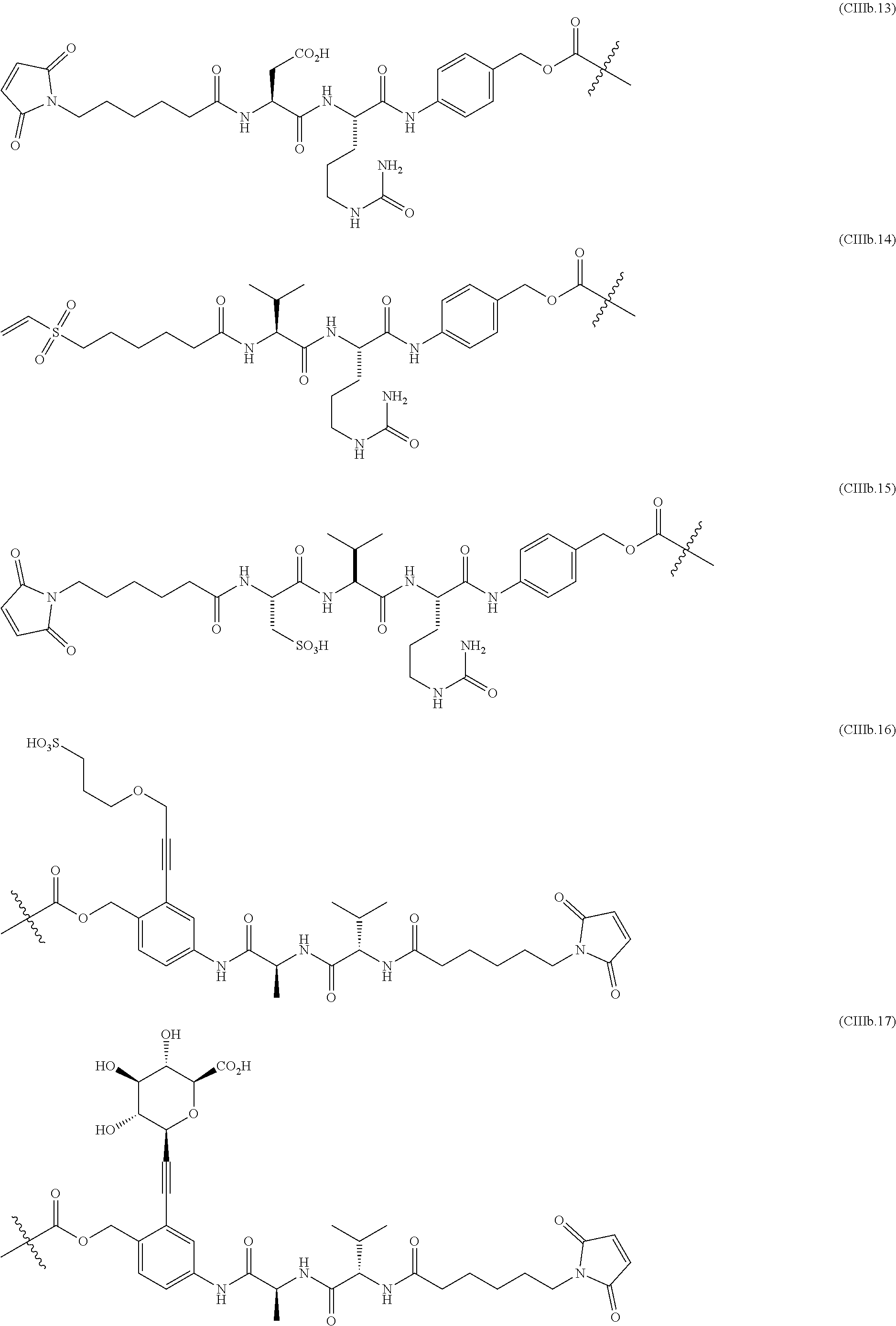

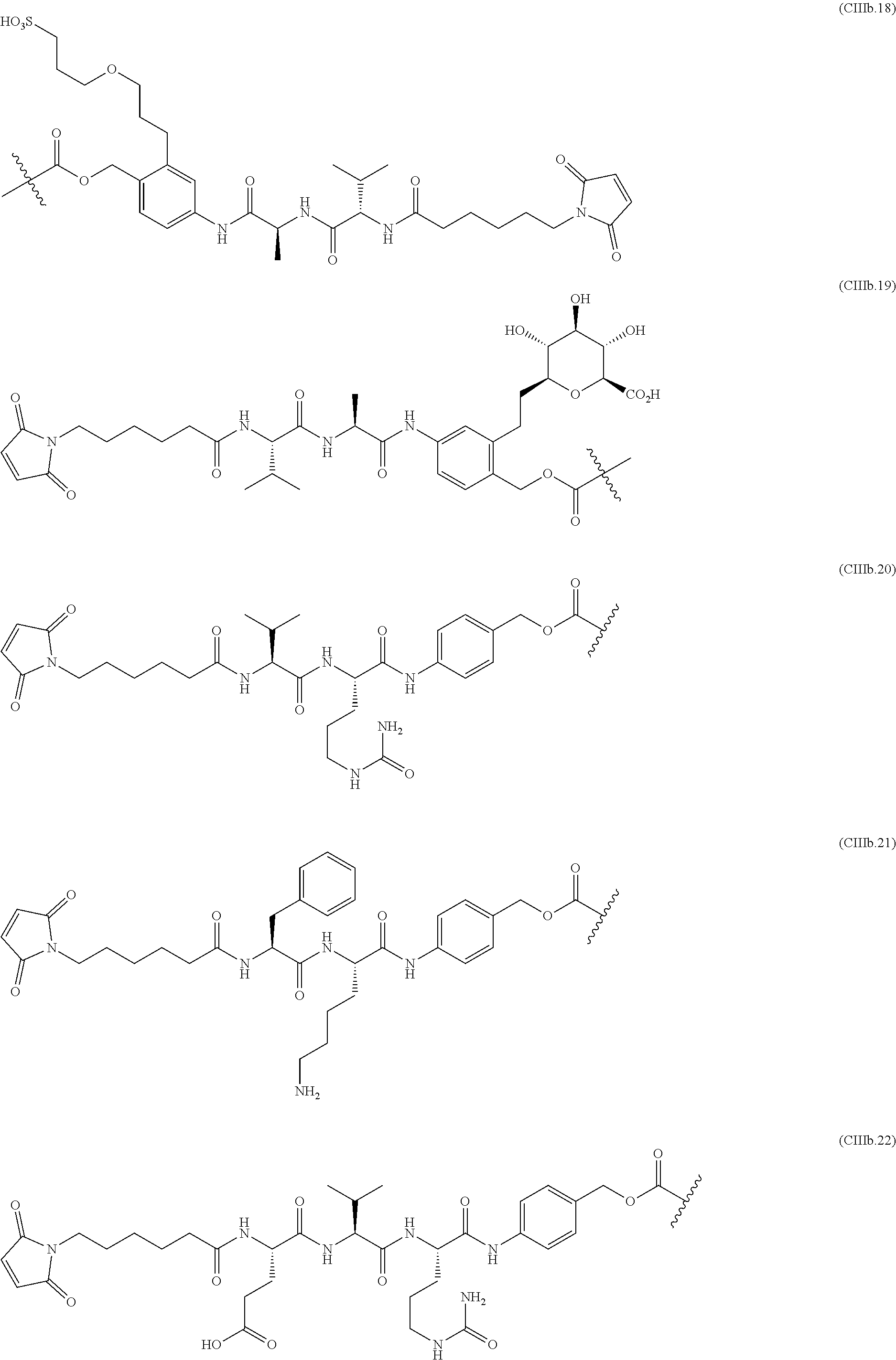

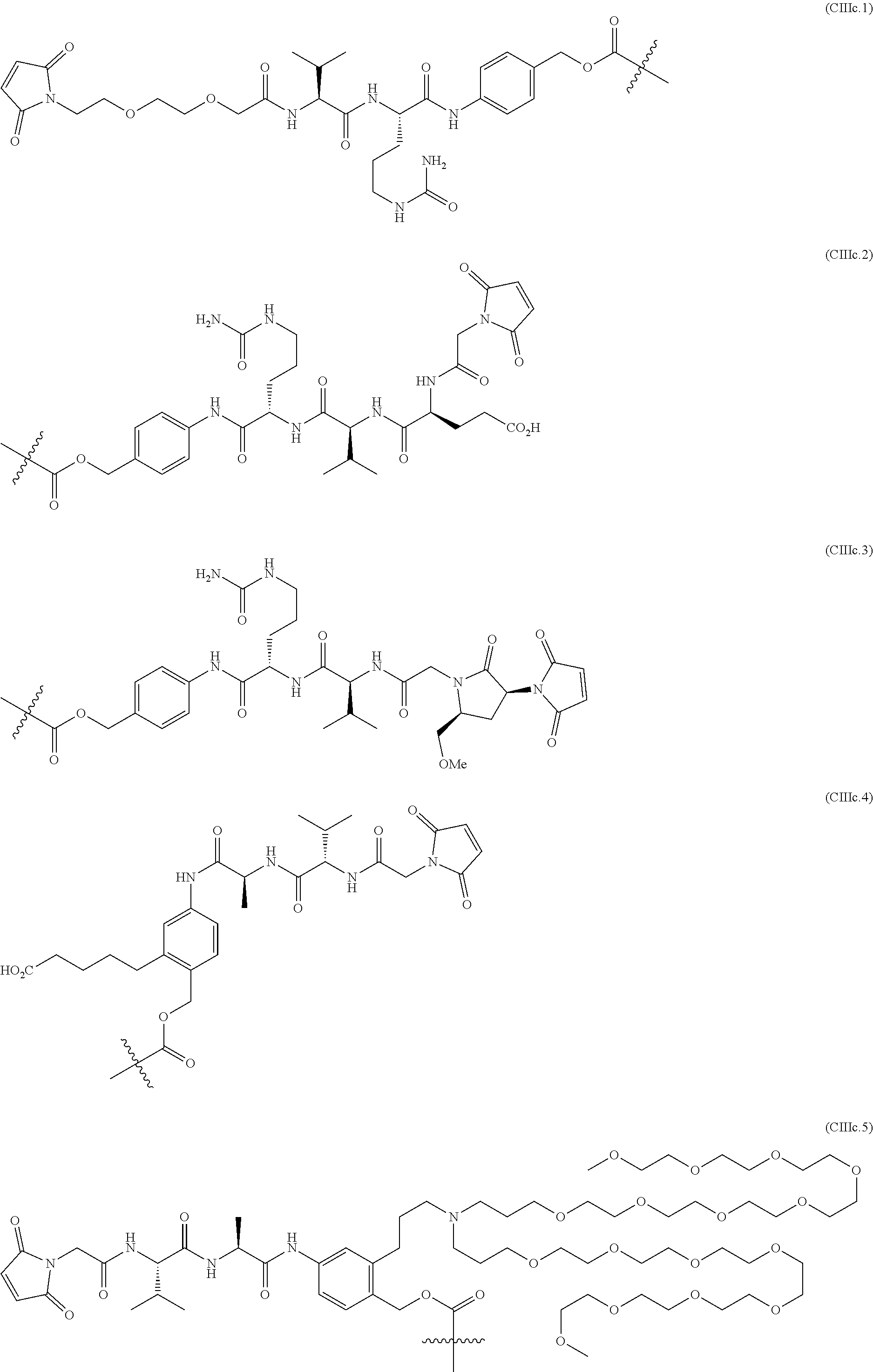

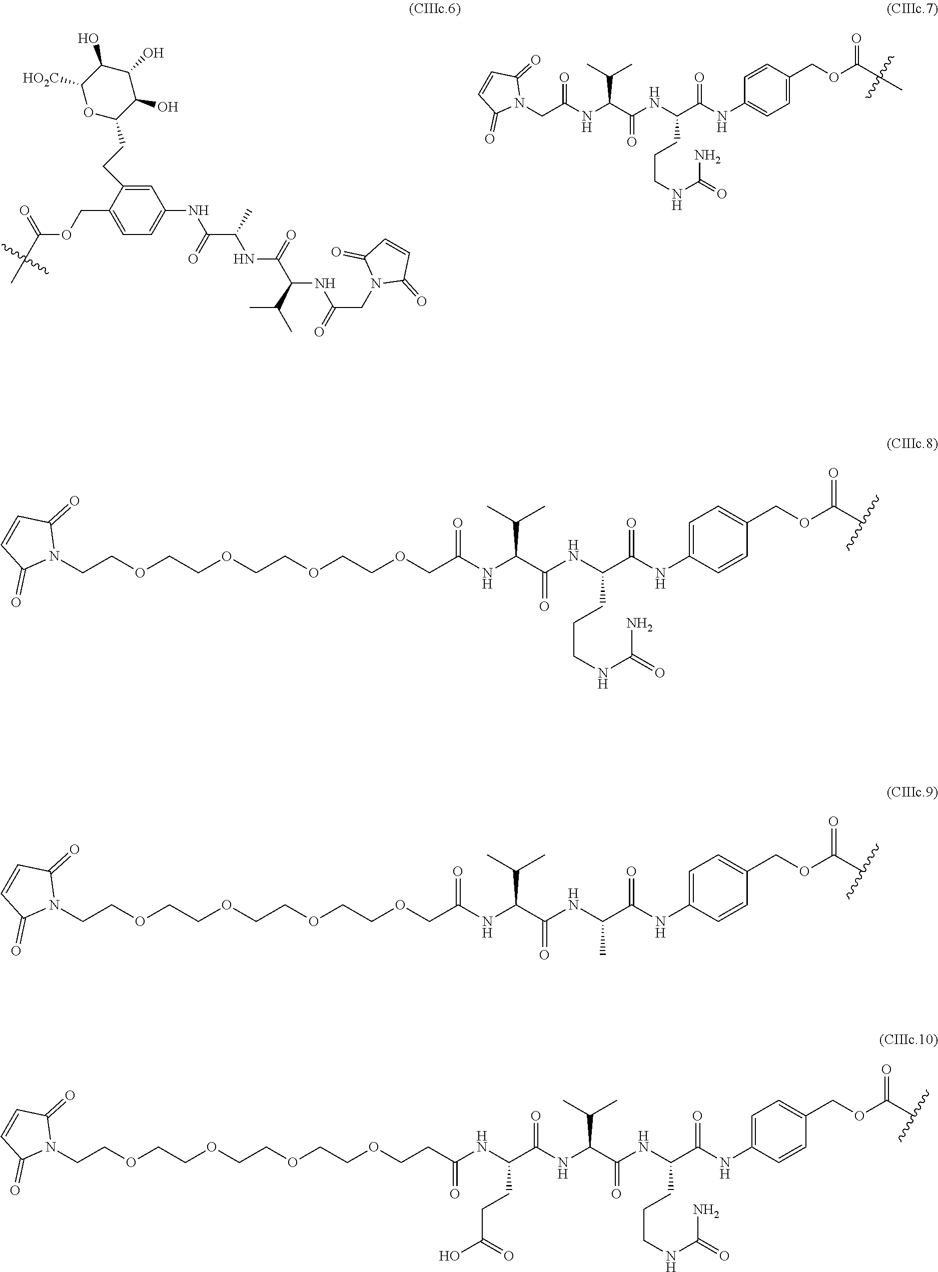

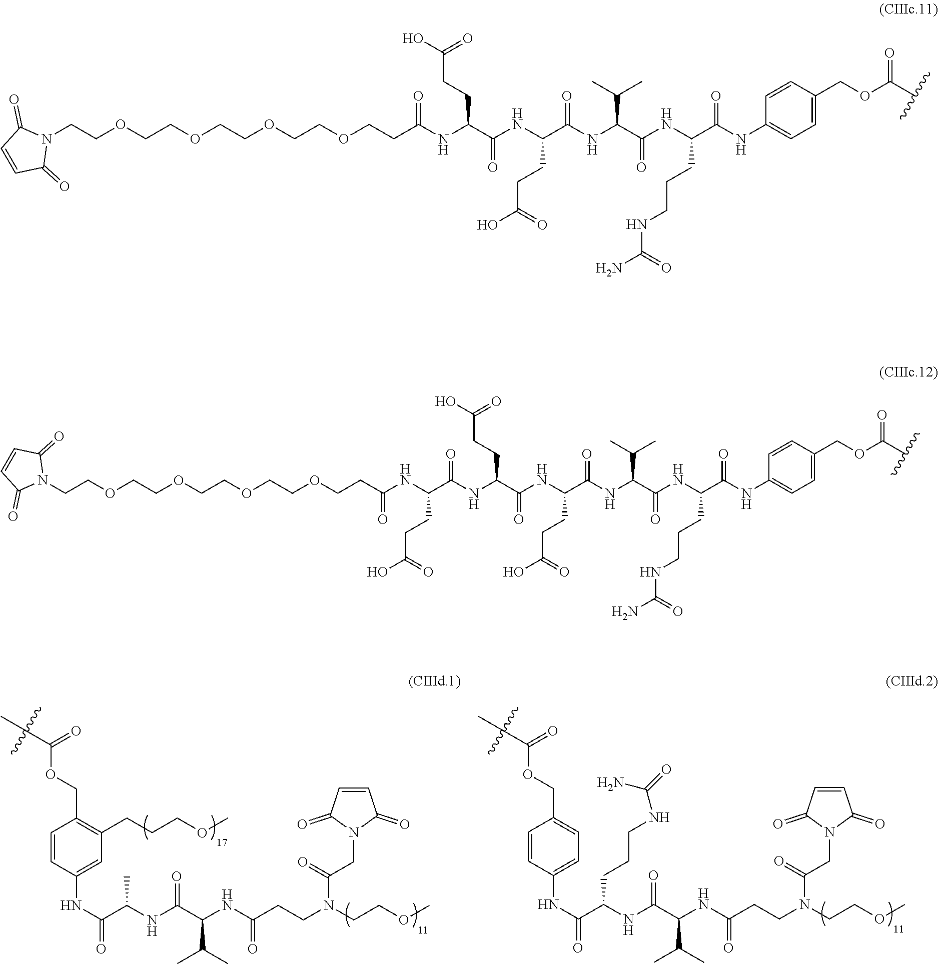

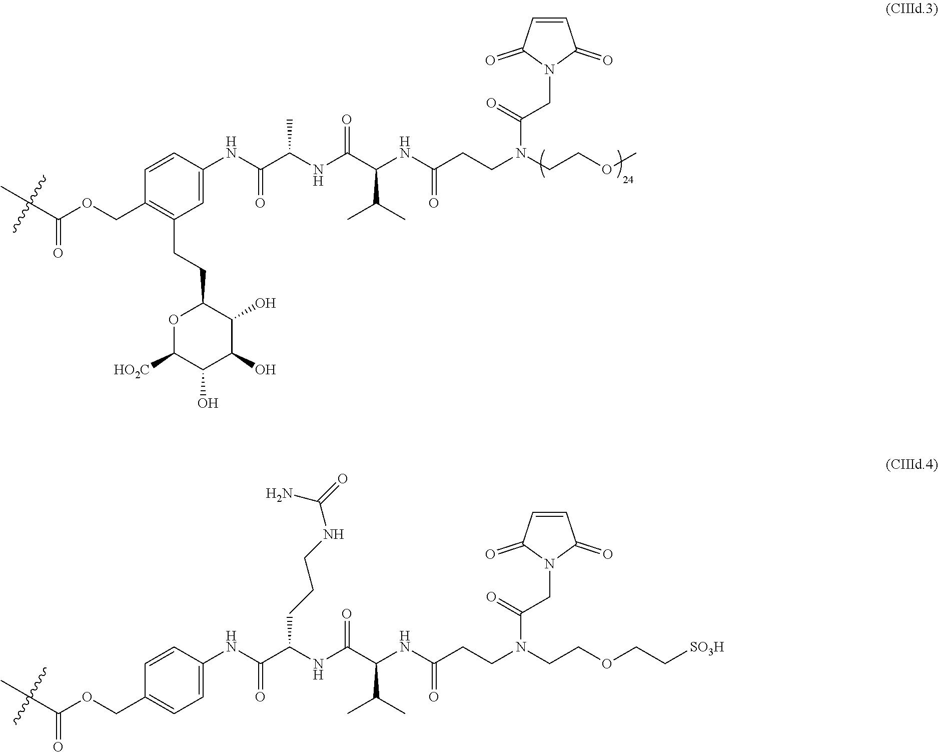

wherein [symbol] indicates an attachment site to a TGFβR1 inhibitor, or pharmaceutically acceptable isomer, tautomer, racemate, hydrate, solvate, isotope, or salt thereof.

The enzymatically cleavable linker can be a ß-glucuronic acid-based linker. Facile release of the TGFβR1 inhibitor can be realized through cleavage of the ß-glucuronide glycosidic bond by the lysosomal enzyme ß-glucuronidase. This enzyme can be abundantly present within lysosomes and can be overexpressed in some tumor types, while the enzyme activity outside cells can be low. ß-Glucuronic acid-based linkers can be used to circumvent the tendency of a conjugate to undergo aggregation due to the hydrophilic nature of ß-glucuronides. In certain embodiments, ß-glucuronic acid-based linkers can link the antibody construct to a hydrophobic TGFβR1 inhibitor.

A variety of cleavable β-glucuronic acid-based linkers useful for linking drugs such as auristatins, camptothecin and doxorubicin analogues, CBI minor-groove binders, and psymberin to antibodies have been described. All of these β-glucuronic acid-based linkers may be used in the conjugates comprising a TGFβR1 inhibitor described herein. In certain embodiments, the enzymatically cleavable linker is a β-galactoside-based linker. β-Galactoside is present abundantly within lysosomes, while the enzyme activity outside cells is low.

Additionally, cleavable linkers may comprise a phenol and connection through the phenolic oxygen. One such linker employs diamino-ethane unit (e.g., “Space Link”) in conjunction with traditional “PABO”-based self-immolative groups to deliver a phenol. Inhibitors containing an aromatic or aliphatic hydroxyl group can be covalently bonded to a linker through the hydroxyl group using a methodology that relies on a methylene carbamate linkage, as described in WO 2015/095755.

Other degradable linkages that can be included in cleavable linkers include ester linkages formed by the reaction of PEG carboxylic acids or activated PEG carboxylic acids with alcohol groups on a TGFβR1 inhibitor, wherein such ester groups can hydrolyze under physiological conditions to release the TGFβR1 inhibitor. Hydrolytically degradable linkages can include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LPXTG (SEQ ID NO:237) recognition motif to an N-terminal GGG motif to regenerate a native amide bond. The linker created can therefore link a moiety attached to the LPXTG (SEQ ID NO:237) recognition motif with a moiety attached to the N-terminal GGG motif.

A linker can be a link created between an unnatural amino acid on one moiety reacting with oxime bond that was formed by modifying a ketone group with an alkoxyamine on another moiety. A moiety can be an antibody construct. A moiety can be a binding domain. A moiety can be an antibody. A moiety can be a TGFβR1 inhibitor.

A linker can be attached to an antibody construct at any suitable site, such as for example at a terminus of an amino acid sequence or at a side chain of a cysteine residue, an engineered cysteine residue, a lysine residue, a serine residue, a threonine residue, a tyrosine residue, an aspartic acid residue, a glutamic acid residue, a glutamine residue, an engineered glutamine residue, a selenocysteine residue, or a non-natural amino acid. Non-natural amino acids can include para-azidomethyl-1-phenylalanine (pAMF). An attachment site can also be at a residue containing an oxime bond that was formed by modifying a ketone group with an alkoxyamine on another moiety, and a reactive primary amine, such as a reactive primary amine at a C-terminal end of a protein or peptide, such as by using Sortase A linker, which can be created by a Sortase A enzyme fusing an LXPTG recognition motif (SEQ ID NO:237) to an N-terminal GGG motif to regenerate a native amide bond. The linker created can therefore link a moiety attached to the LXPTG recognition motif (SEQ ID NO:237) with a moiety attached to the N-terminal GGG motif.

A direct linkage can be a covalent bond. For example, a linker can be attached to a terminus of an amino acid sequence of an antibody construct, or could be attached to a side chain modification to the antibody construct, such as example at a side chain of a cysteine residue, an engineered cysteine residue, a lysine residue, a serine residue, a threonine residue, a tyrosine residue, an aspartic acid residue a glutamic acid residue, a glutamine residue, an engineered glutamine residue, a selenocysteine residue, or a non-natural amino acid. Non-natural amino acids can include para-azidomethyl-1-phenylalanine (pAMF). An attachment can also be at a residue containing an oxime bond that was formed by modifying a ketone group with an alkoxyamine on another moiety, and a reactive primary amine, such as a reactive primary amine at a C-terminal end of a protein or peptide, such as by using Sortase A linker, which can be created by a Sortase A enzyme fusing an LXPTG recognition motif (SEQ ID NO:237) to an N-terminal GGG motif to regenerate a native amide bond. The linker created can therefore link a moiety attached to the LXPTG recognition motif (SEQ ID NO:237) with a moiety attached to the N-terminal GGG motif. An attachment can be via any of a number of bonds, for example but not limited to, an amide bond, an ester bond, an ether bond, a carbon-nitrogen bond, a carbon-carbon single double or triple bond, a disulfide bond, or a thioether bond. A linker can have at least one functional group, which can be linked to the antibody construct. Non-limiting examples of the functional groups can include those which form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups.

An attachment can be via any of a different types of bonds, for example but not limited to, an amide bond, an ester bond, an ether bond, a carbon-nitrogen bond, a carbon-carbon single, double or triple bond, a disulfide bond, or a thioether bond. A linker can have at least one functional group, which can be linked to the antibody construct (e.g., an antibody). Non-limiting examples of the functional groups can include those which form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups. Attachment groups that are used to attach the linkers in a conjugate can be electrophilic in nature and include, for example, active esters such as NHS esters and HOBt esters, haloformates, acid halides, carboxylic acids, amines, alkyl halides, benzyl halides such as haloacetamides, maleimide groups, or activated disulfides.

A linker can be connected to an antibody construct at a hinge cysteine of an antibody Fc region or domain. A linker can be connected to an antibody construct at a light chain constant domain lysine. A linker can be connected to an antibody construct at an engineered cysteine in the light chain. A linker can be connected to an antibody construct at an engineered light chain glutamine. A linker can be connected to an antibody construct at an unnatural amino acid engineered into the light chain. A linker can be connected to an antibody construct at a heavy chain constant domain lysine. A linker can be connected to an antibody construct at an engineered cysteine in the heavy chain. A linker can be connected to an antibody construct at an engineered heavy chain glutamine. A linker can be connected to an antibody construct an unnatural amino acid engineered into the heavy chain. Amino acids can be engineered into an amino acid sequence of an antibody construct as described herein, for example, and can be connected to a linker of a conjugate. Engineered amino acids can be added to a sequence of existing amino acids. Engineered amino acids can be substituted for one or more existing amino acids of a sequence of amino acids.

A linker can be conjugated to an antibody construct via a sulfhydryl group. A linker can be conjugated to an antibody construct via a primary amine. A linker can be a link created between an unnatural amino acid on an antibody construct reacting with oxime bond that was formed by modifying a ketone group with an alkoxyamine on a TGFβR1 inhibitor. When a linker is connected to an antibody construct at the sites described herein, an Fc domain of the conjugate can bind to Fc receptors. When a linker is connected to an antibody construct at the sites described herein, the antigen binding domain of the conjugate can bind its antigen. When a linker is connected to an antibody construct at the sites described herein, a binding domain of the conjugate can bind its antigen.

An antibody with engineered reactive cysteine residues can be used to link a binding domain to the antibody. A linker can connect an antibody construct to a binding domain via Sortase A linker. A Sortase A linker can be created by a Sortase A enzyme fusing an LXPTG recognition motif (SEQ ID NO:237) to an N-terminal GGG motif to regenerate a native amide bond. The linker created can therefore link an antibody construct attached to the LXPTG recognition motif (SEQ ID NO:237) with a binding domain attached to the N-terminal GGG motif. A binding domain can be connected to a linker by a direct linkage. A direct linkage is a covalent bond. For example, a linker can be attached to a terminus of an amino acid sequence of a binding domain, or could be attached to a side chain modification to the binding domain, such as the side chain of a cysteine residue, an engineered cysteine residue, a lysine residue, a serine residue, a threonine residue, a tyrosine residue, an aspartic acid residue, a glutamic acid residue, a glutamine residue, an engineered glutamine residue, a selenocysteine residue, or a non-natural amino acid. Non-natural amino acids can include para-azidomethyl-1-phenylalanine (pAMF). An attachment can also be at a residue containing an oxime bond that was formed by modifying a ketone group with an alkoxyamine on another moiety, and a reactive primary amine, such as a reactive primary amine at a C-terminal end of a protein or peptide. An attachment can be via any of a number of bonds, for example but not limited to, an amide bond, an ester bond, an ether bond, a carbon-nitrogen bond, a carbon-carbon single double or triple bond, a disulfide bond, or a thioether bond. A linker can have at least one functional group, which can be linked to the binding domain. Non-limiting examples of the functional groups can include those which form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups. Amino acids can be engineered into an amino acid sequence of the binding domain. Engineered amino acids can be added to a sequence of existing amino acids. Engineered amino acids can be substituted for one or more existing amino acids of a sequence of amino acids. A linker can be conjugated to a binding domain via a sulfhydryl group. A linker can be conjugated to a binding domain via a primary amine. A binding domain can be conjugated to the C-terminal of an Fc domain of a conjugate.

An antibody or antibody construct with engineered reactive cysteine residues can be used to link a TGFβR1 inhibitor to the antibody construct. A linker can connect an antibody construct to a TGFβR1 inhibitor via linker. A linker can connect an antibody construct to a TGFβR1 inhibitor via Sortase A linker. A Sortase A linker can be created by a Sortase A enzyme fusing an LXPTG recognition motif (SEQ ID NO:237) to an N-terminal GGG motif to regenerate a native amide bond. The linker created can therefore link an antibody attached the LXPTG recognition motif (SEQ ID NO:237) with a TGFβR1 inhibitor attached to the N-terminal GGG motif. A linker can be a link created between an unnatural amino acid an antibody reacting with oxime bond that was formed by modifying a ketone group with an alkoxyamine on a TGFβR1 inhibitor. The TGFβR1 inhibitor can comprise one or more rings selected from carbocyclic and heterocyclic rings. The TGFβR1 inhibitor can be covalently bound to a linker by a bond to an exocyclic carbon or nitrogen atom on the TGFβR1 inhibitor. A linker can be conjugated to a TGFβR1 inhibitor via an exocyclic nitrogen or carbon atom of a TGFβR1 inhibitor.

A linker-inhibitor conjugate can dissociate under physiological conditions to yield an active inhibitor. A linker can be connected to a TGFβR1 inhibitor by a direct linkage between the TGFβR1 inhibitor and the linker.

In some embodiments, a TGFβR1 inhibitor-linker can be formed by conjugating a noncleavable maleimide-PEG4 linker containing a succinimide group with a TGFβR1 inhibitor-linker.

A non-cleavable linker can be protease insensitive. A non-cleavable linker can contain a succinimide group. A non-cleavable spacer can comprise succinimidocaproyl group. A succinimidocaproyl linker can comprise N-succinimidomethylcyclohexane-1-carboxylate.

A non-cleavable linker can be a combination of a succinimidocaproyl group and one or more ethylene glycol units. A non-cleavable linker can be a succinimide-PEG4 linker. A non-cleavable linker can be a combination of a succinimidocaproyl linker containing a succinimide group and one or more ethylene glycol units. A non-cleavable linker can be a combination of a succinimidocaproyl group, and one or more ethylene glycol units. A non-cleavable linker can contain one or more succinimido groups linked to polyethylene glycol units in which the polyethylene glycol can allow for more linker flexibility or can be used lengthen the linker.

A linker can be a (succinimidocaproyl)-(valine-citrulline)-(para-aminobenzyloxycarbonyl) linker. A linker can be a linker suitable for attachment to an engineered cysteine (THIOMAB), such as a (succinimidocaproyl)-(valine-citrulline)-(para-aminobenzyloxycarbonyl)-linker.

In some embodiments, a non-cleavable linker independently comprises a heteroalkylene, heteroalkenylene, or heteroalkynylene comprising 3 to 30 components in the longest linear chain, wherein the components are selected from —CH₂—, —CH(C₁₋₄alkyl), —C(C₁₋₄alkyl)₂, —CH═CH—, —C≡C—, —C(O)—, —O—, —NH—, —N(C₁₋₄alkyl), —S—, —S(O)—, —S(O)₂—, and —P(O)(O⁻)—. In some embodiments, a non-cleavable linker independently comprises a heteroalkylene comprising 3 to 15 components in the longest linear chain, wherein the components are selected from —CH₂—, —CH(C₁₋₄alkyl), —C(O)—, —O—, —NH—, and —N(C₁₋₄alkyl). In some embodiments, the non-cleavable linker comprises a polyethylene glycol region comprising two to six ethylene glycol units. In some embodiments, a non-cleavable linker is independently —NH—C(O)—(CH₂)₂₋₅—C(O)—, —NH—C(O)—(CH₂)₂₋₅—C(O)NH—(CH₂)₂₋₇—C(O)—, or —NH—C(O)—(CH₂)₂₋₅—C(O)NH—(CH₂)₂₋₇—NH—C(O)—(CH₂)₂₋₄—C(O)—. In some embodiments, at least one non-cleavable linker is —NH—C(O)—(CH₂)₃—C(O)—.

In some embodiments, Region L³ comprises or is the following structure:

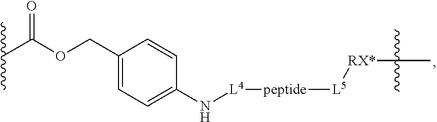

wherein:

L⁴ represents the C-terminus of the peptide;

L⁵ is selected from a bond, alkylene, and heteroalkylene, wherein L⁵ is optionally substituted with one or more groups independently selected from R³².

R³² is independently selected at each occurrence from halogen, —OH, —CN, —O— alkyl, —SH, ═O, ═S, —NH₂, —NO₂; and C₁₋₁₀ alkyl, C₂₋₁₀ alkenyl, C₂₋₁₀ alkynyl, each of which is optionally substituted with one or more substituents independently selected from halogen, —OH, —CN, —O-alkyl, —SH, ═O, ═S, —NH₂, —NO₂; and

RX* is a bond.

In some embodiments, the peptide comprises Val-Cit or Val-Ala.

A linker can comprise a cleavable peptide, for example, a linker comprising structural formula (IVa), (IVb), (IVc), or (IVd):

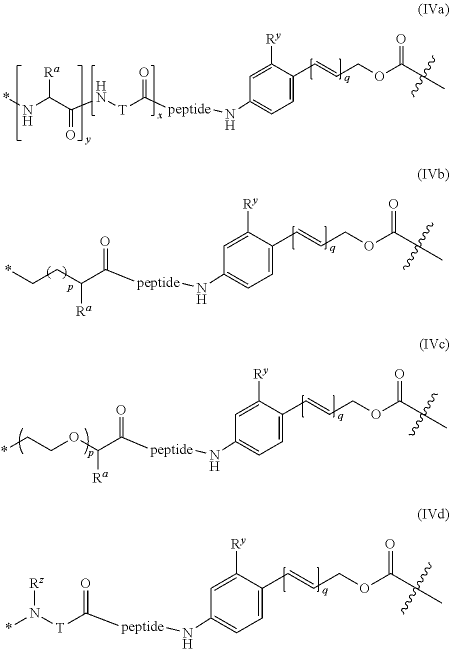

or a salt thereof, wherein:

peptide represents a cleavable peptide (illustrated N→C, wherein peptide includes the amino and carboxy “termini”) as described herein;

T represents a polymer comprising one or more ethylene glycol units or an alkylene chain, or combinations thereof;

R^{a} is selected from hydrogen, alkyl, sulfonate and methyl sulfonate;

R^{y} is hydrogen or C₁₋₄ alkyl-(O)ᵣ—(C₁₋₄ alkylene)ₛ-G¹ or C₁₋₄ alkyl-(N)-[(C₁₋₄ alkylene)-G¹]₂;

R^{z} is C₁₋₄ alkyl-(O)ᵣ—(C₁₋₄ alkylene)ₛ-G²

G¹ is SO₃H, CO₂H, PEG 4-32, or sugar moiety;

G² is SO₃H, CO₂H, or PEG 4-32 moiety;

r is 0 or 1;

s is 0 or 1;

p is an integer ranging from 0 to 5;

q is 0 or 1;

x is 0 or 1;

y is 0 or 1;

[symbol] represents one point of attachment of the linker to the inhibitor; and

* represents the point of attachment to the antibody construct moiety.

Exemplary polyvalent linkers that may be used to link inhibitors include Fleximer® linker technology that has the potential to enable high loading of conjugates with good physicochemical properties. The Fleximer® linker technology is based on incorporating drug molecules into a solubilizing poly-acetal backbone via a sequence of ester bonds.

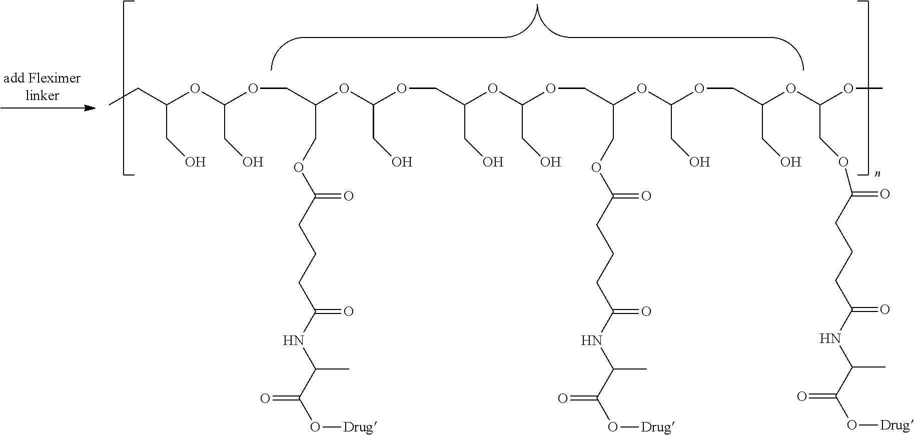

The methodology renders highly-loaded conjugates (drug loading up to 20) while maintaining good physicochemical properties. In certain embodiments, to utilize the Fleximer® linker technology depicted above, an aliphatic alcohol can be present or introduced into an inhibitor or salt thereof (e.g., an ALK5 inhibitor, as described herein). The alcohol moiety is then conjugated to an alanine moiety, which is then synthetically incorporated into the Fleximer® linker. Liposomal processing of the conjugate in vitro releases the parent alcohol-containing drug.

By way of example and not limitation, some connectors regions and connector segments comprising cleavable linkers and/or noncleavable linkers for the conjugates are described below.

Sulfamide linkers may also be used in the disclosed conjugates. Sulfamide linkers are as described herein and, e.g., U.S. Patent Publication No. 2019/0038765, the linkers of which are incorporated by reference herein.

Exemplary embodiments of linkers according to structural formula (IVa) that can be included in the conjugates described herein can include the structures illustrated below:

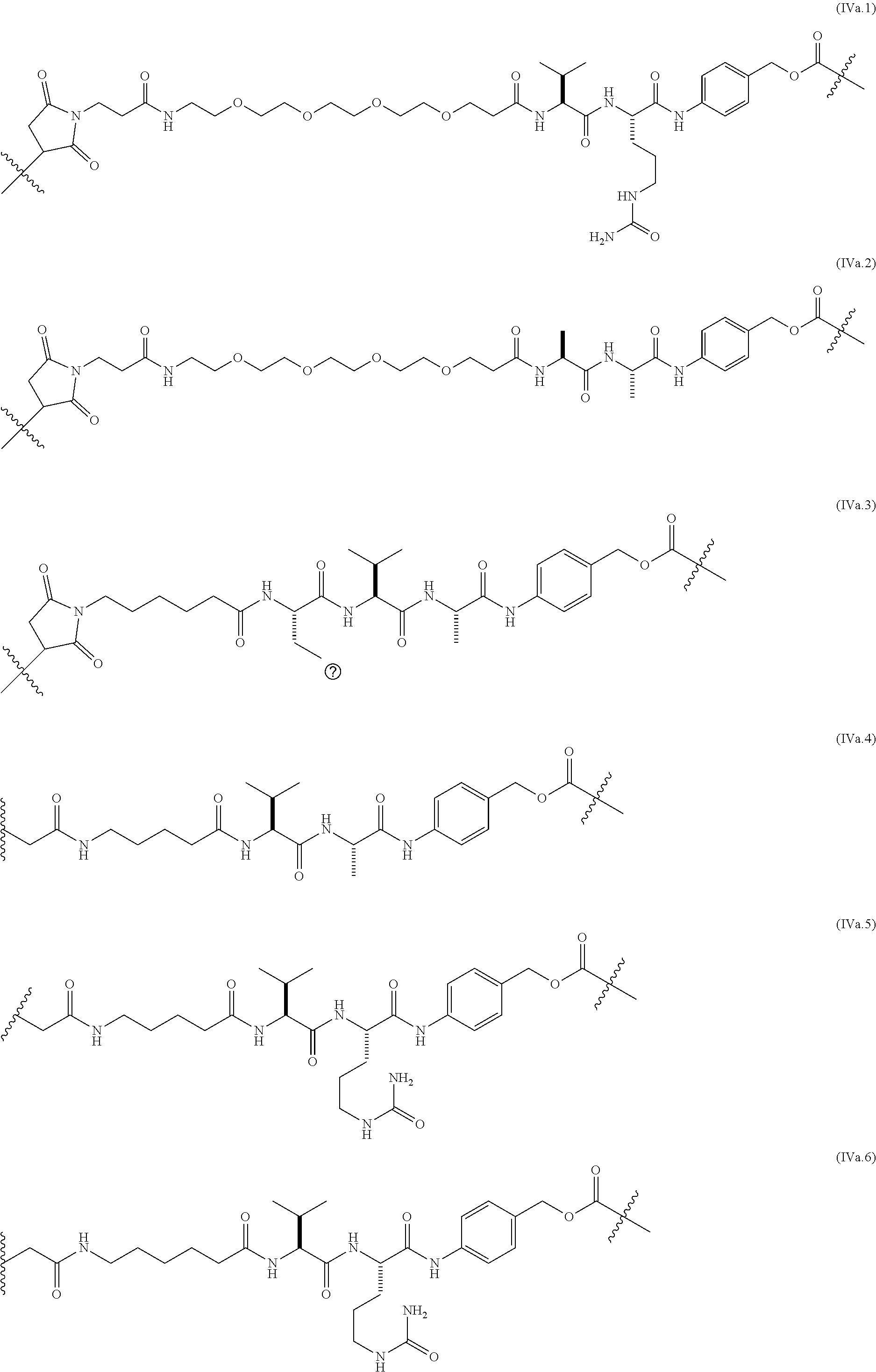

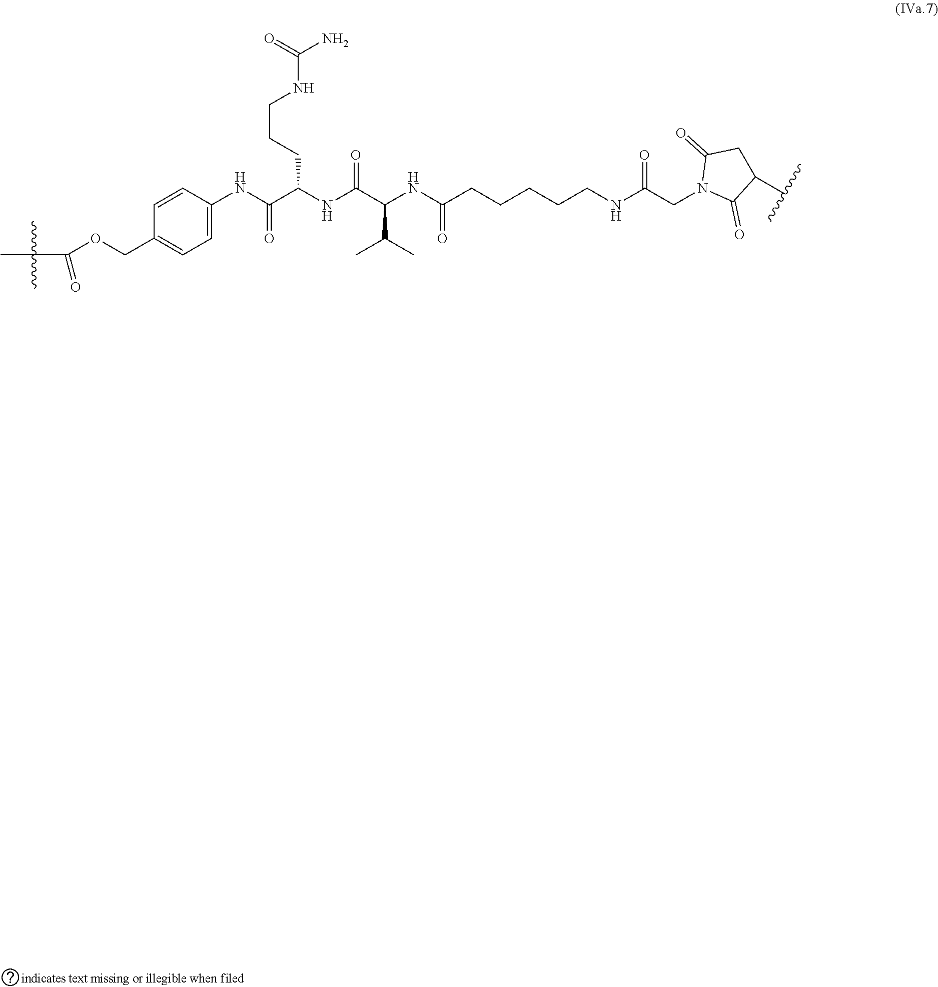 [SMILES: *C(=O)OCc1ccc(NC(=O)[C@H](CCCNC(N)=O)NC(=O)[C@@H](NC(=O)CCCCCNC(=O)CN2C(=O)CC(*)C2=O)C(C)C)cc1]

wherein one of [symbol] represents the point of attachment of the linker (L³) to the inhibitor.

Exemplary embodiments of connector regions or connector segments according to structural formula (IVb), (IVc), or (IVd) that can be included in the conjugates described herein can include the linkers illustrated below:

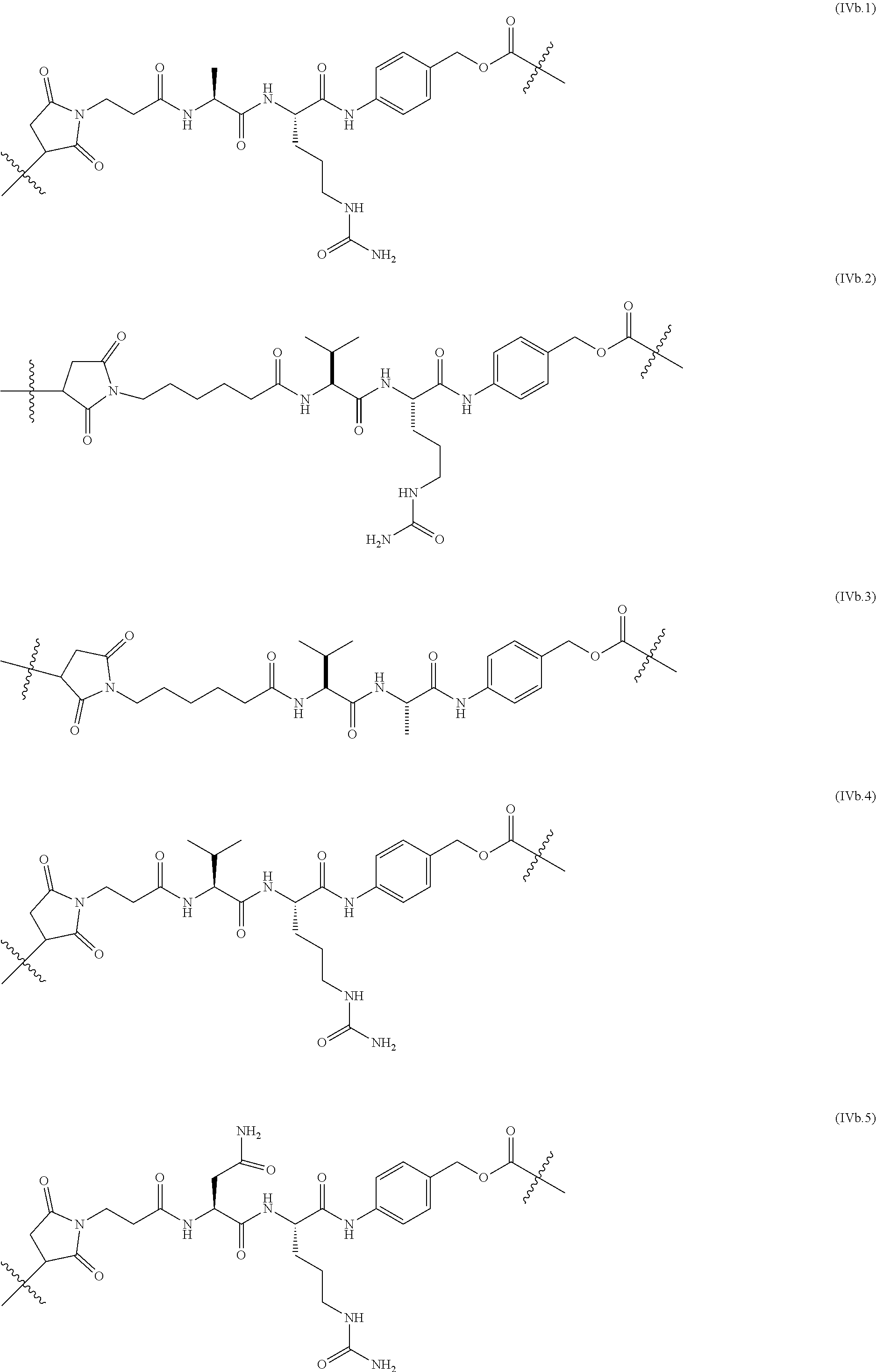 [SMILES: *C(=O)OCc1ccc(NC(=O)[C@H](CCCNC(N)=O)NC(=O)[C@H](C)NC(=O)CCN2C(=O)CC(*)C2=O)cc1 ; *C(=O)OCc1ccc(NC(=O)[C@H](CCCNC(N)=O)NC(=O)[C@@H](NC(=O)CCCCCN2C(=O)CC(*)C2=O)C(C)C)cc1 ; *C(=O)OCc1ccc(NC(=O)[C@H](C)NC(=O)[C@@H](NC(=O)CCCCCN2C(=O)CC(*)C2=O)C(C)C)cc1 ; *C(=O)OCc1ccc(NC(=O)[C@H](CCCNC(N)=O)NC(=O)[C@@H](NC(=O)CCN2C(=O)CC(*)C2=O)C(C)C)cc1 ; *C(=O)OCc1ccc(NC(=O)[C@H](CCCNC(N)=O)NC(=O)[C@H](CC(N)=O)NC(=O)CCN2C(=O)CC(*)C2=O)cc1]

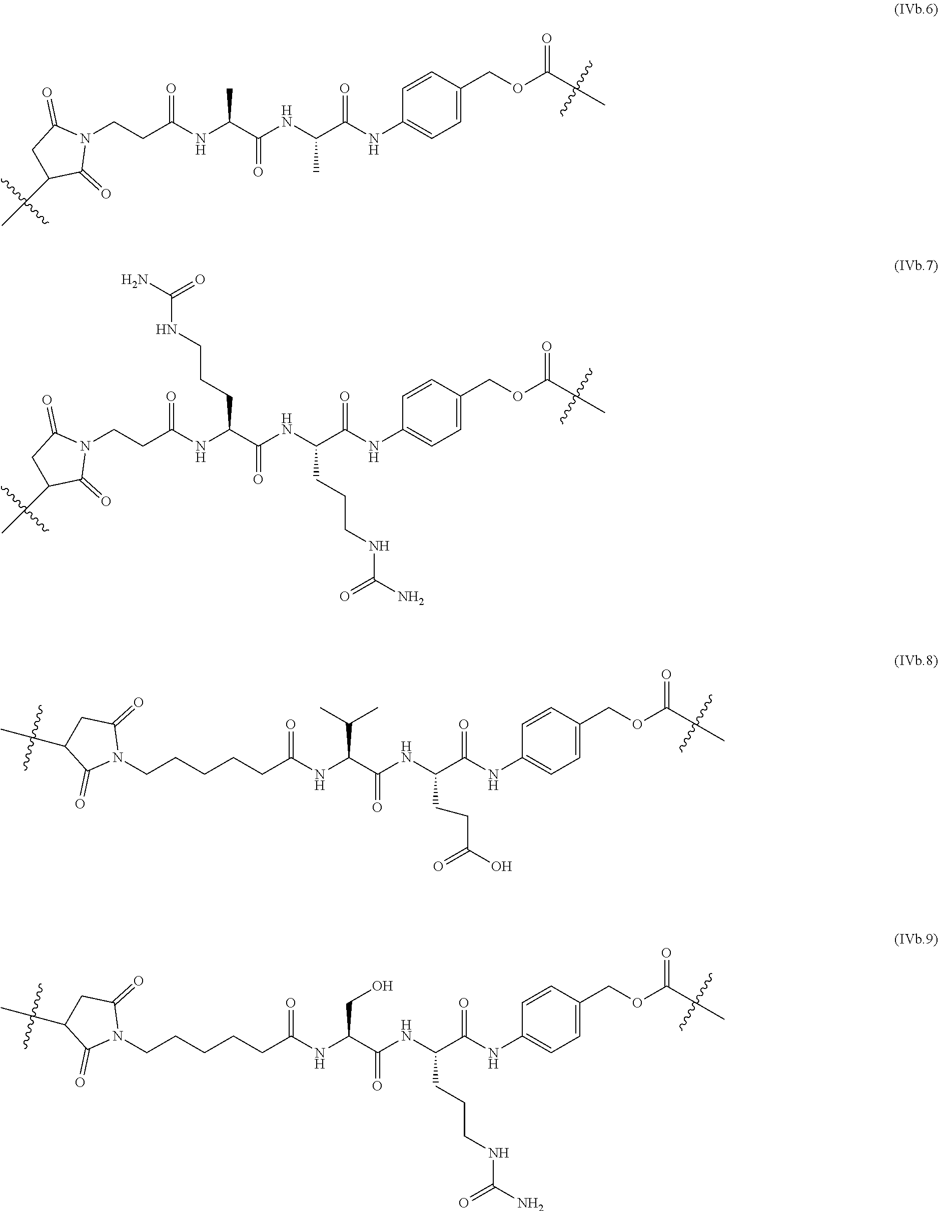 [SMILES: *C(=O)OCc1ccc(NC(=O)[C@H](C)NC(=O)[C@H](C)NC(=O)CCN2C(=O)CC(*)C2=O)cc1 ; *C(=O)OCc1ccc(NC(=O)[C@H](CCCNC(N)=O)NC(=O)[C@H](CCCNC(N)=O)NC(=O)CCN2C(=O)CC(*)C2=O)cc1 ; *C(=O)OCc1ccc(NC(=O)[C@H](CCC(=O)O)NC(=O)[C@@H](NC(=O)CCCCCN2C(=O)CC(*)C2=O)C(C)C)cc1 ; *C(=O)OCc1ccc(NC(=O)[C@H](CCCNC(N)=O)NC(=O)[C@H](CO)NC(=O)CCCCCN2C(=O)CC(*)C2=O)cc1]

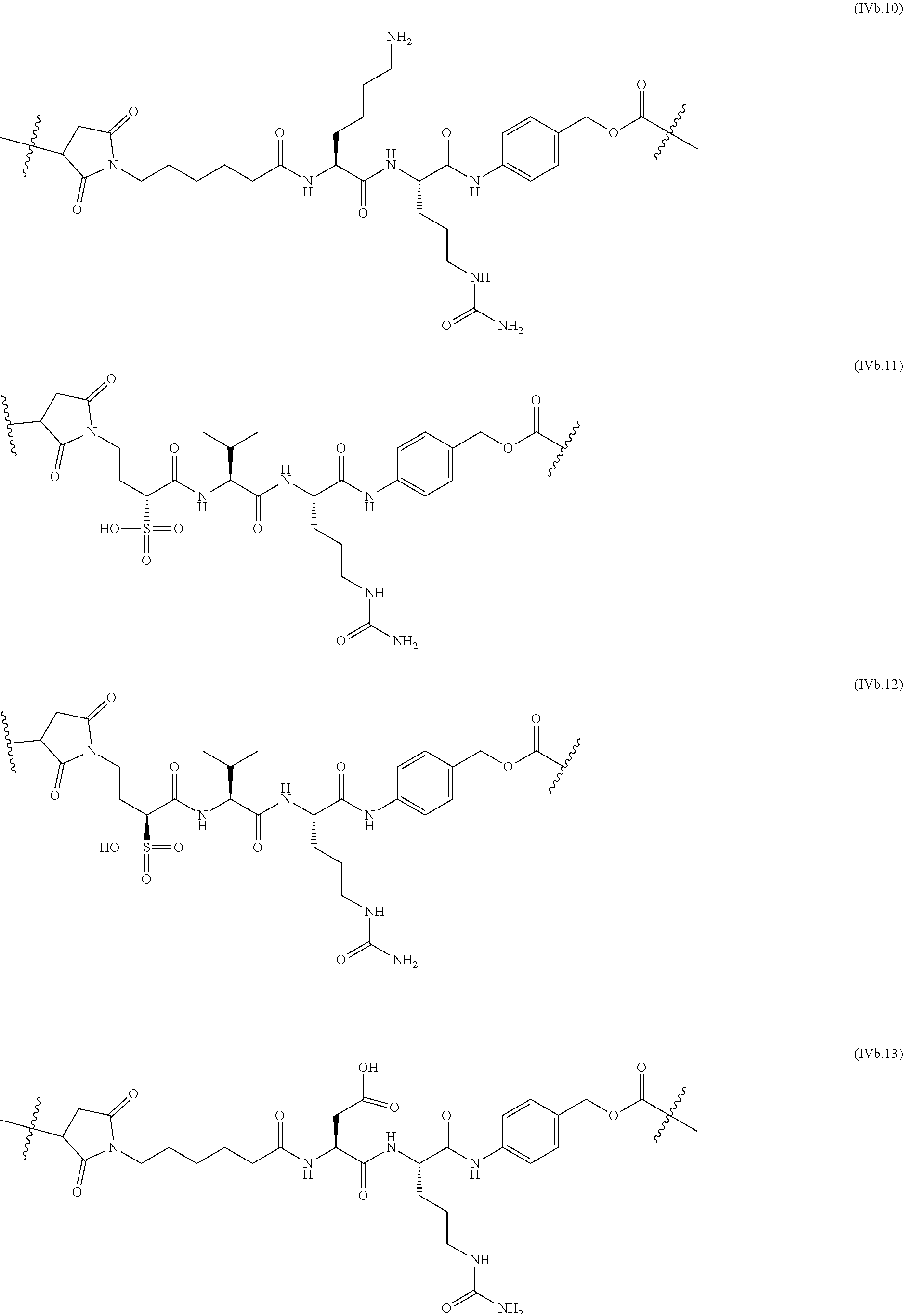 [SMILES: *C(=O)OCc1ccc(NC(=O)[C@H](CCCNC(N)=O)NC(=O)[C@H](CCCCN)NC(=O)CCCCCN2C(=O)CC(*)C2=O)cc1 ; *C(=O)OCc1ccc(NC(=O)[C@H](CCCNC(N)=O)NC(=O)[C@@H](NC(=O)[C@@H](CCN2C(=O)CC(*)C2=O)S(=O)(=O)O)C(C)C)cc1 ; *C(=O)OCc1ccc(NC(=O)[C@H](CCCNC(N)=O)NC(=O)[C@@H](NC(=O)[C@H](CCN2C(=O)CC(*)C2=O)S(=O)(=O)O)C(C)C)cc1 ; *C(=O)OCc1ccc(NC(=O)[C@H](CCCNC(N)=O)NC(=O)[C@H](CC(=O)O)NC(=O)CCCCCN2C(=O)CC(*)C2=O)cc1]

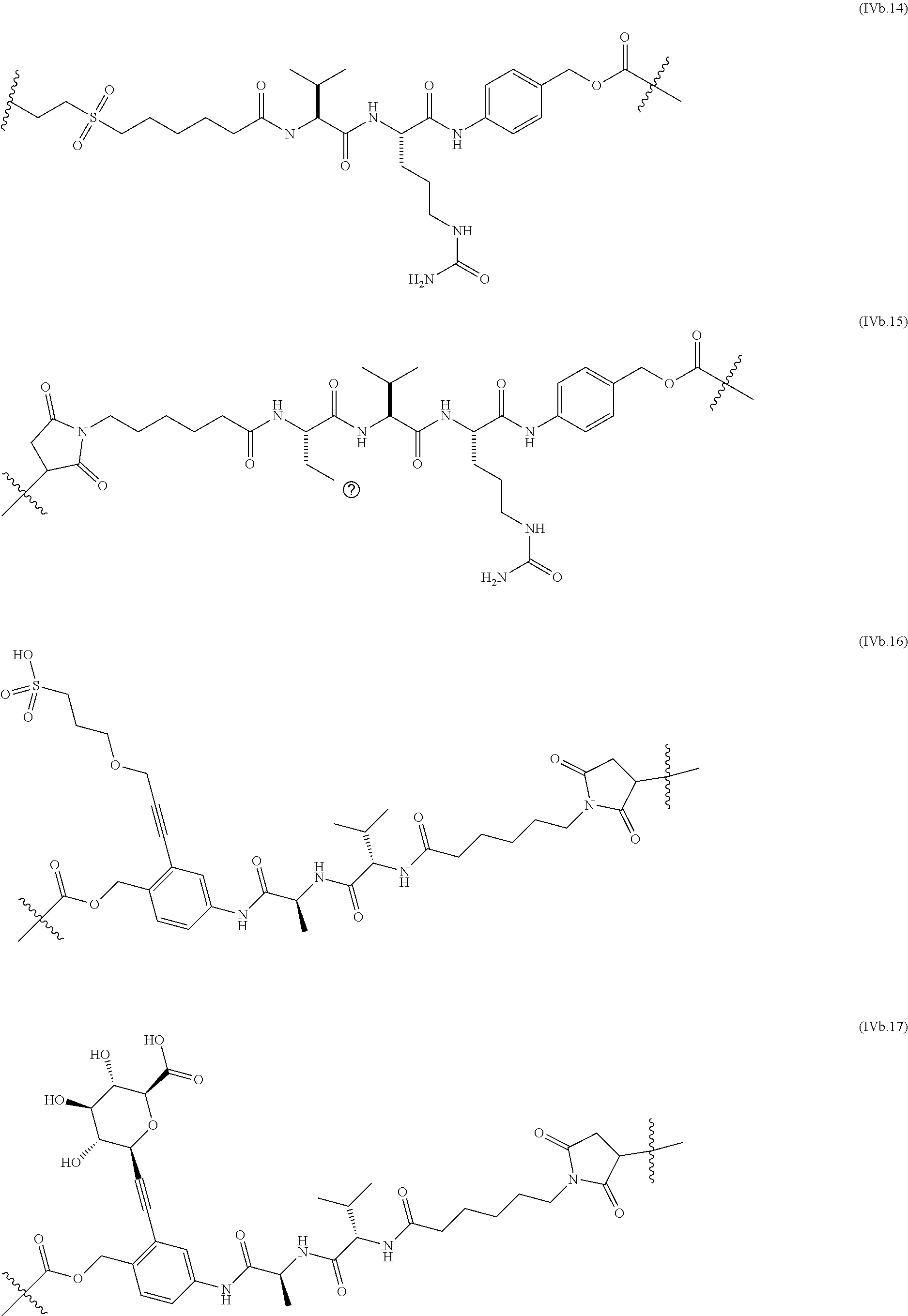 [SMILES: *CCS(=O)(=O)CCCCCC(=O)N[C@H](C(=O)N[C@@H](CCCNC(N)=O)C(=O)Nc1ccc(COC(*)=O)cc1)C(C)C ; *C(=O)OCc1ccc(NC(=O)[C@H](CCCNC(N)=O)NC(=O)[C@@H](NC(=O)[C@H](CC)NC(=O)CCCCCN2C(=O)CC(*)C2=O)C(C)C)cc1 ; *C(=O)OCc1ccc(NC(=O)[C@H](C)NC(=O)[C@@H](NC(=O)CCCCCN2C(=O)CC(*)C2=O)C(C)C)cc1C#CCOCCCS(=O)(=O)O ; *C(=O)OCc1ccc(NC(=O)[C@H](C)NC(=O)[C@@H](NC(=O)CCCCCN2C(=O)CC(*)C2=O)C(C)C)cc1C#C[C@@H]1O[C@H](C(=O)O)[C@@H](O)[C@H](O)[C@H]1O]

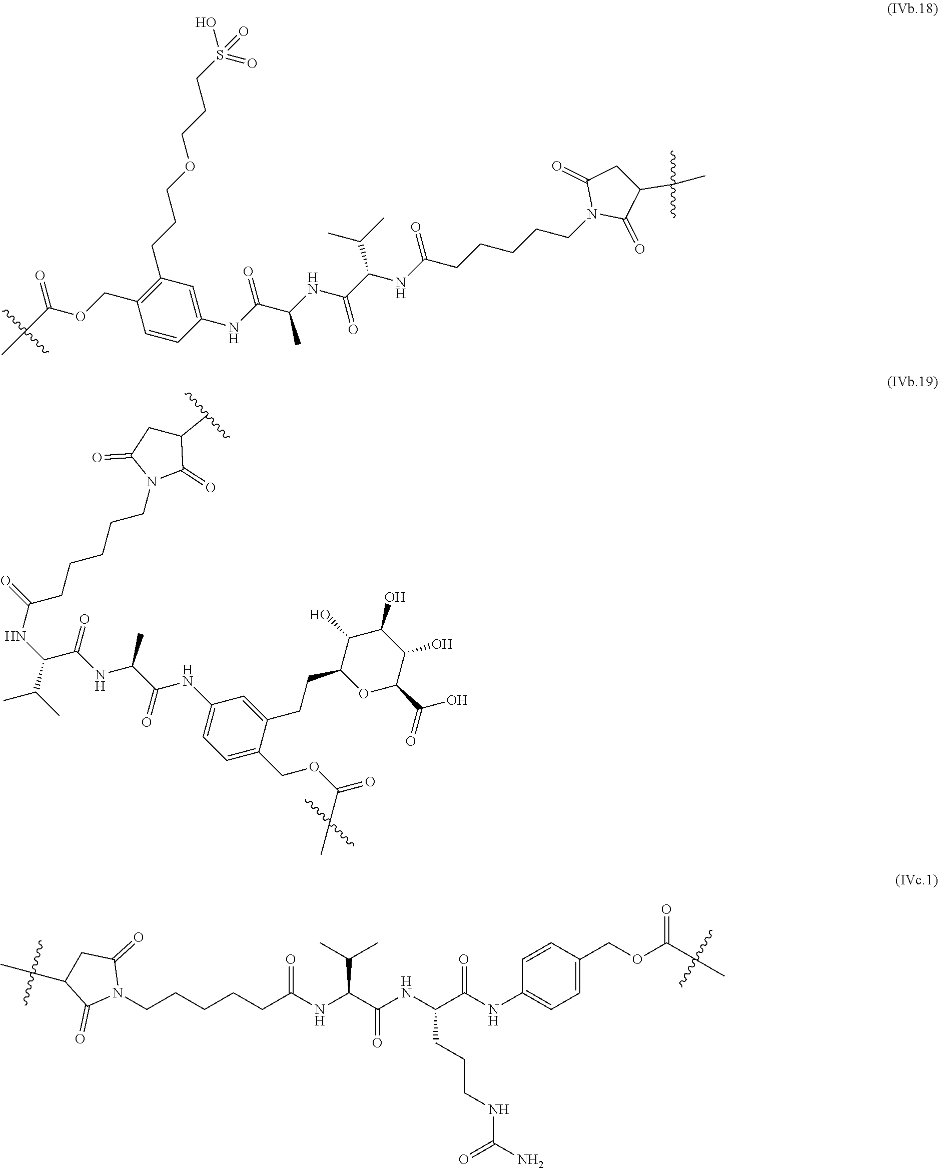 [SMILES: *C(=O)OCc1ccc(NC(=O)[C@H](C)NC(=O)[C@@H](NC(=O)CCCCCN2C(=O)CC(*)C2=O)C(C)C)cc1CCCOCCCS(=O)(=O)O ; *C(=O)OCc1ccc(NC(=O)[C@H](C)NC(=O)[C@@H](NC(=O)CCCCCN2C(=O)CC(*)C2=O)C(C)C)cc1CC[C@@H]1O[C@H](C(=O)O)[C@@H](O)[C@H](O)[C@H]1O ; *C(=O)OCc1ccc(NC(=O)[C@H](CCCNC(N)=O)NC(=O)[C@@H](NC(=O)CCCCCN2C(=O)CC(*)C2=O)C(C)C)cc1]

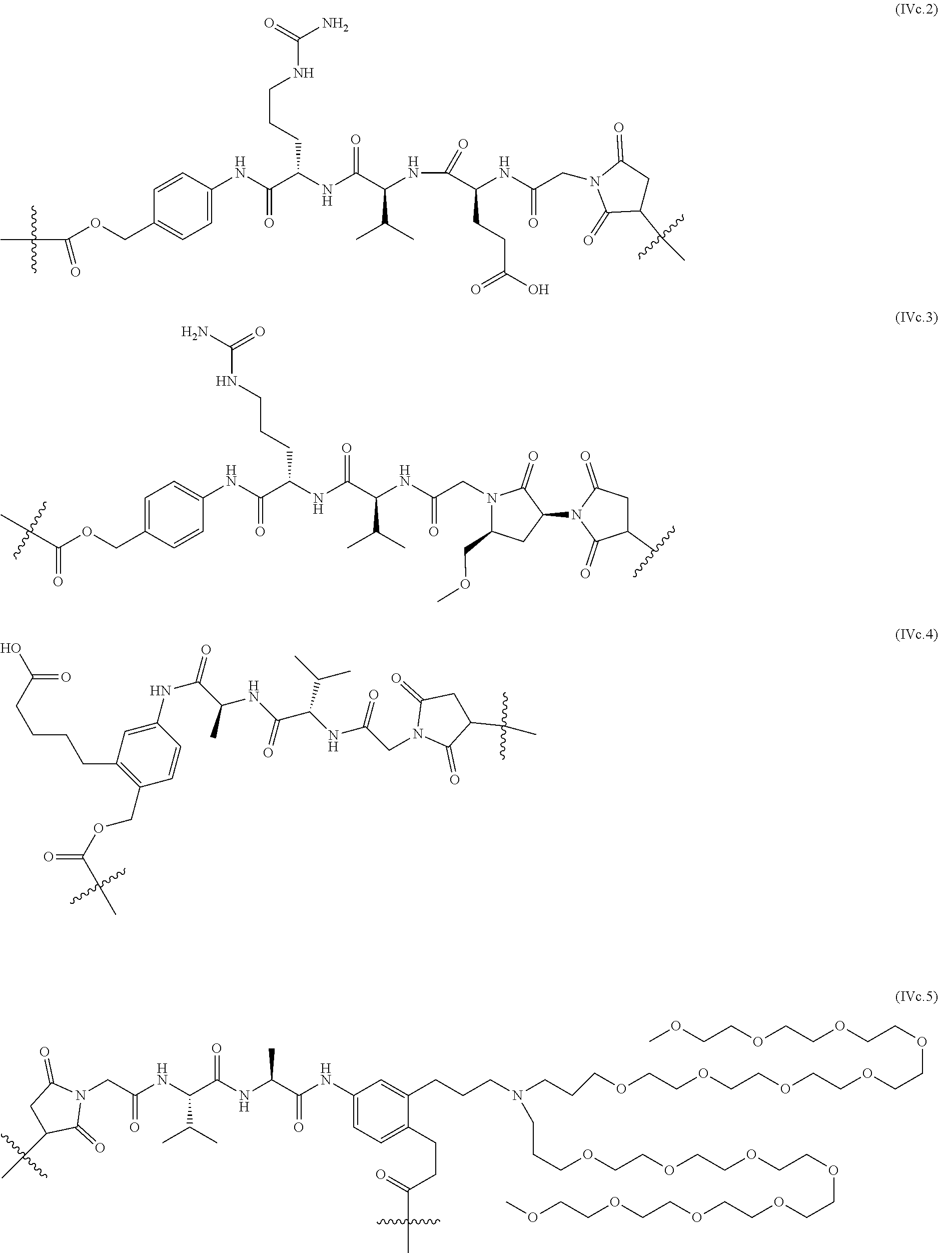 [SMILES: *C(=O)OCc1ccc(NC(=O)[C@H](CCCNC(N)=O)NC(=O)[C@@H](NC(=O)[C@H](CCC(=O)O)NC(=O)CN2C(=O)CC(*)C2=O)C(C)C)cc1 ; *C(=O)OCc1ccc(NC(=O)[C@H](CCCNC(N)=O)NC(=O)[C@@H](NC(=O)CN2C(=O)[C@@H](N3C(=O)CC(*)C3=O)C[C@H]2COC)C(C)C)cc1 ; *C(=O)OCc1ccc(NC(=O)[C@H](C)NC(=O)[C@@H](NC(=O)CN2C(=O)CC(*)C2=O)C(C)C)cc1CCCCC(=O)O ; *C(=O)CCc1ccc(NC(=O)[C@H](C)NC(=O)[C@@H](NC(=O)CN2C(=O)CC(*)C2=O)C(C)C)cc1CCCN(CCCOCCOCCOCCOCCOCCOCCOCCOC)CCCOCCOCCOCCOCCOCCOCCOCCOC]

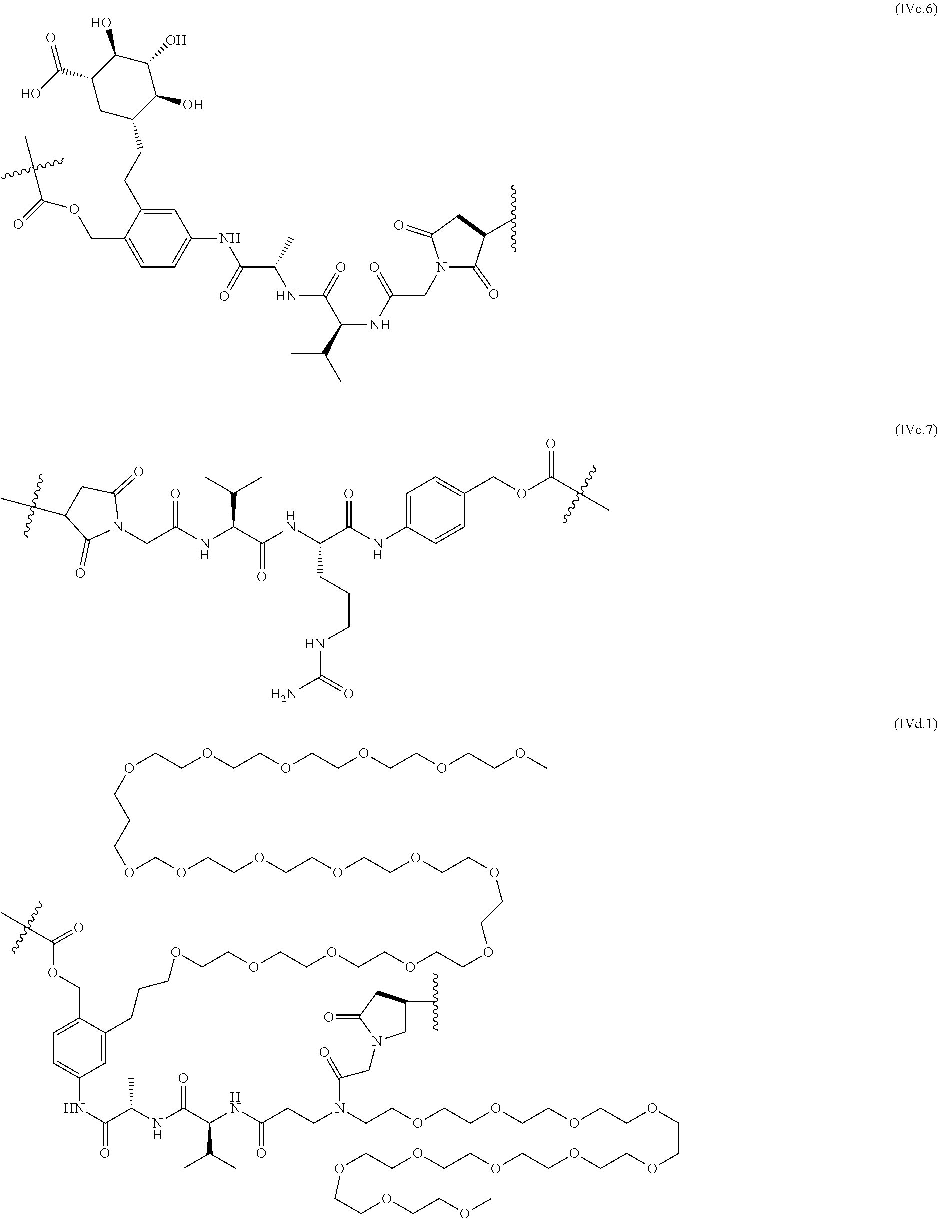 [SMILES: *C(=O)OCc1ccc(NC(=O)[C@H](C)NC(=O)[C@@H](NC(=O)CN2C(=O)C[C@@H](*)C2=O)C(C)C)cc1CC[C@@H]1C[C@H](C(=O)O)[C@@H](O)[C@H](O)[C@H]1O ; *C(=O)OCc1ccc(NC(=O)[C@H](CCCNC(N)=O)NC(=O)[C@@H](NC(=O)CN2C(=O)CC(*)C2=O)C(C)C)cc1 ; *C(=O)OCc1ccc(NC(=O)[C@H](C)NC(=O)[C@@H](NC(=O)CCN(CCOCCOCCOCCOCCOCCOCCOCCOCCOCCOCCOC)C(=O)CN2C[C@H](*)CC2=O)C(C)C)cc1CCCOCCOCCOCCOCCOCCOCCOCCOCCOCCOCOCCCOCCOCCOCCOCCOCCOC]

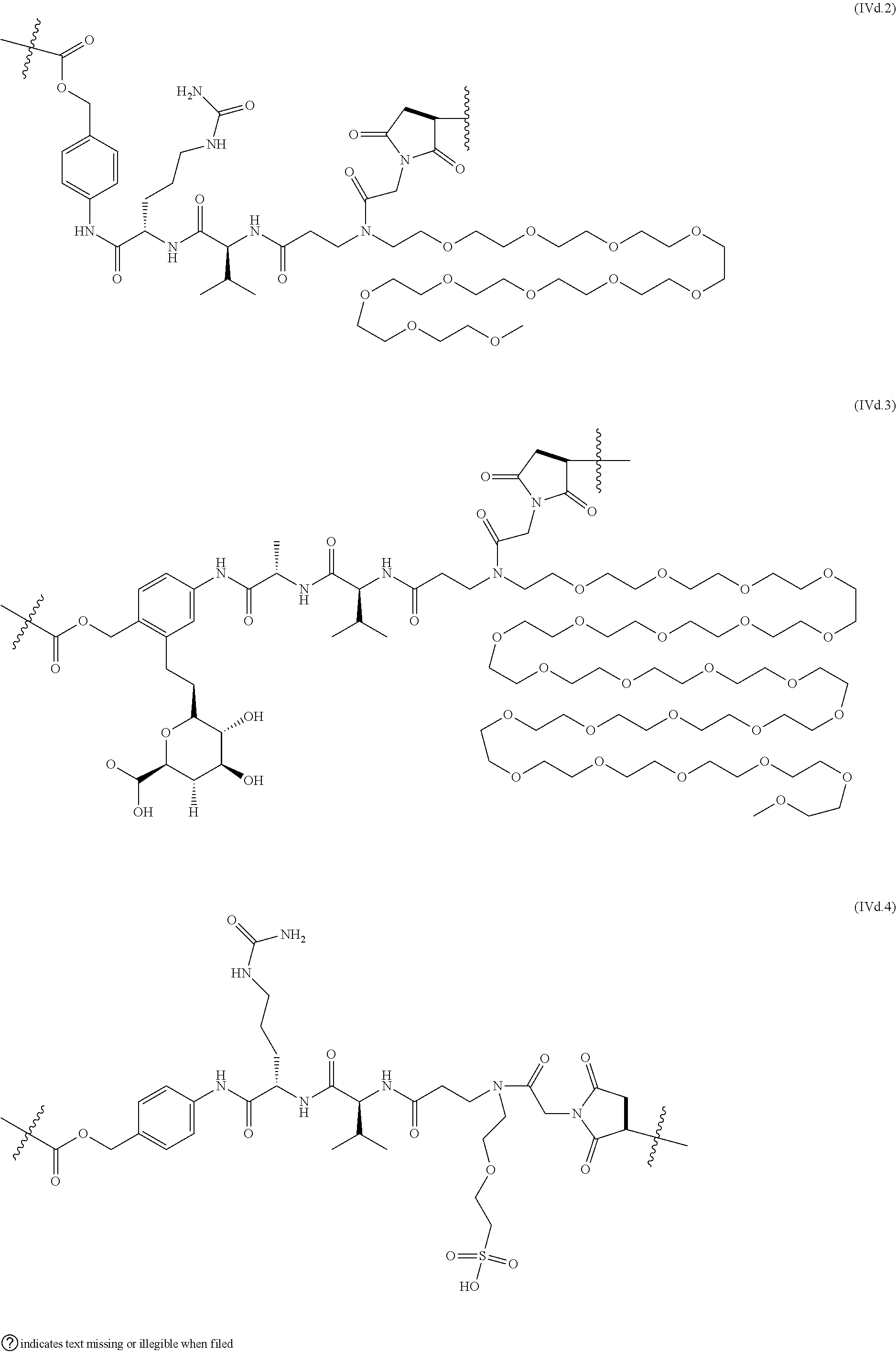

wherein one of [symbol] represents the point of attachment of the linker (L³) to the inhibitor.

The cleavable linker can contain an enzymatically cleavable sugar moiety, for example, a linker comprising structural formula (Va), (Vb), (Vc), (Vd), or (Ve):

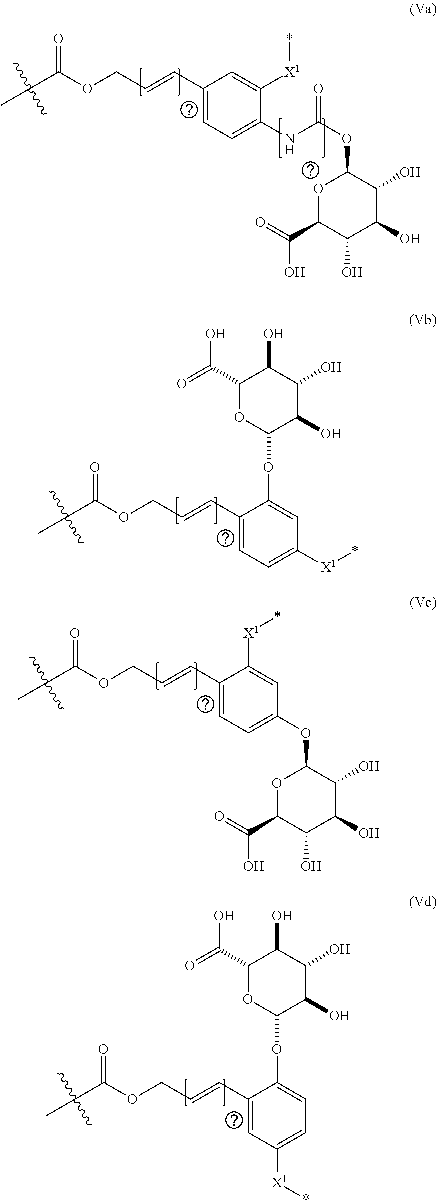

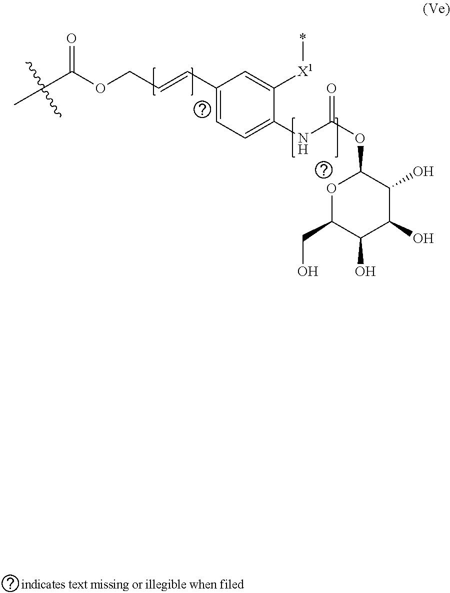

or a salt thereof, wherein: q is 0 or 1; r is 0 or 1; X¹ is CH₂, O or NH; [symbol] represents the point of attachment of the linker to the TGFβR1 inhibitor; and * represents the point of attachment to the remainder of the conjugate.

Exemplary embodiments of connector regions or connector segments according to structural formula (Va) that may be included in the conjugates described herein can include the incorporated moieties from the structures illustrated below, where the skilled practitioner would understand that, when linked within the conjugate, the maleimide in each structure will be in its linked form, i.e., a succinimide moiety

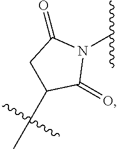 [SMILES: *C1CC(=O)N(*)C1=O]

—S—CH═CH₂ in each structure will be in its linked form i.e.,

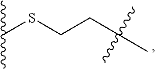 [SMILES: *CCS*]

and —SO₂—CH═CH₂ in will be in its linked form, i.e.,

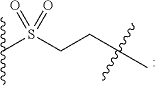

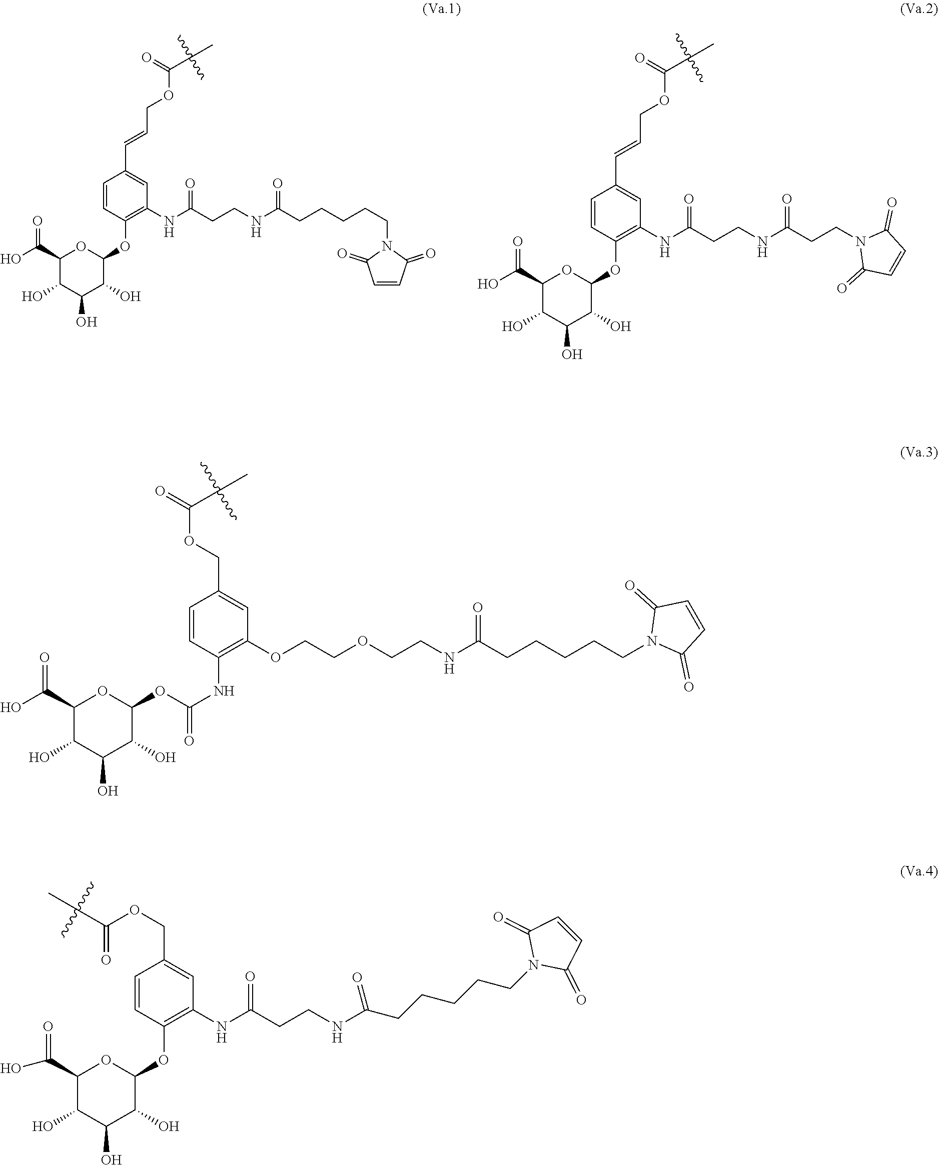

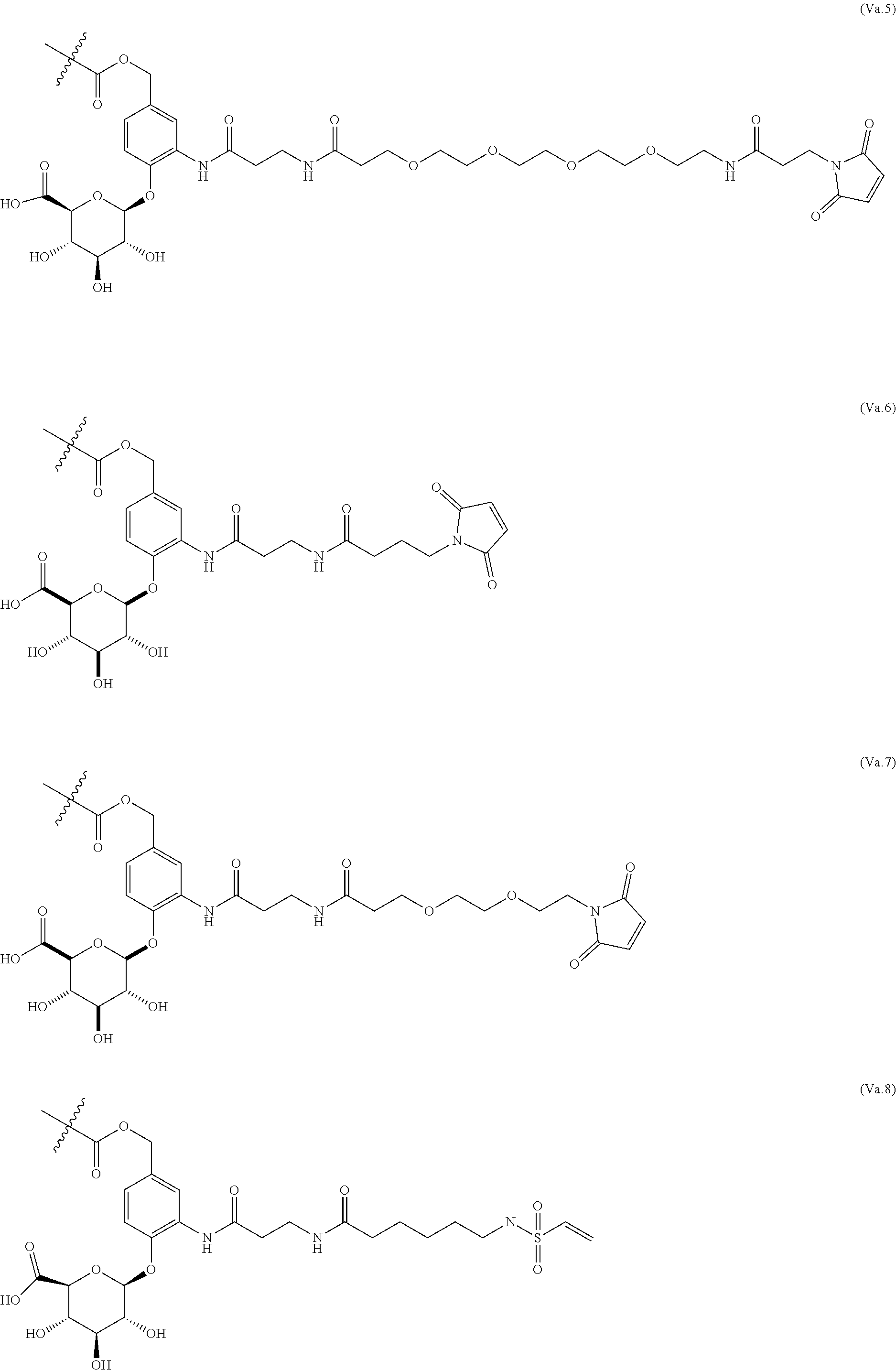

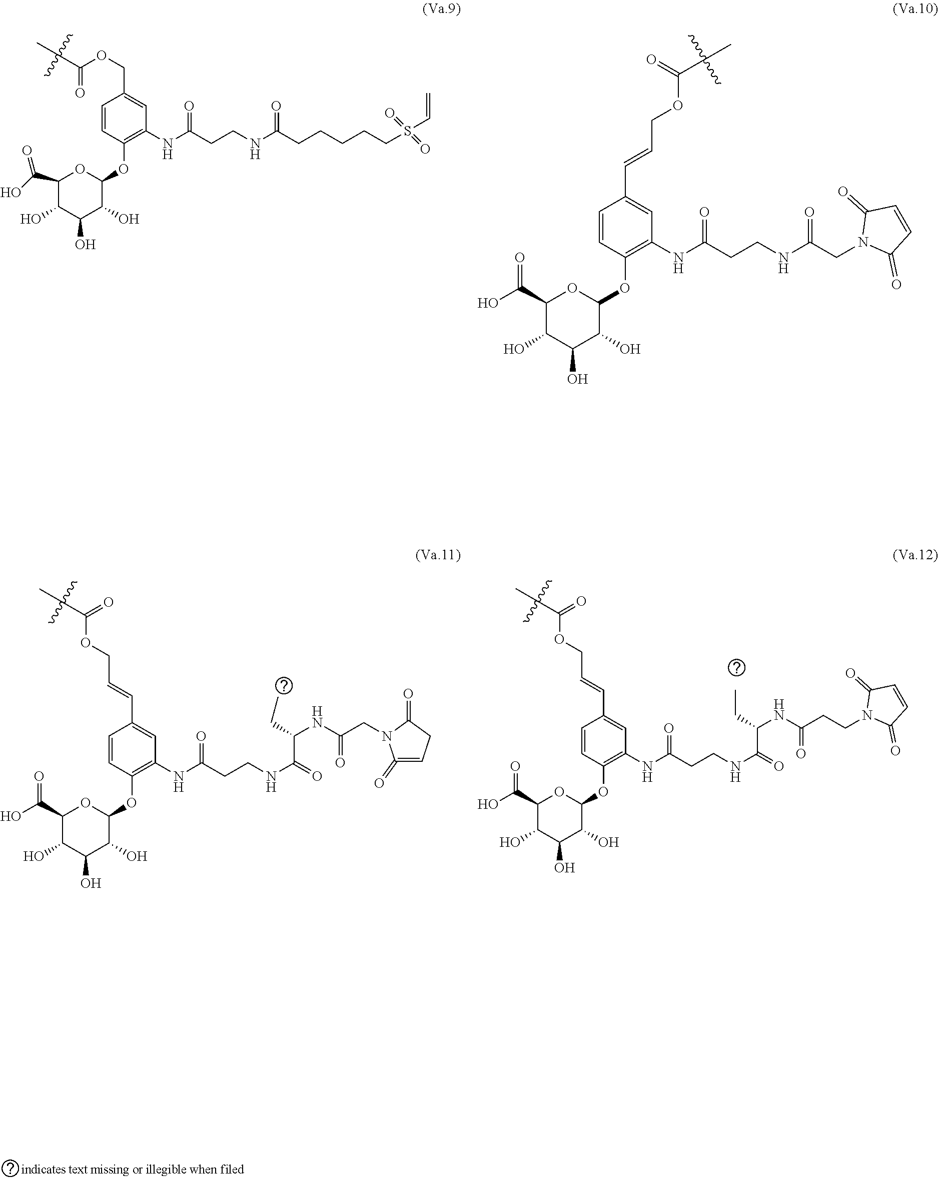

wherein [symbol] represents the point of attachment of the linker (L³) to the inhibitor.

Exemplary embodiments of connector regions or connector segments according to structural formula (Vb) that may be included in the conjugates described herein include the structures illustrated below, where the maleimide in each structure is replaced with a succinimide moiety

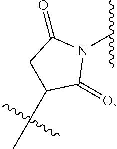 [SMILES: *C1CC(=O)N(*)C1=O]

in the conjugate.

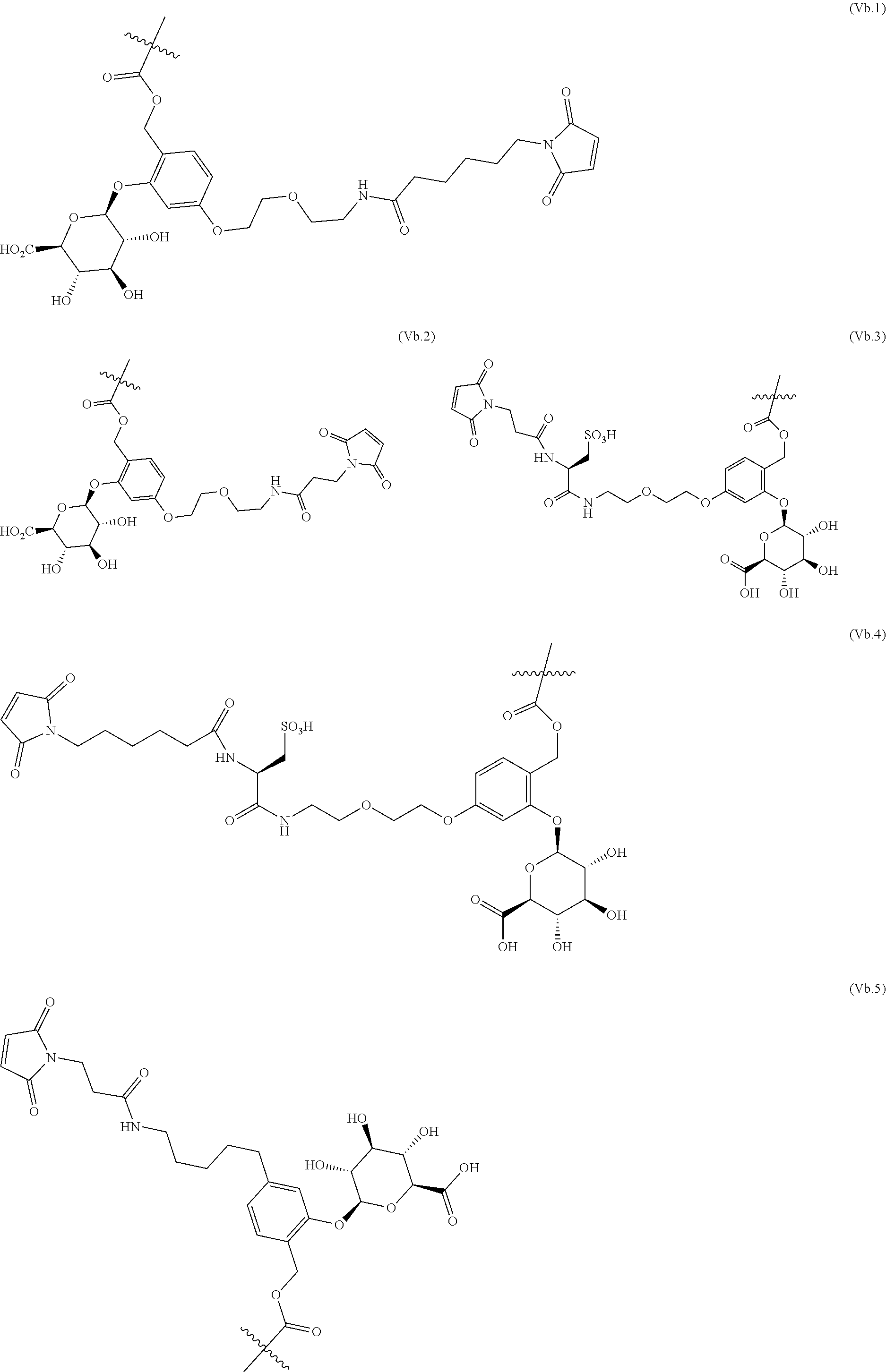

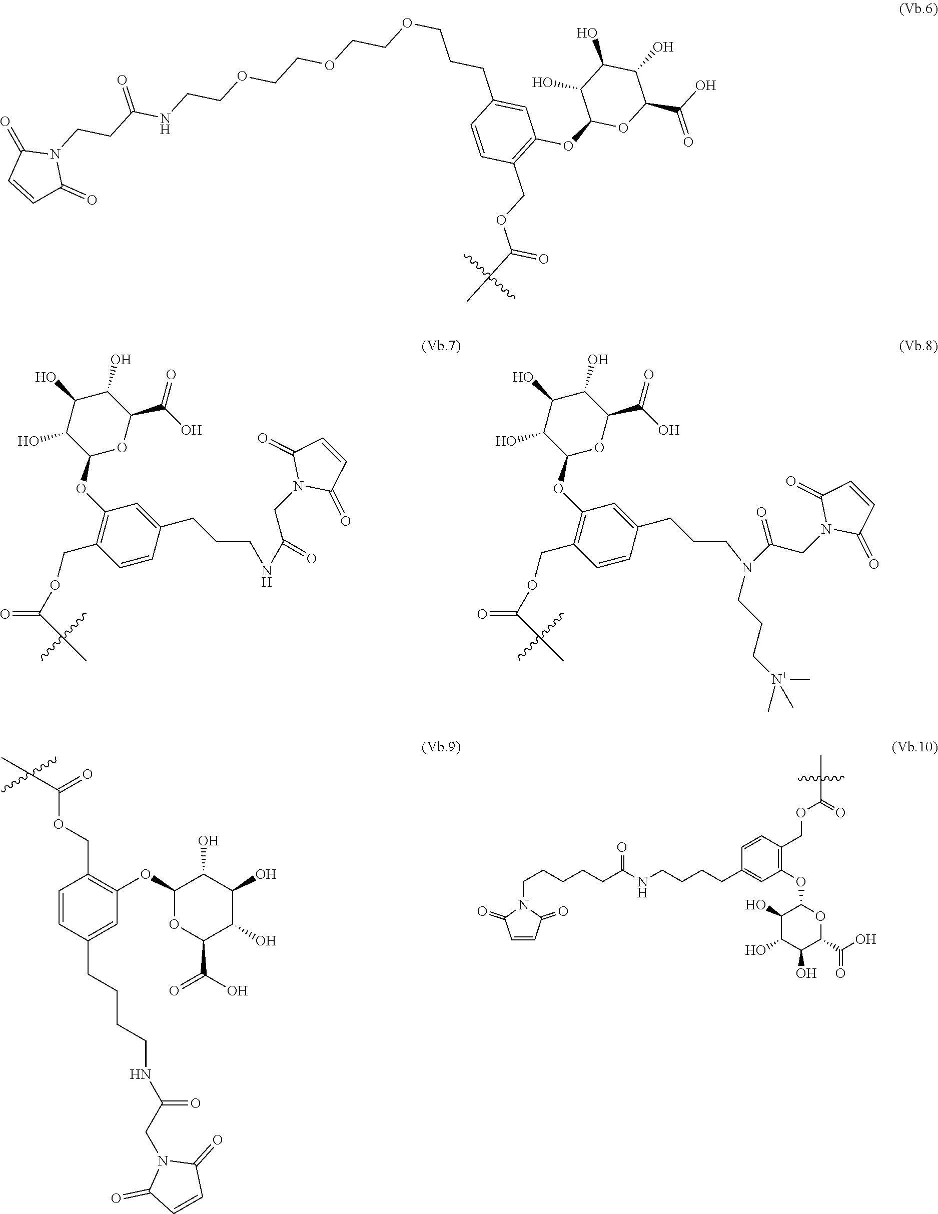 [SMILES: *C(=O)OCc1ccc(CCCOCCOCCOCCNC(=O)CCN2C(=O)C=CC2=O)cc1O[C@@H]1O[C@H](C(=O)O)[C@@H](O)[C@H](O)[C@H]1O ; *C(=O)OCc1ccc(CCCNC(=O)CN2C(=O)C=CC2=O)cc1O[C@@H]1O[C@H](C(=O)O)[C@@H](O)[C@H](O)[C@H]1O ; *C(=O)OCc1ccc(CCCN(CCC[N+](C)(C)C)C(=O)CN2C(=O)C=CC2=O)cc1O[C@@H]1O[C@H](C(=O)O)[C@@H](O)[C@H](O)[C@H]1O ; *C(=O)OCc1ccc(CCCCNC(=O)CN2C(=O)C=CC2=O)cc1O[C@@H]1O[C@H](C(=O)O)[C@@H](O)[C@H](O)[C@H]1O ; *C(=O)OCc1ccc(CCCCNC(=O)CCCCCN2C(=O)C=CC2=O)cc1O[C@@H]1O[C@H](C(=O)O)[C@@H](O)[C@H](O)[C@H]1O]

wherein one of [symbol] represents the point of attachment of the linker (L³) to the inhibitor.

Exemplary embodiments of connector regions or connector segments according to structural formula (Vc) that may be included in the conjugates described herein include the linkers illustrated below, where the maleimide in each structure is replaced with a succinimide moiety in

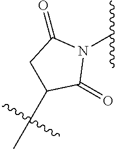 [SMILES: *C1CC(=O)N(*)C1=O]

the conjugate:

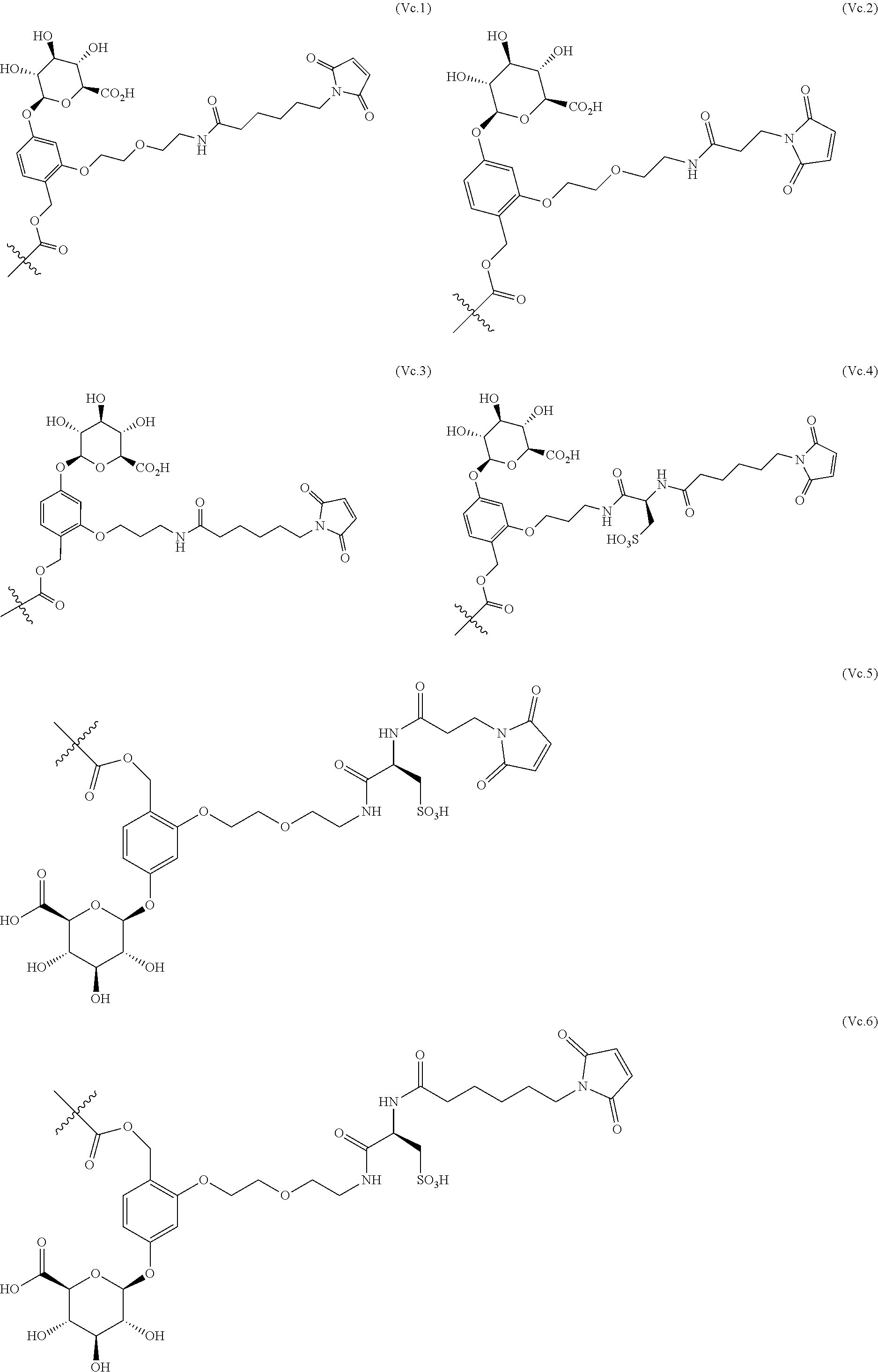

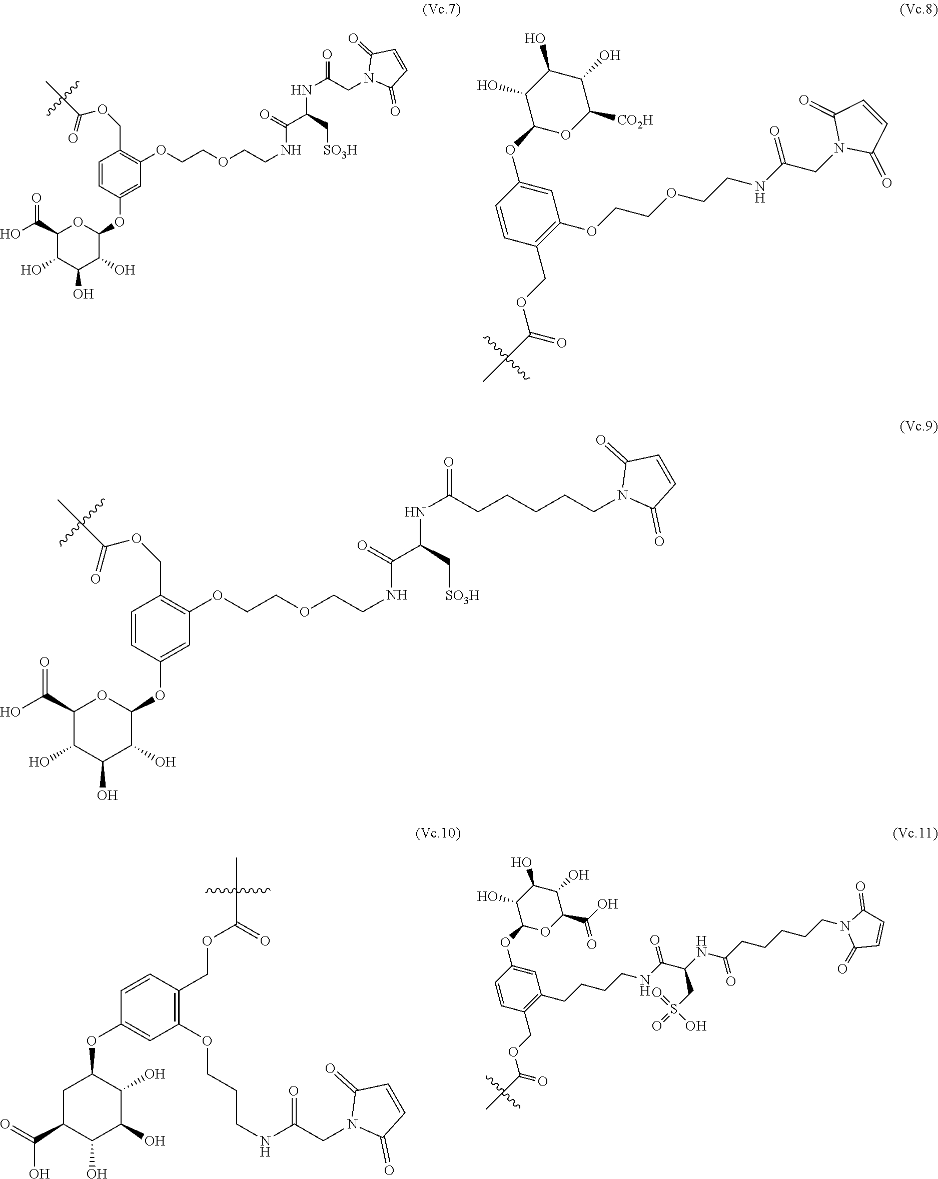

wherein one of [symbol] represents the point of attachment of the linker (L³) to the inhibitor.

Exemplary embodiments of connector regions or connector segments according to structural formula (Vd) that may be included in the conjugates described herein include the structures illustrated below, where the maleimide in each structure is replaced with a succinimide moiety

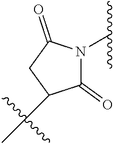 [SMILES: *C1CC(=O)N(*)C1=O]

in the conjugate:

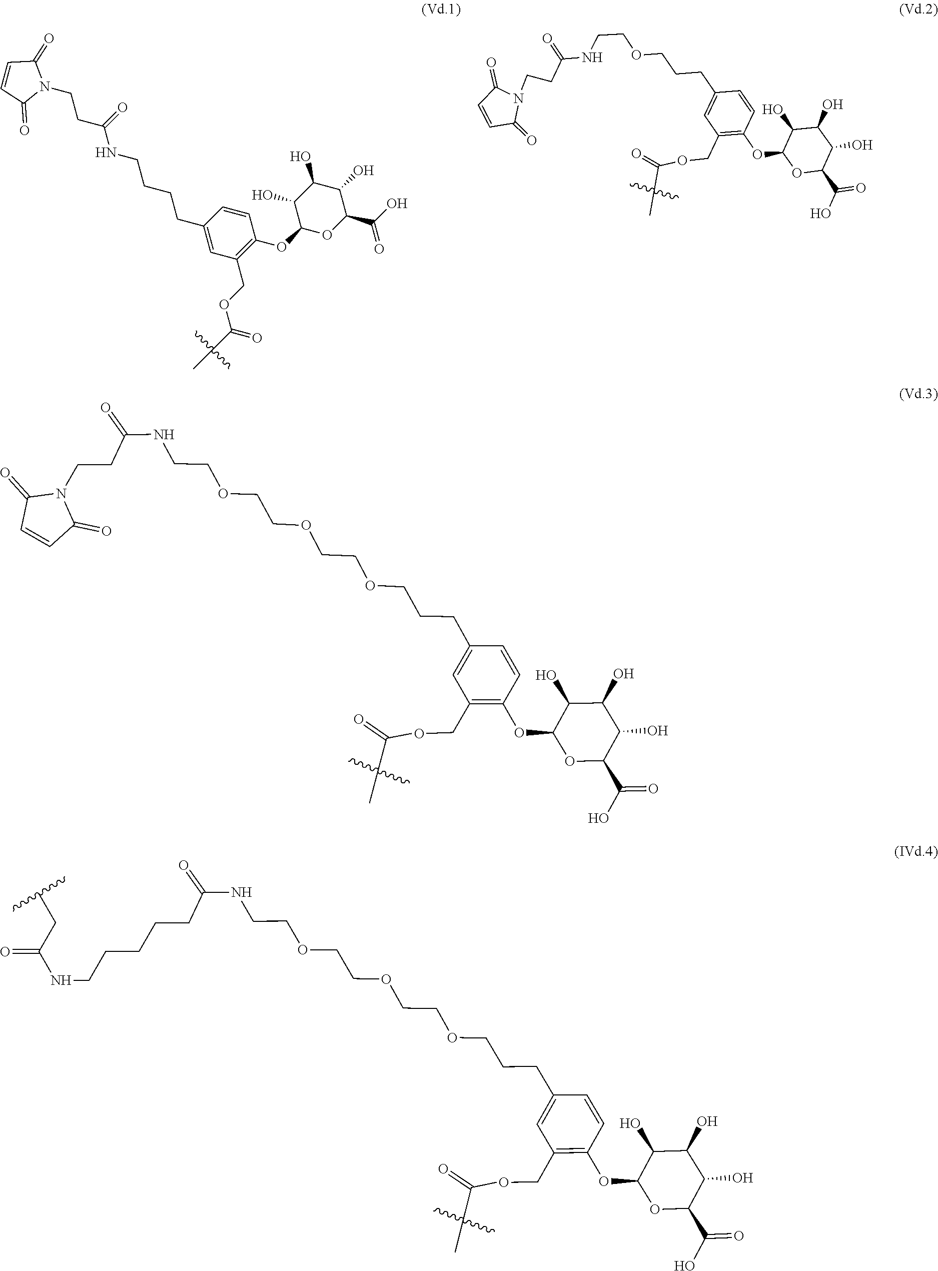 [SMILES: *C(=O)OCc1cc(CCCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCNC(=O)CCN2C(=O)C=CC2=O)ccc1O[C@@H]1O[C@H](C(=O)O)[C@@H](O)[C@H](O)[C@@H]1O ; *C(=O)OCc1cc(CCCOCCOCCOCCNC(=O)CCN2C(=O)C=CC2=O)ccc1O[C@@H]1O[C@H](C(=O)O)[C@@H](O)[C@H](O)[C@@H]1O ; *CC(=O)NCCCCCC(=O)NCCOCCOCCOCCCc1ccc(O[C@@H]2O[C@H](C(=O)O)[C@@H](O)[C@H](O)[C@@H]2O)c(COC(*)=O)c1]

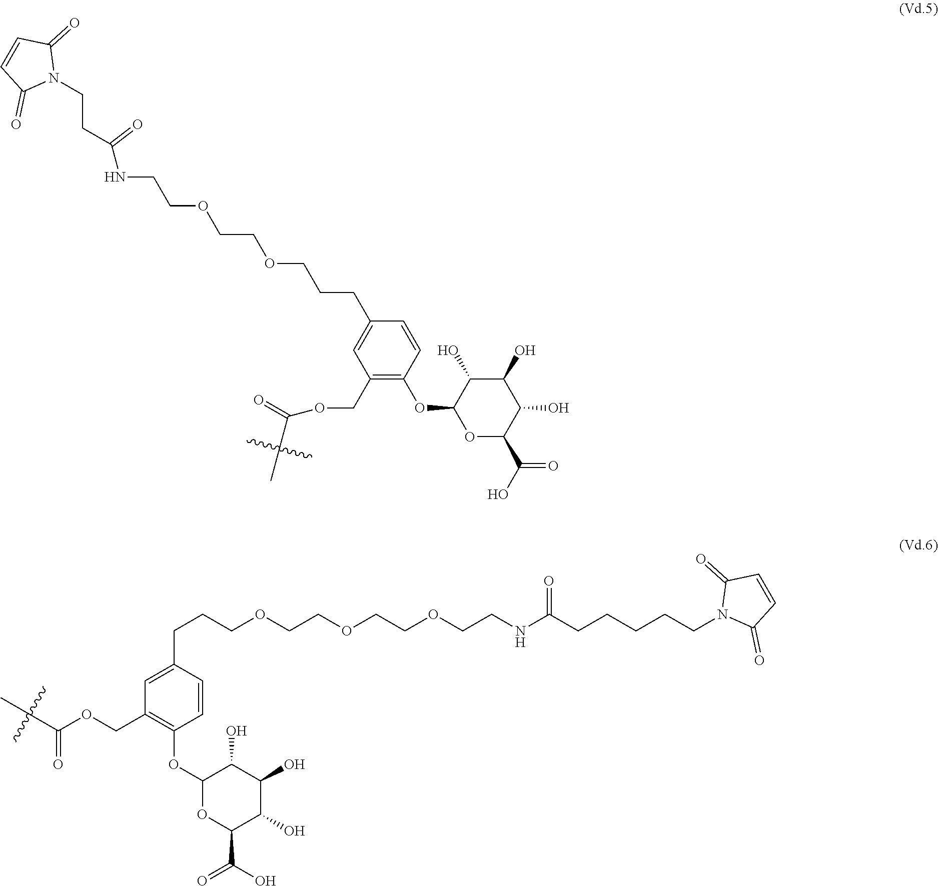 [SMILES: *C(=O)OCc1cc(CCCOCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCCCCN2C(=O)C=CC2=O)ccc1OC1O[C@H](C(=O)O)[C@@H](O)[C@H](O)[C@H]1O]

wherein one of [symbol] represents the point of attachment of the linker (L³) to the inhibitor.

Exemplary embodiments of connector regions or connector segments according to structural formula (Ve) that may be included in the conjugates described herein include the structures illustrated below, where the maleimide in each structure is replaced with a succinimide moiety

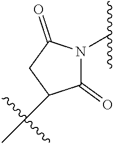 [SMILES: *C1CC(=O)N(*)C1=O]

in the conjugate.

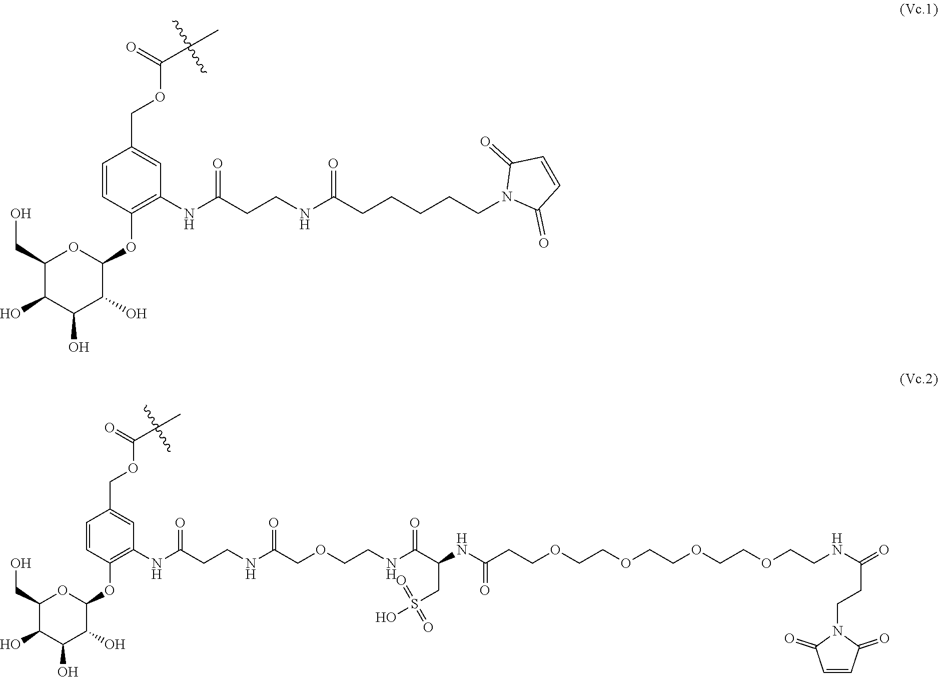 [SMILES: *C(=O)OCc1ccc(O[C@@H]2O[C@H](CO)[C@H](O)[C@H](O)[C@H]2O)c(NC(=O)CCNC(=O)CCCCCN2C(=O)C=CC2=O)c1 ; *C(=O)OCc1ccc(O[C@@H]2O[C@H](CO)[C@H](O)[C@H](O)[C@H]2O)c(NC(=O)CCNC(=O)COCCNC(=O)[C@H](CS(=O)(=O)O)NC(=O)CCOCCOCCOCCOCCNC(=O)CCN2C(=O)C=CC2=O)c1]

wherein one of [symbol] represents the point of attachment of the linker (L³) to the inhibitor.

Although cleavable linkers can provide certain advantages, the connector regions in the conjugates described herein need not include cleavable linkers. For non-cleavable linkers, the TGFβR1 inhibitor release may not depend on the differential properties between the plasma and some cytoplasmic compartments.

The linker can be non-cleavable in vivo, for example, a linker according to the formulations below:

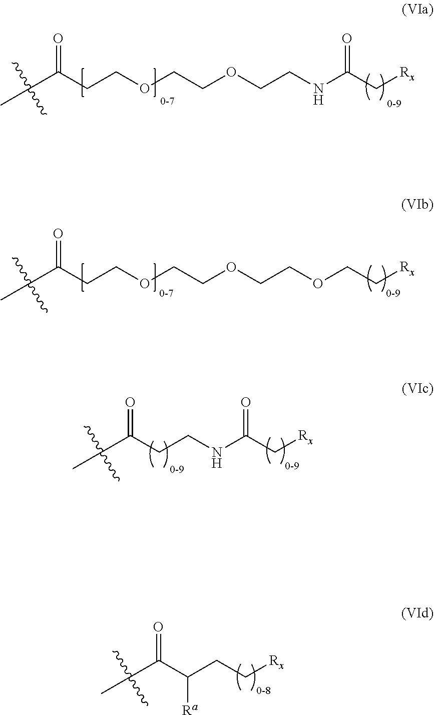

or salts thereof, wherein:

R^{a} is selected from hydrogen, alkyl, sulfonate and methyl sulfonate;

R^{x} is a moiety that covalently links the connector to the rest of the conjugate, such as a bond, a succinimide moiety, or a hydrolyzed succinimide moiety; and

[symbol] represents the point of attachment of the linker to the rest of the conjugate.

Exemplary embodiments of connector regions or connector segments according to structural formula (VIa)-(VId) that may be included in the conjugates described herein include the structures illustrated below, where the maleimide in each structure is replaced with a succinimide moiety

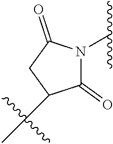 [SMILES: *C1CC(=O)N(*)C1=O]

in the conjugate and —SO₂—CH═CH₂ in each structure is replaced with

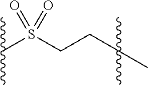 [SMILES: *CCS(*)(=O)=O]

in the conjugate:

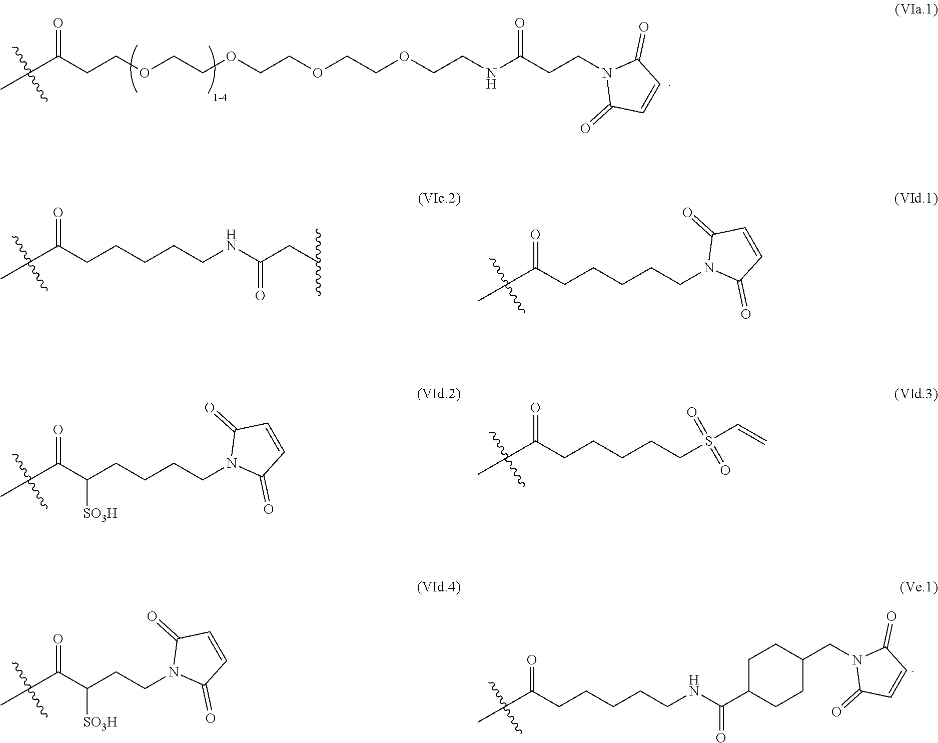

where [symbol] represents the point of attachment of the linker to the rest of the conjugate.

Attachment groups that are used to attach the linkers in a conjugate can be electrophilic in nature and include, for example, maleimide groups, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used in accordance with the disclosure. Maleimide groups are frequently used in the preparation of conjugates because of their specificity for reacting with thiol groups.

A linker (L³) with a maleimide group may include an electron withdrawing groups such as, but not limited to, —C(O)R, ═O, —CN, —NO₂, —CX₃, —X, —COOR, —CONR₂, —COR, —COX, —SO₂R, —SO₂OR, —SO₂NHR, —SO₂NR₂, —PO₃R₂, —P(O)(CH₃)NHR, —NO, —NR₃⁺, —CR═CR₂, and —C≡CR, where each R is independently selected from H and C₁₋₆ alkyl and each X is independently selected from F, Br, Cl, and I. Self-stabilizing linkers may also include aryl, e.g., phenyl, or heteroaryl, e.g., pyridine, groups optionally substituted with electron withdrawing groups such as those described herein.

Examples of self-stabilizing linkers are provided in, e.g., U.S. Patent Publication No. 2013/0309256, the linkers of which are incorporated by reference herein. It will be understood that a self-stabilizing linker useful in conjunction with the compounds of the present disclosure may be equivalently described as unsubstituted maleimide-including linkers, thio-substituted succinimide-including linkers, or hydrolyzed, ring-opened thio-substituted succinimide-including linkers.

In certain embodiments, a linker of the disclosure (L³) comprises a stabilizing group selected from:

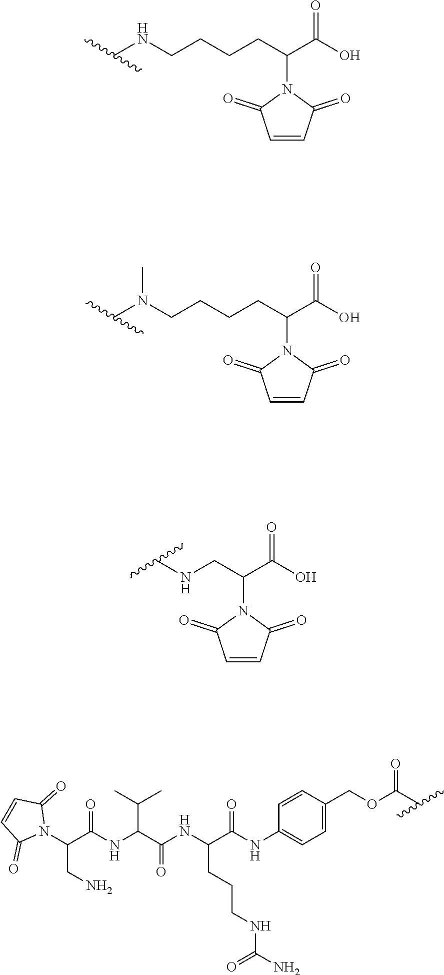 [SMILES: *NCCCCC(C(=O)O)N1C(=O)C=CC1=O ; *N(C)CCCCC(C(=O)O)N1C(=O)C=CC1=O ; *NCC(C(=O)O)N1C(=O)C=CC1=O ; *C(=O)OCc1ccc(NC(=O)C(CCCNC(N)=O)NC(=O)C(NC(=O)C(CN)N2C(=O)C=CC2=O)C(C)C)cc1]

In the scheme provided above, the bottom structure may be referred to as (maleimido)-DPR-Val-Cit-PAB, where DPR refers to diaminopropinoic acid, Val refers to valine, Cit refers to citrulline, and PAB refers to para-aminobenzylcarbonyl. [symbol] represent the point of attachment to an inhibitor as described herein.

The attachment moiety can contain the following structural formulas (VIIa), (VIIb), or (VIIc), where the maleimide in each structure is replaced with a succinimide moiety

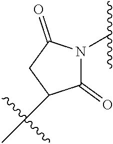 [SMILES: *C1CC(=O)N(*)C1=O]

in the conjugate:

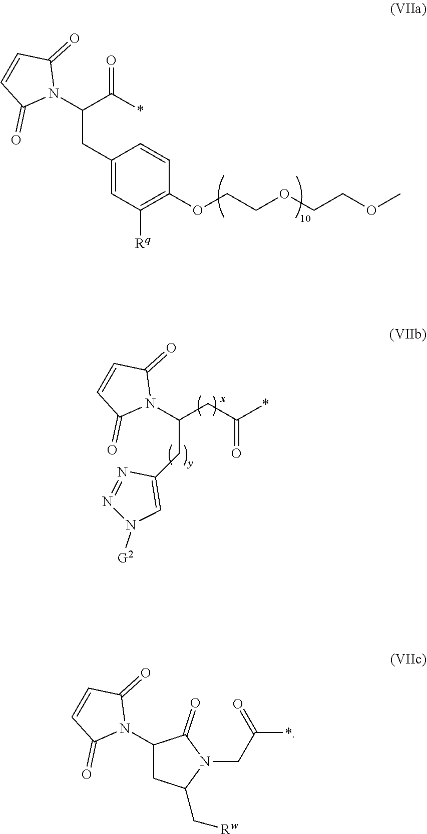

or salts thereof, wherein:

R^{q} is H or —O—(CH₂CH₂O)₁₁—CH₃;

x is 0 or 1;

y is 0 or 1;

G² is —CH₂CH₂CH₂SO₃H or —CH₂CH₂O—(CH₂CH₂O)₁₁—CH₃;

R^{w} is —O—CH₂CH₂SO₃H or —NH(CO)—CH₂CH₂O—(CH₂CH₂O)₁₂—CH₃; and

* represents the point of attachment to the remainder of the linker or conjugate.

Exemplary embodiments of connector regions or connector segments according to structural formula (VIIa) and (VIIb) that can be included in the conjugates described herein can include the structures illustrated below, where the maleimide in each structure is replaced with a succinimide moiety

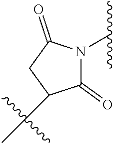 [SMILES: *C1CC(=O)N(*)C1=O]

in the conjugate:

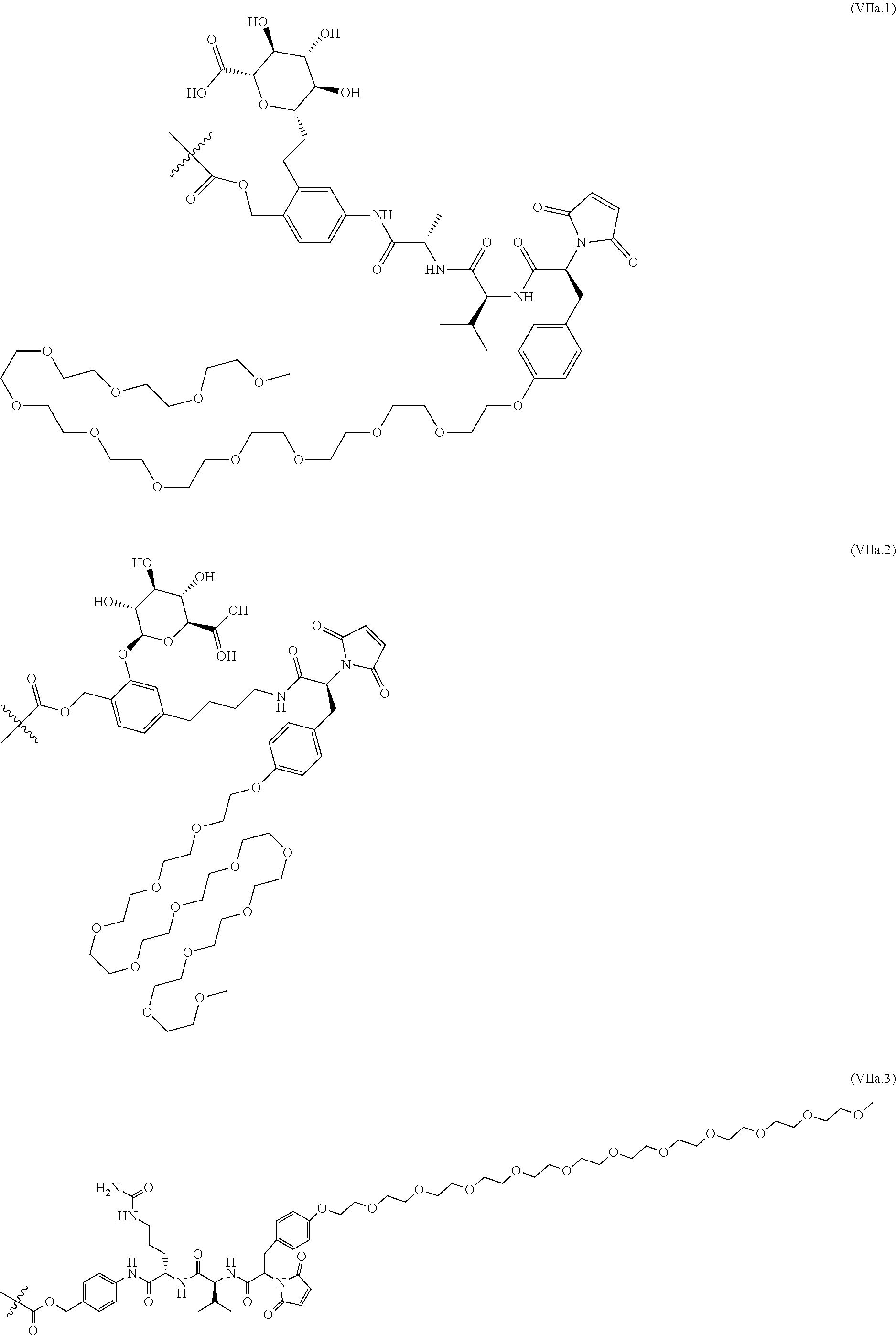

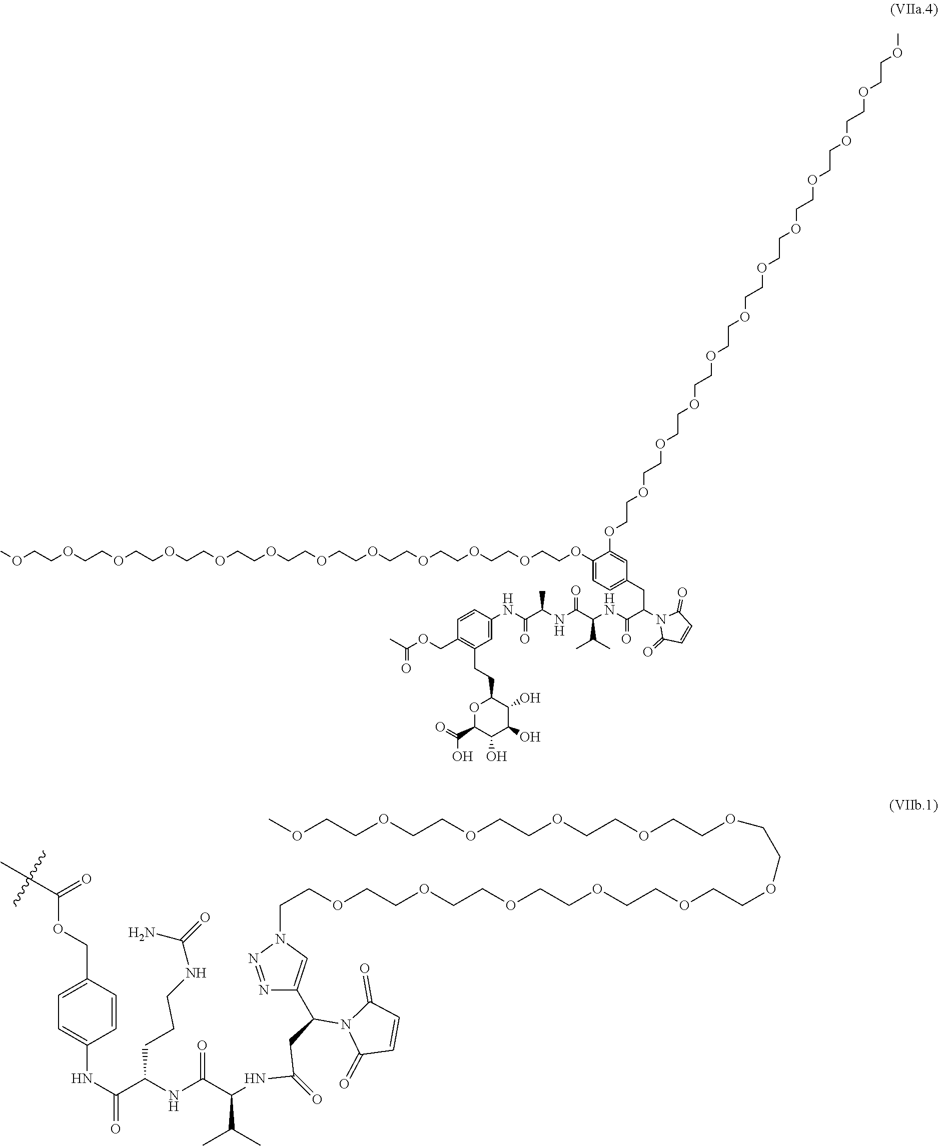

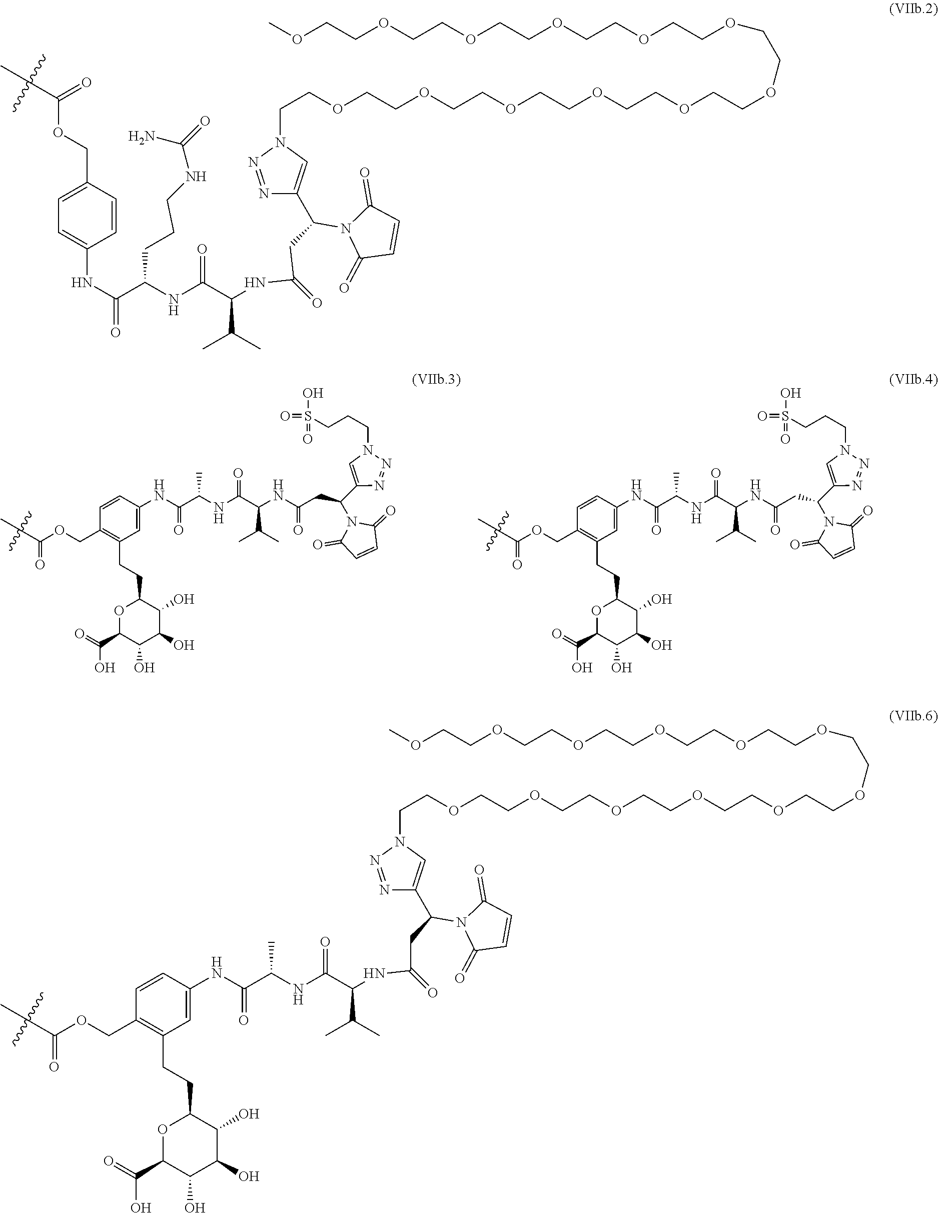

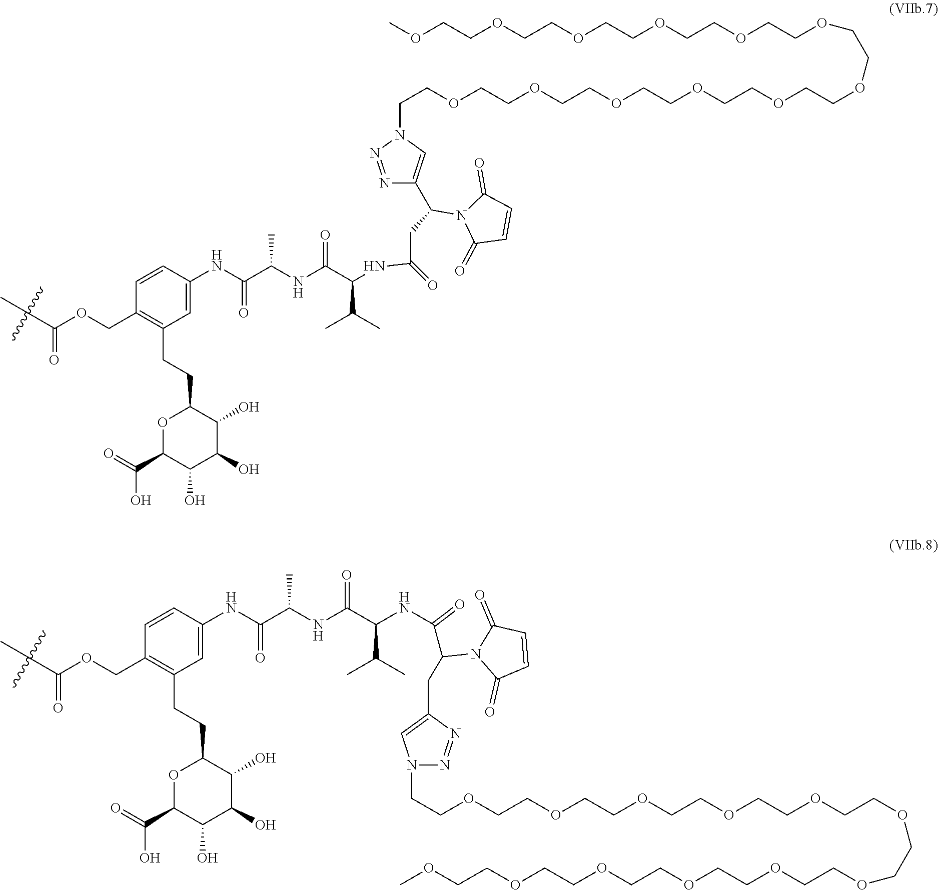

wherein [symbol] represents the point of attachment of the linker (L³) to the inhibitor.

Exemplary embodiments of connector regions or connector segments according to structural formula (VIIc) that can be included in the conjugates described herein can include the structures illustrated below, where the maleimide in each structure is replaced with a succinimide moiety

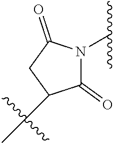 [SMILES: *C1CC(=O)N(*)C1=O]

in the conjugate:

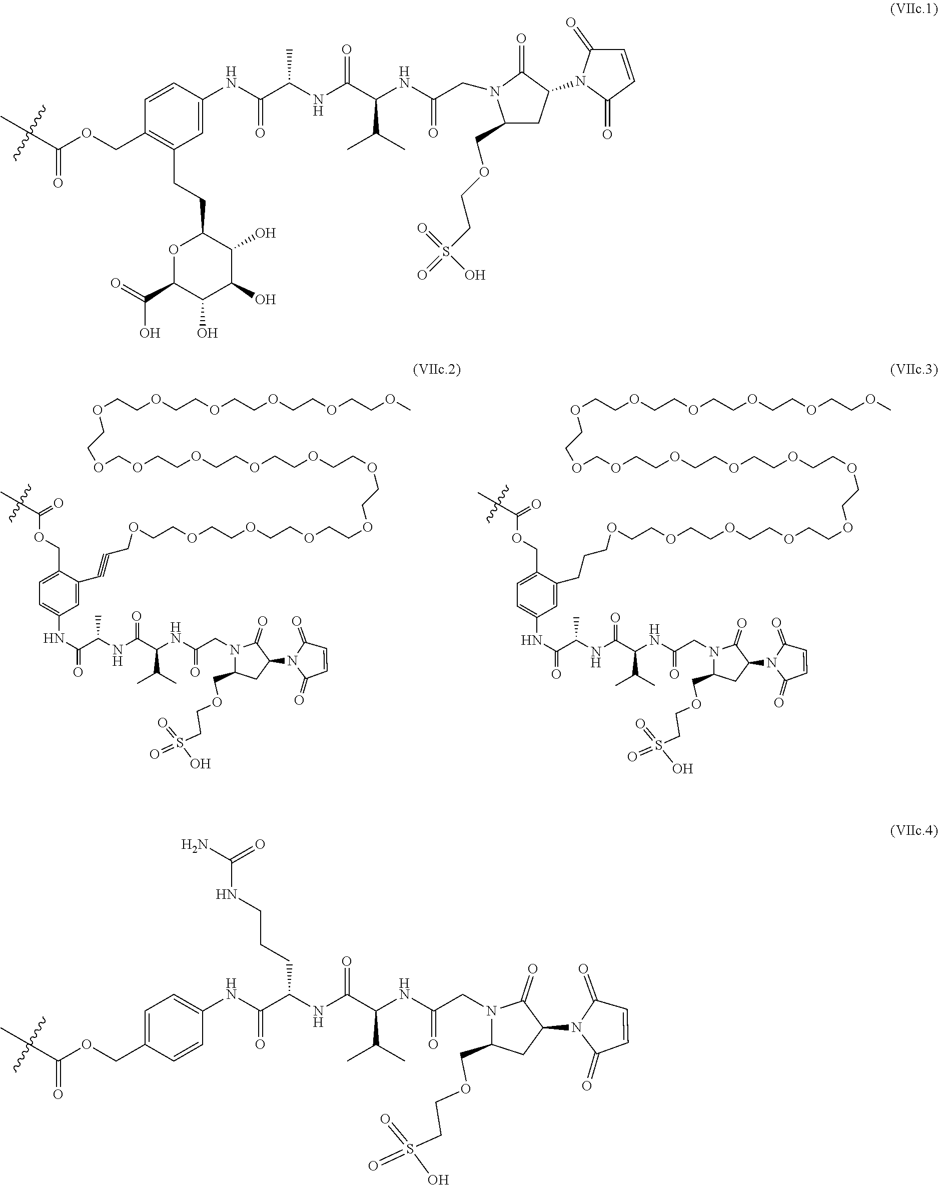

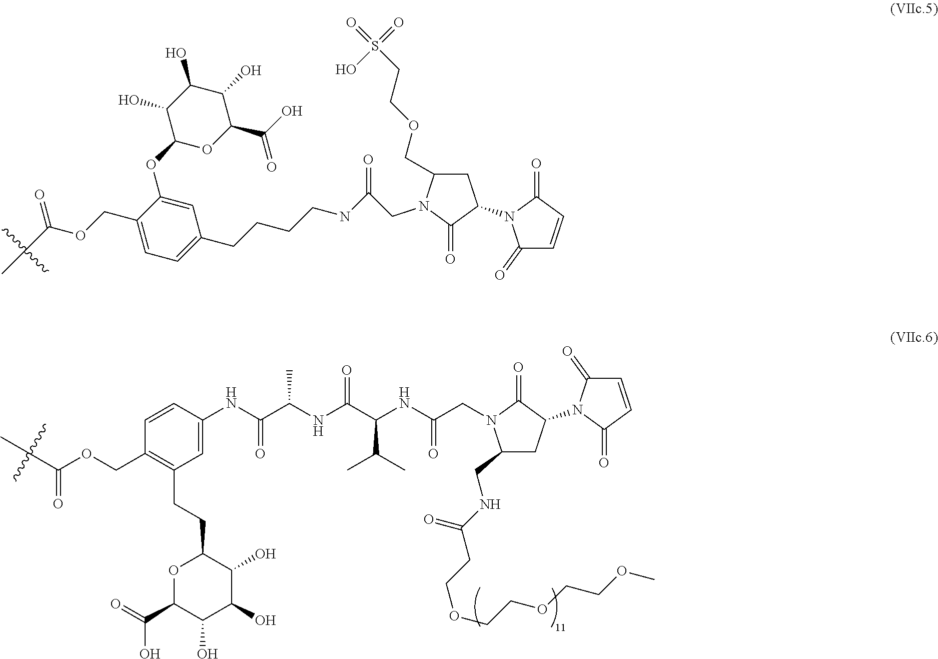

wherein [symbol] represents the point of attachment of the linker (L³) to the inhibitor.

In some embodiments, the linker or L³ comprises or is the following structure:

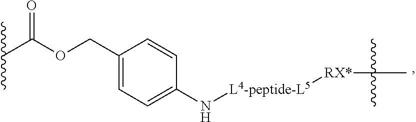

wherein.

L⁴ represents the C-terminus of the peptide;

L⁵ is selected from a bond, alkylene, and heteroalkylene, wherein L⁵ is optionally substituted with one or more groups independently selected from R³².

R³² is independently selected at each occurrence from halogen, —OH, —CN, —O-alkyl, —SH, ═O, ═S, —NH₂, —NO₂; and C₁₋₁₀ alkyl, C₂₋₁₀ alkenyl, C₂₋₁₀ alkynyl, each of which is optionally substituted with one or more substituents independently selected from halogen, —OH, —CN, —O-alkyl, —SH, ═O, ═S, —NH₂, —NO₂; and

RX* is a succinimido group, a hydrolyzed succinimido group, or a bond. In some embodiments, the peptide comprises Val-Cit or Val-Ala.

Some exemplary linkers (e.g., L³) are described in the following paragraphs. In some embodiments for an inhibitor wherein attachment of the linker is to a nitrogen of the inhibitor, the linker or -L³ is represented by a formula set forth in Table 1 below:

TABLE 1

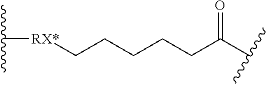

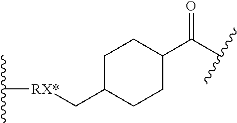

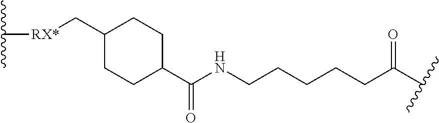

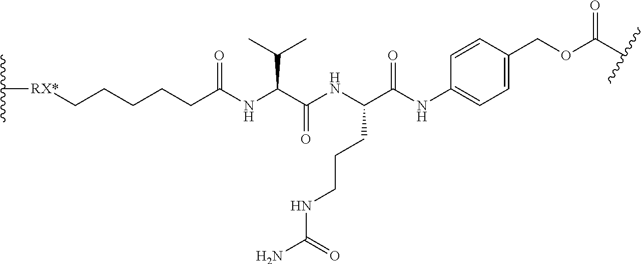

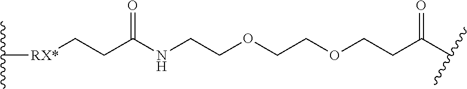

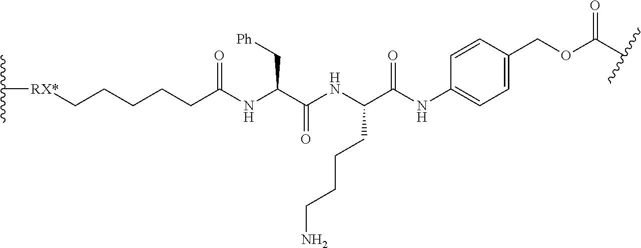

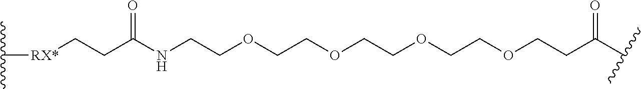

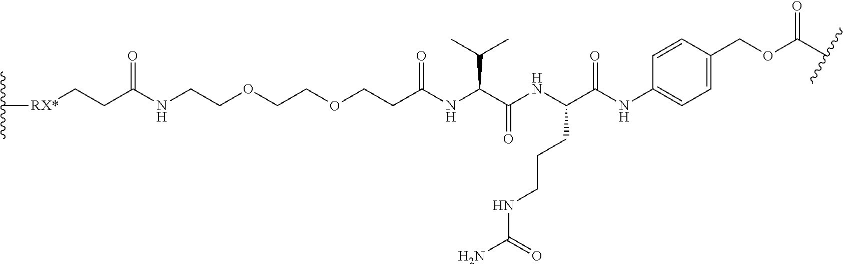

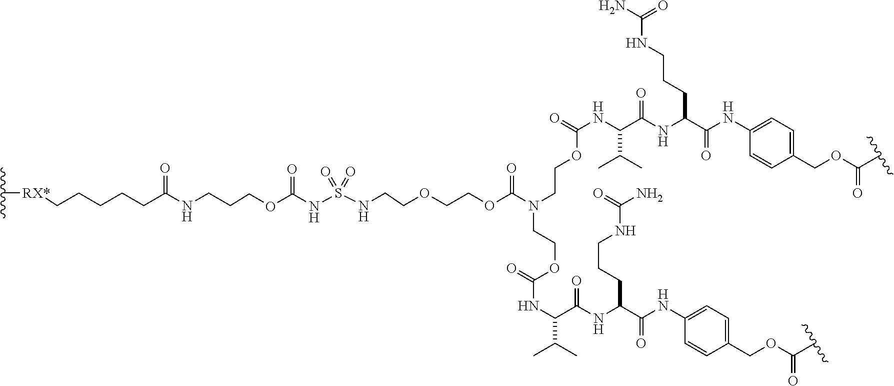

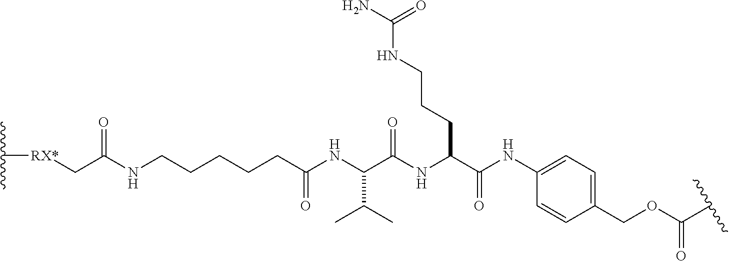

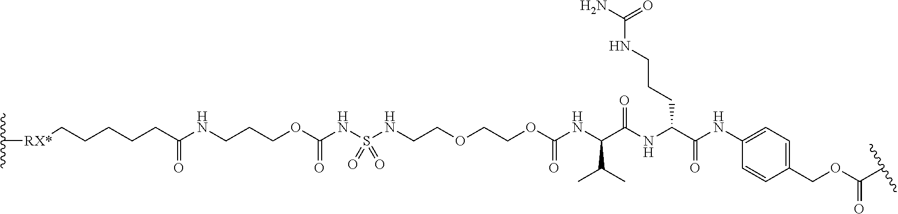

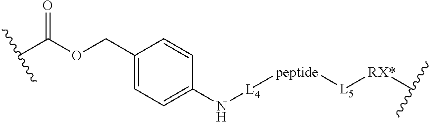

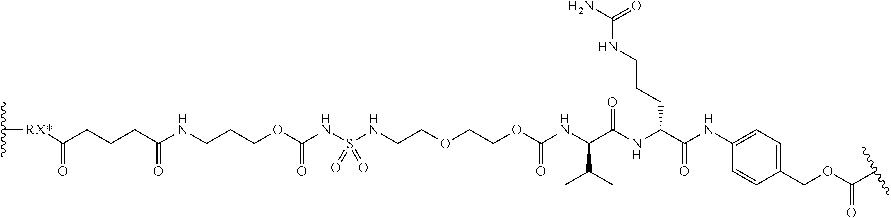

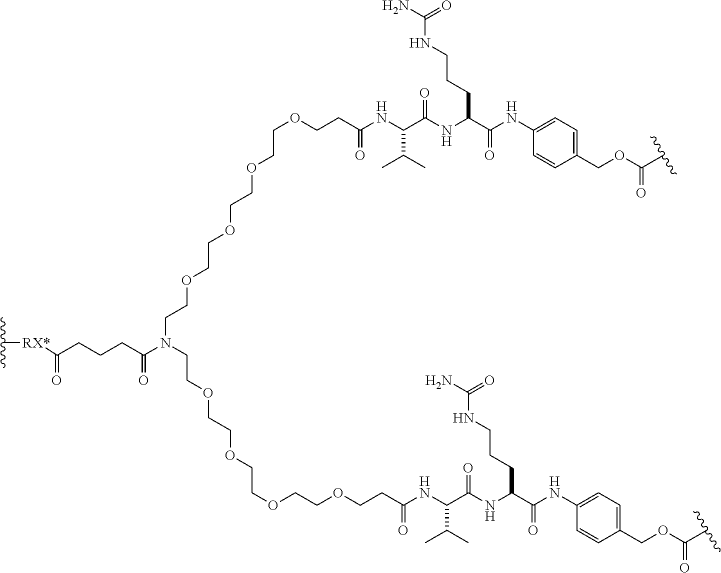

wherein

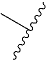

represents the point of attachment of the linker to the nitrogen of the TGFβR1 inhibitor;

RX* is a bond, a succinamide moiety, or a hydrolyzed succinamide moiety bound to a residue of the antibody construct, wherein

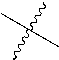

on RX* represents the point of attachment to the antibody construct; optionally wherein

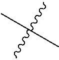

on RX* represents the point of attachment to a cysteine residue of the antibody construct;

L⁴ represents the C-terminus of the peptide; and

L⁵ is selected from a bond, alkylene, and heteroalkylene, wherein L⁵ is optionally substituted with one or more groups independently selected from R³⁰;

wherein each R³⁰ is independently selected from halogen, —OH, —CN, —O-alkyl, —SH, ═O, ═S, —NH₂, —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂.

In some aspects, the conjugate comprises one of the following structures:

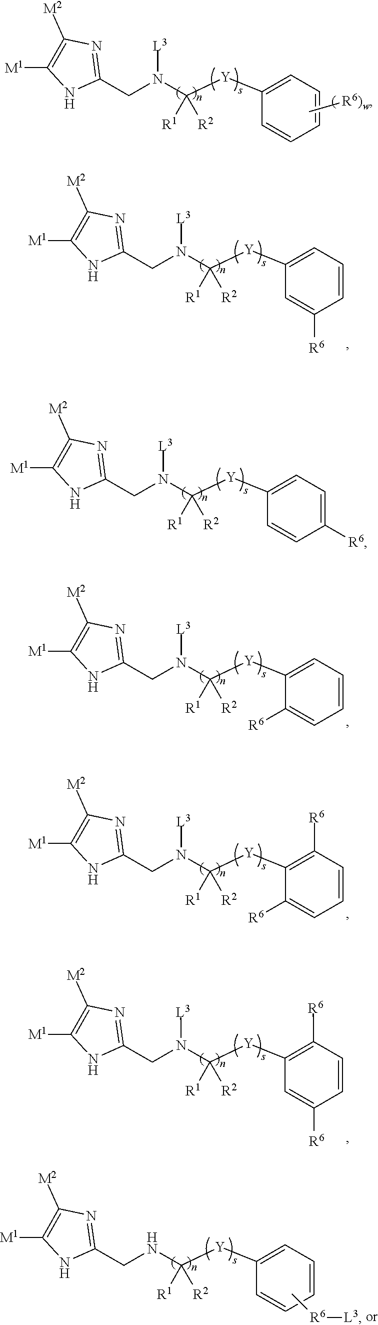

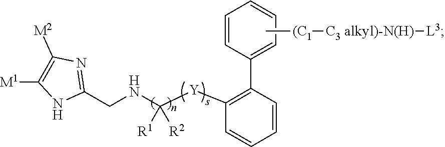

or a salt thereof.

In some aspects, the conjugate comprises a structure as shown in Table 2:

TABLE 2

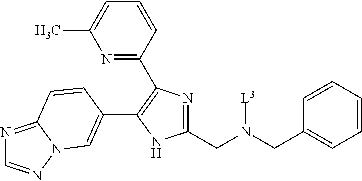

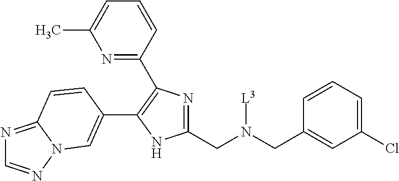

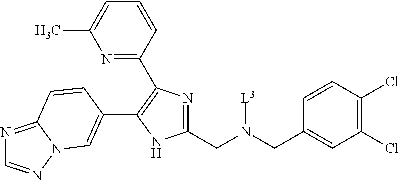

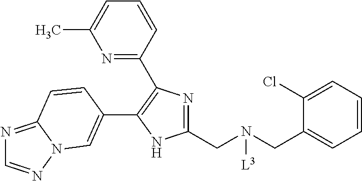

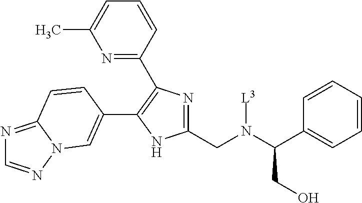

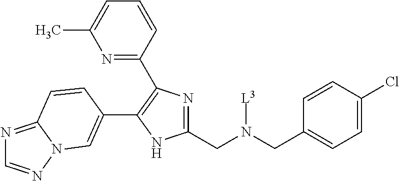

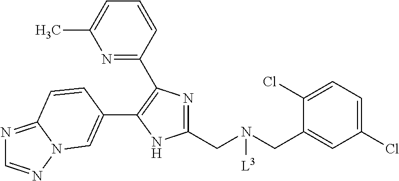

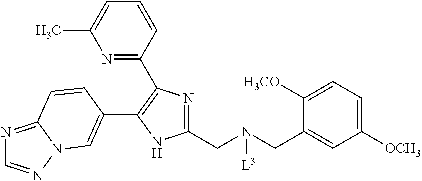

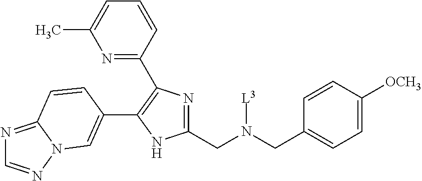

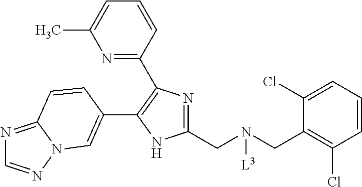

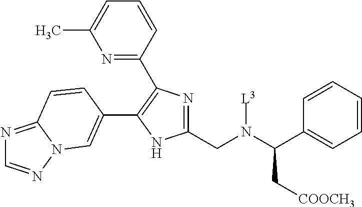

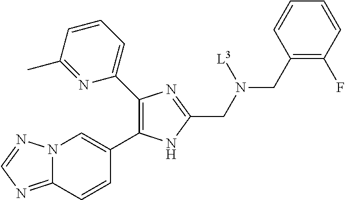

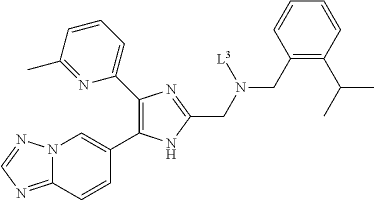

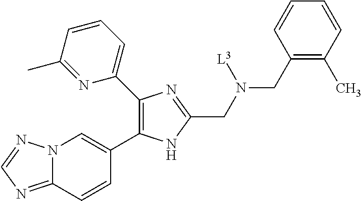

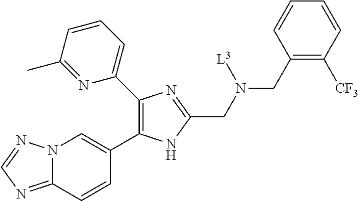

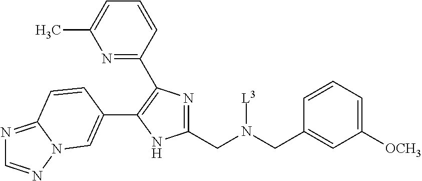

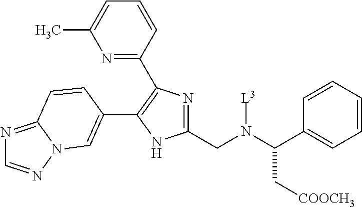

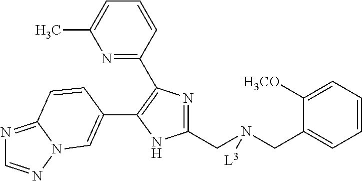

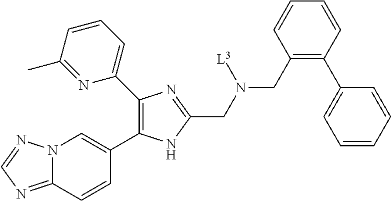

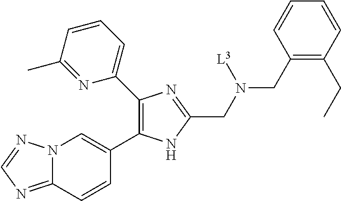

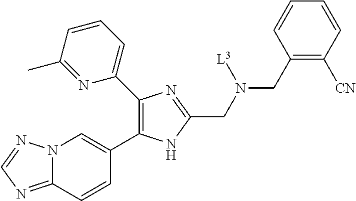

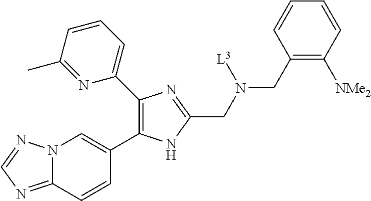

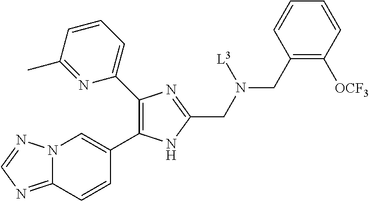

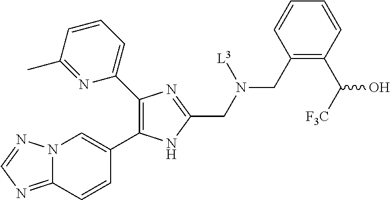

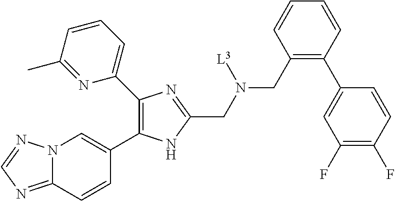

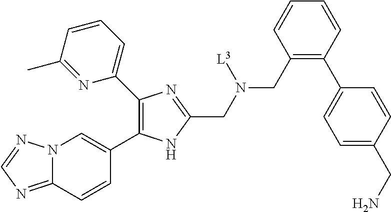

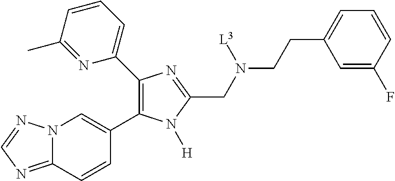

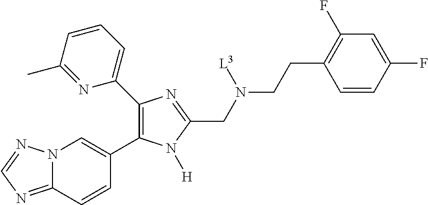

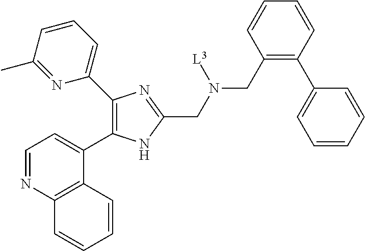

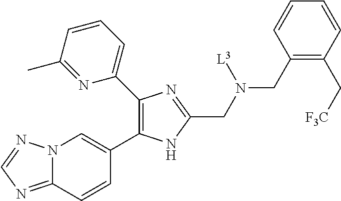

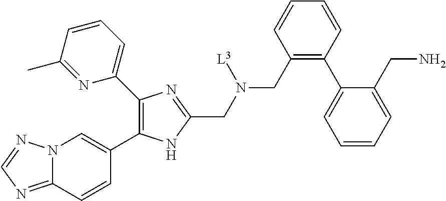

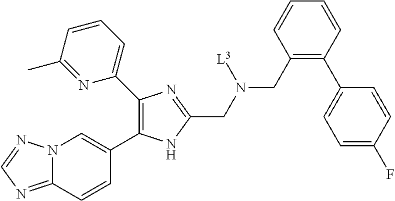

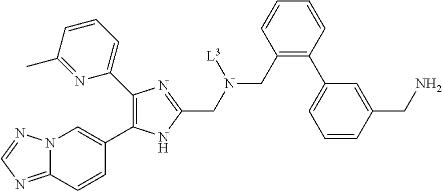

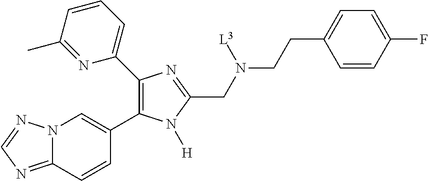

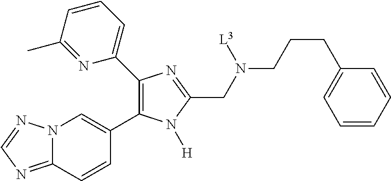

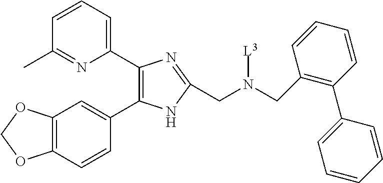

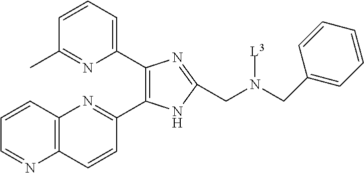

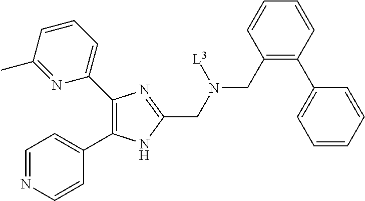

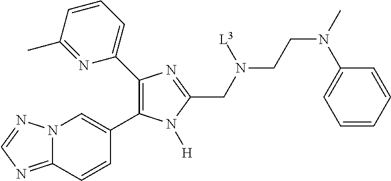

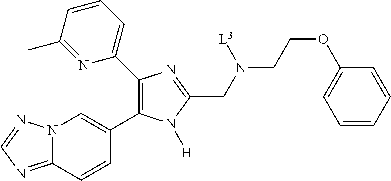

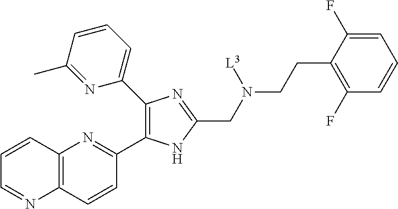

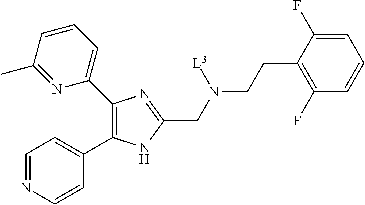

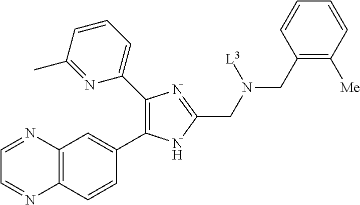

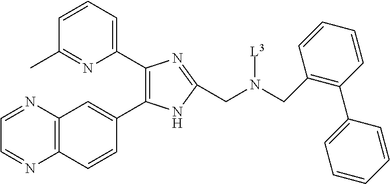

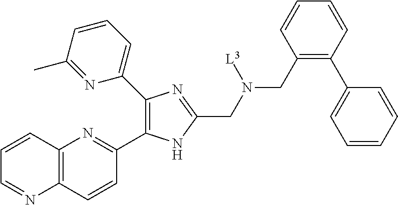

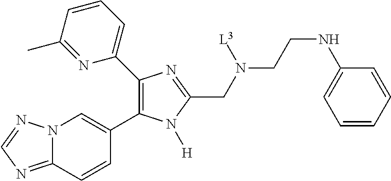

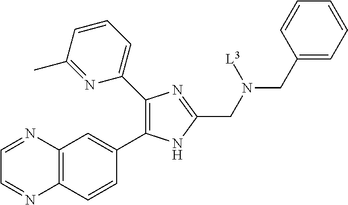

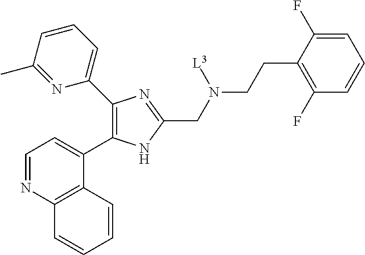

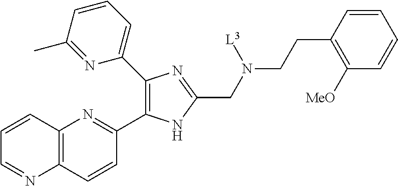

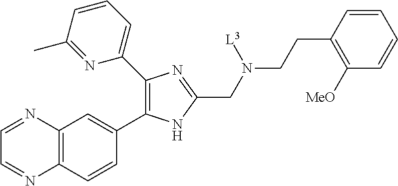

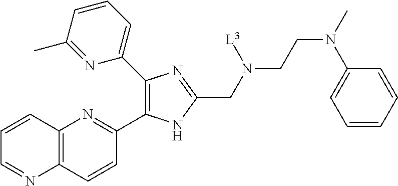

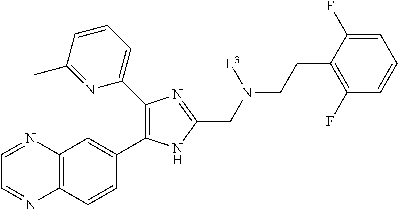

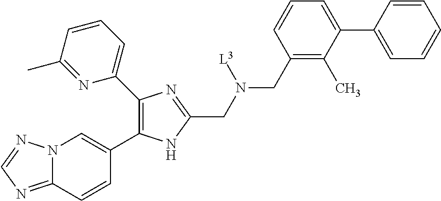

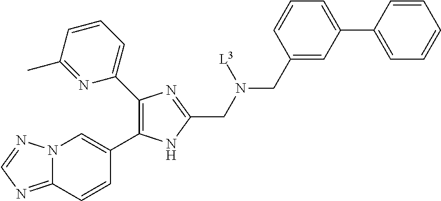

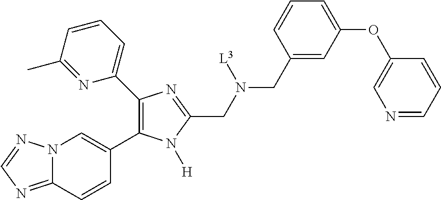

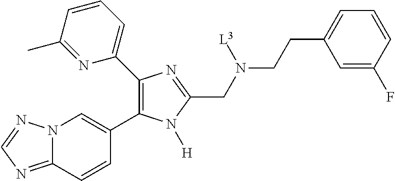

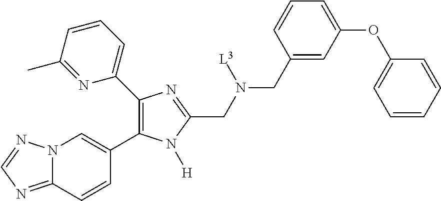

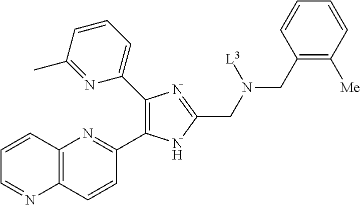

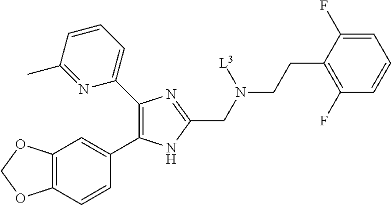

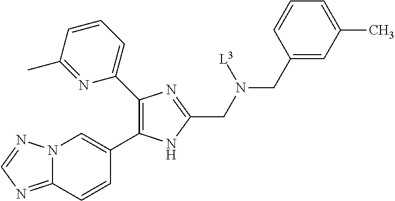

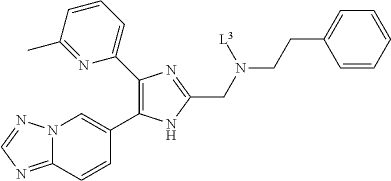

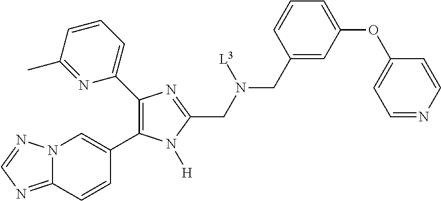

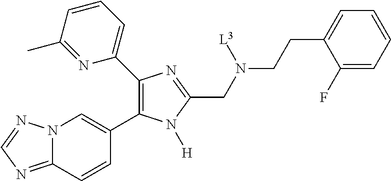

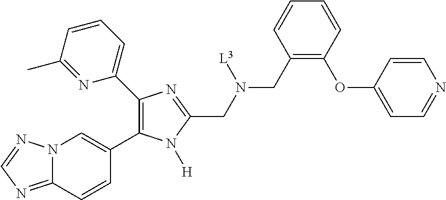

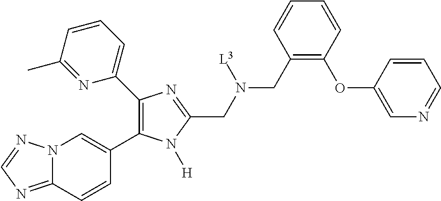

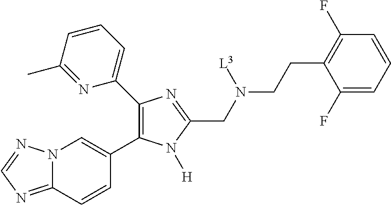

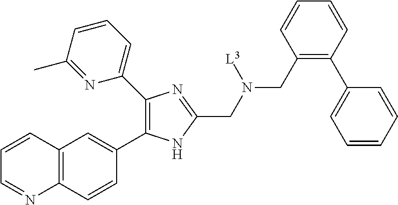

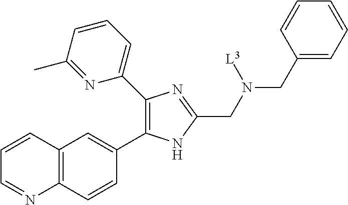

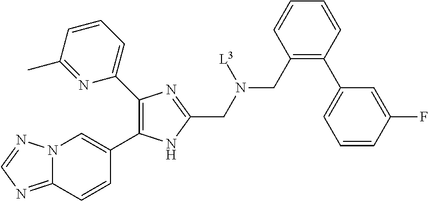

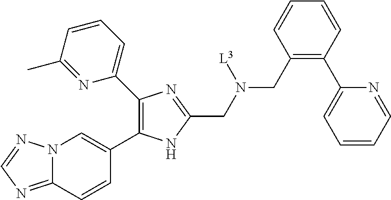

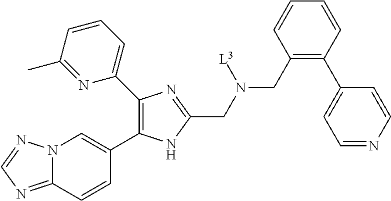

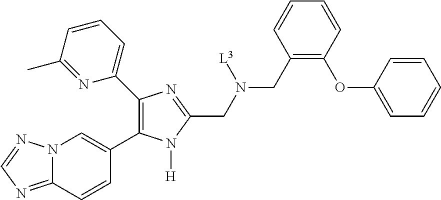

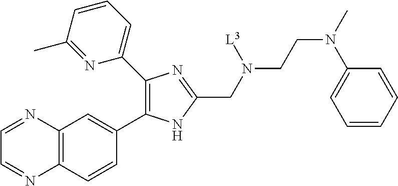

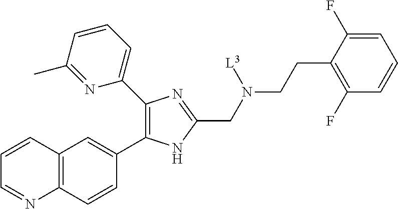

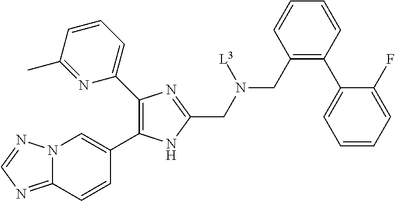

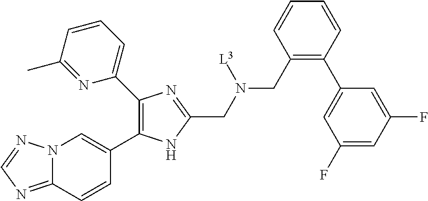

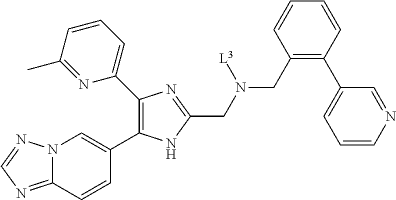

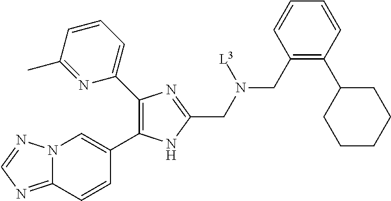

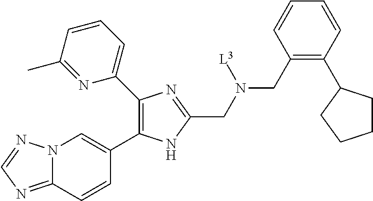

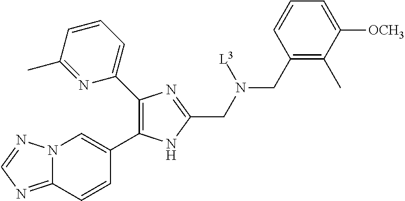

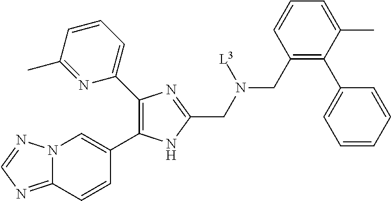

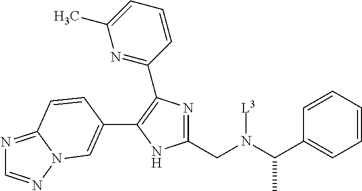

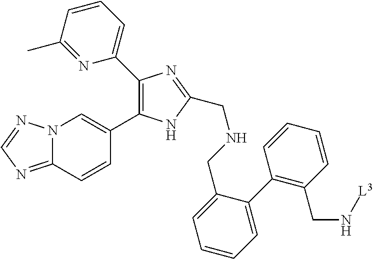

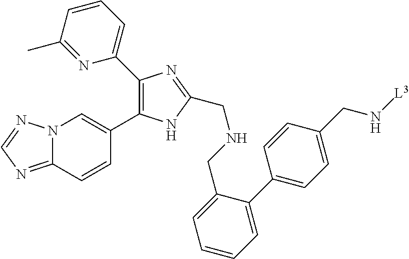

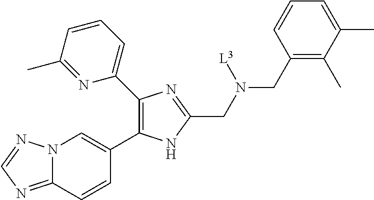

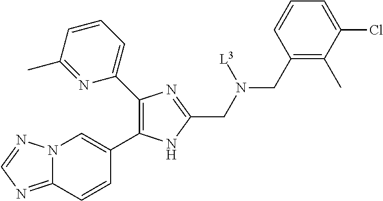

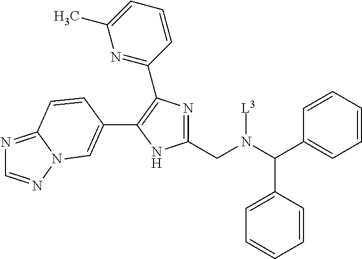

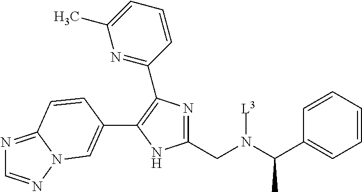

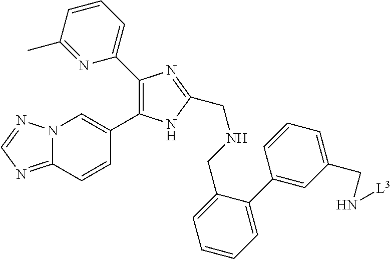

In some aspects, the conjugate comprises one of the following structures, where the maleimide in each structure is replaced with a succinimide moiety

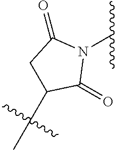 [SMILES: *C1CC(=O)N(*)C1=O]

in the conjugate, which is the point of attachment to the antibody construct:

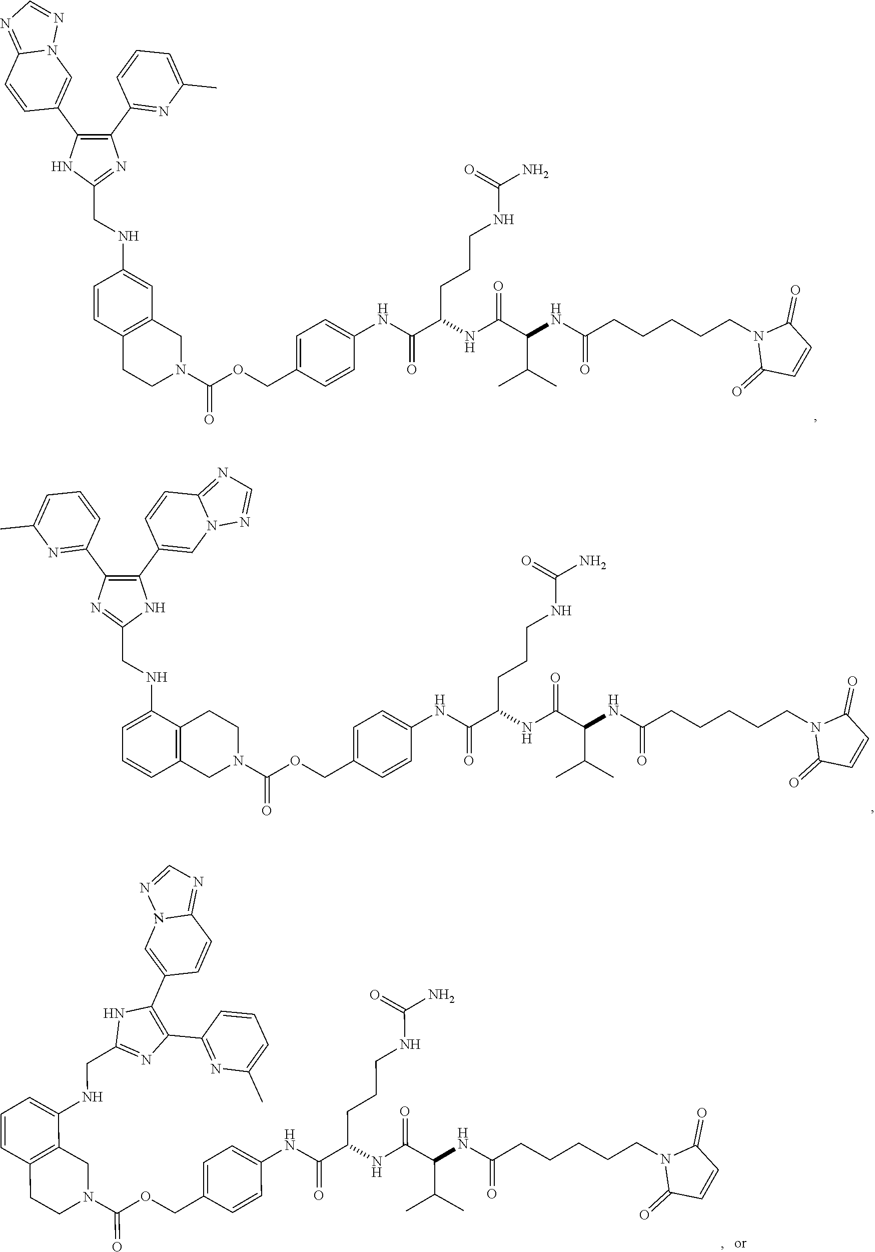

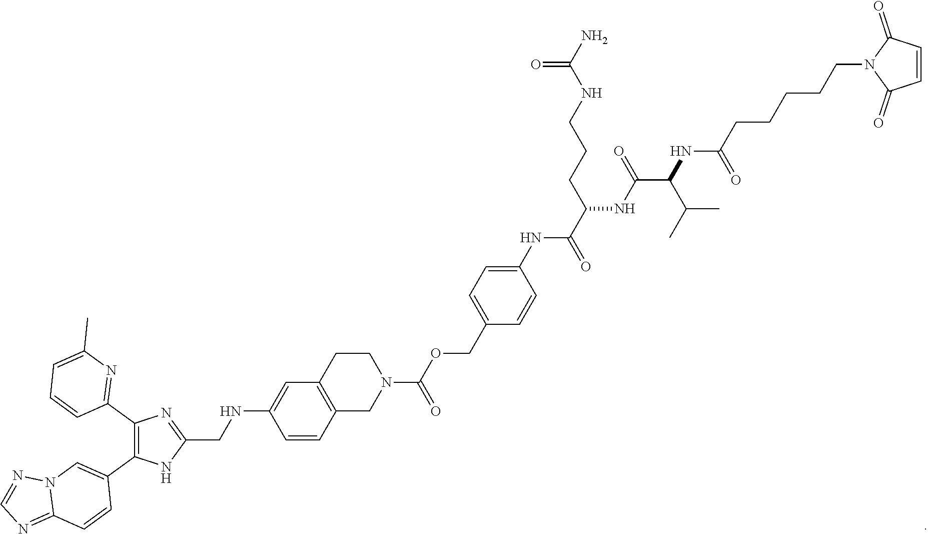 [SMILES: Cc1cccc(-c2nc(CNc3ccc4c(c3)CCN(C(=O)OCc3ccc(NC(=O)[C@H](CCCNC(N)=O)NC(=O)[C@@H](NC(=O)CCCCCN5C(=O)C=CC5=O)C(C)C)cc3)C4)[nH]c2-c2ccc3ncnn3c2)n1]

A TGFβR1 inhibitor-linker compound can be synthesized by various methods known in the art. See, for example, WO2018170179.

As is known by skilled artisans, the linker selected for a particular conjugate may be influenced by a variety of factors, including but not limited to, the site of attachment to the antibody construct, structural constraints of the drug pharmacophore, and the lipophilicity of the drug. The specific linker selected for a conjugate should seek to balance these different factors for the specific conjugate.

### Exemplary Syntheses of TGFβR1 Inhibitor-Linkers

A TGFβR1 inhibitor-linker compound can be synthesized by various methods before being attached to an antibody construct to form the conjugates as described herein. For example, a can be synthesized as shown in Scheme B1.

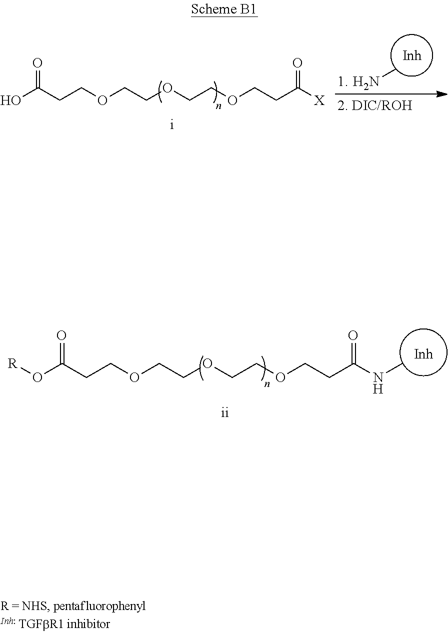

A PEGylated carboxylic acid (i) that has been activated for amide bond formation can be reacted with an appropriately substituted amine containing TGFβR1 inhibitor to afford an intermediate amide. Formation of an activated ester (ii) can be achieved by reaction the intermediate amide-containing carboxylic using a reagent such as N-hydroxysuccinimide or pentafluorophenol in the presence of a coupling agent such as diisopropylcarbodiimide (DIC) to provide compounds (ii).

As another example, TGFβR1 inhibitor-linkers can be synthesized as shown in Scheme B2.

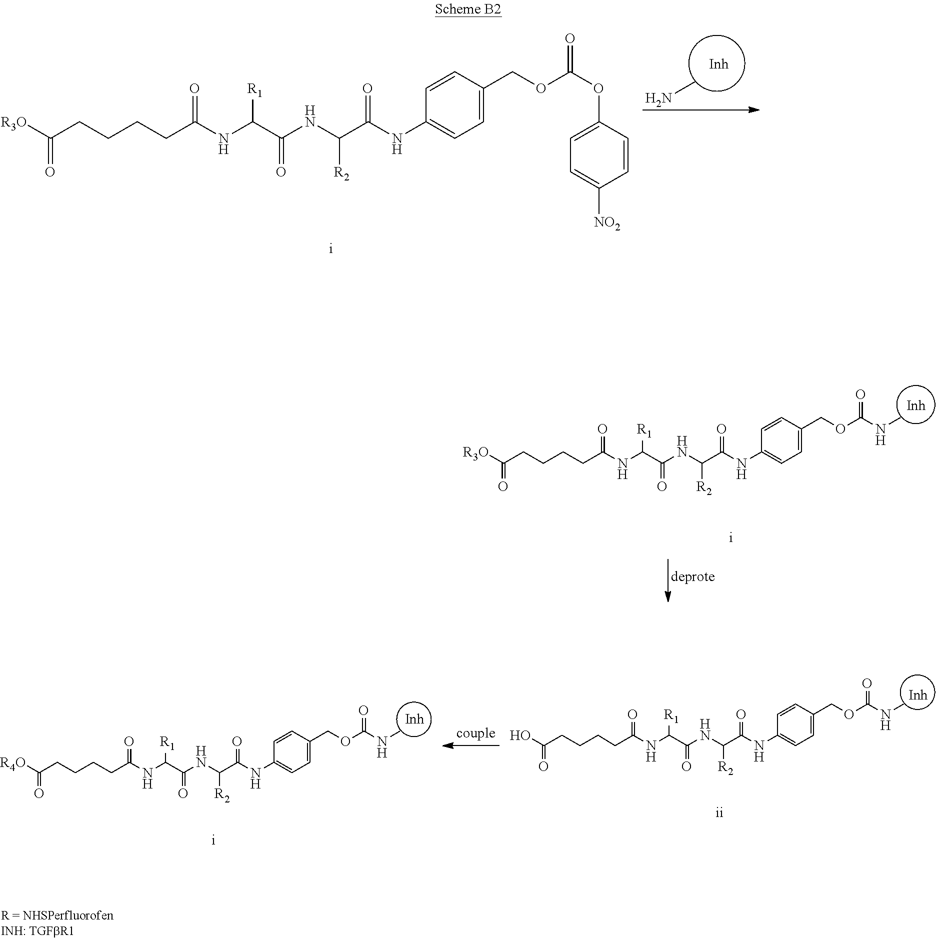

An activated carbonate such as (i) can be reacted with an appropriately substituted amine containing a TGFβR1 inhibitor to afford carbamates (ii) which can be deprotected using standard methods based on the nature of the R₃ ester group. The resulting carboxylic acid (iii) can then by coupled with an activating agent such as N-hydroxysuccinimide or pentafluorophenol to provide compounds (iv).

As an additional example, TGFβR1 inhibitor-linker can be synthesized as shown in Scheme B3.

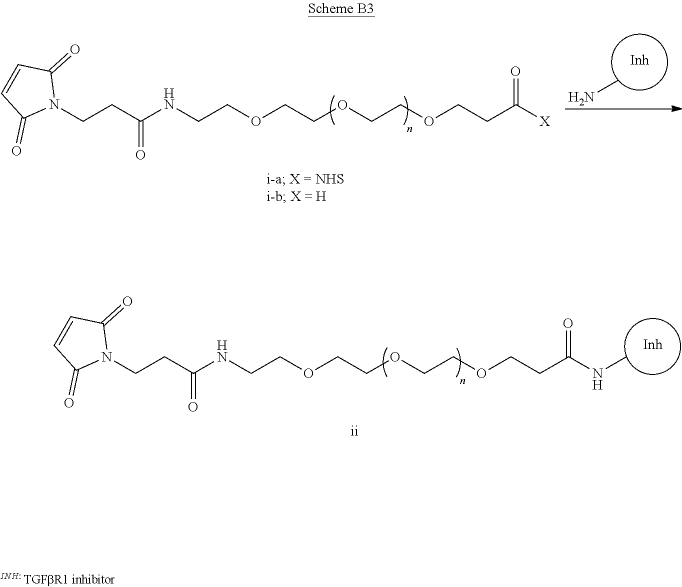

An activated carboxylic ester such as (i-a) can be reacted with an appropriately substituted amine containing TGFβR1 inhibitor to afford amides (ii). Alternatively, carboxylic acids of type (i-b) can be coupled to an appropriately substituted amine containing TGFβR1 inhibitor in the presence of an amide bond forming agent such as dicyclohexycarbodiimde (DCC) to provide the desired TGFβR1 inhibitor-linker.

As an additional example, a TGFβR1 inhibitor-linker can be synthesized as shown in Scheme B4.

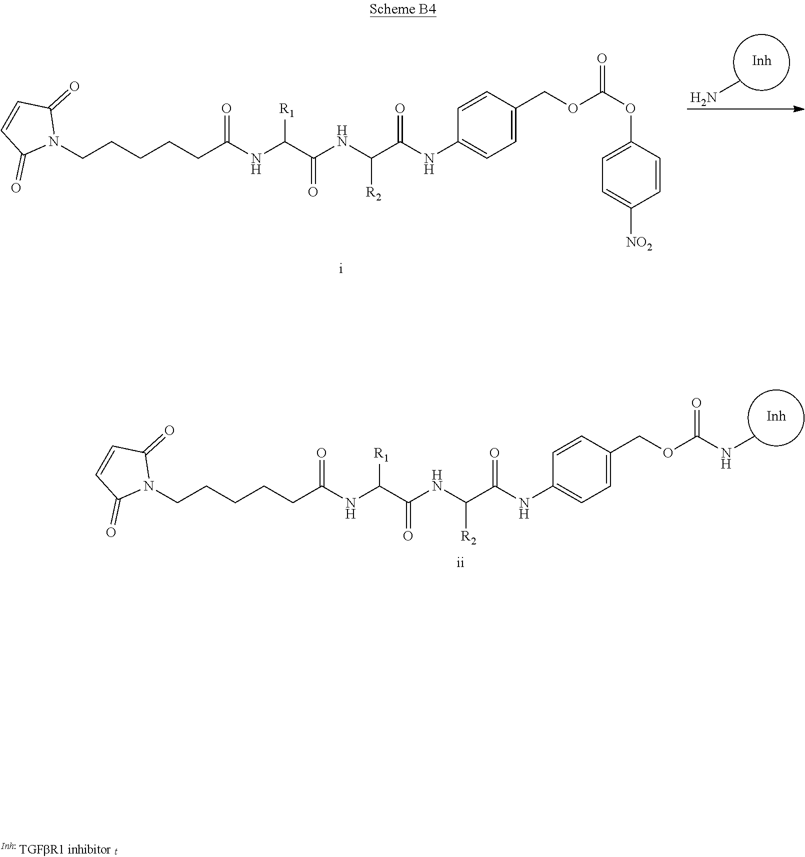

An activated carbonate such as (i) can be reacted with an appropriately substituted amine containing TGFβR1 inhibitor to afford carbamates (ii) as the target TGFβR1 inhibitor.

As an additional example, a TGFβR1 inhibitor-linker can be synthesized as shown in Scheme B5.

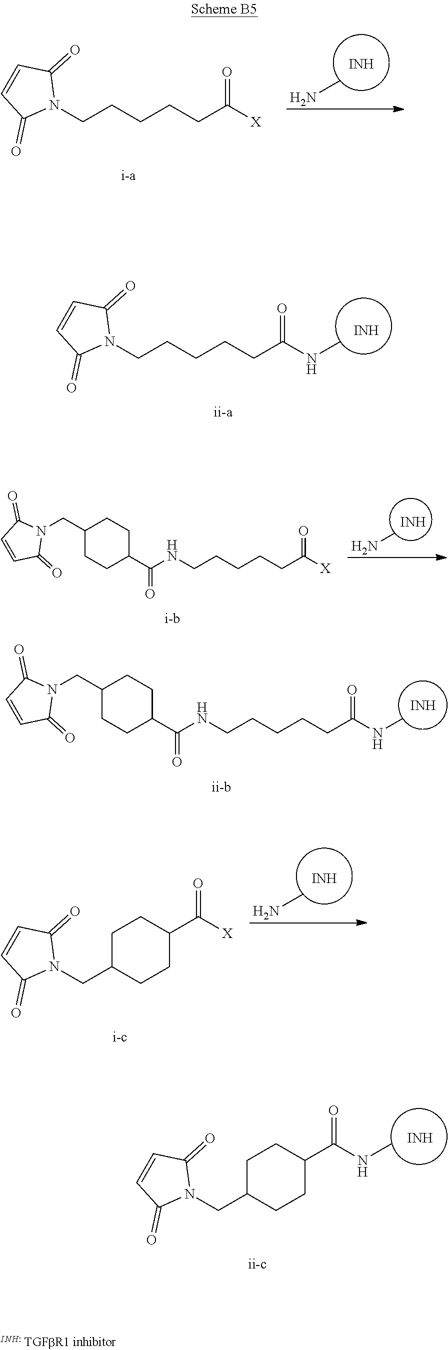

An activated carboxylic acid such as (i-a, i-b, i-c) can be reacted with an appropriately substituted amine containing TGFβR1 inhibitor to afford amides (ii-a, ii-b, ii-c) as the target TGFβR1 inhibitors.

These TGFβR1 inhibitor-linkers can be made by various methods. It is understood that one skilled in the art may be able to make these compounds by similar methods or by combining other methods known to one skilled in the art. It is also understood that one skilled in the art would be able to make, in a similar manner as described herein by using the appropriate starting materials and modifying the synthetic route as needed. Starting materials and reagents can be obtained from commercial vendors or synthesized according to sources known to those skilled in the art or prepared as described herein.

### Pharmaceutical Formulations

The conjugates described herein are useful as pharmaceutical compositions for administration to a subject in need thereof. Pharmaceutical compositions can comprise the conjugates described herein and one or more pharmaceutically acceptable carriers, diluents, excipients, stabilizers, dispersing agents, suspending agents, and/or thickening agents. A pharmaceutical composition can comprise any conjugate described herein. A pharmaceutical composition can further comprise buffers, antibiotics, steroids, carbohydrates, drugs (e.g., chemotherapy drugs), radiation, polypeptides, chelators, adjuvants and/or preservatives.

In a pharmaceutical composition, the conjugates can have an average drug loading. The drug loading, p, is the average number of TGFβR1 inhibitor-linker molecules per antibody construct, or the number of TGFβR1 inhibitors per antibody construct. The variable z can range ranges from 1 to 20, or 1-100. In some conjugates, z is preferably from 1 to 8. The average number of TGFβR1 inhibitors per antibody construct in a preparation may be characterized by conventional means such as mass spectroscopy, HIC, ELISA assay, and HPLC.

Pharmaceutical compositions can be formulated using one or more physiologically-acceptable carriers comprising excipients and auxiliaries. Formulation can be modified depending upon the route of administration chosen. Pharmaceutical compositions comprising a conjugate as described herein can be manufactured, for example, by lyophilizing the conjugate, mixing, dissolving, emulsifying, encapsulating or entrapping the conjugate. The pharmaceutical compositions can also include the conjugates described herein in a free-base form or pharmaceutically-acceptable salt form.

Methods for formulation of the pharmaceutical compositions can include formulating any of the conjugates described herein with one or more inert, pharmaceutically-acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compositions described herein can be lyophilized or in powder form for re-constitution with a suitable vehicle, e.g., sterile pyrogen-free water, before use.

Pharmaceutical compositions of the conjugates described herein can comprise at least a conjugate as an active ingredient, respectively. The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

Pharmaceutical compositions as described herein often further can comprise or be used with one or more additional active compounds as necessary for the particular indication being treated. The active compounds for use in a combination therapy can have complementary activities that do not adversely affect each other. For example, the pharmaceutical composition can be used with or also comprise a cytotoxic agent, cytokine, growth-inhibitory agent, anti-hormonal agent, anti-angiogenic agent, and/or cardioprotectant. Such molecules can be used with or present in combination in amounts that are effective for the purpose intended. In some embodiments, a combination therapy comprises nintedanib or pirfenidone.

The pharmaceutical compositions and formulations can be sterilized. Sterilization can be accomplished by filtration through sterile filtration.

The pharmaceutical compositions described herein can be formulated for administration as an injection. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Injections can be formulated for bolus injection or continuous infusion. Alternatively, the pharmaceutical compositions described herein can be lyophilized or in powder form for reconstitution with a suitable vehicle, e.g., sterile pyrogen-free water, before use.

For parenteral administration, the conjugates can be formulated in a unit dosage injectable form (e.g., use letter solution, suspension, emulsion) in association with a pharmaceutically acceptable parenteral vehicle. Such vehicles can be inherently nontoxic, and non-therapeutic. A vehicle can be water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

Sustained-release preparations can also be prepared. Examples of sustained-release preparations can include semipermeable matrices of solid hydrophobic polymers that can contain the antibody, and these matrices can be in the form of shaped articles (e.g., films or microcapsules). Examples of sustained-release matrices can include polyesters, hydrogels (e.g., poly(2-hydroxyethyl-methacrylate), or poly(vinylalcohol)), polylactides, copolymers of L-glutamic acid and y ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations of the compositions described herein can be prepared for storage by mixing a conjugate with a pharmaceutically acceptable carrier, excipient, and/or a stabilizer. This formulation can be a lyophilized formulation or an aqueous solution. Acceptable carriers, excipients, and/or stabilizers can be nontoxic to recipients at the dosages and concentrations used. Acceptable carriers, excipients, and/or stabilizers can include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

In certain embodiments, the disclosure provides a pharmaceutical composition, comprising a conjugate of a compound of Formula (A-I) or Formula (B-I), or any subformulae or species thereof, and a pharmaceutically acceptable excipient. In certain embodiments, the disclosure provides a pharmaceutical composition, comprising a conjugate of a compound of Formula (A-I) or Formula (B-I), or any subformulae or species thereof, and a pharmaceutically acceptable excipient. In certain embodiments, the average Drug-to-Antibody Ratio (DAR) of the pharmaceutical composition is selected from 1 to 8.

### Therapeutic Applications

The conjugates, pharmaceutical compositions, and methods of the present disclosure can be useful for treating a plurality of different subjects including, but are not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof. In preferred embodiments, conjugates, pharmaceutical compositions, and methods of the present disclosure are used for treating a human.

The conjugates, pharmaceutical compositions, and methods described herein can be useful as a therapeutic, for example a treatment that can be administered to a subject in need thereof. A therapeutic effect can be obtained in a subject by reduction, suppression, remission, or eradication of a disease state, including, but not limited to, a symptom thereof. A therapeutic effect in a subject having a disease or condition, or pre-disposed to have or is beginning to have the disease or condition, can be obtained by a reduction, a suppression, a prevention, a remission, or an eradication of the condition or disease, or pre-condition or pre-disease state.

In practicing the methods described herein, therapeutically-effective amounts of the conjugates, or pharmaceutical compositions described herein can be administered to a subject in need thereof, often for treating and/or preventing a condition or progression thereof. A pharmaceutical composition can affect the physiology of the subject, such as the immune system, inflammatory response, or other physiologic affect. A therapeutically-effective amount can vary widely depending on the severity of the disease, the age and relative health of the subject, the potency of the compounds used, and other factors.

Treat and/or treating can refer to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treat can be used herein to refer to a method that results in some level of treatment or amelioration of the disease or condition, and can contemplate a range of results directed to that end, including but not restricted to prevention of the condition entirely.

Prevent, preventing and the like can refer to the prevention of the disease or condition, e.g., viral infection, in the patient. For example, if an individual at risk of contracting a viral infection is treated with the methods of the present disclosure and does not later become infected with the virus, then the disease has been prevented, at least over a period of time, in that individual.

A therapeutically effective amount can be the amount of conjugates or pharmaceutical compositions or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment, and can be ascertainable by one skilled in the art using known techniques.

The conjugates or pharmaceutical compositions described herein that can be used in therapy can be formulated and dosages established in a fashion consistent with good medical practice taking into account the disorder to be treated, the condition of the individual patient, the site of delivery of the conjugate, or pharmaceutical composition, the method of administration and other factors known to practitioners. The conjugates or pharmaceutical compositions can be prepared according to the description of preparation described herein.

One of ordinary skill in the art would understand that the amount, duration and frequency of administration of a pharmaceutical composition or conjugate described herein to a subject in need thereof depends on several factors including, for example but not limited to, the health of the subject, the specific disease or condition of the patient, the grade or level of a specific disease or condition of the patient, the additional therapeutics the subject is being or has been administered, and the like.

The methods, conjugates and pharmaceutical compositions described herein can be for administration to a subject in need thereof. Often, administration of the conjugates, or pharmaceutical compositions can include routes of administration, non-limiting examples of administration routes include intravenous, intraarterial, subcutaneous, subdural, intramuscular, intracranial, intrasternal, intratumoral, or intraperitoneally. Additionally, a pharmaceutical composition, or conjugate can be administered to a subject by additional routes of administration, for example, by inhalation, oral, dermal, intranasal, or intrathecal administration.

Pharmaceutical compositions or conjugates of the present disclosure can be administered to a subject in need thereof in a first administration, and in one or more additional administrations. The one or more additional administrations can be administered to the subject in need thereof minutes, hours, days, weeks or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week or more than once per month. The conjugates or pharmaceutical compositions can be administered to the subject in need thereof in cycles of 21 days, 14 days, 10 days, 7 days, 4 days or daily over a period of one to seven days.

In one aspect, the disclosure relates to a method for treating a disease mediated by TGFβR1 activity, comprising administering an effective amount of a conjugate or a pharmaceutical composition as described herein to a subject in need thereof. In some aspects, the disease is cancer. In certain embodiments, the cancer is hepatocellular carcinoma (HCC). In further embodiments, the cancer is metastatic liver cancer from colon, lung, breast, neuroendocrine, stomach or pancreatic cancer.

In some aspects, the fibrosis is liver fibrosis. In some aspects, the fibrosis is cancer-associated. In some aspects, the fibrosis is not cancer-associated. In some aspects, the fibrosis is associated with scleroderma, systemic fibrosis, steatohepatitis, non-alcoholic steatohepatitis (NASH), chronic liver viral disease (such as HBV and HCV infection), autoimmune hepatitis, or primary biliary cholangitis.

In one aspect is a method of treating a subject having liver cancer, such as hepatocellular carcinoma (HCC), comprising administering to the subject an effective amount of a conjugate as described herein or a pharmaceutical composition comprising the conjugate. In certain embodiments, the presently described conjugates or pharmaceutical compositions can be used to decrease HCC tumor cell growth and invasiveness. In further embodiments, the presently described conjugates or pharmaceutical compositions can be utilized to enhance liver-localized immune responses against liver cancer, such as HCC.

In some embodiments, the administering is in a regimen that comprises administering the conjugates or pharmaceutical compositions intravenously or subcutaneously. In certain embodiments, the dose level and schedule are chosen to maintain a high ratio of liver exposure to systemic exposure. In further embodiments, the dose and schedule are chosen to attain about an IC₉₀, IC₈₀, IC₅₀ or IC₃₀ of phospho-SMAD2/3 levels in hepatocytes as compared to non-conjugated treatment for at least 24 hours. In still further embodiments, the dose and schedule are chosen to attain about an IC₉₀, IC₈₀, IC₅₀ or IC₃₀ of non-hepatocyte liver cells compared to non-conjugate treatment for 24 hours.

### Increased Dosages and Reduced Side-Effects

In certain embodiments, using a conjugate of this disclosure can allow administration of the conjugate at greater levels of the inhibitor in the form of the conjugate than the level of inhibitor alone. For example, the conjugate can be administered at a level higher than the maximum tolerated dose for that inhibitor administered in the absence of the being conjugated to the antibody construct in the conjugate. In certain embodiments, administration of the conjugate can be associated with fewer side effects than when administered as the inhibitor alone.

### Diseases, Conditions and the Like

The conjugates, pharmaceutical compositions, and methods provided herein can be useful for the treatment of a plurality of diseases, conditions, preventing a disease or a condition in a subject or other therapeutic applications for subjects in need thereof. The conjugates, antibody constructs, pharmaceutical compositions, and methods provided herein can be useful for treatment of liver diseases, such as liver fibrosis or liver cancer. In some aspects, the fibrosis is cancer-associated. In some aspects, the fibrosis is not cancer-associated. In some aspects, the fibrosis is associated with scleroderma, systemic fibrosis, steatohepatitis, non-alcoholic steatohepatitis (NASH), chronic liver viral disease (such as HBV and HCV infection), autoimmune hepatitis, or primary biliary cholangitis.

The disclosure further provides any conjugates disclosed herein for use in a method of treatment of the human or animal body by therapy. Therapy may be by any mechanism disclosed herein, such as inhibiting, reducing, or reducing progression of fibrosis or other liver damage. The disclosure further provides any conjugate disclosed herein for prevention or treatment of any condition disclosed herein, for example, fibrosis or cancer. The disclosure also provides any conjugate or pharmaceutical composition thereof disclosed herein for obtaining any clinical outcome disclosed herein for any condition disclosed herein, such as treating fibrosis or cancer. The disclosure also provides use of any conjugate or pharmaceutical composition thereof disclosed herein in the manufacture of a medicament for preventing or treating any condition disclosed herein.

### EXAMPLES

### General Synthetic Schemes and Examples

The following examples illustrate the various methods of making compounds described herein. The examples are included to further describe some embodiments of the present disclosure and should not be used to limit the scope of the disclosure. The following synthetic schemes are provided for purposes of illustration, not limitation. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

General Scheme 1 for the Preparation of Exemplary ALK5 Inhibitors of Formula (A-I) or (B-I)

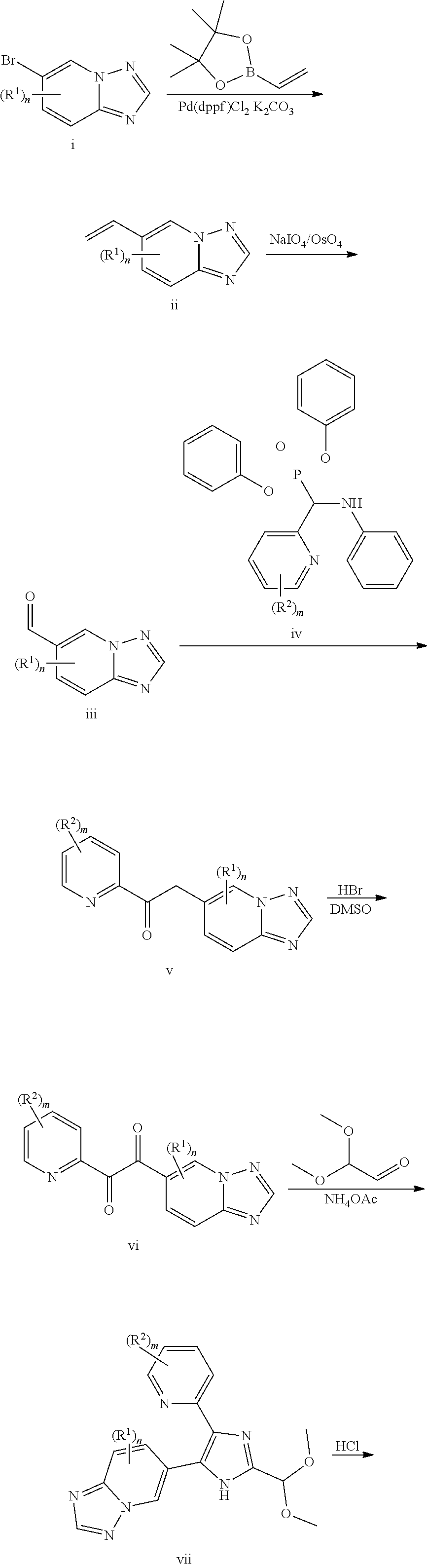

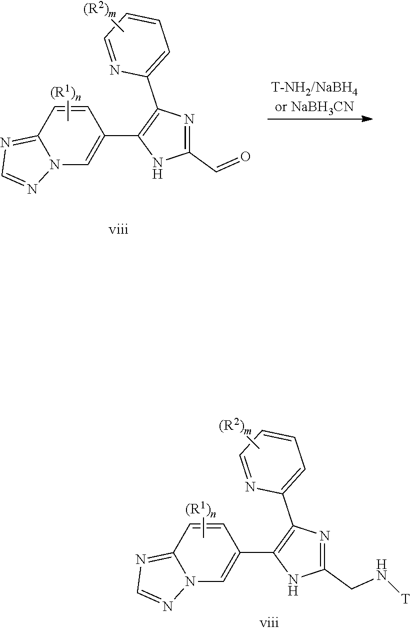

In one method, compounds of Formula (A-I), or compounds of Formula (B-I) wherein Q is CH₂N, are prepared according to Scheme 1. Specifically, R²-substituted pyridine-2-carbaldehydes are reacted with aniline and diphenyl phosphite to give N,P-acetal (iv), which can be further coupled with R¹ substituted [1,2,4]triazolo[1,5-a]pyridine-6-carbaldehydes (iii) followed by hydrolysis in acidic condition to produce a monoketone (v). The monoketone (v) may be oxidized to a diketone (vi) with HBr in DMSO. Certain diketone analogs (vi) were commercially available (e.g., R¹=H and R²=CH³: CAS No. 356560-84-4) and were used as a starting point when appropriate. Diketones (vi) can be condensed with 2,2-dimethoxyacetaldehyde in the presence of ammonium acetate to yield an acetal-protected imidazole (vii), which can be hydrolyzed in acidic condition to produce an imidazole-2-carbaldehyde (viii). The imidazole-2-carbaldehyde (viii) can be reductively aminated in the presence of an amine and a reducing agent such as sodium borohydride or sodium cyanoborohydride to yield a compound of Formula (A-I) or Formula (B-I), where substituents are selected as appropriate.

### Example 1

### Synthesis of 1-(5-([1,2,4]triazolo[1,5-a]pyridin-6-yl)-4-(6-methylpyridin-2-yl)-1H-imidazol-2-yl)-N-benzylmethanamine (Compound 1)

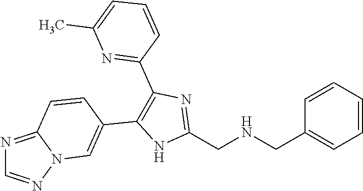 [SMILES: Cc1cccc(-c2nc(CNCc3ccccc3)[nH]c2-c2ccc3ncnn3c2)n1]

Step A. Preparation of Int 1a

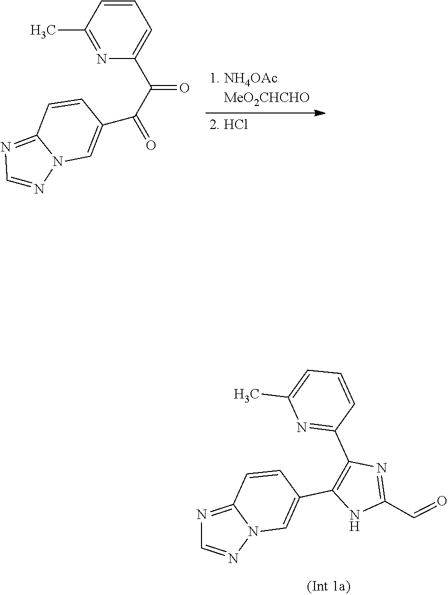 [SMILES: Cc1cccc(C(=O)C(=O)c2ccc3ncnn3c2)n1 ; Cc1cccc(-c2nc(C=O)[nH]c2-c2ccc3ncnn3c2)n1]

To a stirred solution of 1-([1,2,4]triazolo[1,5-a]pyridin-6-yl)-2-(6-methylpyridin-2-yl)ethane-1,2-dione (CAS 356560-84-4; 2.7 g, 10.1 mmol) in a 2:1 mixture of tert-butyl methyl ether and methanol (25 mL) were added 60% 2,2-dimethoxyacetaldehyde in H₂O (3.5 mL, 20 mmol) and NH₄OAc (1.95 g, 25.2 mmol). The mixture was stirred at room temperature for 5 h before the solvent was removed. The pH of the reaction mixture was adjusted to 8 with saturated aqueous NaHCO₃ solution and extracted with CH₂C₂ (2×10 mL). The combined organic extracts were washed with brine (10 mL) and dried over anhydrous Na₂SO₄, filtered, and evaporated. The residue was purified on silica gel (ISCO gold, 40 g; 0% to 20% CH₂Cl₂/MeOH over 15 min) to give the desired imidazole product, which was dissolved in 1 N HCl (20 mL) and heated at 70° C. for 4 h. The reaction mixture was allowed to cool to 0° C. and then it was neutralized with saturated aqueous NaHCO₃ solution. The precipitate was collected and washed with water (20 mL) and ether (40 mL) to give Int 1a as a yellow-brown solid. ¹H NMR (DMSO-d⁶) δ 10.0 (dd, J=1.6, 0.8 Hz, 1H), 9.56 (s, 1H), 8.42 (s, 1H), 8.27 (dd, J=9.2, 1.6 Hz, 1H), 7.82 (br d, J=0.8 Hz, 1H), 7.72 (dd, J=9.2, 0.8 Hz, 1H), 7.65 (t, J=7.8 Hz, 1H), 7.05 (d, J=7.6 Hz, 1H), 2.46 (s, 3H). LCMS (M+H)=305.1.

Step B. Preparation of Compound 1

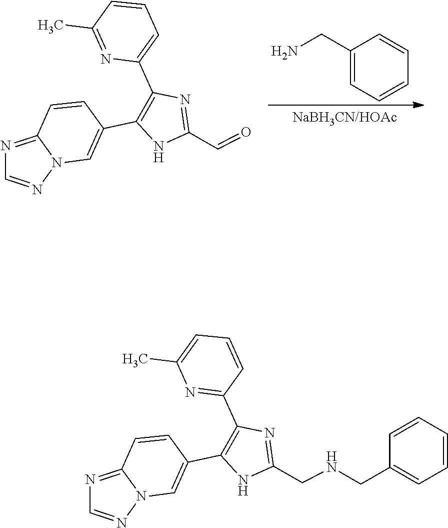 [SMILES: Cc1cccc(-c2nc(C=O)[nH]c2-c2ccc3ncnn3c2)n1 ; NCc1ccccc1 ; Cc1cccc(-c2nc(CNCc3ccccc3)[nH]c2-c2ccc3ncnn3c2)n1]

To a solution of 4-(6-methyl-2-pyridyl)-5-([1,2,4]triazolo[1,5-a]pyridin-6-yl)-1H-imidazole-2-carbaldehyde (200 mg, 0.66 mmol) in dichloroethane (30 mL) was added acetic acid (79 mg, 1.3 mmol, 75 μL) and phenylmethanamine (106 mg, 0.99 mmol). The mixture was stirred at 60° C. for 2 h then cooled to 0° C. Methanol (20 mL) and THE (10 mL) were added followed by NaBH₃CN (165 mg, 2.63 mmol, 4.0 eq) and then the reaction mixture was allowed to warm to 15° C. and stirred for an additional 3 h, at which time LCMS showed the reaction to be complete. The reaction mixture was quenched by addition of water (0.10 mL) at 0° C. and was then concentrated under reduced pressure to give a residue that was purified by silica gel chromatography (0→10% MeOH in DCM) to afford 240 mg of 1-(5-([1,2,4]triazolo[1,5-a]pyridin-6-yl)-4-(6-methylpyridin-2-yl)-1H-imidazol-2-yl)-N-benzylmethanamine as an off-white solid. ¹H NMR (DMSO-d₆, 400 MHz) δ 9.54 (s, 1H), 8.55 (s, 1H), 7.99-7.96 (m, 1H), 7.92-7.88 (m, 1H), 7.79 (t, J=7.8 Hz, 1H), 7.58-7.53 (m, 3H), 7.49-7.41 (m, 3H), 7.26 (d, J=7.7 Hz, 1H), 4.37 (d, J=12.8 Hz, 4H), 2.52 (s, 3H). LCMS M/z 396.1 [M+H]⁺.

### Example 2

### Synthesis of 6-(4-(6-methylpyridin-2-yl)-2-(piperidin-4-yl)-1H-imidazol-5-yl)-[1,2,4]triazolo[1,5-a]pyridine (Compound 1.47)

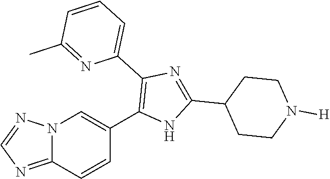 [SMILES: Cc1cccc(-c2nc(C3CCNCC3)[nH]c2-c2ccc3ncnn3c2)n1]

Step A. Preparation of Int. 1.47a

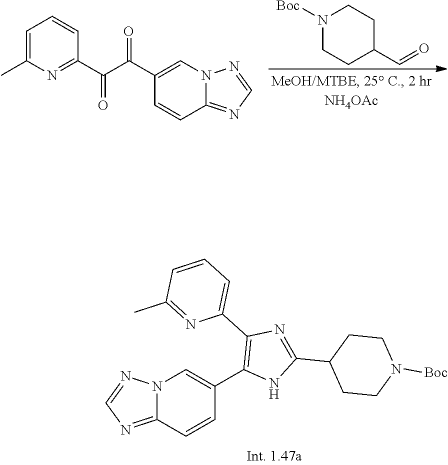 [SMILES: Cc1cccc(C(=O)C(=O)c2ccc3ncnn3c2)n1 ; CC(C)(C)OC(=O)N1CCC(C=O)CC1 ; Cc1cccc(-c2nc(C3CCN(C(=O)OC(C)(C)C)CC3)[nH]c2-c2ccc3ncnn3c2)n1]

To a solution of 1-(6-methyl-2-pyridyl)-2-([1,2,4]triazolo[1,5-a]pyridin-6-yl) ethane-1,2-dione (0.2 g, 751.16 umol, 1 eq) and tert-butyl 4-formylpiperidine-1-carboxylate (160.20 mg, 751.16 umol, 1 eq) in MTBE (5 mL) and MeOH (10 mL) was added NH₄OAc (289.51 mg, 3.76 mmol, 5 eq). The mixture was stirred at 25° C. for 4 h. The reaction mixture was quenched by addition of H₂O (20 mL), and was extracted with EtOAc (20 mL×3). The combined organic layers were washed with brine (50 mL), dried over Na₂SO₄, filtered and concentrated under reduced pressure to give tert-butyl 4-(5-([1,2,4]triazolo[1,5-a]pyridin-6-yl)-4-(6-methylpyridin-2-yl)-1H-imidazol-2-yl)piperidine-1-carboxylate (0.3 g, crude) was obtained as a yellow solid.

Step B. Preparation of Compound 1.47

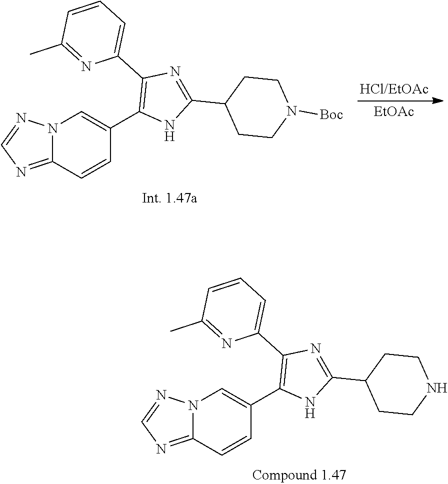 [SMILES: "EtOAc" ; Cc1cccc(-c2nc(C3CCN(C(=O)OC(C)(C)C)CC3)[nH]c2-c2ccc3ncnn3c2)n1 ; Cc1cccc(-c2nc(C3CCNCC3)[nH]c2-c2ccc3ncnn3c2)n1]

To a solution of tert-butyl 4-[4-(6-methyl-2-pyridyl)-5-([1,2,4]triazolo[1,5-a]pyridin-6-yl)-1H-imidazol-2-yl]piperidine-1-carboxylate (Int. 1.47a) (0.3 g, 652.82 umol, 1 eq) in DCM (10 mL) was added HCl/EtOAc (4 M, 326.41 uL, 2 eq). The mixture was stirred at 25° C. for 1 h. The reaction mixture was concentrated under reduced pressure to give a residue. The residue was purified by prep-HPLC (column: Nano-micro Kromasil C18 100*30 mm Sum; mobile phase: [water (0.1% TFA)—ACN]; B %: 1%-20%, 10 min) and freeze-drying. Compound 6-[4-(6-methyl-2-pyridyl)-2-(4-piperidyl)-1H-imidazol-5-yl]-[1,2,4]triazolo[1,5-a]pyridine (Compound 1.47) (0.17 g, 359.07 umol, 55.00% yield, TFA) was obtained as a yellow solid. ¹H NMR (CDCl₃, 400 MHz) δ 9.44 (s, 1H), 8.94 (s, 1H), 8.65 (d, J=6.0 Hz, 1H), 8.60 (s, 1H), 7.94 (d, J=9.2 Hz, 1H), 7.87-7.80 (m, 3H), 7.52-7.47 (m, 1H), 7.36 (d, J=7.6 Hz, 1H), 3.86 (s, 1H), 3.46 (d, J=12.4 Hz, 2H), 3.41-3.33, 1H), 3.17-3.04 (m, 2H), 2.56-2.53 (m, 3H), 2.27 (d, J=12.4 Hz, 2H), 2.11-2.00 (m, 2H); HPLC: 96.356 (220 nm), 98.486 (254 nm); MS (ESI): mass calcd. for C₂₀H₂₁N₇ 359.19, m/z found 360.0[M+H]⁺.

The compounds in Tables 3 and 4 were prepared in a manner similar to that described for Compounds 1 or 1.47.

TABLE 3

| Cmpd. | | |
|---|---|---|
| No. | Structure | ¹H NMR |
| 1 | 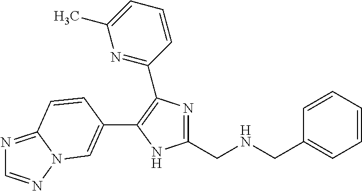 [SMILES: Cc1cccc(-c2nc(CNCc3ccccc3)[nH]c2-c2ccc3ncnn3c2)n1] | ¹H NMR (DMSO-d₆, 400 MHz) δ 9.54 (s, 1H), 8.55 (s, 1H), 7.99-7.96 (m, 1H), 7.92-7.88 (m, 1H), 7.79 (t, J = 7.8 Hz, 1H), 7.58-7.53 (m, 3H), 7.49-7.41 (m, 3H), 7.26 (d, J = 7.7 Hz, 1H), 4.37 (d, J = 12.8 Hz, 4H), 2.52 (s, 3H); m/z 396.1 [M + H]⁺ |
| 2 | 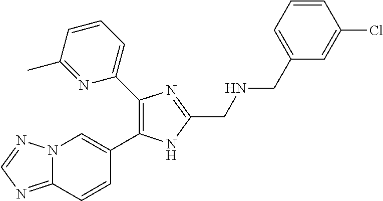 [SMILES: Cc1cccc(-c2nc(CNCc3cccc(Cl)c3)[nH]c2-c2ccc3ncnn3c2)n1] | ¹H NMR (DMSO-d₆, 400 MHz) δ 9.67 (s, 1H), 9.50 (s, 1H), 8.55 (s, 1H), 8.00-7.87 (m, 2H), 7.75 (t, J = 7.6 Hz, 1H), 7.68 (s, 1H), 7.55-7.48 (m, 4H), 7.22 (d, J = 8.0 Hz, 1H), 4.39 (s, 2H), 4.33 (s, 2H), 2.48 (s, 3H); m/z = 430.1 [M + H]⁺. |
| 3 | 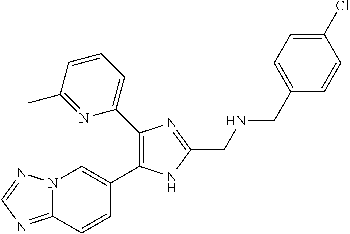 [SMILES: Cc1cccc(-c2nc(CNCc3ccc(Cl)cc3)[nH]c2-c2ccc3ncnn3c2)n1] | ¹H NMR (DMSO-d₆, 400 MHz) δ 9.50 (s, 1H), 8.56 (s, 1H), 7.97-7.87 (m, 1H), 7.79 (t, J = 8.0 Hz, 1H), 7.59- 7.51 (m, 5H), 7.27 (d, J = 7.6 Hz, 1H), 4.38-7.33 (m, 4H), 2.52 (s, 3H); m/z = 430.3 [M + H]⁺ |
| 4 | 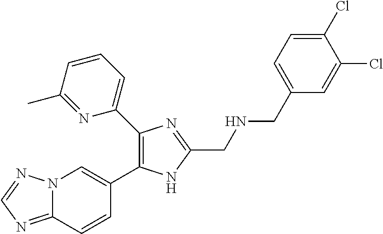 [SMILES: Cc1cccc(-c2nc(CNCc3ccc(Cl)c(Cl)c3)[nH]c2-c2ccc3ncnn3c2)n1] | ¹H NMR (MeOD, 400 MHz) δ 9.23-9.21 (m, 1H), 8.51 (s, 1H), 7.96 (t, J = 7.8 Hz, 1H), 7.88 (t, J = 1.6 Hz, 2H), 7.78 (d, J = 2.2 Hz, 1H), 7.67-7.63 (m, 1H), 7.57 (d, J = 8.0 Hz, 1H), 7.51-7.46 (m, 2H), 4.48 (d, J = 4.6 Hz, 4H), 2.71 (s, 3H); m/z = 464.1 [M + H]⁺ |
| 5 | 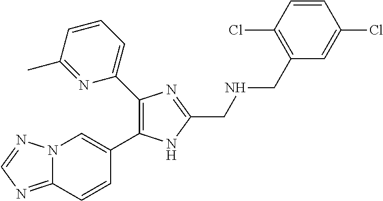 [SMILES: Cc1cccc(-c2nc(CNCc3cc(Cl)ccc3Cl)[nH]c2-c2ccc3ncnn3c2)n1] | ¹H NMR (MeOD, 400 MHz) δ 9.23 (t, J = 1.4 Hz, 1H), 8.52 (s, 1H), 7.97 (t, J = 7.8 Hz, 1H), 7.89 (dd, J = 1.4, 2.2 Hz, 2H), 7.73 (d, J = 2.4 Hz, 1H), 7.60-7.53 (m, 2H), 7.51-7.46 (m, 2H), 4.63 (s, 2H), 4.55 (s, 2H), 2.71 (s, 3H); m/z = 464.1 [M + H]⁺ |
| 6 | 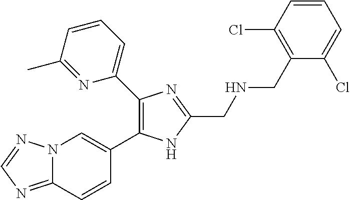 [SMILES: Cc1cccc(-c2nc(CNCc3c(Cl)cccc3Cl)[nH]c2-c2ccc3ncnn3c2)n1] | ¹H NMR (CDCl₃, 400 MHz) δ 8.93 (s, 1H), 8.44 (s, 1H), 7.98-7.86 (m, 2H), 7.70-7.61 (m, 1H), 7.45-7.39 (m, 4H), 7.38-7.33 (m, 1H), 4.73 (s, 2H), 4.55 (s, 2H), 2.89 (s, 3H); m/z = 464.1 [M + H]⁺ |
| 7 | 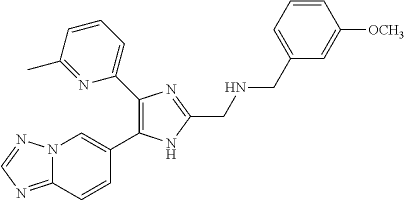 [SMILES: COc1cccc(CNCc2nc(-c3cccc(C)n3)c(-c3ccc4ncnn4c3)[nH]2)c1] | ¹H NMR (MeOD, 400 MHz) δ 9.15 (s, 1H), 8.42 (s, 1H), 7.87 (dd, J = 8.0 Hz, J = 4.0 Hz 1H), 7.75 (d, J = 8.4 Hz, 1H), 7.63 (t, J = 8.0 Hz, 1H), 7.34 (d, J = 7.6 Hz, 1H), 7.23-7.15 (m, 2H), 6.95-6.92 (m, 2H), 6.78 (dd, J = 12.0 Hz, J = 4.0 Hz 1H), 3.94 (s, 2H), 3.82 (s, 2H), 3.77 (s, 3H), 2.53 (s, 3H); m/z = 426.2 [M + H]⁺ |
| 8 | 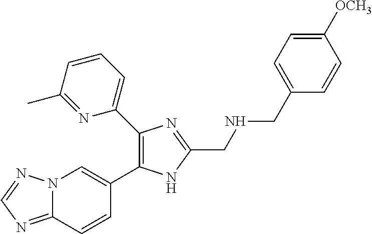 [SMILES: COc1ccc(CNCc2nc(-c3cccc(C)n3)c(-c3ccc4ncnn4c3)[nH]2)cc1] | ¹H NMR (DMSO-d6, 400 MHz) δ 9.49 (s, 1H), 8.56 (s, 1H), 7.97-7.89 (m, 2H), 7.81 (t, J = 7.6 Hz, 1H), 7.53 (d, J = 8.0 Hz, 1H), 7.48 (d, J = 8.8 Hz, 2H), 7.28 (d, J = 7.6 Hz, 1H), 7.02 (d, J = 6.8 Hz 2H), 4.30 (d, J = 2.0 Hz, 4H), 3.77 (s, 3H), 2.53 (s, 3H); m/z = 426.0 [M + H]⁺ |
| 9 | 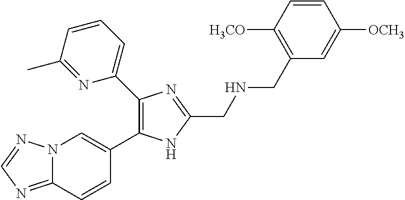 [SMILES: COc1ccc(OC)c(CNCc2nc(-c3cccc(C)n3)c(-c3ccc4ncnn4c3)[nH]2)c1] | ¹H NMR (MeOD, 400 MHz) δ 9.18 (s, 1H), 8.54 (s, 1H), 8.11 (t, J = 8.0 Hz, 1H), 7.95-7.81 (m, 2H), 7.62 (dd, J = 14.6, 8.0 Hz, 2H), 7.02 (s, 3H), 4.45 (d, J = 10.8 Hz, 4H), 3.87 (s, 3H), 3.75 (s, 3H), 2.79 (s, 3H); m/z = 456.1 [M + H]⁺ |
| 10 | 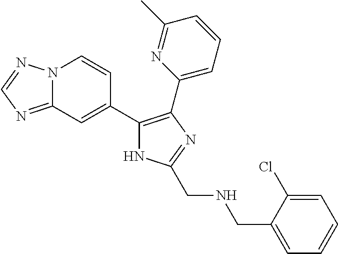 [SMILES: Cc1cccc(-c2nc(CNCc3ccccc3Cl)[nH]c2-c2ccn3ncnc3c2)n1] | ¹H NMR (MeOD, 400 MHz) δ 9.17 (s, 1H), 8.43 (s, 1H), 7.88 (dd, J = 1.6, 9.4 Hz, 1H), 7.78-7.74 (m, 1H), 7.67 (t, J = 7.8 Hz, 1H), 7.50 (dd, J = 1.6, 7.2 Hz, 1H), 7.39-7.35 (m, 2H), 7.31-7.22 (m, 2H), 7.18 (d, J = 7.8 Hz, 1H), 3.98 (d, J = 4.0 Hz, 4H), 2.51 (s, 3H); m/z = 430.2 [M + H]⁺ |
| 11 | 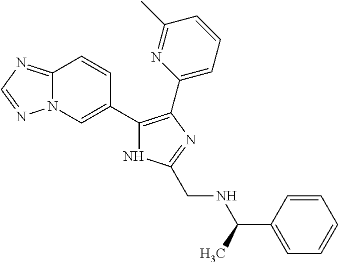 [SMILES: Cc1cccc(-c2nc(CN[C@H](C)c3ccccc3)[nH]c2-c2ccc3ncnn3c2)n1] | ¹H NMR (MeOD, 400 MHz) δ 9.15 (s, 1H), 8.54 (s, 1H), 8.15 (t, J = 8.0 Hz, 1H), 7.95-7.79 (m, 2H), 7.65 (d, J = 7.6 Hz, J = 4.4 Hz 2H), 7.58 (d, J = 6.8 Hz, 2H), 7.53- 7.43 (m, 3H), 4.70 (q, J = 6.4 Hz, 1H), 4.42-4.15 (m, 2H), 2.83 (s, 3H), 1.81 (d, J = 6.8 Hz, 3H); m/z = 410.1 [M + H]⁺ |
| 12 | 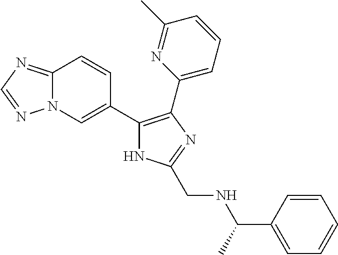 [SMILES: Cc1cccc(-c2nc(CN[C@@H](C)c3ccccc3)[nH]c2-c2ccc3ncnn3c2)n1] | ¹H NMR (MeOD, 400 MHz) δ 9.16 (s, 1H), 8.53 (s, 1H), 8.08 (t, J = 7.8 Hz, 1H), 7.91-7.88 (m, 1H), 7.85-7.81 (m, 1H), 7.58-7.54 (m, 2H), 7.50-7.46 (m, 3H), 4.70 (q, J = 6.8 Hz, 1H), 4.37-4.31 (m, 1H), 4.23-4.18 (m, 1H), 2.78 (s, 3H), 1.80 (d, J = 6.8 Hz, 3H); m/z = 410.1 [M + H]⁺ |
| 13 | 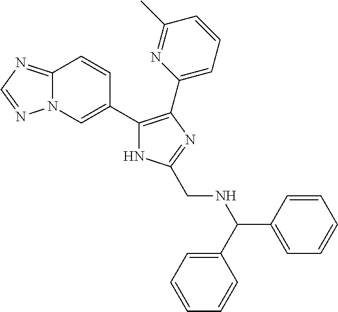 [SMILES: Cc1cccc(-c2nc(CNC(c3ccccc3)c3ccccc3)[nH]c2-c2ccc3ncnn3c2)n1] | ¹H NMR (MeOD, 400 MHz) δ 9.13 (s, 1H), 8.40 (s, 1H), 7.89-7.58 (m, 3H), 7.47-7.09 (m, 12H), 4.93 (s, 1H), 3.91 (s, 2H), 2.49 (s, 3H); m/z = 472.3 [M + H]⁺ |
| 14 | 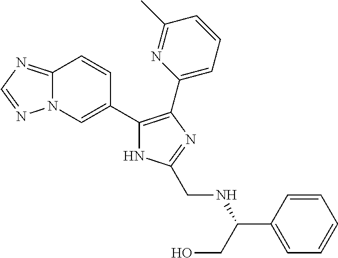 [SMILES: Cc1cccc(-c2nc(CN[C@@H](CO)c3ccccc3)[nH]c2-c2ccc3ncnn3c2)n1] | ¹H NMR (MeOD, 400 MHz) δ 9.15 (s, 1H), 8.42 (s, 1H), 7.91-7.62 (m, 3H), 7.41-7.14 (m, 7H), 3.94-3.77 (m, 3H), 3.71-3.58 (m, 2H), 2.51 (s, 3H) m/z = 426.2 [M + H]⁺ |
| 15 | 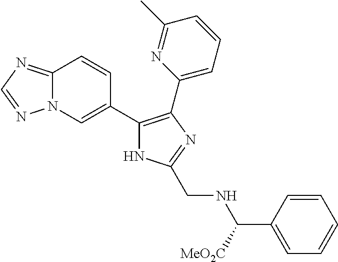 [SMILES: COC(=O)[C@H](NCc1nc(-c2cccc(C)n2)c(-c2ccc3ncnn3c2)[nH]1)c1ccccc1] | ¹H NMR (MeOD, 400 MHz) δ 9.17 (br s, 1H), 8.43 (s, 1H), 7.91-7.62 (m, 3H), 7.51-7.26 (m, 6H), 7.18 (d, J = 7.6 Hz, 1H), 4.63-4.48 (m, 1H), 3.92 (d, J = 2.2 Hz, 2H), 3.67 (s, 3H), 2.51 (s, 3H); m/z = 454.2 [M + H]⁺ |
| 16 | 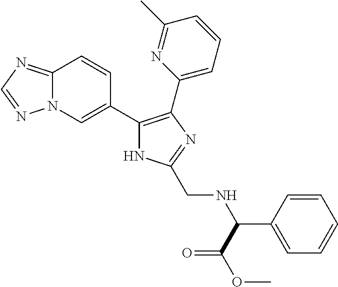 [SMILES: COC(=O)[C@@H](NCc1nc(-c2cccc(C)n2)c(-c2ccc3ncnn3c2)[nH]1)c1ccccc1] | H NMR (MeOD, 400 MHz) δ 9.20 (s, 1H), 8.53 (s, 1H), 7.96-7.83 (m, 3H), 7.58-7.44 (m, 7H), 5.34 (s, 1H), 4.39- 4.26 (m, 2H), 3.80 (s, 3H), 2.69 (s, 3H); m/z found 454.1 [M + H]⁺. |
| 17 | 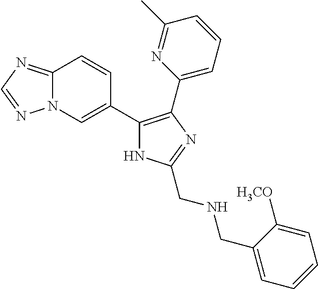 [SMILES: COc1ccccc1CNCc1nc(-c2cccc(C)n2)c(-c2ccc3ncnn3c2)[nH]1] | ¹H NMR (MeOD, 400 MHz) δ 9.18 (s, 1H), 8.54 (s, 1H), 8.15 (t, J = 8.0 Hz, 1H), 7.95-7.90 (m, 1H), 7.87-7.83 (m, 1H), 7.66 (dd, J = 8.0, 11.2 Hz, 2H), 7.50-7.40 (m, 2H), 7.10 (d, J = 8.4 Hz, 1H), 7.02 (dt, J = 0.8, 7.5 Hz, 1H), 4.48 (d, J = 3.6 Hz, 4H), 3.95-3.89 (m, 3H), 2.82 (s, 3H); m/z = 426.6 [M + H]⁺. |
| 18 | 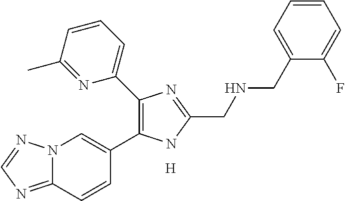 | ¹H NMR (MeOD, 400 MHz) δ 9.19 (s, 1H), 8.54 (s, 1H), 8.17 (t, J = 8.0 Hz, 1H), 7.94-7.83 (m, 2H), 7.70-7.63 (m, 3H), 7.55-7.49 (m, 1H), 7.32-7.23 (m, 2H), 4.56 (s, 2H), 4.59 (s, 2H), 2.84 (s, 3H); m/z = 414.2 [M + H]⁺ |
| 19 | 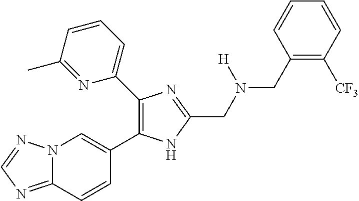 | ¹H NMR (MeOD, 400 MHz) δ 9.18 (s, 1H), 8.42 (s, 1H), 7.88 (dd, J = 1.6, 9.2 Hz, 1H), 7.82-7.75 (m, 2H), 7.70- 7.65 (m, 2H), 7.62 (t, J = 7.6 Hz, 1H), 7.46-7.41 (m, 1H), 7.38 (d, J = 7.6 Hz, 1H), 7.18 (d, J = 7.6 Hz, 1H), 4.04 (s, 2H), 4.00 (s, 2H), 2.51 (s, 3H); m/z = 430.1 [M + H]⁺. |
| 20 | 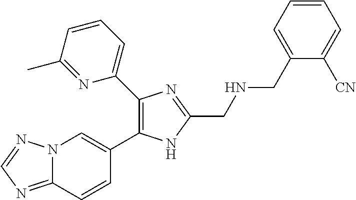 [SMILES: Cc1cccc(-c2nc(CNCc3ccccc3C#N)[nH]c2-c2ccc3ncnn3c2)n1] | ¹H NMR (MeOD, 400 MHz) δ 9.16 (s, 1H), 8.49 (s, 1H), 8.15 (d, J = 8.0 Hz, 1H), 7.91 (t, J = 7.6 Hz, 1H), 7.87- 7.81 (m, 3H), 7.78-7.74 (m, 1H), 7.72-7.67 (m, 1H), 7.52 (d, J = 8.0 Hz, 1H), 7.43 (d, J = 7.6 Hz, 1H), 5.29 (s, 2H), 5.06 (s, 2H), 2.65 (s, 3H); m/z = 421.2 [M + H]⁺. |
| 21 | 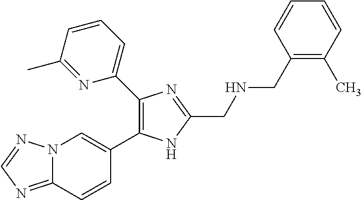 [SMILES: Cc1cccc(-c2nc(CNCc3ccccc3C)[nH]c2-c2ccc3ncnn3c2)n1] | ¹H NMR (MeOD, 400 MHz) δ 9.18 (s, 1H), 8.43 (s, 1H), 7.89 (dd, J = 1.6, 9.2 Hz, 1H), 7.79-7.76 (m, 1H), 7.67 (t, J = 7.8 Hz, 1H), 7.39-7.31 (m, 2H), 7.19 (d, J = 7.8 Hz, 1H), 7.16-7.12 (m, 3H), 4.00 (s, 2H), 3.85 (s, 2H), 2.52 (s, 3H), 2.35 (s, 3H); m/z = 410.2 [M + H]⁺ |
| 22 | 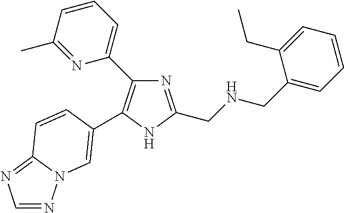 [SMILES: CCc1ccccc1CNCc1nc(-c2cccc(C)n2)c(-c2ccc3ncnn3c2)[nH]1] | ¹H NMR (MeOD, 400 MHz) δ 9.18 (s, 1H), 8.42 (s, 1H), 7.89 (d, J = 9.2 Hz, 1H), 7.79-7.75 (m, 1H), 7.66 (t, J = 7.6 Hz, 1H), 7.40-7.33 (m, 2H), 7.20-7.16 (m, 3H), 7.16- 7.12 (m, 1H), 4.00 (s, 2H), 3.87 (s, 2H), 2.70 (q, J = 7.6 Hz, 2H), 2.51 (s, 3H), 1.18 (t, J = 7.6 Hz, 3H); m/z = 424.2 [M + H]⁺. |
| 23 | 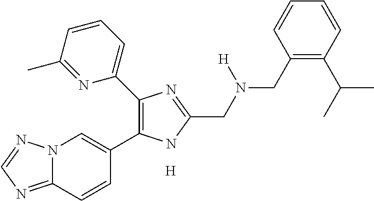 | ¹H NMR (MeOD, 400 MHz) δ 9.53 (s, 1H), 8.55 (s, 1H), 8.00-7.89 (m, 2H), 7.78 (t, J = 7.8 Hz, 1H), 7.56-7.50 (m, 2H), 7.40 (d, J = 3.8 Hz, 2H), 7.31-7.23 (m, 2H), 4.47 (s, 2H), 4.50 (s, 2H), 3.31 (td, J = 6.6, 13.6 Hz, 1H), 2.52 (s, 3H), 1.18 (d, J = 6.8 Hz, 6H); m/z 438.2 [M + H]⁺ |
| 24 | 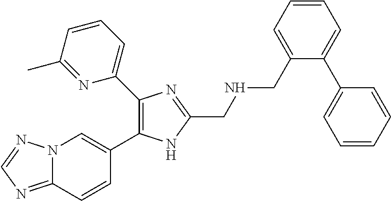 [SMILES: Cc1cccc(-c2nc(CNCc3ccccc3-c3ccccc3)[nH]c2-c2ccc3ncnn3c2)n1] | ¹H NMR (MeOD, 400 MHz) δ 9.11 (s, 1H), 8.42 (s, 1H), 7.86-7.72 (m, 2H), 7.65 (t, J = 7.8 Hz, 1H), 7.56 (d, J = 7.4 Hz, 1H), 7.41-7.23 (m, 7H), 7.22-7.13 (m, 3H), 3.81 (d, J = 17.6 Hz, 4H), 2.51 (s, 3H); m/z = 472.2 [M + H]⁺ |
| 25 | 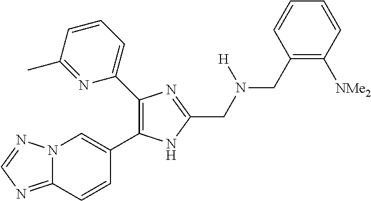 [SMILES: Cc1cccc(-c2nc(CNCc3ccccc3N(C)C)[nH]c2-c2ccc3ncnn3c2)n1] | ¹H NMR (MeOD, 400 MHz) δ 9.15 (s, 1H), 8.42 (s, 1H), 7.87 (d, J = 8.4 Hz, 1H), 7.77 (d, J = 9.2 Hz, 1H), 7.65 (t, J = 7.8 Hz, 1H), 7.35 (dd, J = 3.4, 7.2 Hz, 2H), 7.28-7.12 (m, 3H), 7.04 (t, J = 8.0 Hz 1H), 3.94 (d, J = 6.8 Hz, 4H), 2.65 (s, 6H), 2.52 (s, 3H); m/z = 439.2 [M + H]⁺ |
| 26 | 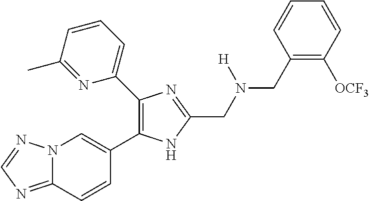 [SMILES: Cc1cccc(-c2nc(CNCc3ccccc3OC(F)(F)F)[nH]c2-c2ccc3ncnn3c2)n1] | ¹H NMR (MeOD, 400 MHz) δ 9.50 (s, 1H), 8.56 (s, 1H), 7.97-7.89 (m, 2H), 7.83-7.76 (m, 2H), 7.61-7.44 (m, 5H), 7.28 (d, J = 7.8 Hz, 1H), 4.45 (s, 4H), 4.52 (s, 4H), 2.53 (s, 3H); m/z = 480.2 [M + H]⁺ |
| 27 | 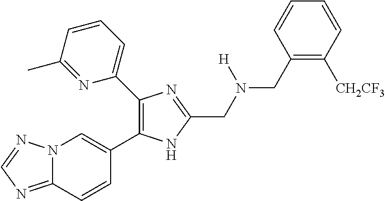 | ¹H NMR (DMSO-d6, 400 MHz) δ 9.49 (s, 1H), 8.56 (s, 1H), 7.97-7.92 (m, 1H), 7.74 (t, J = 7.6 Hz, 1H), 7.66 (s, 1H), 7.52-7.47 (m, 4H), 7.25 (d, J = 7.6 Hz, 1H), 4.53 (s, 2H), 4.47 (s, 2H), 3.92 (q, J = 10.4 Hz, 2H), 2.51 (s, 3H); m/z = 478.2 [M + H]⁺. |
| 28 | 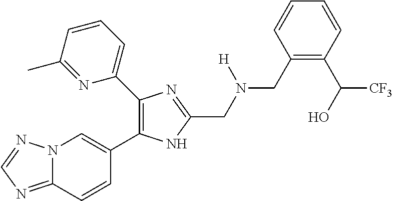 [SMILES: Cc1cccc(-c2nc(CNCc3ccccc3C(O)C(F)(F)F)[nH]c2-c2ccc3ncnn3c2)n1] | ¹H NMR (MeOD, 400 MHz) δ 9.21 (s, 1H), 9.52 (s, 1H), 8.03 (t, J = 8.0 Hz, 1H), 7.89 (s, 2H), 7.63-7.61 (m, 2H), 7.60-7.52 (m, 4H), 5.41 (q, J = 7.6 Hz, 1H), 4.57-4.47 (m, 4H), 2.74 (s, 3H); m/z = 494.2 [M + H]⁺. |
| 29 | 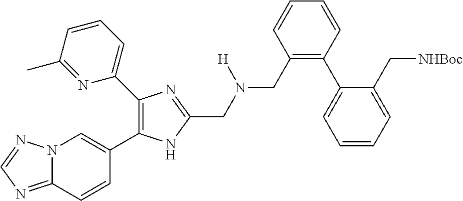 [SMILES: Cc1cccc(-c2nc(CNCc3ccccc3-c3ccccc3CNC(=O)OC(C)(C)C)[nH]c2-c2ccc3ncnn3c2)n1] | ¹H NMR (MeOD, 400 MHz) δ 9.15 (s, 1H), 8.52 (s, 1H), 7.93 (t, J = 7.8 Hz, 1H), 7.87-7.83 (m, 2H), 7.78-7.74 (m, 1H), 7.60-7.49 (m, 3H), 7.45 (d, J = 7.8 Hz, 1H), 7.39- 7.34 (m, 1H), 7.29-7.24 (m, 1H), 7.21-7.17 (m, 2H), 7.15- 7.11 (m, 1H), 4.43-4.26 (m, 4H), 3.96-3.85 (m, 2H), 2.69 (s, 3H), 1.35 (s, 9H); m/z = 601.3 [M + H]⁺ |
| 30 | 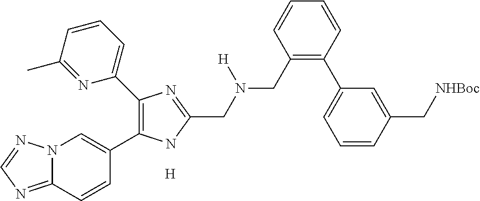 | ¹H NMR (DMSO-d₆, 400 MHz) δ 9.61-9.39 (m, 2H), 8.55 (s, 1H), 7.91-7.71 (m, 4H), 7.54-7.48 (m, 2H), 7.37-7.12 (m, 7H), 4.39 (s, 2H), 4.24 (s, 2H), 4.11 (s, 2H), 2.42 (m, 3H), 1.36 (s, 9H; m/z = 601.3 [M + H]⁺ |
| 31 | 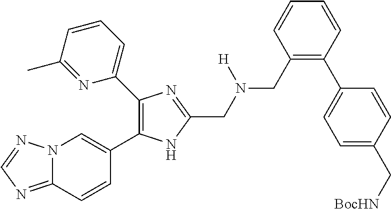 [SMILES: Cc1cccc(-c2nc(CNCc3ccccc3-c3ccc(CNC(=O)OC(C)(C)C)cc3)[nH]c2-c2ccc3ncnn3c2)n1] | ¹H NMR (DMSO-d₆, 400 MHz) δ 9.41 (s, 1H), 8.60-8.51 (m, 1H), 7.93-7.83 (m, 2H), 7.78- 7.71 (m, 2H), 7.54-7.46 (m, 2H), 7.45-7.34 (m, 2H), 7.34-7.26 (m, 3H), 7.26-7.20 (m, 3H), 4.40 (s, 2H), 4.25 (s, 2H), 4.04 (d, J = 6.0 Hz, 2H), 1.37 (s, 9H); m/z = 601.3 [M + H]⁺. |
| 32 | 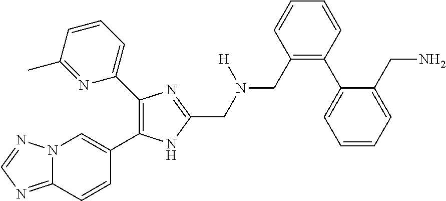 [SMILES: Cc1cccc(-c2nc(CNCc3ccccc3-c3ccccc3CN)[nH]c2-c2ccc3ncnn3c2)n1] | ¹H NMR (MeOD, 400 MHz) δ 9.25 (s, 1H), 8.72 (s, 1H), 8.30-8.23 (m, 1H), 8.01 (d, J = 8.8 Hz, 1H), 7.96-7.86 (m, 2H), 7.76 (d, J = 7.2 Hz, 2H), 7.66-7.51 (m, 3H), 7.44-7.28 (m, 4H), 4.49-4.38 (m, 3H), 4.22 (d, J = 13.8 Hz, 1H), 4.02 (d, J = 13.8 Hz, 1H), 3.84 (d, J = 14.0 Hz, 1H), 2.89 (s, 3H); m/z = 501.2 [M + H]⁺ |
| 33 | 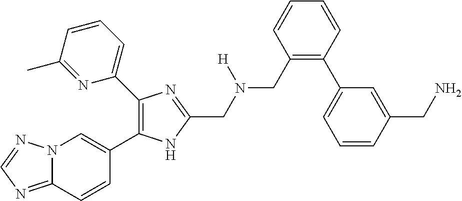 [SMILES: Cc1cccc(-c2nc(CNCc3ccccc3-c3cccc(CN)c3)[nH]c2-c2ccc3ncnn3c2)n1] | ¹H NMR (DMSO-d₆, 400 MHz) δ 10.08 (s, 1H), 9.51 (s, 1H), 8.58 (s, 1H), 8.45 (s, 2H), 7.92-7.81 (m, 3H), 7.57- 7.49 (m, 3H), 7.39-7.29 (m, 4H), 4.39 (s, 2H), 4.31 (s, 2H), 4.10-4.05 (m, 2H), 2.54 (s, 3H); m/z = 501.3 [M + H]⁺ |
| 34 | 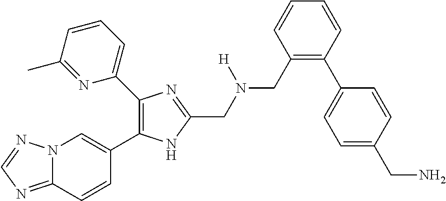 [SMILES: Cc1cccc(-c2nc(CNCc3ccccc3-c3ccc(CN)cc3)[nH]c2-c2ccc3ncnn3c2)n1] | ¹H NMR (MeOD, 400 MHz) δ 9.20 (s, 1H), 8.52 (s, 1H), 7.92-7.83 (m, 3H), 7.78-7.74 (m, 1H), 7.62-7.53 (m, 2H), 7.52-7.35 (m, 7H), 4.51 (s, 2H), 4.32 (s, 2H), 3.97 (s, 2H), 2.66 (s, 3H); m/z = 501.3 [M + H]⁺. |
| 35 | 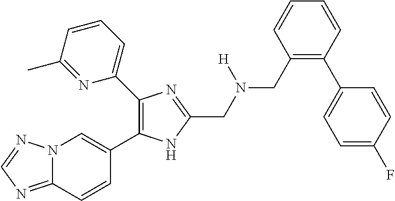 [SMILES: Cc1cccc(-c2nc(CNCc3ccccc3-c3ccc(F)cc3)[nH]c2-c2ccc3ncnn3c2)n1] | ¹H NMR (DMSO, 400 MHz) δ 9.39 (s, 1H), 8.53 (s, 1H), 7.89-7.71 (m, 4H), 7.49 (dtd, J = 1.2, 7.2, 18.0 Hz, 2H), 7.40 (d, J = 8.0 Hz, 1H), 7.36-7.27 (m, 3H), 7.24 (d, J = 7.2 Hz, 1H), 7.14-7.08 (m, 2H), 4.36 (s, 2H), 4.23 (s, 2H), 2.50 (s, 3H); m/z = 490.1 [M + H]⁺ |
| 36 | 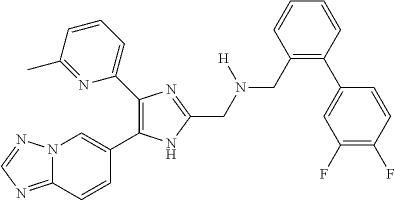 [SMILES: Cc1cccc(-c2nc(CNCc3ccccc3-c3ccc(F)c(F)c3)[nH]c2-c2ccc3ncnn3c2)n1] | ¹H NMR (DMSO, 400 MHz) δ 9.41 (s, 1H), 8.52 (s, 1H), 7.84 (dq, J = 1.2, 9.2 Hz, 2H), 7.76-7.71 (m, 2H), 7.56- 7.29 (m, 6H), 7.22 (d, J = 7.2 Hz, 1H), 7.10 (s, 1H), 4.38 (s, 2H), 4.23 (s, 2H), 2.50 (s, 3H); m/z = 508.2 [M + H]⁺ |
| 37 | 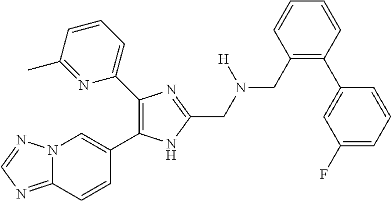 [SMILES: Cc1cccc(-c2nc(CNCc3ccccc3-c3cccc(F)c3)[nH]c2-c2ccc3ncnn3c2)n1] | ¹H NMR (MeOD, 400 MHz): δ 9.13 (s, 1H), 8.52 (s, 1H), 8.01 (s, 1H), 7.89-7.86 (m, 1H), 7.82-7.79 (m, 1H), 7.73- 7.70 (m, 1H), 7.55 (br d, J = 7.6 Hz, 4H), 7.37-7.27 (m, 2H), 7.10-7.04 (m, 2H), 6.92-6.86 (m, 1H), 4.52 (s, 2H), 4.32 (s, 2H), 2.72 (s, 3H); m/z = 490.2 [M + H]⁺ |
| 38 | 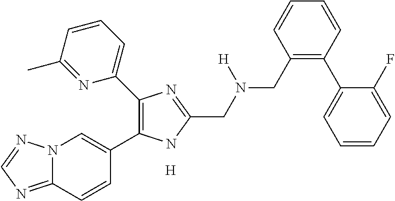 | ¹H NMR (DMSO-d₆, 400 MHz) δ 12.42 (s, 1H), 9.69-9.46 (m, 1H), 8.50 (s, 1H), 7.93 (d, J = 9.4 Hz, 1H), 7.83 (s, 1H), 7.70 (d, J = 6.8 Hz, 2H), 7.43 (t, J = 7.6 Hz, 1H), 7.36-7.31 (m, 4H), 7.24-7.14 (m, 4H), 3.70 (s, 2H), 3.63 (s, 2H), 2.51 (s, 3H); m/z = 490.3 [M + H]⁺ |
| 39 | 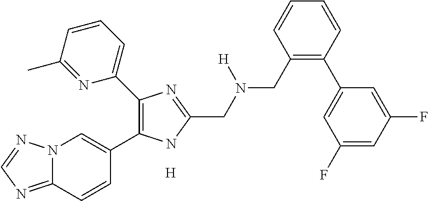 | ¹H NMR (DMSO-d₆, 400 MHz) δ 12.47 (s, 1H), 9.61-9.48 (m, 1H), 8.49 (s, 1H), 7.95 (dd, J = 1.6, 9.4 Hz, 1H), 7.82 (s, 1H), 7.70-7.63 (m, 2H), 7.42 (t, J = 6.8 Hz, 1H), 7.35 (t, J = 7.0 Hz, 1H), 7.28-7.25 (m, 1H), 7.19 (d, J = 6.8 Hz, 4H), 3.78 (s, 2H), 3.71 (s, 2H), 2.49 (s, 3H); m/z = 508.3 [M + H]⁺ |
| 40 | 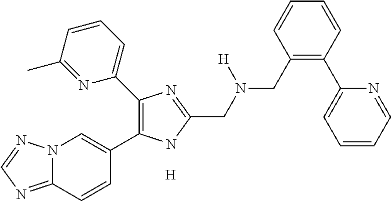 | ¹H NMR (MeOD, 400 MHz) δ 9.15 (s, 1H), 8.54 (d, J = 4.4 Hz, 1H), 8.44 (s, 1H), 7.88-7.76 (m, 3H), 7.67 (t, J = 7.8 Hz, 1H), 7.58 (dd, J = 7.8, 11.0 Hz, 2H), 7.46-7.40 (m, 4H), 7.35 (d, J = 7.8 Hz, 1H), 7.26-7.18 (m, 2H), 3.97 (s, 2H), 3.82 (s, 2H), 2.53 (s, 3H); m/z = 473.3 [M + H]⁺ |
| 41 | 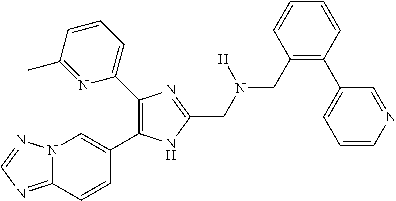 [SMILES: Cc1cccc(-c2nc(CNCc3ccccc3-c3cccnc3)[nH]c2-c2ccc3ncnn3c2)n1] | ¹H NMR (MeOD, 400 MHz) δ 9.16 (s, 1H), 8.53 (d, J = 1.6 Hz, 1H), 8.43 (s, 1H), 8.39 (dd, J = 1.6, 5.2 Hz, 1H), 7.90-7.81 (m, 2H), 7.80-7.75 (m, 1H), 7.70-7.59 (m, 2H), 7.44 (t, J = 7.5 Hz, 1H), 7.40-7.31 (m, 3H), 7.25 (dd, J = 1.2, 7.5 Hz, 1H), 7.19 (d, J = 7.6 Hz, 1H), 3.87 (s, 2H), 3.76 (s, 2H), 2.52 (s, 3H); m/z = 473.2 [M + H]⁺. |
| 42 | 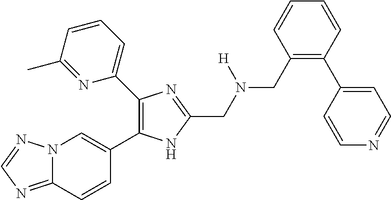 [SMILES: Cc1cccc(-c2nc(CNCc3ccccc3-c3ccncc3)[nH]c2-c2ccc3ncnn3c2)n1] | ¹H NMR (MeOD, 400 MHz) δ 9.12 (s, 1H), 8.42 (s, 3H), 7.89-7.71 (m, 2H), 7.70-7.59 (m, 2H), 7.43 (s, 3H), 7.34 (dd, J = 7.6, 16.4 Hz, 2H), 7.24 (d, J = 7.2 Hz, 1H), 7.16 (d, J = 7.2 Hz, 1H), 3.88 (s, 2H), 3.76 (s, 2H), 2.51 (s, 3H); m/z = 473.2 [M + H]⁺. |
| 43 | 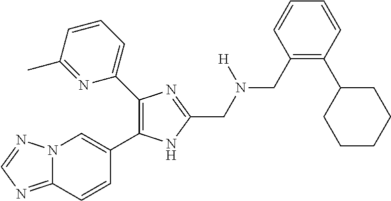 [SMILES: Cc1cccc(-c2nc(CNCc3ccccc3C3CCCCC3)[nH]c2-c2ccc3ncnn3c2)n1] | ¹H NMR (MeOD, 400 MHz) δ 9.26 (s, 1H), 8.51 (s, 1H), 7.98-7.90 (m, 2H), 7.89-7.85 (m, 1H), 7.58 (d, J = 8.0 Hz, 1H), 7.50 (d, J = 7.6 Hz, 1H), 7.45 (d, J = 7.6 Hz, 1H), 7.43-7.38 (m, 2H), 7.34-7.23 (m, 1H), 4.53 (s, 4H), 2.69 (s, 3H), 1.72 (t, J = 13.6 Hz, 4H), 1.59-1.38 (m, 4H), 1.21 (s, 2H), 1.30-1.12 (m, 1H); m/z = 478.3 [M + H]⁺ |
| 44 | 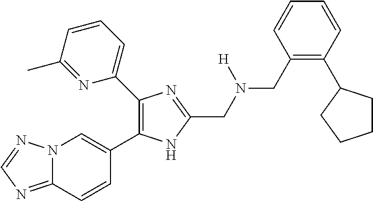 [SMILES: Cc1cccc(-c2nc(CNCc3ccccc3C3CCCC3)[nH]c2-c2ccc3ncnn3c2)n1] | ¹H NMR (MeOD, 400 MHz) δ 9.21 (s, 1H), 8.44 (s, 1H), 7.91 (dd, J = 1.8, 9.2 Hz, 1H), 7.79 (d, J = 9.4 Hz, 1H), 7.67 (t, J = 7.8 Hz, 1H), 7.39 (d, J = 8.0 Hz, 1H), 7.31 (dd, J = 7.2, 14.6 Hz, 2H), 7.25-7.17 (m, 2H), 7.16-7.10 (m, 1H), 4.00 (s, 2H), 3.89 (s, 2H), 3.18 (d, J = 8.4 Hz, 1H), 2.53 (s, 3H), 1.96-2.02 (m, 2H), 1.74-1.80 (m, 2H), 1.54-1.62 (m, 4H); m/z = 464.3 [M + H]+ |
| 45 | 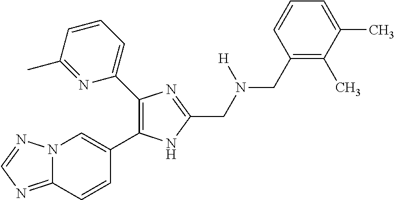 [SMILES: Cc1cccc(-c2nc(CNCc3cccc(C)c3C)[nH]c2-c2ccc3ncnn3c2)n1] | ¹H NMR (MeOD, 400 MHz) δ 9.11 (s, 1H), 8.43 (s, 1H), 7.85 (d, J = 9.2 Hz, 1H), 7.78 (d, J = 9.2 Hz, 1H), 7.69 (t, J = 7.6 Hz, 1H), 7.35 (d. J = 7.6 Hz, 1H), 7.20 (d, J = 7.6 Hz, 1H), 7.14-7.10 (m, 1H), 7.02-7.01 (m, 2H), 3.99 (s, 2H), 3.84 (s, 2H), 2.50 (s, 3H), 2.23 (s, 6H); m/z = 424.1 [M + H]⁺. |
| 46 | 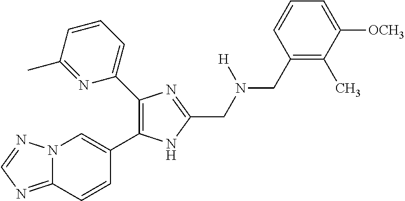 [SMILES: COc1cccc(CNCc2nc(-c3cccc(C)n3)c(-c3ccc4ncnn4c3)[nH]2)c1C] | ¹H NMR (MeOD, 400 MHz) δ 9.17 (s, 1H), 8.42 (s, 1H), 7.89 (d, J = 1.6 Hz, 1H), 7.87 (d, J = 1.6 Hz, 1H), 7.78- 7.66 (m, 1H), 7.35 (d, J = 7.6 Hz, 1H), 7.17 (d, J = 7.6 Hz, 1H), 7.13-7.09 (m, 1H), 6.92 (d, J = 7.6 Hz, 1H), 6.81 (d, J = 8.4 Hz, 1H), 3.98 (s, 2H), 3.84 (s, 2H), 3.78 (s, 3H), 2.51 (s, 3H), 2.19 (s, 3H); m/z = 440.2 [M + H]⁺. |
| 47 | 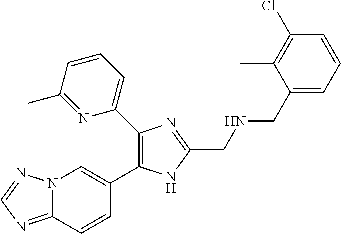 [SMILES: Cc1cccc(-c2nc(CNCc3cccc(Cl)c3C)[nH]c2-c2ccc3ncnn3c2)n1] | 1H NMR (CDCl3, 400 MHz): δ 9.18 (s, 1H), 8.44 (s, 1H), 7.94-7.85 (m, 1H), 7.84-7.74 (m, 1H), 7.73-7.63 (m, 1H), 7.44-7.07 (m, 6H), 4.00 (s, 2H), 3.90 (s, 2H), 2.53 (s, 3H), 2.41 (s, 3H); m/z = 444.0 [M + H]+ |
| 48 | 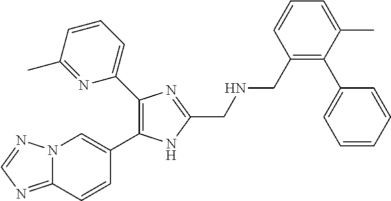 [SMILES: Cc1cccc(-c2nc(CNCc3cccc(C)c3-c3ccccc3)[nH]c2-c2ccc3ncnn3c2)n1] | ¹H NMR (MeOD, 400 MHz): δ 9.15 (s, 1H), 8.46 (s, 1H), 7.89-7.84 (m, 1H), 7.82-7.78 (m, 1H), 7.69 (t, J = 7.6 Hz, 1H), 7.42-7.25 (m, 5H), 7.23-7.18 (m, 3H), 7.17-7.12 (m, 2H), 3.81 (s, 2H), 3.57 (s, 2H), 2.55 (s, 3H), 1.99 (s, 3H); m/z = 486.0 [M + H]⁺ |
| 49 | 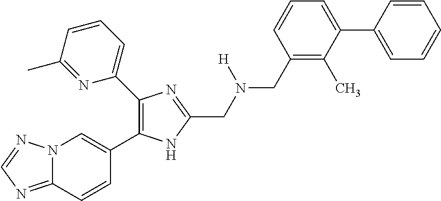 [SMILES: Cc1cccc(-c2nc(CNCc3cccc(-c4ccccc4)c3C)[nH]c2-c2ccc3ncnn3c2)n1] | ¹H NMR (MeOD, 400 MHz) δ 9.16 (s, 1H), 8.42 (s, 1H), 7.87 (dd, J = 1.6, 9.6 Hz, 1H), 7.65 (t, J = 7.6 Hz, 1H), 7.42-7.26 (m, 5H), 7.23-7.13 (m, 4H), 7.07 (d, J = 7.6 Hz, 1H), 7.81-6.59 (m, 1H), 4.03 (s, 2H), 3.92 (s, 2H), 2.50 (s, 3H), 2.21 (s, 3H); m/z = 486.3 [M + H]⁺. |
| 50 | 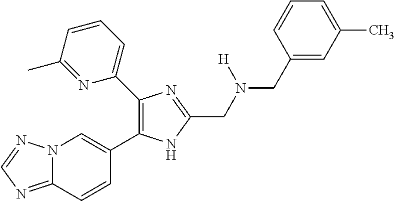 [SMILES: Cc1cccc(CNCc2nc(-c3cccc(C)n3)c(-c3ccc4ncnn4c3)[nH]2)c1] | ¹H NMR (MeOD, 400 MHz) δ 9.21 (s, 1H), 8.53 (s, 1H), 8.03 (t, J = 8.0 Hz, 1H), 7.89 (s, 2H), 7.61 (d, J = 8.0 Hz, 1H), 7.54 (d, J = 7.6 Hz, 1H), 7.41-7.28 (m, 4H), 4.46 (s, 2H), 4.43 (s, 2H), 2.74 (s, 3H), 2.39 (s, 3H); m/z = 410.1 [M + H]⁺. |
| 51 | 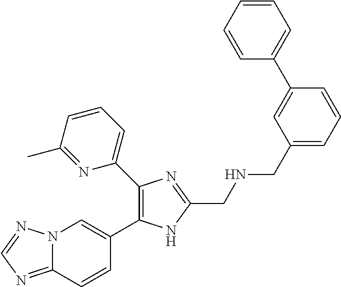 [SMILES: Cc1cccc(-c2nc(CNCc3cccc(-c4ccccc4)c3)[nH]c2-c2ccc3ncnn3c2)n1] | ¹H NMR (MeOD, 400 MHz): δ 9.18 (d, J = 1.6 Hz, 1H), 8.53-8.50 (m, 1H), 8.03-7.97 (m, 1H), 7.89-7.80 (m, 4H), 7.75-7.70 (m, 1H), 7.65-7.60 (m, 2H), 7.59-7.49 (m, 5H), 7.45-7.38 (m, 2H), 7.37-7.32 (m, 1H), 4.55 (s, 2H), 4.51 (s, 2H), 2.72 (s, 3H); m/z = 472.2 [M + H]⁺ |
| 52 | 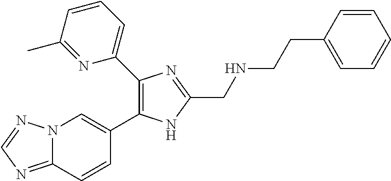 [SMILES: Cc1cccc(-c2nc(CNCCc3ccccc3)[nH]c2-c2ccc3ncnn3c2)n1] | ¹H NMR (MeOD, 400 MHz): δ 9.16 (s, 1H), 8.42 (s, 1H), 7.90-7.83 (m, 1H), 7.79-7.73 (m, 1H), 7.70-7.62 (m, 1H), 7.39-7.33 (m, 1H), 7.30-7.12 (m, 7H), 3.94 (s, 2H), 2.93 (s, 2H), 2.83 (s, 2H), 2.51 (s, 3H); m/z = 410.1 [M + H]⁺ |
| 53 | 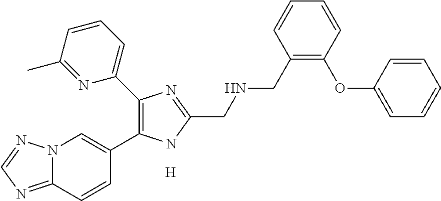 | ¹H NMR (MeOD, 400 MHz) δ 9.09 (s, 1H), 8.43 (s, 1H), 7.84-7.80 (m, 1H), 7.76-7.73 (m, 1H), 7.65 (t, J = 7.8 Hz, 1H), 7.50 (d, J = 7.8 Hz, 1H), 7.32 (d, J = 7.8 Hz, 1H), 7.27-7.12 (m, 6H), 7.02-6.98 (m, 1H), 6.89 (d, J = 7.8 Hz, 2H), 6.84 (d, J = 8.2 Hz, 1H), 3.96 (s, 2H), 3.87 (s, 2H), 2.52 (s, 3H); m/z = 488.3 [M + H]⁺ |
| 54 | 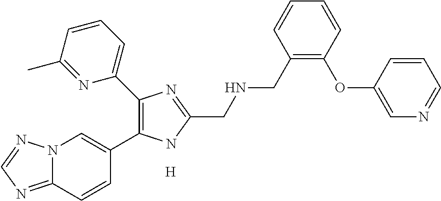 | ¹H NMR (MeOD, 400 MHz) δ 9.11 (s, 1H), 8.43 (s, 1H), 8.24 (d, J = 2.6 Hz, 1H), 8.19 (dd, J = 1.4, 4.6 Hz, 1H), 7.84-7.81 (m, 1H), 7.77-7.72 (m, 1H), 7.65 (t, J = 7.8 Hz, 1H), 7.56 (d, J = 6.1 Hz, 1H), 7.36-7.27 (m, 4H), 7.25- 7.17 (m, 2H), 6.92 (d, J = 8.2 Hz, 1H), 3.96 (s, 2H), 3.88 (s, 2H), 2.52 (s, 3H); m/z = 489.3 [M + H]⁺ |
| 55 | 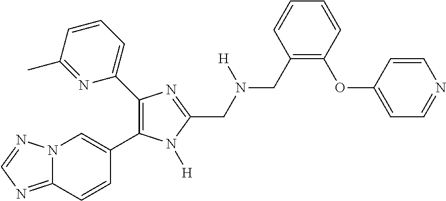 [SMILES: Cc1cccc(-c2nc(CNCc3ccccc3Oc3ccncc3)[nH]c2-c2ccc3ncnn3c2)n1] | ¹H NMR (MeOD, 400 MHz) δ 9.13 (s, 1H), 8.43 (s, 1H), 7.89-7.84 (m, 3H), 7.79-7.76 (m, 1H), 7.72-7.65 (m, 2H), 7.56 (t, J = 7.2 Hz, 1H), 7.48 (t, J = 6.8 Hz, 1H), 7.39 (d, J = 7.4 Hz, 2H), 7.19 (d, J = 7.8 Hz, 1H), 6.43 (d, J = 7.4 Hz, 2H), 3.90 (s, 2H), 3.71 (s, 2H), 2.53 (s, 3H); m/z = 489.3 [M + H]⁺ |
| 56 | 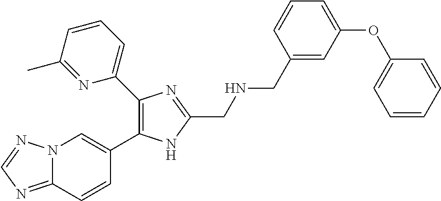 [SMILES: Cc1cccc(-c2nc(CNCc3cccc(Oc4ccccc4)c3)[nH]c2-c2ccc3ncnn3c2)n1] | ¹H NMR (MeOD, 400 MHz): δ 9.10 (s, 1H), 8.36 (s, 1H), 7.83-7.76 (m, 1H), 7.73-7.66 (m, 1H), 7.59 (t, J = 7.6 Hz, 1H), 7.32-7.19 (m, 4H), 7.15-7.04 (m, 2H), 7.03-6.95 (m, 2H), 6.92-6.84 (m, 2H), 6.82-6.75 (m, 1H), 3.88 (s, 2H), 3.78 (s, 2H), 2.45 (s, 3H); m/z = 488.3 [M + H]⁺ |
| 57 | 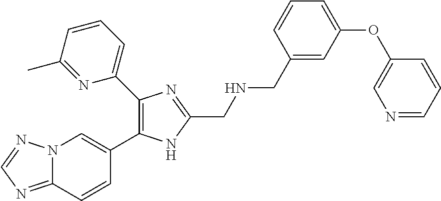 [SMILES: Cc1cccc(-c2nc(CNCc3cccc(Oc4cccnc4)c3)[nH]c2-c2ccc3ncnn3c2)n1] | ¹H NMR (MeOD, 400 MHz): δ 9.11-9.08 (m, 1H), 8.36 (s, 1H), 8.19 (s, 2H), 7.82-7.78 (m, 2H), 7.71-7.67 (m, 1H), 7.62-7.56 (m, 1H), 7.35-7.26 (m, 6H), 7.17-7.14 (m, 1H), 7.13-7.09 (m, 1H), 7.08-7.05 (m, 1H), 6.89-6.85 (m, 1H), 3.89 (s, 2H), 3.81 (s, 2H), 2.44 (s, 3H); m/z = 489.3 [M + H]⁺ |
| 58 | 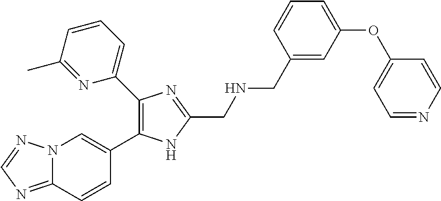 [SMILES: Cc1cccc(-c2nc(CNCc3cccc(Oc4ccncc4)c3)[nH]c2-c2ccc3ncnn3c2)n1] | ¹H NMR (MeOD, 400 MHz): δ 9.19 (s, 1H), 8.43 (s, 1H), 8.40-8.32 (m, 2H), 7.88 (dd, J = 9.2, 1.6 Hz, 1H), 7.81- 7.74 (m, 1H), 7.68 (t, J = 7.6 Hz, 1H), 7.50-7.42 (m, 1H), 7.40-7.32 (m, 2H), 7.27-7.16 (m, 2H), 7.05 (dd, J = 8.0, 1.6 Hz, 1H), 6.97-6.90 (m, 2H), 4.01 (s, 2H), 3.95 (s, 2H), 2.53 (s, 3H); m/z = 489.3 [M + H]⁺ |
| 59 | 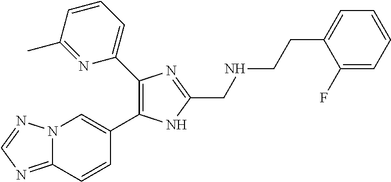 [SMILES: Cc1cccc(-c2nc(CNCCc3ccccc3F)[nH]c2-c2ccc3ncnn3c2)n1] | ¹H NMR (MeOD, 400 MHz) δ 9.17 (s, 1H), 8.43 (s, 1H), 7.88 (d, J = 9.2 Hz, 1H), 7.77 (d, J = 9.2 Hz, 1H), 7.67 (t, J = 7.6 Hz, 1H), 7.37 (d, J = 8.0 Hz, 1H), 7.30-7.26 (m, 1H), 7.24-7.17 (m, 2H), 7.10-7.01 (m, 1H), 3.97 (s, 2H), 2.95-2.92 (m, 4H), 2.52 (s, 3H); m/z = 428.2 [M + H]⁺. |
| 60 | 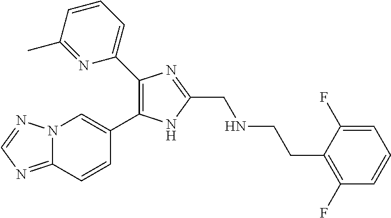 [SMILES: Cc1cccc(-c2nc(CNCCc3c(F)cccc3F)[nH]c2-c2ccc3ncnn3c2)n1] | ¹H NMR (MeOD, 400 MHz): δ 9.16 (s, 1H), 8.41 (s, 1H), 7.89-7.84 (m, 1H), 7.78-7.73 (m, 1H), 7.65 (t, J = 8.0 Hz, 1H), 7.35 (d, J = 8.0 Hz, 1H), 7.27-7.14 (m, 2H), 6.95- 6.88 (m, 2H), 3.96 (s, 2H), 2.97-2.84 (m, 4H), 2.50 (s, 3H); m/z = 446.2 [M + H]⁺ |
| 61 | 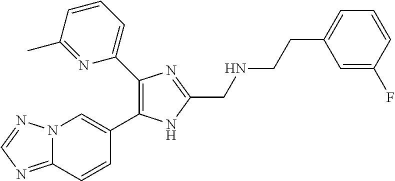 [SMILES: Cc1cccc(-c2nc(CNCCc3cccc(F)c3)[nH]c2-c2ccc3ncnn3c2)n1] | ¹H NMR (MeOD, 400 MHz) δ 9.17 (s, 1H), 8.42 (s, 1H), 7.88 (dd, J = 1.6, 9.2 Hz, 1H), 7.77 (d, J = 9.2 Hz, 1H), 7.66 (t, J = 7.6 Hz, 1H), 7.36 (d, J = 7.6 Hz, 1H), 7.31- 7.24 (m, 1H), 7.18 (d, J = 7.6 Hz, 1H), 7.04 (d, J = 7.6 Hz, 1H), 6.98 (d, J = 10.0 Hz, 1H), 6.90 (dt, J = 2.4, 8.4 Hz, 1H), 3.96 (s, 2H), 3.01-2.90 (m, 2H), 2.89-2.81 (m, 2H), 2.52 (s, 3H); m/z = 428.3 [M + H]⁺ |
| 62 | 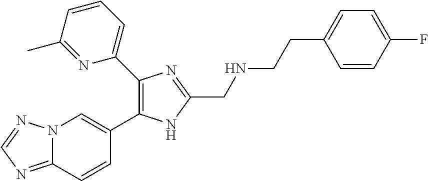 [SMILES: Cc1cccc(-c2nc(CNCCc3ccc(F)cc3)[nH]c2-c2ccc3ncnn3c2)n1] | ¹H NMR (MeOD, 400 MHz) δ 9.17 (s, 1H), 8.43 (s, 1H), 7.88 (dd, J = 1.6, 9.2 Hz, 1H), 7.77 (d, J = 9.2 Hz, 1H), 7.67 (t, J = 7.6 Hz, 1H), 7.36 (d, J = 7.6 Hz, 1H), 7.27-7.16 (m, 3H), 6.99 (t, J = 8.8 Hz, 2H), 3.96 (s, 2H), 2.96-2.89 (m, 2H), 2.87-2.80 (m, 2H), 2.52 (s, 3H); m/z = 427.19 [M + H]⁺. |
| 63 | 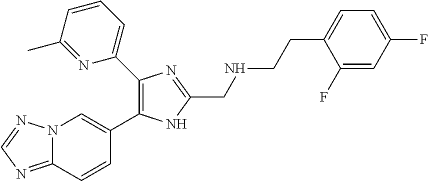 [SMILES: Cc1cccc(-c2nc(CNCCc3ccc(F)cc3F)[nH]c2-c2ccc3ncnn3c2)n1] | ¹H NMR (MeOD, 400 MHz) δ 9.18 (s, 1H), 8.43 (s, 1H), 7.88 (d, J = 9.2 Hz, 1H), 7.77 (d, J = 9.2 Hz, 1H), 7.65 (d, J = 8.0 Hz, 1H), 7.37 (d, J = 8.0 Hz, 1H), 7.33-7.27 (m, 1H), 7.18 (d, J = 7.6 Hz, 1H), 6.88-6.86 (m, 2H), 3.96 (s, 2H), 2.92-2.85 (m, 4H), 2.52 (s, 3H); m/z = 446.2 [M + H]⁺. |
| 64 | 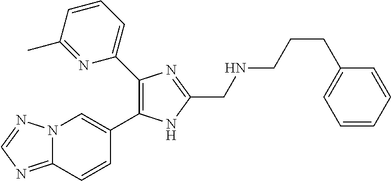 [SMILES: Cc1cccc(-c2nc(CNCCCc3ccccc3)[nH]c2-c2ccc3ncnn3c2)n1] | ¹H NMR (MeOD, 400 MHz) δ 9.19 (s, 1H), 8.43 (s, 1H), 7.89 (dd, J = 1.2, 9.2 Hz, 1H), 7.77 (d, J = 9.2 Hz, 1H), 7.67 (t, J = 7.6 Hz, 1H), 7.38 (d, J = 8.0 Hz, 1H), 7.27- 7.10 (m, 6H), 4.07 (s, 2H), 2.93-2.79 (m, 2H), 2.70 (t, J = 7.6 Hz, 2H), 2.52 (s, 3H), 1.92-1.98 (m, 2H); m/z = 424.3 [M + H]⁺. |
| 65 | 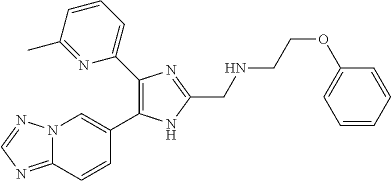 [SMILES: Cc1cccc(-c2nc(CNCCOc3ccccc3)[nH]c2-c2ccc3ncnn3c2)n1] | ¹H NMR (MeOD, 400 MHz) δ 9.15 (s, 1H), 8.42 (s, 1H), 7.90-7.83 (m, 1H), 7.79-7.72 (m, 1H), 7.65 (t, J = 7.6 Hz, 1H), 7.35 (d, J = 7.6 Hz, 1H), 7.27-7.14 (m, 3H), 6.96- 6.84 (m, 3H), 4.10 (t, J = 5.2 Hz, 2H), 4.03 (s, 2H), 3.09 (t, J = 5.2 Hz, 2H), 2.52 (s, 3H)′ m/z = 426.3 [M + H]⁺. |
| 66 | 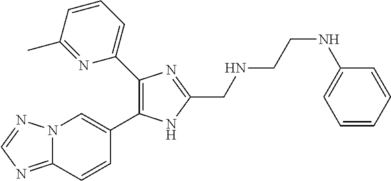 [SMILES: Cc1cccc(-c2nc(CNCCNc3ccccc3)[nH]c2-c2ccc3ncnn3c2)n1] | ¹H NMR (MeOD, 400 MHz) δ .16 (s, 1H), 8.43 (s, 1H), 7.89-7.84 (m, 1H), 7.79-7.74 (m, 1H), 7.67 (t, J = 7.6 Hz, 1H), 7.36 (d, J = 8.0 Hz, 1H), 7.19 (d, J = 7.6 Hz, 1H), 7.08 (t, J = 8.0 Hz, 2H), 6.66 (d, J = 7.6 Hz, 2H), 6.61 (t, J = 7.2 Hz, 1H), 4.11 (s, 2H), 3.36-3.32 (m, 2H), 3.06 (t, J = 6.0 Hz, 2H), 2.53 (s, 3H); m/z = 425.2 [M + H]⁺. |
| 67 | 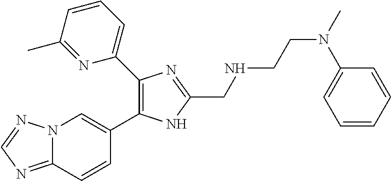 [SMILES: Cc1cccc(-c2nc(CNCCN(C)c3ccccc3)[nH]c2-c2ccc3ncnn3c2)n1] | ¹H NMR (MeOD, 400 MHz) δ 9.16 (s, 1H), 8.43 (s, 1H), 7.85 (d, J = 9.2 Hz, 1H), 7.76 (d, J = 9.2 Hz, 1H), 7.67 (t, J = 7.6 Hz, 1H), 7.35 (d, J = 7.6 Hz, 1H), 7.19 (d, J = 7.6 Hz, 1H), 7.14 (t, J = 8.0 Hz, 2H), 6.77 (d, J = 8.0 Hz, 2H), 6.62 (t, J = 7.2 Hz, 1H), 3.97 (s, 2H), 3.48 (t, J = 6.8 Hz, 2H), 2.93 (s, 3H), 2.88 (t, J = 6.8 Hz, 2H), 2.52 (s, 3H); m/z = 439.3 [M + H]⁺. |
| 68 | 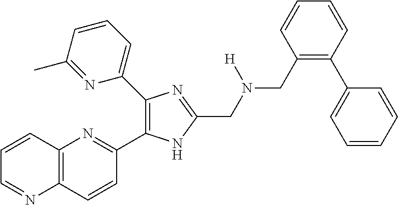 [SMILES: Cc1cccc(-c2nc(CNCc3ccccc3-c3ccccc3)[nH]c2-c2ccc3ncccc3n2)n1] | ¹H NMR (MeOD, 400 MHz) δ 9.17 (dd, J = 1.6, 4.4 Hz, 1H), 8.93 (d, J = 8.4 Hz, 1H), 8.79 (d, J = 9.2 Hz, 1H), 8.58-8.51 (m, 2H), 8.41 (d, J = 7.6 Hz, 1H), 8.07 (dd, J = 4.4, 8.8 Hz, 1H), 7.90 (d, J = 1.6 Hz, 1H), 7.83-7.76 (m, 1H), 7.55 (J = 1.6, 7.2 Hz, 2H), 7.36 (dd, J = 1.6, 7.2 Hz, 2H), 7.31-7.21 (m, 4H), 7.16-7.08 (m, 1H), 4.64 (s, 2H), 4.42 (s, 2H), 3.17 (s, 3H); m/z = 483.3 [M + H]⁺. |
| 69 | 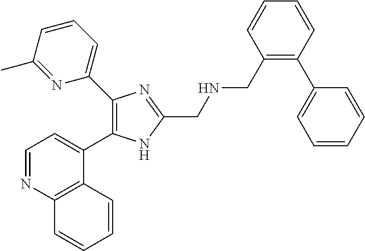 [SMILES: Cc1cccc(-c2nc(CNCc3ccccc3-c3ccccc3)[nH]c2-c2ccnc3ccccc23)n1] | ¹H NMR (MeOD, 400 MHz): δ 9.25 (d, J = 5.6 Hz, 1H), 8.37-8.32 (m, 1H), 8.27-8.17 (m, 2H), 8.09 (d, J = 5.6 Hz, 1H), 7.97-7.80 (m, 3H), 7.61-7.51 (m, 3H), 7.43-7.27 (m, 6H), 7.21-7.14 (m, 1H), 4.62 (s, 2H), 4.43 (s, 2H), 2.67 (s, 3H); m/z = 482.0 [M + H]⁺ |
| 70 | 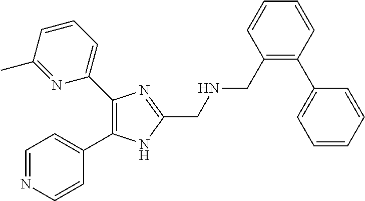 [SMILES: Cc1cccc(-c2nc(CNCc3ccccc3-c3ccccc3)[nH]c2-c2ccncc2)n1] | ¹H NMR (MeOD, 400 MHz): δ 8.79 (d, J = 7.2 Hz, 2H), 8.29-8.23 (m, 3H), 7.82-7.73 (m, 3H), 7.56-7.47 (m, 2H), 7.37-7.27 (m, 5H), 7.23-7.17 (m, 1H), 4.55 (s, 2H), 4.34 (s, 2H), 2.78 (s, 3H); m/z = 432.0 [M + H]⁺ |
| 71 | 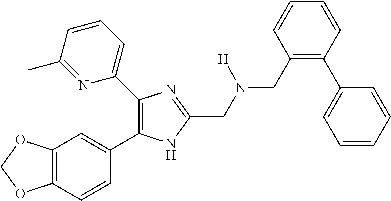 [SMILES: Cc1cccc(-c2nc(CNCc3ccccc3-c3ccccc3)[nH]c2-c2ccc3c(c2)OCO3)n1] | ¹H NMR (MeOD, 400 MHz) δ 8.29 (t, J = 8.0 Hz, 1H), 7.79-7.77 (m, 1H), 7.70 (t, J = 8.0 Hz, 2H), 7.59-7.53 (m, 2H), 7.79-7.37 (m, 1H), 7.30-7.29 (m, 4H), 7.19-7.18 (m, 1H), 7.03-7.00 (m, 3H), 6.11 (s, 2H), 4.54 (s, 2H), 4.31 (s, 2H), 2.87 (s, 3H); m/z = 475.3 [M + H]⁺. |
| 72 | 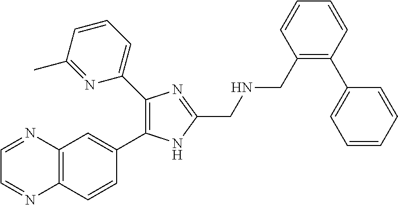 [SMILES: Cc1cccc(-c2nc(CNCc3ccccc3-c3ccccc3)[nH]c2-c2ccc3nccnc3c2)n1] | ¹H NMR (MeOD, 400 MHz) δ 8.99 (s, 2H), 8.30-8.25 (m, 3H), 7.95 (d, J = 8.4 Hz, 1H), 7.79 (t, J = 7.2 Hz, 2H), 7.69 (d, J = 8.4 Hz, 1H), 7.58-7.49 (m, 3H), 7.38 (d, J = 6.4 Hz, 2H), 7.31-7.30 (m, 4H), 7.20-7.13 (m, 1H), 4.57 (s, 2H), 4.37 (s, 2H), 2.87 (s, 3H); m/z = 483.3 [M + H]⁺. |
| 73 | 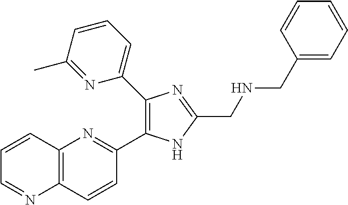 [SMILES: Cc1cccc(-c2nc(CNCc3ccccc3)[nH]c2-c2ccc3ncccc3n2)n1] | ¹H NMR (MeOD, 400 MHz) δ 9.18 (dd, J = 1.4, 4.6 Hz, 1H), 8.93 (d, J = 8.8 Hz, 1H), 8.81 (d, J = 9.0 Hz, 1H), 8.60-8.52 (m, 2H), 8.08 (dd, J = 4.4, 8.8 Hz, 1H), 7.90 (d, J = 8.4 Hz, 1H), 7.62 (dd, J = 1.8, 7.6 Hz, 2H), 7.53-7.44 (m, 4H), 4.62 (s, 2H), 4.55 (s, 2H), 3.16 (s, 3H); m/z = 407.3 [M + H]⁺ |
| 74 | 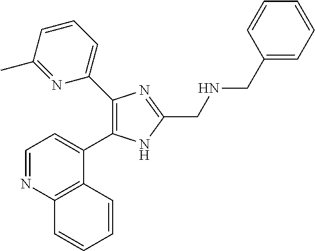 [SMILES: Cc1cccc(-c2nc(CNCc3ccccc3)[nH]c2-c2ccnc3ccccc23)n1] | ¹H NMR (MeOD, 400 MHz): δ 9.23 (d, J = 5.6 Hz, 1H), 8.32 (d, J = 8.8 Hz, 2H), 8.22-8.14 (m, 2H), 7.96 (t, J = 8.0 Hz, 1H), 7.91-7.85 (m, 1H), 7.66-7.60 (m, 2H), 7.59- 7.54 (m, 1H), 7.52-7.42 (m, 3H), 7.35 (d, J = 8.4 Hz, 1H), 4.58 (s, 2H), 4.57 (s, 2H), 2.71 (s, 3H); m/z = 406.0 [M + H]⁺ |
| 75 | 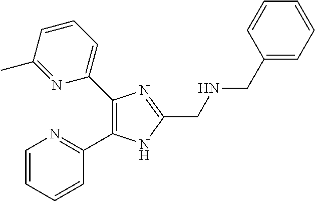 [SMILES: Cc1cccc(-c2nc(CNCc3ccccc3)[nH]c2-c2ccccn2)n1] | ¹H NMR (MeOD, 400 MHz) δ 8.95 (d, J = 4.8 Hz, 1H), 8.46 (d, J = 7.2 Hz, 2H), 8.42-8.33 (m, 2H), 7.78 (ddd, J = 1.2, 5.6, 7.2 Hz, 1H), 7.74 (d, J = 7.6 Hz, 1H), 7.60 (dd, J = 2.0, 7.6 Hz, 2H), 7.53-7.46 (m, 3H), 4.57 (s, 2H), 4.52 (s, 2H), 2.98 (s, 3H); m/z = 356.2 [M + H]⁺. |
| 76 | 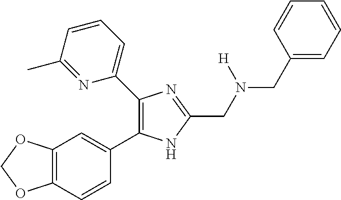 [SMILES: Cc1cccc(-c2nc(CNCc3ccccc3)[nH]c2-c2ccc3c(c2)OCO3)n1] | ¹H NMR (MeOD, 400 MHz) δ 8.23 (t, J = 8.0 Hz, 1H), 7.70-7.66 (m, 2H), 7.60-7.59 (m, 2H), 7.52-7.48 (m, 3H), 7.07-6.99 (m, 3H), 6.08 (s, 2H), 4.50 (s, 2H), 4.46 (s, 2H), 2.86 (s, 3H); m/z = 399.2 [M + H]⁺. |
| 77 | 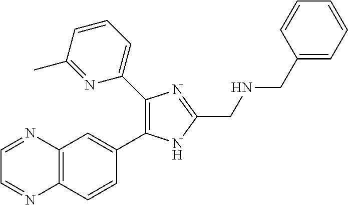 [SMILES: Cc1cccc(-c2nc(CNCc3ccccc3)[nH]c2-c2ccc3nccnc3c2)n1] | ¹H NMR (MeOD, 400 MHz) δ 9.00 (s, 2H), 8.35 (d, J = 1.6 Hz, 1H), 8.28-8.24 (d, J = 1.6 Hz, 1H), 8.04 (dd, J = 2.0, 8.8 Hz, 1H), 7.75 (dd, J = 8.0, 10.8 Hz, 2H), 7.65- 7.63 (m, 2H), 7.50-7.48 (m, 3H), 4.56 (s, 2H), 4.54 (s, 2H), 2.89 (s, 3H); m/z = 407.3 [M + H]⁺. |
| 78 | 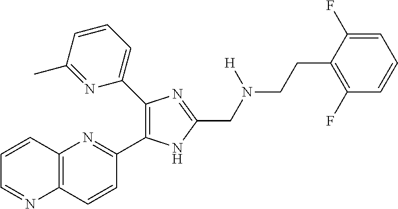 [SMILES: Cc1cccc(-c2nc(CNCCc3c(F)cccc3F)[nH]c2-c2ccc3ncccc3n2)n1] | ¹H NMR (MeOD, 400 MHz) δ 9.17 (dd, J = 1.6, 4.4 Hz, 1H), 8.91 (d, J = 8.4 Hz, 1H), 8.82 (d, J = 9.2 Hz, 1H), 8.71-8.49 (m, 3H), 8.06 (dd, J = 4.4, 8.8 Hz, 1H), 7.91 (d, J = 8.4 Hz, 1H), 7.46-7.36 (m, 1H), 7.08 (t, J = 8.0 Hz, 2H), 4.70 (s, 2H), 3.66-3.56 (m, 2H), 3.32-3.27 (m, 2H), 3.19 (s, 3H); m/z = 457.2 [M + H]⁺. |
| 79 | 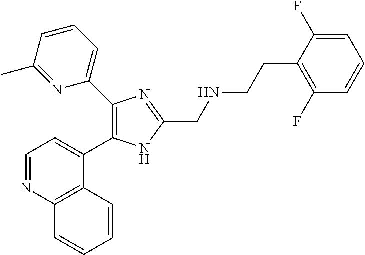 [SMILES: Cc1cccc(-c2nc(CNCCc3c(F)cccc3F)[nH]c2-c2ccnc3ccccc23)n1] | ¹H NMR (MeOD, 400 MHz) δ 9.16 (d, J = 5.6 Hz, 1H), 8.25 (d, J = 8.4 Hz, 2H), 8.14-8.07 (m, 2H), 7.94-7.86 (m, 1H), 7.85-7.79 (m, 1H), 7.55-7.46 (m, 1H), 7.32-7.25 (m, 2H), 6.94 (t, J = 8.0 Hz, 2H), 4.55 (s, 2H), 3.53-3.42 (m, 2H), 3.30-3.35 (m, 2H), 2.65 (s, 3H); m/z = 456.3 [M + H]⁺. |
| 80 | 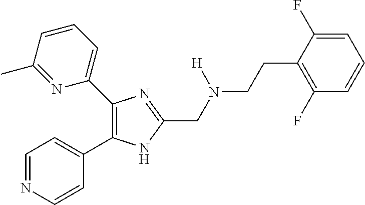 [SMILES: Cc1cccc(-c2nc(CNCCc3c(F)cccc3F)[nH]c2-c2ccncc2)n1] | ¹H NMR (MeOD, 400 MHz) δ 8.87 (d, J = 6.4 Hz, 2H), 8.46 (t, J = 8.0 Hz, 1H), 8.29 (d, J = 6.4 Hz, 2H), 8.02 (d, J = 8.0 Hz, 1H), 7.94 (d, J = 8.0 Hz, 1H), 7.43-7.32 (m, 1H), 7.11-6.96 (m, 2H), 4.63 (s, 2H), 3.57-3.50 (m, 2H), 3.30-3.22 (m, 2H), 2.91 (s, 3H); m/z = 406.3 [M + H]⁺. |
| 81 | 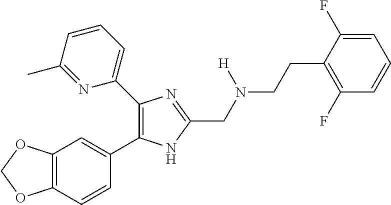 [SMILES: Cc1cccc(-c2nc(CNCCc3c(F)cccc3F)[nH]c2-c2ccc3c(c2)OCO3)n1] | ¹H NMR (MeOD, 400 MHz) δ 8.23 (t, J = 8.0 Hz, 1H), 7.68 (t, J = 9.2 Hz, 2H), 7.42-7.36 (m, 1H), 7.08-7.01 (m, 5H), 6.08 (s, 2H), 4.56 (s, 2H), 3.52-3.48 (m, 2H), 3.27- 3.23 (m, 2H), 2.86 (s, 3H); m/z = 449.2 [M + H]⁺. |
| 82 | 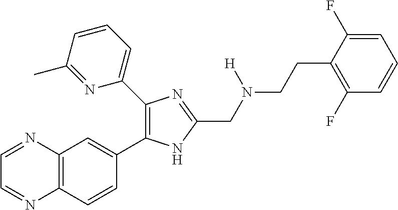 [SMILES: Cc1cccc(-c2nc(CNCCc3c(F)cccc3F)[nH]c2-c2ccc3nccnc3c2)n1] | ¹H NMR (MeOD, 400 MHz) δ 8.95 (s, 2H), 8.33 (s, 1H), 8.19 (d, J = 8.8 Hz, 1H), 8.04-8.00 (m, 2H), 7.57 (dd, J = 8.0, 16.4 Hz, 2H), 7.40-7.34 (m, 1H), 7.06-7.02 (m, 2H), 4.54 (s, 2H), 3.54-3.50 (m, 2H), 3.26-3.22 (m, 2H), 2.75 (s, 3H); m/z = 457.2 [M + H]⁺. |
| 83 | 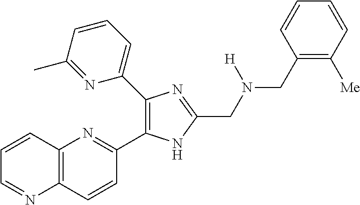 [SMILES: Cc1cccc(-c2nc(CNCc3ccccc3C)[nH]c2-c2ccc3ncccc3n2)n1] | ¹H NMR (MeOD, 400 MHz) δ 8.90 (d, J = 4.4 Hz, 1H), 8.42 (s, 1H), 8.33 (d, J = 9.2 Hz, 1H), 8.21 (d, J = 8.8 Hz, 1H), 7.85-7.76 (m, 3H), 7.35-7.14 (m, 2H), 7.15 (d, J = 2.6 Hz, 3H), 4.05 (s, 2H), 3.86 (s, 2H), 2.56 (s, 3H), 2.36 (s, 3H); m/z = 421.2 [M + H]⁺ |
| 84 | 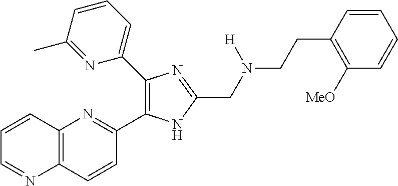 [SMILES: COc1ccccc1CCNCc1nc(-c2cccc(C)n2)c(-c2ccc3ncccc3n2)[nH]1] | ¹H NMR (MeOD, 400 MHz) δ 8.91-8.90 (m, 1H), 8.43 (d, J = 8.8 Hz, 1H), 8.32 (d, J = 8.8 Hz, 1H), 8.20 (d, J = 8.8 Hz, 1H), 7.84-7.74 (m, 3H), 7.27 (d, J = 7.0 Hz, 1H), 7.19-7.12 (m, 2H), 6.90-6.82 (m, 2H), 4.01 (s, 2H), 3.77 (s, 3H), 2.95-2.85 (m, 4H), 2.56 (s, 3H); m/z = 451.2 [M + H]⁺ |
| 85 | 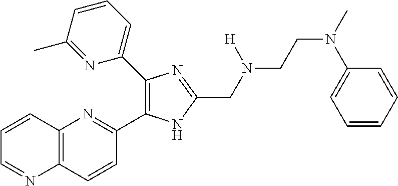 [SMILES: Cc1cccc(-c2nc(CNCCN(C)c3ccccc3)[nH]c2-c2ccc3ncccc3n2)n1] | ¹H NMR (MeOD, 400 MHz) δ 8.91-8.89 (m, 1H), 8.42 (d, J = 7.8 Hz, 1H), 8.32 (d, J = 9.6 Hz, 1H), 8.18 (d, J = 9.2 Hz, 1H), 7.80-7.73 (m, 3H), 7.27 (d, J = 6.6 Hz, 1H), 7.12 (dd, J = 7.6, 8.8 Hz, 2H), 6.78 (d, J = 7.8 Hz, 2H), 6.61 (t, J = 7.2 Hz, 1H), 4.02 (s, 2H), 3.49 (t, J = 7.0 Hz, 2H), 2.92 (s, 3H), 2.89 (t, J = 6.8 Hz, 2H), 2.55 (s, 3H); m/z = 450.2 [M + H]⁺ |
| 86 | 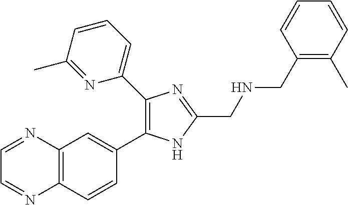 [SMILES: Cc1cccc(-c2nc(CNCc3ccccc3C)[nH]c2-c2ccc3nccnc3c2)n1] | ¹H NMR (MeOD, 400 MHz): δ 8.89 (q, J = 1.6 Hz, 2H), 8.30 (d, J = 1.6 Hz, 1H), 8.12-8.01 (m, 2H), 7.67 (t, J = 7.6 Hz, 1H), 7.40-7.32 (m, 2H), 7.22 (d, J = 8.0 Hz, 1H), 7.13-7.19 (m, 3H), 4.04 (s, 2H), 3.88 (s, 2H), 2.54 (s, 3H), 2.38 (s, 3H); m/z = 421.2 [M + H]⁺ |
| 87 | 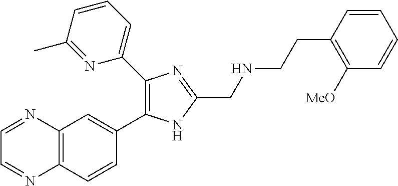 [SMILES: COc1ccccc1CCNCc1nc(-c2cccc(C)n2)c(-c2ccc3nccnc3c2)[nH]1] | ¹H NMR (MeOD, 400 MHz): δ 8.91-8.86 (m, 2H), 8.30 (d, J = 1.6 Hz, 1H), 8.12-8.01 (m, 2H), 7.66 (t, J = 7.6 Hz, 1H), 7.37 (d, J = 8.0 Hz, 1H), 7.24-7.14 (m, 3H), 6.94-6.84 (m, 2H), 3.99 (s, 2H), 3.80 (s, 3H), 2.86-2.96 (m, 4H), 2.54 (s, 3H); m/z = 451.3 [M + H]⁺ |
| 88 | 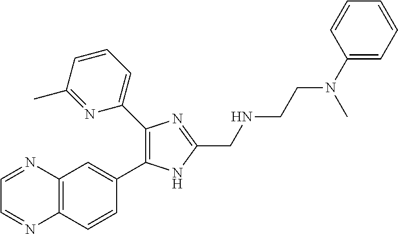 [SMILES: Cc1cccc(-c2nc(CNCCN(C)c3ccccc3)[nH]c2-c2ccc3nccnc3c2)n1] | ¹H NMR (MeOD, 400 MHz): δ 8.87-8.83 (m, 2H), 8.26 (d, J = 1.6 Hz, 1H), 8.08-8.03 (m, 1H), 8.01-7.96 (m, 1H), 7.62 (t, J = 8.0 Hz, 1H), 7.32 (d, J = 7.6 Hz, 1H), 7.19 (d, J = 7.6 Hz, 1H), 7.15-7.08 (m, 2H), 6.77 (d, J = 8.0 Hz, 2H), 6.61 (t, J = 7.2 Hz, 1H), 3.98 (s, 2H), 3.48 (t, J = 6.8 Hz, 2H), 2.92 (s, 3H), 2.88 (t, J = 6.8 Hz, 2H), 2.51 (s, 3H); m/z = 450.3 [M + H]⁺ |
| 89 | 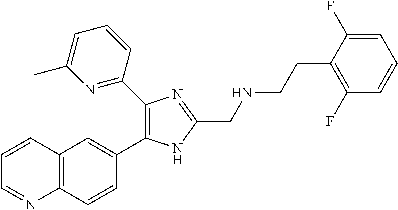 [SMILES: Cc1cccc(-c2nc(CNCCc3c(F)cccc3F)[nH]c2-c2ccc3ncccc3c2)n1] | ¹H NMR (MeOD, 400 MHz): δ 8.83 (dd, J = 4.0, 1.6 Hz, 1H), 8.33 (d, J = 8.4 Hz, 1H), 8.13 (d, J = 1.6 Hz, 1H), 8.00 (d, J = 8.8 Hz, 1H), 7.88 (dd, J = 9.2, 2.0 Hz, 1H), 7.62-7.52 (m, 2H), 7.28 (d, J = 8.0 Hz, 1H), 7.26-7.19 (m, 1H), 7.16 (d, J = 8.0 Hz, 1H), 6.96-6.89 (m, 2H), 3.98 (s, 2H), 2.98-2.86 (m, 5H), 2.51 (s, 3H); m/z = 456.2 [M + H]⁺ |
| 90 | 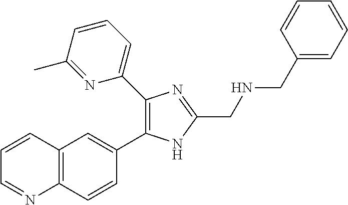 [SMILES: Cc1cccc(-c2nc(CNCc3ccccc3)[nH]c2-c2ccc3ncccc3c2)n1] | ¹H NMR (MeOD, 400 MHz): δ 8.87 (dd, J = 4.4, 1.6 Hz, 1H), 8.37 (d, J = 8.8 Hz, 1H), 8.17 (d, J = 1.6 Hz, 1H), 8.03 (d, J = 8.8 Hz, 1H), 7.94-7.89 (m, 1H), 7.66-7.55 (m, 2H), 7.43-7.39 (m, 2H), 7.39-7.25 (m, 5H), 7.20 (d, J = 7.6 Hz, 1H), 3.99 (s, 2H), 3.88 (s, 2H), 2.55 (s, 3H); m/z = 406.3 [M + H]⁺ |
| 91 | 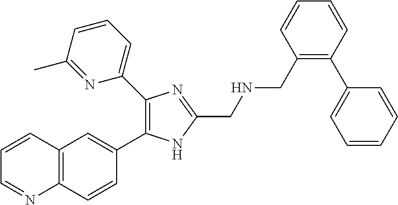 [SMILES: Cc1cccc(-c2nc(CNCc3ccccc3-c3ccccc3)[nH]c2-c2ccc3ncccc3c2)n1] | ¹H NMR (MeOD, 400 MHz): δ 8.89-8.84 (m, 1H), 8.37 (d, J = 7.6 Hz, 1H), 8.12 (d, J = 1.6 Hz, 1H), 8.06-8.00 (m, 1H), 7.91-7.85 (m, 1H), 7.66-7.55 (m, 3H), 7.43-7.26 (m, 8H), 7.25-7.17 (m, 3H), 3.89 (s, 2H), 3.83 (s, 2H), 2.55 (s, 3H); m/z = 482.3 [M + H]⁺ |

TABLE 4

| | | | MS |
|---|---|---|---|
| Cmpd | Structure and Name | ¹H NMR | (M + H) |
| 1.1 | 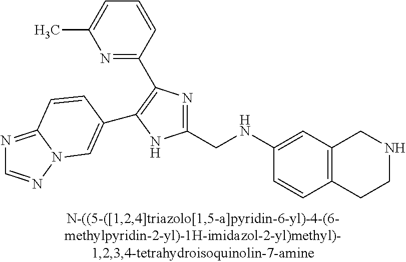 [SMILES: Cc1cccc(-c2nc(CNc3ccc4c(c3)CNCC4)[nH]c2-c2ccc3ncnn3c2)n1] | ¹H NMR (DMSO-d⁶) δ 9.51 (m, 3H), 8.65 (s, 1 H), 7.96 (d, J = 9.6 Hz, 1H), 7.87 (m, 2H), 7.70 (d, J = 8.0 Hz, 1H), 7.37 (d, J = 7.8 Hz, 1H), 6.97 (d, J = 8.8 Hz, 1H), 6.73 (d, J = 6.0 Hz, 2H), 4.75 (s, 2H), 4.13-4.10 (m, 2H), 3.25 (s, 2H), 2.83 (t, J = 6.0 Hz, 2H), 2.46 (s, 3 H). | 437.2 |
| 1.2 | 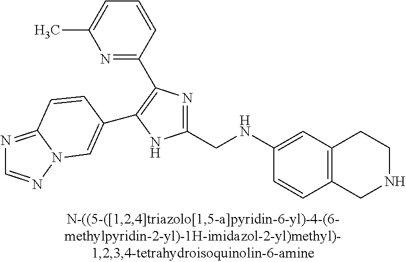 [SMILES: Cc1cccc(-c2nc(CNc3ccc4c(c3)CCNC4)[nH]c2-c2ccc3ncnn3c2)n1] | ¹H NMR (DMSO-d₆, 400 MHz) δ9.83 (s, 2H), 9.52 (s, 1H), 8.66 (s, 1H), 7.98-7.96 (m, 1H), 7.91-7.80 (m, 2H), 7.68 (d, J = 8.0 Hz, 1H), 7.37 (d, J = 8.0 Hz, 1H), 7.09 (t, J = 8.0 Hz, 1H), 6.76 (d, J = 8.0 Hz, 1H), 6.57 (d, J = 7.6 Hz, 1H), 4.84 (s, 2H), 4.21 (s, 2H), 3.27 (s, 2H), 2.94 (s, 2H), 2.46 (s, 3H) | 437.3 |
| 1.3 | 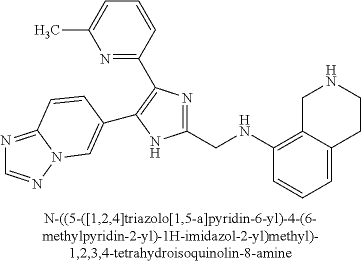 [SMILES: Cc1cccc(-c2nc(CNc3cccc4c3CNCC4)[nH]c2-c2ccc3ncnn3c2)n1] | ¹H NMR (DMSO-d₆, 400 MHz) δ 9.63 (s, 2H), 9.54 (s, 1H), 8.67 (s, 1H), 8.01-7.95 (m, 1H), 7.94-7.88 (m, 1H), 7.87-7.81 (m, 1H), 7.72 (d, J = 7.6 Hz, 1H), 7.36 (d, J = 7.6 Hz, 1H), 7.07 (t, J = 7.6 Hz, 1H), 6.84 (d, J = 8.0 Hz, 1H), 6.55 (d, J = 7.6 Hz, 1H), 4.87 (s, 2H), 4.14 (s, 2H), 3.37 (s, 2H), 2.93 (s, 2H), 2.47 (s, 3H) | 437.3 |
| 1.4 | 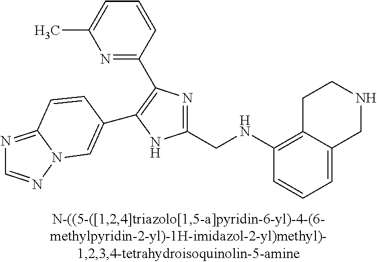 [SMILES: Cc1cccc(-c2nc(CNc3cccc4c3CCNC4)[nH]c2-c2ccc3ncnn3c2)n1] | ¹H NMR (DMSO-d₆, 400 MHz) δ 9.58 ( s, 2H), 9.53 (s, 1H), 8.67 (s, 1H), 7.99-7.95 (m, 1H), 7.92-7.83 (m, 2H), 7.72 (d, J = 8.0 Hz, 1H), 7.37 (d, 8.0 Hz, 1H), 6.97 (d, J = 8.0 Hz, 1H), 6.78-6.68 (m, 2H), 4.77 (s, 2H), 4.05 (s, 2H), 3.24 (s, 2H), 2.91 (s, 2H), 2.48 (s, 3H) | 437.3 |
| 1.5 | 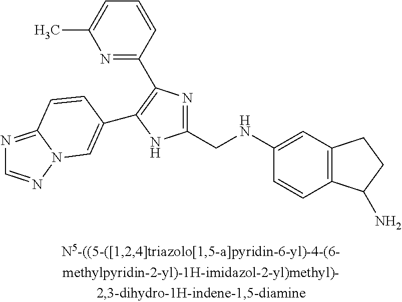 [SMILES: Cc1cccc(-c2nc(CNc3ccc4c(c3)CCC4N)[nH]c2-c2ccc3ncnn3c2)n1] | ¹H NMR (DMSO-d₆, 400 MHz) δ 9.54 (s, 1H), 8.49 (s, 1H), 7.98 (dd, J = 1.6, 9.4 Hz, 1H), 7.82 (d, J = 9.2 Hz, 1H), 7.70 (t, J = 7.8 Hz, 1H), 7.15 (d, J = 7.4 Hz, 1H), 7.05 (d, J = 8.6 Hz, 1H), 6.57-6.55 (m, 2H), 5.91 (t, J = 5.8 Hz, 1H), 4.33 (d, J = 5.8 Hz, 2H), 4.07 (t, J = 7.2 Hz, 1H), 2.75 (ddd, J = 3.2, 8.4, 15.6 Hz, 1H), 2.60-2.55 (m, 1H), 2.46 (s, 3H), 2.21-2.30 (m, 1H), 1.55-1.46 (m, 1H) | 459.2 |
| 1.6 | 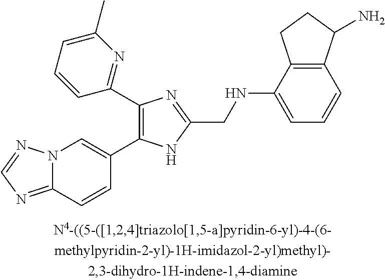 [SMILES: Cc1cccc(-c2nc(CNc3cccc4c3CCC4N)[nH]c2-c2ccc3ncnn3c2)n1] | ¹H NMR (DMSO-d₆, 400 MHz) δ 9.50 (s, 1H), 8.63 (s, 1H), 8.45 (s, 3H), 7.97 (d, J = 9.8 Hz, 1H), 7.87- 7.81 (m, 2H), 7.66 (d, J = 7.6 Hz, 1H), 7.36 (d, J = 7.6 Hz, 1H), 7.14- 7.10 (m, 1H), 6.96 (d, J = 7.6 Hz, 1H), 6.80 (d, J = 7.6 Hz, 1H), 4.82 (s, 2H), 4.62 (s, 1H), 3.06-3.00 (m, 1H), 2.86-2.78 (m, 1H), 2.49 (s, 3H), 2.03-1.98 (m, 1H) | 437.3 |
| 1.7 | 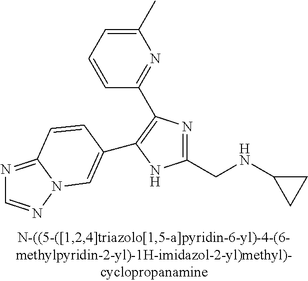 [SMILES: Cc1cccc(-c2nc(CNC3CC3)[nH]c2-c2ccc3ncnn3c2)n1] | ¹H NMR (DMSO-d₆, 400 MHz) δ 9.54 (s, 1H), 8.65 (s, 1H), 8.10-7.91 (m, 3H), 7.78-7.63 (m, 1H), 7.50 (d, J = 7.2 Hz, 1H), 4.48 (s, 2H), 3.00- 2.87 (m, 1H), 2.64 (s, 3H), 1.00 (s, 2H), 0.80 (d, J = 5.2 Hz, 2H) | 346.1 |
| 1.8 | 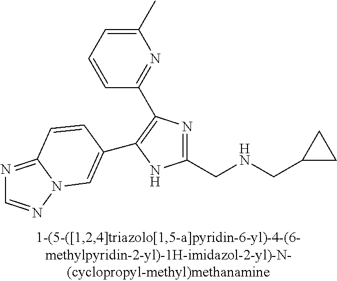 [SMILES: Cc1cccc(-c2nc(CNCC3CC3)[nH]c2-c2ccc3ncnn3c2)n1] | ¹H NMR (DMSO-d₆, 400 MHz) δ10.14 (s, 2H), 9.52 (s, 1H), 8.77 (s, 1H), 8.15-8.09 (m, 3H), 8.03 (d, J = 9.2 Hz, 3H), 8.00-7.95 (m, 2H), 7.76 (d, J = 8.0 Hz, 1H), 7.61 (d, J = 7.6 Hz, 1H), 4.49 (s, 2H), 3.05 (s, 2H), 2.68 (s, 3H), 1.20 (s, 1H), 0.58 (d, J = 7.6 Hz, 2H), 0.46 (d, J = 4.4 Hz, 2H) | 360.3 |
| 1.9 | 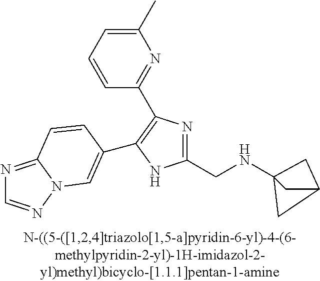 [SMILES: Cc1cccc(-c2nc(CNC34CC(C3)C4)[nH]c2-c2ccc3ncnn3c2)n1] | ¹¹H NMR (DMSO-d₆, 400 MHz) δ12.48 (s, 1H), 9.57 (s, 1H), 8.50 (s, 1H), 8.01 (s, 1H), 7.90-7.64 (m, 2H), 7.42 (s, 1H), 7.17-7.07 (m, 1H), 3.81 (s, 2H), 2.48-2.30 (m, 3H), 1.75- 1.68 (m, 6H) | 372.3 |
| 1.10 | 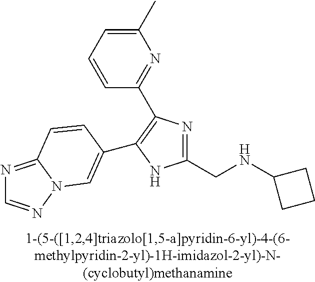 [SMILES: Cc1cccc(-c2nc(CNC3CCC3)[nH]c2-c2ccc3ncnn3c2)n1] | ¹H NMR (DMSO-d₆, 400 MHz) δ 9.56 (s, 1H), 8.49 (s, 1H), 8.00 (d, J = 9.2 Hz, 1H), 7.82 (d, J = 9.2 Hz, 1H), 7.69 (t, J = 7.6 Hz, 1H), 7.51 (d, J = 14.4 Hz, 1H), 7.13 (d, J = 7.2 Hz, 1H), 3.72 (s, 2H), 3.31-3.13 (m, 2H), 2.46 (s, 3H), 2.09-2.02 (m, 1H), 2.13-2.01 (m, 1H), 1.74-1.48 (m, 4H) | 360.2 |
| 1.11 | 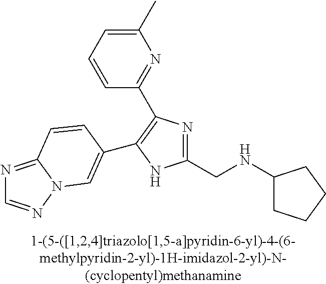 [SMILES: Cc1cccc(-c2nc(CNC3CCCC3)[nH]c2-c2ccc3ncnn3c2)n1] | ¹H NMR (DMSO-d₆, 400 MHz) δ 9.56 (br s, 1H), 8.50 (s, 1H), 7.99 (s, 1H), 7.83 (s, 1H), 7.73 (s, 1H), 7.52 (s, 1H), 7.15 (s, 1H), 3.80 (s, 2H), 3.08 (s, 1H), 2.46 (s, 3H), 1.83-1.29 (m, 10H) | 374.2 |
| 1.12 | 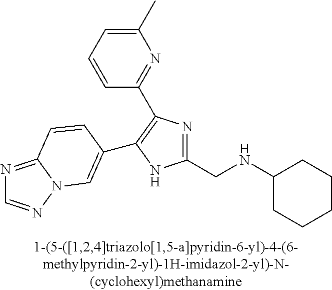 [SMILES: Cc1cccc(-c2nc(CNC3CCCCC3)[nH]c2-c2ccc3ncnn3c2)n1] | ¹H NMR (DMSO-d₆, 400 MHz) δ 9.55 (s, 1H), 9.65-9.49 (m, 1H), 8.49 (s, 1H), 7.99 (d, J = 6.8 Hz, 1H), 7.83 (s, 1H), 7.70 (s, 1H), 7.52 (s, 1H), 7.15 (s, 1H), 3.83 (s, 2H), 2.46 (s, 3H), 1.85 (s, 2H), 1.67 (s, 2H), 1.55 (s, 1H), 1.31-0.98 (m, 6H) | 388.3 |
| 1.13 | 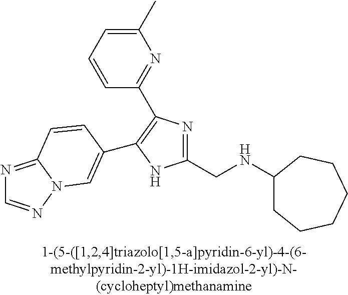 [SMILES: Cc1cccc(-c2nc(CNC3CCCCCC3)[nH]c2-c2ccc3ncnn3c2)n1] | ¹H NMR (DMSO-d₆, 400 MHz) δ 9.55 (br s, 1H), 8.49 (s, 1H), 7.99 (d, J = 8.8 Hz, 1H), 7.82 (d, J = 9.2 Hz, 1H), 7.70 (t, J = 7.6 Hz, 1H), 7.51 (s, 1H), 7.14 (d, J = 7.2 Hz, 1H), 3.79 (s, 2H), 2.75-2.58 (m, 1H), 2.47 (s, 3H), 1.81 (s, 2H), 1.62 (s, 2H), 1.49 (s, 4H), 1.37 (s, 4H) | 402.3 |
| 1.14 | 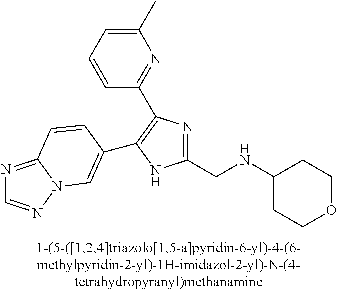 [SMILES: Cc1cccc(-c2nc(CNC3CCOCC3)[nH]c2-c2ccc3ncnn3c2)n1] | ¹H NMR (DMSO-d₆, 400 MHz) δ 9.55 (s, 1H), 8.50 (s, 1H), 7.99 (d, J = 9.2 Hz, 1H), 7.83 (d, J = 9.0 Hz, 1H), 7.71 (t, J = 7.8 Hz, 1H), 7.16 (s, 1H), 3.85 (s, 3H), 3.83 (d, J = 3.6 Hz, 1H), 3.42-3.19 (m, 4H), 2.75- 2.63 (m, 1H), 2.46 (s, 3H), 1.81 (d, J = 12.0 Hz, 2H), 1.40-1.19 (m, 2H) | 390.2 |
| 1.15 | 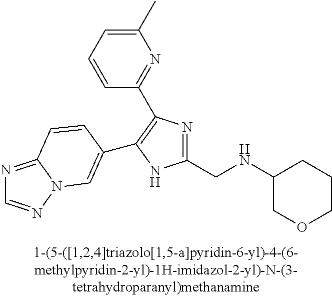 [SMILES: Cc1cccc(-c2nc(CNC3CCCOC3)[nH]c2-c2ccc3ncnn3c2)n1] | ¹H NMR (DMSO-d₆, 400 MHz) δ 9.54 (s, 1H), 8.49 (s, 1H), 7.98 (d, J = 9.2 Hz, 1H), 7.82 (d, J = 9.2 Hz, 1H), 7.70 (t, J = 8.0 Hz, 1H), 7.15 (s, 1H), 3.87-3.84 (m, 1H), 3.83 (d, J = 1.6 Hz, 2H), 3.71-3.64 (m, 1H), 3.30-3.23 (m, 3H), 3.04 (dd, J = 8.8, 10.8 Hz, 1H), 2.62-2.54 (m, 2H), 2.46 (s, 3H), 1.94 (d, J = 9.2 Hz, 1H), 1.68-1.55 (m, 1H), 1.51-1.38 (m, 1H), 1.34-1.23 (m, 1H) | 390.3 |
| 1.16 | 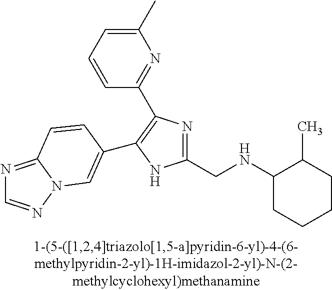 [SMILES: Cc1cccc(-c2nc(CNC3CCCCC3C)[nH]c2-c2ccc3ncnn3c2)n1] | ¹H NMR (DMSO-d₆, 400 MHz) δ 9.53 (s, 2H), 8.82 (s, 1H), 8.13-7.96 (m, 3H), 7.75 (d, J = 8.0 Hz, 2H), 7.58 (d, J = 8.0 Hz, 3H), 4.67-4.43 (m, 2H), 3.09-2.94 (m, 1H), 2.64 (s, 3H), 2.22 (d, J = 10.2 Hz, 1H), 1.75 (t, J = 13.2 Hz, 3H), 1.61-1.47 (m, 2H), 1.35 (s, 1H), 1.30-1.14 (m, 2H), 1.10 (d, J = 6.4 Hz, 3H), 1.04 (d, J = 7.2 Hz, 1H), 1.06-1.02 (m, 1H) | 402.3 |
| 1.17 | 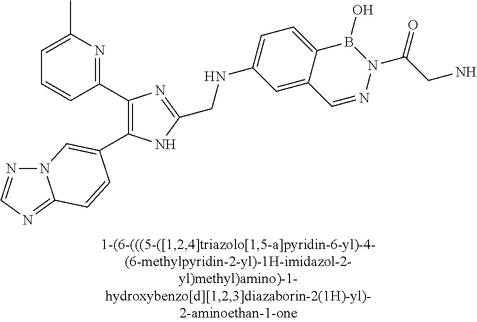 [SMILES: Cc1cccc(-c2nc(CNc3ccc4c(c3)C=NN(C(=O)CN)B4O)[nH]c2-c2ccc3ncnn3c2)n1] | | 507.2 |
| 1.18 | 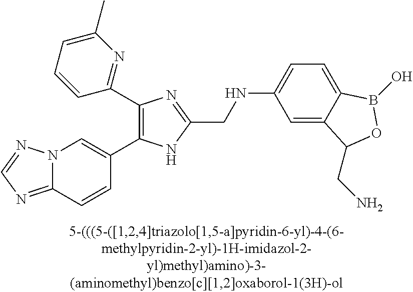 [SMILES: Cc1cccc(-c2nc(CNc3ccc4c(c3)C(CN)OB4O)[nH]c2-c2ccc3ncnn3c2)n1] | | 467.2 |
| 1.19 | 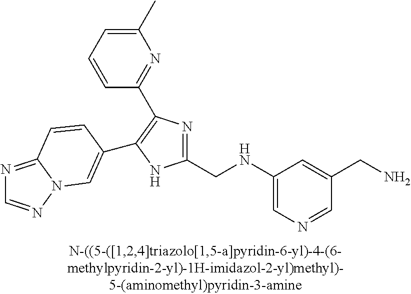 [SMILES: Cc1cccc(-c2nc(CNc3cncc(CN)c3)[nH]c2-c2ccc3ncnn3c2)n1] | ¹H NMR (DMSO-d₆, 400 MHz) δ 9.48 (s, 1H), 8.88 (br s, 3H), 8.64 (s, 1H), 8.42 (d, J = 2.4 Hz, 1H), 8.31 (s, 1H), 8.21 (s, 1H), 8.00-7.83 (m, 3H), 7.64 (d, J = 8.0 Hz, 1H), 7.43 (d, J = 8.0 Hz, 1H), 4.90 (s, 2H), 4.18 (d, J = 5.6 Hz, 1H), 4.21-4.14 (m, 1H), 4.23-4.13 (m, 1H), 2.56 (s, 3H) | 412.1 |
| 1.20 | 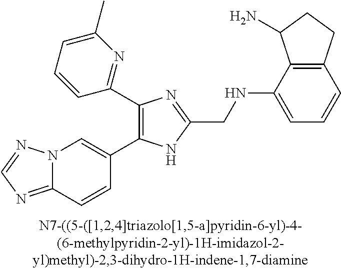 [SMILES: Cc1cccc(-c2nc(CNc3cccc4c3C(N)CC4)[nH]c2-c2ccc3ncnn3c2)n1] | ¹H NMR (DMSO-d₆, 400 MHz) δ 9.42 (s, 1H), 8.59 (s, 1H), 8.01 (s, 3H), 7.92 (d, J = 9.4 Hz, 1H), 7.80- 7.76 (m, 1H), 7.45 (d, J = 8.0 Hz, 1H), 7.27 (s, 1H), 7.31 (d, J = 7.6 Hz, 1H), 7.18 (t, J = 7.8 Hz, 1H), 6.66 (d, J = 7.6 Hz, 1H), 6.57 (d, J = 8.0 Hz, 1H), 4.89 (s, 1H), 4.66 (s, 2H), 3.12-3.16 (m, 1H), 2.78-2.83 (m, 1H), 2.53 (s, 3H), 2.42-2.32 (m, 1H), 2.12-2.06 (m, 1H) | 459.2 (M + Na) |
| 1.21 | 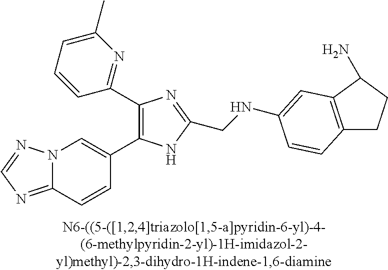 [SMILES: Cc1cccc(-c2nc(CNc3ccc4c(c3)C(N)CC4)[nH]c2-c2ccc3ncnn3c2)n1] | ¹H NMR (DMSO-d₆, 400 MHz) δ 9.54 (s, 1H), 8.49 (s, 1H), 7.99 (dd, J = 1.6, 9.4 Hz, 1H), 7.82 (d, J = 9.4 Hz, 1H), 7.70 (t, J = 7.8 Hz, 1H), 7.59-7.44 (m, 1H), 7.14 (d, J = 7.8 Hz, 1H), 6.92 (d, J = 8.0 Hz, 1H), 6.79 (s, 1H), 6.57 (dd, J = 1.8, 8.0 Hz, 1H), 5.89 (t, J = 5.8 Hz, 1H), 4.35 (d, J = 5.8 Hz, 2H), 4.12 (t, J = 7.4 Hz, 1H), 2.74-2.66 (m, 1H), 2.59-2.53 (m, 2H), 2.46 (s, 3H), 2.33-2.24 (m, 1H), 1.52-1.58 (m, 1H) | 459.1 (M + Na) |
| 1.22 | 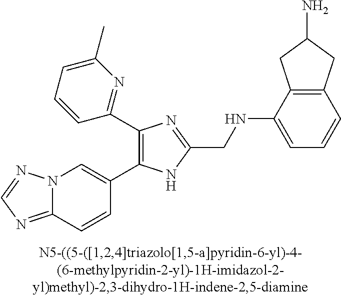 [SMILES: Cc1cccc(-c2nc(CNc3cccc4c3CC(N)C4)[nH]c2-c2ccc3ncnn3c2)n1] | ¹H NMR (DMSO-d₆, 400 MHz) δ 9.45 (s, 1H), 8.57 (s, 1H), 8.10 (s, 3H), 7.95-7.81 (m, 2H), 7.77 (t, J = 7.6 Hz, 1H), 7.47 (d, J = 8.0 Hz, 1H), 7.28 (d, J = 7.6 Hz, 1H), 7.04 (t, J = 7.6 Hz, 1H), 6.57 (dd, J = 7.6, 15.4 Hz, 2H), 4.58 (s, 3H), 4.01 (s, 2H), 3.29-3.13 (m, 2H), 2.92-2.76 (m, 2H) | 437.2 |
| 1.23 | 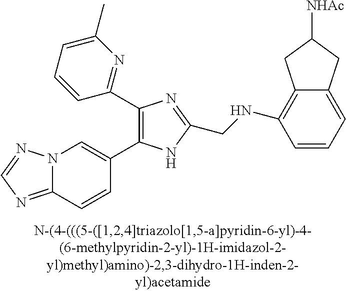 [SMILES: CC(=O)NC1Cc2cccc(NCc3nc(-c4cccc(C)n4)c(-c4ccc5ncnn5c4)[nH]3)c2C1] | ¹H NMR (MeOD, 400 MHz) δ 9.20 (s, 1H), 8.70-8.36 (m, 4H), 8.10-7.93 (m, 2H), 7.85-7.71 (m, 1H), 7.85- 7.71 (m, 1H), 7.38 (d, J = 7.6 Hz, 1H), 7.29 (br s, 1H), 5.38 (br d, J = 6.8 Hz, 1H), 5.32 (s, 2H), 3.98 (dd, J = 7.8, 15.6 Hz, 2H), 3.61-3.45 (m, 2H), 2.70 (s, 3H) | 479.3 |
| 1.24 | 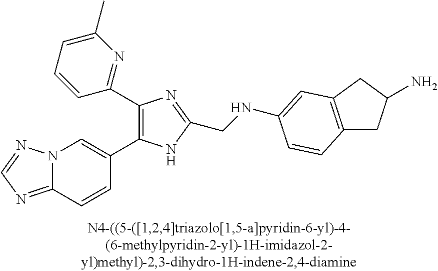 [SMILES: Cc1cccc(-c2nc(CNc3ccc4c(c3)CC(N)C4)[nH]c2-c2ccc3ncnn3c2)n1] | ¹H NMR (DMSO-d₆, 400 MHz) δ 9.41 (s, 1H), 8.61 (s, 1H), 8.06 (d, J = 3.2 Hz, 2H), 7.95 (dd, J = 0.8, 9.6 Hz, 1H), 7.83-7.78 (m, 2H), 7.46 (d, J = 8.0 Hz, 1H), 7.35 (d, J = 7.6 Hz, 1H), 7.03 (d, J = 8.0 Hz, 1H), 6.65 (s, 1H), 6.59 (dd, J = 2.4, 8.0 Hz, 1H), 4.62 (s, 2H), 3.94-3.92 (m, 1H), 3.19-3.11 (m, 2H), 2.88-2.73 (m, 2H), 2.52 (s, 3H) | 437.2 |
| 1.25 | 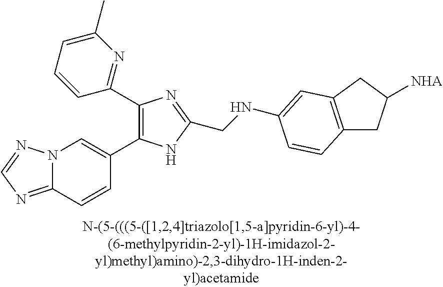 | ¹H NMR (DMSO-d₆, 400 MHz) δ9.51 (br s, 1H), 8.52 (br s, 1H), 8.06 (br d, J = 6.0 Hz, 1H), 7.95 (d, J = 8.4 Hz, 1H), 7.89-7.79 (m, 1H), 7.79-7.67 (m, 1H), 7.51 (s, 1H), 7.20 (s, 2H), 7.08 (s, 1H), 6.99-6.88 (m, 2H), 6.70-6.49 (m, 2H), 4.36 (s, 3H), 3.11-2.93 (m, 2H), 2.68-2.56 (m, 3H), 2.46 (s, 3H), 1.77 (s, 3H) | 479.3 |
| 1.26 | 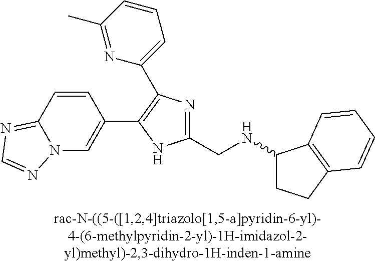 [SMILES: Cc1cccc(-c2nc(CNC3CCc4ccccc43)[nH]c2-c2ccc3ncnn3c2)n1] | 1H NMR (DMSO-d6, 400 MHz) δ 9.19 (s, 1H), 8.43 (s, 1H), 7.41 (s, 2H), 7.89 (d, J = 10.8 Hz, 1H), 7.78 (d, J = 9.2 Hz, 1H), 7.65 (t, J = 8.0 Hz, 1H), 7.44-7.41 (m, 1H), 7.21 (d, J = 2.0 Hz, 1H), 7.25-7.15 (m, 4H), 4.35 (t, J = 6.0 Hz, 1H), 4.05 (d, J = 2.0 Hz, 2H), 3.11-3.02 (m, 1H), 2.88-2.80 (m, 1H), 2.51 (s, 3H), 2.46-2.36 (m, 1H), 2.02-1.39 (m, 1H) | 422.2 |
| 1.27 | 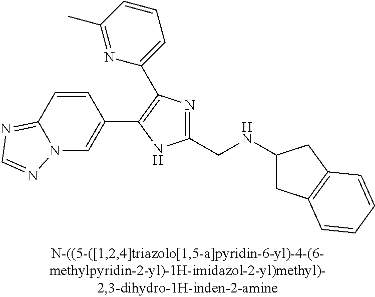 [SMILES: Cc1cccc(-c2nc(CNC3Cc4ccccc4C3)[nH]c2-c2ccc3ncnn3c2)n1] | 1H NMR (DMS0-d6, 400 MHz) δ 9.55 (s, 1H), 8.49 (s, 1H), 7.91 (d, J = 12.0 Hz, 1H), 7.82 (d, J = 8.8 Hz, 1H), 7.69 (t, J = 7.2 Hz, 1H), 7.65 (t, J = 8.0 Hz, 1H), 7.20-7.09 (m, 5H), 3.88 (s, 2H), 3.60 (t, J = 6.8 Hz, 1H), 3.10-3.03 (m, 2H), 2.75-2.68 (m, 2H) | 422.2 |
| 1.30 | 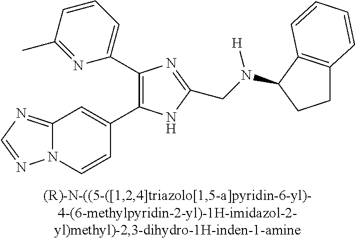 [SMILES: Cc1cccc(-c2nc(CN[C@@H]3CCc4ccccc43)[nH]c2-c2ccn3ncnc3c2)n1] | ¹H NMR (DMSO-d₆, 400 MHz) δ 9.56 (s, 1H), 8.50 (s, 1H), 8.01 (d, J = 8.0 Hz, 1H), 7.83 (d, J = 12.0 Hz, 1H), 7.72 (t, J = 7.2 Hz, 1H), 7.48- 7.43 (m, 2H), 7.24-7.17 (m, 4H), 4.22 (t, J = 7.2 Hz, 1H), 3.91 (s, 2H), 3.01-2.93 (m, 1H), 2.77-2.73 (m, 1H), 2.52 (s, 3H), 2.33-2.29 (m, 1H), 1.82-1.79 (m, 1H) | 422.1 |
| 1.31 | 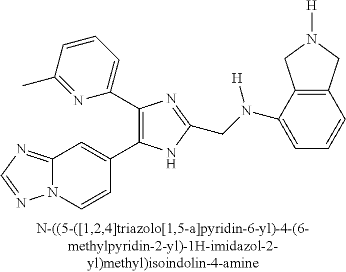 [SMILES: Cc1cccc(-c2nc(CNc3cccc4c3CNC4)[nH]c2-c2ccn3ncnc3c2)n1] | ¹H NMR (DMSO-d₆, 400 MHz) δ 9.43 (d, J = 1.0 Hz, 1H), 8.58 (s, 1H), 7.94-7.92 (m, 1H), 7.91-7.90 (m, 2H), 7.47 (d, J = 7.8 Hz, 1H), 7.31 (d, J = 7.8 Hz, 1H), 7.19 (t, J = 7.8 Hz, 1H), 6.68 (dd, J = 5.2, 7.6 Hz, 2H), 4.63 (s, 2H), 4.47 (t, J = 5.2 Hz, 2H), 4.42 (br t, J = 5.2 Hz, 2H), 2.53 (s, 3H) | 423.1 |
| 1.32 | 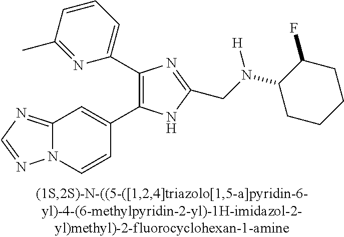 | ¹H NMR (DMSO-d₆, 400 MHz) δ 9.53 (s, 1H), 8.51 (s, 1H), 7.99 (d, J = 9.2 Hz, 1H), 7.84 (d, J = 9.2 Hz, 1H), 7.71 (t, J = 8.0 Hz, 1H), 7.44- 7.31 (m, 1H), 7.22-7.12 (m, 1H), 4.41-4.29 (m, 1H), 3.86 (d, J = 4.0 Hz, 2H), 2.68-2.59 (m, 1H), 2.56 (s, 3H), 2.03-1.95 (m, 3H), 1.69-1.55 (m, 2H), 1.18-1.13 (m, 4H) | 406.2 |
| 1.33 | 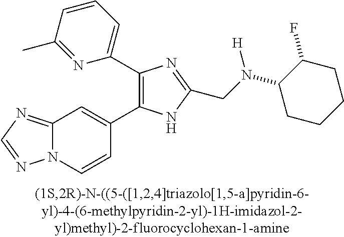 | ¹H NMR (MeOD, 400 MHz) δ 9.20 (s, 1H), 8.56 (s, 1H), 8.17 (t, J = 8.0 Hz, 1H), 7.97-7.81 (m, 2H), 7.68 (t, J = 8.6 Hz, 2H), 5.41-5.17 (m, 1H), 4.67-4.46 (m, 2H), 3.76-3.48 (m, 1H), 2.84 (s, 3H), 2.30-2.06 (m, 2H), 1.99-1.57 (m, 5H), 1.56-1.41 (m, 1H) | 406.1 |
| 1.34 | 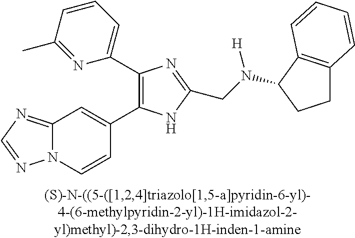 [SMILES: Cc1cccc(-c2nc(CN[C@H]3CCc4ccccc43)[nH]c2-c2ccn3ncnc3c2)n1] | | 422.1 |
| 1.35 | 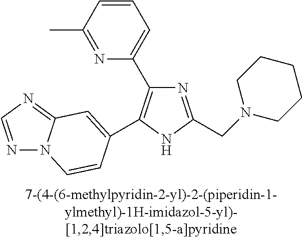 [SMILES: Cc1cccc(-c2nc(CN3CCCCC3)[nH]c2-c2ccn3ncnc3c2)n1] | | 374.2 |
| 1.36 | 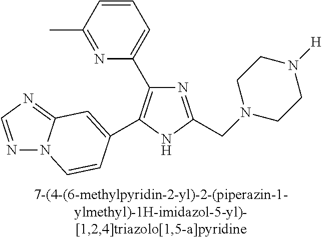 [SMILES: Cc1cccc(-c2nc(CN3CCNCC3)[nH]c2-c2ccn3ncnc3c2)n1] | | 375.2 |
| 1.37 | 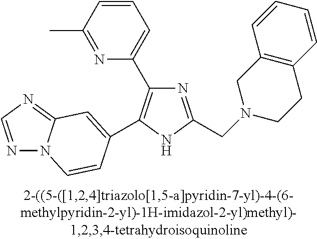 [SMILES: Cc1cccc(-c2nc(CN3CCc4ccccc4C3)[nH]c2-c2ccn3ncnc3c2)n1] | | 422.2 |
| 1.38 | 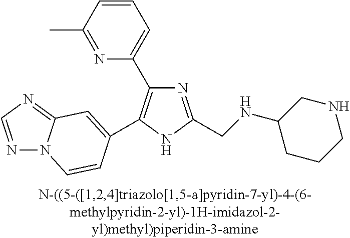 [SMILES: Cc1cccc(-c2nc(CNC3CCCNC3)[nH]c2-c2ccn3ncnc3c2)n1] | | 389.2 |
| 1.39 | 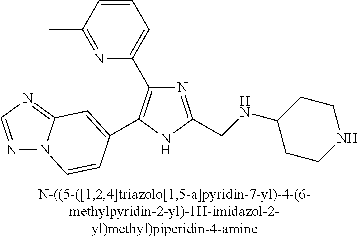 [SMILES: Cc1cccc(-c2nc(CNC3CCNCC3)[nH]c2-c2ccn3ncnc3c2)n1] | | 389.2 |
| 1.40 | 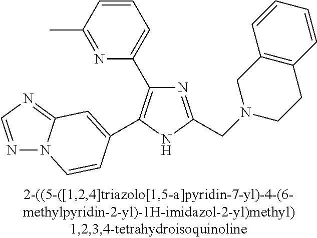 [SMILES: Cc1cccc(-c2nc(CN3CCc4ccccc4C3)[nH]c2-c2ccn3ncnc3c2)n1] | | 422.2 |
| 1.41 | 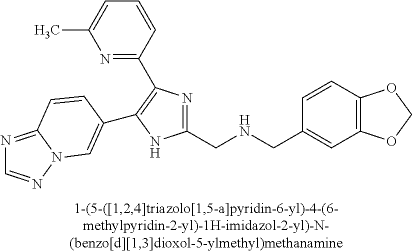 [SMILES: Cc1cccc(-c2nc(CNCc3ccc4c(c3)OCO4)[nH]c2-c2ccc3ncnn3c2)n1] | ¹H NMR (MeOD, 400 MHz) δ 9.20 (s, 1H), 8.53 (s, 1H), 8.10 (t, J = 8.0 Hz, 1H), 7.94-7.84 (m, 2H), 7.68- 7.59 (m, 2H), 7.06-7.03 (m, 2H), 6.91-6.87 (m, 1H), 6.00 (s, 2H), 4.46 (s, 2H), 4.39 (s, 2H), 2.79 (s, 3H) | 440.1 |
| 1.42 | 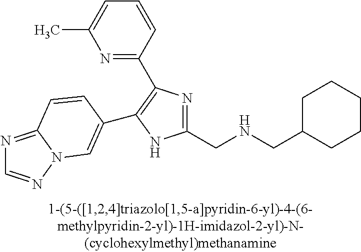 [SMILES: Cc1cccc(-c2nc(CNCC3CCCCC3)[nH]c2-c2ccc3ncnn3c2)n1] | ¹H NMR (MeOD, 400 MHz) δ 9.20 (s, 1H), 8.53 (s, 1H), 8.10 (t, J = 8.0 Hz, 1H), 7.93-7.89 (m, 1H), 7.88- 7.84 (m, 1H), 7.62 (dd, J = 8.0, 19.3 Hz, 2H), 4.47 (s, 2H), 3.11 (d, J = 6.8 Hz, 2H), 2.79 (s, 3H), 1.92-1.77 (m, 5H), 1.73 (d, J = 12.0 Hz, 1H), 1.41-1.21 (m, 3H), 1.16-1.02 (m, 2H) | 402.1 |
| 1.43 | 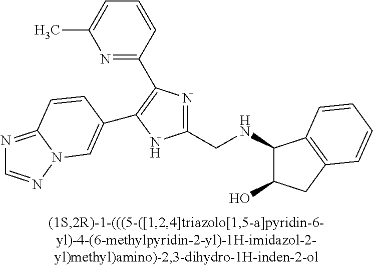 [SMILES: Cc1cccc(-c2nc(CN[C@H]3c4ccccc4C[C@H]3O)[nH]c2-c2ccc3ncnn3c2)n1] | ¹H NMR (MeOD, 400 MHz) δ 9.24- 9.14 (m, 1H), 8.55 (s, 1H), 8.15 (t, J = 8.0 Hz, 1H), 7.96-7.90 (m, 1H), 7.88-7.84 (m, 1H), 7.71-7.62 (m, 3H), 7.45-7.32 (m, 3H), 4.95 (dd, J = 4.8, 6.1 Hz, 2H), 4.65 (d, J = 1.2 Hz, 2H), 3.35 (d, J = 6.0 Hz, 1H), 3.30 (s, 1H), 3.18 (d, J = 4.8 Hz, 1H), 3.14 (d, J = 4.8 Hz, 1H), 2.82 (s, 3H) | 438.1 |
| 1.44 | 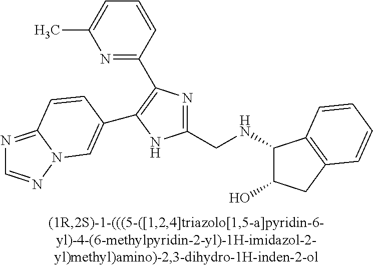 [SMILES: Cc1cccc(-c2nc(CN[C@@H]3c4ccccc4C[C@@H]3O)[nH]c2-c2ccc3ncnn3c2)n1] | ¹H NMR (MeOD, 400 MHz) δ 9.19 (s, 1H), 8.54 (s, 1H), 8.15 (t, J = 8.0 Hz, 1H), 7.96-7.81 (m, 2H), 7.71- 7.61 (m, 3H), 7.47-7.33 (m, 3H), 4.95-4.89 (s, 2H), 4.65 (s, 2H), 3.35 (d, J = 6.4 Hz, 1H), 3.16 (dd, J = 4.8, 16.4 Hz, 1H), 2.82 (s, 3H) | 438.1 |
| 1.45 | 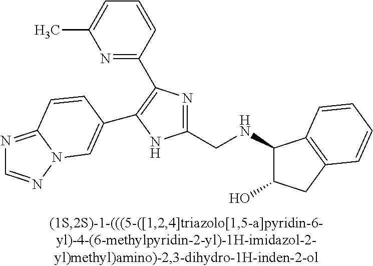 [SMILES: Cc1cccc(-c2nc(CN[C@H]3c4ccccc4C[C@@H]3O)[nH]c2-c2ccc3ncnn3c2)n1] | ¹H NMR (MeOD. 400 MHz) δ 9.21 (s, 1H), 8.54 (s, 1H), 8.09 (t, J = 8.0 Hz, 1H), 7.92-7.84 (m, 2H), 7.67- 7.64 (m, 2H), 7.59 (d, J = 7.8 Hz, 1H), 7.45-7.36 (m, 3H), 4.87-4.81 (m, 1H), 4.74 (q, J = 14.8 Hz, 2H), 3.57-3.48 (m, 1H), 3.07-2.96 (m, 1H), 2.78 (s, 3H) | 438.1 |
| 1.46 | 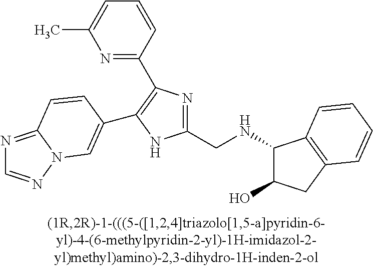 [SMILES: Cc1cccc(-c2nc(CN[C@@H]3c4ccccc4C[C@H]3O)[nH]c2-c2ccc3ncnn3c2)n1] | ¹H NMR (MeOD, 400 MHz) δ 9.25- 9.18 (m, 1H), 8.44 (s, 1H), 7.91 (dd, J = 1.6, 9.2 Hz, 1H), 7.78 (dd, J = 0.8, 9.2 Hz, 1H), 7.68 (t, J = 1.6 Hz, 1H), 7.46-7.38 (m, 2H), 7.25-7.22 (m, 3H), 7.19 (d, J = 7.6 Hz, 1H), 4.43 (td, J = 5.4, 6.4 Hz, 1H), 4.20 (s, 2H), 4.16 (d, J = 4.6 Hz, 1H), 3.36 (d, J = 6.8 Hz, 1H), 3.32-3.30 (m, 1H), 2.81 (dd, J = 5.6, 15.8 Hz, 1H), 2.53 (s, 3H) | 438.2 |
| 1.47 | 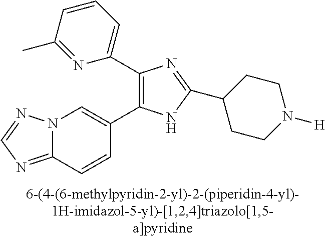 [SMILES: Cc1cccc(-c2nc(C3CCNCC3)[nH]c2-c2ccc3ncnn3c2)n1] | ¹H NMR (CDCl₃, 400 MHz) δ 9.44 (s, 1H), 8.94 (s, 1H), 8.65 (d, J = 6.0 Hz, 1H), 8.60 (s, 1H), 7.94 (d, J = 9.2 Hz, 1H), 7.87-7.80 (m, 3H), 7.52-7.47 (m, 1H), 7.36 (d, J = 7.6 Hz, 1H), 3.86 (s, 1H), 3.46 (d, J = 12.4 Hz, 2H), 3.41-3.33 (m, 1H), 3.17-3.04 (m, 2H), 2.56-2.53 (m, 3H), 2.27 (d, J = 12.4 Hz, 2H), 2.11-2.00 (m, 2H) | 360.0 |
| 1.48 | 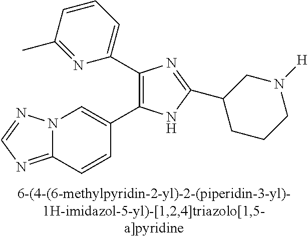 [SMILES: Cc1cccc(-c2nc(C3CCCNC3)[nH]c2-c2ccc3ncnn3c2)n1] | ¹H NMR (MeOD, 400 MHz) δ 9.20- 9.18 (m, 1H), 8.54 (s, 1H), 8.11-8.06 (m, 1H), 7.94-7.91 (m, 1H), 7.85- 7.82 (m, 1H), 7.61 (t, J = 8.4 Hz, 2H), 4.00 (s, 1H), 3.70-3.49 (m, 3H), 3.40-3.34 (m, 1H), 3.27-3.18 (m, 1H), 2.80 (s, 3H), 2.27-2.32 (m, 1H), 2.08-2.00 (m, 2H), 2.00-1.83 (m, 1H) | 360.0 |
| 1.49 | 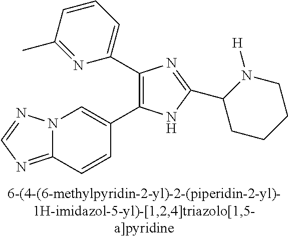 [SMILES: Cc1cccc(-c2nc(C3CCCCN3)[nH]c2-c2ccc3ncnn3c2)n1] | ¹H NMR (DMSO-d6, 400 MHz) δ 9.50 (s, 1H), 9.46-9.08 (m, 1H), 8.55 (s, 1H), 7.96 (d, J = 9.6 Hz 1H), 7.92-7.87 (m, 1H), 7.80 (t, J = 7.8 Hz, 1H), 7.55 ( d, J = 8.0 Hz, 1H), 7.27 (d, J = 7.6 Hz, 1H), 4.95-4.36 (m, 1H), 3.87 (s, 1H), 3.43-3.39 (m, 1H), 3.22-3.10 (m, 1H), 2.53 (s, 3H), 2.33-2.30 (m, 1H), 1.94-1.71 (m, 5H) | 360.0 |
| 1.50 | 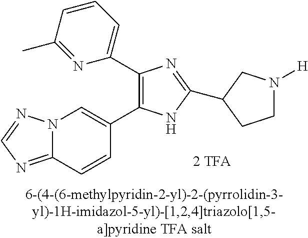 [SMILES: Cc1cccc(-c2nc(C3CCNC3)[nH]c2-c2ccc3ncnn3c2)n1] | ¹H NMR (DMSO-d₆, 400 MHz) δ 9.47 (s, 1H), 9.27 (s, 2H), 8.59 (s, 1H), 7.94-7.85 (m, 3H), 7.53 (d, J = 8.0 Hz, 2H), 7.38 (d, J = 7.6 Hz, 2H), 3.85-3.69 (m, 1H), 3.58-3.28 (m, 4H), 2.57 (s, 3H), 2.48-2.42 (m, 1H), 2.36-2.26 (m, 1H) | 346.1 |
| 1.51 | 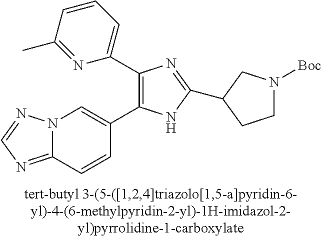 [SMILES: Cc1cccc(-c2nc(C3CCN(C(=O)OC(C)(C)C)C3)[nH]c2-c2ccc3ncnn3c2)n1] | | 468.2 (M + Na) |
| 1.52 | 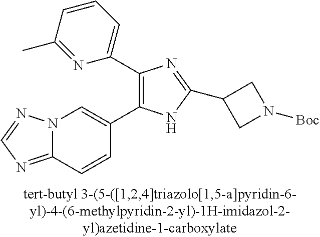 [SMILES: Cc1cccc(-c2nc(C3CN(C(=O)OC(C)(C)C)C3)[nH]c2-c2ccc3ncnn3c2)n1] | | 454.2 |
| 1.53 | 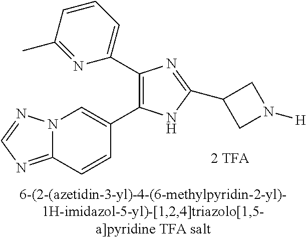 [SMILES: Cc1cccc(-c2nc(C3CNC3)[nH]c2-c2ccc3ncnn3c2)n1] | | 332.1 |
| 1.54 | 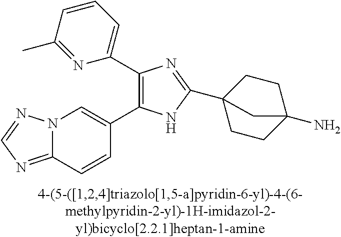 [SMILES: Cc1cccc(-c2nc(C34CCC(N)(CC3)C4)[nH]c2-c2ccc3ncnn3c2)n1] | | 386.2 |
| 1.55 | 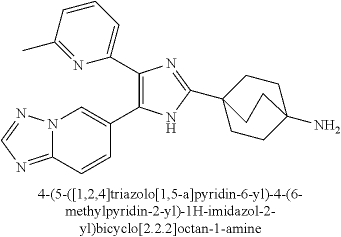 [SMILES: Cc1cccc(-c2nc(C34CCC(N)(CC3)CC4)[nH]c2-c2ccc3ncnn3c2)n1] | ¹H NMR (MeOD, 400 MHz) δ 9.22 (s, 1H), 8.55 (s, 1H), 7.97-7.88 (m, 2H), 7.80 (dd, J = 1.6, J = 9.2 Hz, 1H), 7.51-7.48 (m, 2H), 2.69 (s, 3H), 2.37-2.26 (m, 6H), 2.09-1.97 (m, 6H) | 400.3 |
| 1.56 | 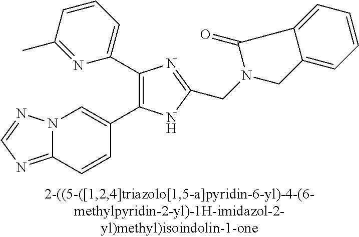 [SMILES: Cc1cccc(-c2nc(CN3Cc4ccccc4C3=O)[nH]c2-c2ccc3ncnn3c2)n1] | ¹H NMR (MeOD, 400 MHz) δ 9.18 (d, J = 1.6 Hz, 1H), 8.50 (s, 1H), 7.90 (t, J = 8.0 Hz 1H), 8.82 (s, 2H), 7.89-7.80 (m, 3H), 7.67-7.60 (m, 2H), 7.56-7.47 (m, 3H), 5.09 (s, 2H), 4.71 (s, 2H), 2.69 (s, 3H) | 422.2 |
| 1.57 | 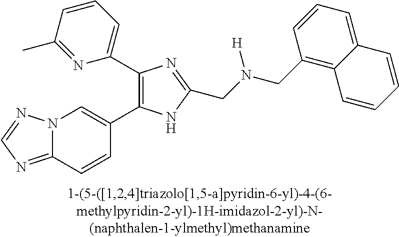 [SMILES: Cc1cccc(-c2nc(CNCc3cccc4ccccc34)[nH]c2-c2ccc3ncnn3c2)n1] | ¹H NMR (MeOD, 400 MHz): δ 9.14 (d, J = 1.2 Hz, 1H), 8.42 (s, 1H), 8.15 (d, J = 8.0 Hz, 1H), 7.87-7.81 (m, 2H), 7.78-7.73 (m, 2H), 7.69- 7.62 (m, 1H), 7.55-7.37 (m, 5H), 7.34-7.29 (m, 1H), 7.19-7.15 (m, 1H), 4.32 (s, 2H), 4.05 (s, 2H), 2.51 (s, 3H) | 446.2 |
| 1.58 | 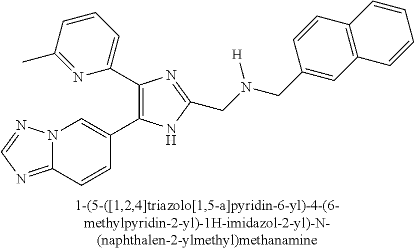 [SMILES: Cc1cccc(-c2nc(CNCc3ccc4ccccc4c3)[nH]c2-c2ccc3ncnn3c2)n1] | ¹H NMR (MeOD, 400 MHz) δ 9.16 (s, 1H), 8.51 (s, 3H), 8.06 (s, 2H), 7.97-7.85 (m, 4H), 7.85-7.84 (m, 2H), 7.64-7.58(m, 1H), 7.57-.55 (m, 3H), 7.55-7.53 (m, 1H), 4.65 (s, 2H), 4.51 (s, 2H), 2.70 (s, 3H) | 446.2 |
| 1.59 | 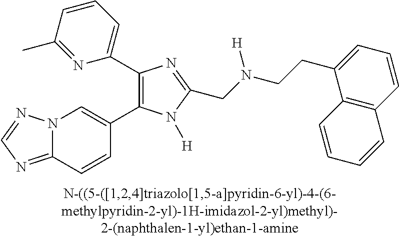 [SMILES: Cc1cccc(-c2nc(CNCCc3cccc4ccccc34)[nH]c2-c2ccc3ncnn3c2)n1] | ¹H NMR (MeOD, 400 MHz) δ 9.13 (s, 1H), 8.43 (s, 1H), 8.08 (d, J = 7.4 Hz, 1H), 7.83-7.81 (m, 2H), 7.76- 7.70 (m, 2H), 7.65 (t, J = 7.8 Hz, 1H), 7.46-7.41 (m, 2H), 7.38 (d, J = 5.4 Hz, 2H), 7.32 (d, J = 7.8 Hz, 1H), 7.18 (d, J = 7.8 Hz, 1H), 3.98 (s, 1H), 3.35-3.33 (m, 2H), 3.04 (t, J = 7.4 Hz, 2H), 2.51 (s, 3H) | 460.3 |
| 1.60 | 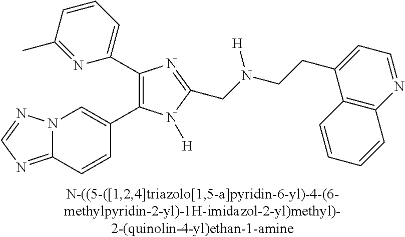 [SMILES: Cc1cccc(-c2nc(CNCCc3ccnc4ccccc34)[nH]c2-c2ccc3ncnn3c2)n1] | ¹H NMR (MeOD, 400 MHz) δ 9.15 (s, 1H), 8.73 (d, J = 4.4 Hz, 1H), 8.44 (s, 1H), 8.21 (d, J = 8.4 Hz, 1H), 8.01 (d, J = 8.4 Hz, 1H), 7.84 (dd, J = 1.6, 9.2 Hz, 1H), 7.78-7.58 (m, 4H), 7.46 (d, J = 4.4 Hz, 1H), 7.33 (d, J = 8.0 Hz, 1H), 7.18 (d, J = 7.6 Hz, 1H), 4.00 (s. 2H), 3.40 (t, J = 7.6 Hz, 2H), 3.12 (t, J = 7.6 Hz, 2H), 2.52 (s, 3H) | 461.3 |
| 1.61 | 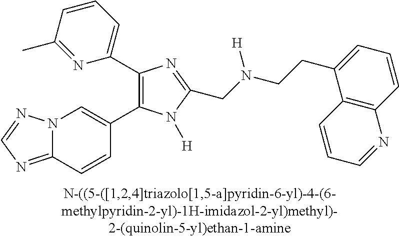 [SMILES: Cc1cccc(-c2nc(CNCCc3cccc4ncccc34)[nH]c2-c2ccc3ncnn3c2)n1] | ¹H NMR (DMSO-d6, 400 MHz) δ 9.21 (s, 1H), 8.84 (d, J = 2.8 Hz, 1H), 8.65 (d, J = 8.4 Hz, 1H), 8.45 (s, 1H), 7.96 (d, J = 8.4 Hz, 1H), 7.88 (dd, J = 1.6, 9.2 Hz, 1H), 7.79- 7.66 (m, 3H), 7.59 (d, J = 7.2 Hz, 1H), 7.55 (dd, J = 4.4, 8.4 Hz, 1H), 7.38 (d, J = 7.6 Hz, 1H),7.21 (d, J = 7.6 Hz, 1H), 4.27 (s. 2H), 3.51 (t, J = 7.6 Hz, 2H), 3.36 (t, J = 7.6 Hz, 2H), 2.54 (s, 3H) | 461.3 |
| 1.63 | 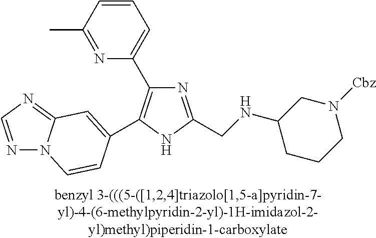 [SMILES: Cc1cccc(-c2nc(CNC3CCCN(C(=O)OCc4ccccc4)C3)[nH]c2-c2ccn3ncnc3c2)n1] | | 523.2 |
| 1.64 | 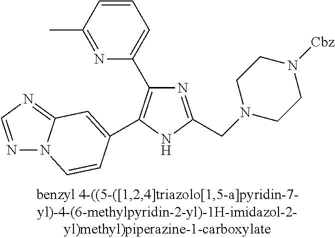 [SMILES: Cc1cccc(-c2nc(CN3CCN(C(=O)OCc4ccccc4)CC3)[nH]c2-c2ccn3ncnc3c2)n1] | | 509.2 |
| 1.65 | 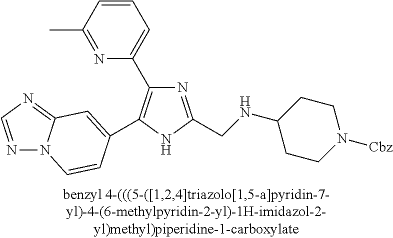 [SMILES: Cc1cccc(-c2nc(CNC3CCN(C(=O)OCc4ccccc4)CC3)[nH]c2-c2ccn3ncnc3c2)n1] | | 523.2 |
| 1.66 | 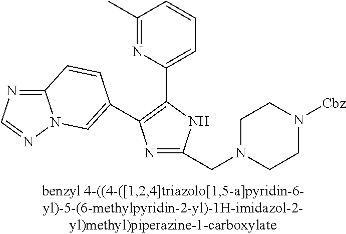 [SMILES: Cc1cccc(-c2[nH]c(CN3CCN(C(=O)OCc4ccccc4)CC3)nc2-c2ccc3ncnn3c2)n1] | | 509.2 |

### Example 3

### Linking Antibody Constructs to TGFbR1 Inhibitors Via a Linker

This example shows different methods of linking an antibody construct to a TGFβR1 inhibitor via a linker to form a conjugate.

A linker, such as a maleimidocaproyl)-(valine-citrulline)-(para-aminobenzyloxycarbonyl) linker or disulfide linker (e.g., formula Id-If) can be first attached to a TGFβR1 inhibitor to form a TGFβR1 inhibitor-linker compound. Subsequently, a TGFβR1 inhibitor-linker is conjugated to an antibody construct.

Alternatively, a linker is attached to an antibody construct, in which the linker is a disulfide linker (e.g., in formula Id-If) or a hydrazone linker to form a linker-antibody construct. Subsequently, a TGFβR1 inhibitor is conjugated to the linker linked with the antibody construct.

### Example 4

### Lysine-Based Bioconjugation

The antibody construct is exchanged into an appropriate buffer, for example, phosphate, borate, PBS, or Tris-Acetate, at a concentration of about 2 mg/mL to about 10 mg/mL. An appropriate number of equivalents of the TGFβR1 inhibitor-linker are added as a solution with stirring. Under some conditions, lysine-linked conjugates are synthesized starting with 10 mg of antibody construct (mAb) and 10 equivalents of TGFβR1 inhibitor-linker, using sodium phosphate buffer at pH 8 in 20% v/v DMSO. Dependent on the physical properties of the TGFβR1 inhibitor-linker construct, a co-solvent can be introduced prior to the addition of the TGFβR1 inhibitor-linker construct to facilitate solubility. The reaction is stirred at room temperature for 2 hours to about 12 hours depending on the observed reactivity. The progression of the reaction is monitored by LC-MS. Once the reaction is deemed complete, the remaining TGFβR1 inhibitor-linker constructs are removed by applicable methods and the lysine-linked TGFβR1 inhibitor conjugate is exchanged into the desired formulation buffer. Monomer content and drug-antibody ratios are determined by methods described herein.

### Example 5

### Cysteine-Based Bioconjugation to Interchain Disulfides

The antibody (e.g., 10 mg) is exchanged into an appropriate buffer, for example, phosphate, borate, PBS, or Tris-Acetate, at pH 8, and at a concentration of about 2 mg/mL to about 10 mg/mL in 20% v/v DMSO, with an appropriate number of equivalents of a reducing agent, for example, dithiothreitol or tris(2-carboxyethyl)phosphine. The resultant solution is stirred for an appropriate amount of time and temperature to effect the desired reduction. The TGFβR1 inhibitor-linker construct (e.g., 7 equiv.) is added as a solution with stirring. Dependent on the physical properties of the TGFβR1 inhibitor-linker construct, a co-solvent is introduced prior to the addition of the TGFβR1 inhibitor-linker construct to facilitate solubility. The reaction is stirred at room temperature for about 1 hour to about 12 hours depending on the observed reactivity. The progression of the reaction is monitored by liquid chromatography-mass spectrometry (LC-MS). Once the reaction is deemed complete, the remaining free immune stimulatory compound-linker construct is removed by applicable methods and the conjugate is exchanged into the desired formulation buffer. Monomer content and drug-antibody ratios can be determined by methods described herein.

### Example 6

### Conjugation to Engineered/Site-Specific Cysteines

The antibody construct (e.g., 10 mg) is exchanged into an appropriate buffer, for example, phosphate, HEPES, borate, PBS, or Tris-Acetate, at a concentration of about 2 mg/mL to about 10 mg/mL. To the solution is added an appropriate number of equivalents (10 to about 40) of an appropriate reducing agent, for example, dithiothreitol or tris(2-carboxyethyl)phosphine. The reaction is then incubated at room temperature for about 1 hour to about 12 hours depending on the observed reactivity. The reduced antibody construct is then exchanged into an appropriate buffer, for example, phosphate, HEPES, borate, PBS, or Tris-Acetate, at a concentration of about 2 mg/mL to about 10 mg/mL (for the purposes of removing the previously described reducing agent). To the solution is added an appropriate number of equivalents (10 to about 40) of an appropriate oxidizing agent, for example, dehydroascorbic acid. The reaction is then incubated at room temperature for about 1 hour to about 5 hours depending on the observed reactivity. The TGFβR1 inhibitor-linker construct (e.g., 7 equiv.) is added as a solution with stirring. Dependent on the physical properties of the compound-linker construct, a co-solvent can be introduced prior to the addition of the compound-linker construct to facilitate solubility. The reaction is stirred at room temperature for 2 hours to about 12 hours depending on the observed reactivity. The progression of the reaction is monitored by LC-MS, hydrophobic interaction chromatography, or other appropriate means. Once the reaction is deemed complete, the remaining TGFβR1 inhibitor-linker constructs are removed by applicable methods and the site-specific cysteine-linked conjugate is exchanged into the desired formulation buffer. Monomer content and drug-antibody ratios can be determined by methods described herein and known in the art.

### Example 7

### Production of Exemplary ASGR1 Antibodies

ASGR1 4A2 Heavy and Light chain DNA was cloned into separate expression vectors for human IgG1 antibody production. The heavy chain vector construct and the light chain vector construct were transiently co-expressed in the ExpiCHO™ system to generate 4A2 IgG1 antibody. The protein from the harvested ExpiCHO™ supernatant was purified to homogeneity using protein A column on GE AKTA Pure™ system and confirmed for purity using analytical HPLC-SEC. If needed, additional purification was performed on a preparative SEC column to remove aggregates. ASGR1 72G9 and 176H4 IgG1 antibodies were created in the same manner. The sequences for the heavy chain and lights chains for the 4A2, 72G9 and 176H4 antibodies are set forth in WO2017/058944, which is incorporated herein by reference in its entirety and for all purposes.

Additionally, hybridomas producing monoclonal antibodies (mAbs) specific for human ASGR1 were prepared from Balb/C mice immunized with ASGR1-AviHis at a service provider using their propriety protocol. Antibody heavy and light chain sequences were obtained, amplified, and cloned. Clone supernatants containing the expressed mAbs were screened for binding to a human ASGR1-IgG1Fc fusion protein, and those with a positive signal as measured by enzyme linked immunosorbent assay (ELISA) were screened for cell binding on HepG2 cells (which express ASGR1). Based on the initial selection criteria, sixteen (16) mAbs were expressed and isolated. The isolated mAbs were further analyzed for the additional characteristics including, for example, titer, low aggregate content following protein A purification, high titer, lack of cross-blocking of ligand GalNac, calcium sensitivity, and cross-reactivity with rat ASGR1 protein. A total of five (5) clones (G2D, K2E, J4F, L4L and H8K) exhibiting strong signals in these assays were humanized and subjected to epitope binning (see Example 2) as compared to known anti-ASGR mAbs 4A2, 72G9 and 4F3 (see, e.g., PCT Publication Nos. WO 2017/058944 and WO 2014/023709), referred to herein as ASGR1 mAb-A, ASGR1 mAb-B, and ASGR1 mAb-C, respectively.

### mAb G2D Humanization

For humanization of the G2D VH region (SEQ ID NO:38), the 3 CDR loops as defined by Kabat were grafted into the human germline sequence VH1-03 with JH6 to generate hzG2D VH (SEQ ID NO:43). In addition, several variants of hzG2D VH were constructed to contain one or more mouse back mutations in framework region 1 (FR1), in CDR2, at the junction of framework region 3 (FR3) with CDR2, in FR3, or any combination thereof (see SEQ ID NOS:44-51 and 82-88). Several of the humanized heavy chains were further changed by adding mutations in CDR2, namely N54Q or G55A, to remove a potential deamidation site (see SEQ ID NOS:52-81).

For humanization of the G2D VL region (SEQ ID NO:126) of clones 1-16, 19-21, 26-29, 31, and 33, the 3 CDR loops as defined by Kabat were grafted into the human germline sequence VKI-L1 with JK4 to generate hzG2D VLₐ (SEQ ID NO:131). In addition, mutations were introduced into humanized light chains at the CDR2/FR3 junction, namely D56S or D56E, to remove a potential isomerization site (see SEQ ID NOS:132-133).

For humanization of the G2D VL region (SEQ ID NO:126) of clones 22, 23, 30, 32, and 34-37, the 3 CDR loops as defined by Kabat were grafted into the human germline sequence VKI-O2 with JK4 to generate hzG2D VL_{b} (SEQ ID NO:247). In addition, a mutation was introduced into humanized light chains at the CDR2/FR3 junction, namely D56S, to remove a potential isomerization site (see SEQ ID NO:248).

For humanization of the G2D VL region (SEQ ID NO:126) of clones 24 and 25, the 3 CDR loops as defined by Kabat were grafted into the human germline sequence VKI-A20 with JK4 to generate hzG2D VL_{c} (SEQ ID NO:249).

Variable heavy region sequences were cloned into a vector containing a signal peptide sequence and human IgG1 constant region (SEQ ID NO:230), while variable light chain regions were cloned into a vector containing a signal peptide sequence and a human kappa light chain constant region (SEQ ID NO:232). A total of twenty two humanized heavy chains were paired with six humanized light chains to generate mAbs analyzed for the desirable characteristics noted above.

TABLE 5
Characterization of humanized G2D clones

| | | | | | Guava | |
|---|---|---|---|---|---|---|
| | | | Titer | % | HepG2 | HepG2 |
| Name | VH (IgG1) | VL (kappa) | mg/L | POI | Screen | EC₅₀ |
| G2D-C | G2D VH | G2D VL | 250 | 98 | 187/145 | 0.4 |
| | (SEQ ID NO: 38) | (SEQ ID NO: 126) | | | | |
| hzG2D-1 | hzG2D VH V109L | hzG2D VLₐ | 9 | | 32/19 | |
| | (SEQ ID NO: 43) | (SEQ ID NO: 131) | | | | |
| hzG2D-2 | hzG2.1D VH T28S, | hzG2D VLₐ | 19 | | 38/20 | |
| | V109L | (SEQ ID NO: 131) | | | | |
| | (SEQ ID NO: 44) | | | | | |
| hzG2D-3 | hzG2.45D VH A93T, | hzG2D VLₐ | 59 | | 131/95 | |
| | R94S, V109L | (SEQ ID NO: 131) | | | | |
| | (SEQ ID NO: 88) | | | | | |
| hzG2D-4 | hzG2.39D VH R66K, | hzG2D VLₐ | 12 | | 55/22 | |
| | V67A, V109L | (SEQ ID NO: 131) | | | | |
| | (SEQ ID NO: 82) | | | | | |
| hzG2D-5 | hzG2.8D VH T28S, | hzG2D VLₐ | 75 | | 91/55 | |
| | A93T, R94S, V109L | (SEQ ID NO: 131) | | | | |
| | (SEQ ID NO: 51) | | | | | |
| hzG2D-6 | hzG2.43D VH T68S, | hzG2D VLa | 24 | | 39/23 | |
| | I69L, R71V, V109L | (SEQ ID NO: 131) | | | | |
| | (SEQ ID NO: 86) | | | | | |
| hzG2D-7 | hzG2.2D VH T28S, | hzG2D VLa | 12 | | 46/22 | |
| | R66K, V67A, V109L | (SEQ ID NO: 131) | | | | |
| | (SEQ ID NO: 45) | | | | | |
| hzG2D-8 | hzG2.6D VH T28S, | hzG2D VLa | 23 | | 31/21 | |
| | T68S, I69L, R71V, | (SEQ ID NO: 131) | | | | |
| | V109L | | | | | |
| | (SEQ ID NO: 49) | | | | | |
| hzG2D-9 | hzG2.40D VH R66K, | hzG2D VLₐ | 23 | 81 | 155/117 | |
| | V67A, A93T, R94S, | (SEQ ID NO: 131) | | | | |
| | V109L | | | | | |
| | (SEQ ID NO: 83) | | | | | |
| hzG2D-10 | hzG2.44D VH T68S, | hzG2D VLₐ | 102 | 30 | 149/63 | |
| | I69L, R71V, A93T, | (SEQ ID NO: 131) | | | | |
| | R94S, V109L | | | | | |
| | (SEQ ID NO: 87) | | | | | |
| hzG2D-11 | hzG2.41D VH R66K, | hzG2D VLₐ | 10 | 68 | Very | |
| | V67A, T68S, I69L, | (SEQ ID NO: 131) | | | low | |
| | R71V, V109L | | | | | |
| | (SEQ ID NO: 84) | | | | | |
| hzG2D-12 | hzG2.3D VH T28S, | hzG2D VLₐ | 19 | 67 | 158/113 | |
| | R66K, V67A, A93T, | (SEQ ID NO: 131) | | | | |
| | R94S, V109L | | | | | |
| | (SEQ ID NO: 46) | | | | | |
| hzG2D-13 | hzG2.7D VH T28S, | hzG2D VLₐ | 54 | 46 | 140/83 | |
| | T68S, I69L, R71V, | (SEQ ID NO: 131) | | | | |
| | A93T, R94S, V109L | | | | | |
| | (SEQ ID NO: 50) | | | | | |
| hzG2D-14 | hzG2.4D VH T28S, | hzG2D VLₐ | 10 | | 37/16 | |
| | R66K, V67A, T68S, | (SEQ ID NO: 131) | | | | |
| | I69L, R71V, V109L | | | | | |
| | (SEQ ID NO: 47) | | | | | |
| hzG2D-15 | hzG2.42D VH R66K, | hzG2D VLₐ | 45 | 45 | 172/119 | |
| | V67A, T68S, I69L, | (SEQ ID NO: 131) | | | | |
| | R71V, A93T, R94S, | | | | | |
| | V109L | | | | | |
| | (SEQ ID NO: 85) | | | | | |
| hzG2D-16 | hzG2.5D VH T28S, | hzG2D VLₐ | 39 | 40 | 165/108 | |
| | R66K, V67A, T68S, | (SEQ ID NO: 131) | | | | |
| | I69L, R71V, A93T, | | | | | |
| | R94S, V109L | | | | | |
| | (SEQ ID NO: 48) | | | | | |
| hzG2D-17 | hzG2.41D VH R66K, | G2D VL | 80 | | Very | |
| | V67A, T68S, I69L, | (SEQ ID NO: 126) | | | low | |
| | R71V, V109L | | | | | |
| | (SEQ ID NO: 84) | | | | | |
| hzG2D-18 | hzG2.42D VH R66K, | G2D VL | 206 | 76 | ND | |
| | V67A, T68S, I69L, | (SEQ ID NO: 126) | | | | |
| | R71V, A93T, R94S, | | | | | |
| | V109L | | | | | |
| | (SEQ ID NO: 85) | | | | | |
| hzG2D-19 | G2D VH | hzG2D VLa | 33 | 90 | ND | |
| | (SEQ ID NO: 38) | (SEQ ID NO: 131) | | | | |
| hzG2D-20 | hzG2.46D VH R66K, | hzG2D VLₐ | 30 | | Very | |
| | V67A, T68S, I69L, | (SEQ ID NO: 131) | | | low | |
| | R71V | | | | | |
| | (SEQ ID NO: 238) | | | | | |
| hzG2D-21 | hzG2.47D VH R66K, | hzG2D VLₐ | 40 | | 199/127 | |
| | V67A, T68S, I69L, | (SEQ ID NO: 131) | | | | |
| | R71V, A93T, R94S | | | | | |
| | (SEQ ID NO: 239) | | | | | |
| hzG2D-22 | hzG2.46D VH R66K, | hzG2D VL_{b} | 115 | | None | |
| | V67A, T68S, I69L, | (SEQ ID NO: 247) | | | | |
| | R71V | | | | | |
| | (SEQ ID NO: 238) | | | | | |
| hzG2D-23 | hzG2.47D VH R66K, | hzG2D VL_{b} | 215 | 98 | 198/131 | 0.3 |
| | V67A, T68S, I69L, | (SEQ ID NO: 247) | | | | |
| | R71V, A93T, R94S | | | | | |
| | (SEQ ID NO: 239) | | | | | |
| hzG2D-24 | hzG2.46D VH R66K, | hzG2D VL_{c} | 3 | | None | |
| | V67A, T68S, I69L, | (SEQ ID NO: 249) | | | | |
| | R71V | | | | | |
| | (SEQ ID NO: 238) | | | | | |
| hzG2D-25 | hzG2.47D VH R66K, | hzG2D VL_{c} | 58 | | None | |
| | V67A, T68S, I69L, | (SEQ ID NO: 249) | | | | |
| | R71V, A93T, R94S | | | | | |
| | (SEQ ID NO: 239) | | | | | |
| hzG2D-26 | hzG2.47D VH R66K, | hzG2.3D VLₐ K45E, | 72 | 93 | 185/114 | |
| | V67A, T68S, I69L, | S46T | | | | |
| | R71V, A93T, R94S | (SEQ ID NO: 244) | | | | |
| | (SEQ ID NO: 239) | | | | | |
| hzG2D-27 | hzG2.47D VH R66K, | hzG2.4D VLₐ 79E, | 165 | 98 | 191/116 | |
| | V67A, T68S, I69L, | P80Y | | | | |
| | R71V, A93T, R94S | (SEQ ID NO: 245) | | | | |
| | (SEQ ID NO: 239) | | | | | |
| hzG2D-28 | hzG2.47D VH R66K, | hzG2.5D VLₐ F71Y, | 72 | 66 | 145/92 | |
| | V67A, T68S, I69L, | T72S | | | | |
| | R71V, A93T, R94S | (SEQ ID NO: 246) | | | | |
| | (SEQ ID NO: 239) | | | | | |
| hzG2D-29 | hzG2.49D VH R38K, | hzG2D VLₐ | 57 | 72 | 189/108 | |
| | R66K, V67A, T68S, | (SEQ ID NO: 131) | | | | |
| | I69L, R71V, A93T, | | | | | |
| | R94S | | | | | |
| | (SEQ ID NO: 241) | | | | | |
| hzG2D-30 | hzG2.49D VH R38K, | hzG2D VL_{b} | 189 | 96 | 198/115 | |
| | R66K, V67A, T68S, | (SEQ ID NO: 247) | | | | |
| | I69L, R71V, A93T, | | | | | |
| | R94S | | | | | |
| | (SEQ ID NO: 241) | | | | | |
| hzG2D-31 | hzG2.50D VH R66K, | hzG2D VLₐ | 41 | 73 | 168/95 | |
| | V67A, T68S, I69L, | (SEQ ID NO: 131) | | | | |
| | R71V, L82F, S82aH, | | | | | |
| | A93T, R94S | | | | | |
| | (SEQ ID NO: 242) | | | | | |
| hzG2D-32 | hzG2.50D VH R66K, | hzG2D VL_{b} | 153 | 94 | 167/103 | |
| | V67A, T68S, I69L, | (SEQ ID NO: 247) | | | | |
| | R71V, L82F, S82aH, | | | | | |
| | A93T, R94S | | | | | |
| | (SEQ ID NO: 242) | | | | | |
| hzG2D-33 | hzG2.51D VH R66K, | hzG2D VLₐ | 60 | 61 | 164/85 | |
| | V67A, T68S, I69L, | (SEQ ID NO: 131) | | | | |
| | R71V, R84T, A93T, | | | | | |
| | R94S | | | | | |
| | (SEQ ID NO: 243) | | | | | |
| hzG2D-34 | hzG2.51D VH R66K, | hzG2D VL_{b} | 198 | 86 | 194/109 | |
| | V67A, T68S, I69L, | (SEQ ID NO: 247) | | | | |
| | R71V, R84T, A93T, | | | | | |
| | R94S | | | | | |
| | (SEQ ID NO: 243) | | | | | |
| hzG2D-35 | hzG2.47D VH R66K, | hzG2.6D VL_{b} D56S | 262 | 99 | ND | 0.3 |
| | V67A, T68S, I69L, | (SEQ ID NO: 248) | | | | |
| | R71V, A93T, R94S | | | | | |
| | (SEQ ID NO: 239) | | | | | |
| hzG2D-36 | hzG2.48D VH G55A, | hzG2D VL_{b} | 94 | 82 | ND | 0.3 |
| | R66K, V67A, T68S, | (SEQ ID NO: 247) | | | | |
| | I69L, R71V, A93T, | | | | | |
| | R94S | | | | | |
| | (SEQ ID NO: 240) | | | | | |
| hzG2D-37 | hzG2.48D VH G55A, | hzG2.6D VL_{b} D56S | 403 | 98 | ND | 0.3 |
| | R66K, V67A, T68S, | (SEQ ID NO: 248) | | | | |
| | I69L, R71V, A93T, | | | | | |
| | R94S | | | | | |
| | (SEQ ID NQ: 240) | | | | | |

### mAb K2E Humanization

Germline VH1-46 with JH6 was used for CDR grafting the variable heavy chain (SEQ ID NO:39) and germline VKI-L1 with JK2 was used for CDR grafting the variable light chain (SEQ ID NO:127). CDR grafting was done using Kabat defined CDRs to generate hzK2E VH (SEQ ID NO:89) and hzK2E VL (SEQ ID NO:134). Several variants of hzK2E VH (SEQ ID NOS:90-93) and hzK2E VL (SEQ ID NO:135) containing mouse framework back mutations were generated, and the sequences determined using a 3D structural model for potential influence of residues on the CDR structure. In addition, mutations were introduced into humanized light chains at the CDR2/FR3 junction, namely D56E, to remove a potential isomerization site (see SEQ ID NOS:136-137). Variable heavy region sequences were cloned into a vector containing a signal peptide sequence and IgG1 constant region. Variable light regions were cloned into a vector containing a signal peptide sequence and kappa constant region.

Five humanized heavy chains were individually paired with each of four different humanized light chains to generate mAbs analyzed for the desirable characteristics noted above (see Table 6). The humanized sequences were further modified by adding an additional mutation in the light chain CDR2/FR3 junction, namely D56E, to remove a potential isomerization site.

TABLE 6
Characterization of humanized K2E clones

| | | | Titer | % | HepG2 |
|---|---|---|---|---|---|
| Name | VH (IgG1) | VL (kappa) | mg/L | POI | EC₅₀ |
| K2E-C | K2E VH | K2E VL | 512 | 99 | 0.50 |
| | (SEQ ID NO: 39) | (SEQ ID NO: 127) | | | |
| hzK2E-1 | hzK2E VH | hzK2E VL | 479 | 100 | 0.53 |
| | (SEQ ID NO: 89) | (SEQ ID NO: 134) | | | |
| hzK2E-2 | hzK2.1E VH R66K, | hzK2E VL | 445 | 99 | 0.41 |
| | V67T | (SEQ ID NO: 134) | | | |
| | (SEQ ID NO: 90) | | | | |
| hzK2E-3 | hzK2.4E VH M69L, | hzK2E VL | 455 | 99 | 0.68 |
| | R71A | (SEQ ID NO: 134) | | | |
| | (SEQ ID NO: 93) | | | | |
| hzK2E-4 | hzK2.2E VH R66K, | hzK2E VL | 401 | 100 | 0.90 |
| | V67T, M69L, R71A | (SEQ ID NO: 134) | | | |
| | (SEQ ID NO: 91) | | | | |
| hzK2E-5 | hzK2.3E VH R66K, | hzK2E VL | 386 | 100 | 0.54 |
| | V67T, M69L, R71A, | (SEQ ID NO: 134) | | | |
| | T73K | | | | |
| | (SEQ ID NO: 92) | | | | |
| hzK2E-6 | hzK2E VH | hzK2.1E VL S46T | 435 | 99 | |
| | (SEQ ID NO: 89) | (SEQ ID NO: 135) | | | |
| hzK2E-7 | hzK2.1E VH R66K, | hzK2.1E VL S46T | 440 | 100 | |
| | V67T | (SEQ ID NO: 135) | | | |
| | (SEQ ID NO: 90) | | | | |
| hzK2E-8 | hzK2.4E VH M69L, | hzK2.1E VL S46T | 440 | 99 | |
| | R71A | (SEQ ID NO: 135) | | | |
| | (SEQ ID NO: 93) | | | | |
| hzK2E-9 | hzK2.2E VH R66K, | hzK2.1E VL S46T | 422 | 99 | |
| | V67T, M69L, R71A | (SEQ ID NO: 135) | | | |
| | (SEQ ID NO: 91) | | | | |
| hzK2E-10 | hzK2.3E VH R66K, | hzK2.1E VL S46T | 451 | 100 | 0.50 |
| | V67T, M69L, R71A, | (SEQ ID NO: 135) | | | |
| | T73K | | | | |
| | (SEQ ID NO: 92) | | | | |
| hzK2E-11 | hzK2E VH | hzK2.2E VL D56E | 479 | 99 | 0.46 |
| | (SEQ ID NO: 89) | (SEQ ID NO: 136) | | | |
| hzK2E-12 | hzK2.1E VH R66K, | hzK2.2E VL D56E | 393 | 393 | 0.45 |
| | V67T | (SEQ ID NO: 136) | | | |
| | (SEQ ID NO: 90) | | | | |
| hzK2E-13 | hzK2.4E VH M69L, | hzK2.2E VL D56E | 389 | 389 | 0.92 |
| | R71A | (SEQ ID NO: 136) | | | |
| | (SEQ ID NO: 93) | | | | |
| hzK2E-14 | hzK2.2E VH R66K, | hzK2.2E VL D56E | 411 | 411 | 0.77 |
| | V67T, M69L, R71A | (SEQ ID NO: 136) | | | |
| | (SEQ ID NO: 91) | | | | |
| hzK2E-15 | hzK2.3E VH R66K, | hzK2.2E VL D56E | 427 | 427 | |
| | V67T, M69L, R71A, | (SEQ ID NO: 136) | | | |
| | T73K | | | | |
| | (SEQ ID NO: 92) | | | | |
| hzK2E-16 | hzK2E VH | hzK2.3E VL S46T | 432 | 432 | |
| | (SEQ ID NO: 89) | D56E | | | |
| | | (SEQ ID NO: 137) | | | |
| hzK2E-17 | hzK2.1E VH R66K, | hzK2.3E VL S46T | 384 | 384 | |
| | V67T | D56E | | | |
| | (SEQ ID NO: 90) | (SEQ ID NO: 137) | | | |
| hzK2E-18 | hzK2.4E VH M69L, | hzK2.3E VL S46T | 380 | 380 | |
| | R71A | D56E | | | |
| | (SEQ ID NO: 93) | (SEQ ID NO: 137) | | | |
| hzK2E-19 | hzK2.2E VH R66K, | hzK2.3E VL S46T | 361 | 361 | |
| | V67T, M69L, R71A | D56E | | | |
| | (SEQ ID NO: 91) | (SEQ ID NO: 137) | | | |
| hzK2E-20 | hzK2.3E VH R66K, | hzK2.3E VL S46T | 363 | 363 | |
| | V67T, M69L, R71A, | D56E | | | |
| | T73K | (SEQ ID NO: 137) | | | |
| | (SEQ ID NO: 92) | | | | |

### mAb L4L Humanization

Germline VH4-31 with JH6 was used for CDR grafting the variable heavy chain (SEQ ID NO:40) and germline VKI-O2 with JK4 were used for CDR grafting the variable light chain (SEQ ID NO: 128). CDR grafting was done using Kabat defined CDRs to generate hzL4L VH (SEQ ID NO: 94) and hzL4L VL (SEQ ID NO:138). Several variants of hzL4L VH (SEQ ID NOS:95-102) containing mouse framework back mutations were generated, and the sequences determined using a 3D structural model for potential influence of residues on the CDR structure. Variable heavy region sequences were cloned into a vector containing a signal peptide sequence and IgG1 constant region. Variable light regions were cloned into a vector containing a signal peptide sequence and kappa constant region.

Nine humanized heavy chains were individually co-transfected individually with four different humanized light chains (see Table 7) into the ExpiCHO™ expression system in a 30 mL culture, using the parental chimeric antibody, L4L, as a benchmark. The supernatant containing the antibody was purified and further characterized. Humanized sequences hzL4L-10 through hzL4L-14 were further modified by adding additional mutations in the light chain CDR2/FR3 junction, namely D56S (SEQ ID NO:139), D56E (SEQ ID NO:140) or G57A (SEQ ID NO:141), to remove a potential isomerization site. There was a reduction in percent purity, as measured by analytical size-exclusion chromatography (% protein of interest (POI)), seen with the chimeric and hzL4L-1, -2 and -6. Surprisingly, the G27Y mutation appears to help stabilize the structure, resulting in a more homogenous recovery (see Table 7).

TABLE 7
Characterization of humanized L4L clones

| | | | | | Guava | Flow |
|---|---|---|---|---|---|---|
| | | | Titer | % | HepG2 | HepG2 |
| Name | VH (IgG1) | VL (kappa) | mg/L | POI | Screen | EC₅₀ |
| L4L | L4L VH | L4L VL | 327 | 65 | Good | 1.9 |
| | (SEQ ID NO: 40) | (SEQ ID NO: 128) | | | | |
| hzL4L-1 | hzL4L VH | hzL4L VL | 462 | 72 | Poor | |
| | (SEQ ID NO: 94) | (SEQ ID NO: 138) | | | | |
| hzL4L-2 | hzL4L VH S30T, | hzL4L VL | 495 | 71 | Good | |
| | V67I, T68S, 70T, | (SEQ ID NO: 138) | | | | |
| | V71R | | | | | |
| | hzL4.1L | | | | | |
| | (SEQ ID NO: 95) | | | | | |
| hzL4L-3 | hzL4L VH G27Y, | hzL4L VL | 289 | 98 | Good | 1.5 |
| | S30T, V67I, T68S, | (SEQ ID NO: 138) | | | | |
| | S70T, V71R | | | | | |
| | hzL4.2L VH | | | | | |
| | (SEQ ID NO: 96) | | | | | |
| hzL4L-4 | hzL4L VH G27Y, | hzL4L VL | 337 | 91 | Poor | 1.8 |
| | S30T | (SEQ ID NO: 138) | | | | |
| | hzL4.3L VH | | | | | |
| | (SEQ ID NO: 97) | | | | | |
| hzL4L-5 | hzL4L VH G27Y, | hzL4L VL | 237 | 99 | Good | |
| | V67I, T68S, S70T, | (SEQ ID NO: 138) | | | | |
| | V71R | | | | | |
| | hzL4.4L VH | | | | | |
| | (SEQ ID NO: 98) | | | | | |
| hzL4L-6 | hzL4L VH G27Y, | hzL4L VL | 620 | 82 | Good | |
| | S30T, S70T, V71R | (SEQ ID NO: 138) | | | | |
| | hzL4.5L VH | | | | | |
| | (SEQ ID NO: 99) | | | | | |
| hzL4L-7 | hzL4L VH G27Y, | hzL4L VL | 407 | 97 | Good | 2.1 |
| | S70T, V71R | (SEQ ID NO: 138) | | | | |
| | hzL4.6L VH | | | | | |
| | (SEQ ID NO: 100) | | | | | |
| hzL4L-8 | hzL4L VH G27Y, | hzL4L VL | 316 | 99 | Poor | |
| | S30T, V67I, T68S | (SEQ ID NO: 138) | | | | |
| | hzL4.7L VH | | | | | |
| | (SEQ ID NO: 101) | | | | | |
| hzL4L-9 | hzL4L VH G27Y, | hzL4L VL | 291 | 98 | Poor | |
| | V67I, T68S | (SEQ ID NO: 138) | | | | |
| | hzL4.8L VH | | | | | |
| | (SEQ ID NO: 102) | | | | | |
| hzL4L-10 | hzL4L VH G27Y, | hzL4L VL D56S | 158 | 91 | Good | |
| | S30T, V67I, T68S, | hzL4.1L VL | | | | |
| | S70T, V71R | (SEQ ID NO: 139) | | | | |
| | hzL4.2L VH | | | | | |
| | (SEQ ID NO: 96) | | | | | |
| hzL4L-11 | hzL4L VH G27Y, | hzL4L VL D56E | 282 | 96 | Good | 2.0 |
| | S30T, V67I, T68S, | hzL4.2L VL | | | | |
| | S70T, V71R | (SEQ ID NO: 140) | | | | |
| | hzL4.2L VH | | | | | |
| | (SEQ ID NO: 96) | | | | | |
| hzL4L-12 | hzL4L VH G27Y, | hzL4L VL G57A | ND | ND | ND | ND |
| | S30T, V67I, T68S, | hzL4.3L VL | | | | |
| | S70T, V71R | (SEQ ID NO: 141) | | | | |
| | hzL4.2L VH | | | | | |
| | (SEQ ID NO: 96) | | | | | |
| hzL4L-13 | hzL4L VH G27Y, | hzL4L VL D56E | 429 | 99 | ND | 1.3 |
| | V67I, T68S, S70T, | hzL4.2L VL | | | | |
| | V71R | (SEQ ID NO: 140) | | | | |
| | hzL4.4L VH | | | | | |
| | (SEQ ID NO: 98) | | | | | |
| hzL4L-14 | hzL4L VH G27Y, | hzL4L VL D56E | 659 | 97 | ND | 1.4 |
| | S70T, V71R | hzL4.2L VL | | | | |
| | hzL4.6L VH | (SEQ ID NO: 140) | | | | |
| | (SEQ ID NO: 100) | | | | | |

### mAb H8K Humanization

Germline VH with JH6 was used for CDR grafting the variable heavy chain (SEQ ID NO:41) and germlines VL with JK4 were used for CDR grafting the variable light chain (SEQ ID NO: 129). CDR grafting was done using Kabat defined CDRs to generate hzH8K VH (SEQ ID NO: 103) and hzH8K VL (SEQ ID NO: 142). Several variants containing mouse framework back mutations were generated (SEQ ID NOS: 104-112), and the sequences determined using a 3D structural model for potential influence of residues on the CDR structure. Variable heavy region sequences were cloned into a vector containing a signal peptide sequence and IgG1 constant region. Variable light regions were cloned into a vector containing a signal peptide sequence and kappa constant region.

Ten humanized heavy chain were individually co-transfected with one humanized light chain into the ExpiCHO™ expression system in a 30 mL culture, using the parental chimeric antibody, H8K, as a benchmark. The supernatant was purified and further characterized as shown in Table 8.

TABLE 8
Humanized H8K Clones

| | | | Titer | % | HepG2 |
|---|---|---|---|---|---|
| Name | VH (IgG1) | VL (kappa) | mg/L | POI | EC₅₀ |
| H8K-C | H8K VH | H8K VL | 364 | 99 | 3.1 |
| | (SEQ ID NO: 41) | (SEQ ID NO: 129) | | | |
| hzH8K-1 | hzH8K VH | hzH8K VL | 435 | 99 | 3.4 |
| | (SEQ ID NO: 103) | (SEQ ID NO: 142) | | | |
| hzH8K-2 | hzH8K VH S30T | hzH8K VL | 438 | 99 | 2.5 |
| | hzH8.1K VH | (SEQ ID NO: 142) | | | |
| | (SEQ ID NO: 104) | | | | |
| hzH8K-3 | hzH8K VH G27F, | hzH8K VL | 442 | 99 | 3.2 |
| | S30T | (SEQ ID NO: 142) | | | |
| | hzH8.2K VH | | | | |
| | (SEQ ID NO: 105) | | | | |
| hzH8K-4 | hzH8K VH V67I, | hzH8K VL | 373 | 96 | 2.2 |
| | T68S, S70T, V71R | (SEQ ID NO: 142) | | | |
| | hzH8.3K VH | | | | |
| | (SEQ ID NO: 106) | | | | |
| hzH8K-5 | hzH8K VH S30T, | hzH8K VL | 421 | 94 | 3.6 |
| | V67I, T68S, S70T, | (SEQ ID NO: 142) | | | |
| | V71R | | | | |
| | hzH8.4K | | | | |
| | (SEQ ID NO: 107) | | | | |
| hzH8K-6 | hzH8K VH G27F, | hzH8K VL | 318 | 94 | 4.9 |
| | S30T, V67I, T68S, | (SEQ ID NO: 142) | | | |
| | S70T, V71R | | | | |
| | hzH8.5K | | | | |
| | (SEQ ID NO: 108) | | | | |
| hzH8K-7 | hzH8K VH S30T, | hzH8K VL | 617 | 98 | 3.1 |
| | V67I, T68S | (SEQ ID NO: 142) | | | |
| | hzH8.6K | | | | |
| | (SEQ ID NO: 109) | | | | |
| hzH8K-8 | hzH8K VH S30T, | hzH8K VL | 587 | 92 | 3.5 |
| | S70T, V71R | (SEQ ID NO: 142) | | | |
| | hzH8.7K | | | | |
| | (SEQ ID NO: 110) | | | | |
| hzH8K-9 | hzH8K VH V67I, | hzH8K VL | 440 | 97 | 2.1 |
| | T68S | (SEQ ID NO: 142) | | | |
| | hzH8.8K | | | | |
| | (SEQ ID NO: 111) | | | | |
| hzH8K-10 | hzH8K VH S70T, | hzH8K VL | 744 | 96 | 2.2 |
| | V71R | (SEQ ID NO: 142) | | | |
| | hzH8.9K | | | | |
| | (SEQ ID NO: 112) | | | | |

### J4F Humanization

Germline VH1-46 with JH6 was used for CDR grafting the variable heavy chain (SEQ ID NO:42) and germline VL with JK4 was used for CDR grafting the variable light chain (SEQ ID NO: 130). CDR grafting was done using Kabat defined CDRs to generate hzJ4F VH (SEQ ID NO: 113) and hzJ4F VL (SEQ ID NO: 143). Several variants containing mouse framework back mutations were generated (SEQ ID NOS: 114-125 for VH and SEQ ID NOS: 144-149 for VL), and the sequences determined using a 3D structural model for potential influence of residues on the CDR structure. Variable heavy region sequences were cloned into a vector containing a signal peptide sequence and IgG1 constant region. Variable light regions were cloned into a vector containing a signal peptide sequence and kappa constant region.

Thirteen humanized heavy chains were each individually co-transfected with each of seven different humanized light chains into the ExpiCHO™ expression system in a 30 mL culture, using the parental chimeric antibody, J4F, as a benchmark. Humanized variants hzJ4F-1 through hzJ4F-20 showed reduced binding compared to the parent chimeric mAbJ4F (see Table 9). Therefore, additional humanized variable region sequences were designed to screen for hzJ4F heavy chain and light chain combinations (hzJ4F-21-hzJ4F-58) with binding similar to or better than the chimeric parent mAb J4F.

TABLE 9
Humanized J4F Clones

| Name | VH (IgG1) | VL (kappa) | Binding |
|---|---|---|---|
| J4F | J4F VH | J4F VL | ++++ |
| | (SEQ ID NO: 42) | (SEQ ID NO: 130) | |
| hzJ4F-1 | hzJ4F VH | hzJ4F VL | ++++ |
| | (SEQ ID NO: 113) | (SEQ ID NO: 143) | |
| hzJ4F-2 | hzJ4F VH R66K, | hzJ4F VL | ++ |
| | V67A, M69L, R71V | (SEQ ID NO: 143) | |
| | hzJ4.8F VH | | |
| | (SEQ ID NO: 121) | | |
| hzJ4F-3 | hzJ4F VH R66K, | hzJ4F VL | ++ |
| | V67A | (SEQ ID NO: 143) | |
| | hzJ4.5F VH | | |
| | (SEQ ID NO: 118) | | |
| hzJ4F-4 | hzJ4F VH M69L, | hzJ4F VL | + |
| | R71V | (SEQ ID NO: 143) | |
| | hzJ4.9F VH | | |
| | (SEQ ID NO: 122) | | |
| hzJ4F-5 | hzJ4F VH R71V | hzJ4F VL | −/+ |
| | hzJ4.10F VH | (SEQ ID NO: 143) | |
| | (SEQ ID NO: 123) | | |
| hzJ4F-6 | hzJ4F VH | hzJ4F VL Q3V | ++ |
| | (SEQ ID NO: 113) | hzJ4.1F VL | |
| | | (SEQ ID NO: 144) | |
| hzJ4F-7 | hzJ4F VH R66K, | hzJ4F VL Q3V | ++ |
| | V67A, M69L, R71V | hzJ4.1F VL | |
| | hzJ4.8F VH | (SEQ ID NO: 144) | |
| | (SEQ ID NO: 121) | | |
| hzJ4F-8 | hzJ4F VH R66K, | hzJ4F VL Q3V | ++ |
| | V67A | hzJ4.1F VL | |
| | hzJ4.5F VH | (SEQ ID NO: 144) | |
| | (SEQ ID NO: 118) | | |
| hzJ4F-9 | hzJ4F VH M69L, | hzJ4F VL Q3V | + |
| | R71V | hzJ4.1F VL | |
| | hzJ4.9F VH | (SEQ ID NO: 144) | |
| | (SEQ ID NO: 122) | | |
| hzJ4F-10 | hzJ4F VH R71V | hzJ4F VL Q3V | ++ |
| | hzJ4.10F VH | hzJ4.1F VL | |
| | (SEQ ID NO: 123) | (SEQ ID NO: 144) | |
| hzJ4F-11 | hzJ4F VH | hzJ4F VL L46A | ++ |
| | (SEQ ID NO: 113) | hzJ4.3F VL | |
| | | (SEQ ID NO: 146) | |
| hzJ4F-12 | hzJ4F VH R66K, | hzJ4F VL L46A | ++ |
| | V67A, M69L, R71V | hzJ4.3F VL | |
| | hzJ4.8F VH | (SEQ ID NO: 146) | |
| | (SEQ ID NO: 121) | | |
| hzJ4F-13 | hzJ4F VH R66K, | hzJ4F VL L46A | ++ |
| | V67A | hzJ4.3F VL | |
| | hzJ4.5F VH | (SEQ ID NO: 146) | |
| | (SEQ ID NO: 118) | | |
| hzJ4F-14 | hzJ4F VH M69L, | hzJ4F VL L46A | ++ |
| | R71V | hzJ4.3F VL | |
| | hzJ4.9F VH | (SEQ ID NO: 146) | |
| | (SEQ ID NO: 122) | | |
| hzJ4F-15 | hzJ4F VH R71V | hzJ4F VKL L46A | ++ |
| | hzJ4.10F VH | hzJ4.3F VL | |
| | (SEQ ID NO: 123) | (SEQ ID NO: 146) | |
| hzJ4F-16 | hzJ4F VH | hzJ4F VL Q3V L46A | ++ |
| | (SEQ ID NO: 113) | hzJ4.2F VL | |
| | | (SEQ ID NO: 145) | |
| hzJ4F-17 | hzJ4F VH R66K, | hzJ4F VL Q3V L46A | ++ |
| | V67A, M69L, R71V | hzJ4.2F VL | |
| | hzJ4.8F VH | (SEQ ID NO: 145) | |
| | (SEQ ID NO: 121) | | |
| hzJ4F-18 | hzJ4F VH R66K, | hzJ4F VL Q3V L46A | ++ |
| | V67A | hzJ4.2F VL | |
| | hzJ4.5F VH | (SEQ ID NO: 145) | |
| | (SEQ ID NO: 118) | | |
| hzJ4F-19 | hzJ4F VH M69L, | hzJ4F VL Q3V L46A | ++ |
| | R71V | hzJ4.2F VL | |
| | hzJ4.9F VH | (SEQ ID NO: 145) | |
| | (SEQ ID NO: 122) | | |
| hzJ4F-20 | hzJ4F VH R71V | hzJ4F VL Q3V L46A | ++ |
| | hzJ4.10F VH | hzJ4.2F VL | |
| | (SEQ ID NO: 123) | (SEQ ID NO: 145) | |
| hzJ4F-21 | hzJ4F VH V37I | hzJ4F VL L46A | |
| | hzJ4.1F VH | hzJ4.3F VL | |
| | (SEQ ID NO: 114) | (SEQ ID NO: 146) | |
| hzJ4F-22 | hzJF4 VH V37I, | hzJ4F VL L46A | |
| | R38K, M48I | hzJ4.3F VL | |
| | hzJ4.2F VH | (SEQ ID NO: 146) | |
| | (SEQ ID NO: 115) | | |
| hzJ4F-23 | hzJ4F VH V37I, | hzJ4F VL L46A | |
| | R66K, V67A | hzJ4.3F VL | |
| | hzJ4.3F VH | (SEQ ID NO: 146) | |
| | (SEQ ID NO: 116) | | |
| hzJ4F-24 | hzJ4F VH V37I, | hzJ4F VL L46A | |
| | R38K, M48I, R66K, | hzJ4.3F VL | |
| | V67A | (SEQ ID NO: 146) | |
| | hzJ4.4F VH | | |
| | (SEQ ID NO: 117) | | |
| hzJ4F-25 | hzJ4F VH V78A | hzJ4F VL L46A | |
| | hzJ4.11F VH | hzJ4.3F VL | |
| | (SEQ ID NO: 124) | (SEQ ID NO: 146) | |
| hzJ4F-26 | hzJ4F VH V78A, | hzJ4F VL L46A | |
| | M80I, E81Q | hzJ4.3F VL | |
| | hzJ4.12F VH | (SEQ ID NO: 146) | |
| | (SEQ ID NO: 125) | | |
| hzJ4F-27 | hzJ4F VH R66K, | hzJ4F VL L46A | |
| | V67A, V78A | hzJ4.3F VL | |
| | hzJ4.6F VH | (SEQ ID NO: 146) | |
| | (SEQ ID NO: 119) | | |
| hzJ4F-28 | hzJ4F VH R66K, | hzJ4F VL L46A | |
| | V67A, V78A, M80I, | hzJ4.3F VL | |
| | E81Q | (SEQ ID NO: 146) | |
| | hzJ4.7F VH | | |
| | (SEQ ID NO: 120) | | |
| hzJ4F-29 | hzJ4F VH | hzJ4F VL L46A, | |
| | (SEQ ID NO: 113) | L47V | |
| | | hzJ4.4F VL | |
| | | (SEQ ID NO: 147) | |
| hzJ4F-30 | hz9C5.1 VH R66K, | hzJ4F VL L46A, | |
| | V67A | L47V | |
| | hzJ4.5F VH | hzJ4.4F VL | |
| | (SEQ ID NO: 118) | (SEQ ID NO: 147) | |
| hzJ4F-31 | hzJ4F VH V37I | hzJ4F VL L46A, | |
| | hzJ4.1F VH | L47V | |
| | (SEQ ID NO: 114) | hzJ4.4F VL | |
| | | (SEQ ID NO: 147) | |
| hzJ4F-32 | hzJF4 VH V37I, | hzJ4F VL L46A, | |
| | R38K, M48I | L47V | |
| | hzJ4.2F VH | hzJ4.4F VL | |
| | (SEQ ID NO: 115) | (SEQ ID NO: 147) | |
| hzJ4F-33 | hzJ4F VH V37I, | hzJ4F VL L46A, | |
| | R66K, V67A | L47V | |
| | hzJ4.3F VH | hzJ4.4F VL | |
| | (SEQ ID NO: 116) | (SEQ ID NO: 147) | |
| hzJ4F-34 | hzJ4F VH V37I, | hzJ4F VL L46A, | |
| | R38K, M48I, R66K, | L47V | |
| | V67A | hzJ4.4F VL | |
| | hzJ4.4F VH | (SEQ ID NO: 147) | |
| | (SEQ ID NO: 117) | | |
| hzJ4F-35 | hzJ4F VH V78A | hzJ4F VL L46A, | |
| | hzJ4.11F VH | L47V | |
| | (SEQ ID NO: 124) | hzJ4.4F VL | |
| | | (SEQ ID NO: 147) | |
| hzJ4F-36 | hzJ4F VH V78A, | hzJ4F VL L46A, | |
| | M80I, E81Q | L47V | |
| | hzJ4.12F VH | hzJ4.4F VL | |
| | (SEQ ID NO: 125) | (SEQ ID NO: 147) | |
| hzJ4F-37 | hzJ4F VH R66K, | hzJ4F VL L46A, | |
| | V67A, V78A | L47V | |
| | hzJ4.6F VH | hzJ4.4F VL | |
| | (SEQ ID NO: 119) | (SEQ ID NO: 147) | |
| hzJ4F-38 | hzJ4F VH R66K, | hzJ4F VL L46A, | |
| | V67A, V78A, M80I, | L47V | |
| | E81Q | hzJ4.4F VL | |
| | hzJ4.7F VH | (SEQ ID NO: 147) | |
| | (SEQ ID NO: 120) | | |
| hzJ4F-39 | hzJ4F VH | hzJ4F VL L46A, | |
| | (SEQ ID NO: 113) | L47V, F62L | |
| | | hzJ4.5F VL | |
| | | (SEQ ID NO: 148) | |
| hzJ4F-40 | hz9C5.1 VH R66K, | hzJ4F VL L46A, | |
| | V67A | L47V, F62L | |
| | hzJ4.5F VH | hzJ4.5F VL | |
| | (SEQ ID NO: 118) | (SEQ ID NO: 148) | |
| hzJ4F-41 | hzJ4F VH V37I | hzJ4F VL L46A, | |
| | hzJ4.1F VH | L47V, F62L | |
| | (SEQ ID NO: 114) | hzJ4.5F VL | |
| | | (SEQ ID NO: 148) | |
| hzJ4F-42 | hzJF4 VH V37I, | hzJ4F VL L46A, | |
| | R38K, M48I | L47V, F62L | |
| | hzJ4.2F VH | hzJ4.5F VL | |
| | (SEQ ID NO: 115) | (SEQ ID NO: 148) | |
| hzJ4F-43 | hzJ4F VH V37I, | hzJ4F VL L46A, | |
| | R66K, V67A | L47V, F62L | |
| | hzJ4.3F VH | hzJ4.5F VL | |
| | (SEQ ID NO: 116) | (SEQ ID NO: 148) | |
| hzJ4F-44 | hzJ4F VH V37I, | hzJ4F VL L46A, | |
| | R38K, M48I, R66K, | L47V, F62L | |
| | V67A | hzJ4.5F VL | |
| | hzJ4.4F VH | (SEQ ID NO: 148) | |
| | (SEQ ID NO: 117) | | |
| hzJ4F-45 | hzJ4F VH V78A | hzJ4F VL L46A, | |
| | hzJ4.11F VH | L47V, F62L | |
| | (SEQ ID NO:124) | hzJ4.5F VL | |
| | | (SEQ ID NO: 148) | |
| hzJ4F-46 | hzJ4F VH V78A, | hzJ4F VL L46A, | |
| | M80I, E81Q | L47V, F62L | |
| | hzJ4.12F VH | hzJ4.5F VL | |
| | (SEQ ID NO: 125) | (SEQ ID NO: 148) | |
| hzJ4F-47 | hzJ4F VH R66K, | hzJ4F VL L46A, | |
| | V67A, V78A | L47V, F62L | |
| | hzJ4.6F VH | hzJ4.5F VL | |
| | (SEQ ID NO: 119) | (SEQ ID NO: 148) | |
| hzJ4F-48 | hzJ4F VH R66K, | hzJ4F VL L46A, | |
| | V67A, V78A, M80I, | L47V, F62L | |
| | E81Q | hzJ4.5F VL | |
| | hzJ4.7F VH | (SEQ ID NO: 148) | |
| | (SEQ ID NO: 120) | | |
| hzJ4F-49 | hzJ4F VH | hzJ4F VL L46A, | |
| | (SEQ ID NO: 113) | L47V, S60D, F62L, | |
| | | S63T | |
| | | hzJ4.6F VL | |
| | | (SEQ ID NO: 149) | |
| hzJ4F-50 | hzJ4F VH R66K, | hzJ4F VL L46A, | |
| | V67A | L47V, S60D, F62L, | |
| | hzJ4.5F VH | S63T | |
| | (SEQ ID NO: 118) | hzJ4.6F VL | |
| | | (SEQ ID NO: 149) | |
| hzJ4F-51 | hzJ4F VH V37I | hzJ4F VL L46A, | |
| | hzJ4.1F VH | L47V, S60D, F62L, | |
| | (SEQ ID NO: 114) | S63T | |
| | | hzJ4.6F VL | |
| | | (SEQ ID NO: 149) | |
| hzJ4F-52 | hzJF4 VH V37I, | hzJ4F VL L46A, | |
| | R38K, M48I | L47V, S60D, F62L, | |
| | hzJ4.2F VH | S63T | |
| | (SEQ ID NO: 115) | hzJ4.6F VL | |
| | | (SEQ ID NO: 149) | |
| hzJ4F-53 | hzJ4F VH V37I, | hzJ4F VL L46A, | |
| | R66K, V67A | L47V, S60D, F62L, | |
| | hzJ4.3F VH | S63T | |
| | (SEQ ID NO: 116) | hzJ4.6F VL | |
| | | (SEQ ID NO: 149) | |
| hzJ4F-54 | hzJ4F VH V37I, | hzJ4F VL L46A, | |
| | R38K, M48I, R66K, | L47V, S60D, F62L, | |
| | V67A | S63T | |
| | hzJ4.4F VH | hzJ4.6F VL | |
| | (SEQ ID NO: 117) | (SEQ ID NO: 149) | |
| hzJ4F-55 | hzJ4F VH V78A | hzJ4F VL L46A, | |
| | hzJ4.11F VH | L47V, S60D, F62L, | |
| | (SEQ ID NO: 124) | S63T | |
| | | hzJ4.6F VL | |
| | | (SEQ ID NO: 149) | |
| hzJ4F-56 | hzJ4F VH V78A, | hzJ4F VL L46A, | |
| | M80I, E81Q | L47V, S60D, F62L, | |
| | hzJ4.12F VH | S63T | |
| | (SEQ ID NO: 125) | hzJ4.6F VL | |
| | | (SEQ ID NO: 149) | |
| hzJ4F-57 | hzJ4F VH R66K, | hzJ4F VL L46A, | |
| | V67A, V78A | L47V, S60D, F62L, | |
| | hzJ4.6F VH | S63T | |
| | (SEQ ID NO: 119) | hzJ4.6F VL | |
| | | (SEQ ID NO: 149) | |
| hzJ4F-58 | hzJ4F VH R66K, | hzJ4F VL L46A, | |
| | V67A, V78A, M80I, | L47V, S60D, F62L, | |
| | E81Q | S63T | |
| | hzJ4.7F VH | hzJ4.6F VL | |
| | (SEQ ID NO: 120) | (SEQ ID NO: 149) | |

### Example 8

### Production of Exemplary ASGRI1 Antibodies with Fc Domain Mutations

4A2 Fc variants were generated in similar manner as described in Example 7. The human IgG1 null Fc domain comprised the following mutations: L234A, L235A, G237A, and K322A based on EU numbering. The IgG1 SELF domain comprised the following mutations: S267E and L328F based on EU numbering. The LF variant contained the L328F mutation. Both the IgG1 SELF and LF constructs also contained the K322A mutation to disable CDC function without affecting FcgR binding. The following constructs were generated:

a. 4A2 IgG1 SELF K322A

b. 4A2 IgG1 LF K322A

c. 4A2 IgG1 null

d. 4A2 IgG1 null SELF

e. 4A2 IgG1 null L328F

### Example 9

### Production of Exemplary Cysteine Engineered ASGR1 Antibodies

Two cysteine engineered variants (4A2 IgG1 A114C-HC and 4A2 IgG1 V205C-LC) were generated and the protein was produced in a similar manner as in Example 8. 4A2 IgG1 A114C-HC contained the Alanine to Cysteine mutation on the heavy chain at position 114 based on EU numbering. 4A2 V205C-LC contained the Valine to Cysteine mutation on the light chain at position 205. Binding data confirmed that the mutation on the Fc variants and the cysteine engineered variants does not impact binding to ASGR1.

### Example 10

### Characterization of Cysteine Engineered ASGR1 4A2 Exemplary Antibodies Using ELISA FcgR Binding Assay

ELISA plates were coated with 50 ul of various recombinant Fcg receptors (human and Marmota) at 0.1 ug/ml diluted in 1×PBS. Following overnight incubation, the plates were washed with the wash buffer (1×PBS+0.05% Tween 20), blocked with the blocking buffer (1% nonfat milk in PBS) and incubated for 1 hour at room temperature. The ELISA plates were then washed with the wash buffer and ASGR1 test samples (naked mAb or cysteine engineered Ab) were added from a concentration of 100 ug/ml diluted 3 fold. The plates were then incubated for 1 hour at room temperature and washed again 3× with wash buffer. 100 ul of the HRP goat anti-human Fc antibody diluted 1:1000 fold was next added into each well and incubated for another hour. The plates were washed 4× with the wash buffer. 50 ul of the TMB ELISA substrate was next added for color development and the reaction stopped by adding 50 ul of 2M sulfuric acid. The plates were then read on a plate reader at 450 nm wavelength.

When 4A2 IgG1 and the cysteine engineered variants were compared across different Human and Marmota Fcg receptors (Human FcgR2a, Human FcgR2b, Marmota FcgR1, Marmota FcgR2b, and Marmota FcgR3a), the binding affinities were all similar indicating that the cysteine mutation introduced for the cysteine engineered variants does not affect FcgR binding.

### Example 11

### Characterization of ASGR1 4A2 Conjugates Binding to Human FcgR1 Receptors

4A2 IgG1 and conjugates are analyzed for human FcγR1 interaction analysis using an Octet Red 96m instrument. The antibody or conjugates are immobilized on anti-human Fc biosensors and incubated with varying concentration of monomeric FcγR1 ranging from 1.2 nM to 1 μM in PBS. The experiments are performed using five steps: (1) baseline acquisition (60 s); (2) antibody or conjugate loading onto anti-human Fc biosensor (120 s); (3) second baseline acquisition (60 s); (4) association of interacting protein for kₒₙ measurement (120 s); and (5) dissociation of interacting FcγR1 for k_{off} measurement (300 s). The interacting monomeric FcγR1 is used at 5-6 concentrations of a 3-fold concentration series. The data are analyzed using Octet Data Analysis Software 9.0 (ForteBio)™ and fit to the 1:1 binding model. Equilibrium dissociation constants (K_{D}) are calculated by the ratio of kₒₙ to k_{off}.

### Example 12

### Determination of Molar Ratio

Method A. This example illustrates one method by which the molar ratio is determined. One microgram of conjugate is injected into an LC/MS such as an Agilent 6550 iFunnel Q-TOF equipped with an Agilent Dual Jet Stream ESI source coupled with Agilent 1290 Infinity UHPLC system. Raw data is obtained and is deconvoluted with software such as Agilent MassHunter Qualitative Analysis Software with BioConfirm using the Maximum Entropy deconvolution algorithm. The average mass of intact conjugates is calculated by the software, which can use top peak height at 25% for the calculation. This data is then imported into another program to calculate the molar ratio of the TGFβR1 inhibitor:conjugate, such as Agilent molar ratio calculator.

Method B. Another method for determination of molar ratio is as follows. First, 10 μL of a 5 mg/mL solution of a conjugate is injected into an HPLC system set-up with a TOSOH TSKgel Butyl-NPR™ hydrophobic interaction chromatography (HIC) column (2.5 μM particle size, 4.6 mm×35 mm) attached. Then, over the course of 18 minutes, a method is run in which the mobile phase gradient is run from 100% mobile phase A to 100% mobile phase B over the course of 12 minutes, followed by a six minute re-equilibration at 100% mobile phase A. The flow rate is 0.8 mL/min and the detector is set at 280 nM. Mobile phase A is 1.5 M ammonium sulfate, 25 mM sodium phosphate (pH 7). Mobile phase B is 25% isopropanol in 25 mM sodium phosphate (pH 7). Post-run, the chromatogram is integrated and the molar ratio is determined by summing the weighted peak area.

### Example 13

### General Process for Identifying Liver-Specific Antigens

A compendium of normal tissue expression data, for example an RNA-Seq transcriptomic database such as GTEx, is used to create segregated sets of samples by tissue type. This database is then partitioned into test (tissues with desired expression) and control (tissues with undesired expression) subsets, in this case liver versus all other tissues, and an analysis of variance test such as the Kruskal-Wallis one-way analysis of variance is performed to identify genes that are significantly differentially expressed between the two sample sets. Because the resulting list of genes may include examples which while over-expressed in the test group when compared to the normal group as a whole may still have higher expression in individual tissue types than desired, a second filtering step is applied wherein those genes whose average tissue-specific expression for any specific tissue in the control tissue set is above a desired cutoff are removed from further analysis. If desired, a further filtering step can be applied to sort and restrict genes returned based on absolute minimum average expression in the test set, absolute maximum average expression in the control set and/or the ratio of the test-to-control expression. From this resulting list, a final filtering step is performed as desired to include or exclude genes based on the cellular localization of the protein product. For example, it may be desired to include only proteins expressed on the cell surface, or of those single-pass surface proteins. When selecting the final list, additional consideration is also given to expression patterns of the antigen in one or more specific diseases, or in liver cell subsets. For example, in the case of liver cell subsets the expression of the antigen in question would be compared in available liver cell subsets (such as hepatocytes, Kupffer cells, or hepatic stellate cells) and antigens removed, retained, or prioritized in consideration depending on these expression patterns. Where possible, protein-level confirmation of expression of candidate genes is performed by examining available immunohistochemistry data such as that found in the Human Protein Atlas.

### Example 14

### Determination of K_{d} Values

K_{d} is measured using surface plasmon resonance assays using a BIACORE®-2000 or a BIACORE®-3000 (BIAcore, Inc., Piscataway, N.J.) at 25° C. with immobilized antigen CM5 chips at ^{˜}10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/mL (^{˜}0.2 μM) before injection at a flow rate of 5 μL/minute to achieve approximately 10 response units (RU) of coupled protein. Following the injection of antigen, 1 M ethanolamine is injected to block unreacted groups. For kinetics measurements, two-fold serial dilutions of Fab or conjugate (0.78 nM to 500 nM) are injected in PBS with 0.05% polysorbate 20 (TWEEN-20™) surfactant (PBST) at 25° C. at a flow rate of approximately 25 L/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{d}) is calculated as the ratio k_{off}/kₒₙ. See, e.g., Chen et al., J. Mol. Biol. 293:865-881 (1999). If the on-rate exceeds 10⁶ M-1 s-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form or conjugate form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### Example 15

### Effect of Anti-ASGR1-ALK5 Inhibitor Conjugates on TGFb Production

Anti-ASGR1 antibodies conjugated to an ALK5 inhibitor were tested via a cell reporter assay. Briefly, HEK293 SBE-LUC reporter cells transfected to stably express full length human ASGR1 were seeded in 96 well plates at 40,000 cells/well in an assay media of minimum essential media containing 0.5% fetal bovine serum, 1% nonessential amino acids, 1% sodium pyruvate and 1% Pen/Strep. Anti-ASGR1-ALK5 inhibitor conjugates and controls were added to wells in a dose titration ranging from 5 μM to 0.064 nM to HEK293 SBE-LUC and ASGR1-HEK293 SBE-LUC cells. After incubating for 6 hours at 37° C. in a 5% CO₂ environment, human TGFβ 1 (PeproTech Inc.) was added to a final concentration of 1.6 ng/ml followed by an additional 18 hour incubation under the same conditions. Luciferase STEADY-GLO® reagent (Promega Corporation) was added at 70 μl/well and incubated with shaking for 10 minutes. Luciferase activity was determined by measuring luminescence with an ENVISION® Plate Reader (Perkin-Elmer Inc.). Data were fit with a four-parameter non-linear regression to calculate IC₅₀ values using Prism Software v7.04 (GraphPad Inc.).

Table 10 shows that each of the anti-ASGR1-ALK5 inhibitor conjugates could efficiently inhibit TGFβ1-mediated luciferase expression as compared to an unconjugated anti-ASGR antibody (mAb-A) alone. Furthermore, the ASGR-ALK5 inhibitor conjugates were more potent than the ALK5 inhibitor (Compound 60) alone.

TABLE 10
AASGR1-ALK5 Inhibitor Conjugate Inhibition
of TGFβ1-Mediated Luciferase Expression

| Test Article | IC₅₀ (nM) |
|---|---|
| Compound 60 | 13.4 |
| ASGR1-mAb-A | —* |
| ASGR1-mAb-A-(IVb.2-Cmpd 60) | 7.2 |
| ASGR1-mAb-B-(IVb.2-Cmpd 60) | 9.1 |
| G2D-(IVb.2-Cmpd 60) | 4.3 |
| J4F-(IVb.2-Cmpd 60) | 6.5 |
| ASGR1-mAb-C-(IVb.2-Cmpd 60) | 13.9 |
| L4L-(IVb.2-Cmpd 60) | 8.5 |
| H8K-(IVb.2-Cmpd 60) | 3.8 |

*— = No detectable activity

### Example 16

### TGFβ Reporter Assay

TGFβ/SMAD Signaling Pathway SBE reporter cell line was obtained from BPS Bioscience. Cells are passed, expanded, and stored in liquid nitrogen as per the supplier's instructions with the exception that growth media is changed to DMEM-C with Geneticin (DMEM supplemented with 10% fetal bovine serum, 1×NEAA, 1 mM Pyruvate, 2 mM glutamine, 50 μg/mL penicillin, 50 U/mL streptomycin and 400 μg/mL of Geneticin). The assay media is MEM supplemented with 0.5% fetal bovine serum, 1×NEAA, 1 mM Pyruvate, 50 μg/mL penicillin and 50 U/mL streptomycin.

Compounds are assayed to measure their activity as ALK5 inhibitors.

### Enzyme Inhibition Assay

ALK5 enzyme inhibition assays were performed by Reaction Biology Corp (Malvern, Pa.). 1 mg/mL of peptide substrate (casein) and 10 uM ATP were prepared in a mixture of fresh reaction buffer. The kinase was delivered into the substrate solution which was gently mixed. Compounds in 100% DMSO were added to the kinase reaction mixture by Acoustic technology (Echo550; nanoliter range) and the mixture was incubated for 20 min at room temperature. ³³P-ATP (Specific activity 10 uCi/uL) was added into the reaction mixture to initiate the reaction and the reaction mixture was incubated for 2 hours at room temperature. Radioactivity was detected by filter-binding method and kinase activity data were expressed as the percent remaining kinase activity in test samples compared to vehicle (dimethyl sulfoxide) reactions. IC₅₀ values and curve fits were obtained using Prism (GraphPad Software). Results are reported in Tables 11 and 12.

TABLE 11

| Compound | IC₅₀ |
|---|---|
| 1 | ++ |
| 2 | ++ |
| 3 | + |
| 4 | ++ |
| 5 | +++ |
| 6 | ++++ |
| 7 | ++ |
| 8 | + |
| 9 | ++ |
| 10 | ++++ |
| 11 | ++ |
| 12 | + |
| 13 | +++ |
| 14 | +++ |
| 15 | +++ |
| 16 | ++++ |
| 17 | +++ |
| 18 | ++++ |
| 19 | ++++ |
| 20 | + |
| 21 | ++++ |
| 22 | ++++ |
| 23 | +++ |
| 24 | ++++ |
| 25 | ++ |
| 26 | ++++ |
| 27 | +++ |
| 28 | ++++ |
| 29 | ++ |
| 30 | ++ |
| 31 | ++ |
| 32 | ++ |
| 33 | ++ |
| 34 | ++ |
| 35 | ++++ |
| 36 | +++ |
| 37 | +++ |
| 38 | ++++ |
| 39 | ++++ |
| 40 | ++ |
| 41 | +++ |
| 42 | ++ |
| 43 | ++ |
| 44 | ++ |
| 45 | +++ |
| 46 | +++ |
| 47 | ++++ |
| 48 | ++++ |
| 49 | ++ |
| 50 | ++ |
| 51 | ++ |
| 52 | +++ |
| 53 | ++++ |
| 54 | +++ |
| 55 | NT |
| 56 | NT |
| 57 | NT |
| 58 | NT |
| 59 | ++++ |
| 60 | ++++ |
| 61 | ++++ |
| 62 | ++ |
| 63 | ++ |
| 64 | ++ |
| 65 | ++ |
| 66 | ++ |
| 67 | +++ |
| 68 | ++++ |
| 69 | ++ |
| 70 | NT |
| 71 | ++++ |
| 72 | NT |
| 73 | NT |
| 74 | NT |
| 75 | NT |
| 76 | NT |
| 77 | NT |
| 78 | NT |
| 79 | NT |
| 80 | NT |
| 81 | ++++ |
| 82 | ++++ |
| 83 | +++ |
| 84 | ++ |
| 85 | +++ |
| 86 | ++++ |
| 87 | ++++ |
| 88 | ++++ |
| 89 | ++++ |
| 90 | +++ |
| 91 | ++ |

Table 11 Key: Compounds having an IC₅₀ value between 0.1 nM and 50 nM are denoted as ++++, 50 nM and 100 nM as +++, 100 nM and 1000 nM as ++, and 1000 nM to 10,000 nM as +.

TABLE 12

| Compound | IC₅₀ |
|---|---|
| 1.1 | ++ |
| 1.2 | ++ |
| 1.3 | ++ |
| 1.4 | ++ |
| 1.5 | ++ |
| 1.6 | ++ |
| 1.7 | + |
| 1.8 | + |
| 1.9 | + |
| 1.10 | + |
| 1.11 | ++ |
| 1.12 | ++ |
| 1.13 | ++ |
| 1.14 | ++ |
| 1.15 | ++ |
| 1.16 | ++ |
| 1.19 | + |
| 1.20 | ++ |
| 1.21 | ++ |
| 1.22 | + |
| 1.23 | +++ |
| 1.24 | ++ |
| 1.25 | ++ |
| 1.26 | ++ |
| 1.27 | ++ |
| 1.30 | ++ |
| 1.31 | ++ |
| 1.32 | +++ |
| 1.33 | ++ |
| 1.34 | ++ |
| 1.35 | + |
| 1.32 | +++ |
| 1.33 | ++ |
| 1.40 | ++ |
| 1.41 | ++ |
| 1.42 | ++ |
| 1.43 | +++ |
| 1.44 | ++ |
| 1.45 | ++ |
| 1.46 | ++ |
| 1.48 | + |
| 1.51 | + |
| 1.52 | + |
| 1.55 | + |
| 1.56 | ++ |
| 1.57 | +++ |
| 1.58 | ++ |
| 1.59 | +++ |
| 1.60 | ++ |
| 1.61 | ++ |

Table 12 Key: Compounds having an IC₅₀ value between 0.1 nM and 10 nM are denoted as ++++, 10 nM and 100 nM as +++, 100 nM and 1000 nM as ++, and 1000 nM to 10,000 nM as +.

### TGFb Reporter Assay

The HEK293 SBE-LUC reporter cells from Example 15 were grown in DMEM-C with Geneticin (DMEM supplemented with 0% fetal bovine serum, 1×NEAA, 1 mM Pyruvate, 2 mM glutamine, 50 μg/mL penicillin, 50 U/mL streptomycin and 400 ug/mL of Geneticin). The assay media was MEM supplemented with 0.5% fetal bovine serum, 1×NEAA, 1 mM Pyruvate, 50 μg/mL penicillin and 50 U/mL streptomycin.

The HEK293 SBE-LUC reporter cells were harvested from the tissue culture flasks by incubation in small quantity of Versene at room temperature for three to five minutes after the media in the flask is removed and cells rinsed with PBS. Cells were counted and diluted in the assay media at ˜0.8×10⁶ cells/mL then 50 uL/well were added to 96-well assay plate. Test samples (at desired concentrations diluted in assay media) were added to assay plate containing the 50 uL/well of cells (or media only), 50 uL per well, and incubated for 5-6 hours at 37° C. in a 5% CO₂ humidified incubator. After that time, 15 uL of TGFb diluted to 12.5 ng/mL in the assay media was added to the plate. Controls included TGFb titration (from 25 to 0 ng/mL) without inhibitors, and media only (without cells, inhibitor or TGFb). Plates were incubated at 37° C. in a 5% CO₂ humidified incubator for 18h. Luciferase substrate solution was subsequently added at 75 uL per well, incubated in dark with shaking at room temperature for 10 min, and luminescence was measured using a luminometer. Results are shown in Table 13.

TABLE 13

| Compound | EC₅₀ |
|---|---|
| 1 | ++++ |
| 2 | +++ |
| 3 | +++ |
| 4 | ++ |
| 5 | +++ |
| 6 | ++++ |
| 7 | ++++ |
| 8 | ++ |
| 9 | +++ |
| 10 | ++++ |
| 11 | +++ |
| 12 | ++ |
| 13 | ++++ |
| 14 | +++ |
| 15 | ++ |
| 16 | ++ |
| 17 | +++ |
| 18 | ++++ |
| 19 | ++++ |
| 20 | ++ |
| 21 | ++++ |
| 22 | ++++ |
| 23 | ++++ |
| 24 | ++++ |
| 25 | ++++ |
| 26 | ++++ |
| 27 | ++++ |
| 28 | ++++ |
| 29 | ++ |
| 30 | ++ |
| 31 | ++ |
| 32 | ++ |
| 33 | ++ |
| 34 | ++ |
| 35 | ++++ |
| 36 | +++ |
| 37 | +++ |
| 38 | ++++ |
| 39 | +++ |
| 40 | +++ |
| 41 | ++++ |
| 42 | +++ |
| 43 | ++ |
| 44 | +++ |
| 45 | ++++ |
| 46 | ++++ |
| 47 | ++++ |
| 48 | +++ |
| 49 | +++ |
| 50 | +++ |
| 51 | ++ |
| 52 | ++++ |
| 53 | ++++ |
| 54 | ++++ |
| 55 | + |
| 56 | +++ |
| 57 | +++ |
| 58 | +++ |
| 59 | ++++ |
| 60 | ++++ |
| 61 | ++++ |
| 62 | ++++ |
| 63 | ++++ |
| 64 | ++++ |
| 65 | ++++ |
| 66 | ++++ |
| 67 | ++++ |
| 68 | +++ |
| 69 | ++ |
| 70 | NT |
| 71 | ++ |
| 72 | +++ |
| 73 | ++ |
| 74 | ++ |
| 75 | ++ |
| 76 | +++ |
| 77 | ++++ |
| 78 | ++++ |
| 79 | ++ |
| 80 | ++ |
| 81 | +++ |
| 82 | ++++ |
| 83 | ++++ |
| 84 | ++++ |
| 85 | ++++ |
| 86 | ++++ |
| 87 | ++++ |
| 88 | ++++ |
| 89 | NT |
| 90 | ++++ |
| 91 | +++ |
| 1.1 | ++ |
| 1.2 | ++ |
| 1.3 | ++ |
| 1.4 | ++ |
| 1.5 | ++ |
| 1.6 | ++ |
| 1.7 | ++ |
| 1.8 | ++ |
| 1.9 | +++ |
| 1.10 | ++ |
| 1.11 | +++ |
| 1.12 | +++ |
| 1.13 | +++ |
| 1.14 | +++ |
| 1.15 | +++ |
| 1.16 | +++ |
| 1.19 | ++ |
| 1.20 | ++ |
| 1.21 | ++ |
| 1.22 | ++ |
| 1.23 | ++ |
| 1.24 | ++ |
| 1.25 | ++ |
| 1.26 | +++ |
| 1.27 | +++ |
| 1.30 | +++ |
| 1.31 | ++ |
| 1.32 | ++ |
| 1.33 | ++ |
| 1.34 | ++ |
| 1.35 | ++ |
| 1.38 | + |
| 1.39 | + |
| 1.40 | ++ |
| 1.41 | ++ |
| 1.42 | +++ |
| 1.43 | +++ |
| 1.44 | +++ |
| 1.45 | +++ |
| 1.46 | +++ |
| 1.48 | ++ |
| 1.49 | ++ |
| 1.50 | ++ |
| 1.51 | ++ |
| 1.52 | ++ |
| 1.53 | + |
| 1.55 | + |
| 1.56 | +++ |
| 1.57 | +++ |
| 1.58 | ++ |
| 1.59 | +++ |
| 1.60 | +++ |
| 1.61 | +++ |

Table 13 Key: Compounds having an IC₅₀ value between 0.1 nM and 10 nM are denoted as ++++, 10 nM and 100 nM as +++, 100 nM and 1000 nM as ++, and 1000 nM to 10,000 nM as +.

### Example 17

### Epitope Binning of Anti-ASGR1 Antibodies to Human ASGR1 Extracellular Domain

This example shows that antibodies of this disclosure raised against human ASGR1 extracellular domain (ECD) bind to several different epitopes as tested by simultaneous binding with control anti-ASGR1 antibodies ASGR1 mAb-A, ASGR1 mAb-B, and ASGR1 mAb-C. Chimeric antibodies were chG2D (heavy chain SEQ ID NO:150; light chain SEQ ID NO:206), chH8K (heavy chain SEQ ID NO:153; light chain SEQ ID NO:209), chJ4F (heavy chain SEQ ID NO:154; light chain SEQ ID NO:210), chK2E (heavy chain SEQ ID NO:151; light chain SEQ ID NO:207), chL4L (heavy chain SEQ ID NO:152; light chain SEQ ID NO:208), and their corresponding parental antibodies were G2D, H8K, J4F, K2E, and L4L, were analyzed.

Antibody epitope binning experiment utilizing OCTET® (ForteBio, Inc.) kinetic analysis (observing competitive versus simultaneous binding to human ASGR1 ECD) revealed a competitive blocking profile of four (4) different epitope bins, referred to as bins A, B, C, and D (see Table 14). Briefly, human Fc-fused ASGR1 ECD was immobilized on anti-human IgG Fc Capture (AHC) biosensors and incubated with mouse IgG2a isotype control antibodies ASGR1 mAb-A, ASGR1 mAb-B, and ASGR1 mAb-C until saturation was reached in kinetics buffer (PBS+1% BSA+0.2% Tween20+0.9 mM CaCl₂) at pH 7.4), followed by binding of anti-ASGR1 antibodies G2D, H8K, J4F, K2E, and L4L of this disclosure. Recombinant chG2D, chH8K, chJ4F, chK2E, and chL4L antibodies were tested using mouse Fc-fused ASGR1 ECD immobilized on anti-mouse IgG Fc Capture (AMC) biosensors and incubated with control human IgG1 isotype antibodies ASGR1 mAb-A, ASGR1 mAb-C, and ASGR1 mAb-C. The interactions of the anti-ASGR1 antibodies of this disclosure and control mAbs with human ASGR1 ECD were analyzed using the OCTET® Data Analysis Software 9.0 (ForteBio).

Unexpectedly, anti-ASGR1 antibodies H8K and L4L of this disclosure were observed to bind in the presence of all 3 control mAbs and, therefore, were determined to belong to a unique epitope bin. Antibodies H8K and L4L belong to the same bin because they were found to block binding of each other, denoted bin D in Table 14 below. The other three antibodies of this disclosure (G2D, J4F, and K2E) binned with control antibody ASGR1 mAb-B.

TABLE 14
Epitope Bins of Anti-ASGR1 Antibodies to Human ASGR1 ECD*

| Antibody | mAb-A | mAb-B | mAb-C | L4L | Bin |
|---|---|---|---|---|---|
| ASGR1 mAb-A | − | + | + | + | A |
| ASGR1 mAb-B | + | − | + | + | B |
| G2D | + | − | + | + | B |
| K2E | + | − | + | + | B |
| J4F | + | − | + | + | B |
| ASGR1 mAb-C | + | + | − | + | C |
| H8K | + | + | + | − | D |
| L4L | + | + | + | − | D |

*If binding of antibody was blocked by control antibody, test antibody is assigned (−). If test antibody bound in presence of control antibody, test antibody is assigned (+).

Additional epitope binning was performed using chimeric antibodies chG2D, chJ4F, and chL4L. The procedure outlined above was essentially repeated with the following exceptions: OCTET® biosensor was AMC, antigen reagent used was mouse Fc-fused human ASGR1 and control antibodies contained human IgG1 backbone. Data was in consistent with the data in Table 14 above.

### Example 18

### Binding of Anti-ASGR1 Antibodies to an ASGR1-Expressing Tumor Cell Line

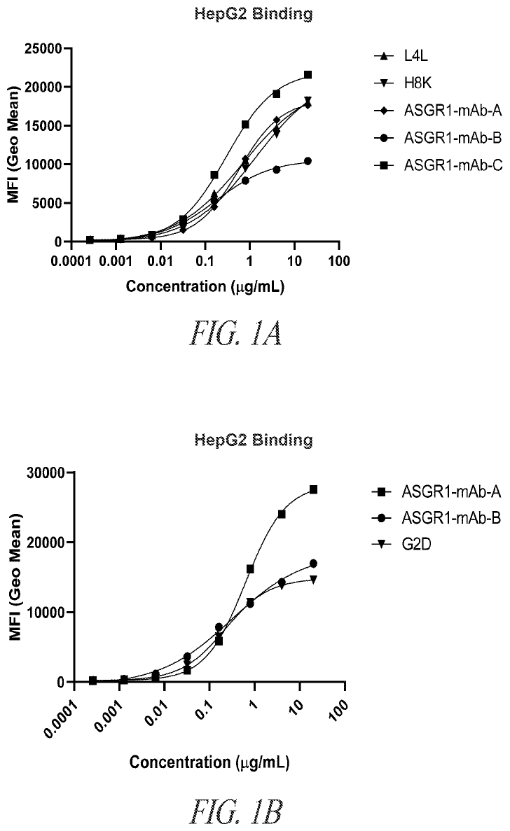

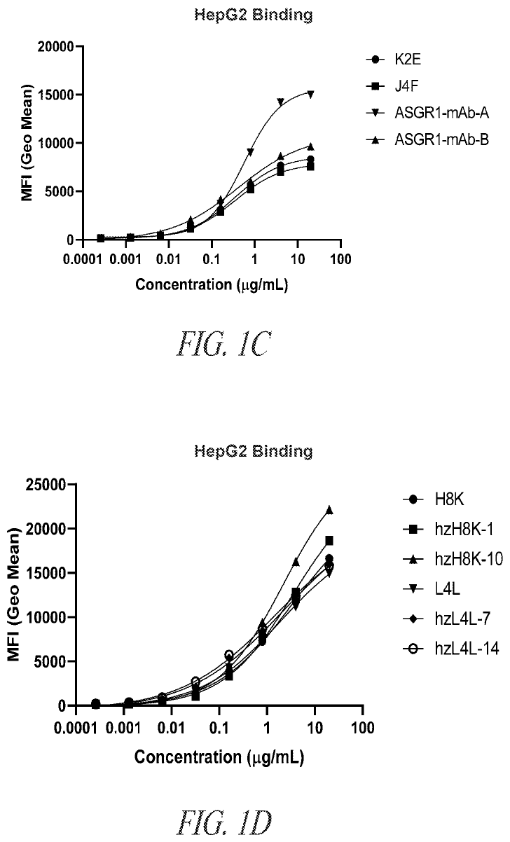

FIGS. 1A-1D show binding of various anti-ASGR1 antibodies to ASGR1-expressing cell line HepG2. Briefly, 5×10⁴ HepG2 cells/well were contacted with titrating concentrations of anti-ASGR1 antibodies in FACS Buffer (FB: PBS+2% FBS+1 mM CaCl₂)) for 30 minutes at 4° C. Cells were washed twice in FB, then incubated in anti-human IgG PE secondary for 30 minutes at 4° C. Cells were washed twice in FB, then analyzed by flow cytometry. FIGS. 1A-1C show the binding of mouse anti-ASGR1 antibodies L4L, H8K, J4F, K2E, or G2D and examined for binding as compared to control antibodies ASGR1-mAb-A, ASGR1-mAb-B, and ASGR1-mAb-C (FIG. 1A), or ASGR1-mAb-A and ASGR1-mAb-B (FIGS. 1B and 1C). Each of the test mouse anti-ASGR1 antibodies bound at a level similar to the control anti-ASGR1 antibodies. FIG. 1D shows binding of humanized anti-ASGR1 antibodies hzL4L-7 and hzL4L-14 as compared to parent mouse anti-ASGR1 antibody L4L, and binding of humanized anti-ASGR1 antibodies hzH8K-1 and hzH8K-10 as compared to parent mouse anti-ASGR1 antibody H8K. Each of the humanized anti-ASGR1 antibodies bound to the ASGR1-expressing cell line HepG2 as well as or better than the parent mouse antibodies. No binding was detected in ASGR1 negative cell line HEK293 (data not shown).

### Example 19

### Cross-Reactivity of Anti-Human ASGR1 Mabs to Cynomolgus macaque and Rat ASGR1

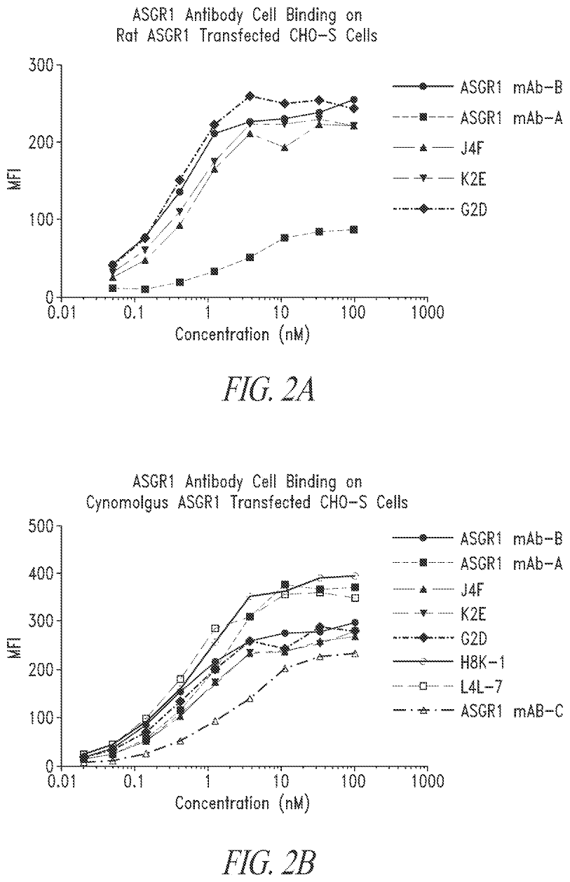

Anti-human ASGR1 antibodies of this disclosure were examined for cross-reactivity with Cynomolgus macaque and rat ASGR1. Briefly, sequence encoding Cynomolgus macaque or rat ASGR1 was transiently transfected into CHO-S cells. Transfected cells were before performing cell binding assay. A total of about 1.5×10⁴ transfected cells/well were added to 96-well V-bottom assay plate, incubated at 37° C., 5% CO2 for 48-72 hours, and then washed with culture medium before adding 100 μl of each ASGR1 antibody at different concentrations starting from 100 nM and 3-fold dilutions to a total of eight wells. The cells with antibody were incubated for 40 minutes at 4° C., then washed three times with 200 μl of fresh culture medium. To each well was added 50 μl PE conjugated goat anti-human Fc (SouthernBiotech) at 1:200 dilution in culture medium and then incubated at 4° C. for 20 minutes. After washing the plate once, the treated cells were resuspended in 200 μl culture medium and then the stained cells were analyzed on GUAVA® using Incyte software. Table 15 (see also FIG. 2B) shows that all of the anti-ASGR1 antibodies of this disclosure cross-react with Cynomolgus macaque ASGR1, whereas Table 16 (see also FIG. 2A) shows that only antibodies G2D, K2E, and J4F were capable of binding rat ASGR1 (mAbs H8K-1 and L4L-7 were not rat cross-reactive (data not shown)).

TABLE 15
Binding Cell Surface Expressed Cynomologous ASGR1

| Sample ID | EC₅₀ (nM) |
|---|---|
| ASGR1 mAb-A | 1.07 |
| ASGR1 mAb-B | 0.36 |
| G2D | 0.47 |
| K2E | 0.68 |
| J4F | 0.67 |
| ASGR1 mAb-C | 2.35 |
| H8K-1 | 0.66 |
| L4L-7 | 0.39 |

TABLE 16
Binding Cell Surface Expressed Rat ASGR1

| Anti-ASGR1 mAb | EC₅₀ (nM) |
|---|---|
| Control mAb-A | 3.22 |
| Control mAb-B | 0.38 |
| G2D | 0.37 |
| K2E | 0.52 |
| J4F | 0.62 |

### Example 20

### Cross-Reactivity of Anti-ASGR1 Antibodies to Rat CLEC10A

Anti-human ASGR1 antibodies of this disclosure were examined for cross-reactivity with Cynomolgus macaque and rat ASGR1. Briefly, sequence encoding Cynomolgus macaque or rat ASGR1 was transiently transfected into CHO-S cells. Transfected cells were before performing cell binding assay. A total of about 1.5×10⁴ transfected cells/well were added to 96-well V-bottom assay plate, incubated at 37° C., 5% CO2 for 48-72 hours, and then washed with culture medium before adding 100 μl of each ASGR1 antibody at different concentrations starting from 100 nM and 3-fold dilutions to a total of eight wells. The cells with antibody were incubated for 40 minutes at 4° C., then washed three times with 200 μl of fresh culture medium. To each well was added 50 μl PE conjugated goat anti-human Fc (SouthemBiotech) at 1:200 dilution in culture medium and then incubated at 4° C. for 20 minutes. After washing the plate once, the treated cells were resuspended in 200 μl culture medium and then the stained cells were analyzed on GUAVA® using Incyte software. Table 17 (see also FIG. 2B) shows that all of the anti-ASGR1 antibodies of this disclosure cross-react with Cynomolgus macaque ASGR1, whereas Table 18 (see also FIG. 2A) shows that only antibodies G2D, K2E, and J4F were capable of binding rat ASGR1 (mAbs H8K-1 and L4L-7 were not rat cross-reactive (data not shown)).

TABLE 17
Binding Cell Surface Expressed Cynomologous ASGR1

| Sample ID | EC₅₀ (nM) |
|---|---|
| ASGR1 mAb-A | 1.07 |
| ASGR1 mAb-B | 0.36 |
| G2D | 0.47 |
| K2E | 0.68 |
| J4F | 0.67 |
| ASGR1 mAb-C | 2.35 |
| H8K-1 | 0.66 |
| L4L-7 | 0.39 |

TABLE 18
Binding Cell Surface Expressed Rat ASGR1

| Anti-ASGR1 mAb | EC₅₀ (nM) |
|---|---|
| Control mAb-A | 3.22 |
| Control mAb-B | 0.38 |
| G2D | 0.37 |
| K2E | 0.52 |
| J4F | 0.62 |

### Example 21

### Certain Anti-Human ASGR1 Antibodies Compete with ASGR1 Ligand (GalNac) Binding

The F(ab′)2 fragments of ASGR1 antibodies were tested for GalNac ligand blocking using competition cell binding with human ASGR1 full length DNA transiently transfected CHO-S cells. GalNac ligand was linked to a human IgG1 Fc tail (referred to herein as GalNac-hFc). Competitive cell binding method was carried out as follows: (1) transfer 200 μl human ASGR1 expressing CHO-S cells (100 k/well) into a V-bottom 96-well assay plate and the cells were washed once, harvested, and resuspended in cell culture medium; (2) resuspended cells were incubated with 100 GalNac-hFc starting with 900 nM, diluted 1:2 for a total of 8 concentrations in medium containing 200 nM F(ab′)2 fragments of ASGR1 antibodies, and incubated for 40 minutes at 4° C.; (3) the treated cells were washed three times, resuspended with 50 of medium containing commercial PE conjugated anti-human Fc (SouthernBiotech) as secondary antibody (1:200 dilution), and incubates for 20 minutes at 4° C.; and (4) finally the plates were washed twice and the samples were analyzed on a GUAVA® EASYCYTE™ 8HT flow cytometer. The percent of GalNac-hFc (at a 33 nM concentration) blocked from binding ASGR1 due to the presence of anti-ASGR1 antibody was measured.

Table 19 (below) shows that even though antibodies G2D and K2E epitope binned with control ASGR1 mAb-B (having CDRs from anti-ASGR1 antibody 72G9), they differ from ASGR1 mAb-B because G2D does not compete with ASGR1 ligand (GalNac) binding and K2E only minimally blocks GalNac binding. In this regard, G2D and K2E are more like control ASGR1 mAb-C (having CDRs from anti-ASGR1 antibody 4F3). In contrast, the antibodies showing a unique epitope bin, H8K and L4L, strongly block GalNac binding to ASGR1.

TABLE 19
Anti-ASGR1 mAb Competition with
ASGR1 Ligand (GalNac) Binding

| Antibody | % Blocking GalNac Binding |
|---|---|
| ASGR1 mAb-A | 97.6 |
| ASGR1 mAb-B | 42.8 |
| G2D | −0.3 |
| K2E | 10.0 |
| J4F | −32.1 |
| ASGR1 mAb-C | −5.4 |
| hzH8K-1 | 81.3 |
| hzL4L-14 | 90.7 |

### Example 22

### Effect of Calcium on Anti-ASGR1 Binding to ASGR1

The ability of anti-human ASGR1 antibodies of this disclosure to bind to human ASGR1 in the presence or absence of calcium in binding buffer was examined. Briefly, ASGR1 antibodies were bound to immobilized human ASGR1 extracellular domain (ECD) at saturating levels and subsequently dissociated under three different conditions as follows: (a) binding and dissociation with buffer containing calcium (PBS+1% BSA+0.2% Tween20+0.9 mM CaCl₂), pH 7.4); (b) binding with buffer containing calcium and dissociation with buffer lacking calcium; and (c) binding and dissociation with buffer lacking calcium. Analysis of ASGR1 antibody binding to human ASGR1 ECD was performed using the OCTET® Red 96 instrument and analyzed using the OCTET®Data Analysis Software 9.0 (ForteBio). His-tagged human ASGR1 ECD was immobilized to OCTET® Penta-His biosensors and ASGR1 antibodies were subjected to avidity binding under the conditions described above. Association with the ASGR1 ECD was measured by the rate of and increase in magnitude of signal response at a given antibody concentration. Dissociation from the ASGR1 ECD was measured the rate of loss in signal upon removal of the antibody and further incubation with or without Ca²⁺. Tested in these assays were chimeric anti-ASGR1 antibodies chG2D, chJ4F, chK2E, and chL4L of this disclosure (each comprising the variable regions of anti-ASGR1 antibodies G2D, J4F, K2E, and L4L, respectively, of this disclosure fused to human IgG1 Fc domain). Also tested were humanized antibodies hzH8K-1 and hzL4L-7. Control antibodies were ASGR1 mAb-A, ASGR1 mAb-B, and ASGR1 mAb-C.

Tested mAbs were considered sensitive to calcium when saturation binding of ASGR1 mAb to human ASGR1 ECD in the presence of calcium showed a difference in binding signal after dissociation in the presence of calcium that was >±10% as compared to the binding signal after dissociation in the absence of calcium (i.e., calcium sensitivity refers to antibodies that appear to require Ca²⁺ to remain bound to ASGR1 ECD). Antibodies lacking sensitivity to calcium included chG2D, chH8K, chK2E, hzL4L-7, and control antibodies ASGR1 mAb-A and ASGR1 mAb-B. In contrast, chJ4F showed calcium sensitivity since this antibody exhibited a binding signal decrease of 67% from saturation when dissociation was carried out in the absence of calcium, whereas only 10% of binding signal loss was observed when the dissociation step was carried out in the presence of calcium. Control antibody ASGR1 mAb-C shows a dramatic loss in binding signal when dissociation is carried out in the presence (73% loss) or absence (93% loss) of calcium during the dissociation step. The binding dissociation by control antibody ASGR1 mAb-C appears to be affected by calcium, but its binding profile has a unique response to calcium since its association to ligand is not affected by calcium.

Further analysis of the antibodies of this disclosure was to determine whether the presence of calcium was needed for binding (i.e., during association) of the antibodies to human ASGR1 ECD. Under these conditions, several antibodies displayed the complete inability to bind to human ASGR1 ECD (i.e., calcium dependent binding). Table 20 provides a summary of the calcium sensitivity results.

TABLE 20
Calcium Sensitivity of ASGR1 Antibodies

| | Ca²⁺ Sensitivity during: | | Ca²⁺ |
|---|---|---|---|
| mAb | Association | Dissociation | Sensitivity |
| ASGR1 mAb-A | N | N | N |
| ASGR1 mAb-B | Y | N | Y |
| chG2D | Y | N | Y |
| chK2E | Y | N | Y |
| chJ4F | Y | Y | Y |
| ASGR1 mAb-C | N | Y | N |
| hzH8K-1 | N | N | N |
| hzL4L-7 | N | N | N |

N = No;
Y = Yes

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet (including U.S. application 62/912,856, filed Oct. 9, 2019 and 63/047,150, filed Jul. 1, 2020) are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A conjugate, comprising:
(a) an antibody construct comprising an antigen binding domain that specifically binds to a first antigen on a liver cell, wherein the first antigen is ASGR1;
(b) a TGFβR1 inhibitor is a compound of Formula (A-I), (A-IA), (A-IB), (A-IC), (A-ID), (A-IE), (B-I), (B-Ia), (B-Ib), (B-Ic), (B-Id), or (B-Ie); and
(c) a linker covalently attached to the TGFβR1 inhibitor and to the antibody construct;
wherein the antibody construct or the antigen binding domain specific for ASGR1 comprises a heavy chain variable region (VH) and a light chain variable region (VL), wherein:
(i) the VH comprises a CDR1 (VH-CDR1) comprising the amino acid sequence of SEQ ID NO:1, a VH-CDR2 comprising the amino acid sequence selected from SEQ ID NO:8, a VH-CDR3 comprising the amino acid sequence of SEQ ID NO:13; and the VL comprises a CDR1 (VL-CDR1) comprising the amino acid sequence of SEQ ID NO:18, a VL-CDR2 comprising the amino acid sequence selected from any one of SEQ ID NO:23, and a VL-CDR3 comprising the amino acid sequence of SEQ ID NO:33;
(ii) the VH comprises a CDR1 (VH-CDR1) comprising the amino acid sequence of SEQ ID NO:1, a VH-CDR2 comprising the amino acid sequence selected from SEQ ID NO:6, a VH-CDR3 comprising the amino acid sequence of SEQ ID NO:13; and the VL comprises a CDR1 (VL-CDR1) comprising the amino acid sequence of SEQ ID NO:18, a VL-CDR2 comprising the amino acid sequence selected from any one of SEQ ID NO:23, and a VL-CDR3 comprising the amino acid sequence of SEQ ID NO:33;
(iii) the VH comprises a CDR1 (VH-CDR1) comprising the amino acid sequence of SEQ ID NO:2, a VH-CDR2 comprising the amino acid sequence selected from SEQ ID NO:9, a VH-CDR3 comprising the amino acid sequence of SEQ ID NO:14; and the VL comprises a CDR1 (VL-CDR1) comprising the amino acid sequence of SEQ ID NO:19, a VL-CDR2 comprising the amino acid sequence selected from any one of SEQ ID NO:26, and a VL-CDR3 comprising the amino acid sequence of SEQ ID NO:34;
(iv) the VH comprises a CDR1 (VH-CDR1) comprising the amino acid sequence of SEQ ID NO:1, a VH-CDR2 comprising the amino acid sequence selected from any one of SEQ ID NOS:6-8, a VH-CDR3 comprising the amino acid sequence of SEQ ID NO:13; and the VL comprises a CDR1 (VL-CDR1) comprising the amino acid sequence of SEQ ID NO:18, a VL-CDR2 comprising the amino acid sequence selected from any one of SEQ ID NOS:23-25, and a VL-CDR3 comprising the amino acid sequence of SEQ ID NO:33;
(v) the VH comprises a CDR1 (VH-CDR1) comprising the amino acid sequence of SEQ ID NO:2, a VH-CDR2 comprising the amino acid sequence of SEQ ID NO:9, a VH-CDR3 comprising the amino acid sequence of SEQ ID NO:14; and the VL comprises a CDR1 (VL-CDR1) comprising the amino acid sequence of SEQ ID NO:19, a VL-CDR2 comprising the amino acid sequence of SEQ ID NO:26 or SEQ ID NO:27, and a VL-CDR3 comprising the amino acid sequence of SEQ ID NO:34;
(vi) the VH comprises a CDR1 (VH-CDR1) comprising the amino acid sequence of SEQ ID NO:5, a VH-CDR2 comprising the amino acid sequence of SEQ ID NO:12, a VH-CDR3 comprising the amino acid sequence of SEQ ID NO:17; and the VL comprises a CDR1 (VL-CDR1) comprising the amino acid sequence of SEQ ID NO:22, a VL-CDR2 comprising the amino acid sequence of SEQ ID NO:32, and a VL-CDR3 comprising the amino acid sequence of SEQ ID NO:37;
(vii) the VH comprises a CDR1 (VH-CDR1) comprising the amino acid sequence of SEQ ID NO:3, a VH-CDR2 comprising the amino acid sequence of SEQ ID NO:10, a VH-CDR3 comprising the amino acid sequence of SEQ ID NO:15; and the VL comprises a CDR1 (VL-CDR1) comprising the amino acid sequence of SEQ ID NO:20, a VL-CDR2 comprising the amino acid sequence selected from any one of SEQ ID NOS:28-30, and a VL-CDR3 comprising the amino acid sequence of SEQ ID NO:35; or
(viii) the VH comprises a CDR1 (VH-CDR1) comprising the amino acid sequence of SEQ ID NO:4, a VH-CDR2 comprising the amino acid sequence of SEQ ID NO:11, a VH-CDR3 comprising the amino acid sequence of SEQ ID NO:16; and the VL comprises a CDR1 (VL-CDR1) comprising the amino acid sequence of SEQ ID NO:21, a VL-CDR2 comprising the amino acid sequence of SEQ ID NO:31, and a (VL-CDR3) comprising the amino acid sequence of SEQ ID NO:3.

2. The conjugate of claim 1, wherein the antigen binding domain is humanized.

3. The conjugate of claim 1, wherein the antigen binding domain comprises:
(a) a VH comprising an amino acid sequence that has at least 90% identity with the amino acid sequence selected from any one of SEQ ID NOS:240, 239, 43-88, 238, and 241-243, and a VL comprising an amino acid sequence that has at least 90% identity with the amino acid sequence selected from any one of SEQ ID NOS:247, 131-133, 244-246, 248, and 249, provided that the amino acid sequences of the VH-CDRs and VL-CDRs are unchanged; or
(b) a VH comprising an amino acid sequence that has at least 90% identity with the amino acid sequence of SEQ ID NO:240, and a VL comprising an amino acid sequence that has at least 90% identity with the amino acid sequence of SEQ ID NO:247, provided that the amino acid sequences of the VH-CDRs and VL-CDRs are unchanged; or
(c) a VH comprising an amino acid sequence that has at least 90% identity with the amino acid sequence of SEQ ID NO:239, and a VL comprising an amino acid sequence that has at least 90% identity with the amino acid sequence of SEQ ID NO:247, provided that the amino acid sequences of the VH-CDRs and VL-CDRs are unchanged; or
(d) a VH comprising an amino acid sequence that has at least 90% identity with the amino acid sequence of SEQ ID NO:240, and a VL comprising an amino acid sequence that has at least 90% identity with the amino acid sequence of SEQ ID NO:248, provided that the amino acid sequences of the VH-CDRs and VL-CDRs are unchanged; or
(e) a VH comprising an amino acid sequence that has at least 90% identity with the amino acid sequence of SEQ ID NO:239, and a VL comprising an amino acid sequence that has at least 90% identity with the amino acid sequence of SEQ ID NO:248, provided that the amino acid sequences of the VH-CDRs and VL-CDRs are unchanged; or
(f) a VH comprising the amino acid sequence selected from any one of SEQ ID NOS:240, 239, 43-88, 238, and 241-243, and a VL comprising the amino acid sequence selected from any one of SEQ ID NOS:247, 131-133, 244-246, 248, and 249; or
(g) a VH comprising the amino acid sequence of SEQ ID NO:240, and a VL comprising the amino acid sequence of SEQ ID NO:247; or
(h) a VH comprising the amino acid sequence of SEQ ID NO:239, and a VL comprising the amino acid sequence of SEQ ID NOS:247; or
(i) a VH comprising the amino acid sequence of SEQ ID NO:240, and a VL comprising the amino acid sequence of SEQ ID NO:248; or
(j) a VH comprising the amino acid sequence of SEQ ID NO:239, and a VL comprising the amino acid sequence of SEQ ID NO:248.

4.-7. (canceled)

8. The conjugate of claim 1, wherein the antibody construct comprises:
(a) a heavy chain comprising an amino acid sequence that is at least 90% identity with the amino acid sequence selected from any one of SEQ ID NOS:155-200, and a light chain comprising an amino acid sequence that has at least 90% identity with the amino acid sequence selected from any one of SEQ ID NOS:211-213, provided that the amino acid sequences of the VH-CDRs and VL-CDRs are unchanged; or
(b) a heavy chain comprising an amino acid sequence selected from any one of SEQ ID NOS:155-200, and a light chain comprising the amino acid sequence selected from any one of SEQ ID NOS:211-213.

9. (canceled)

10. The conjugate of claim 1, wherein the antibody construct comprises an Fc domain covalently attached to the antigen binding domain.

11. The conjugate of claim 10, wherein the Fc domain is an IgG Fc domain.

12. The conjugate of claim 11, wherein the Fc domain is an IgG1 Fc domain.

13. The conjugate of claim 10, wherein the Fc domain is an Fc domain variant comprising one or more amino acid substitutions in an IgG Fc domain as compared to an amino acid sequence of a wild-type IgG Fc domain.

14. The conjugate of claim 13, wherein the Fc domain variant has increased affinity to one or more Fcγ receptors as compared to the wild-type IgG Fc domain.

15. The conjugate of claim 10, wherein the Fc domain is covalently attached to the antigen binding domain:
(a) as an Fc domain-antigen binding domain fusion protein; or
(b) by conjugation via a second linker.

16. The conjugate of claim 10, wherein the antibody construct has a K_{d} for binding of the Fc domain to an Fc receptor in the presence of the TGFβR1 inhibitor and wherein the K_{d} for binding of the Fc domain to the Fc receptor in the presence of the TGFβR1 inhibitor is no greater than about 100 times a K_{d} for binding of the Fc domain to the Fc receptor in the absence of the TGFβR1 inhibitor.

17. The conjugate of claim 1, wherein the conjugate is represented by Formula (I): 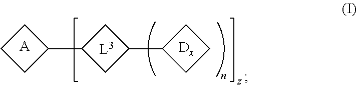
wherein:
A is the ASGR1 or ASGR2 antibody construct,
L³ is the linker;
Dₓ is the TGFβR1 inhibitor;
n is selected from 1 to 20; and
z is selected from 1 to 20.

18. The conjugate of claim 17, wherein z is from 2 to 20, or 2 to 10, or 2 to 8, or 1 to 8.

19. The conjugate of claim 17, wherein n is 1 and z is from 1 to 8.

20. The conjugate of claim 1, wherein the antibody construct comprises an antibody.

21.-30. (canceled)

31. The conjugate of claim 1, wherein the TGFβR1 inhibitor is a compound of Formula (A-I): 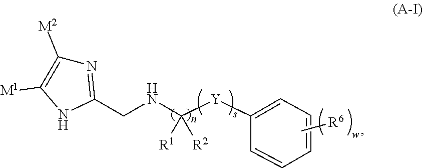
wherein
one of M¹ and M² is 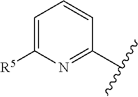
and the other of M¹ and M² is selected from: 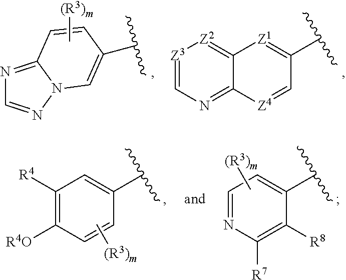
R¹ and R² are, at each occurrence, independently selected from hydrogen, halogen, —OR¹¹, —SR¹¹, —N(R¹¹)₂, —NO₂, —CN, phenyl, and —C₁-C₆ alkyl, wherein said —C₁-C₆ alkyl is optionally substituted with one or more substituents independently selected from halogen, —OR¹¹, —SR¹¹, —S(O)R¹⁰, —S(O)₂R¹¹, —S(O)₂N(R¹¹)₂—N(R¹¹)₂, —C(O)R¹⁰, —C(O)N(R¹¹)₂, —N(R¹¹)C(O)R¹⁰, —C(O)OR¹¹, —OC(O)R¹⁰, —NO₂, and —CN;
R³ is, at each occurrence, independently selected from halogen, —C₁-C₃ alkyl, —C₁-C₃ haloalkyl, —OH, —NO₂, —CN, —OC₁-C₃ alkyl, and —OC₁-C₃ haloalkyl;
each R⁴ is, at each occurrence, independently selected from hydrogen and C₁-C₃ alkyl or two R⁴ join together with atoms to which they are attached to form a 5- or 6-membered heterocycle optionally substituted with one or more substituents independently selected from halogen, C₁-C₃ alkyl, —OH, OC₁-C₃ alkyl, and —OC₁-C₃ haloalkyl;
R⁵ is hydrogen, halogen, —OR⁶¹, —SR⁶¹, —N(R⁶¹)₂, —NO₂, —CN, and —C₁-C₆ alkyl, wherein said —C₁-C₆ alkyl is optionally substituted with one or more substituents independently selected from halogen, —OR⁶¹, —SR⁶¹, —N(R⁶¹)₂, —NO₂, and —CN;
R⁶ is, at each occurrence, independently selected from:
halogen, —OR²¹, —SR²¹, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)₂, —NO₂, and —CN;
C₁-C₁₀ alkyl, C₂-C₁₀ alkenyl, and C₂-C₁₀ alkynyl, each of which is optionally substituted with one or more substituents independently selected from halogen, —OR²¹, —SR²¹, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)₂, —NO₂, ═O, ═S, ═N(R²¹), —CN, a C₃-C₁₀ carbocycle, and a 3- to 10-membered heterocycle wherein said C₃-C₁₀ carbocycle and said 3- to 10-membered heterocycle are optionally substituted with one or more substituents independently selected from R^{X}; and
a C₃-C₁₀ carbocycle and a 3- to 10-membered heterocycle, each of which is optionally substituted with one or more substituents independently selected from halogen, —OR²⁰, —OH, —SR²⁰, —SH, —N(R²¹)₂, —C(O)R²⁰, —C(O)N(R²¹)₂, —N(R²¹)C(O)R²⁰, —C(O)OR²¹, —OC(O)R²¹, —S(O)R²⁰, —S(O)₂R²¹, —S(O)₂N(R²¹)₂, —OC(O)OR²¹, —OC(O)N(R²¹)₂, —NR²¹C(═O)OR²¹, —N(R²¹)C(O)N(R²¹)₂, —NO₂, ═O, ═S, ═N(R²¹), —CN, —C₂-C₆ alkenyl, —C₂-C₆ alkynyl and C₁-C₆ alkyl wherein said C₁-C₆ alkyl is optionally substituted with one or more substituents independently selected from R^{Y};
R⁷ and R⁸ are independently selected from hydrogen, halogen, C₁-C₃ alkyl, —OH, OC₁-C₃ alkyl, and —OC₁-C₃ haloalkyl, or R⁷ and R⁸ join together with the atoms to which they are attached to form a C₅-C₆ carbocycle or 5- or 6-membered heterocycle each of which is optionally substituted with one or more substituents independently selected from halogen, —OR¹¹, —SR³¹, —N(R³¹)₂, —NO₂, —CN and —C₁-C₆ alkyl wherein said C₁-C₆ alkyl is optionally substituted with one or more substituents independently selected from halogen, —OR³¹, —SR³¹, —N(R³¹)₂, —NO₂, and —CN;
Y is selected from —O— and —N(R⁹)— and R⁹ is, at each occurrence, independently selected from: hydrogen; and —C₁-C₆ alkyl optionally substituted with one or more substituents independently selected from halogen, —OR⁴¹, —SR⁴¹, —S(O)R⁴⁰, —S(O)₂R⁴¹, —S(O)₂N(R⁴¹)₂, —N(R⁴¹)₂, —C(O)R⁴⁰, —C(O)N(R⁴¹)₂, —N(R⁴¹)C(O)R⁴⁰, —C(O)OR⁴¹, —OC(O)R⁴⁰, —NO₂, and —CN;
each R¹⁰, R²⁰, and R⁴⁰ is independently selected at each occurrence from:
—C₁-C₁₀ alkyl, —C₂-C₁₀ alkenyl, and —C₂-C₁₀ alkynyl, each of which is optionally substituted with one or more substituents independently selected from R^{Y}; and
a C₃-C₁₂ carbocycle and a 3- to 12-membered heterocycle, each of which is optionally substituted with one or more substituents independently selected from R^{X};
each R¹¹, R²¹, R³¹, R⁴¹, and R⁶¹ is independently selected at each occurrence from:
hydrogen;
—C₁-C₁₀ alkyl, —C₂-C₁₀ alkenyl, and —C₂-C₁₀ alkynyl, each of which is optionally substituted with one or more substituents independently selected from R^{Y}; and
a C₃-C₁₂ carbocycle and a 3- to 12-membered heterocycle, each of which is optionally substituted with one or more substituents independently selected from R^{X},
or two R¹¹, R²¹, R³¹, R⁴¹, or R⁶¹ on the same N atom are taken together with the N atom to which they are attached to form a N-containing heterocycle optionally substituted with R^{X};
each R^{X} is independently selected at each occurrence from: halogen, —OR⁵¹, —SR⁵¹, —N(R⁵¹)₂, —C(O)R⁵⁰, —C(O)N(R⁵¹)₂, —N(R⁵¹)C(O)R⁵⁰, —C(O)OR⁵¹, —OC(O)R⁵¹, —S(O)R⁵⁰, —S(O)₂R⁵¹, —S(O)₂N(R⁵¹)₂, —OC(O)OR⁵¹, —OC(O)N(R⁵¹)₂, —NR⁵¹C(═O)OR⁵¹, —N(R⁵¹)C(O)N(R⁵¹)₂, —NO₂, ═O, ═S, ═N(R⁵¹), —CN, —C₂-C₆ alkenyl, —C₂-C₆ alkynyl, and C₁-C₆ alkyl, wherein said C₁-C₆ alkyl is optionally substituted with one or more substituents independently selected from —OR⁵¹, —SR⁵¹, —N(R⁵¹)₂, —C(O)R⁵⁰, —C(O)N(R⁵¹)₂, —N(R⁵¹)C(O)R⁵⁰, —C(O)OR⁵¹, —OC(O)R⁵¹, —S(O)R⁵⁰, —S(O)₂R⁵¹, —S(O)₂N(R⁵¹)₂, —OC(O)OR⁵¹, —OC(O)N(R⁵¹)₂, —NR⁵¹C(═O)OR⁵¹, —N(R⁵¹)C(O)N(R⁵¹)₂, and ═O;
each R^{Y} is independently selected at each occurrence from: halogen, —OR⁵¹, —SR⁵¹, —N(R⁵¹)₂, —C(O)R⁵⁰, —C(O)N(R⁵¹)₂, —N(R⁵¹)C(O)R⁵⁰, —C(O)OR⁵¹, —OC(O)R⁵¹, —S(O)R⁵⁰, —S(O)₂R⁵¹, —S(O)₂N(R⁵¹)₂, —OC(O)OR⁵¹, —OC(O)N(R⁵¹)₂, —NR⁵¹C(═O)OR⁵¹, —N(R⁵¹)C(O)N(R⁵¹)₂, —NO₂, ═O, ═S, ═N(R⁵¹), and —CN;
each R⁵⁰ is independently selected at each occurrence from:
—C₁-C₁₀ alkyl, —C₂-C₁₀ alkenyl, and —C₂-C₁₀ alkynyl, each of which is optionally substituted with one or more substituents independently selected from halogen, —OH, —CN, —NO₂, —NH₂, ═O, ═S, —O—C₁-C₁₀ alkyl, C₃-C₁₂ carbocycle, and a 3- to 12-membered heterocycle; and
a C₃-C₁₂ carbocycle and a 3- to 12-membered heterocycle, each of which is optionally substituted with one or more substituents independently selected from halogen, —OH, —CN, —NO₂, —NH₂, ═O, ═S, —C₁-C₁₀ alkyl, —O—C₁-C₁₀ alkyl, and —C₁-C₁₀ haloalkyl;
each R⁵¹ is independently selected at each occurrence from:
hydrogen;
—C₁-C₁₀ alkyl, —C₂-C₁₀ alkenyl, and —C₂-C₁₀ alkynyl, each of which is optionally substituted with one or more substituents independently selected from halogen, —OH, —CN, —NO₂, —NH₂, ═O, ═S, —O—C₁-C₁₀ alkyl, C₃-C₁₂ carbocycle, and a 3- to 12-membered heterocycle; and
a C₃-C₁₂ carbocycle and a 3- to 12-membered heterocycle, each of which is optionally substituted with one or more substituents independently selected from halogen, —OH, —CN, —NO₂, —NH₂, ═O, ═S, —C₁-C₁₀ alkyl, —O—C₁-C₁₀ alkyl, and —C₁-C₁₀ haloalkyl;
Z¹, Z², Z³, and Z⁴ are each independently selected from N or C(H);
n is selected from 1, 2, and 3;
m is 0, 1, or 2;
s is selected from 0 and 1; and
w is selected from 0, 1, 2, 3, 4, and 5;
or a salt thereof.

32. The conjugate of claim 31, wherein the compound of Formula (A-I) is a compound of Formula (A-IA), (A-IB), (A-IC), (A-ID) or (A-IE): 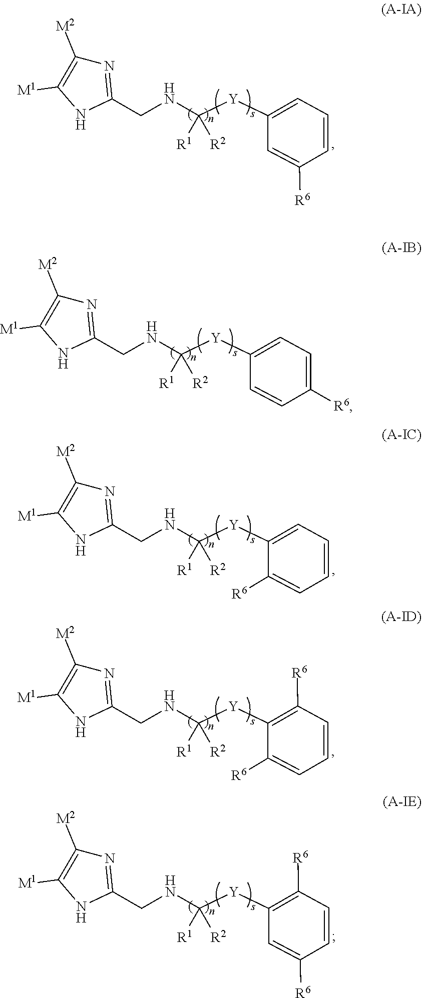
wherein the remaining variables are as defined in claim 26.

33. The conjugate of claim 31, wherein 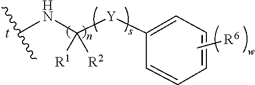
is 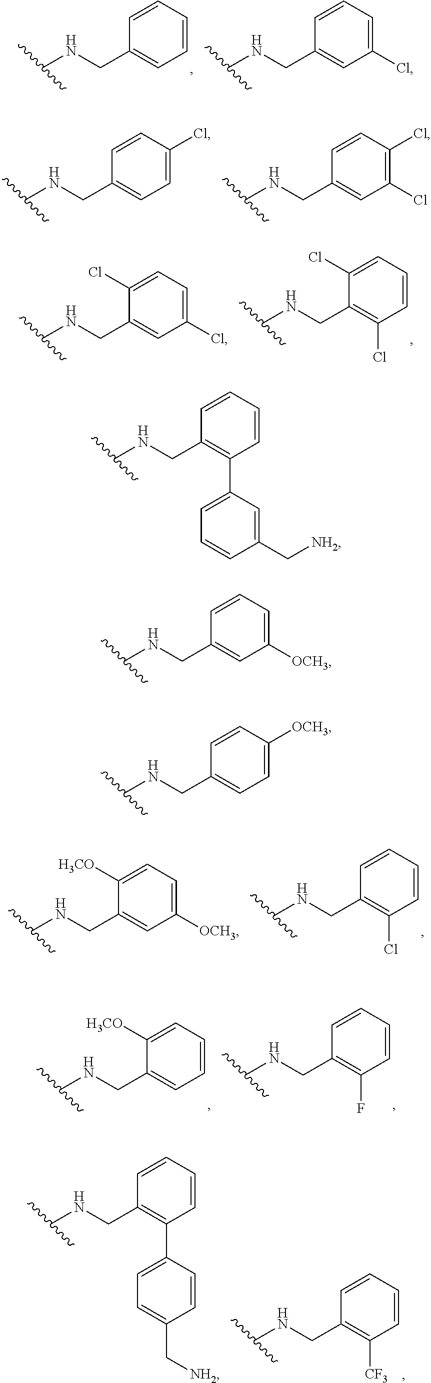 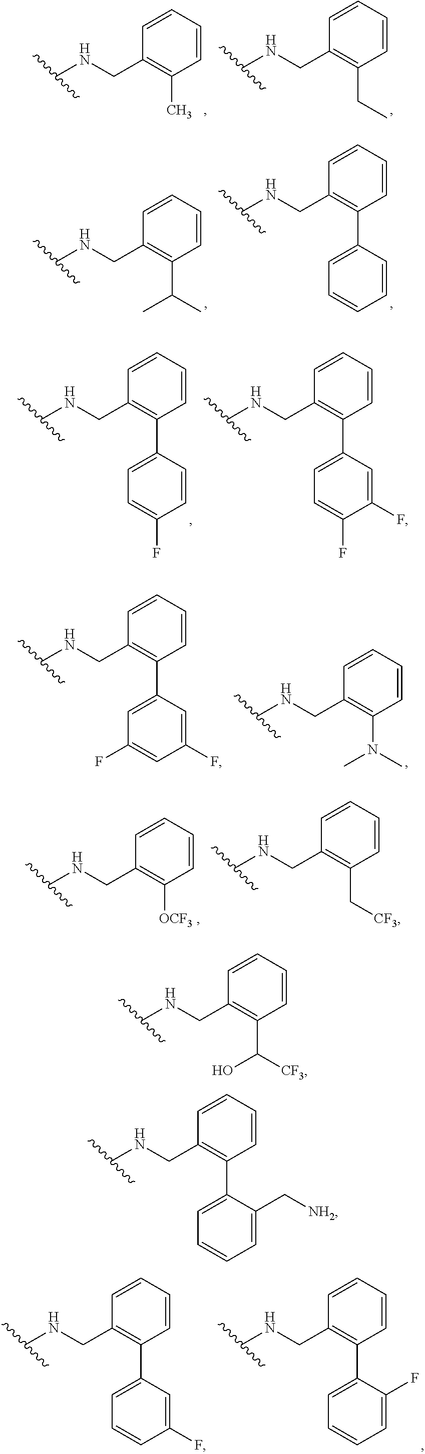 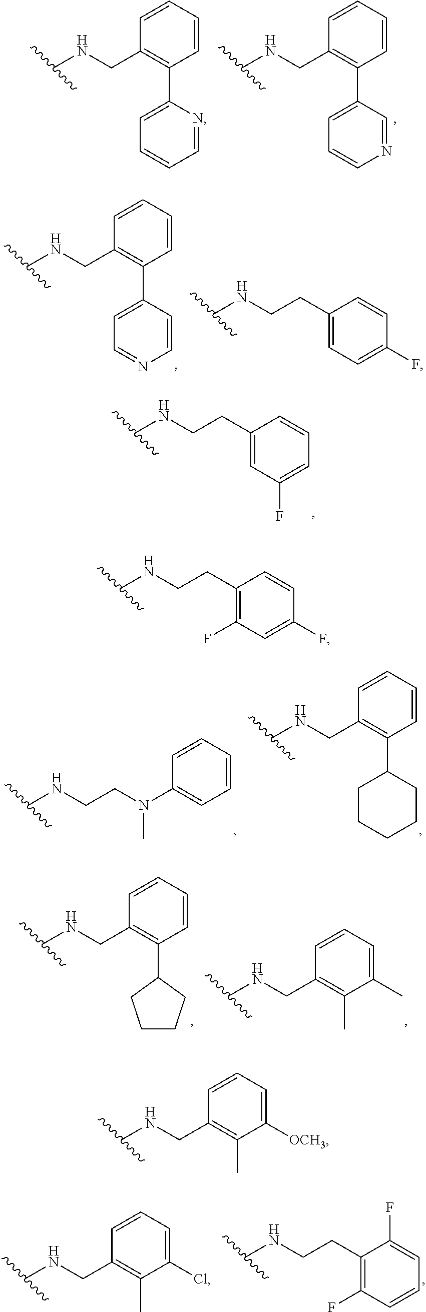 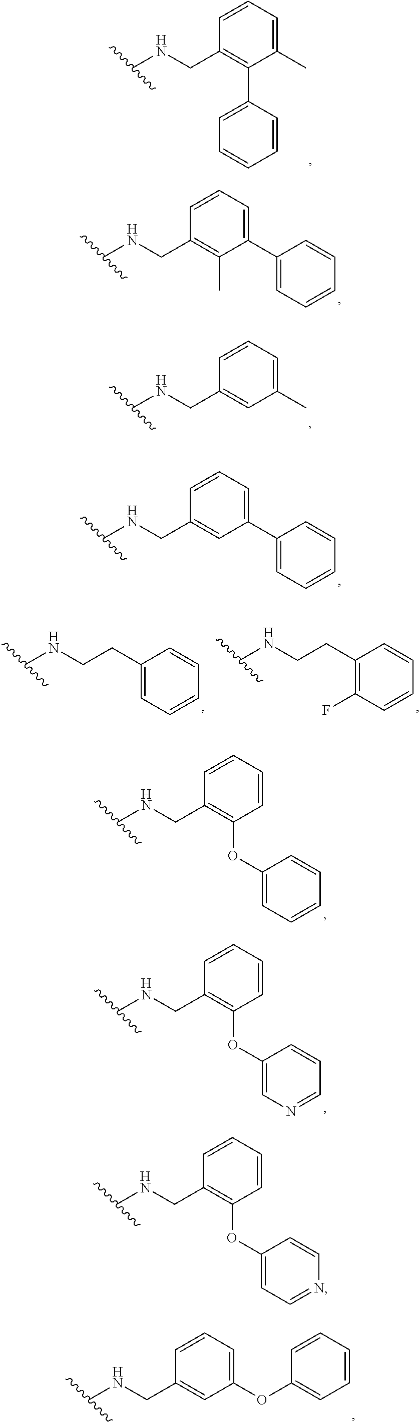 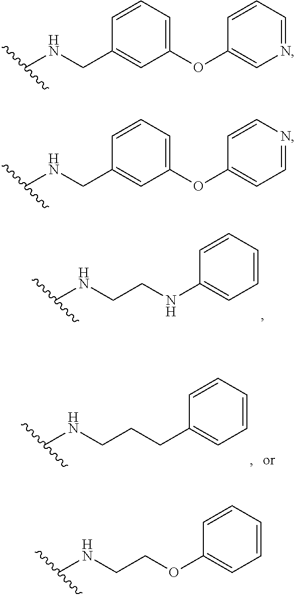
wherein [symbol] represents the point of attachment to

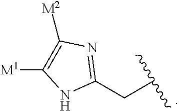

34. The conjugate of claim 1, wherein the TGFβR1 inhibitor is a compound selected from:

| Cmpd. | |
|---|---|
| No. | Structure |
| 1 | 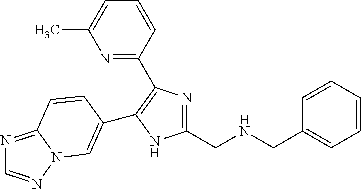 [SMILES: Cc1cccc(-c2nc(CNCc3ccccc3)[nH]c2-c2ccc3ncnn3c2)n1] |
| 2 | 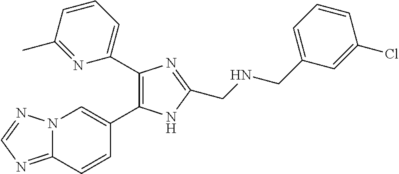 [SMILES: Cc1cccc(-c2nc(CNCc3cccc(Cl)c3)[nH]c2-c2ccc3ncnn3c2)n1] |
| 3 | 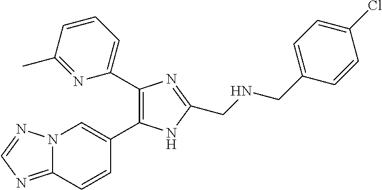 [SMILES: Cc1cccc(-c2nc(CNCc3ccc(Cl)cc3)[nH]c2-c2ccc3ncnn3c2)n1] |
| 4 | 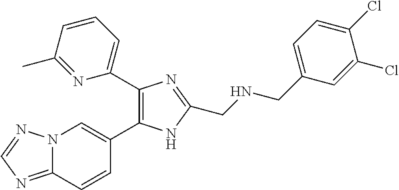 [SMILES: Cc1cccc(-c2nc(CNCc3ccc(Cl)c(Cl)c3)[nH]c2-c2ccc3ncnn3c2)n1] |
| 5 | 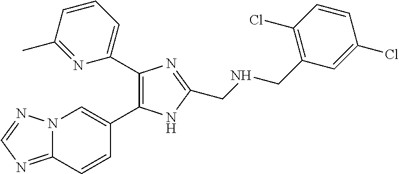 [SMILES: Cc1cccc(-c2nc(CNCc3cc(Cl)ccc3Cl)[nH]c2-c2ccc3ncnn3c2)n1] |
| 6 | 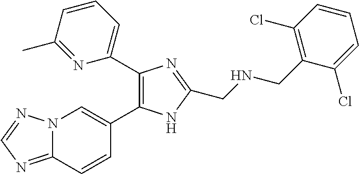 [SMILES: Cc1cccc(-c2nc(CNCc3c(Cl)cccc3Cl)[nH]c2-c2ccc3ncnn3c2)n1] |
| 7 | 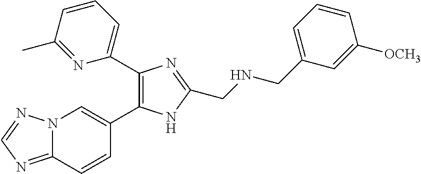 [SMILES: COc1cccc(CNCc2nc(-c3cccc(C)n3)c(-c3ccc4ncnn4c3)[nH]2)c1] |
| 8 | 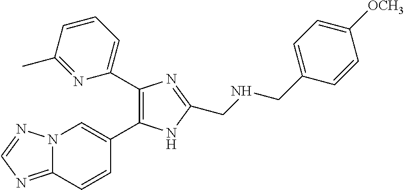 [SMILES: COc1ccc(CNCc2nc(-c3cccc(C)n3)c(-c3ccc4ncnn4c3)[nH]2)cc1] |
| 9 | 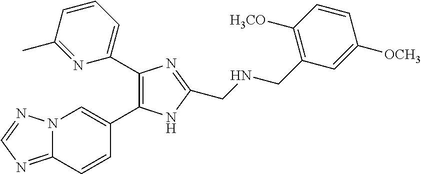 [SMILES: COc1ccc(OC)c(CNCc2nc(-c3cccc(C)n3)c(-c3ccc4ncnn4c3)[nH]2)c1] |
| 10 | 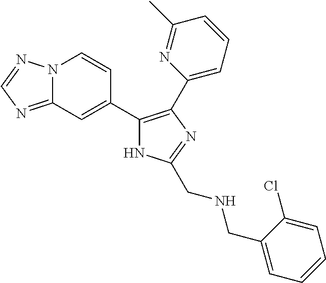 [SMILES: Cc1cccc(-c2nc(CNCc3ccccc3Cl)[nH]c2-c2ccn3ncnc3c2)n1] |
| 11 | 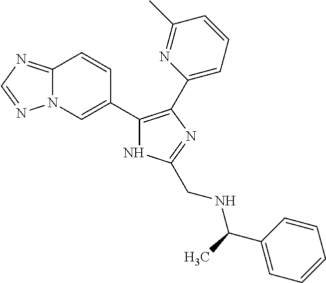 [SMILES: Cc1cccc(-c2nc(CN[C@H](C)c3ccccc3)[nH]c2-c2ccc3ncnn3c2)n1] |
| 12 | 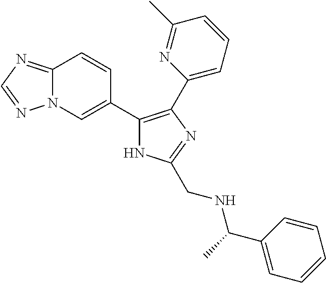 [SMILES: Cc1cccc(-c2nc(CN[C@@H](C)c3ccccc3)[nH]c2-c2ccc3ncnn3c2)n1] |
| 13 | 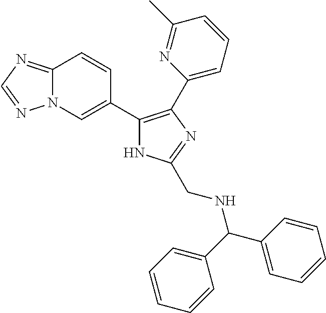 [SMILES: Cc1cccc(-c2nc(CNC(c3ccccc3)c3ccccc3)[nH]c2-c2ccc3ncnn3c2)n1] |
| 14 | 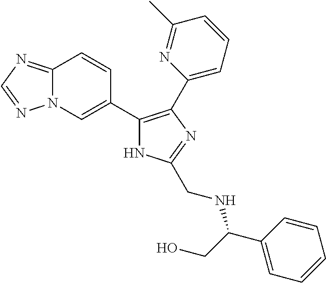 [SMILES: Cc1cccc(-c2nc(CN[C@@H](CO)c3ccccc3)[nH]c2-c2ccc3ncnn3c2)n1] |
| 15 | 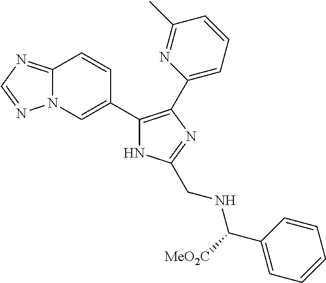 [SMILES: COC(=O)[C@H](NCc1nc(-c2cccc(C)n2)c(-c2ccc3ncnn3c2)[nH]1)c1ccccc1] |
| 16 | 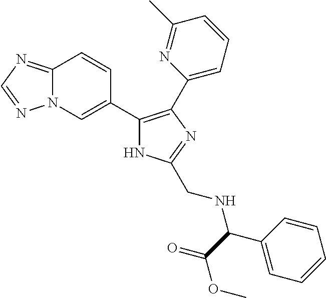 [SMILES: COC(=O)[C@@H](NCc1nc(-c2cccc(C)n2)c(-c2ccc3ncnn3c2)[nH]1)c1ccccc1] |
| 17 | 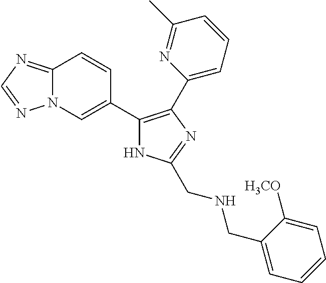 [SMILES: COc1ccccc1CNCc1nc(-c2cccc(C)n2)c(-c2ccc3ncnn3c2)[nH]1] |
| 18 | 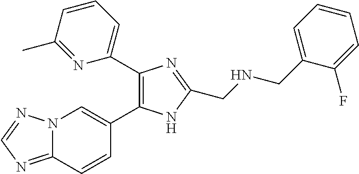 [SMILES: Cc1cccc(-c2nc(CNCc3ccccc3F)[nH]c2-c2ccc3ncnn3c2)n1] |
| 19 | 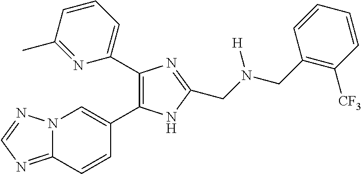 [SMILES: Cc1cccc(-c2nc(CNCc3ccccc3C(F)(F)F)[nH]c2-c2ccc3ncnn3c2)n1] |
| 20 | 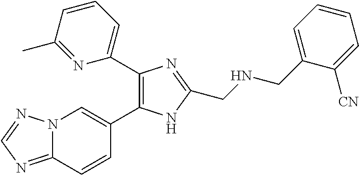 [SMILES: Cc1cccc(-c2nc(CNCc3ccccc3C#N)[nH]c2-c2ccc3ncnn3c2)n1] |
| 21 | 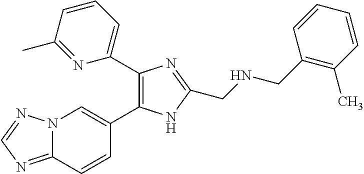 [SMILES: Cc1cccc(-c2nc(CNCc3ccccc3C)[nH]c2-c2ccc3ncnn3c2)n1] |
| 22 | 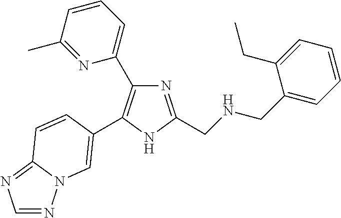 [SMILES: CCc1ccccc1CNCc1nc(-c2cccc(C)n2)c(-c2ccc3ncnn3c2)[nH]1] |
| 23 | 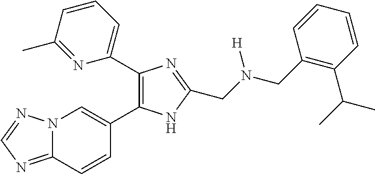 [SMILES: Cc1cccc(-c2nc(CNCc3ccccc3C(C)C)[nH]c2-c2ccc3ncnn3c2)n1] |
| 24 | 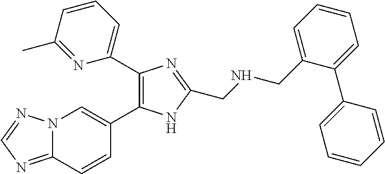 [SMILES: Cc1cccc(-c2nc(CNCc3ccccc3-c3ccccc3)[nH]c2-c2ccc3ncnn3c2)n1] |
| 25 | 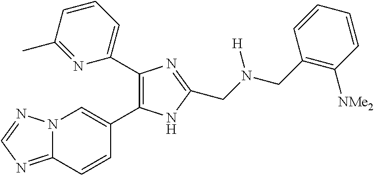 [SMILES: Cc1cccc(-c2nc(CNCc3ccccc3N(C)C)[nH]c2-c2ccc3ncnn3c2)n1] |
| 26 | 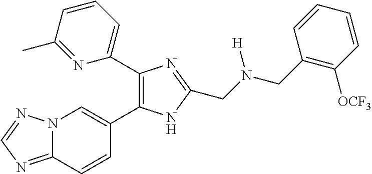 [SMILES: Cc1cccc(-c2nc(CNCc3ccccc3OC(F)(F)F)[nH]c2-c2ccc3ncnn3c2)n1] |
| 27 | 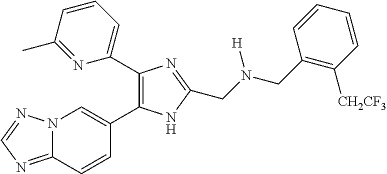 |
| 28 | 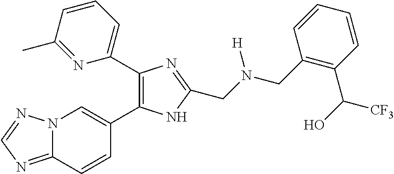 [SMILES: Cc1cccc(-c2nc(CNCc3ccccc3C(O)C(F)(F)F)[nH]c2-c2ccc3ncnn3c2)n1] |
| 29 | 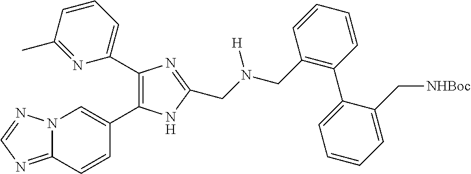 [SMILES: Cc1cccc(-c2nc(CNCc3ccccc3-c3ccccc3CNC(=O)OC(C)(C)C)[nH]c2-c2ccc3ncnn3c2)n1] |
| 30 | 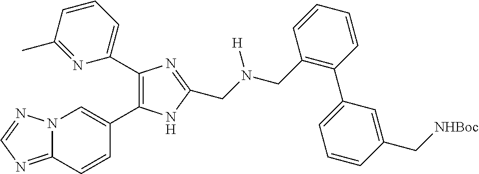 [SMILES: Cc1cccc(-c2nc(CNCc3ccccc3-c3cccc(CNC(=O)OC(C)(C)C)c3)[nH]c2-c2ccc3ncnn3c2)n1] |
| 31 | 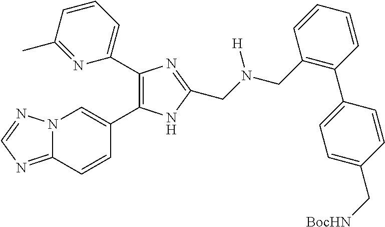 [SMILES: Cc1cccc(-c2nc(CNCc3ccccc3-c3ccc(CNC(=O)OC(C)(C)C)cc3)[nH]c2-c2ccc3ncnn3c2)n1] |
| 32 | 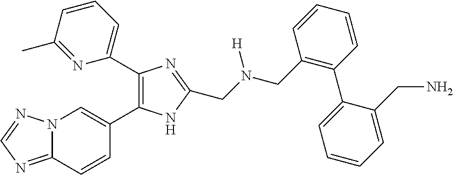 [SMILES: Cc1cccc(-c2nc(CNCc3ccccc3-c3ccccc3CN)[nH]c2-c2ccc3ncnn3c2)n1] |
| 33 | 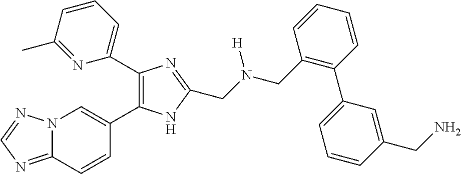 [SMILES: Cc1cccc(-c2nc(CNCc3ccccc3-c3cccc(CN)c3)[nH]c2-c2ccc3ncnn3c2)n1] |
| 34 | 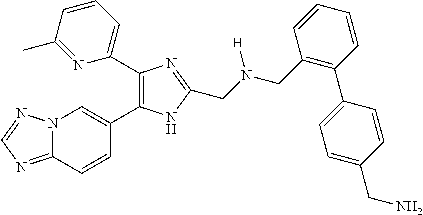 [SMILES: Cc1cccc(-c2nc(CNCc3ccccc3-c3ccc(CN)cc3)[nH]c2-c2ccc3ncnn3c2)n1] |
| 35 | 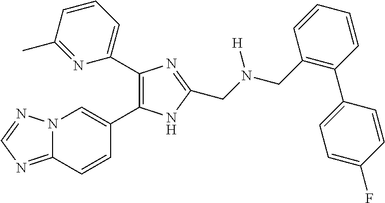 [SMILES: Cc1cccc(-c2nc(CNCc3ccccc3-c3ccc(F)cc3)[nH]c2-c2ccc3ncnn3c2)n1] |
| 36 | 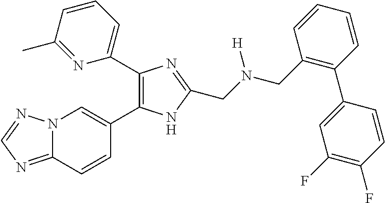 [SMILES: Cc1cccc(-c2nc(CNCc3ccccc3-c3ccc(F)c(F)c3)[nH]c2-c2ccc3ncnn3c2)n1] |
| 37 | 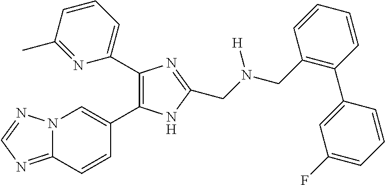 [SMILES: Cc1cccc(-c2nc(CNCc3ccccc3-c3cccc(F)c3)[nH]c2-c2ccc3ncnn3c2)n1] |
| 38 | 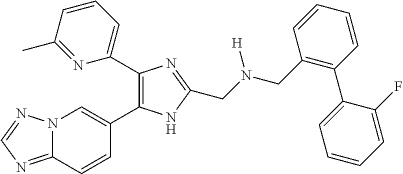 [SMILES: Cc1cccc(-c2nc(CNCc3ccccc3-c3ccccc3F)[nH]c2-c2ccc3ncnn3c2)n1] |
| 39 | 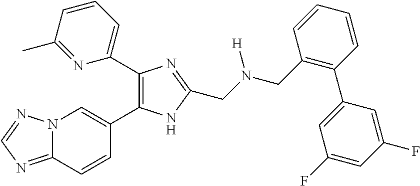 [SMILES: Cc1cccc(-c2nc(CNCc3ccccc3-c3cc(F)cc(F)c3)[nH]c2-c2ccc3ncnn3c2)n1] |
| 40 | 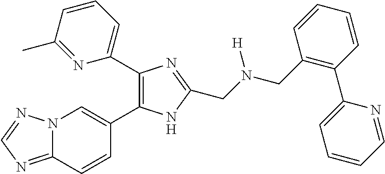 [SMILES: Cc1cccc(-c2nc(CNCc3ccccc3-c3ccccn3)[nH]c2-c2ccc3ncnn3c2)n1] |
| 41 | 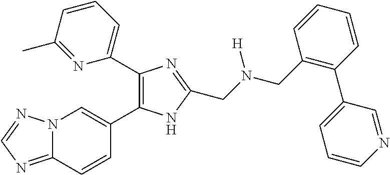 [SMILES: Cc1cccc(-c2nc(CNCc3ccccc3-c3cccnc3)[nH]c2-c2ccc3ncnn3c2)n1] |
| 42 | 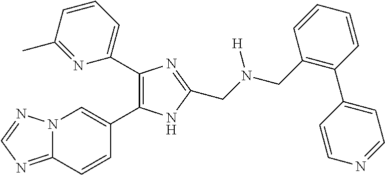 [SMILES: Cc1cccc(-c2nc(CNCc3ccccc3-c3ccncc3)[nH]c2-c2ccc3ncnn3c2)n1] |
| 43 | 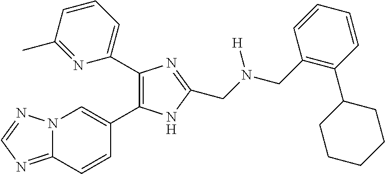 [SMILES: Cc1cccc(-c2nc(CNCc3ccccc3C3CCCCC3)[nH]c2-c2ccc3ncnn3c2)n1] |
| 44 | 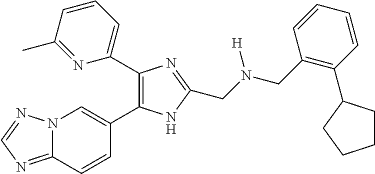 [SMILES: Cc1cccc(-c2nc(CNCc3ccccc3C3CCCC3)[nH]c2-c2ccc3ncnn3c2)n1] |
| 45 | 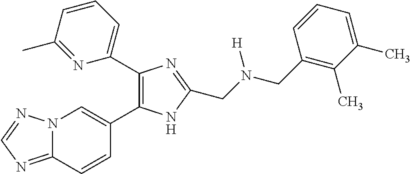 [SMILES: Cc1cccc(-c2nc(CNCc3cccc(C)c3C)[nH]c2-c2ccc3ncnn3c2)n1] |
| 46 | 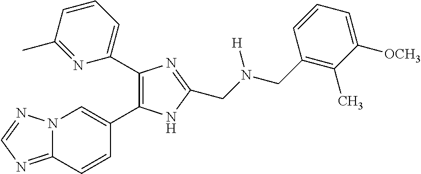 [SMILES: COc1cccc(CNCc2nc(-c3cccc(C)n3)c(-c3ccc4ncnn4c3)[nH]2)c1C] |
| 47 | 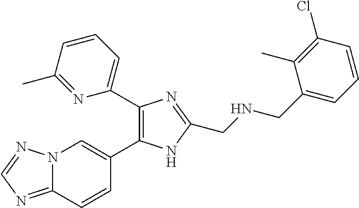 [SMILES: Cc1cccc(-c2nc(CNCc3cccc(Cl)c3C)[nH]c2-c2ccc3ncnn3c2)n1] |
| 48 | 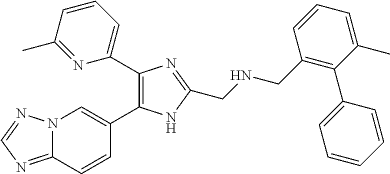 [SMILES: Cc1cccc(-c2nc(CNCc3cccc(C)c3-c3ccccc3)[nH]c2-c2ccc3ncnn3c2)n1] |
| 49 | 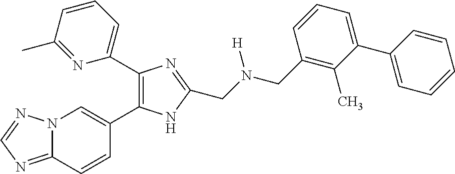 [SMILES: Cc1cccc(-c2nc(CNCc3cccc(-c4ccccc4)c3C)[nH]c2-c2ccc3ncnn3c2)n1] |
| 50 | 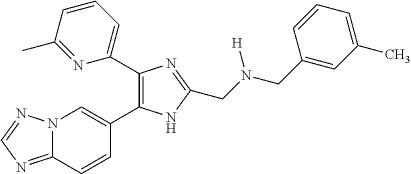 [SMILES: Cc1cccc(CNCc2nc(-c3cccc(C)n3)c(-c3ccc4ncnn4c3)[nH]2)c1] |
| 51 | 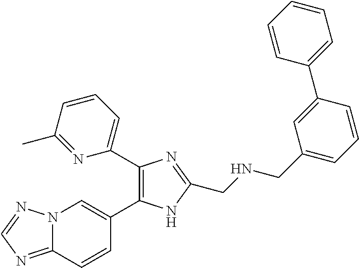 [SMILES: Cc1cccc(-c2nc(CNCc3cccc(-c4ccccc4)c3)[nH]c2-c2ccc3ncnn3c2)n1] |
| 52 | 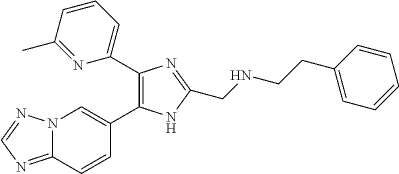 [SMILES: Cc1cccc(-c2nc(CNCCc3ccccc3)[nH]c2-c2ccc3ncnn3c2)n1] |
| 53 | 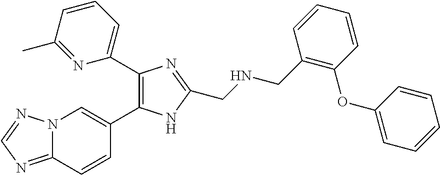 [SMILES: Cc1cccc(-c2nc(CNCc3ccccc3Oc3ccccc3)[nH]c2-c2ccc3ncnn3c2)n1] |
| 54 | 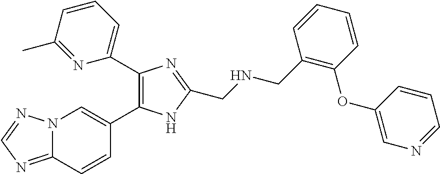 [SMILES: Cc1cccc(-c2nc(CNCc3ccccc3Oc3cccnc3)[nH]c2-c2ccc3ncnn3c2)n1] |
| 55 | 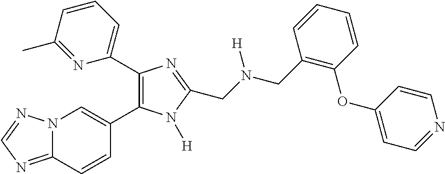 [SMILES: Cc1cccc(-c2nc(CNCc3ccccc3Oc3ccncc3)[nH]c2-c2ccc3ncnn3c2)n1] |
| 56 | 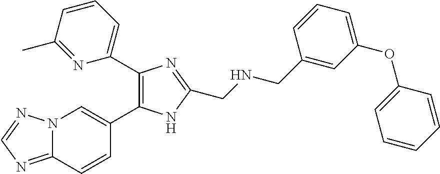 [SMILES: Cc1cccc(-c2nc(CNCc3cccc(Oc4ccccc4)c3)[nH]c2-c2ccc3ncnn3c2)n1] |
| 57 | 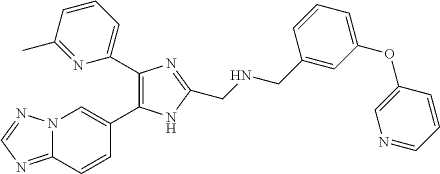 [SMILES: Cc1cccc(-c2nc(CNCc3cccc(Oc4cccnc4)c3)[nH]c2-c2ccc3ncnn3c2)n1] |
| 58 | 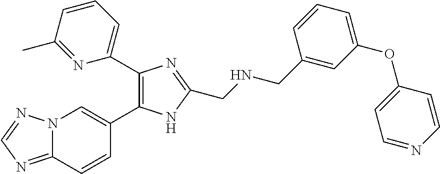 [SMILES: Cc1cccc(-c2nc(CNCc3cccc(Oc4ccncc4)c3)[nH]c2-c2ccc3ncnn3c2)n1] |
| 59 | 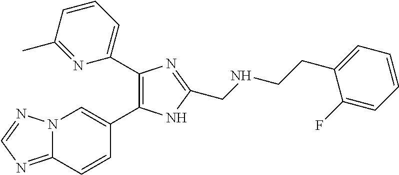 [SMILES: Cc1cccc(-c2nc(CNCCc3ccccc3F)[nH]c2-c2ccc3ncnn3c2)n1] |
| 60 | 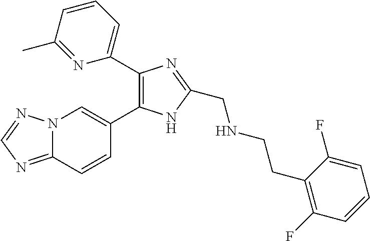 [SMILES: Cc1cccc(-c2nc(CNCCc3c(F)cccc3F)[nH]c2-c2ccc3ncnn3c2)n1] |
| 61 | 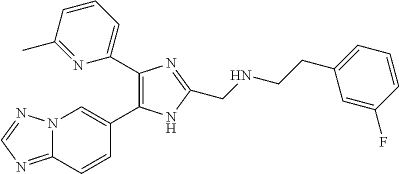 [SMILES: Cc1cccc(-c2nc(CNCCc3cccc(F)c3)[nH]c2-c2ccc3ncnn3c2)n1] |
| 62 | 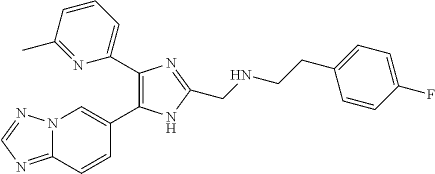 [SMILES: Cc1cccc(-c2nc(CNCCc3ccc(F)cc3)[nH]c2-c2ccc3ncnn3c2)n1] |
| 63 | 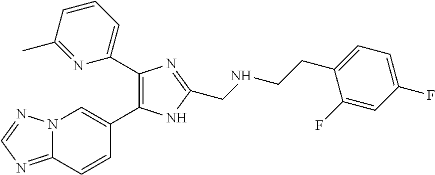 [SMILES: Cc1cccc(-c2nc(CNCCc3ccc(F)cc3F)[nH]c2-c2ccc3ncnn3c2)n1] |
| 64 | 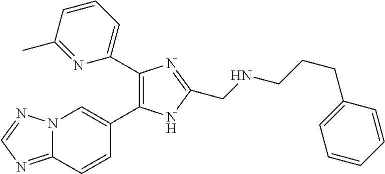 [SMILES: Cc1cccc(-c2nc(CNCCCc3ccccc3)[nH]c2-c2ccc3ncnn3c2)n1] |
| 65 | 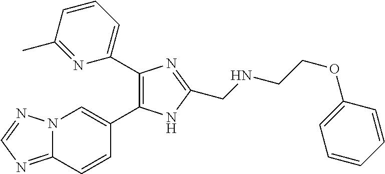 [SMILES: Cc1cccc(-c2nc(CNCCOc3ccccc3)[nH]c2-c2ccc3ncnn3c2)n1] |
| 66 | 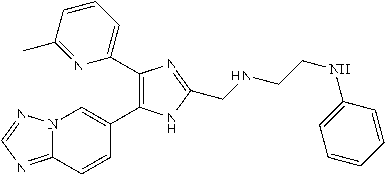 [SMILES: Cc1cccc(-c2nc(CNCCNc3ccccc3)[nH]c2-c2ccc3ncnn3c2)n1] |
| 67 | 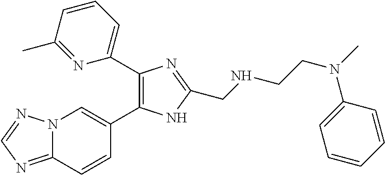 [SMILES: Cc1cccc(-c2nc(CNCCN(C)c3ccccc3)[nH]c2-c2ccc3ncnn3c2)n1] |
| 68 | 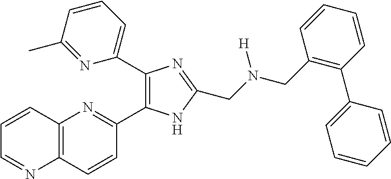 [SMILES: Cc1cccc(-c2nc(CNCc3ccccc3-c3ccccc3)[nH]c2-c2ccc3ncccc3n2)n1] |
| 69 | 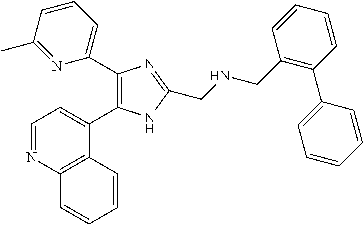 [SMILES: Cc1cccc(-c2nc(CNCc3ccccc3-c3ccccc3)[nH]c2-c2ccnc3ccccc23)n1] |
| 70 | 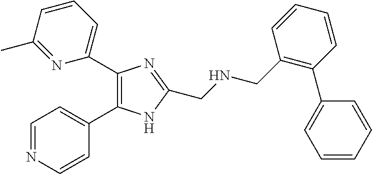 [SMILES: Cc1cccc(-c2nc(CNCc3ccccc3-c3ccccc3)[nH]c2-c2ccncc2)n1] |
| 71 | 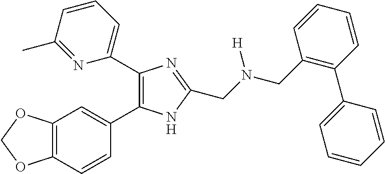 [SMILES: Cc1cccc(-c2nc(CNCc3ccccc3-c3ccccc3)[nH]c2-c2ccc3c(c2)OCO3)n1] |
| 72 | 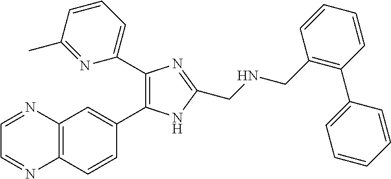 [SMILES: Cc1cccc(-c2nc(CNCc3ccccc3-c3ccccc3)[nH]c2-c2ccc3nccnc3c2)n1] |
| 73 | 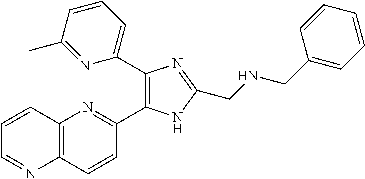 [SMILES: Cc1cccc(-c2nc(CNCc3ccccc3)[nH]c2-c2ccc3ncccc3n2)n1] |
| 74 | 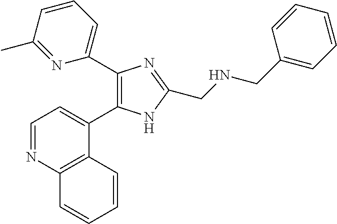 [SMILES: Cc1cccc(-c2nc(CNCc3ccccc3)[nH]c2-c2ccnc3ccccc23)n1] |
| 75 | 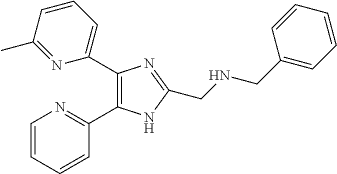 [SMILES: Cc1cccc(-c2nc(CNCc3ccccc3)[nH]c2-c2ccccn2)n1] |
| 76 | 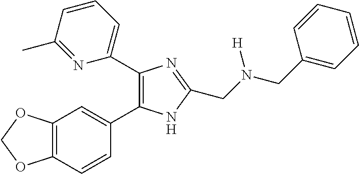 [SMILES: Cc1cccc(-c2nc(CNCc3ccccc3)[nH]c2-c2ccc3c(c2)OCO3)n1] |
| 77 | 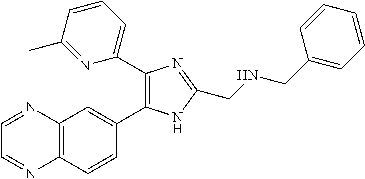 [SMILES: Cc1cccc(-c2nc(CNCc3ccccc3)[nH]c2-c2ccc3nccnc3c2)n1] |
| 78 | 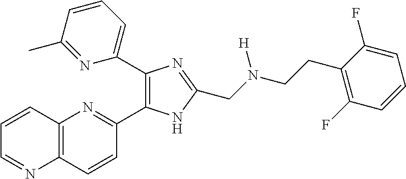 [SMILES: Cc1cccc(-c2nc(CNCCc3c(F)cccc3F)[nH]c2-c2ccc3ncccc3n2)n1] |
| 79 | 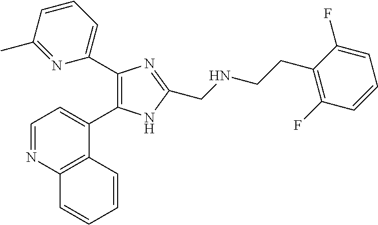 [SMILES: Cc1cccc(-c2nc(CNCCc3c(F)cccc3F)[nH]c2-c2ccnc3ccccc23)n1] |
| 80 | 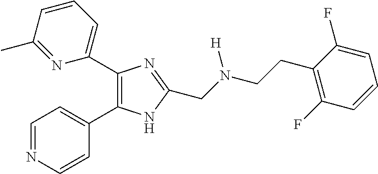 [SMILES: Cc1cccc(-c2nc(CNCCc3c(F)cccc3F)[nH]c2-c2ccncc2)n1] |
| 81 | 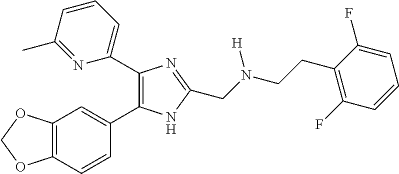 [SMILES: Cc1cccc(-c2nc(CNCCc3c(F)cccc3F)[nH]c2-c2ccc3c(c2)OCO3)n1] |
| 82 | 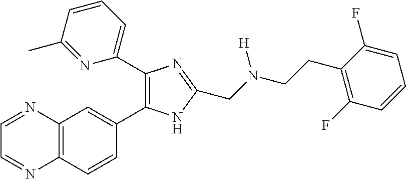 [SMILES: Cc1cccc(-c2nc(CNCCc3c(F)cccc3F)[nH]c2-c2ccc3nccnc3c2)n1] |
| 83 | 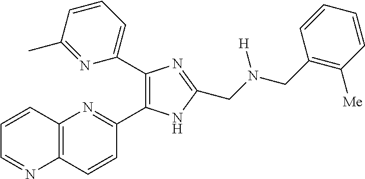 [SMILES: Cc1cccc(-c2nc(CNCc3ccccc3C)[nH]c2-c2ccc3ncccc3n2)n1] |
| 84 | 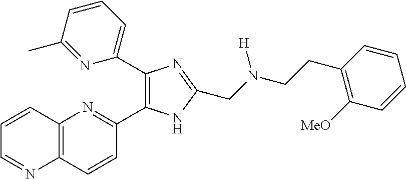 [SMILES: COc1ccccc1CCNCc1nc(-c2cccc(C)n2)c(-c2ccc3ncccc3n2)[nH]1] |
| 85 | 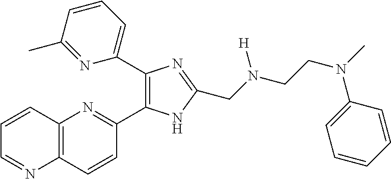 [SMILES: Cc1cccc(-c2nc(CNCCN(C)c3ccccc3)[nH]c2-c2ccc3ncccc3n2)n1] |
| 86 | 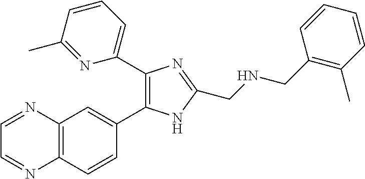 [SMILES: Cc1cccc(-c2nc(CNCc3ccccc3C)[nH]c2-c2ccc3nccnc3c2)n1] |
| 87 | 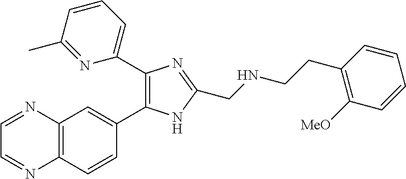 [SMILES: COc1ccccc1CCNCc1nc(-c2cccc(C)n2)c(-c2ccc3nccnc3c2)[nH]1] |
| 88 | 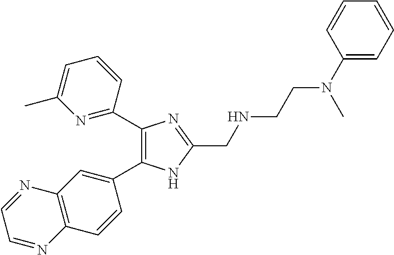 [SMILES: Cc1cccc(-c2nc(CNCCN(C)c3ccccc3)[nH]c2-c2ccc3nccnc3c2)n1] |
| 89 | 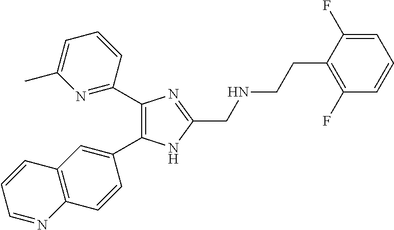 [SMILES: Cc1cccc(-c2nc(CNCCc3c(F)cccc3F)[nH]c2-c2ccc3ncccc3c2)n1] |
| 90 | 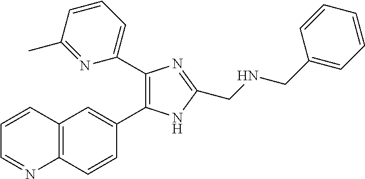 [SMILES: Cc1cccc(-c2nc(CNCc3ccccc3)[nH]c2-c2ccc3ncccc3c2)n1] |
| | and |
| 91 | 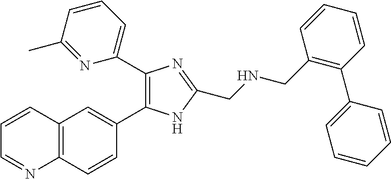 [SMILES: Cc1cccc(-c2nc(CNCc3ccccc3-c3ccccc3)[nH]c2-c2ccc3ncccc3c2)n1] |

and pharmaceutically acceptable salts thereof.

35. The conjugate of claim 1, wherein the TGFβR1 inhibitor is a compound of Formula (B-I): 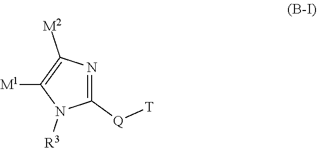
wherein:
M¹ and M² are independently selected from 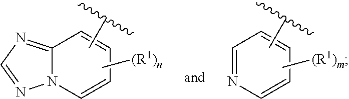
R¹ and R² are independently selected at each occurrence from:
a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, and —CN;
—C₁-C₁₀ alkyl, —C₂-C₁₀ alkenyl, and —C₂-C₁₀ alkynyl, each of which is optionally substituted at each occurrence with one or more substituents independently selected from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, a C₃-C₁₀ carbocycle, and a 3- to 10-membered heterocycle; and
a C₃-C₁₀ carbocycle and a 3- to 10-membered heterocycle, each of which is optionally substituted with one or more substituents independently selected from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, —C₁-C₆ alkyl, —C₂-C₆ alkenyl, and —C₂-C₆ alkynyl;
R³ is selected from hydrogen and —C₁-C₁₀ alkyl optionally substituted with one or more substituents independently selected from a halogen, —NO₂, ═O, ═S, ═N(R¹⁰), —CN, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, and —OC(O)R¹⁰;
n and m are independently selected from 0, 1, 2, 3, and 4;
Q is selected from a bond, —(CR¹⁰₂)ₚ—, —(CR¹⁰₂)_{q}C(═O)(CR¹⁰₂)_{q}—, —(CR¹⁰₂)_{q}C(═S)(CR¹⁰₂)_{q}—, —(CR¹⁰₂)_{q}C(═NR¹⁰)(CR¹⁰₂)_{q}—, —(CR¹⁰₂)_{q}O(CR¹⁰₂)_{q}—, —(CR¹⁰₂)_{q}S(CR¹⁰₂)_{q}—, —(CR¹⁰₂)_{q}N(R¹⁰)(CR¹⁰₂)_{q}—, —(CR¹⁰₂)_{q}OC(═O)O(CR¹⁰₂)_{q}—, —(CR¹⁰₂)_{q}C(═O)N(R¹⁰)(CR¹⁰₂)_{q}—, —(CR¹⁰₂)_{q}N(R¹⁰)C(═O)(CR¹⁰₂)_{q}—, and —(CR¹⁰₂)_{q}N(R¹⁰)SO₂(CR¹⁰₂)_{q}—;
p is selected from 1, 2, 3, 4, and 5;
q is independently selected at each occurrence from 0, 1, 2, 3, 4, and 5;
T is selected from an optionally substituted saturated C₃-C₇ carbocycle, an optionally substituted C₅₋₁₂ bicyclic carbocycle, and an optionally substituted 4- to 12-membered heterocycle, wherein T is optionally substituted with one or more substituents independently selected at each occurrence from R¹³;
R¹³ is independently selected at each occurrence from:
a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), and —CN;
—C₁-C₁₀ alkyl, —C₂-C₁₀ alkenyl, and —C₂-C₁₀ alkynyl, each of which is optionally substituted at each occurrence with one or more substituents independently selected from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, a C₃-C₁₀ carbocycle, and a 3- to 10-membered heterocycle; and
a C₃-C₁₀ carbocycle and a 3- to 10-membered heterocycle, each of which is optionally substituted with one or more substituents independently selected from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, —C₁-C₆ alkyl, —C₂-C₆ alkenyl, and —C₂-C₆ alkynyl; and
R¹⁰ is independently selected at each occurrence from:
hydrogen;
—C₁-C₁₀ alkyl, —C₂-C₁₀ alkenyl, and —C₂-C₁₀ alkynyl, each of which is optionally substituted at each occurrence with one or more substituents independently selected from a halogen, —OH, —CN, —NO₂, —NH₂, ═O, ═S, —O—C₁-C₁₀ alkyl, C₃-C₁₂ carbocycle, and a 3- to 12-membered heterocycle; and
a C₃-C₁₂ carbocycle and a 3- to 12-membered heterocycle, each of which is optionally substituted at each occurrence with one or more substituents independently selected from a halogen, —OH, —CN, —NO₂, —NH₂, ═O, ═S, —C₁-C₁₀ alkyl, —O—C₁-C₁₀ alkyl, and —C₁-C₁₀ haloalkyl;
or a salt thereof.

36.-38. (canceled)

39. The conjugate of claim 1, wherein the linker is covalently attached to the TGFββR1 inhibitor through a nitrogen of the inhibitor, and 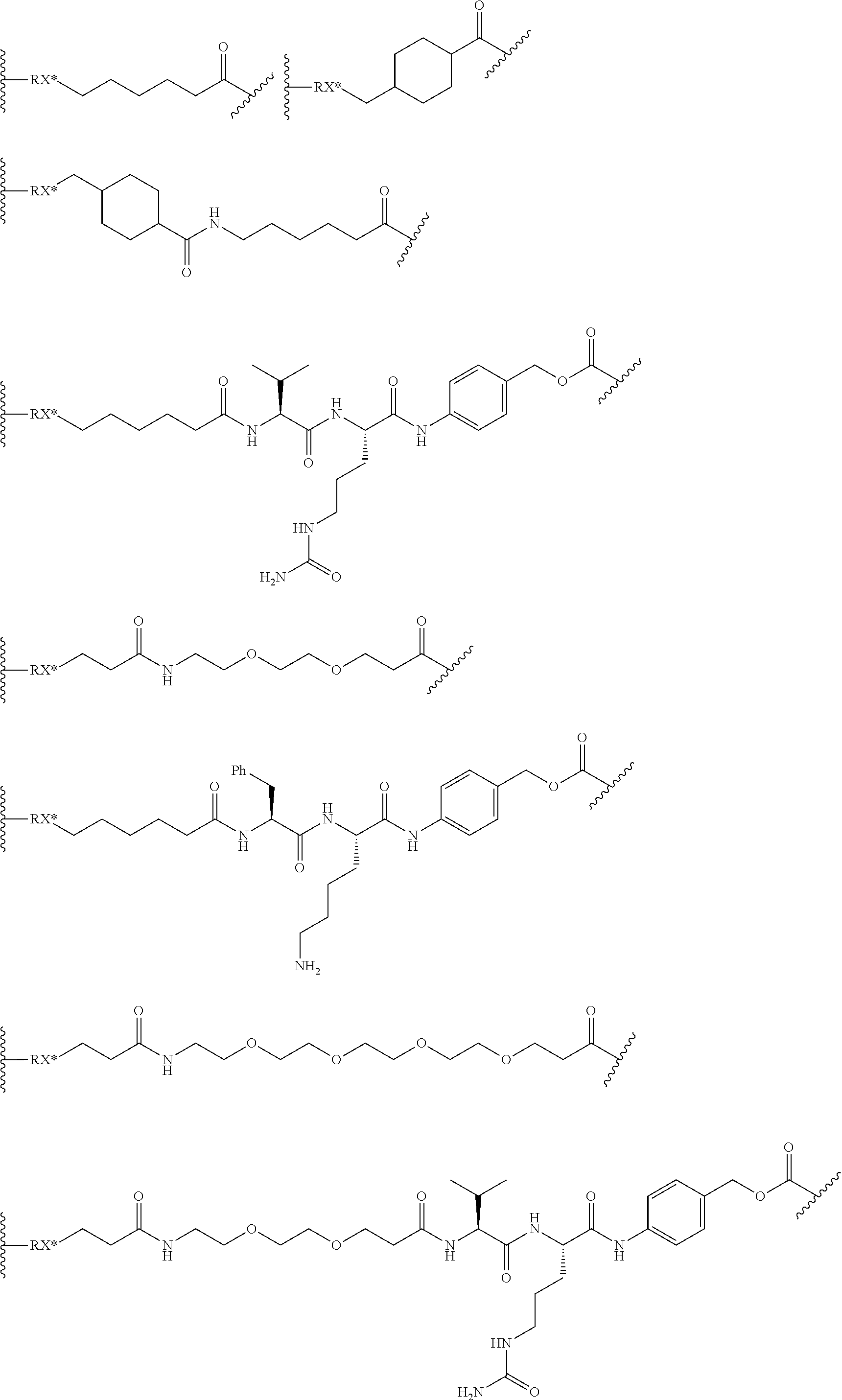 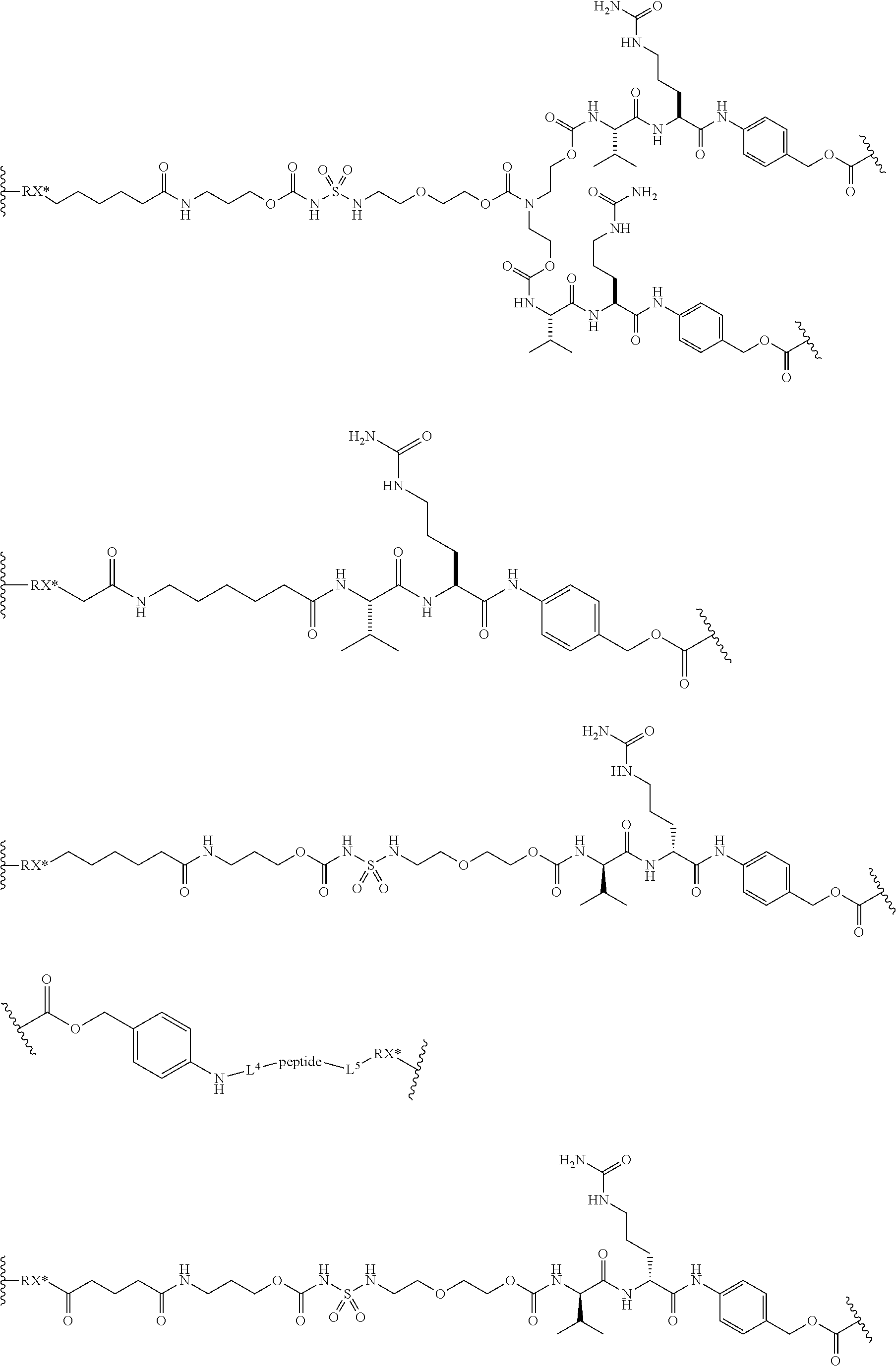 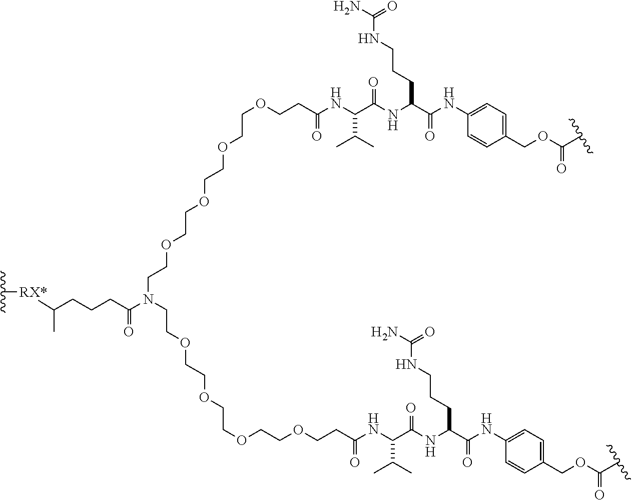
wherein 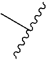
represents the point of attachment of the linker to the nitrogen of the TGFβR1 inhibitor;
RX* is a bond, a succinamide moiety, or a hydrolyzed succinamide moiety bound to a residue of the antibody construct, wherein 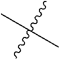
on RX* represents the point of attachment to the antibody construct; optionally wherein 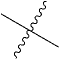
on RX* represents the point of attachment to a cysteine residue of the antibody construct;
L⁴ represents the C-terminus of the peptide; and
L⁵ is selected from a bond, alkylene, and heteroalkylene,
wherein L⁵ is optionally substituted with one or more groups independently selected from R³⁰;
wherein each R³⁰ is independently selected from halogen, —OH, —CN, —O-alkyl, —SH, ═O, ═S, —NH₂, —NO₂; and C₂-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂.

40. The conjugate of claim 1, wherein the linker is covalently attached to the TGFβR1 inhibitor through a nitrogen of the inhibitor, and the linker is selected from linkers IVb.1 to IVb.19.

41. The conjugate to claim 1, wherein the linker covalently attached to the TGFβR1 inhibitor comprises a structure of Table 2.

42. A pharmaceutical composition comprising the conjugate of claim 1 and a pharmaceutically acceptable carrier.

43. A method of treating a disease mediated by TGFβR1 activity in a subject in need thereof comprising administering to the subject an effective amount of a conjugate of a pharmaceutical composition of claim 42.

44. The method of claim 43, wherein the subject does not have cancer.

45. The method of claim 43, wherein the disease is liver cancer, or hepatocellular carcinoma.

46. The method of claim 43, wherein the disease is liver fibrosis.

47. The method of claim 46, wherein the liver fibrosis is associated with scleroderma, systemic fibrosis, steatohepatitis, non-alcoholic steatohepatitis (NASH), chronic HBV and HCV infection, autoimmune hepatitis, or primary biliary cholangitis.

48. The method of claim 43, wherein the conjugate is administered systemically.

49. The method of claim 43, wherein the conjugate is administered intravenously, cutaneously, or subcutaneously.

50. The method of claim 43, wherein the method further comprises administering a further active compound selected from nintedanib, pirfenidone, or both.

51.-52. (canceled)


SEQUENCE LISTING (249 sequences)

SEQ ID NO: 1 - protein, 5 aa, Artificial Sequence
  Synthetic sequence G2D VH CDR1
GYYMH

SEQ ID NO: 2 - protein, 5 aa, Artificial Sequence
  Synthetic sequence K2E VH CDR1
SYTMH

SEQ ID NO: 3 - protein, 6 aa, Artificial Sequence
  Synthetic sequence L4L VH CDR1
SDYAWN

SEQ ID NO: 4 - protein, 6 aa, Artificial Sequence
  Synthetic sequence H8K VH CDR1
SDYAWN

SEQ ID NO: 5 - protein, 5 aa, Artificial Sequence
  Synthetic sequence J4F VH CDR1
SYWIN

SEQ ID NO: 6 - protein, 17 aa, Artificial Sequence
  Synthetic sequence G2D VH CDR2
RINPNNGATNYNQNFKD

SEQ ID NO: 7 - protein, 17 aa, Artificial Sequence
  Synthetic sequence G2.1D VH CDR2
RINPNQGATNYNQNFKD

SEQ ID NO: 8 - protein, 17 aa, Artificial Sequence
  Synthetic sequence G2.2D VH CDR2
RINPNNAATNYNQNFKD

SEQ ID NO: 9 - protein, 17 aa, Artificial Sequence
  Synthetic sequence K2E VH CDR2
YISPSSGYTEYNQKFKD

SEQ ID NO: 10 - protein, 16 aa, Artificial Sequence
  Synthetic sequence L4L VH CDR2
YISYSGSTRYNPSLKS

SEQ ID NO: 11 - protein, 16 aa, Artificial Sequence
  Synthetic sequence H8K VH CDR2
YRSYRGSTSYNPSLKS

SEQ ID NO: 12 - protein, 17 aa, Artificial Sequence
  Synthetic sequence J4F VH CDR2
RIVPGSGSTYYNEMFKD

SEQ ID NO: 13 - protein, 5 aa, Artificial Sequence
  Synthetic sequence G2D VH CDR3
VNFYY

SEQ ID NO: 14 - protein, 4 aa, Artificial Sequence
  Synthetic sequence K2E VH CDR3
KFDY

SEQ ID NO: 15 - protein, 12 aa, Artificial Sequence
  Synthetic sequence L4L VH CDR3
RYRYDEGYGMDY

SEQ ID NO: 16 - protein, 14 aa, Artificial Sequence
  Synthetic sequence H8K VH CDR3
RGYYGSSSHWYFDV

SEQ ID NO: 17 - protein, 6 aa, Artificial Sequence
  Synthetic sequence J4F VH CDR3
KPNFDV

SEQ ID NO: 18 - protein, 11 aa, Artificial Sequence
  Synthetic sequence G2D VL CDR1
KASQVINSYLS

SEQ ID NO: 19 - protein, 11 aa, Artificial Sequence
  Synthetic sequence K2E VL CDR1
KASQDINSYLS

SEQ ID NO: 20 - protein, 11 aa, Artificial Sequence
  Synthetic sequence L4L VL CDR1
RASENIYSNLA

SEQ ID NO: 21 - protein, 11 aa, Artificial Sequence
  Synthetic sequence H8K VL CDR1
RASENIYSYLA

SEQ ID NO: 22 - protein, 11 aa, Artificial Sequence
  Synthetic sequence J4F VL CDR1
KASQNVGTNVA

SEQ ID NO: 23 - protein, 7 aa, Artificial Sequence
  Synthetic sequence G2D VL CDR2
RANTLVD

SEQ ID NO: 24 - protein, 7 aa, Artificial Sequence
  Synthetic sequence G2.1D VL CDR2
RANTLVS

SEQ ID NO: 25 - protein, 7 aa, Artificial Sequence
  Synthetic sequence G2.2D VL CDR2
RANTLVE

SEQ ID NO: 26 - protein, 7 aa, Artificial Sequence
  Synthetic sequence K2E VL CDR2
RANRLVD

SEQ ID NO: 27 - protein, 7 aa, Artificial Sequence
  Synthetic sequence K2.1E VL CDR2
RANRLVE

SEQ ID NO: 28 - protein, 7 aa, Artificial Sequence
  Synthetic sequence L4L VL CDR2
AATNLAD

SEQ ID NO: 29 - protein, 7 aa, Artificial Sequence
  Synthetic sequence L4.1L VL CDR2
AATNLAS

SEQ ID NO: 30 - protein, 7 aa, Artificial Sequence
  Synthetic sequence L4.2L VL CDR2
AATNLAE

SEQ ID NO: 31 - protein, 7 aa, Artificial Sequence
  Synthetic sequence H8K VL CDR2
NAKTLAE

SEQ ID NO: 32 - protein, 7 aa, Artificial Sequence
  Synthetic sequence J4F VL CDR2
SASYRFS

SEQ ID NO: 33 - protein, 9 aa, Artificial Sequence
  Synthetic sequence G2D VL CDR3
LQYAEFPYT

SEQ ID NO: 34 - protein, 9 aa, Artificial Sequence
  Synthetic sequence K2E VL CDR3
LQYDEFPFT

SEQ ID NO: 35 - protein, 10 aa, Artificial Sequence
  Synthetic sequence L4L VL CDR3
QHFWGTPPWT

SEQ ID NO: 36 - protein, 9 aa, Artificial Sequence
  Synthetic sequence H8K VL CDR3
QHHYGTPLT

SEQ ID NO: 37 - protein, 9 aa, Artificial Sequence
  Synthetic sequence J4F VL CDR3
QQYNSYPLT

SEQ ID NO: 38 - protein, 114 aa, Artificial Sequence
  Synthetic sequence G2D VH
EVQLQQSGPELVKPGASVKISCKASGYSFTGYYMHWVKQSHVKSLEWIGRINPNNGATNYNQNFKDKASLTVDKSSSTAYMEFHSLTSEDSAVYYCTSVNFYYWGQGTTLTVSS

SEQ ID NO: 39 - protein, 113 aa, Artificial Sequence
  Synthetic sequence K2E VH
QVQLQQSAAELARPGASVKMSCKASGYTFTSYTMHWVKQRPGQGLEWIGYISPSSGYTEYNQKFKDKTTLTADKSSSTAYMQLSSLTSEDSAVYYCARKFDYWGQGTTLTVSS

SEQ ID NO: 40 - protein, 121 aa, Artificial Sequence
  Synthetic sequence L4L VH
DVQLQESGPGLVKPSQSLSLTCTVTGYSITSDYAWNWIRQFPGNKLEWMGYISYSGSTRYNPSLKSRISITRDTSKNQFFLQLNSVTTEDTATYYCARRYRYDEGYGMDYWGQGTSVTVSS

SEQ ID NO: 41 - protein, 123 aa, Artificial Sequence
  Synthetic sequence H8K VH
DVQLQESGPGLVKPSQSLSLTCTVTGFSITSDYAWNWIRQFPGNQLEWMGYRSYRGSTSYNPSLKSRISITRDTSKNQFFLQLNSVTTEDTATYYCARRGYYGSSSHWYFDVWGAGTTVTVSS

SEQ ID NO: 42 - protein, 115 aa, Artificial Sequence
  Synthetic sequence J4F VH
QVQLQQSGDDLVKPGASVKLSCKASGYTFTSYWINWIKQRPGQGLEWIGRIVPGSGSTYYNEMFKDKATLTVDTSSSTAYIQLSSLSSEDSAVYFCARKPNFDVWGAGTTVTVSS

SEQ ID NO: 43 - protein, 114 aa, Artificial Sequence
  Synthetic sequence hzG2D VH V109L
QVQLVQSGAEVKKPGASVKVSCKASGYTFTGYYMHWVRQAPGQRLEWMGRINPNNGATNYNQNFKDRVTITRDTSASTAYMELSSLRSEDTAVYYCARVNFYYWGQGTTLTVSS

SEQ ID NO: 44 - protein, 114 aa, Artificial Sequence
  Synthetic sequence hzG2.1D VH T28S, V109L
QVQLVQSGAEVKKPGASVKVSCKASGYSFTGYYMHWVRQAPGQRLEWMGRINPNNGATNYNQNFKDRVTITRDTSASTAYMELSSLRSEDTAVYYCARVNFYYWGQGTTLTVSS

SEQ ID NO: 45 - protein, 114 aa, Artificial Sequence
  Synthetic sequence hzG2.2D VH T28S, R66K, V67A, V109L
QVQLVQSGAEVKKPGASVKVSCKASGYSFTGYYMHWVRQAPGQRLEWMGRINPNNGATNYNQNFKDKATITRDTSASTAYMELSSLRSEDTAVYYCARVNFYYWGQGTTLTVSS

SEQ ID NO: 46 - protein, 114 aa, Artificial Sequence
  Synthetic sequence hzG2.3D VH T28S, R66K, V67A, A93T, R94S, V109L
QVQLVQSGAEVKKPGASVKVSCKASGYSFTGYYMHWVRQAPGQRLEWMGRINPNNGATNYNQNFKDKATITRDTSASTAYMELSSLRSEDTAVYYCTSVNFYYWGQGTTLTVSS

SEQ ID NO: 47 - protein, 114 aa, Artificial Sequence
  Synthetic sequence hzG2.4D VH T28S, R66K, V67A, T68S, I69L, R71V, V109L
QVQLVQSGAEVKKPGASVKVSCKASGYSFTGYYMHWVRQAPGQRLEWMGRINPNNGATNYNQNFKDKASLTVDTSASTAYMELSSLRSEDTAVYYCARVNFYYWGQGTTLTVSS

SEQ ID NO: 48 - protein, 114 aa, Artificial Sequence
  Synthetic sequence hzG2.5D VH T28S, R66K, V67A, T68S, I69L, R71V, A93T, R94S, V109L
QVQLVQSGAEVKKPGASVKVSCKASGYSFTGYYMHWVRQAPGQRLEWMGRINPNNGATNYNQNFKDKASLTVDTSASTAYMELSSLRSEDTAVYYCTSVNFYYWGQGTTLTVSS

SEQ ID NO: 49 - protein, 114 aa, Artificial Sequence
  Synthetic sequence hzG2.6D VH T28S, T68S, I69L, R71V, V109L
QVQLVQSGAEVKKPGASVKVSCKASGYSFTGYYMHWVRQAPGQRLEWMGRINPNNGATNYNQNFKDRVSLTVDTSASTAYMELSSLRSEDTAVYYCARVNFYYWGQGTTLTVSS

SEQ ID NO: 50 - protein, 114 aa, Artificial Sequence
  Synthetic sequence hzG2.7D VH T28S, T68S, I69L, R71V, A93T, R94S, V109L
QVQLVQSGAEVKKPGASVKVSCKASGYSFTGYYMHWVRQAPGQRLEWMGRINPNNGATNYNQNFKDRVSLTVDTSASTAYMELSSLRSEDTAVYYCTSVNFYYWGQGTTLTVSS

SEQ ID NO: 51 - protein, 114 aa, Artificial Sequence
  Synthetic sequence hzG2.8D VH T28S, A93T, R94S, V109L
QVQLVQSGAEVKKPGASVKVSCKASGYSFTGYYMHWVRQAPGQRLEWMGRINPNNGATNYNQNFKDRVTITRDTSASTAYMELSSLRSEDTAVYYCTSVNFYYWGQGTTLTVSS

SEQ ID NO: 52 - protein, 114 aa, Artificial Sequence
  Synthetic sequence hzG2.9D VH T28S, N54Q
QVQLVQSGAEVKKPGASVKVSCKASGYSFTGYYMHWVRQAPGQRLEWMGRINPNQGATNYNQNFKDRVTITRDTSASTAYMELSSLRSEDTAVYYCARVNFYYWGQGTTLTVSS

SEQ ID NO: 53 - protein, 114 aa, Artificial Sequence
  Synthetic sequence hzG2.10D VH T28S, N54Q, R66K, V67A
QVQLVQSGAEVKKPGASVKVSCKASGYSFTGYYMHWVRQAPGQRLEWMGRINPNQGATNYNQNFKDKATITRDTSASTAYMELSSLRSEDTAVYYCARVNFYYWGQGTTLTVSS

SEQ ID NO: 54 - protein, 114 aa, Artificial Sequence
  Synthetic sequence hzG2.11D VH T28S, N54Q, R66K, V67A, A93T, R94S
QVQLVQSGAEVKKPGASVKVSCKASGYSFTGYYMHWVRQAPGQRLEWMGRINPNQGATNYNQNFKDKATITRDTSASTAYMELSSLRSEDTAVYYCTSVNFYYWGQGTTLTVSS

SEQ ID NO: 55 - protein, 114 aa, Artificial Sequence
  Synthetic sequence hzG2.12D VH T28S, N54Q, R66K, V67A, T68S, I69L, R71V
QVQLVQSGAEVKKPGASVKVSCKASGYSFTGYYMHWVRQAPGQRLEWMGRINPNQGATNYNQNFKDKASLTVDTSASTAYMELSSLRSEDTAVYYCARVNFYYWGQGTTLTVSS

SEQ ID NO: 56 - protein, 114 aa, Artificial Sequence
  Synthetic sequence hzG2.13D VH T28S, N54Q, R66K, V67A, T68S, I69L, R71V, A93T, R94S
QVQLVQSGAEVKKPGASVKVSCKASGYSFTGYYMHWVRQAPGQRLEWMGRINPNQGATNYNQNFKDKASLTVDTSASTAYMELSSLRSEDTAVYYCTSVNFYYWGQGTTLTVSS

SEQ ID NO: 57 - protein, 114 aa, Artificial Sequence
  Synthetic sequence hzG2.14D VH T28S, N54Q, T68S, I69L, R71V
QVQLVQSGAEVKKPGASVKVSCKASGYSFTGYYMHWVRQAPGQRLEWMGRINPNQGATNYNQNFKDRVSLTVDTSASTAYMELSSLRSEDTAVYYCARVNFYYWGQGTTLTVSS

SEQ ID NO: 58 - protein, 114 aa, Artificial Sequence
  Synthetic sequence hzG2.15D VH T28S, N54Q, T68S, I69L, R71V, A93T, R94S
QVQLVQSGAEVKKPGASVKVSCKASGYSFTGYYMHWVRQAPGQRLEWMGRINPNQGATNYNQNFKDRVSLTVDTSASTAYMELSSLRSEDTAVYYCTSVNFYYWGQGTTLTVSS

SEQ ID NO: 59 - protein, 114 aa, Artificial Sequence
  Synthetic sequence hzG2.16D VH T28S, N54Q, A93T, R94S
QVQLVQSGAEVKKPGASVKVSCKASGYSFTGYYMHWVRQAPGQRLEWMGRINPNQGATNYNQNFKDRVTITRDTSASTAYMELSSLRSEDTAVYYCTSVNFYYWGQGTTLTVSS

SEQ ID NO: 60 - protein, 114 aa, Artificial Sequence
  Synthetic sequence hzG2.17D VH T28S, G55A
QVQLVQSGAEVKKPGASVKVSCKASGYSFTGYYMHWVRQAPGQRLEWMGRINPNNAATNYNQNFKDRVTITRDTSASTAYMELSSLRSEDTAVYYCARVNFYYWGQGTTLTVSS

SEQ ID NO: 61 - protein, 114 aa, Artificial Sequence
  Synthetic sequence hzG2.18D VH T28S, G55A, R66K, V67A
QVQLVQSGAEVKKPGASVKVSCKASGYSFTGYYMHWVRQAPGQRLEWMGRINPNNAATNYNQNFKDKATITRDTSASTAYMELSSLRSEDTAVYYCARVNFYYWGQGTTLTVSS

SEQ ID NO: 62 - protein, 114 aa, Artificial Sequence
  Synthetic sequence hzG2.19D VH T28S, G55A, R66K, V67A, A93T, R94S
QVQLVQSGAEVKKPGASVKVSCKASGYSFTGYYMHWVRQAPGQRLEWMGRINPNNAATNYNQNFKDKATITRDTSASTAYMELSSLRSEDTAVYYCTSVNFYYWGQGTTLTVSS

SEQ ID NO: 63 - protein, 114 aa, Artificial Sequence
  Synthetic sequence hzG2.20D VH T28S, G55A, R66K, V67A, T68S, I69L, R71V
QVQLVQSGAEVKKPGASVKVSCKASGYSFTGYYMHWVRQAPGQRLEWMGRINPNNAATNYNQNFKDKASLTVDTSASTAYMELSSLRSEDTAVYYCARVNFYYWGQGTTLTVSS

SEQ ID NO: 64 - protein, 114 aa, Artificial Sequence
  Synthetic sequence hzG2.21D VH T28S, G55A, R66K, V67A, T68S, I69L, R71V, A93T, R94S
QVQLVQSGAEVKKPGASVKVSCKASGYSFTGYYMHWVRQAPGQRLEWMGRINPNNAATNYNQNFKDKASLTVDTSASTAYMELSSLRSEDTAVYYCTSVNFYYWGQGTTLTVSS

SEQ ID NO: 65 - protein, 114 aa, Artificial Sequence
  Synthetic sequence hzG2.22D VH T28S, G55A, T68S, I69L, R71V
QVQLVQSGAEVKKPGASVKVSCKASGYSFTGYYMHWVRQAPGQRLEWMGRINPNNAATNYNQNFKDRVSLTVDTSASTAYMELSSLRSEDTAVYYCARVNFYYWGQGTTLTVSS

SEQ ID NO: 66 - protein, 114 aa, Artificial Sequence
  Synthetic sequence hzG2.23D VH T28S, G55A, T68S, I69L, R71V, A93T, R94S
QVQLVQSGAEVKKPGASVKVSCKASGYSFTGYYMHWVRQAPGQRLEWMGRINPNNAATNYNQNFKDRVSLTVDTSASTAYMELSSLRSEDTAVYYCTSVNFYYWGQGTTLTVSS

SEQ ID NO: 67 - protein, 114 aa, Artificial Sequence
  Synthetic sequence hzG2.24D VH T28S, G55A, A93T, R94S
QVQLVQSGAEVKKPGASVKVSCKASGYSFTGYYMHWVRQAPGQRLEWMGRINPNNAATNYNQNFKDRVTITRDTSASTAYMELSSLRSEDTAVYYCTSVNFYYWGQGTTLTVSS

SEQ ID NO: 68 - protein, 114 aa, Artificial Sequence
  Synthetic sequence hzG2.25D VH N54Q, R66K, V67A
QVQLVQSGAEVKKPGASVKVSCKASGYTFTGYYMHWVRQAPGQRLEWMGRINPNQGATNYNQNFKDKATITRDTSASTAYMELSSLRSEDTAVYYCARVNFYYWGQGTTLTVSS

SEQ ID NO: 69 - protein, 114 aa, Artificial Sequence
  Synthetic sequence hzG2.26D VH N54Q, R66K, V67A, A93T, R94S
QVQLVQSGAEVKKPGASVKVSCKASGYTFTGYYMHWVRQAPGQRLEWMGRINPNQGATNYNQNFKDKATITRDTSASTAYMELSSLRSEDTAVYYCTSVNFYYWGQGTTLTVSS

SEQ ID NO: 70 - protein, 114 aa, Artificial Sequence
  Synthetic sequence hzG2.27D VH N54Q, R66K, V67A, T68S, I69L, R71V
QVQLVQSGAEVKKPGASVKVSCKASGYTFTGYYMHWVRQAPGQRLEWMGRINPNQGATNYNQNFKDKASLTVDTSASTAYMELSSLRSEDTAVYYCARVNFYYWGQGTTLTVSS

SEQ ID NO: 71 - protein, 114 aa, Artificial Sequence
  Synthetic sequence hzG2.28D VH N54Q, R66K, V67A, T68S, I69L, R71V, A93T, R94S
QVQLVQSGAEVKKPGASVKVSCKASGYTFTGYYMHWVRQAPGQRLEWMGRINPNQGATNYNQNFKDKASLTVDTSASTAYMELSSLRSEDTAVYYCTSVNFYYWGQGTTLTVSS

SEQ ID NO: 72 - protein, 114 aa, Artificial Sequence
  Synthetic sequence hzG2.29D VH N54Q, T68S, I69L, R71V
QVQLVQSGAEVKKPGASVKVSCKASGYTFTGYYMHWVRQAPGQRLEWMGRINPNQGATNYNQNFKDRVSLTVDTSASTAYMELSSLRSEDTAVYYCARVNFYYWGQGTTLTVSS

SEQ ID NO: 73 - protein, 114 aa, Artificial Sequence
  Synthetic sequence hzG2.30D VH N54Q, T68S, I69L, R71V, A93T, R94S
QVQLVQSGAEVKKPGASVKVSCKASGYTFTGYYMHWVRQAPGQRLEWMGRINPNQGATNYNQNFKDRVSLTVDTSASTAYMELSSLRSEDTAVYYCTSVNFYYWGQGTTLTVSS

SEQ ID NO: 74 - protein, 114 aa, Artificial Sequence
  Synthetic sequence hzG2.31D VH N54Q, A93T, R94S
QVQLVQSGAEVKKPGASVKVSCKASGYTFTGYYMHWVRQAPGQRLEWMGRINPNQGATNYNQNFKDRVTITRDTSASTAYMELSSLRSEDTAVYYCTSVNFYYWGQGTTLTVSS

SEQ ID NO: 75 - protein, 114 aa, Artificial Sequence
  Synthetic sequence hzG2.32D VH G55A, R66K, V67A
QVQLVQSGAEVKKPGASVKVSCKASGYTFTGYYMHWVRQAPGQRLEWMGRINPNNAATNYNQNFKDKATITRDTSASTAYMELSSLRSEDTAVYYCARVNFYYWGQGTTLTVSS

SEQ ID NO: 76 - protein, 114 aa, Artificial Sequence
  Synthetic sequence hzG2.33D VH G55A, R66K, V67A, A93T, R94S
QVQLVQSGAEVKKPGASVKVSCKASGYTFTGYYMHWVRQAPGQRLEWMGRINPNNAATNYNQNFKDKATITRDTSASTAYMELSSLRSEDTAVYYCTSVNFYYWGQGTTLTVSS

SEQ ID NO: 77 - protein, 114 aa, Artificial Sequence
  Synthetic sequence hzG2.34D VH G55A, R66K, V67A, T68S, I69L, R71V
QVQLVQSGAEVKKPGASVKVSCKASGYTFTGYYMHWVRQAPGQRLEWMGRINPNNAATNYNQNFKDKASLTVDTSASTAYMELSSLRSEDTAVYYCARVNFYYWGQGTTLTVSS

SEQ ID NO: 78 - protein, 114 aa, Artificial Sequence
  Synthetic sequence hzG2.35D VH G55A, R66K, V67A, T68S, I69L, R71V, A93T, R94S
QVQLVQSGAEVKKPGASVKVSCKASGYTFTGYYMHWVRQAPGQRLEWMGRINPNNAATNYNQNFKDKASLTVDTSASTAYMELSSLRSEDTAVYYCTSVNFYYWGQGTTLTVSS

SEQ ID NO: 79 - protein, 114 aa, Artificial Sequence
  Synthetic sequence hzG2.36D VH G55A, T68S, I69L, R71V
QVQLVQSGAEVKKPGASVKVSCKASGYTFTGYYMHWVRQAPGQRLEWMGRINPNNAATNYNQNFKDRVSLTVDTSASTAYMELSSLRSEDTAVYYCARVNFYYWGQGTTLTVSS

SEQ ID NO: 80 - protein, 114 aa, Artificial Sequence
  Synthetic sequence hzG2.37D VH G55A, T68S, I69L, R71V, A93T, R94S
QVQLVQSGAEVKKPGASVKVSCKASGYTFTGYYMHWVRQAPGQRLEWMGRINPNNAATNYNQNFKDRVSLTVDTSASTAYMELSSLRSEDTAVYYCTSVNFYYWGQGTTLTVSS

SEQ ID NO: 81 - protein, 114 aa, Artificial Sequence
  Synthetic sequence hzG2.38D VH G55A, A93T, R94S
QVQLVQSGAEVKKPGASVKVSCKASGYTFTGYYMHWVRQAPGQRLEWMGRINPNNAATNYNQNFKDRVTITRDTSASTAYMELSSLRSEDTAVYYCTSVNFYYWGQGTTLTVSS

SEQ ID NO: 82 - protein, 114 aa, Artificial Sequence
  Synthetic sequence hzG2.39D VH R66K, V67A, V109L
QVQLVQSGAEVKKPGASVKVSCKASGYTFTGYYMHWVRQAPGQRLEWMGRINPNNGATNYNQNFKDKATITRDTSASTAYMELSSLRSEDTAVYYCARVNFYYWGQGTTLTVSS

SEQ ID NO: 83 - protein, 114 aa, Artificial Sequence
  Synthetic sequence hzG2.40D VH R66K, V67A, A93T, R94S, V109L
QVQLVQSGAEVKKPGASVKVSCKASGYTFTGYYMHWVRQAPGQRLEWMGRINPNNGATNYNQNFKDKATITRDTSASTAYMELSSLRSEDTAVYYCTSVNFYYWGQGTTLTVSS

SEQ ID NO: 84 - protein, 114 aa, Artificial Sequence
  Synthetic sequence hzG2.41D VH R66K, V67A, T68S, I69L, R71V, V109L
QVQLVQSGAEVKKPGASVKVSCKASGYTFTGYYMHWVRQAPGQRLEWMGRINPNNGATNYNQNFKDKASLTVDTSASTAYMELSSLRSEDTAVYYCARVNFYYWGQGTTLTVSS

SEQ ID NO: 85 - protein, 114 aa, Artificial Sequence
  Synthetic sequence hzG2.42D VH R66K, V67A, T68S, I69L, R71V, A93T, R94S, V109L
QVQLVQSGAEVKKPGASVKVSCKASGYTFTGYYMHWVRQAPGQRLEWMGRINPNNGATNYNQNFKDKASLTVDTSASTAYMELSSLRSEDTAVYYCTSVNFYYWGQGTTLTVSS

SEQ ID NO: 86 - protein, 114 aa, Artificial Sequence
  Synthetic sequence hzG2.43D VH T68S, I69L, R71V, V109L
QVQLVQSGAEVKKPGASVKVSCKASGYTFTGYYMHWVRQAPGQRLEWMGRINPNNGATNYNQNFKDRVSLTVDTSASTAYMELSSLRSEDTAVYYCARVNFYYWGQGTTLTVSS

SEQ ID NO: 87 - protein, 114 aa, Artificial Sequence
  Synthetic sequence hzG2.44D VH T68S, I69L, R71V, A93T, R94S, V109L
QVQLVQSGAEVKKPGASVKVSCKASGYTFTGYYMHWVRQAPGQRLEWMGRINPNNGATNYNQNFKDRVSLTVDTSASTAYMELSSLRSEDTAVYYCTSVNFYYWGQGTTLTVSS

SEQ ID NO: 88 - protein, 114 aa, Artificial Sequence
  Synthetic sequence hzG2.45D VH A93T, R94S, V109L
QVQLVQSGAEVKKPGASVKVSCKASGYTFTGYYMHWVRQAPGQRLEWMGRINPNNGATNYNQNFKDRVTITRDTSASTAYMELSSLRSEDTAVYYCTSVNFYYWGQGTTLTVSS

SEQ ID NO: 89 - protein, 113 aa, Artificial Sequence
  Synthetic sequence hzK2E VH
QVQLVQSGAEVKKPGASVKVSCKASGYTFTSYTMHWVRQAPGQGLEWMGYISPSSGYTEYNQKFKDRVTMTRDTSTSTVYMELSSLRSEDTAVYYCARKFDYWGQGTTVTVSS

SEQ ID NO: 90 - protein, 113 aa, Artificial Sequence
  Synthetic sequence hzK2.1E VH R66K, V67T
QVQLVQSGAEVKKPGASVKVSCKASGYTFTSYTMHWVRQAPGQGLEWMGYISPSSGYTEYNQKFKDKTTMTRDTSTSTVYMELSSLRSEDTAVYYCARKFDYWGQGTTVTVSS

SEQ ID NO: 91 - protein, 113 aa, Artificial Sequence
  Synthetic sequence hzK2.2E VH R66K, V67T, M69L, R71A
QVQLVQSGAEVKKPGASVKVSCKASGYTFTSYTMHWVRQAPGQGLEWMGYISPSSGYTEYNQKFKDKTTLTADTSTSTVYMELSSLRSEDTAVYYCARKFDYWGQGTTVTVSS

SEQ ID NO: 92 - protein, 113 aa, Artificial Sequence
  Synthetic sequence hzK2.3E VH R66K, V67T, M69L, R71A, T73K
QVQLVQSGAEVKKPGASVKVSCKASGYTFTSYTMHWVRQAPGQGLEWMGYISPSSGYTEYNQKFKDKTTLTADKSTSTVYMELSSLRSEDTAVYYCARKFDYWGQGTTVTVSS

SEQ ID NO: 93 - protein, 113 aa, Artificial Sequence
  Synthetic sequence hzK2.4E VH M69L, R71A
QVQLVQSGAEVKKPGASVKVSCKASGYTFTSYTMHWVRQAPGQGLEWMGYISPSSGYTEYNQKFKDRVTLTADTSTSTVYMELSSLRSEDTAVYYCARKFDYWGQGTTVTVSS

SEQ ID NO: 94 - protein, 121 aa, Artificial Sequence
  Synthetic sequence hzL4L VH
QVQLQESGPGLVKPSQTLSLTCTVSGGSISSDYAWNWIRQHPGKGLEWIGYISYSGSTRYNPSLKSRVTISVDTSKNQFSLKLSSVTAADTAVYYCARRYRYDEGYGMDYWGQGTTVTVSS

SEQ ID NO: 95 - protein, 121 aa, Artificial Sequence
  Synthetic sequence hzL4.1L VH S30T, V67I, T68S, S70T, V71R
QVQLQESGPGLVKPSQTLSLTCTVSGGSITSDYAWNWIRQHPGKGLEWIGYISYSGSTRYNPSLKSRISITRDTSKNQFSLKLSSVTAADTAVYYCARRYRYDEGYGMDYWGQGTTVTVSS

SEQ ID NO: 96 - protein, 121 aa, Artificial Sequence
  Synthetic sequence hzL4.2L VH G27Y, S30T, V67I, T68S, S70T, V71R
QVQLQESGPGLVKPSQTLSLTCTVSGYSITSDYAWNWIRQHPGKGLEWIGYISYSGSTRYNPSLKSRISITRDTSKNQFSLKLSSVTAADTAVYYCARRYRYDEGYGMDYWGQGTTVTVSS

SEQ ID NO: 97 - protein, 121 aa, Artificial Sequence
  Synthetic sequence hzL4.3L VH G27Y, S30T
QVQLQESGPGLVKPSQTLSLTCTVSGYSITSDYAWNWIRQHPGKGLEWIGYISYSGSTRYNPSLKSRVTISVDTSKNQFSLKLSSVTAADTAVYYCARRYRYDEGYGMDYWGQGTTVTVSS

SEQ ID NO: 98 - protein, 121 aa, Artificial Sequence
  Synthetic sequence hzL4.4L VH G27Y, V67I, T68S, S70T, V71R
QVQLQESGPGLVKPSQTLSLTCTVSGYSISSDYAWNWIRQHPGKGLEWIGYISYSGSTRYNPSLKSRISITRDTSKNQFSLKLSSVTAADTAVYYCARRYRYDEGYGMDYWGQGTTVTVSS

SEQ ID NO: 99 - protein, 121 aa, Artificial Sequence
  Synthetic sequence hzL4.5L VH G27Y, S30T, S70T, V71R
QVQLQESGPGLVKPSQTLSLTCTVSGYSITSDYAWNWIRQHPGKGLEWIGYISYSGSTRYNPSLKSRVTITRDTSKNQFSLKLSSVTAADTAVYYCARRYRYDEGYGMDYWGQGTTVTVSS

SEQ ID NO: 100 - protein, 121 aa, Artificial Sequence
  Synthetic sequence hzL4.6L VH G27Y, S70T, V71R
QVQLQESGPGLVKPSQTLSLTCTVSGYSISSDYAWNWIRQHPGKGLEWIGYISYSGSTRYNPSLKSRVTITRDTSKNQFSLKLSSVTAADTAVYYCARRYRYDEGYGMDYWGQGTTVTVSS

SEQ ID NO: 101 - protein, 121 aa, Artificial Sequence
  Synthetic sequence hzL4.7L VH G27Y, S30T, V67I, T68S
QVQLQESGPGLVKPSQTLSLTCTVSGYSITSDYAWNWIRQHPGKGLEWIGYISYSGSTRYNPSLKSRISISVDTSKNQFSLKLSSVTAADTAVYYCARRYRYDEGYGMDYWGQGTTVTVSS

SEQ ID NO: 102 - protein, 121 aa, Artificial Sequence
  Synthetic sequence hzL4.8L VH G27Y, V67I, T68S
QVQLQESGPGLVKPSQTLSLTCTVSGYSISSDYAWNWIRQHPGKGLEWIGYISYSGSTRYNPSLKSRISISVDTSKNQFSLKLSSVTAADTAVYYCARRYRYDEGYGMDYWGQGTTVTVSS

SEQ ID NO: 103 - protein, 123 aa, Artificial Sequence
  Synthetic sequence hzH8K VH
QVQLQESGPGLVKPSQTLSLTCTVSGGSISSDYAWNWIRQHPGKGLEWIGYRSYRGSTSYNPSLKSRVTISVDTSKNQFSLKLSSVTAADTAVYYCARRGYYGSSSHWYFDVWGQGTTVTVSS

SEQ ID NO: 104 - protein, 123 aa, Artificial Sequence
  Synthetic sequence hzH8.1K VH S30T
QVQLQESGPGLVKPSQTLSLTCTVSGGSITSDYAWNWIRQHPGKGLEWIGYRSYRGSTSYNPSLKSRVTISVDTSKNQFSLKLSSVTAADTAVYYCARRGYYGSSSHWYFDVWGQGTTVTVSS

SEQ ID NO: 105 - protein, 123 aa, Artificial Sequence
  Synthetic sequence hzH8.2K VH G27F, S30T
QVQLQESGPGLVKPSQTLSLTCTVSGFSITSDYAWNWIRQHPGKGLEWIGYRSYRGSTSYNPSLKSRVTISVDTSKNQFSLKLSSVTAADTAVYYCARRGYYGSSSHWYFDVWGQGTTVTVSS

SEQ ID NO: 106 - protein, 123 aa, Artificial Sequence
  Synthetic sequence hzH8.3K VH V67I, T68S, S70T, V71R
QVQLQESGPGLVKPSQTLSLTCTVSGGSISSDYAWNWIRQHPGKGLEWIGYRSYRGSTSYNPSLKSRISITRDTSKNQFSLKLSSVTAADTAVYYCARRGYYGSSSHWYFDVWGQGTTVTVSS

SEQ ID NO: 107 - protein, 123 aa, Artificial Sequence
  Synthetic sequence hzH8.4K VH S30T, V67I, T68S, S70T, V71R
QVQLQESGPGLVKPSQTLSLTCTVSGGSITSDYAWNWIRQHPGKGLEWIGYRSYRGSTSYNPSLKSRISITRDTSKNQFSLKLSSVTAADTAVYYCARRGYYGSSSHWYFDVWGQGTTVTVSS

SEQ ID NO: 108 - protein, 123 aa, Artificial Sequence
  Synthetic sequence hzH8.5K VH G27F, S30T, V67I, T68S, S70T, V71R
QVQLQESGPGLVKPSQTLSLTCTVSGFSITSDYAWNWIRQHPGKGLEWIGYRSYRGSTSYNPSLKSRISITRDTSKNQFSLKLSSVTAADTAVYYCARRGYYGSSSHWYFDVWGQGTTVTVSS

SEQ ID NO: 109 - protein, 123 aa, Artificial Sequence
  Synthetic sequence hzH8.6K VH S30T, V67I, T68S
QVQLQESGPGLVKPSQTLSLTCTVSGGSITSDYAWNWIRQHPGKGLEWIGYRSYRGSTSYNPSLKSRISISVDTSKNQFSLKLSSVTAADTAVYYCARRGYYGSSSHWYFDVWGQGTTVTVSS

SEQ ID NO: 110 - protein, 123 aa, Artificial Sequence
  Synthetic sequence hzH8.7K VH S30T, S70T, V71R
QVQLQESGPGLVKPSQTLSLTCTVSGGSITSDYAWNWIRQHPGKGLEWIGYRSYRGSTSYNPSLKSRVTITRDTSKNQFSLKLSSVTAADTAVYYCARRGYYGSSSHWYFDVWGQGTTVTVSS

SEQ ID NO: 111 - protein, 123 aa, Artificial Sequence
  Synthetic sequence hzH8.8K VH V67I, T68S
QVQLQESGPGLVKPSQTLSLTCTVSGGSISSDYAWNWIRQHPGKGLEWIGYRSYRGSTSYNPSLKSRISISVDTSKNQFSLKLSSVTAADTAVYYCARRGYYGSSSHWYFDVWGQGTTVTVSS

SEQ ID NO: 112 - protein, 123 aa, Artificial Sequence
  Synthetic sequence hzH8.9K VH S70T, V71R
QVQLQESGPGLVKPSQTLSLTCTVSGGSISSDYAWNWIRQHPGKGLEWIGYRSYRGSTSYNPSLKSRVTITRDTSKNQFSLKLSSVTAADTAVYYCARRGYYGSSSHWYFDVWGQGTTVTVSS

SEQ ID NO: 113 - protein, 115 aa, Artificial Sequence
  Synthetic sequence hzJ4F VH
QVQLVQSGAEVKKPGASVKVSCKASGYTFTSYWINWVRQAPGQGLEWMGRIVPGSGSTYYNEMFKDRVTMTRDTSTSTVYMELSSLRSEDTAVYYCARKPNFDVWGQGTTVTVSS

SEQ ID NO: 114 - protein, 115 aa, Artificial Sequence
  Synthetic sequence hzJ4.1F VH V37I
QVQLVQSGAEVKKPGASVKVSCKASGYTFTSYWINWIRQAPGQGLEWMGRIVPGSGSTYYNEMFKDRVTMTRDTSTSTVYMELSSLRSEDTAVYYCARKPNFDVWGQGTTVTVSS

SEQ ID NO: 115 - protein, 115 aa, Artificial Sequence
  Synthetic sequence hzJ4.2F VH V37I, R38K, M48I
QVQLVQSGAEVKKPGASVKVSCKASGYTFTSYWINWIKQAPGQGLEWIGRIVPGSGSTYYNEMFKDRVTMTRDTSTSTVYMELSSLRSEDTAVYYCARKPNFDVWGQGTTVTVSS

SEQ ID NO: 116 - protein, 115 aa, Artificial Sequence
  Synthetic sequence hzJ4.3F VH V37I, R66K, V67A
QVQLVQSGAEVKKPGASVKVSCKASGYTFTSYWINWIRQAPGQGLEWMGRIVPGSGSTYYNEMFKDKATMTRDTSTSTVYMELSSLRSEDTAVYYCARKPNFDVWGQGTTVTVSS

SEQ ID NO: 117 - protein, 115 aa, Artificial Sequence
  Synthetic sequence hzJ4.4F VH V37I, R38K, M48I, R66K, V67A
QVQLVQSGAEVKKPGASVKVSCKASGYTFTSYWINWIKQAPGQGLEWIGRIVPGSGSTYYNEMFKDKATMTRDTSTSTVYMELSSLRSEDTAVYYCARKPNFDVWGQGTTVTVSS

SEQ ID NO: 118 - protein, 115 aa, Artificial Sequence
  Synthetic sequence hzJ4.5F VH R66K, V67A
QVQLVQSGAEVKKPGASVKVSCKASGYTFTSYWINWVRQAPGQGLEWMGRIVPGSGSTYYNEMFKDKATMTRDTSTSTVYMELSSLRSEDTAVYYCARKPNFDVWGQGTTVTVSS

SEQ ID NO: 119 - protein, 115 aa, Artificial Sequence
  Synthetic sequence hzJ4.6F VH R66K, V67A, V78A
QVQLVQSGAEVKKPGASVKVSCKASGYTFTSYWINWVRQAPGQGLEWMGRIVPGSGSTYYNEMFKDKATMTRDTSTSTAYMELSSLRSEDTAVYYCARKPNFDVWGQGTTVTVSS

SEQ ID NO: 120 - protein, 115 aa, Artificial Sequence
  Synthetic sequence hzJ4.7F VH R66K, V67A, V78A, M80I, E81Q
QVQLVQSGAEVKKPGASVKVSCKASGYTFTSYWINWVRQAPGQGLEWMGRIVPGSGSTYYNEMFKDKATMTRDTSTSTAYIQLSSLRSEDTAVYYCARKPNFDVWGQGTTVTVSS

SEQ ID NO: 121 - protein, 115 aa, Artificial Sequence
  Synthetic sequence hzJ4.8F VH R66K, V67A, M69L, R71V
QVQLVQSGAEVKKPGASVKVSCKASGYTFTSYWINWVRQAPGQGLEWMGRIVPGSGSTYYNEMFKDKATLTVDTSTSTVYMELSSLRSEDTAVYYCARKPNFDVWGQGTTVTVSS

SEQ ID NO: 122 - protein, 115 aa, Artificial Sequence
  Synthetic sequence hzJ4.9F VH M69L, R71V
QVQLVQSGAEVKKPGASVKVSCKASGYTFTSYWINWVRQAPGQGLEWMGRIVPGSGSTYYNEMFKDRVTLTVDTSTSTVYMELSSLRSEDTAVYYCARKPNFDVWGQGTTVTVSS

SEQ ID NO: 123 - protein, 115 aa, Artificial Sequence
  Synthetic sequence hzJ4.10F VH R71V
QVQLVQSGAEVKKPGASVKVSCKASGYTFTSYWINWVRQAPGQGLEWMGRIVPGSGSTYYNEMFKDRVTMTVDTSTSTVYMELSSLRSEDTAVYYCARKPNFDVWGQGTTVTVSS

SEQ ID NO: 124 - protein, 115 aa, Artificial Sequence
  Synthetic sequence hzJ4.11F VH V78A
QVQLVQSGAEVKKPGASVKVSCKASGYTFTSYWINWVRQAPGQGLEWMGRIVPGSGSTYYNEMFKDRVTMTRDTSTSTAYMELSSLRSEDTAVYYCARKPNFDVWGQGTTVTVSS

SEQ ID NO: 125 - protein, 115 aa, Artificial Sequence
  Synthetic sequence hzJ4.12F VH V78A, M80I, E81Q
QVQLVQSGAEVKKPGASVKVSCKASGYTFTSYWINWVRQAPGQGLEWMGRIVPGSGSTYYNEMFKDRVTMTRDTSTSTAYIQLSSLRSEDTAVYYCARKPNFDVWGQGTTVTVSS

SEQ ID NO: 126 - protein, 107 aa, Artificial Sequence
  Synthetic sequence G2D VL
DIKMTQSPSSMYASLGERVTITCKASQVINSYLSWFQQKPGKSPETLIYRANTLVDGVPSRFSGSGSGQDYSLTISSLEYEDLGIYYCLQYAEFPYTFGGGTKLEIK

SEQ ID NO: 127 - protein, 107 aa, Artificial Sequence
  Synthetic sequence K2E VL
DIKMTQSPSSMYASLGERVTITCKASQDINSYLSWFQQKPGKSPKTLIYRANRLVDGVPSRFSGSGSGQDFSLTISSLEYEDMGIYYCLQYDEFPFTFGSGTKLEIK

SEQ ID NO: 128 - protein, 108 aa, Artificial Sequence
  Synthetic sequence L4L VL
DIQMTQSPASLSVFVGEIVTITCRASENIYSNLAWYQQKQGKSPQLLVYAATNLADGVPSRFTGSGSGTQFSLKINSLQSEDFGNYYCQHFWGTPPWTFGGGTKLEIK

SEQ ID NO: 129 - protein, 107 aa, Artificial Sequence
  Synthetic sequence H8K VL
DIQMTQSPASLSASVGETVTITCRASENIYSYLAWYQQKQGKSPQLLVYNAKTLAEGVPSRFSGSGSGTQFSLKINSLQPEDFGSYYCQHHYGTPLTFGSGTKLELK

SEQ ID NO: 130 - protein, 107 aa, Artificial Sequence
  Synthetic sequence J4F VL
DIVMTQSQKFMSTSVGDRVSVTCKASQNVGTNVAWYQQKPGQSPKAVIYSASYRFSGVPDRLTGSGSGTDFTLTITNVQSEDLAEYFCQQYNSYPLTFGAGTKLELK

SEQ ID NO: 131 - protein, 107 aa, Artificial Sequence
  Synthetic sequence hzG2D VLa
DIQMTQSPSSLSASVGDRVTITCKASQVINSYLSWFQQKPGKAPKSLIYRANTLVDGVPSRFSGSGSGTDFTLTISSLQPEDFATYYCLQYAEFPYTFGGGTKVEIK

SEQ ID NO: 132 - protein, 107 aa, Artificial Sequence
  Synthetic sequence hzG2.1D VLa D56S
DIQMTQSPSSLSASVGDRVTITCKASQVINSYLSWFQQKPGKAPKSLIYRANTLVSGVPSRFSGSGSGTDFTLTISSLQPEDFATYYCLQYAEFPYTFGGGTKVEIK

SEQ ID NO: 133 - protein, 107 aa, Artificial Sequence
  Synthetic sequence hzG2.2D VLa D56E
DIQMTQSPSSLSASVGDRVTITCKASQVINSYLSWFQQKPGKAPKSLIYRANTLVEGVPSRFSGSGSGTDFTLTISSLQPEDFATYYCLQYAEFPYTFGGGTKVEIK

SEQ ID NO: 134 - protein, 107 aa, Artificial Sequence
  Synthetic sequence hzK2E VL
DIQMTQSPSSLSASVGDRVTITCKASQDINSYLSWFQQKPGKAPKSLIYRANRLVDGVPSRFSGSGSGTDFTLTISSLQPEDFATYYCLQYDEFPFTFGQGTKLEIK

SEQ ID NO: 135 - protein, 107 aa, Artificial Sequence
  Synthetic sequence hzK2.1E VL S46T
DIQMTQSPSSLSASVGDRVTITCKASQDINSYLSWFQQKPGKAPKTLIYRANRLVDGVPSRFSGSGSGTDFTLTISSLQPEDFATYYCLQYDEFPFTFGQGTKLEIK

SEQ ID NO: 136 - protein, 107 aa, Artificial Sequence
  Synthetic sequence hzK2.2E VL D56E
DIQMTQSPSSLSASVGDRVTITCKASQDINSYLSWFQQKPGKAPKSLIYRANRLVEGVPSRFSGSGSGTDFTLTISSLQPEDFATYYCLQYDEFPFTFGQGTKLEIK

SEQ ID NO: 137 - protein, 107 aa, Artificial Sequence
  Synthetic sequence hzK2.3E VL S46T D56E
DIQMTQSPSSLSASVGDRVTITCKASQDINSYLSWFQQKPGKAPKTLIYRANRLVEGVPSRFSGSGSGTDFTLTISSLQPEDFATYYCLQYDEFPFTFGQGTKLEIK

SEQ ID NO: 138 - protein, 108 aa, Artificial Sequence
  Synthetic sequence hzL4L VL
DIQMTQSPSSLSASVGDRVTITCRASENIYSNLAWYQQKPGKAPKLLIYAATNLADGVPSRFSGSGSGTDFTLTISSLQPEDFATYYCQHFWGTPPWTFGGGTKVEIK

SEQ ID NO: 139 - protein, 108 aa, Artificial Sequence
  Synthetic sequence hzL4.1L VL D56S
DIQMTQSPSSLSASVGDRVTITCRASENIYSNLAWYQQKPGKAPKLLIYAATNLASGVPSRFSGSGSGTDFTLTISSLQPEDFATYYCQHFWGTPPWTFGGGTKVEIK

SEQ ID NO: 140 - protein, 108 aa, Artificial Sequence
  Synthetic sequence hzL4.2L VL D56E
DIQMTQSPSSLSASVGDRVTITCRASENIYSNLAWYQQKPGKAPKLLIYAATNLAEGVPSRFSGSGSGTDFTLTISSLQPEDFATYYCQHFWGTPPWTFGGGTKVEIK

SEQ ID NO: 141 - protein, 108 aa, Artificial Sequence
  Synthetic sequence hzL4.3L VL G57A
DIQMTQSPSSLSASVGDRVTITCRASENIYSNLAWYQQKPGKAPKLLIYAATNLADAVPSRFSGSGSGTDFTLTISSLQPEDFATYYCQHFWGTPPWTFGGGTKVEIK

SEQ ID NO: 142 - protein, 107 aa, Artificial Sequence
  Synthetic sequence hzH8K VL
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPLTFGGGTKVEIK

SEQ ID NO: 143 - protein, 107 aa, Artificial Sequence
  Synthetic sequence hzJ4F VL
DIQMTQSPSSLSASVGDRVTITCKASQNVGTNVAWYQQKPGKAPKLLIYSASYRFSGVPSRFSGSGSGTDFTLTISSLQPEDFATYYCQQYNSYPLTFGGGTKVEIK

SEQ ID NO: 144 - protein, 107 aa, Artificial Sequence
  Synthetic sequence hzJ4.1F VL Q3V
DIVMTQSPSSLSASVGDRVTITCKASQNVGTNVAWYQQKPGKAPKLLIYSASYRFSGVPSRFSGSGSGTDFTLTISSLQPEDFATYYCQQYNSYPLTFGGGTKVEIK

SEQ ID NO: 145 - protein, 107 aa, Artificial Sequence
  Synthetic sequence hzJ4.2F VL Q3V L46A
DIVMTQSPSSLSASVGDRVTITCKASQNVGTNVAWYQQKPGKAPKALIYSASYRFSGVPSRFSGSGSGTDFTLTISSLQPEDFATYYCQQYNSYPLTFGGGTKVEIK

SEQ ID NO: 146 - protein, 107 aa, Artificial Sequence
  Synthetic sequence hzJ4.3F VL L46A
DIQMTQSPSSLSASVGDRVTITCKASQNVGTNVAWYQQKPGKAPKALIYSASYRFSGVPSRFSGSGSGTDFTLTISSLQPEDFATYYCQQYNSYPLTFGGGTKVEIK

SEQ ID NO: 147 - protein, 107 aa, Artificial Sequence
  Synthetic sequence hzJ4.4F VL L46A, L47V
DIQMTQSPSSLSASVGDRVTITCKASQNVGTNVAWYQQKPGKAPKAVIYSASYRFSGVPSRFSGSGSGTDFTLTISSLQPEDFATYYCQQYNSYPLTFGGGTKVEIK

SEQ ID NO: 148 - protein, 107 aa, Artificial Sequence
  Synthetic sequence hzJ4.5F VL L46A, L47V, F62L
DIQMTQSPSSLSASVGDRVTITCKASQNVGTNVAWYQQKPGKAPKAVIYSASYRFSGVPSRLSGSGSGTDFTLTISSLQPEDFATYYCQQYNSYPLTFGGGTKVEIK

SEQ ID NO: 149 - protein, 107 aa, Artificial Sequence
  Synthetic sequence hzJ4.6F VL L46A, L47V, S60D, F62L, S63T
DIQMTQSPSSLSASVGDRVTITCKASQNVGTNVAWYQQKPGKAPKAVIYSASYRFSGVPDRLTGSGSGTDFTLTISSLQPEDFATYYCQQYNSYPLTFGGGTKVEIK

SEQ ID NO: 150 - protein, 444 aa, Artificial Sequence
  Synthetic sequence chG2D Heavy Chain
EVQLQQSGPELVKPGASVKISCKASGYSFTGYYMHWVKQSHVKSLEWIGRINPNNGATNYNQNFKDKASLTVDKSSSTAYMEFHSLTSEDSAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 443 aa, Artificial Sequence
  Synthetic sequence chK2E Heavy Chain
QVQLQQSAAELARPGASVKMSCKASGYTFTSYTMHWVKQRPGQGLEWIGYISPSSGYTEYNQKFKDKTTLTADKSSSTAYMQLSSLTSEDSAVYYCARKFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 451 aa, Artificial Sequence
  Synthetic sequence chL4L Heavy Chain
DVQLQESGPGLVKPSQSLSLTCTVTGYSITSDYAWNWIRQFPGNKLEWMGYISYSGSTRYNPSLKSRISITRDTSKNQFFLQLNSVTTEDTATYYCARRYRYDEGYGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 453 aa, Artificial Sequence
  Synthetic sequence chH8K Heavy Chain
DVQLQESGPGLVKPSQSLSLTCTVTGFSITSDYAWNWIRQFPGNQLEWMGYRSYRGSTSYNPSLKSRISITRDTSKNQFFLQLNSVTTEDTATYYCARRGYYGSSSHWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 445 aa, Artificial Sequence
  Synthetic sequence chJ4F Heavy Chain
QVQLQQSGDDLVKPGASVKLSCKASGYTFTSYWINWIKQRPGQGLEWIGRIVPGSGSTYYNEMFKDKATLTVDTSSSTAYIQLSSLSSEDSAVYFCARKPNFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 444 aa, Artificial Sequence
  Synthetic sequence hzG2D Heavy Chain V109L
QVQLVQSGAEVKKPGASVKVSCKASGYTFTGYYMHWVRQAPGQRLEWMGRINPNNGATNYNQNFKDRVTITRDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 444 aa, Artificial Sequence
  Synthetic sequence hzG2.1D Heavy Chain T28S, V109L
QVQLVQSGAEVKKPGASVKVSCKASGYSFTGYYMHWVRQAPGQRLEWMGRINPNNGATNYNQNFKDRVTITRDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 444 aa, Artificial Sequence
  Synthetic sequence hzG2.2D Heavy Chain T28S, R66K, V67A, V109L
QVQLVQSGAEVKKPGASVKVSCKASGYSFTGYYMHWVRQAPGQRLEWMGRINPNNGATNYNQNFKDKATITRDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 444 aa, Artificial Sequence
  Synthetic sequence hzG2.3D Heavy Chain T28S, R66K, V67A, A93T, R94S, V109L
QVQLVQSGAEVKKPGASVKVSCKASGYSFTGYYMHWVRQAPGQRLEWMGRINPNNGATNYNQNFKDKATITRDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 444 aa, Artificial Sequence
  Synthetic sequence hzG2.4D Heavy Chain T28S, R66K, V67A, T68S, I69L, R71V, V109L
QVQLVQSGAEVKKPGASVKVSCKASGYSFTGYYMHWVRQAPGQRLEWMGRINPNNGATNYNQNFKDKASLTVDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 444 aa, Artificial Sequence
  Synthetic sequence hzG2.5D Heavy Chain T28S, R66K, V67A, T68S, I69L, R71V, A93T, R94S, V109L
QVQLVQSGAEVKKPGASVKVSCKASGYSFTGYYMHWVRQAPGQRLEWMGRINPNNGATNYNQNFKDKASLTVDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 444 aa, Artificial Sequence
  Synthetic sequence hzG2.6D Heavy Chain T28S, T68S, I69L, R71V, V109L
QVQLVQSGAEVKKPGASVKVSCKASGYSFTGYYMHWVRQAPGQRLEWMGRINPNNGATNYNQNFKDRVSLTVDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 444 aa, Artificial Sequence
  Synthetic sequence hzG2.7D Heavy Chain T28S, T68S, I69L, R71V, A93T, R94S, V109L
QVQLVQSGAEVKKPGASVKVSCKASGYSFTGYYMHWVRQAPGQRLEWMGRINPNNGATNYNQNFKDRVSLTVDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 444 aa, Artificial Sequence
  Synthetic sequence hzG2.8D Heavy Chain T28S, A93T, R94S, V109L
QVQLVQSGAEVKKPGASVKVSCKASGYSFTGYYMHWVRQAPGQRLEWMGRINPNNGATNYNQNFKDRVTITRDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 444 aa, Artificial Sequence
  Synthetic sequence hzG2.9D Heavy Chain T28S, N54Q
QVQLVQSGAEVKKPGASVKVSCKASGYSFTGYYMHWVRQAPGQRLEWMGRINPNQGATNYNQNFKDRVTITRDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 444 aa, Artificial Sequence
  Synthetic sequence hzG2.10D Heavy Chain T28S, N54Q, R66K, V67A
QVQLVQSGAEVKKPGASVKVSCKASGYSFTGYYMHWVRQAPGQRLEWMGRINPNQGATNYNQNFKDKATITRDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 444 aa, Artificial Sequence
  Synthetic sequence hzG2.11D Heavy Chain T28S, N54Q, R66K, V67A, A93T, R94S
QVQLVQSGAEVKKPGASVKVSCKASGYSFTGYYMHWVRQAPGQRLEWMGRINPNQGATNYNQNFKDKATITRDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 444 aa, Artificial Sequence
  Synthetic sequence hzG2.12D Heavy Chain T28S, N54Q, R66K, V67A, T68S, I69L, R71V
QVQLVQSGAEVKKPGASVKVSCKASGYSFTGYYMHWVRQAPGQRLEWMGRINPNQGATNYNQNFKDKASLTVDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 444 aa, Artificial Sequence
  Synthetic sequence hzG2.13D Heavy Chain T28S, N54Q, R66K, V67A, T68S, I69L, R71V, A93T, R94S
QVQLVQSGAEVKKPGASVKVSCKASGYSFTGYYMHWVRQAPGQRLEWMGRINPNQGATNYNQNFKDKASLTVDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 444 aa, Artificial Sequence
  Synthetic sequence hzG2.14D Heavy Chain T28S, N54Q, T68S, I69L, R71V
QVQLVQSGAEVKKPGASVKVSCKASGYSFTGYYMHWVRQAPGQRLEWMGRINPNQGATNYNQNFKDRVSLTVDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 444 aa, Artificial Sequence
  Synthetic sequence hzG2.15D Heavy Chain T28S, N54Q, T68S, I69L, R71V, A93T, R94S
QVQLVQSGAEVKKPGASVKVSCKASGYSFTGYYMHWVRQAPGQRLEWMGRINPNQGATNYNQNFKDRVSLTVDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 444 aa, Artificial Sequence
  Synthetic sequence hzG2.16D Heavy Chain T28S, N54Q, A93T, R94S
QVQLVQSGAEVKKPGASVKVSCKASGYSFTGYYMHWVRQAPGQRLEWMGRINPNQGATNYNQNFKDRVTITRDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 444 aa, Artificial Sequence
  Synthetic sequence hzG2.17D Heavy Chain T28S, G55A
QVQLVQSGAEVKKPGASVKVSCKASGYSFTGYYMHWVRQAPGQRLEWMGRINPNNAATNYNQNFKDRVTITRDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 444 aa, Artificial Sequence
  Synthetic sequence hzG2.18D Heavy Chain T28S, G55A, R66K, V67A
QVQLVQSGAEVKKPGASVKVSCKASGYSFTGYYMHWVRQAPGQRLEWMGRINPNNAATNYNQNFKDKATITRDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 444 aa, Artificial Sequence
  Synthetic sequence hzG2.19D Heavy Chain T28S, G55A, R66K, V67A, A93T, R94S
QVQLVQSGAEVKKPGASVKVSCKASGYSFTGYYMHWVRQAPGQRLEWMGRINPNNAATNYNQNFKDKATITRDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 444 aa, Artificial Sequence
  Synthetic sequence hzG2.20D Heavy Chain T28S, G55A, R66K, V67A, T68S, I69L, R71V
QVQLVQSGAEVKKPGASVKVSCKASGYSFTGYYMHWVRQAPGQRLEWMGRINPNNAATNYNQNFKDKASLTVDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 444 aa, Artificial Sequence
  Synthetic sequence hzG2.21D Heavy Chain T28S, G55A, R66K, V67A, T68S, I69L, R71V, A93T, R94S
QVQLVQSGAEVKKPGASVKVSCKASGYSFTGYYMHWVRQAPGQRLEWMGRINPNNAATNYNQNFKDKASLTVDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 444 aa, Artificial Sequence
  Synthetic sequence hzG2.22D Heavy Chain T28S, G55A, T68S, I69L, R71V
QVQLVQSGAEVKKPGASVKVSCKASGYSFTGYYMHWVRQAPGQRLEWMGRINPNNAATNYNQNFKDRVSLTVDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 444 aa, Artificial Sequence
  Synthetic sequence hzG2.23D Heavy Chain T28S, G55A, T68S, I69L, R71V, A93T, R94S
QVQLVQSGAEVKKPGASVKVSCKASGYSFTGYYMHWVRQAPGQRLEWMGRINPNNAATNYNQNFKDRVSLTVDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 444 aa, Artificial Sequence
  Synthetic sequence hzG2.24D Heavy Chain T28S, G55A, A93T, R94S
QVQLVQSGAEVKKPGASVKVSCKASGYSFTGYYMHWVRQAPGQRLEWMGRINPNNAATNYNQNFKDRVTITRDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 444 aa, Artificial Sequence
  Synthetic sequence hzG2.25D Heavy Chain N54Q, R66K, V67A
QVQLVQSGAEVKKPGASVKVSCKASGYTFTGYYMHWVRQAPGQRLEWMGRINPNQGATNYNQNFKDKATITRDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 444 aa, Artificial Sequence
  Synthetic sequence hzG2.26D Heavy Chain N54Q, R66K, V67A, A93T, R94S
QVQLVQSGAEVKKPGASVKVSCKASGYTFTGYYMHWVRQAPGQRLEWMGRINPNQGATNYNQNFKDKATITRDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 444 aa, Artificial Sequence
  Synthetic sequence hzG2.27D Heavy Chain N54Q, R66K, V67A, T68S, I69L, R71V
QVQLVQSGAEVKKPGASVKVSCKASGYTFTGYYMHWVRQAPGQRLEWMGRINPNQGATNYNQNFKDKASLTVDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 444 aa, Artificial Sequence
  Synthetic sequence hzG2.28D Heavy Chain N54Q, R66K, V67A, T68S, I69L, R71V, A93T, R94S
QVQLVQSGAEVKKPGASVKVSCKASGYTFTGYYMHWVRQAPGQRLEWMGRINPNQGATNYNQNFKDKASLTVDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 444 aa, Artificial Sequence
  Synthetic sequence hzG2.29D Heavy Chain N54Q, T68S, I69L, R71V
QVQLVQSGAEVKKPGASVKVSCKASGYTFTGYYMHWVRQAPGQRLEWMGRINPNQGATNYNQNFKDRVSLTVDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 444 aa, Artificial Sequence
  Synthetic sequence hzG2.30D Heavy Chain N54Q, T68S, I69L, R71V, A93T, R94S
QVQLVQSGAEVKKPGASVKVSCKASGYTFTGYYMHWVRQAPGQRLEWMGRINPNQGATNYNQNFKDRVSLTVDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 444 aa, Artificial Sequence
  Synthetic sequence hzG2.31D Heavy Chain N54Q, A93T, R94S
QVQLVQSGAEVKKPGASVKVSCKASGYTFTGYYMHWVRQAPGQRLEWMGRINPNQGATNYNQNFKDRVTITRDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 444 aa, Artificial Sequence
  Synthetic sequence hzG2.32D Heavy Chain G55A, R66K, V67A
QVQLVQSGAEVKKPGASVKVSCKASGYTFTGYYMHWVRQAPGQRLEWMGRINPNNAATNYNQNFKDKATITRDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 444 aa, Artificial Sequence
  Synthetic sequence hzG2.33D Heavy Chain G55A, R66K, V67A, A93T, R94S
QVQLVQSGAEVKKPGASVKVSCKASGYTFTGYYMHWVRQAPGQRLEWMGRINPNNAATNYNQNFKDKATITRDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 444 aa, Artificial Sequence
  Synthetic sequence hzG2.34D Heavy Chain G55A, R66K, V67A, T68S, I69L, R71V
QVQLVQSGAEVKKPGASVKVSCKASGYTFTGYYMHWVRQAPGQRLEWMGRINPNNAATNYNQNFKDKASLTVDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 444 aa, Artificial Sequence
  Synthetic sequence hzG2.35D Heavy Chain G55A, R66K, V67A, T68S, I69L, R71V, A93T, R94S
QVQLVQSGAEVKKPGASVKVSCKASGYTFTGYYMHWVRQAPGQRLEWMGRINPNNAATNYNQNFKDKASLTVDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 444 aa, Artificial Sequence
  Synthetic sequence hzG2.36D Heavy Chain G55A, T68S, I69L, R71V
QVQLVQSGAEVKKPGASVKVSCKASGYTFTGYYMHWVRQAPGQRLEWMGRINPNNAATNYNQNFKDRVSLTVDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 444 aa, Artificial Sequence
  Synthetic sequence hzG2.37D Heavy Chain G55A, T68S, I69L, R71V, A93T, R94S
QVQLVQSGAEVKKPGASVKVSCKASGYTFTGYYMHWVRQAPGQRLEWMGRINPNNAATNYNQNFKDRVSLTVDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 444 aa, Artificial Sequence
  Synthetic sequence hzG2.38D Heavy Chain G55A, A93T, R94S
QVQLVQSGAEVKKPGASVKVSCKASGYTFTGYYMHWVRQAPGQRLEWMGRINPNNAATNYNQNFKDRVTITRDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 444 aa, Artificial Sequence
  Synthetic sequence hzG2.39D Heavy Chain R66K, V67A, V109L
QVQLVQSGAEVKKPGASVKVSCKASGYTFTGYYMHWVRQAPGQRLEWMGRINPNNGATNYNQNFKDKATITRDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 444 aa, Artificial Sequence
  Synthetic sequence hzG2.40D Heavy Chain R66K, V67A, A93T, R94S, V109L
QVQLVQSGAEVKKPGASVKVSCKASGYTFTGYYMHWVRQAPGQRLEWMGRINPNNGATNYNQNFKDKATITRDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 444 aa, Artificial Sequence
  Synthetic sequence hzG2.41D Heavy Chain R66K, V67A, T68S, I69L, R71V, V109L
QVQLVQSGAEVKKPGASVKVSCKASGYTFTGYYMHWVRQAPGQRLEWMGRINPNNGATNYNQNFKDKASLTVDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 444 aa, Artificial Sequence
  Synthetic sequence hzG2.42D Heavy Chain R66K, V67A, T68S, I69L, R71V, A93T, R94S, V109L
QVQLVQSGAEVKKPGASVKVSCKASGYTFTGYYMHWVRQAPGQRLEWMGRINPNNGATNYNQNFKDKASLTVDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 444 aa, Artificial Sequence
  Synthetic sequence hzG2.43D Heavy Chain T68S, I69L, R71V, V109L
QVQLVQSGAEVKKPGASVKVSCKASGYTFTGYYMHWVRQAPGQRLEWMGRINPNNGATNYNQNFKDRVSLTVDTSASTAYMELSSLRSEDTAVYYCARVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 444 aa, Artificial Sequence
  Synthetic sequence hzG2.44D Heavy Chain T68S, I69L, R71V, A93T, R94S, V109L
QVQLVQSGAEVKKPGASVKVSCKASGYTFTGYYMHWVRQAPGQRLEWMGRINPNNGATNYNQNFKDRVSLTVDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 200 - protein, 444 aa, Artificial Sequence
  Synthetic sequence hzG2.45D Heavy Chain A93T, R94S, V109L
QVQLVQSGAEVKKPGASVKVSCKASGYTFTGYYMHWVRQAPGQRLEWMGRINPNNGATNYNQNFKDRVTITRDTSASTAYMELSSLRSEDTAVYYCTSVNFYYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 443 aa, Artificial Sequence
  Synthetic sequence hzK2E Heavy Chain
QVQLVQSGAEVKKPGASVKVSCKASGYTFTSYTMHWVRQAPGQGLEWMGYISPSSGYTEYNQKFKDRVTMTRDTSTSTVYMELSSLRSEDTAVYYCARKFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 443 aa, Artificial Sequence
  Synthetic sequence hzK2.1E Heavy Chain R66K, V67T
QVQLVQSGAEVKKPGASVKVSCKASGYTFTSYTMHWVRQAPGQGLEWMGYISPSSGYTEYNQKFKDKTTMTRDTSTSTVYMELSSLRSEDTAVYYCARKFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - protein, 443 aa, Artificial Sequence
  Synthetic sequence hzK2.2E Heavy Chain R66K, V67T, M69L, R71A
QVQLVQSGAEVKKPGASVKVSCKASGYTFTSYTMHWVRQAPGQGLEWMGYISPSSGYTEYNQKFKDKTTLTADTSTSTVYMELSSLRSEDTAVYYCARKFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - protein, 443 aa, Artificial Sequence
  Synthetic sequence hzK2.3E Heavy Chain R66K, V67T, M69L, R71A, T73K
QVQLVQSGAEVKKPGASVKVSCKASGYTFTSYTMHWVRQAPGQGLEWMGYISPSSGYTEYNQKFKDKTTLTADKSTSTVYMELSSLRSEDTAVYYCARKFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205 - protein, 443 aa, Artificial Sequence
  Synthetic sequence hzK2.4E Heavy Chain M69L, R71A
QVQLVQSGAEVKKPGASVKVSCKASGYTFTSYTMHWVRQAPGQGLEWMGYISPSSGYTEYNQKFKDRVTLTADTSTSTVYMELSSLRSEDTAVYYCARKFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 214 aa, Artificial Sequence
  Synthetic sequence chG2D Light Chain
DIKMTQSPSSMYASLGERVTITCKASQVINSYLSWFQQKPGKSPETLIYRANTLVDGVPSRFSGSGSGQDYSLTISSLEYEDLGIYYCLQYAEFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - protein, 214 aa, Artificial Sequence
  Synthetic sequence chK2E Light Chain
DIKMTQSPSSMYASLGERVTITCKASQDINSYLSWFQQKPGKSPKTLIYRANRLVDGVPSRFSGSGSGQDFSLTISSLEYEDMGIYYCLQYDEFPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 208 - protein, 215 aa, Artificial Sequence
  Synthetic sequence chL4L Light Chain
DIQMTQSPASLSVFVGEIVTITCRASENIYSNLAWYQQKQGKSPQLLVYAATNLADGVPSRFTGSGSGTQFSLKINSLQSEDFGNYYCQHFWGTPPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - protein, 214 aa, Artificial Sequence
  Synthetic sequence chH8K Light Chain
DIQMTQSPASLSASVGETVTITCRASENIYSYLAWYQQKQGKSPQLLVYNAKTLAEGVPSRFSGSGSGTQFSLKINSLQPEDFGSYYCQHHYGTPLTFGSGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 210 - protein, 214 aa, Artificial Sequence
  Synthetic sequence chJ4F Light Chain
DIVMTQSQKFMSTSVGDRVSVTCKASQNVGTNVAWYQQKPGQSPKAVIYSASYRFSGVPDRLTGSGSGTDFTLTITNVQSEDLAEYFCQQYNSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - protein, 214 aa, Artificial Sequence
  Synthetic sequence hzG2D Light Chain
DIQMTQSPSSLSASVGDRVTITCKASQVINSYLSWFQQKPGKAPKSLIYRANTLVDGVPSRFSGSGSGTDFTLTISSLQPEDFATYYCLQYAEFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 212 - protein, 214 aa, Artificial Sequence
  Synthetic sequence hzG2.1D Light Chain D56S
DIQMTQSPSSLSASVGDRVTITCKASQVINSYLSWFQQKPGKAPKSLIYRANTLVSGVPSRFSGSGSGTDFTLTISSLQPEDFATYYCLQYAEFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 213 - protein, 214 aa, Artificial Sequence
  Synthetic sequence hzG2.2D Light Chain D56E
DIQMTQSPSSLSASVGDRVTITCKASQVINSYLSWFQQKPGKAPKSLIYRANTLVEGVPSRFSGSGSGTDFTLTISSLQPEDFATYYCLQYAEFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 214 - protein, 214 aa, Artificial Sequence
  Synthetic sequence hzK2E Light Chain
DIQMTQSPSSLSASVGDRVTITCKASQDINSYLSWFQQKPGKAPKSLIYRANRLVDGVPSRFSGSGSGTDFTLTISSLQPEDFATYYCLQYDEFPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215 - protein, 214 aa, Artificial Sequence
  Synthetic sequence hzK2.1E Light Chain S46T
DIQMTQSPSSLSASVGDRVTITCKASQDINSYLSWFQQKPGKAPKTLIYRANRLVDGVPSRFSGSGSGTDFTLTISSLQPEDFATYYCLQYDEFPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 214 aa, Artificial Sequence
  Synthetic sequence hzK2.2E Light Chain D56E
DIQMTQSPSSLSASVGDRVTITCKASQDINSYLSWFQQKPGKAPKSLIYRANRLVEGVPSRFSGSGSGTDFTLTISSLQPEDFATYYCLQYDEFPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - protein, 214 aa, Artificial Sequence
  Synthetic sequence hzK2.3E Light Chain S46T D56E
DIQMTQSPSSLSASVGDRVTITCKASQDINSYLSWFQQKPGKAPKTLIYRANRLVEGVPSRFSGSGSGTDFTLTISSLQPEDFATYYCLQYDEFPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - protein, 5 aa, Artificial Sequence
  Synthetic sequence ASGR1 mAb-C VH CDR1
SYAMS

SEQ ID NO: 219 - protein, 17 aa, Artificial Sequence
  Synthetic sequence ASGR1 mAb-C VH CDR2
AISGSGGSTYYADSVKG

SEQ ID NO: 220 - protein, 11 aa, Artificial Sequence
  Synthetic sequence ASGR1 mAb-C VH CDR3
DFSSRRWYLEY

SEQ ID NO: 221 - protein, 11 aa, Artificial Sequence
  Synthetic sequence ASGR1 mAb-C VL CDR1
QGDSLRSYYAS

SEQ ID NO: 222 - protein, 7 aa, Artificial Sequence
  Synthetic sequence ASGR1 mAb-C VL CDR2
GKNNRPS

SEQ ID NO: 223 - protein, 12 aa, Artificial Sequence
  Synthetic sequence ASGR1 mAb-C VL CDR3
NSLERIGYLSYV

SEQ ID NO: 224 - protein, 120 aa, Artificial Sequence
  Synthetic sequence ASGR1 mAb-C VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDFSSRRWYLEYWGQGTLVTVSS

SEQ ID NO: 225 - protein, 109 aa, Artificial Sequence
  Synthetic sequence ASGR1 mAb-C VL
SSELTQDPAVSVALGQTVRITCQGDSLRSYYASWYQQKPGQAPVLVIYGKNNRPSGIPDRFSGSSSGNTASLTITGAQAEDEADYYCNSLERIGYLSYVFGGGTKLTVL

SEQ ID NO: 226 - protein, 120 aa, Artificial Sequence
  Synthetic sequence mzASGR1 mAb-C VH
EVKLVESGGGLVKPGGSLKLSCAASGFTFSSYAMSWVRQTPEKRLEWVSAISGSGGSTYYADSVKGRFTISRDNAKNTLYLQMSSLRSEDTAMYYCAKDFSSRRWYLEYWGQGTTLTVSS

SEQ ID NO: 227 - protein, 109 aa, Artificial Sequence
  Synthetic sequence mzASGR1 mAb-C VL
QAVLTQEPALSVSLGQTVTLTCQGDSLRSYYASWYQQKPGQAPVLVIYGKNNRPSGVPARFSGSLIGDKAALTITGAQTEDEAIYYCNSLERIGYLSYVFGGGTKVTVL

SEQ ID NO: 228 - protein, 450 aa, Artificial Sequence
  Synthetic sequence mzASGR1 mAb-C Heavy Chain (VH-IgG2a)
EVKLVESGGGLVKPGGSLKLSCAASGFTFSSYAMSWVRQTPEKRLEWVSAISGSGGSTYYADSVKGRFTISRDNAKNTLYLQMSSLRSEDTAMYYCAKDFSSRRWYLEYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 229 - protein, 216 aa, Artificial Sequence
  Synthetic sequence mzASGR1 mAb-C Light Chain (VL-kappa)
QAVLTQEPALSVSLGQTVTLTCQGDSLRSYYASWYQQKPGQAPVLVIYGKNNRPSGVPARFSGSLIGDKAALTITGAQTEDEAIYYCNSLERIGYLSYVFGGGTKVTVLRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 230 - protein, 330 aa, Artificial Sequence
  Synthetic sequence Human IgG1 Heavy Chain Constant Region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 330 aa, Artificial Sequence
  Synthetic sequence Human IgG1null Heavy Chain Constant Region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 232 - protein, 107 aa, Artificial Sequence [sequence omitted]

SEQ ID NO: 233 - protein, 330 aa, Artificial Sequence
  Synthetic sequence Mouse IgG2a Heavy Chain Constant Region
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 234 - protein, 107 aa, Artificial Sequence
  Synthetic sequence Mouse Kappa Light Chain Constant Region
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 235 - protein, 331 aa, Artificial Sequence
  Synthetic sequence Rat IgG2b Heavy Chain Constant Region
AQTTAPSVYPLAPGCGDTTSSTVTLGCLVKGYFPEPVTVTWNSGALSSDVHTFPAVLQSGLYTLTSSVTSSTWPSQTVTCNVAHPASSTKVDKKVERRNGGIGHKCPTCPTCHKCPVPELLGGPSVFIFPPKPKDILLISQNAKVTCVVVDVSEEEPDVQFSWFVNNVEVHTAQTQPREEQYNSTFRVVSALPIQHQDWMSGKEFKCKVNNKALPSPIEKTISKPKGLVRKPQVYVMGPPTEQLTEQTVSLTCLTSGFLPNDIGVEWTSNGHIEKNYKNTEPVMDSDGSFFMYSKLNVERSRWDSRAPFVCSVVHEGLHNHHVEKSISRPP

SEQ ID NO: 236 - protein, 107 aa, Artificial Sequence
  Synthetic sequence Rat kappa Light Chain Constant Region
RADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 237 - protein, 5 aa, Artificial Sequence
  Synthetic sequence recognition motif
  VARIANT (1)...(5): Xaa = Any Amino Acid
LPXTG

SEQ ID NO: 238 - protein, 114 aa, Artificial Sequence
  Synthetic sequence hzG2.46D VH R66K, V67A, T68S, I69L, R71V
QVQLVQSGAEVKKPGASVKVSCKASGYTFTGYYMHWVRQAPGQRLEWMGRINPNNGATNYNQNFKDKASLTVDTSASTAYMELSSLRSEDTAVYYCARVNFYYWGQGTTVTVSS

SEQ ID NO: 239 - protein, 114 aa, Artificial Sequence
  Synthetic sequence hzG2.47D VH R66K, V67A, T68S, I69L, R71V, A93T, R94S
QVQLVQSGAEVKKPGASVKVSCKASGYTFTGYYMHWVRQAPGQRLEWMGRINPNNGATNYNQNFKDKASLTVDTSASTAYMELSSLRSEDTAVYYCTSVNFYYWGQGTTVTVSS

SEQ ID NO: 240 - protein, 114 aa, Artificial Sequence
  Synthetic sequence hzG2.48D VH G55A, R66K, V67A, T68S, I69L, R71V, A93T, R94S
QVQLVQSGAEVKKPGASVKVSCKASGYTFTGYYMHWVRQAPGQRLEWMGRINPNNAATNYNQNFKDKASLTVDTSASTAYMELSSLRSEDTAVYYCTSVNFYYWGQGTTVTVSS

SEQ ID NO: 241 - protein, 114 aa, Artificial Sequence
  Synthetic sequence hzG2.49D VH R38K, R66K, V67A, T68S, I69L, R71V, A93T, R94S
QVQLVQSGAEVKKPGASVKVSCKASGYTFTGYYMHWVKQAPGQRLEWMGRINPNNGATNYNQNFKDKASLTVDTSASTAYMELSSLRSEDTAVYYCTSVNFYYWGQGTTVTVSS

SEQ ID NO: 242 - protein, 114 aa, Artificial Sequence
  Synthetic sequence hzG2.50D VH R66K, V67A, T68S, I69L, R71V, L82F, S82aH, A93T, R94S
QVQLVQSGAEVKKPGASVKVSCKASGYTFTGYYMHWVRQAPGQRLEWMGRINPNNGATNYNQNFKDKASLTVDTSASTAYMEFHSLRSEDTAVYYCTSVNFYYWGQGTTVTVSS

SEQ ID NO: 243 - protein, 114 aa, Artificial Sequence
  Synthetic sequence hzG2.51D VH R66K, V67A, T68S, I69L, R71V, R84T, A93T, R94S
QVQLVQSGAEVKKPGASVKVSCKASGYTFTGYYMHWVRQAPGQRLEWMGRINPNNGATNYNQNFKDKASLTVDTSASTAYMELSSLTSEDTAVYYCTSVNFYYWGQGTTVTVSS

SEQ ID NO: 244 - protein, 107 aa, Artificial Sequence
  Synthetic sequence hzG2.3D VLa K45E, S46T
DIQMTQSPSSLSASVGDRVTITCKASQVINSYLSWFQQKPGKAPETLIYRANTLVDGVPSRFSGSGSGTDFTLTISSLQPEDFATYYCLQYAEFPYTFGGGTKVEIK

SEQ ID NO: 245 - protein, 107 aa, Artificial Sequence
  Synthetic sequence hzG2.4D VLa Q79E, P80Y
DIQMTQSPSSLSASVGDRVTITCKASQVINSYLSWFQQKPGKAPKSLIYRANTLVDGVPSRFSGSGSGTDFTLTISSLEYEDFATYYCLQYAEFPYTFGGGTKVEIK

SEQ ID NO: 246 - protein, 107 aa, Artificial Sequence
  Synthetic sequence hzG2.5D VLa F71Y, T72S
DIQMTQSPSSLSASVGDRVTITCKASQVINSYLSWFQQKPGKAPKSLIYRANTLVDGVPSRFSGSGSGTDYSLTISSLQPEDFATYYCLQYAEFPYTFGGGTKVEIK

SEQ ID NO: 247 - protein, 107 aa, Artificial Sequence
  Synthetic sequence hzG2D VLb
DIQMTQSPSSLSASVGDRVTITCKASQVINSYLSWYQQKPGKAPKLLIYRANTLVDGVPSRFSGSGSGTDFTLTISSLQPEDFATYYCLQYAEFPYTFGGGTKVEIK

SEQ ID NO: 248 - protein, 107 aa, Artificial Sequence
  Synthetic sequence hzG2.6D VLb D56S
DIQMTQSPSSLSASVGDRVTITCKASQVINSYLSWYQQKPGKAPKLLIYRANTLVSGVPSRFSGSGSGTDFTLTISSLQPEDFATYYCLQYAEFPYTFGGGTKVEIK

SEQ ID NO: 249 - protein, 107 aa, Artificial Sequence
  Synthetic sequence hzG2D VLc
DIQMTQSPSSLSASVGDRVTITCKASQVINSYLSWYQQKPGKVPKLLIYRANTLVDGVPSRFSGSGSGTDFTLTISSLQPEDVATYYCLQYAEFPYTFGGGTKVEIK